(19) United States
(12) Patent Application Publication
(10) Pub. No.: US 2024/0294890 A1
(43) Pub. Date: Sep. 5, 2024

(54) SYSTEMS, METHODS, AND COMPOSITIONS FOR RNA-GUIDED RNA-TARGETING CRISPR EFFECTORS

(71) Applicant: MASSACHUSETTS INSTITUTE OF TECHNOLOGY, Cambridge, MA (US)

(72) Inventors: Omar Abudayyeh, Cambridge, MA (US); Jonathan Gootenberg, Cambridge, MA (US)

(21) Appl. No.: 18/649,077

(22) Filed: Apr. 29, 2024

Related U.S. Application Data
(62) Division of application No. 17/365,777, filed on Jul. 1, 2021, now Pat. No. 12,012,621.
(60) Provisional application No. 63/208,606, filed on Jun. 9, 2021.
(60) Provisional application No. 63/073,898, filed on Sep. 2, 2020.

(51) Int. Cl. C12N 9/22; A61K 38/00; A61K 48/00; C12N 9/78; C12N 15/10; C12N 15/11; C12N 15/63; C12Q 1/6825

(52) U.S. Cl. CPC C12N 9/22; A61K 48/005; C12N 9/78; C12N 15/102; C12N 15/11; C12N 15/63; C12Y 305/04004; C12Y 305/04005; A61K 38/00; C07K 2319/85; C12N 2310/20; C12N 2320/10; C12N 2320/33; C12Q 1/6825

(57) ABSTRACT

This disclosure provides systems, methods, and compositions for RNA-guided RNA-targeting CRISPR effectors for the treatment of diseases as well as diagnostics. In some embodiments, nucleotide deaminase functionalized CRISPR systems for RNA editing RNA knockdown, viral resistance, splicing modulation, RNA tracking, translation modulation, and epi-transcriptomic modifications are disclosed.

### CROSS REFERENCE TO RELATED APPLICATIONS

This application is a divisional of U.S. patent application Ser. No. 17/365,777, filed Jul. 1, 2021, and claims priority to U.S. Provisional Application Nos. 63/073,898, filed Sep. 2, 2020, and 63/208,606, filed Jun. 9, 2021, the entire contents of which are hereby incorporated by reference in their entirety.

### STATEMENT REGARDING FEDERALLY SPONSORED RESEARCH OR DEVELOPMENT

This invention was made with government support under Grant No. R21 AI149694 awarded by the National Institutes of Health (NIH). The government has certain rights in the invention.

### SEQUENCE LISTING

The instant application contains a Sequence Listing which has been submitted electronically in XML format and is hereby incorporated by reference in its entirety. The xml copy, created on May 3, 2024, is named 753652_083474-015DV_ST26.xml and is 950,786 bytes in size.

### TECHNOLOGY FIELD

The subject matter disclosed herein is generally related to systems, methods, and compositions for RNA-guided RNA-targeting CRISPR effectors for the treatment of diseases and diagnostics.

### BACKGROUND

RNA targeting tools for genetic engineering are important to study RNA biology and develop RNA therapeutics. These tools can regulate intracellular and intercellular target-gene functions and expressions and manipulate specific target-genomic information. In contrast to DNA targeting tools, only a few RNA targeting tools for CRISPR applications have been developed, and these RNA specific tools present challenges. For instance, protein complexes for CRISPR RNA editing can have a weak activity in mammalian cells and present collateral effects which can be toxic in some cell types. Also, the size of the RNA editing tools can be a significant barrier to their use, as technologies such as programmable activation cannot be delivered in mouse models using common methods such as adeno-associated vectors (AAV). Development of effective gene and cell therapies requires genome editing tools and delivery technologies that can meet the demands for cell type specificity, large payload sizes, and efficient integration of diverse and large sequences. Many applications, including single-vector homology directed repair (HDR), CRISPR base editing, gene activation, and large gene delivery, are limited by AAV size restrictions. Thus, there remains a need for more effective tools for gene correction and delivery.

### SUMMARY

The present invention is directed to systems, methods, and compositions for RNA-guided RNA-targeting CRISPR effectors for the treatment of diseases and diagnostics. It also pertains to nucleotide deaminase functionalized CRISPR systems for RNA editing RNA knockdown, viral resistance, splicing modulation, RNA tracking, translation modulation, and epi-transcriptomic modifications.

In one aspect, a composition that cleaves an RNA target is discussed, which comprises a guide RNA that specifically hybridizes to the RNA target, and a polypeptide comprising an amino acid sequence 85% identical to an amino acid sequence selected from the group consisting of SEQ ID NOs: 1-32 and 632. The amino acid sequence can be SEQ ID NO: 1. The polypeptide can comprise a glutamate at a position corresponding to position 70 of SEQ ID NO:1, an aspartate at a position corresponding to position 429 of SEQ ID NO:1, an aspartate at a position corresponding to position 487 of SEQ ID NO:1, and/or an aspartate at a position corresponding to position 654 of SEQ ID NO:1. If desired, the polypeptide can comprise an amino acid sequence 90% identical to the amino acid sequence of SEQ ID NO:1. If desired, the polypeptide can comprise an amino acid sequence 95% identical to the amino acid sequence of SEQ ID NO:1. If desired, the polypeptide can comprise an amino acid sequence 99% identical to the amino acid sequence of SEQ ID NO:1. If desired, the polypeptide can be the amino acid sequence of SEQ ID NO:1.

Yet in another aspect, the guide RNA comprises a mismatch distance that is 20-65% of the length of the guide. If desired, the guide RNA can comprise a mismatch that is about 20 to about 30 nucleotides from the non-pairing C of the guide RNA. If desired, the guide RNA can have a sequence with a length of from about 20 to about 53 nucleotides (nt), preferably from about 25 to about 53 nt, more preferably from about 29 to about 53 nt or from about 40 to about 50 nt. If desired, the guide RNA can be a pre-crRNA. If desired, the guide RNA can be a mature crRNA.

Yet in another aspect, the RNA target can be a single-strand RNA (ssRNA). If desired, the RNA target can be in a cell. The cell can be a prokaryotic cell. The cell can be a eukaryotic cell. The eukaryotic cell can be a mammalian cell. The mammalian cell can be a human cell.

Yet in another aspect, the polypeptide can comprise a deaminase domain. The deaminase can be selected from the group consisting of an adenosine deaminase, a cytidine deaminase, and a catalytic domain thereof. If desired, the deaminase can be an adenosine deaminase. If desired, the deaminase domain can comprise an amino acid sequence 85% identical to an amino acid sequence selected from the group consisting of SEQ ID NOs: 25-31. If desired, the deaminase domain can comprise an amino acid sequence 85% identical to an amino acid sequence selected from the group consisting of SEQ ID NOs: 32-42.

Yet in another aspect, a nucleic acid molecule encoding the polypeptide is discussed. The nucleic acid molecule can encode the guide RNA.

Yet in another aspect, a vector comprising the nucleic acid molecule is discussed.

Yet in another aspect, a cell comprising the composition, the nucleic acid molecule or the vector is discussed. If desired, the cell can be a prokaryotic cell. If desired, the cell can be a eukaryotic cell. The eukaryotic cell can be a mammalian cell. The mammalian cell can be a human cell.

Yet in another aspect, a method of cleaving an RNA target in a cell is discussed, which comprises providing to the cell the composition, the nucleic acid molecule or the vector. If desired, the RNA target can be an ssRNA.

Yet in another aspect, a method of stabilizing an RNA target in a cell is discussed, which comprises providing to the cell the composition, the nucleic acid molecule or the vector. If desired, the RNA target can be an ssRNA.

Yet in another aspect, a method of affecting translation of an RNA target in a cell is discussed, which comprises providing to the cell the composition, the nucleic acid molecule or the vector. If desired, the RNA target can be an ssRNA.

Yet in another aspect, a method of treating a genetically inherited disease in a subject in need thereof is discussed, which comprises administering to the subject an effective amount of the composition, the nucleic acid molecule or the vector, wherein the genetically inherited disease involves a guanosine to adenosine change in the genome of the subject. The genetically inherited disease can be selected from the group consisting of Meier-Gorlin syndrome; Seckel syndrome 4; Joubert syndrome 5; Leber congenital amaurosis 10; Charcot-Marie-Tooth disease, type 2; leukoencephalopathy; Usher syndrome, type 2C; spinocerebellar ataxia 28; glycogen storage disease type III; primary hyperoxaluria, type I; long QT syndrome 2; Sjögren-Larsson syndrome; hereditary fructosuria; neuroblastoma; amyotrophic lateral sclerosis type 9; Kallmann syndrome 1; limb-girdle muscular dystrophy, type 2L; familial adenomatous polyposis 1; familial type 3 hyperlipoproteinemia; Alzheimer's disease, type 1; metachromatic leukodystrophy; and cancer.

Yet in another aspect, a method of treating a genetically inherited disease in a subject in need thereof is discussed, which comprises administering to the subject an effective amount of the composition, the nucleic acid molecule or the vector, wherein the genetically inherited disease is a pre-termination disease.

Yet in another aspect, a method of altering splicing of a pre-mRNA in a cell is discussed, which comprises administering to the cell an effective amount of the composition, the nucleic acid molecule or the vector.

Yet in another aspect, a method of changing microRNA targets in a subject in need is discussed, which comprises administering to the subject an effective amount of the composition, the nucleic acid molecule or the vector.

Yet in another aspect, a method of increasing RNA stability in a cell is discussed, which comprises administering to the cell an effective amount of the composition, the nucleic acid molecule or the vector.

Yet in another aspect, a method of modulating translation in a cell is discussed, which comprises administering to the cell an effective amount of the composition, the nucleic acid molecule or the vector.

Yet in another aspect, a method of detecting a bacterium or derivative thereof in a sample is discussed, the method comprises adding to the sample an effective amount of the composition, the nucleic acid molecule or the vector, and detecting a reporter specific to the bacterium or derivative thereof.

Yet in another aspect, a method of detecting a virus or derivative thereof in a sample is discussed, the method comprises adding to the sample an effective amount of the composition, the nucleic acid molecule or the vector, and detecting a reporter specific to the virus or derivative thereof.

These and other aspects of the applicants' teaching are set forth herein.

### BRIEF DESCRIPTION OF THE DRAWINGS

Aspects, features, benefits and advantages of the embodiments described herein will be apparent with regard to the following description, appended claims, and accompanying drawings where:

FIG. 1A is a schematic representation of RNA editing via dDisCas7-11a-NES-ADAR2 fusions that are programmed to target a specific adenosine via a cytidine mismatch for A to I editing in the guide:target RNA duplex according to embodiments of the present teachings;

FIG. 1B is a schematic of Cas7-11 expression, processing, and interference against ssRNA viruses and other ssRNA targets according to embodiments of the present teachings;

FIG. 1C illustrates the comparison of Cas7-11 features to characteristics of Cas13 and type III-A/B/C systems according to embodiments of the present teachings;

FIG. 1D illustrates hypothetical scenario for the evolution of a single-protein CRISPR-Cas effector from a multi-subunit effector complex according to embodiments of the present teachings;

FIG. 1E shows type III-E family members and type III-D2 loci architecture and multiple alignment of representative orthologs, wherein CRISPR array symbols are not representative of the number of spacers in the array according to embodiments of the present teachings;

FIG. 1F illustrates multiple alignments of representative Cas7-11 orthologs showing conservation of the residues involved in catalysis (D177) according to embodiments of the present teachings. Figure discloses SEQ ID NOS 451-475, respectively, in order of appearance;

FIG. 1G illustrates multiple alignments of representative Cas7-11 orthologs showing conservation of the residues involved in catalysis (D429) according to embodiments of the present teachings. Figure discloses SEQ ID NOS 476-502, respectively, in order of appearance;

FIG. 1H illustrates multiple alignments of representative Cas7-11 orthologs showing conservation of the residues involved in catalysis (D654) according to embodiments of the present teachings. Figure discloses SEQ ID NOS 503-529, respectively, in order of appearance;

FIG. 1I illustrates multiple alignments of representative Cas7-11 orthologs showing conservation of the residues involved in catalysis (D745, D758) according to embodiments of the present teachings. Figure discloses SEQ ID NOS 530-556, respectively, in order of appearance;

FIG. 1J illustrates multiple alignments of representative Cas7-11 orthologs showing conservation of the residues involved in catalysis (E959) according to embodiments of the present teachings. Figure discloses SEQ ID NOS 557-583, respectively, in order of appearance;

FIG. 1K illustrates multiple alignments of representative Cas7-11 orthologs showing conservation of the residues involved in catalysis (E998) according to embodiments of the present teachings. Figure discloses SEQ ID NOS 584-610, respectively, in order of appearance;

FIG. 2A illustrates tree of type III-D2 and III-E effectors based on Cas7-11 and Cas7x3 multiple alignment via a computational discovery pipeline according to embodiments of the present teachings;

FIG. 2B is a schematic of a typical type III-E Cas7-11 locus and type III-D2 Cas7x3 locus wherein the inset shows the multiple Cas7-like domains of Cas7-11 including a putative small subunit Cas11 domain and an uncharacterized insertion in the Cas7-like domain 5 according to embodiments of the present teachings;

FIG. 2C illustrates a native expression of a CRISPR array associated with DisCas7-11 according to embodiments of the present teachings. Figure discloses SEQ ID NO: 633;

FIG. 2D is a schematic representation of a DR secondary structure and a scissor denoting the cleavage site for processing and maturation according to embodiments of the present teachings. Figure discloses SEQ ID NO: 634;

FIG. 3A illustrates the CRISPR array screen of RNA-guided RNA-targeting Cas7-11 for all crRNAs targeting the MS2 phage genome according to embodiments of the present teachings;

FIG. 3B is a diagram showing results of the MS2 interference screen (for the MAS targeting spacers) according to embodiments of the present teachings;

FIG. 3C is a diagram showing results of the MS2 interference screen (for the non-targeting spacers) according to embodiments of the present teachings;

FIG. 3D are images of plaque dilution assays for MS2 targeting spacer and non-targeting spacer according to embodiments of the present teachings;

FIG. 3E is a diagram of the number of DisCas7-11a spacers that display survival enrichment over a threshold of 1.7 across different phage dilution conditions according to embodiments of the present teachings;

FIG. 3F is a diagram showing Quantification of resistance conferred by top MS2-targeting DisCas7-11 spacers compared against a panel of 4 non-targeting spacers according to embodiments of the present teachings;

FIG. 3G is a diagram showing the quantification of resistance conferred by two MS2-targeting DisCas7-11 spacers compared against a panel of 6 non-targeting spacers according to embodiments of the present teachings;

FIG. 3H illustrates the RFP knockdown assay containing a DisCas7-11 and spacers expression vector co-transformed with an RFP-expressing vector according to embodiments of the present teachings;

FIG. 3I is a diagram of the DisCas7-11-mediate RFP knockdown by multiple targeting spacers normalized to a non-targeting condition as determined by flow cytometry according to embodiments of the present teachings;

FIG. 3J is a schematic of the DisCas7-11 a full locus with TPR-CHAT nearby and DisCas7-11 a effector expressed alone with TPR-CHAT according to embodiments of the present teachings;

FIG. 3K is a diagram of the MS2 interference activity of DisCas7-11a in the full locus and as a single effector with and without Csx29 expression according to embodiments of the present teachings;

FIG. 4A illustrates a CRISPR array processing assay for DisCas7-11 according to embodiments of the present teachings;

FIG. 4B illustrates the in vitori cleavage of ssRNA 1 target according to embodiments of the present teachings;

FIG. 4C illustrates the cleavage of EGFP ssRNA at multiple sites with tiled crRNAs according to embodiments of the present teachings;

FIG. 4D illustrates the protospacer flanking site (PFS) sequence screen showing in vitro cleavage of randomized PFS targets and lack of sequence preference flanking the target site for DisCas7-11 cleavage according to embodiments of the present teachings. Figure discloses SEQ ID NOS 635-636, respectively, in order of appearance;

FIG. 4E is a diagram of the kinetics of fluorescence due to RnaseAlert collateral cleavage by DisCas7-11 or LwaCas13a targeting and non-targeting guides against the MS2 RNA target according to embodiments of the present teachings;

FIG. 4F illustrates the processing activity of a synthetic DisCas7-11 CRISPR array by DisCas7-11 protein with predicted catalytic processing mutants in the protein insert region with a characteristic K×Y×H catalytic according to embodiments of the present teachings;

FIG. 4G is the graph showing the measurement of bacterial growth (OD600) during target interference by DisCas7-11 and LwaCas13 according to embodiments of the present teachings;

FIG. 5A is a schematic of the protein domains of DisCas7-11 with putative catalytic residues highlighted in Cas7-like domains 3 and 4 according to embodiments of the present teachings;

FIG. 5B illustrates that the cleavage of synthetic ssRNA target with two cRNAs and wild type, D429A, D654A, and D429A/D654A DisCas7-11 proteins according to embodiments of the present teachings;

FIG. 5C illustrates the processing of a synthetic DisCas7-11 CRISPR array by catalytic DisCas7-11 mutants according to embodiments of the present teachings;

FIG. 5D is a diagram showing the MS2 phage interference in bacteria according to embodiments of the present teachings;

FIG. 5E is a diagram showing the RFP knockdown in E. coli that is prevented by the catalytic mutation D429A/D654A in DisCas7-11 according to embodiments of the present teachings;

FIG. 5F illustrates the DisCas7-11:crRNA complex binding to a complementary MS2 ssRNA target that is determined by electrophoretic mobility shift assay (EMSA) according to embodiments of the present teachings;

FIG. 6A illustrates DisCas7-11 guides targeting multiple sites on a synthetic 100 nt EGFP ssRNA according to embodiments of the present teachings;

FIG. 6B are images of cleavage patterns of each EGFP-targeting guide incubated with DisCas7-11 on a 3′ labeled EGFP ssRNA according to embodiments of the present teachings;

FIG. 6C illustrates DisCas7-11 that is incubated with a panel of crRNAs targeting either MS2 ssRNA or ssRNA target 2, two targets of equivalent length (153 nt) according to embodiments of the present teachings;

FIG. 6D are images of cleavage patterns of each guide incubated with MS2 ssRNA or ssRNA target 2 according to embodiments of the present teachings;

FIG. 7A tbc is a schematic of three Cas7-11 orthologs (GwCas7-11, CjcCas7-11, and DisCas7-11) according to embodiments of the present teachings;

FIG. 7B illustrates multiple sequence alignments of the DR sequences for GwCas7-11, CjcCas7-11, and DisCas7-11 according to embodiments of the present teachings. Figure discloses SEQ ID NOS 637, 638, and 634, respectively, in order of appearance;

FIG. 7C illustrates the activity of Cas7-11 from Candidatus Jettenia caeni (CjcCas7-11) according to embodiments of the present teachings;

FIG. 7D illustrates the activity of Cas7-11 from groundwater metagenomes (GwCas7-11) according to embodiments of the present teachings;

FIG. 7E illustrates the MS2 ssRNA target cleavage by GwCas7-11 and CjcCas7-11 incubated with their cognate crRNAs complementary to the MS2 ssRNA target according to embodiments of the present teachings;

FIG. 7F illustrates the incubation of GwCas7x3 and CjcCas7-11 proteins with their cognate crRNAs complementary to the MS2 ssRNA target at varying positions along the target according to embodiments of the present teachings;

FIG. 8A is a schematic of a luciferase reporter assay for RNA knockdown using a vector expressing human codon-optimized DisCas7-11 and guide RNA according to embodiments of the present teachings;

FIG. 8B is a diagram of the knockdown activity of DisCas7-11 against the Gaussia luciferase (Gluc) transcript normalized to two non-targeting conditions according to embodiments of the present teachings;

FIG. 8C is a diagram showing the knockdown activity of DisCas7-11 against the multiple endogenous transcripts normalized to two non-targeting according to embodiments of the present teachings;

FIG. 8D is a schematic of the DisCas7-11a guide design for programmable A-to-I editing according to embodiments of the present teachings. Figure discloses SEQ ID NOS 639-640, respectively, in order of appearance;

FIG. 8E is a diagram showing the RNA A-to-I editing of Cypridina luciferase (Cluc) mRNA W85X mutation in mammalian cells by dead DisCas7-11a-NES-ADAR2 according to embodiments of the present teachings;

FIG. 8F is a diagram showing the RNA A-to-I editing of Cypridina luciferase (Cluc) mRNA W85X mutation in mammalian cells by dead DisCas7-11a-NES-ADAR2 according to embodiments of the present teachings;

FIG. 8G is a graph illustrating the RFP knockdown by DisCas7-11 or LwaCas13a in E. coli bacteria according to embodiments of the present teachings;

FIG. 8H is a bar graph illustrating the knockdown of Gluc mRNA in mammalian cell by DisCas7-11 with a panel of guides containing the mature DR sequence according to embodiments of the present teachings;

FIG. 8I is a bar graph illustrating knockdown of Gluc mRNA in mammalian cells by DisCas7-11 with a panel of guides of different lengths according to embodiments of the present teachings;

FIG. 8J is a bar graph comparing the number of significant off-targets when targeting the Gluc transcript in HEK293 FT and U87 cells by DisCas7-11, shRNA, LwaCas13a, PspCas13b, and RfxCas13d using transcriptome-wide RNA-sequencing data according to embodiments of the present teachings;

FIG. 8K is a bar graph comparing the number of significant off-targets when targeting the Gluc transcript in HEK293 FT and U87 cells by DisCas7-11, shRNA, LwaCas13a, PspCas13b, and RfxCas13d using transcriptome-wide RNA sequencing data according to embodiments of the present teachings;

FIG. 8L is a bar graph showing the knockdown activity in HEK293 FT cells of DisCas7-11, shRNA, LwaCas13a, PspCas13b, and RfxCas13d against the Gaussia luciferase (Gluc) transcript normalized to corresponding non-targeting controls according to the embodiments of the present teachings;

FIG. 8M shows the transcriptome wide specificity in HEK293 FT cells of RNA knockdown by DisCas7-11, RfxCas13d, and shRNA against the Gaussia luciferase (Gluc) transcript, wherein the analysis off targets is performed via significance testing (y-axis) of differentially expressed transcripts (x-axis) between targeting and non-targeting guide conditions (n=3) and the number of significant off-targets is displayed according to embodiments of the present teachings;

FIG. 8N is a bar graph showing the cell viability of HEK293 FT cells during RNA knockdown by DisCas7-11, shRNA, LwaCas13a, PspCas13b, and RfxCas13d against the Gaussia luciferase (Gluc) transcript according to embodiments of the present teachings;

FIG. 8O is a bar graph of the knockdown activity in U87 glioblastoma cells of DisCas7-11, shRNA, LwaCas13a, PspCas13b, and RfxCas13d against the Gaussia luciferase (Gluc) transcript normalized to corresponding non-targeting controls according to embodiments of the present teachings;

FIG. 8P is showing the transcriptome wide specificity in U87 glioblastoma cells of RNA knockdown by DisCas7-11, PspCas13b, and RfxCas13d against the Gaussia luciferase (Gluc) transcript, wherein the analysis off targets is performed via significance testing (y-axis) of differentially expressed transcripts (x-axis) between the targeting guide condition and cells only expressing EGFP (n=3) and the number of significant off-targets is displayed according to embodiments of the present teachings;

FIG. 8Q is a graph of the cell viability of U87 glioblastoma cells during RNA knockdown by DisCas7-11, shRNA, LwaCas13a, PspCas13b, and RfxCas13d against the Gaussia luciferase (Gluc) transcript, wherein the data are mean (n=3)±s.e.m. according to embodiments of the present teachings;

FIG. 8R is a graph showing the knockdown activity in HEK293 FT cells of DisCas7-11, LwaCas13a, PspCas13b, and RfxCas13d against the KRAS transcript normalized to corresponding non-targeting controls according to embodiments of the present teachings;

FIG. 8S is a graph showing the knockdown activity in HEK293 FT cells of DisCas7-11, LwaCas13a, PspCas13b, and RfxCas13d against the PPIB transcript normalized to corresponding non-targeting controls according to embodiments of the present teachings;

FIG. 8T is a graph showing the knockdown activity in HEK293 FT cells of DisCas7-11, LwaCas13a, PspCas13b, and RfxCas13d against the MALAT transcript normalized to corresponding non-targeting controls according to embodiments of the present teachings;

FIG. 8U is a schematic of type III-E loci from Hydrothermal vent microbial metagenome sample, Hydrothermal sediment microbial communities metagenome sample, and Deltaproteobacteria bacterium, containing HvsCas7-11, HsmCas7-11, and DpbaCas7-11 respectively according to embodiments of the present teachings;

FIG. 8V is a graph showing the knockdown activity in HEK293 FT cells of HvsCas7-11, HsmCas7-11, DpbaCas7-11, and DisCas7-11 against the Gaussia luciferase (Gluc) transcript normalized to corresponding non-targeting controls, wherein for HvsCas7-11, HsmCas7-11, and DpbaCas7-11, two DR orientations (“F” and “R”) were tested, and wherein significance tests are measured by a Student's t-test (*, p-value<0.05; **, p-value<0.01) according to embodiments of the present teachings;

FIG. 8W is a graph showing the knockdown activity in HEK293 FT cells of HvsCas7-11 and DisCas7-11 with two distinct target guides, with and without catalytic mutations in the Cas7-11 proteins according to embodiments of the present teachings;

FIG. 8X is a graph showing the knockdown activity in HEK293 FT cells of HvsCas7-11 against the PPIB transcript normalized to corresponding non-targeting controls according to embodiments of the present teachings;

FIG. 8Y is a graph showing the knockdown activity in HEK293 FT, HepG2, and primary neuron cultures of HvsCas7-11 delivered as a two vector AAV system targeting the Gaussia luciferase (Gluc) transcript, wherein values are normalized to corresponding non-targeting controls, and wherein significance tests are measured by a Student's t-test (*, p-value<0.05; **, p-value<0.01) according to embodiments of the present teachings;

FIG. 8Z is a graph showing the knockdown activity in mouse embryonic stem cells (ESCs) of DisCas7-11, HvsCas7-11, PspCas13b, and RfxCas13d against the Gaussia luciferase (Gluc) transcript normalized to corresponding non-targeting controls according to embodiments of the present teachings;

FIG. 8AA is a graph showing the ribosomal RNA integrity analysis measured by BioAnalyzer traces in U87 cells treated with DisCas7-11, shRNA, LwaCas13a, and PspCas13b targeting Gluc transcripts for knockdown (the normalized area under the curves for the 18S and 28S bands) according to embodiments of the present teachings;

FIG. 8AB are graph showing the comparison of DisCas7-11 and RfCas13d knockdown activity across multiple guides in HepG2 cells along with data on collateral activity as measured by Cluc levels, cell health (live stain), and cell toxicity (dead stain), wherein data are mean (n=3)±s.e.m according to embodiments of the present teachings;

FIG. 8AC is a graph showing the collateral activity in mouse embryonic stem cells as measured by Cluc levels during Gluc RNA knockdown by DisCas7-11, HvsCas7-11, PspCas13b, and RfxCas13d, wherein the asterisks indicate significant as measured by a Student's t-test between the targeting and non-targeting conditions of a listed ortholog condition (**, p-value<0.01), and wherein data are mean (n=3)±s.e.m according to embodiments of the present teachings;

FIG. 9A illustrates the Desulfonema ishimotonii Type III-E full locus matures crRNAs in E. coli according to embodiments of the present teachings;

FIG. 9B illustrates the single effector protein DisCas7-11 matures crRNAs in E. coli according to embodiments of the present teachings;

FIG. 9C illustrates the expression of the DisCas7-11 CRISPR array without any effector or accessory proteins according to embodiments of the present teachings;

FIG. 9D illustrates types III-E and III-D2 loci containing Cas7-11 and Cas7x3 respectively according to embodiments of the present teachings;

FIG. 10 are images of MS2 plaque dilution assays for MS2 targeting spacers, non-targeting spacers and RFP targeting spacers with DisCas7-11 according to embodiments of the present teachings;

FIG. 11A illustrates the Type III-E locus of the Desulfonema ishimotonii according to embodiments of the present teachings;

FIG. 11B is a diagram showing the phage plaque assay of the Type III-E DisCas7-11 CRISPR-Cas locus according to embodiments of the present teachings;

FIG. 11C is a diagram showing the phage plaque assay of the individual gene knock-in together with of the Type III-E DisCas7-11 CRISPR-Cas system according to embodiments of the present teachings;

FIG. 12A illustrates the in vitro transcription of pre-crRNA and processing by DisCas7-11 according to embodiments of the present teachings;

FIG. 12B is a sequence of a single spacer CRISPR array showing locations of mutated bases according to embodiments of the present teachings. Figure discloses SEQ ID NO: 641;

FIG. 12C is an image of the processing of transcribed pre-crRNA showing the effect of single position mutations on processing according to embodiments of the present teachings;

FIG. 12D is an image of the processing of transcribed pre-crRNA showing the effect of consecutive double mutations on processing according to embodiments of the present teachings;

FIG. 13A illustrates the processing of pre-crRNA by DisCas7-11 in the presence of different ions or chelating agents according to embodiments of the present teachings;

FIG. 13B is a schematic of in vitro cleavage of ssRNA target with DisCas7-11 and crRNA 5 according to embodiments of the present teachings;

FIG. 13C illustrates the incubation of ssRNA target with DisCas7-11 and targeting or non-targeting crRNA in the presence of different ions or chelating agents according to embodiments of the present teachings;

FIG. 13D illustrates the cleavage of ssRNA target with increasing amounts of DisCas7-11-crRNA complex from 0 nM to 233 nM according to embodiments of the present teachings;

FIG. 13E illustrates the cleavage of ssRNA target at increasing incubation periods from 0 minutes to 180 minutes according to embodiments of the present teachings;

FIG. 14A is a schematic showing sequence of DisCas7-11 crRNA 5, targeting the ssRNA target according to embodiments of the present teachings. Figure discloses SEQ ID NO: 642;

FIG. 14B illustrates the cleavage of ssRNA target with crRNA of varying DR and spacer lengths according to embodiments of the present teachings;

FIG. 14C is a sequence of DisCas7-11 crRNA 1 targeting the MS2 target according to embodiments of the present teachings. Figure discloses SEQ ID NO: 643;

FIG. 14D illustrates the cleavage of the MS2 target according to embodiments of the present teachings;

FIG. 15 is a diagram of the fluorescence of RnaseAlert reporter incubated with either DisCas7-11 or LwaCas13a targeting and non-targeting guides against MS2 ssRNA target according to embodiments of the present teachings;

FIG. 16A is a schematic showing positions of DisCas7-11 crRNA 1 and crRNA 2 targeting the MS2 target according to embodiments of the present teachings;

FIG. 16B illustrates the cleavage of a synthetic MS2 ssRNA target with two cRNAs and wild type, D429A, D654A, and D429A/D654A DisCas7-11 proteins according to embodiments of the present teachings;

FIG. 17A illustrates an EMSA assay for targeting crRNA in the absence of DisCas7-11 according to embodiments of the present teachings;

FIG. 17B illustrates the quantification of band intensities for EMSA gels according to embodiments of the present teachings;

FIG. 17C illustrates the quantification of band intensities for EMSA gels according to embodiments of the present teachings;

FIG. 17D illustrates the quantification of band intensities for EMSA gels according to embodiments of the present teachings;

FIG. 17E illustrates the quantification of band intensities for EMSA gels according to embodiments of the present teachings;

FIG. 17F illustrates the quantification of band intensities for EMSA gels according to embodiments of the present teachings;

FIG. 18A illustrates DisCas7-11 incubated with a panel of crRNAs targeting either MS2 ssRNA or ssRNA target 2, two targets of equivalent length (153 nt) according to embodiments of the present teachings;

FIG. 18B illustrates the cleavage of ssRNA 1 and MS2 ssRNA at multiple sites with tiled crRNAs, showing a varying cleavage pattern based on target position according to embodiments of the present disclosures;

FIG. 18C shows the schematic of the position of the tested crRNAs along the SARS-CoV-2 ssRNA and the results from the cleavage of synthetic SARS-CoV-2 ssRNA at multiple sites with tiled crRNAs showing a varying cleavage pattern based on target position according to embodiments of the present teachings;

FIG. 18D shows the schematic of the position of the tested crRNAs along the Influenza A ssRNA target and the results from the cleavage of synthetic influenza A segment 4 HA gene ssRNA at multiple sites with tiled crRNAs according to embodiments of the present teachings;

FIG. 18E illustrates DisCas7-11a incubated with a crRNA targeting a crRNA site flanked by different sequences according to embodiments of the present teachings;

FIG. 18F illustrates DisCas7-11a incubated with a crRNA targeting a double-stranded (dsDNA) target with 5′ labeling of the bottom strand according to embodiments of the present teachings;

FIG. 18G illustrates DisCas7-11a incubated with a crRNA targeting a double-stranded (dsDNA) target with 5′ labeling of the top strand according to embodiments of the present teachings;

FIG. 19A shows the schematic of the position of the tested crRNAs along the 100 nt EGFP ssRNA target and the biochemical characterization of programmable DisCas7-11 RNA cleavage of 100 nt long and 3′ fluorescently labeled EGFP ssRNA target with tiling crRNAs according to embodiments of the present teachings;

FIG. 19B shows the schematic of the position of the tested crRNAs along the 200 nt EGFP ssRNA target and the biochemical characterization of the programmable DisCas7-11 RNA cleavage of 200 nt long and 3′ fluorescently labeled EGFP ssRNA target with tiling crRNAs according to embodiments of the present teachings;

FIG. 19C shows the schematic of the position of the tested crRNAs along the 600 nt EGFP ssRNA target and the biochemical characterization of the programmable DisCas7-11 RNA cleavage of 600 nt long and 3′ fluorescently labeled EGFP ssRNA target with tiling crRNAs according to embodiments of the present teachings;

FIG. 19D shows the DNA sequencing analysis of selected EGFP targeting crRNA conditions on an unlabeled 400 nt EGFP ssRNA target according to embodiments of the present teachings;

FIG. 19E shows the in vitro cleavage of ssRNA 1 at 37° C. with DisCas7-11 incubated with a crRNA against the 60 nt MS2 target labeled with a 5′ or 3′ Cy5 molecule, wherein the target cleavage is assessed with and without crRNA, DisCas7-11 protein, and Mg+2, and arrows on the target schematic show estimated cleavage positions of DisCas7-11 cleavage according to embodiments of the present teachings;

FIG. 19F shows cleavage of MS2 ssRNA at multiple sites with tiled crRNAs via incubation with DisCas7-11 protein, wherein MS2 ssRNA target is 5′ labeled with Cy5 according to embodiments of the present teachings; and

FIG. 19G shows in vitro cleavage of MS2 ssRNA at 37° C. with varying concentrations of DisCas7-11 incubated with a crRNA against the target with and without Csx29 protein, wherein MS2 ssRNA target is 5′ labeled with Cy5 and cleavage bands are marked by asterisks according to embodiments of the present teachings.

FIG. 20 illustrates the heterologous expression of the Candidatus Jettenia caeni Type III-E CRISPR-Cas system and associated CRISPR array in E. coli. according to embodiments of the present teachings. Figure discloses SEQ ID NO: 644;

FIG. 21A illustrates the processing of DisCas7-11 ortholog according to embodiments of the present teachings;

FIG. 21B illustrates the processing of GwCas7-11 ortholog according to embodiments of the present teachings.

FIG. 21C illustrates the processing of CjcCas7-11 ortholog according to embodiments of the present teachings;

FIG. 21D illustrates a comparison of target cleavages between active CjcCas7-11 and dead CjcCas7-11 according to embodiments of the present teachings;

FIG. 22A illustrates the DisCas7-11 guide design for RNA editing according to embodiments of the present teachings. Figure discloses SEQ ID NOS 639-640, respectively, in order of appearance;

FIG. 22B is a table showing the DisCas7-11 ADAR guide designs for according to embodiments of the present teachings. Figure discloses SEQ ID NOS 425-446, 645-647, and 411, respectively, in order of appearance;

FIG. 22C is diagram showing the dDisCas7-11 correction of Cluc W85X RNA according to embodiments of the present teachings;

FIG. 22D is a diagram showing the dDisCas7-11 correction of RNA as measured by sequencing according to embodiments of the present teachings;

FIG. 22E is a diagram showing the luciferase correction that correlates to the correction percent according to embodiments of the present teachings;

FIG. 23A illustrates the modulation of protein function that requires temporal A to G modulation according to embodiments of the present teachings;

FIG. 23B illustrates the correction of mendelian disease that requires temporal A to G modulation according to embodiments of the present teachings;

FIG. 23C illustrates a slicing modulation that requires temporal A to G modulation according to embodiments of the present teachings;

FIG. 24A illustrates the initial and edited codon for protein modifications with A to G RNA editing according to embodiments of the present teachings;

FIG. 24B illustrates first, second and third position codon modifications in RNA editing according to embodiments of the present teachings;

FIG. 25 illustrates the initial codon, final codon and potential function in protein modifications with C to U RNA editing according to embodiments of the present teachings;

FIG. 26A illustrates the correction of mendelian disease that requires C to U RNA editing according to embodiments of the present teachings;

FIG. 26B illustrates the multiplexed creation of disease-protective alleles that requires C to U RNA editing according to embodiments of the present teachings;

FIG. 26C illustrates the modulation of catalytic activity that requires C to U RNA editing according to embodiments of the present teachings;

FIG. 26D illustrates alter post-translation modifications that requires C to U RNA editing according to embodiments of the present teachings;

FIG. 27A-27C are tables of Cas7-11 effectors according to embodiments of the present teachings;

FIG. 28A illustrates the targeting of DisCas7-11 RNA target in beta-lactamase (ampicillin resistance gene) and the resulting PFS determined by 20 depleted targets according to embodiments of the present teachings;

FIG. 28B illustrates the targeting of DisCas7-11 DNA target in a plasmid, with a motif corresponding to no significantly depleted PFS sequences according to embodiments of the present teachings;

FIG. 28C is a diagram showing the PFS analysis of top spacers from the MS2 phage screen (e-5 condition) in the 8 bp flanking the target region to the left according to embodiments of the present teachings;

FIG. 28D is a diagram showing the PFS analysis of top spacers from the MS2 phage screen (e-5 condition) in the 8 bp flanking the target region to the right according to embodiments of the present teachings;

FIG. 28E is a schematic of the heterologous type III-E locus of the Desulfonema ishimotonii according to embodiments of the present teachings;

FIG. 28F is a bar graph showing the phage plaque assay of the Type III-E DisCas7-11 effector alone and as part of it entire locus according to embodiments of the present teachings;

FIG. 28G is a bar graph showing the phage plaque assay of E. coli transformed with the DisCas7-11 locus containing according to embodiments of the present teachings;

FIG. 29 illustrates the DisCas7-11a cleavage of synthetic 31 nt MS2 ssRNA with a 31 nt crRNA according to embodiments of the present teachings;

FIG. 30A shows the secondary structure folding prediction of the DisCas7-11a DR according to embodiments of the present teachings. Figure discloses SEQ ID NO: 634;

FIG. 30B shows the secondary structure folding prediction of the CjcCas7-11b DR according to embodiments of the present teachings. Figure discloses SEQ ID NO: 638;

FIG. 30C shows the structure folding prediction of the GwCas7-11c DR in accordance with embodiments of the present teachings. Figure discloses SEQ ID NO: 637;

FIG. 31A illustrates the comparison of knockdown activity of Gluc mRNA in mammalian cells between active DisCas7-11a, catalytically inactive D429A/D654A DisCas7-11a, and GFP according to embodiments of the present teachings;

FIG. 31B illustrate the comparison of knockdown activity of endogenous mRNA in mammalian cells between active DisCas7-11a, catalytically inactive D429A/D654A DisCas7-11a, and GFP according to embodiments of the present teachings;

FIG. 32A is a diagram of the RNA A-to-I editing of Cypridinia luciferase (cluc) mRNA W85X mutation in mammalian cells by active DisCas7-11a-NES-ADAR2 or dead DisCas7-11a-NES-ADAR2 according to embodiments of the present teachings;

FIG. 32B is a diagram of the RNA A-to-I editing of Cypridinia luciferase (cluc) mRNA W85X mutation in mammalian cells by active DisCas7-11a-NES-ADAR2 or dead DisCas7-11a-NES-ADAR2 according to embodiments of the present teachings;

FIG. 33A shows the cleavage of 3′labeled targeted by incubation with RNase H according to embodiments of the present teachings;

FIG. 33B shows the cleavage of 5′labeled targeted by incubation with RNase H according to embodiments of the present teachings;

FIG. 34A shows domains and section 1 of sequence alignments of Cas nucleases according to embodiments of the present teachings. FIGS. 34A-O disclose SEQ ID NOS 611-631, respectively, in order of appearance;

FIG. 34B shows section 2 of sequence alignments of Cas nucleases according to embodiments of the present teachings. FIGS. 34A-O disclose SEQ ID NOS 611-631, respectively, in order of appearance;

FIG. 34C shows section 3 of sequence alignments of Cas nucleases according to embodiments of the present teachings. FIGS. 34A-O disclose SEQ ID NOS 611-631, respectively, in order of appearance;

FIG. 34D shows section 4 of sequence alignments of Cas nucleases according to embodiments of the present teachings. FIGS. 34A-O disclose SEQ ID NOS 611-631, respectively, in order of appearance;

FIG. 34E shows section 5 of sequence alignments of Cas nucleases according to embodiments of the present teachings. FIGS. 34A-O disclose SEQ ID NOS 611-631, respectively, in order of appearance;

FIG. 34F shows section 6 of sequence alignments of Cas nucleases according to embodiments of the present teachings. FIGS. 34A-O disclose SEQ ID NOS 611-631, respectively, in order of appearance;

FIG. 34G shows section 7 of sequence alignments of Cas nucleases according to embodiments of the present teachings. FIGS. 34A-O disclose SEQ ID NOS 611-631, respectively, in order of appearance;

FIG. 34H shows section 8 of sequence alignments of Cas nucleases according to embodiments of the present teachings. FIGS. 34A-O disclose SEQ ID NOS 611-631, respectively, in order of appearance;

FIG. 34I shows section 9 of sequence alignments of Cas nucleases according to embodiments of the present teachings. FIGS. 34A-O disclose SEQ ID NOS 611-631, respectively, in order of appearance;

FIG. 34J shows section 10 of sequence alignments of Cas nucleases according to embodiments of the present teachings. FIGS. 34A-O disclose SEQ ID NOS 611-631, respectively, in order of appearance;

FIG. 34K shows section 11 of sequence alignments of Cas nucleases according to embodiments of the present teachings. FIGS. 34A-O disclose SEQ ID NOS 611-631, respectively, in order of appearance;

FIG. 34L shows section 12 of sequence alignments of Cas nucleases according to embodiments of the present teachings. FIGS. 34A-O disclose SEQ ID NOS 611-631, respectively, in order of appearance;

FIG. 34M shows section 13 of sequence alignments of Cas nucleases according to embodiments of the present teachings. FIGS. 34A-O disclose SEQ ID NOS 611-631, respectively, in order of appearance;

FIG. 34N shows section 14 of sequence alignments of Cas nucleases according to embodiments of the present teachings. FIGS. 34A-O disclose SEQ ID NOS 611-631, respectively, in order of appearance; and

FIG. 34O shows section 15 of sequence alignments of Cas nucleases according to embodiments of the present teachings. FIGS. 34A-O disclose SEQ ID NOS 611-631, respectively, in order of appearance.

### DETAILED DESCRIPTION

It will be appreciated that for clarity, the following discussion will describe various aspects of embodiments of the applicant's teachings. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2nd edition (1989) (Sambrook, Fritsch, and Maniatis); Molecular Cloning: A Laboratory Manual, 4th edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2nd edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N. Y. 1992); and Marten H. Hofker and Jan van Deursen, Transgenic Mouse Methods and Protocols, 2nd edition (2011).

As used herein, the singular forms “a”, “an,” and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, +/−0.5% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

### Overview

The embodiments disclosed herein provide (non-naturally occurring or engineered) constructs, compositions, systems, and methods for site-directed RNA editing of RNA molecules. For example, the present invention provides (non-naturally occurring or engineered) methods for inhibiting intra and inter-cellular signaling pathways by modification of post-translational modification sites on select target RNA molecules. In certain example embodiments, the present invention provides (non-naturally occurring or engineered) methods for inhibiting intracellular phosphorylation of serine, threonine and tyrosine residues by editing the genetic codon of these amino acids by means of site-directed RNA editing or RNA molecules. Embodiments disclosed herein further provide methods of inhibiting pathological activation of cell signaling mediated by post-translational modifications, such as phosphorylation, which are involved in many diseases, including cancer, immunodeficiency, infectious diseases, inflammatory disorders and neurodegenerative disorders. The RNA-editing modification may be aimed at a single post-translational modification site of a single gene, but can also be multiplexed by targeting multiple sites on the same or different genes to increase efficacy. These approaches may be further combined with other treatments such as radiation, chemotherapy, targeted therapy based on antibodies or small molecules, and immunotherapy, which may have a synergistic effect.

The embodiments disclosed herein provide (non-naturally occurring or engineered) systems, constructs, and methods for targeted base editing. In general, the systems disclosed herein comprise a targeting component and a base editing component. The targeting component may function to specifically target the base editing component to a target nucleotide sequence in which one or more nucleotides are to be edited. The base editing component may then catalyze a chemical reaction to convert a first nucleotide in the target sequence to a second nucleotide. For example, the base editor may catalyze conversion of an adenine such that it is read as guanine by a cell's transcription or translation machinery, or vice versa. Likewise, the base editing component may catalyze conversion of cytidine to an uracil, or vice versa. In certain example embodiments, the base editor may be derived by starting with a known base editor, such as an adenine deaminase or cytidine deaminase, and modified using methods such as directed evolution to derive new functionalities. Directed evolution techniques are known in the art and may include those described in WO 2015/184016 “High-Throughput Assembly of Genetic Permutations.”

### Compositions and Systems

The present disclosure provides (non-naturally occurring or engineered) systems for editing a nucleic acid such as a gene or a product thereof (e.g., the encoded RNA or protein). In some embodiments, the systems may be an engineered, non-naturally occurring system suitable for modifying post-translational modification sites on proteins encoded by a target nucleic acid sequence. In certain cases, the target nucleic acid sequence is RNA, e.g., mRNA or a fragment thereof. In certain cases, the target nucleic acid sequence is DNA, e.g., a gene or a fragment thereof. In general, the system may comprise one or more of a catalytic inactive (dead) Cas protein (e.g., dead Cas7-11), a nucleotide deaminase protein or catalytic domain thereof, and a guide molecule. In certain examples, the nucleotide deaminase protein may be an adenosine deaminase. In certain examples, the nucleotide deaminase protein may be a cytidine deaminase. The guide sequence may be designed to have a degree of complementarity with a target sequence at one or more codons comprising an adenine or cytidine and that is post-translationally modified.

### CRISPR-Cas

Some embodiments disclosed herein are directed to CRISPR-Cas (clustered regularly interspaced short palindromic repeats associated proteins) systems. In the conflict between bacterial hosts and their associated viruses, CRISPR-Cas systems provide an adaptive defense mechanism that utilizes programmed immune memory. CRISPR-Cas systems provide their defense through three stages: adaptation, the integration of short nucleic acid sequences into the CRISPR array that serves as memory of past infections; expression, the transcription of the CRISPR array into a pre-crRNA (CRISPR RNA) transcript and processing of the pre-crRNA into functional crRNA species targeting foreign nucleic acids; and interference, the programming of CRISPR effectors by crRNA to cleave nucleic acid of foreign threats. Across all CRISPR-Cas systems, these fundamental stages display enormous variation, including the identity of the target nucleic acid (either RNA, DNA, or both) and the diverse domains and proteins involved in the effector ribonucleoprotein complex of the system.

CRISPR-Cas systems can be broadly split into two classes based on the architecture of the effector modules involved in pre-crRNA processing and interference. Class 1 systems have multi-subunit effector complexes composed of many proteins, whereas Class 2 systems rely on single-effector proteins with multi-domain capabilities for crRNA binding and interference; Class 2 effectors often provide pre-crRNA processing activity as well. Class 1 systems contain 3 types (type I, III, and IV) and 33 subtypes, including the RNA and DNA targeting type III-systems. Class 2 CRISPR families encompass 3 types (type II, V, and VI) and 17 subtypes of systems, including the RNA-guided DNases Cas9 and Cas12 and the RNA-guided RNase Cas13. Continual sequencing of novel bacterial genomes and metagenomes uncovers new diversity of CRISPR-Cas systems and their evolutionary relationships, necessitating experimental work that reveals the function of these systems and develops them into new tools.

Among the currently known CRISPR-Cas systems, only the type III and type VI systems have been demonstrated to bind and target RNA, and these two systems have substantially different properties, the most distinguishing being their membership in Class 1 and Class 2, respectively. Characterized subtypes of type III, which span type III-A, B, and C systems, target both RNA and DNA species through an effector complex containing multiple Cas7 (Csm3/5 or Cmr1/4/6) RNA nuclease units in association with a single Cas10 (Csm1 or Cmr2) DNA nuclease. The RNA nuclease activity of Cas7 is mediated through acidic residues in the repeat-associated mysterious proteins (RAMP) domains, which cut at stereotyped intervals in the guide:target duplex. Type III systems also have a target restriction, and cannot efficiently target protospacers in vivo if there is extended homology between the 5′ “tag” of the crRNA and the “anti-tag” 3′ of the protospacer in the target, although this binding does not block RNA cleavage in vitro. In type III systems, pre-crRNA processing is carried out by either host factors or the associated Cas6 family protein, which can physically complex with the effector machinery.

In contrast to type III systems, type VI systems contain a single CRISPR effector Cas13 that can only effect RNA interference, mediated through basic catalytic residues of dual HEPN domains. This interference requires a protospacer flanking sequence (PFS), although the influence of the PFS varies between orthologs and families. Importantly, the RNA cleavage activity of Cas13, once triggered by crRNA:target duplex formation, is indiscriminate, and activated Cas13 enzymes will cleave other RNA species in vitro, in bacterial hosts, and mammalian cells. This activity, termed the collateral effect, has been applied to CRISPR-based nucleic acid detection technologies. In addition to the RNA interference activity, the Cas13 family members contain pre-crRNA processing activity. Just as single-effector DNA targeting systems have given rise to numerous genome editing applications, Cas13 family members have been applied to a suite of RNA-targeting technologies in both bacterial and eukaryotic cells, including RNA knockdown, RNA editing, RNA tracking, epitranscriptome editing, translational upregulation, epi-transcriptomic reading and writing via N6-Methyladenosine, and isoform modulation.

The novel type III-E system was recently identified from genomes of 8 bacterial species and is characterized as a fusion of several Cas7 proteins and a putative Cas11 (Csm2)-like small subunit. The domain composition suggests the fusion of multiple type III effector module domains involved in crRNA binding into a single protein effector that is predicted to process pre-crRNA given its homology with Cas5 (Csm4) and conserved aspartates. The lack of other putative effector nucleases in these CRISPR loci raise the additional possibility that this fusion protein is capable of crRNA-directed RNA cleavage. If so, this system would blur the distinction of Class 1 and Class 2 systems, as it would have domains homologous to other Class 1 systems, but possess a single effector module characteristic of Class 2 systems. Beyond the single effector module present in all subtype III-E loci, a majority of type III-E family members contain a putative ancillary gene with a CHAT domain, which is a caspase family protease associated with programmed cell death (PCD), suggesting involvement of PCD-mediated antiviral strategies, as has been observed with type III and VI systems.

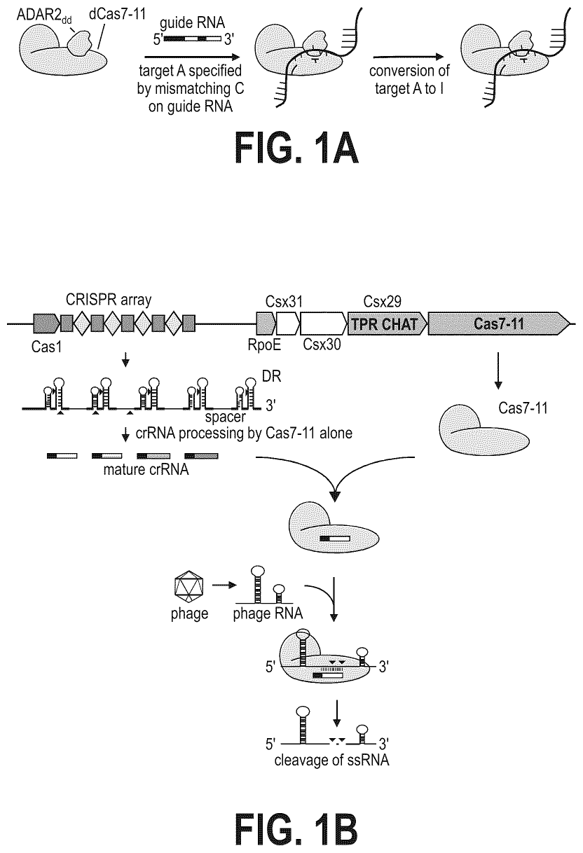

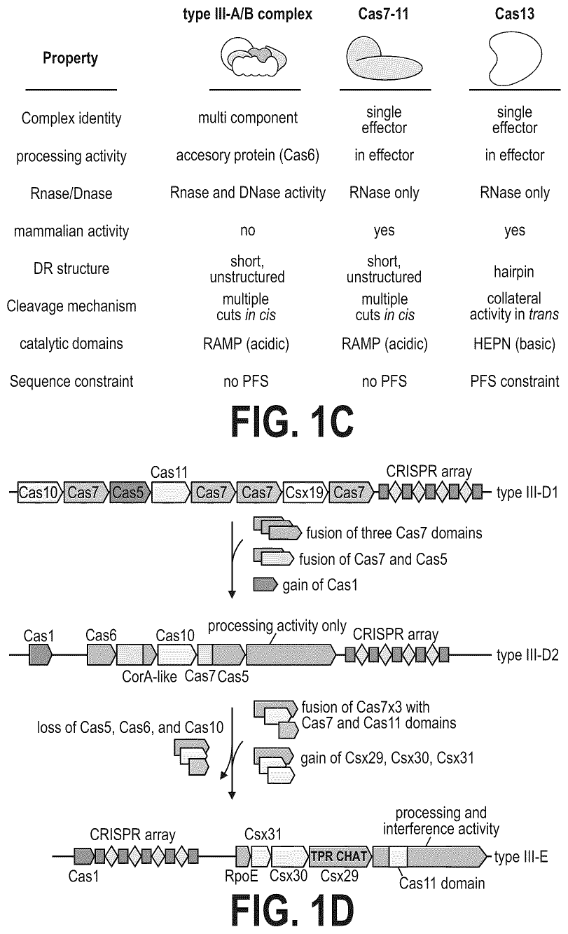

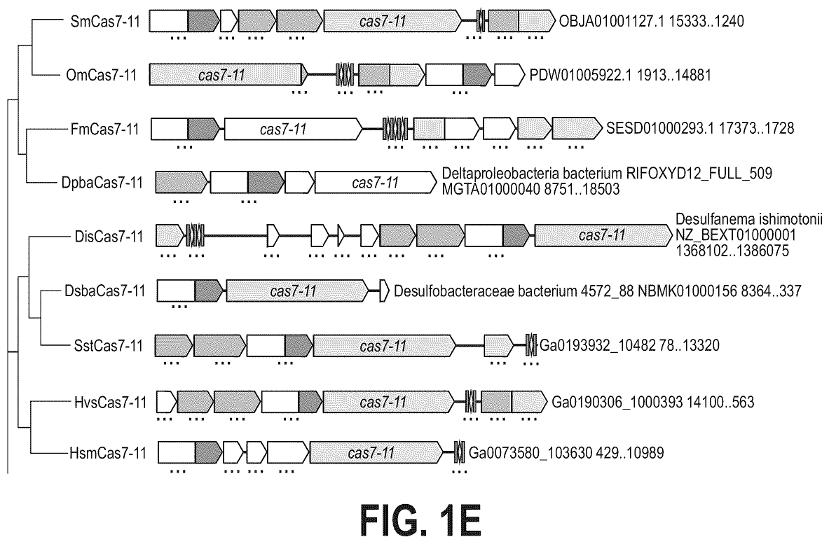

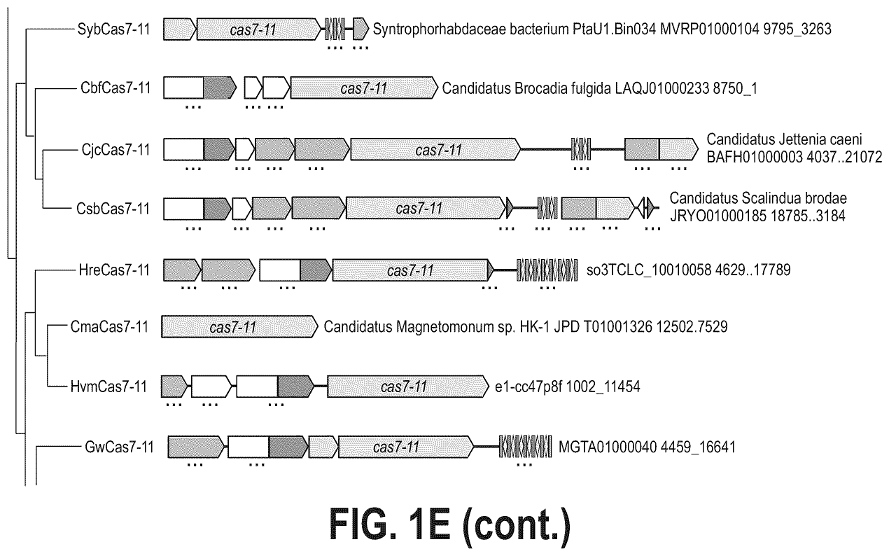

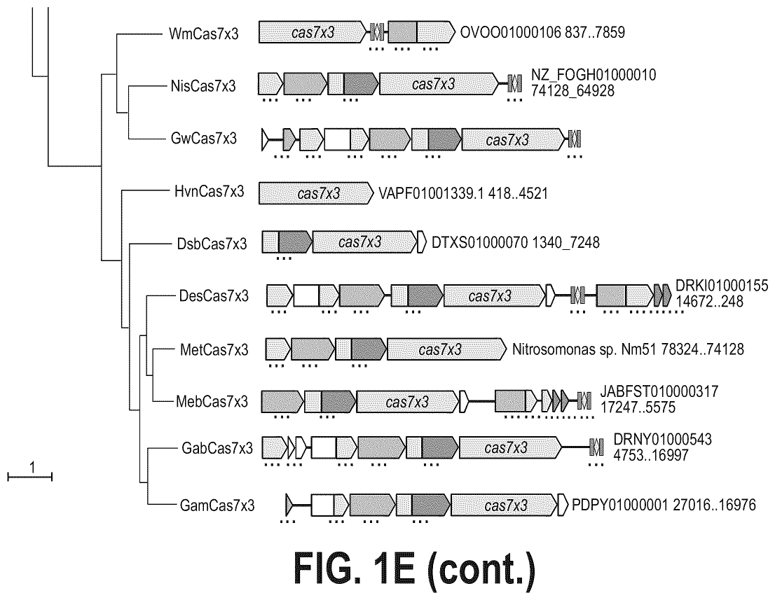

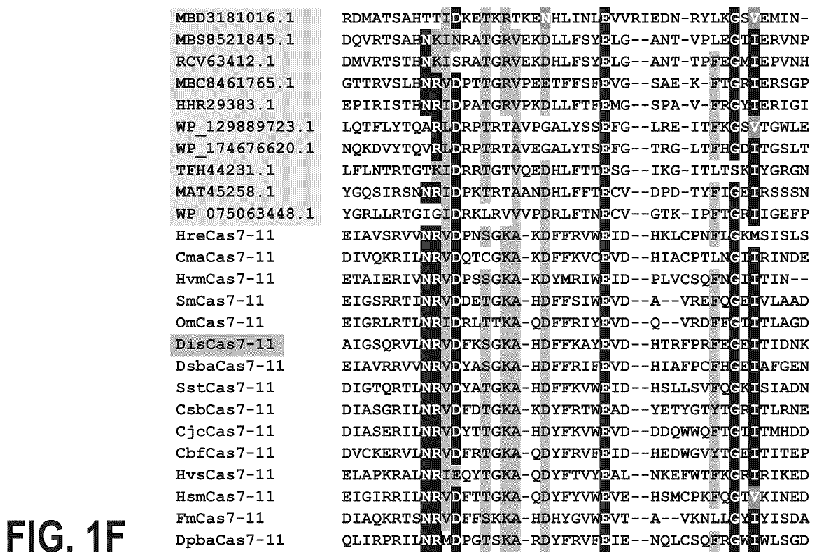

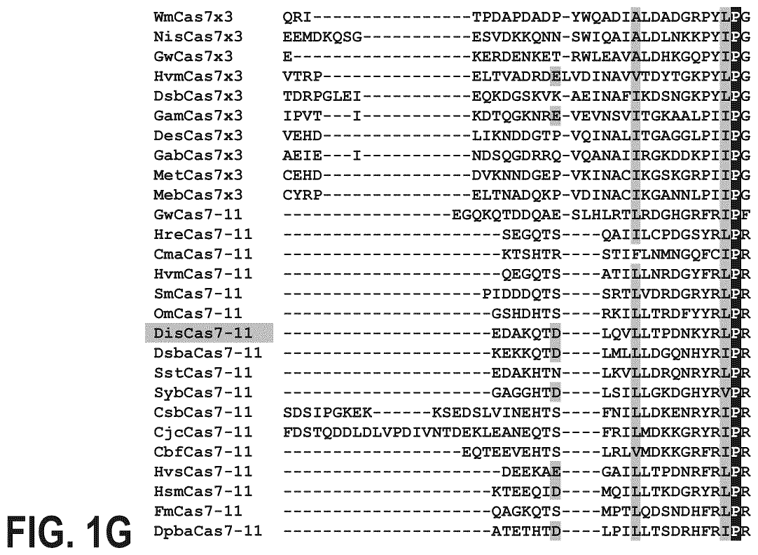

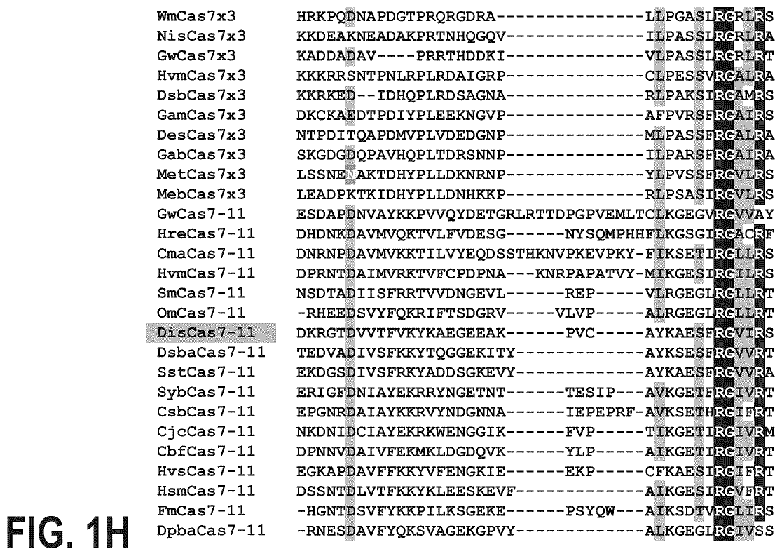

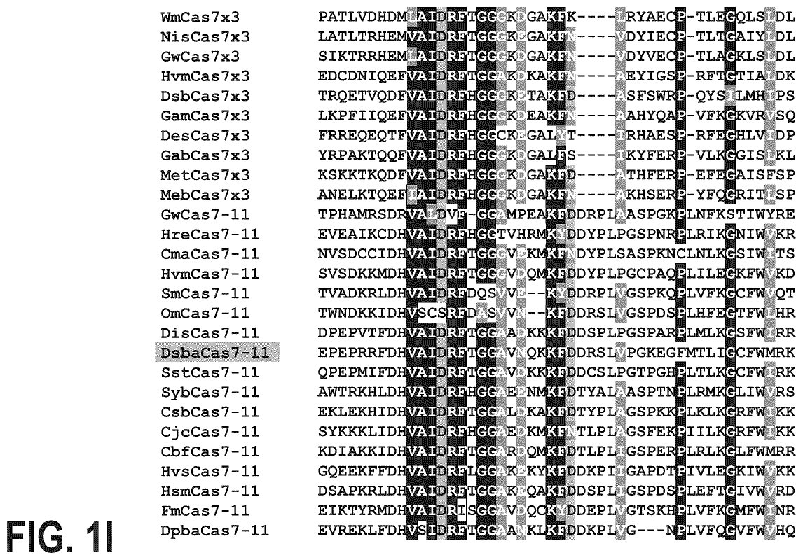

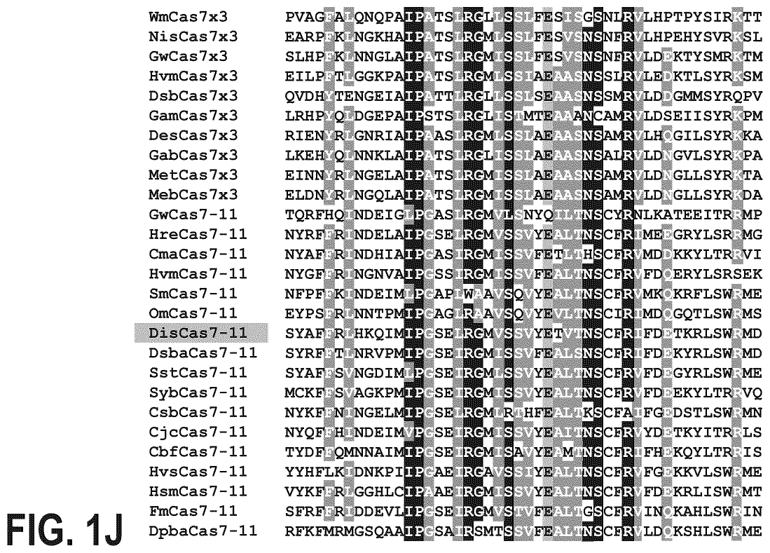

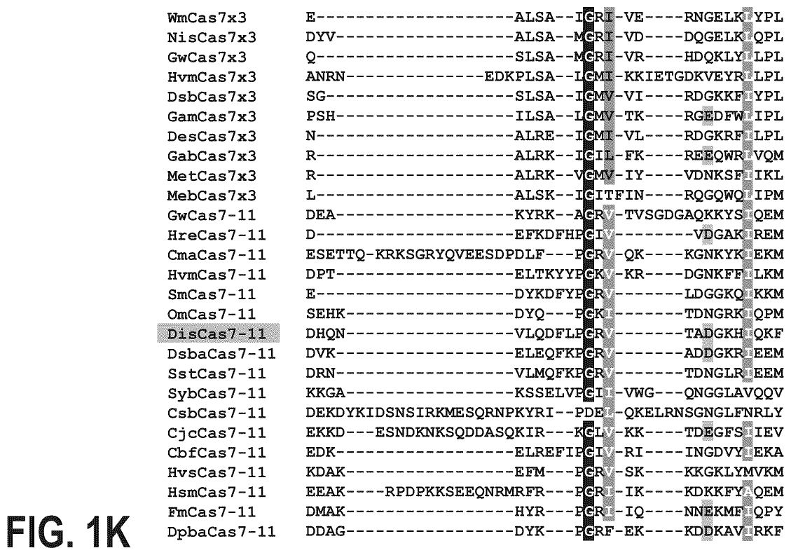

Type III-E system associated effector, interchangeably termed Cas15 and Cas7-11 herein, is a programmable RNase (FIGS. 1A-1D). This system can provide defense against RNA phage and be programmed to target exogenous mRNA species when expressed heterologously in bacteria. Orthologs of Cas7-11 are capable of both processing of pre-crRNA and crRNA-directed cleavage of RNA targets, and determine catalytic residues underlying programmed RNA cleavage. A direct evolutionary path of Cas7-11 can be traced from individual Cas7 and Cas11 effector proteins of subtype III-D1 variant, through an intermediate, a partially fused effector Cas7x3 of the subtype III-D2 variant, to the singe-effector architecture of subtype III-E that is so far unique among the Class 1 CRISPR-Cas systems (FIG. 1D). Cas7-11 most likely originated from two type III-D variants. Three Cas7 domains (domains 3, 4 and 5) are derived from subtype III-D2 that contains a the Cas7x3 effector protein along with Cas10 and another Cas7-like domain fused to a Cas5-like domain. The origin of the N-terminal Cas7 and putative Cas11 domain of Cas7-11 is most likely derived from a III-D1 variant, where both genes are stand-alone.

Cas7-11 differs substantially from Cas13, in terms of both domain organization and activity (FIG. 1C). Cas13 RNA cleavage is enacted by dual HEPN domains with basic catalytic residues, and this cleavage, once triggered, is indiscriminate. In contrast, Cas7-11 utilizes at least two of four Cas7-like domains with acidic catalytic residues to generate stereotyped cleavage at the target binding site in cis. Furthermore, Cas13 targeting is restricted by the requirement for a PFS, which Cas7-11 does not require, and the DR of Cas7-11-associated crRNA is substantially shorter. Because of these unique features, Cas7-11 may have distinct advantages for RNA targeting and transcriptome engineering biotechnology applications.

Regulation of interference by accessory proteins has been observed in both type III and type VI systems, and other proteins in the D. ishimotonii type III-E locus can regulate activity of DisCas7-11a. Notably, TPR-CHAT had a strong inhibitory effect on DisCas7-11a phage interference, raising the possibility that unrestricted DisCas7-11a activity could be detrimental for the host. Alternatively, as TPR-CHAT is a caspase family protease associated with programmed cell death (PCD), it is possible that TPR-CHAT is activated by DisCas7-11a and leads to host death, which could mimic death due to phage in these assays. TPR-CHAT caspase activity could be activated by DisCas7-11a and cause PCD through general proteolysis, analogous to PCD triggered by Cas13 collateral activity.

Similar to Class 2 CRISPR effectors such as Cas9, Cas12, and Cas13, Cas7-11 is highly active in mammalian cells, with substantial knockdown activity on both reporter and endogenous transcripts. Moreover, via inactivation of active sites through mutagenesis, the catalytically inactive dCas7-11 enzyme can be used to recruit ADAR2DD for efficient site-specific A-to-I editing on transcripts. These applications establish Cas7-11 as the basis for an RNA-targeting toolbox that has several benefits compared to Cas13, including the lack of sequence preferences and collateral activity, the latter of which has been shown to induce toxicity in certain cell types. A Cas7-11 toolbox may serve as the basis for multiple RNA technologies, including RNA knockdown, RNA editing, translation modulation, RNA recruitment, RNA tracking, splicing control, RNA stabilization, and potentially even diagnostics.

### AD-Functionalized CRISPR Systems

In some embodiments, the systems may be AD-functionalized CRISPR system. The term “AD-functionalized CRISPR system” as used here refers to a nucleic acid targeting and editing system comprising (a) a CRISPR-Cas protein, more particularly a Cas7-11 protein which is catalytically active or inactive; (b) a guide molecule which comprises a guide sequence; and (c) an adenosine deaminase (AD) protein or catalytic domain thereof; wherein the adenosine deaminase protein or catalytic domain thereof is covalently or non-covalently linked to the CRISPR-Cas protein or the guide molecule or is adapted to link thereto after delivery; wherein the guide sequence is substantially complementary to the target sequence but comprises a non-pairing C corresponding to the A being targeted for deamination, resulting in an A-C mismatch in an RNA duplex formed by the guide sequence and the target sequence. In some embodiments, the CRISPR-Cas protein and/or the adenosine deaminase comprise one or more heterologous nuclear export signal(s) (NES(s)) or nuclear localization signal(s) (NLS(s)). For application in eukaryotic cells, the CRISPR-Cas protein and/or the adenosine deaminase can be NES-tagged or NLS-tagged.

One skilled in the art would appreciate that the components (a), (b) and (c) can be delivered to the cell as a ribonucleoprotein complex. The ribonucleoprotein complex can be delivered via one or more lipid nanoparticles. One skilled in the art would appreciate that the components (a), (b) and (c) can be delivered to the cell as one or more RNA molecules, such as one or more guide RNAs and one or more mRNA molecules encoding the CRISPR-Cas protein, the adenosine deaminase protein, and optionally the adaptor protein. The RNA molecules can be delivered via one or more lipid nanoparticles. One skilled in the art would appreciate that the components (a), (b) and (c) can be delivered to the cell as one or more DNA molecules. The one or more DNA molecules can be comprised within one or more vectors such as viral vectors (e.g., AAV). The one or more DNA molecules can comprise one or more regulatory elements operably configured to express the CRISPR-Cas protein, the guide molecule, and the adenosine deaminase protein or catalytic domain thereof, optionally wherein the one or more regulatory elements comprise inducible promoters.

In some embodiments, the CRISPR-Cas protein is a dead Cas7-11. In some embodiments, the dead Cas7-11 comprises one or more mutations in the Cas7-like domains, including D429A and D654A as well as many other mutations (see table 1).

In some embodiments, the guide molecule is capable of hybridizing with a target sequence comprising the Adenine to be deaminated within an RNA sequence to form an RNA duplex which comprises a non-pairing Cytosine opposite to said Adenine. Upon RNA duplex formation, the guide molecule forms a complex with the Cas7-11 protein and directs the complex to bind the RNA polynucleotide at the target RNA sequence of interest. Details on the aspect of the guide of the AD-functionalized CRISPR-Cas system are provided herein below.

In at least a first design, the AD-functionalized CRISPR system comprises: (a) an adenosine deaminase fused or linked to a CRISPR-Cas protein, wherein the CRISPR-Cas protein is catalytically inactive; and (b) a guide molecule comprising a guide sequence designed to introduce an A-C mismatch in an RNA duplex formed between the guide sequence and the target sequence. In some embodiments, the CRISPR-Cas protein and/or the adenosine deaminase can be NLS-tagged on either the N- or C-terminus or both.

In at least a second design, the AD-functionalized CRISPR system comprises: (a) a CRISPR-Cas protein that is catalytically inactive; (b) a guide molecule comprising a guide sequence designed to introduce an A-C mismatch in an RNA duplex formed between the guide sequence and the target sequence, and an aptamer sequence (e.g., MS2 RNA motif or PP7 RNA motif) capable of binding to an adaptor protein (e.g., MS2 coating protein or PP7 coat protein); and (c) an adenosine deaminase fused or linked to an adaptor protein, wherein the binding of the aptamer and the adaptor protein recruits the adenosine deaminase to the RNA duplex formed between the guide sequence and the target sequence for targeted deamination at the A of the A-C mismatch. In some embodiments, the adaptor protein and/or the adenosine deaminase can be NLS-tagged, on either the N- or C-terminus or both. The CRISPR-Cas protein can also be NLS-tagged. The CRISPR-Cas protein can also be NLS-tagged.

The use of different aptamers and corresponding adaptor proteins also allows orthogonal gene editing to be implemented. In one example in which adenosine deaminase are used in combination with cytidine deaminase for orthogonal gene editing/deamination, sgRNA targeting different loci are modified with distinct RNA loops in order to recruit MS2-adenosine deaminase and PP7-cytidine deaminase (or PP7-adenosine deaminase and MS2-cytidine deaminase), respectively, resulting in orthogonal deamination of A or C at the target loci of interested, respectively. PP7 is the RNA-binding coat protein of the bacteriophage Pseudomonas. Like MS2, it binds a specific RNA sequence and secondary structure. The PP7 RNA-recognition motif is distinct from that of MS2. Consequently, PP7 and MS2 can be multiplexed to mediate distinct effects at different genomic loci simultaneously. For example, an sgRNA targeting locus A can be modified with MS2 loops, recruiting MS2-adenosine deaminase, while another sgRNA targeting locus B can be modified with PP7 loops, recruiting PP7-cytidine deaminase. In the same cell, orthogonal, locus-specific modifications are thus realized. This principle can be extended to incorporate other orthogonal RNA-binding proteins.

In at least a third design, the AD-functionalized CRISPR system comprises: (a) an adenosine deaminase inserted into an internal loop or unstructured region of a CRISPR-Cas protein, wherein the CRISPR-Cas protein is catalytically inactive or a nickase; and (b) a guide molecule comprising a guide sequence designed to introduce an A-C mismatch in an RNA duplex formed between the guide sequence and the target sequence.

The AD-functionalized CRISPR system described herein can be used to target a specific Adenine within an RNA polynucleotide sequence for deamination. For example, the guide molecule can form a complex with the CRISPR-Cas protein and directs the complex to bind a target RNA sequence in the RNA polynucleotide of interest. Because the guide sequence is designed to have a non-pairing C, the RNA duplex formed between the guide sequence and the target sequence comprises an A-C mismatch, which directs the adenosine deaminase to contact and deaminate the A opposite to the non-pairing C, converting it to an Inosine (I). Since Inosine (I) base pairs with C and functions like G in cellular processes, the targeted deamination of A described herein are useful for correction of undesirable G-A and C-T mutations, as well as for obtaining desirable A-G and T-C mutations.

In some embodiments, the AD-functionalized CRISPR system is used for targeted deamination in an RNA polynucleotide molecule in vitro. In some embodiments, the AD-functionalized CRISPR system is used for targeted deamination in a DNA molecule and/or RNA molecule within a cell. The cell can be an eukaryotic cell such as a bacteria or cyanobacteria. The cell can be a eukaryotic cell, such as an animal cell, a mammalian cell, a human, or a plant cell.

The invention also relates to a (non-naturally occurring or engineered) method for treating or preventing a disease by the targeted deamination using the AD-functionalized CRISPR system, wherein the deamination of the A, which remedies a disease caused by transcripts containing a pathogenic G→A or C→T point mutation. Examples of disease that can be treated or prevented with the present invention include cancer, Meier-Gorlin syndrome, Seckel syndrome 4, Joubert syndrome 5, Leber congenital amaurosis 10; Charcot-Marie-Tooth disease, type 2; Charcot-Marie-Tooth disease, type 2; Usher syndrome, type 2C; Spinocerebellar ataxia 28; Spinocerebellar ataxia 28; Spinocerebellar ataxia 28; Long QT syndrome 2; Sjogren-Larsson syndrome; Hereditary fructosuria; Hereditary fructosuria; Neuroblastoma; Neuroblastoma; Kallmann syndrome 1; Kallmann syndrome 1; Kallmann syndrome 1; Metachromatic leukodystrophy.

AD-functionalized CRISPR system for RNA editing can be used for translation upregulation or downregulation, improving RNA stability and diagnostics. For example, for application in diagnostics, TPR-Chat is an accessory protein that interacts with Cas7-11 interference. Cas7-11 can activate TPR-Chat caspase activity which can then activate a reporter. While this can be used for inducing cell death based on RNA detection (e.g., in cancer cells), it also can be useful for general RNA diagnostics (i.e. molecular diagnostics for bacteria, viruses, and derivatives thereof) in samples. Furthermore, Cas7-11 can re-constitute a split protein like GFP on a specific transcript.

AD-functionalized CRISPR system for RNA editing can be used to treat or prevent premature termination diseases. Pre-termination diseases are characterized by mutations in early stop codons, either through single nucleotide polymorphisms that introduce termination, indels that change the translational frame of the protein and generate new stop codons, or alternative splicing that preferentially introduces exons that have early termination. By removing stop codons generated in these ways via A to I editing, RNA editing with ADAR could rescue diseases involving premature termination. In cases where SNPs are not G to A, but generate nonsense mutations, clinical benefit could be derived from changing nonsense mutations into missense mutations.

AD-functionalized CRISPR system for RNA editing can be used to change fertility mutations without germline editing. One advantage of RNA editing over DNA editing is in cases of SNPs affecting fertility, where correction with genome editing would necessarily result in germline editing, with potential ethical or safety implications. RNA editing could correct these mutations without permanent effects on the genome, thereby circumventing these issues.

AD-functionalized CRISPR system for RNA editing can be used for splicing alteration. Pre-mRNA requires specific splice donor and acceptor sequences in order to undergo processing by the spliceosome. Splice acceptor sites contain an invariant AG sequence that is necessary for acceptance of the attack by the splice donor sequence and intron removal. By targeting Cas7-11-ADAR fusions to pre-mRNA and editing AG splice acceptor sites to IG, it can be possible to inactivate the splice acceptor site, resulting in skipping of the downstream exon. This approach to splicing alteration has advantages over the current method of exon skipping with chemically modified anti-sense oligos. Cas7-11-ADAR can be genetically encoded, allowing for long-term exon skipping. Additionally, Cas7-11-ADAR creates a mutation to promote skipping, which can be more robust than masking of the splice donor/acceptor site by a double stranded RNA, as is done with anti-sense oligos.

AD-functionalized CRISPR system for RNA editing can be used to alter neoantigens. Neoantigens in cancer are novel antigens that are expressed in tumor cells due to mutations that arise because of defective mismatch repair. Engineering T cells against neoantigens is advantageous because the T cells will have no off-target activity and thus toxicity since the antigens are only expressed in the tumor cells. With RNA base editors, the Cas7-11-ADAR fusions can be targeted to cancer cells to introduce mutations in transcripts that would introduce amino acid changes and new antigens that can be targeted using chimeric antigen receptor T cells. This approach is better than DNA base editors because it is transient and thus the risk of editing non-tumor cells permanently due to off-target delivery is minimal.

AD-functionalized CRISPR system for RNA editing can be used to change microRNA targets for tumor suppressors. ADAR naturally edits mRNA to generate or remove microRNA targets, thereby modulating expression. Programmable RNA editing can be used to up- or down-regulate microRNA targets via altering of targeting regions. Additionally, microRNAs themselves are natural substrates for ADAR, and programmable RNA editing of micoRNAs can reduce or enhance the function on their corresponding targets.

AD-functionalized CRISPR system for RNA editing can be used to make multiple edits along a region. The Cas7-11-ADAR fusions can be precisely targeted to edit specific adenosines by introducing a mismatch in the guide region across from the desired adenosine target and creating a bubble that is favorable for A-to-I editing. By introducing multiple of these mismatches across different adenosine sites in the guide/target duplex, it can be possible to introduce multiple mutations at once.

AD-functionalized CRISPR system for RNA editing can be used for the reversal of TAA (double A to G) for PTC. Many diseases that involve pretermination codon changes involve a TAA stop codon, which would require A-to-I changes to correct rather than the TAG or TGA stop codons which only need one A-to-I edit. Two approaches can be used to reverse the TAA stop codon. (1) As described in the previous section, two mismatches can be introduced in the guide against the two adenosines in the TAA codon. (2) A two-guide array can be used to convert each of the adenosines to inosine sequentially. The first guide in the array can contain a mutation against the first adenosine and the second guide can then have complementarity to this change and have a mismatch against the second adenosine in the stop codon.

AD-functionalized CRISPR system for RNA editing can be used to treat or prevent cancer (GOF, LOF mutation reversal). Many oncogenic changes in cancer involve G to A mutations that introduce gain of function or loss of function phenotypes to the mutated proteins. The RNA base editors are well positioned to correct these changes and reduce oncogenesis.

RNA editing with ADAR can be used for the design of new base preferences. Current ADAR1/2 proteins have been found to have surrounding base preferences for catalytic activity, which may pose constraints for certain applications. Rational mutagenesis or directed evolution of ADAR variants with altered or relaxed base preferences can increase the versatility of programmable RNA editing.

AD-functionalized CRISPR system for RNA editing can comprise ADAR mutants with increased activity in human cells. Although ADAR mutants with altered activity in vitro or in yeast have been previously reported, screening or rational design of mutants with increased activity in the context of human cells can improve the efficiency or specificity of ADAR-based programmable RNA editing constructs.

AD-functionalized CRISPR system for RNA editing can be used in biological applications of inosine generation. The RNA editing with ADAR generates inosine, which, when occurring multiple times in a transcript, can interact with endogenous biological pathways to increase inflammation in cells and tissues. Generation of multiple inosine bases can increase inflammation, especially in cells where inflammation can lead to clearance. Additional inosine generation could also be used to destabilize transcripts.

AD-functionalized CRISPR system for RNA editing can be used in removing upstream start codons to promote protein expression of downstream ORF (ATG mutation). Anti-sense oligos have been used for blocking upstream start codon sites to promote protein expression at downstream start codons. This allows the boosting of endogenous protein levels for therapeutic purposes. Cas7-11-ADAR fusions could accomplish a similar effect by converting ATG sites to ITG (GTG) sites and thus remove upstream codons in endogenous transcripts and thus boost protein translation. So far, most therapeutic applications discussed have been for correcting G to A mutations or removing pre-termination sites. This would be an application that allows for boosting gene expression. A good example is boosting fetal hemoglobin levels in sickle cell disease and thalassemias.

AD-functionalized CRISPR system for RNA editing can comprise the mutagenesis of ADAR for C to U or any transition. It is possible through rational mutagenesis or directed evolution that the ADARs listed in the ortholog section could be made into C to U editors or editors of any base transition.

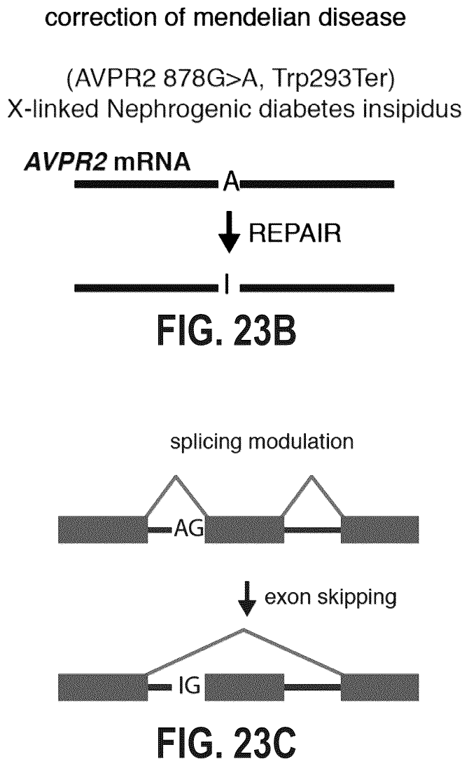

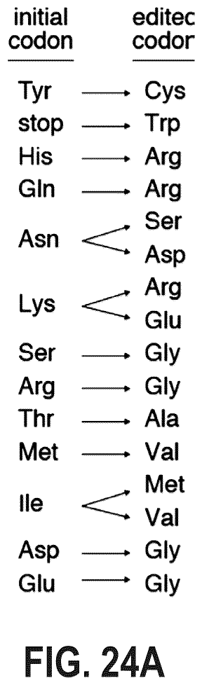

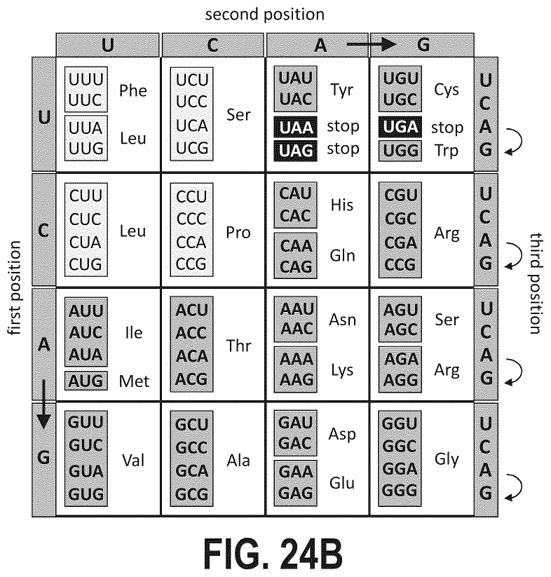

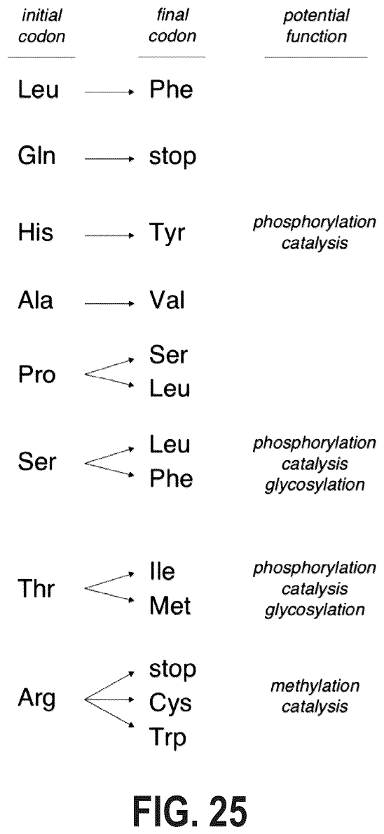

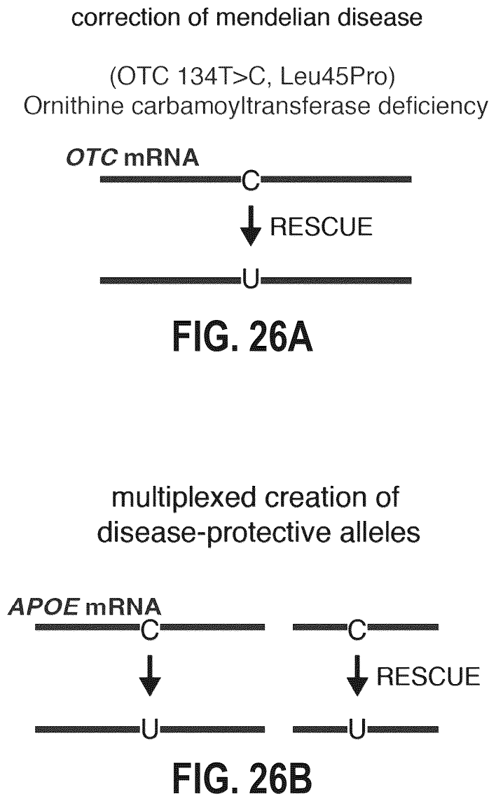

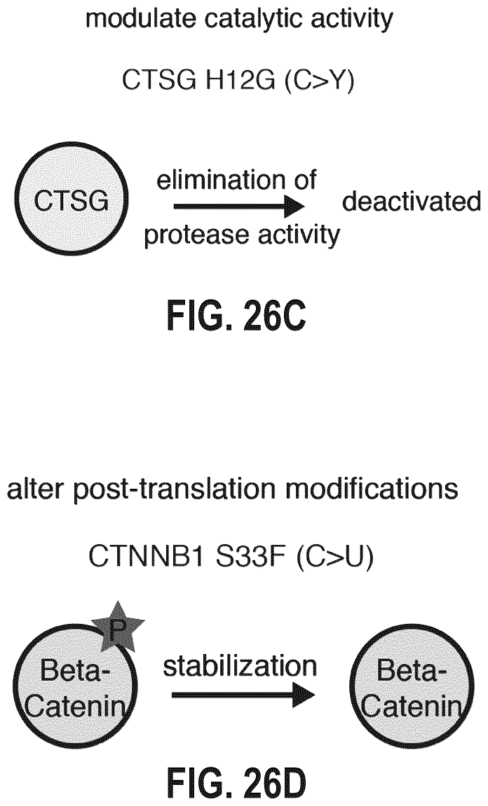

FIGS. 23-26 illustrates codon changes and their effects when RNA editors are used. FIG. 23A shows that modulation can be used for modifying protein function through amino acid changes that affect enzyme domains, such as kinases. FIG. 23B shows that modulation can be used for correction of Mendelian disease mutations. FIG. 23C shows the splicing modulation of transcripts by modifying the splice acceptor site. FIGS. 24A-B show potential codon transitions enabled by A>I editing. FIG. 25 shows show potential codon transitions and function enabled by C>U editing. Adapted and modified based on J. D. Watson, Molecular biology of the gene. (Pearson, Boston, ed. Seventh edition, 2014), pp. xxxiv, 872 pages (38). FIG. 66C). FIG. 26A shows that C-U modulation can be used for correction of Mendelian disease mutations. FIG. 26B shows the multiplexed creation of disease-protective alleles that requires C to U RNA editing. FIG. 26C shows the modulation of catalytic activity that requires C to U RNA editing. FIG. 26D shows alter post-translational modifications that requires C to U RNA editing.

In particular embodiments, the compositions described herein can be used in therapy. This implies that the methods can be performed in vivo, ex vivo or in vitro. In particular embodiments, the methods can be not methods of treatment of the animal or human body or a method for modifying the germ line genetic identity of a human cell. In particular embodiments, when carrying out the method, the target RNA can be not comprised within a human or animal cell. In particular embodiments, when the target is a human or animal target, the method can be carried out ex vivo or in vitro.

### CRISPR-Cas Proteins and Guides

In some embodiments, the system comprises one or more components of a CRISPR-Cas system. For example, the system may comprise a Cas protein, a guide molecule, or a combination thereof.

In the methods and systems of the present invention use is made of a CRISPR-Cas protein and corresponding guide molecule. More particularly, the CRISPR-Cas protein is a class 2 CRISPR-Cas protein. In certain embodiments, said CRISPR-Cas protein is a Cas7-11. The Cas7-11 may be Cas7-11a, Cas7-11b, Cas7-11c, or Cas7-11d. The CRISPR-Cas system does not require the generation of customized proteins to target specific sequences but rather a single Cas protein can be programmed by guide molecule to recognize a specific nucleic acid target, in other words the Cas enzyme protein can be recruited to a specific nucleic acid target locus of interest using said guide molecule.

### CRISPR-Cas Proteins

In some embodiments, the systems may comprise a CRISPR-Cas protein. In certain examples, the CRISPR-Cas protein may be a catalytically inactive (dead) Cas protein. The catalytically inactive (dead) Cas protein may have impaired (e.g., reduced or no) nuclease activity. In some cases, the dead Cas protein may have nickase activity. In some cases, the dead Cas protein may be dead Cas 15 protein. For example, the dead Cas 15 may be dead Cas7-11a, dead Cas7-11b, dead Cas7-11c, or dead Cas7-11d. In some embodiments, the system may comprise a nucleotide sequence encoding the dead Cas protein.

In its unmodified form, a CRISPR-Cas protein is a catalytically active protein. This implies that upon formation of a nucleic acid-targeting complex (comprising a guide RNA hybridized to a target sequence) one or both DNA strands in or near (e.g., within 1, 2, 3, 4, 5, 6, 7, 8, 9, 10, 20, 50, or more base pairs from) the target sequence is modified (e.g., cleaved). As used herein the term “sequence(s) associated with a target locus of interest” refers to sequences near the vicinity of the target sequence (e.g., within 1, 2, 3, 4, 5, 6, 7, 8, 9, 10, 20, 50, or more base pairs from the target sequence, wherein the target sequence is comprised within a target locus of interest). The unmodified catalytically active Cas7-11 protein generates a staggered cut, whereby the cut sites are typically within the target sequence. More particularly, the staggered cut is typically 13-23 nucleotides distal to the PAM. In particular embodiments, the cut on the non-target strand is 17 nucleotides downstream of the PAM (i.e., between nucleotide 17 and 18 downstream of the PAM), while the cut on the target strand (i.e. strand hybridizing with the guide sequence) occurs a further 4 nucleotides further from the sequence complementary to the PAM (this is 21 nucleotides upstream of the complement of the PAM on the 3′ strand or between nucleotide 21 and 22 upstream of the complement of the PAM).

In the methods according to the present invention, the CRISPR-Cas protein is preferably mutated with respect to a corresponding wild-type enzyme such that the mutated CRISPR-Cas protein lacks the ability to cleave one or both DNA strands of a target locus containing a target sequence. In particular embodiments, one or more catalytic domains of the Cas7-11 protein are mutated to produce a mutated Cas protein which cleaves only one DNA strand of a target sequence.

In particular embodiments, the CRISPR-Cas protein may be mutated with respect to a corresponding wild-type enzyme such that the mutated CRISPR-Cas protein lacks substantially all DNA cleavage activity. In some embodiments, a CRISPR-Cas protein may be considered to substantially lack all DNA and/or RNA cleavage activity when the cleavage activity of the mutated enzyme is about no more than 25%, 10%, 5%, 1%, 0.1%, 0.01%, or less of the nucleic acid cleavage activity of the non-mutated form of the enzyme; an example can be when the nucleic acid cleavage activity of the mutated form is nil or negligible as compared with the non-mutated form.

In certain embodiments of the methods provided herein the CRISPR-Cas protein is a mutated CRISPR-Cas protein which cleaves only one DNA strand, i.e., a nickase. More particularly, in the context of the present invention, the nickase ensures cleavage within the non-target sequence, i.e., the sequence which is on the opposite DNA strand of the target sequence and which is 3′ of the PAM sequence.

In some embodiments, a CRISPR-Cas protein is considered to substantially lack all DNA cleavage activity when the DNA cleavage activity of the mutated enzyme is about no more than 25%, 10%, 5%, 1%, 0.1%, 0.01%, or less of the DNA cleavage activity of the non-mutated form of the enzyme; an example can be when the DNA cleavage activity of the mutated form is nil or negligible as compared with the non-mutated form. In these embodiments, the CRISPR-Cas protein is used as a generic DNA binding protein. The mutations may be artificially introduced mutations or gain- or loss-of-function mutations.

In addition to the mutations described above, the CRISPR-Cas protein may be additionally modified. As used herein, the term “modified” with regard to a CRISPR-Cas protein generally refers to a CRISPR-Cas protein having one or more modifications or mutations (including point mutations, truncations, insertions, deletions, chimeras, fusion proteins, etc.) compared to the wild type Cas protein from which it is derived. By derived is meant that the derived enzyme is largely based, in the sense of having a high degree of sequence homology with, a wildtype enzyme, but that it has been mutated (modified) in some way as known in the art or as described herein. The modification can be fusions of effectors like fluorophore, proteins involved in translation modulation (e.g., eIF4E, eIF4A, and eIF4G) and proteins involved with epitranscriptomic modulation (e.g. pseudouridine synthase and m6a writer/readers), and splicing factors involved with changing splicing. Cas7-11 could also be used for sensing RNA for diagnostic purposes. In some embodiments, the C-terminus of the Cas7-11 effector can be truncated. For example, at least 20 amino acids, at least 40 amino acids, at least 50 amino acids, at least 60 amino acids, at least 80 amino acids, at least 100 amino acids, at least 120 amino acids, at least 140 amino acids, at least 150 amino acids, at least 160 amino acids, at least 180 amino acids, at least 200 amino acids, at least 220 amino acids, at least 240 amino acids, at least 250 amino acids, at least 260 amino acids, at least 300 amino acids, or at least 350 amino acids may be truncated at the C-terminus of the Cas 13 effector. For example, up to 120 amino acids, up to 140 amino acids, up to 160 amino acids, up to 180 amino acids, up to 200 amino acids, up to 250 amino acids, up to 300 amino acids, up to 350 amino acids, or up to 400 amino acids may be truncated at the C-terminus of the Cas 15 effector.

In some embodiments, the N-terminus of the Cas7-11 effector protein may be truncated. For example, at least 20 amino acids, at least 40 amino acids, at least 50 amino acids, at least 60 amino acids, at least 80 amino acids, at least 100 amino acids, at least 120 amino acids, at least 140 amino acids, at least 150 amino acids, at least 160 amino acids, at least 180 amino acids, at least 200 amino acids, at least 220 amino acids, at least 240 amino acids, at least 250 amino acids, at least 260 amino acids, at least 300 amino acids, or at least 350 amino acids may be truncated at the N-terminus of the Cas7-11 effector. For examples, up to 120 amino acids, up to 140 amino acids, up to 160 amino acids, up to 180 amino acids, up to 200 amino acids, up to 250 amino acids, up to 300 amino acids, up to 350 amino acids, up to 400 amino acids may be truncated at the N-terminus of the Cas7-11 effector.

In some embodiments, both the N- and the C-termini of the Cas7-11 effector protein may be truncated. For example, at least 20 amino acids may be truncated at the C-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas7-11 effector. For example, at least 40 amino acids may be truncated at the C-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas7-11 effector. For example, at least 60 amino acids may be truncated at the C-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas7-11 effector. For example, at least 80 amino acids may be truncated at the C-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas7-11 effector. For example, at least 100 amino acids may be truncated at the C-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas7-11 effector. For example, at least 120 amino acids may be truncated at the C-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas7-11 effector. For example, at least 140 amino acids may be truncated at the C-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas7-11 effector. For example, at least 160 amino acids may be truncated at the C-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas7-11 effector. For example, at least 180 amino acids may be truncated at the C-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas7-11 effector. For example, at least 200 amino acids may be truncated at the C-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas7-11 effector. For example, at least 220 amino acids may be truncated at the C-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas7-11 effector. For example, at least 240 amino acids may be truncated at the C-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas7-11 effector. For example, at least 260 amino acids may be truncated at the C-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas7-11 effector. For example, at least 280 amino acids may be truncated at the C-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas7-11 effector. For example, at least 300 amino acids may be truncated at the C-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas7-11 effector. For example, at least 350 amino acids may be truncated at the C-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas7-11 effector. For example, at least 20 amino acids may be truncated at the N-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas7-11 effector. For example, at least 40 amino acids may be truncated at the N-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas7-11 effector. For example, at least 60 amino acids may be truncated at the N-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas7-11 effector. For example, at least 80 amino acids may be truncated at the N-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas7-11 effector. For example, at least 100 amino acids may be truncated at the N-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas7-11 effector. For example, at least 120 amino acids may be truncated at the N-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas7-11 effector. For example, at least 140 amino acids may be truncated at the N-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas7-11 effector. For example, at least 160 amino acids may be truncated at the N-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas7-11 effector. For example, at least 180 amino acids may be truncated at the N-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas7-11 effector. For example, at least 200 amino acids may be truncated at the N-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas7-11 effector. For example, at least 220 amino acids may be truncated at the N-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas7-11 effector. For example, at least 240 amino acids may be truncated at the N-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas7-11 effector. For example, at least 260 amino acids may be truncated at the N-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas7-11 effector. For example, at least 280 amino acids may be truncated at the N-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas7-11 effector. For example, at least 300 amino acids may be truncated at the N-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas7-11 effector. For example, at least 350 amino acids may be truncated at the N-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas7-11 effector.

The additional modifications of the CRISPR-Cas protein may or may not cause an altered functionality. By means of example, and in particular with reference to CRISPR-Cas protein, modifications which do not result in an altered functionality include for instance codon optimization for expression into a particular host, or providing the nuclease with a particular marker (e.g., for visualization). Modifications with may result in altered functionality may also include mutations, including point mutations, insertions, deletions, truncations (including split nucleases), etc. Fusion proteins may without limitation include for instance fusions with heterologous domains or functional domains (e.g., localization signals, catalytic domains, etc.). In certain embodiments, various modifications may be combined (e.g., a mutated nuclease which is catalytically inactive and which further is fused to a functional domain, such as for instance to induce DNA methylation or another nucleic acid modification, such as including without limitation a break (e.g. by a different nuclease (domain)), a mutation, a deletion, an insertion, a replacement, a ligation, a digestion, a break or a recombination). As used herein, “altered functionality” includes without limitation an altered specificity (e.g., altered target recognition, increased (e.g. “enhanced” Cas proteins) or decreased specificity, or altered PAM recognition), altered activity (e.g. increased or decreased catalytic activity, including catalytically inactive nucleases or nickases), and/or altered stability (e.g. fusions with destabilization domains). Suitable heterologous domains include without limitation a nuclease, a ligase, a repair protein, a methyltransferase, (viral) integrase, a recombinase, a transposase, an argonaute, a cytidine deaminase, a retron, a group II intron, a phosphatase, a phosphorylase, a sulpfurylase, a kinase, a polymerase, an exonuclease, etc. Examples of all these modifications are known in the art. It will be understood that a “modified” nuclease as referred to herein, and in particular a “modified” Cas or “modified” CRISPR-Cas system or complex preferably still has the capacity to interact with or bind to the polynucleic acid (e.g., in complex with the guide molecule). Such modified Cas protein can be combined with the deaminase protein or active domain thereof as described herein.

In certain embodiments, CRISPR-Cas protein may comprise one or more modifications resulting in enhanced activity and/or specificity, such as including mutating residues that stabilize the targeted or non-targeted strand (e.g., eCas9; “Rationally engineered Cas9 nucleases with improved specificity”, Slaymaker et al. (2016), Science, 351(6268):84-88, incorporated herewith in its entirety by reference). In certain embodiments, the altered or modified activity of the engineered CRISPR protein comprises increased targeting efficiency or decreased off-target binding. In certain embodiments, the altered activity of the engineered CRISPR protein comprises modified cleavage activity. In certain embodiments, the altered activity comprises increased cleavage activity as to the target polynucleotide loci. In certain embodiments, the altered activity comprises decreased cleavage activity as to the target polynucleotide loci. In certain embodiments, the altered activity comprises decreased cleavage activity as to off-target polynucleotide loci. In certain embodiments, the altered or modified activity of the modified nuclease comprises altered helicase kinetics. In certain embodiments, the modified nuclease comprises a modification that alters association of the protein with the nucleic acid molecule comprising RNA (in the case of a Cas protein), or a strand of the target polynucleotide loci, or a strand of off-target polynucleotide loci. In an aspect of the invention, the engineered CRISPR protein comprises a modification that alters formation of the CRISPR complex. In certain embodiments, the altered activity comprises increased cleavage activity as to off-target polynucleotide loci. Accordingly, in certain embodiments, there is increased specificity for target polynucleotide loci as compared to off-target polynucleotide loci. In other embodiments, there is reduced specificity for target polynucleotide loci as compared to off-target polynucleotide loci. In certain embodiments, the mutations result in decreased off-target effects (e.g., cleavage or binding properties, activity, or kinetics), such as in case for Cas proteins for instance resulting in a lower tolerance for mismatches between target and guide RNA. Other mutations may lead to increased off-target effects (e.g., cleavage or binding properties, activity, or kinetics). Other mutations may lead to increased or decreased on-target effects (e.g., cleavage or binding properties, activity, or kinetics). In certain embodiments, the mutations result in altered (e.g., increased or decreased) helicase activity, association or formation of the functional nuclease complex (e.g. CRISPR-Cas complex). In certain embodiments, as described above, the mutations result in an altered PAM recognition, i.e., a different PAM may be (in addition or in the alternative) be recognized, compared to the unmodified Cas protein. Particularly preferred mutations include positively charged residues and/or (evolutionary) conserved residues, such as conserved positively charged residues, in order to enhance specificity. In certain embodiments, such residues may be mutated to uncharged residues, such as alanine.

### Tvpe-III CRISPR-Cas Proteins

The application describes methods using Type-III CRISPR-Cas proteins. This is exemplified herein with Cas7-11, whereby a number of orthologs or homologs have been identified. It will be apparent to the skilled person that further orthologs or homologs can be identified and that any of the functionalities described herein may be engineered into other orthologs, including chimeric enzymes comprising fragments from multiple orthologs.

Computational methods of identifying novel CRISPR-Cas loci are described in EP3009511 or US2016208243 and may comprise the following steps: detecting all contigs encoding the Cas1 protein; identifying all predicted protein coding genes within 20 KB of the casl gene; comparing the identified genes with Cas protein-specific profiles and predicting CRISPR arrays; selecting unclassified candidate CRISPR-Cas loci containing proteins larger than 500 amino acids (>500 aa); analyzing selected candidates using methods such as PSI-BLAST and HHPred to screen for known protein domains, thereby identifying novel Class 2 CRISPR-Cas loci (see also Schmakov et al. 2015, Mol Cell. 60(3):385-97). In addition to the above-mentioned steps, additional analysis of the candidates may be conducted by searching metagenomics databases for additional homologs. Additionally or alternatively, to expand the search to non-autonomous CRISPR-Cas systems, the same procedure can be performed with the CRISPR array used as the seed.

In one aspect the detecting all contigs encoding the Cas1 protein is performed by GenemarkS, a gene prediction program as further described in “GeneMarkS: a self-training method for prediction of gene starts in microbial genomes. Implications for finding sequence motifs in regulatory regions.” John Besemer, Alexandre Lomsadze and Mark Borodovsky, Nucleic Acids Research (2001) 29, pp 2607-2618, herein incorporated by reference.

In one aspect the identifying all predicted protein coding genes is carried out by comparing the identified genes with Cas protein-specific profiles and annotating them according to NCBI Conserved Domain Database (CDD) which is a protein annotation resource that consists of a collection of well-annotated multiple sequence alignment models for ancient domains and full-length proteins. These are available as position-specific score matrices (PSSMs) for fast identification of conserved domains in protein sequences via RPS-BLAST. CDD content includes NCBI-curated domains, which use 3D-structure information to explicitly define domain boundaries and provide insights into sequence/structure/function relationships, as well as domain models imported from a number of external source databases (Pfam, SMART, COG, PRK, TIGRFAM). In a further aspect, CRISPR arrays were predicted using a PILER-CR program which is a public domain software for finding CRISPR repeats as described in “PILER-CR: fast and accurate identification of CRISPR repeats,” Edgar, R. C., BMC Bioinformatics, January 20; 8:18(2007), herein incorporated by reference.

In a further aspect, the case by case analysis is performed using PSI-BLAST (Position-Specific Iterative Basic Local Alignment Search Tool). PSI-BLAST derives a position-specific scoring matrix (PSSM) or profile from the multiple sequence alignment of sequences detected above a given score threshold using protein-protein BLAST. This PSSM is used to further search the database for new matches, and is updated for subsequent iterations with these newly detected sequences. Thus, PSI-BLAST provides a means of detecting distant relationships between proteins.

In another aspect, the case by case analysis is performed using HHpred, a method for sequence database searching and structure prediction that is as easy to use as BLAST or PSI-BLAST and that is at the same time much more sensitive in finding remote homologs. In fact, HHpred's sensitivity is competitive with the most powerful servers for structure prediction currently available. HHpred is the first server that is based on the pairwise comparison of profile hidden Markov models (HMMs). Whereas most conventional sequence search methods search sequence databases such as UniProt or the NR, HHpred searches alignment databases, like Pfam or SMART. This greatly simplifies the list of hits to a number of sequence families instead of a clutter of single sequences. All major publicly available profile and alignment databases are available through HHpred. HHpred accepts a single query sequence or a multiple alignment as input. Within only a few minutes it returns the search results in an easy-to-read format similar to that of PSI-BLAST. Search options include local or global alignment and scoring secondary structure similarity. HHpred can produce pairwise query-template sequence alignments, merged query-template multiple alignments (e.g. for transitive searches), as well as 3D structural models calculated by the MODELLER software from HHpred alignments.

### Deactivated/Inactivated Cas7-11 Proteins

Where the Cas7-11 protein has nuclease activity, the Cas7-11 protein may be modified to have diminished nuclease activity e.g., nuclease inactivation of at least 70%, at least 80%, at least 90%, at least 95%, at least 97%, or 100% as compared with the wild type enzyme; or to put in another way, a Cas7-11 enzyme having advantageously about 0% of the nuclease activity of the non-mutated or wild type Cas7-11 enzyme or CRISPR-Cas protein, or no more than about 3% or about 5% or about 10% of the nuclease activity of the non-mutated or wild type Cas7-11 enzyme.

### Modified Cas7-11 Enzymes

In particular embodiments, it is of interest to make use of an engineered Cas7-11 protein as defined herein, such as Cas7-11, wherein the protein complexes with a nucleic acid molecule comprising RNA to form a CRISPR complex, wherein when in the CRISPR complex, the nucleic acid molecule targets one or more target polynucleotide loci, the protein comprises at least one modification compared to unmodified Cas7-11 protein, and wherein the CRISPR complex comprising the modified protein has altered activity as compared to the complex comprising the unmodified Cas7-11 protein. It is to be understood that when referring herein to CRISPR “protein,” the Cas7-11 protein is an unmodified or modified CRISPR-Cas protein (e.g. having increased or decreased or the same (or no) enzymatic activity, such as without limitation including Cas7-11. The term “CRISPR protein” may be used interchangeably with “CRISPR-Cas protein”, irrespective of whether the CRISPR protein has altered, such as increased or decreased (or no) enzymatic activity, compared to the wild type CRISPR protein.

Computational analysis of the primary structure of Cas7-11 nucleases reveals 5 distinct domain regions.

Based on the above information, mutants can be generated which lead to inactivation of the enzyme or which modify the double strand nuclease to nickase activity. In alternative embodiments, this information is used to develop enzymes with reduced off-target effects.

In certain of the above-described Cas7-11 enzymes, the enzyme is modified by mutation of one or more residues (in the Cas7-like domains as well as the small subunit) including but not limited to positions listed in table 1.

### Orthologs of Cas7-11

The terms “orthologue” (also referred to as “ortholog” herein) and “homologue” (also referred to as “homolog” herein) are well known in the art. By means of further guidance, a “homologue” of a protein as used herein is a protein of the same species which performs the same or a similar function as the protein it is a homologue of. Homologous proteins may but need not be structurally related, or are only partially structurally related. An “orthologue” of a protein as used herein is a protein of a different species which performs the same or a similar function as the protein it is an orthologue of. Orthologous proteins may but need not be structurally related, or are only partially structurally related. Homologs and orthologs may be identified by homology modelling (see, e.g., Greer, Science vol. 228 (1985) 1055, and Blundell et al. Eur J Biochem vol 172 (1988), 513) or “structural BLAST” (Dey F, Cliff Zhang Q, Petrey D, Honig B. Toward a “structural BLAST”: using structural relationships to infer function. Protein Sci. 2013 April; 22(4):359-66. doi: 10.1002/pro.2225). See also Shmakov et al. (2015) for application in the field of CRISPR-Cas loci. Homologous proteins may but need not be structurally related, or are only partially structurally related.

The present invention encompasses the use of a Cas7-11 effector protein, derived from a Cas7-11 locus denoted as subtype III-E. Herein such effector proteins are also referred to as “Cas7-11p”, e.g., a Cas7-11 protein (and such effector protein or Cas7-11 protein or protein derived from a Cas7-11 locus is also called “CRISPR-Cas protein”).

In particular embodiments, the effector protein is a Cas7-11 effector protein from an organism from a genus comprising Candidatus Jettenia caeni, Candidatus Scalindua brodae, Desulfobacteraceae, Candidatus Magnetomorum, Desulfonema Ishimotonii, Candidatus Brocadia, Deltaproteobacteria, Syntrophorhabdaceae, or Nitrospirae.

### Example Cas Proteins

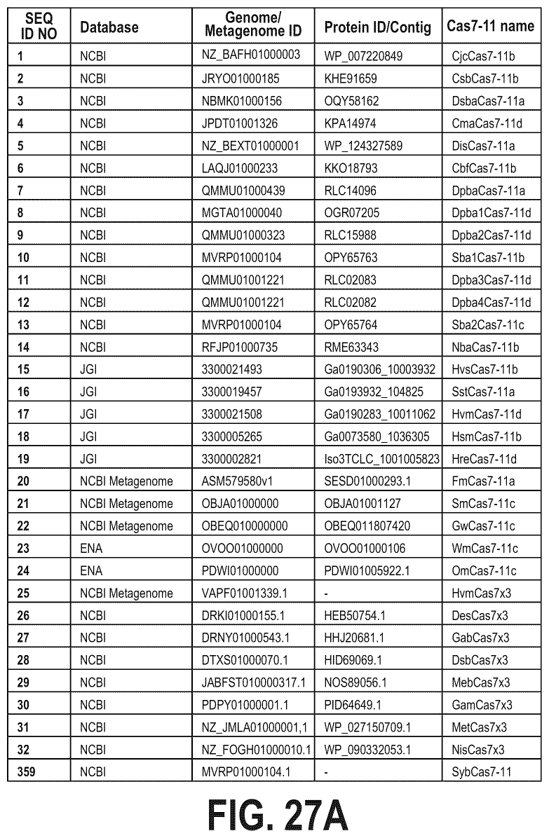

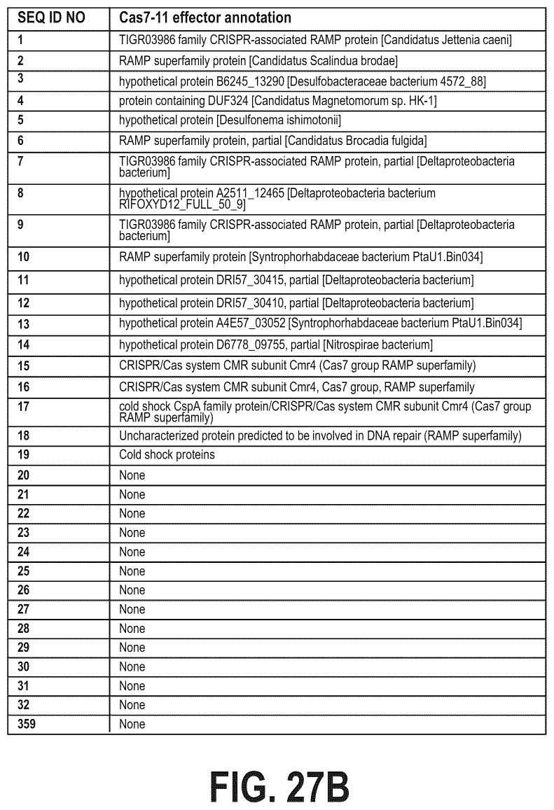

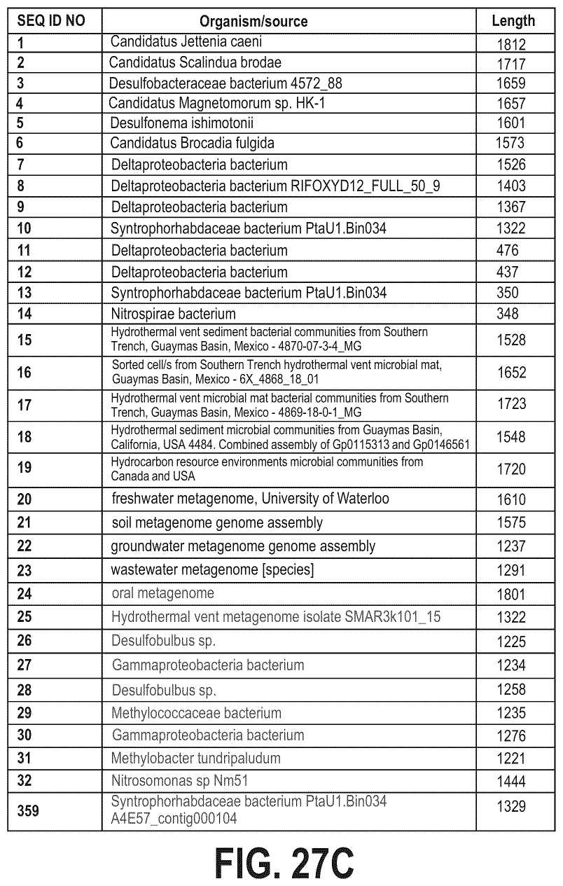

In certain example embodiments, the CRISPR effector protein is a Cas7-11 type III-D/III-E ortholog selected from Table 1 and FIG. 27A-27C.

TABLE 1

| SEQ ID NO & | |
|---|---|
| Protein | |
| ID/Contig | Sequence |
| SEQ ID NO: 1 | MHTILPIHLTFLEPYRLAEWHAKADRKKNKRYLRGMSFAQWHKDKDGIGKPYITGTL |
| WP_007220849 | LRSAVLNAAEELISLNQGMWAKEPCCNGKFETEKDKPAVLRKRPTIQWKTGRPAICD |
| | PEKQEKKDACPLCMLLGRFDKAGKRHRDNKYDKHDYDIHFDNLNLITDKKFSHPDDI |
| | ASERILNRVDYTTGKAHDYFKVWEVDDDQWWQFTGTITMHDDCSKAKGLLLASLCF |
| | VDKLCGALCRIEVTGNNSQDENKEYAHPDTGIITSLNLKYQNNSTIHQDAVPLSGSAH |
| | DNDEPPVHDNDSSLDNDTITLLSMKAKEIVGAFRESGKIEKARTLADVIRAMRLQKPD |
| | IWEKLPKGINDKHHLWDREVNGKKLRNILEELWRLMNKRNAWRTFCEVLGNELYRC |
| | YKEKTGGIVLRFRTLGETEYYPEPEKTEPCLISDNSIPITPLGGVKEWIIIGRLKAETPFY |
| | FGVQSSFDSTQDDLDLVPDIVNTDEKLEANEQTSFRILMDKKGRYRIPRSLIRGVLRRD |
| | LRTAFGGSGCIVELGRMIPCDCKVCAIMRKITVMDSRSENIELPDIRYRIRLNPYTATV |
| | DEGALFDMEIGPEGITFPFVFRYRGEDALPRELWSVIRYWMDGMAWLGGSGSTGKG |
| | RFALIDIKVFEWDLCNEEGLKAYICSRGLRGIEKEVLLENKTIAEITNLFKTEEVKFFES |
| | YSKHIKQLCHECIINQISFLWGLRSYYEYLGPLWTEVKYEIKIASPLLSSDTISALLNKD |
| | NIDCIAYEKRKWENGGIKFVPTIKGETIRGIVRMAVGKRSGDLGMDDHEDCSCTLCTI |
| | FGNEHEAGKLRFEDLEVVEEKLPSEQNSDSNKIPFGPVQDGDGNREKECVTAVKSYK |
| | KKLIDHVAIDRFHGGAEDKMKFNTLPLAGSFEKPIILKGRFWIKKDIVKDYKKKIEDA |
| | MVDIRDGLYPIGGKTGIGYGWVTDLTILNPQSGFQIPVKKDISPEPGTYSTYPSHSTPSL |
| | NKGHIYYPHYFLAPANTVHREQEMIGHEQFHKEQKGELLVSGKIVCTLKTVTPLIIPDT |
| | ENEDAFGLQNTYSGHKNYQFFHINDEIMVPGSEIRGMISSVYEAITNSCFRVYDETKYI |
| | TRRLSPEKKDESNDKNKSQDDASQKIRKGLVKKTDEGFSIIEVERYSMKTKGGTKLV |
| | DKVYRLPLYDSEAVIASIQFEQYGEKNEKRNAKIRAAIKRNEVIAEVARKNLIFLRSLT |
| | PEELKKVLQGEILVKFSLKSGKNPNDYLAELHENGTERGLIKFTGLNMVNIKNVNEED |
| | KDFNDTWDWEKLNIFHNAHEKRNSLKQGYPRPVLKFIKDRVEYTIPKRCERIFCIPVK |
| | NTIEYKVSSKVCKQYKDVLSDYEKNFGHINKIFTTKIQKRELTDGDLVYFIPNEGADK |
| | TVQAIMPVPLSRITDSRTLGERLPHKNLLPCVHEVNEGLLSGILDSLDKKLLSIHPEGLC |
| | PTCRLFGTTYYKGRVRFGFANLMNKPKWLTERENGCGGYVTLPLLERPRLTWSVPSD |
| | KCDVPGRKFYIHHNGWQEVLRNNDITPKTENNRTVEPLAADNRFTFDVYFENLREWE |
| | LGLLCYCLELEPGMGHKLGMGKPMGFGSVKIAIERLQTFTVHQDGINWKPSENEIGV |
| | YVQKGREKLVEWFTPSAPHKNMEWNGVKHIKDLRSLLSIPGDKPTVKYPTLNKDAE |
| | GAISDYTYERLSDTKLLPHDKRVEYLRTPWSPWNAFVKEAEYSPSEKSDEKGRETIRT |
| | KPKSLPSVKSIGKVKWFDEGKGFGILIMDDGKEVSISKNSIRGNILLKKGQKVTFHIVQ |
| | GLIPKAEDIEIAK |
| SEQ ID NO: 2 | MNITVELTFFEPYRLVEWFDWDARKKSHSAMRGQAFAQWTWKGKGRTAGKSFITGT |
| KHE91659 | LVRSAVIKAVEELLSLNNGKWEGVPCCNGSFQTDESKGKKPSFLRKRHTLQWQANN |
| | KNICDKEEACPFCILLGRFDNAGKVHERNKDYDIHFSNFDLDHKQEKNDLRLVDIASG |
| | RILNRVDFDTGKAKDYFRTWEADYETYGTYTGRITLRNEHAKKLLLASLGFVDKLCG |
| | ALCRIEVIKKSESPLPSDTKEQSYTKDDTVEVLSEDHNDELRKQAEVIVEAFKQNDKL |
| | EKIRILADAIRTLRLHGEGVIEKDELPDGKEERDKGHHLWDIKVQGTALRTKLKELWQ |
| | SNKDIGWRKFTEMLGSNLYLIYKKETGGVSTRFRILGDTEYYSKAHDSEGSDLFIPVTP |
| | PEGIETKEWIIVGRLKAATPFYFGVQQPSDSIPGKEKKSEDSLVINEHTSFNILLDKENR |
| | YRIPRSALRGALRRDLRTAFGSGCNVSLGGQILCNCKVCIEMRRITLKDSVSDFSEPPEI |
| | RYRIAKNPGTATVEDGSLFDIEVGPEGLTFPFVLRYRGHKFPEQLSSVIRYWEENDGK |
| | NGMAWLGGLDSTGKGRFALKDIKIFEWDLNQKINEYIKERGMRGKEKELLEMGESSL |
| | PDGLIPYKFFEERECLFPYKENLKPQWSEVQYTIEVGSPLLTADTISALTEPGNRDAIAY |
| | KKRVYNDGNNAIEPEPRFAVKSETHRGIFRTAVGRRTGDLGKEDHEDCTCDMCIIFGN |
| | EHESSKIRFEDLELINGNEFEKLEKHIDHVAIDRFTGGALDKAKFDTYPLAGSPKKPLK |
| | LKGRFWIKKGFSGDHKLLITTALSDIRDGLYPLGSKGGVGYGWVAGISIDDNVPDDFK |
| | EMINKTEMPLPEEVEESNNGPINNDYVHPGHQSPKQDHKNKNIYYPHYFLDSGSKVY |
| | REKDIITHEEFTEELLSGKINCKLETLTPLIIPDTSDENGLKLQGNKPGHKNYKFFNING |
| | ELMIPGSELRGMLRTHFEALTKSCFAIFGEDSTLSWRMNADEKDYKIDSNSIRKMESQ |
| | RNPKYRIPDELQKELRNSGNGLFNRLYTSERRFWSDVSNKFENSIDYKREILRCAGRP |
| | KNYKGGIIRQRKDSLMAEELKVHRLPLYDNFDIPDSAYKANDHCRKSATCSTSRGCR |
| | ERFTCGIKVRDKNRVFLNAANNNRQYLNNIKKSNHDLYLQYLKGEKKIRFNSKVITG |
| | SERSPIDVIAELNERGRQTGFIKLSGLNNSNKSQGNTGTTFNSGWDRFELNILLDDLET |
| | RPSKSDYPRPRLLFTKDQYEYNITKRCERVFEIDKGNKTGYPVDDQIKKNYEDILDSY |
| | DGIKDQEVAERFDTFTRGSKLKVGDLVYFHIDGDNKIDSLIPVRISRKCASKTLGGKLD |
| | KALHPCTGLSDGLCPGCHLFGTTDYKGRVKFGFAKYENGPEWLITRGNNPERSLTLG |
| | VLESPRPAFSIPDDESEIPGRKFYLHHNGWRIIRQKQLEIRETVQPERNVTTEVMDKGN |
| | VFSFDVRFENLREWELGLLLQSLDPGKNIAHKLGKGKPYGFGSVKIKIDSLHTFKINSN |
| | NDKIKRVPQSDIREYINKGYQKLIEWSGNNSIQKGNVLPQWHVIPHIDKLYKLLWVPF |
| | LNDSKLEPDVRYPVLNEESKGYIEGSDYTYKKLGDKDNLPYKTRVKGLTTPWSPWNP |
| | FQVIAEHEEQEVNVTGSRPSVTDKIERDGKMV |
| SEQ ID NO: 3 | MKITLRFLEPFRMLDWIRPEERISGNKAFQRGLTFARWHKSKADDKGKPFITGTLLRS |
| OQY58162 | AVIRAAEHLLVLSKGKVGEKACCPGKFLTETDTETNKAPTMFLRKRPTLKWTDRKGC |
| | DPDFPCPLCELLGPGAVGKKEGEAGINSYVNFGNLSFPGDTGYSNAREIAVRRVVNRV |
| | DYASGKAHDFFRIFEVDHIAFPCFHGEIAFGENVSSQARNLLQDSLRFTDRLCGALCVI |
| | RYDGDIPKCGKTAPLPETESIQNAAEETARAIVRVFHGGRKDPEQAQIDKAEQIQLLSA |
| | AVRELGRDKKKVSALPLNHEGKEDHYLWDKKAGGETIRTILKAAAEKEAVANQWR |
| | QFCIELSEELYKEAKKAHGGLEPARRIMGDAEFSDKSVPDTVSHSIGISVEKETIIMGTL |
| | KAETPFFFGIESKEKKQTDLMLLLDGQNHYRIPRSALRGILRRDIRSVLGTGCNAEVGG |
| | RPCLCPVCRIMKNITVMDTRSSTDTLPEVRPRIRLNPFTGSVQEKALFNMEMGTEGIEF |
| | PFVLSYRGKKTLPKELRNVLNWWTEGKAFLGGAASTGKSIFQLSDIHAFSSDLSDETA |
| | RESYLSNHGWRGIMENSIVHESPLEGGAGGCSFGLSDLPKLGWHAEDLKLSDIEKYKP |
| | FHRQKISVKITLNSPFLNGDPVRALTEDVADIVSFKKYTQGGEKIIYAYKSESFRGVVR |
| | TALGLRNQGNDDITGKKNVPLIALTHQDCECMLCRFFGSEYEAGRLYFEDLTFESEPE |
| | PRRFDHVAIDRFTGGAVNQKKFDDRSLVPGKEGFMTLIGCFWMRKDKELSRNEIEEL |
| | GKAFADIRDGLYPLGAKGSMGYGQVAELSIVDDEDSDDENNPAKLLAESMKNASPSL |
| | GTPTSLKKKDAGLSLRFDENADYYPYYFLEPEKSVHRDPVPPGHEEAFRGGLLTGRIT |
| | CRLTVRTPLIVPNTETDDAFNMKEKAGKKKDAYHKSYRFFTLNRVPMIPGSEIRGMIS |
| | SVFEALSNSCFRIFDEKYRLSWRMDADVKELEQFKPGRVADDGKRIEEMKEIRYPFYD |
| | RTYPERNAQNGYFRWDARISLTDNSMRKMEKDGVPRNVIYKLNTLKNKAYKSEKSF |
| | LFDLKNKAGGVGRYKKLVLKHAEVRGGEIPYYSHPTPTDCKLLSLVGPNRQLCRQDT |
| | LVQYRIIKHRRGAKPEEDFMFVGTPSENQKGHKENNDHGGGYLKISGPNKIEKENVLT |
| | SGVPSVPENMGAVVHNCPPRLVEVTVRCGRKQEEECKRKRLVPEYVCADPEKKVTY |
| | TMTKRCERIFLEKSRRIIPFTNDAVDKFEILVKEYRRNAEQQDTPEAFQTILPENGTVNP |
| | GDLLYFREEKGKAAEIVPVRISRKVDDRHIGKRIDPELRPCHGEWIEDGDLSKLDAYP |
| | AEKKLLTRHPKGLCPACRVFGTGSYKSRVRFGFAALKGTPKWLKEDPAEPSQGKGIT |
| | LPLLERPRPTWAVLHNDKENSEIPGRKFYVHHNGWKGISEGIHPISGENIEPDENNRTV |
| | EVLDKGNRFVFELSFENLEPRELGLLIHSLQLEKGLAHKLGMAKSMGFGSVEIDVESV |
| | RVKHRSGEWDYKDGETVDGWIEEGKRGVAAKGKANDLRKLLYLPGEKQNPHVHYP |
| | TLKKEKKGDPPGYEDLKKSFREKKLNRRKMLTTLWEPWHK |
| SEQ ID NO: 4 | MLKLKVKITYFQPFRVIPWIKEDDRNSDRNYLRGGTFARWHKDKKDDIHGKPYITGT |
| KPA14974 | LLRSALFTEIEKIKIHHSDFIHCCNAIDRTEGKHQPSFLRKRPVYTENKNIQACNKCPLC |
| | LIMGRGDDRGEDLKKKKHYNGKHYQNWTVHFSNFDTQATFYWKDIVQKRILNRVD |
| | QTCGKAKDFFKVCEVDHIACPTLNGIIRINDEKLSQEEISKIKQLIAVGLAQIESLAGGIC |
| | RIDITNQNHDDLIKSFFETKPSKILQPNLKESGEERFELAKLELLAEYLTQSFDANQKEQ |
| | QLRRLADAIRDLRKYSPDYLKDLPKGKKGGRTSIWNKKVADDFTLRDCLKNQKIPNE |
| | LWRQFCEGLGREVYKISKNISNRSDAKPRLLGETEYAGLPLRKEDEKEYSPTYQNQES |
| | LPKTKWIISGELQAITPFYIGHVNKTSHTRSTIFLNMNGQFCIPRSTLRGALRRDLRLVF |
| | GDSCNTPVGSRVCYCQVCQIMRCIKFEDALSDVDSPPEVRHRIRLNCHTGVVEEGALF |
| | DMETGFQGMIFPFRLYYESKNEIMSQHLYEVLNNWTNGQAFFGGEAGTGFGRFKLLN |
| | NEVFLWEIDGEEEDYLQYLFSRGYKGIETDEIKKVADPIKWKTLFTKLEIPPEKIPLTQL |
| | NYTLTIDSPLISRDPIAAMLDNRNPDAVMVKKTILVYEQDSSTHKNVPKEVPKYFIKSE |
| | TIRGLLRSIISRTEIKLEDGKKERIFNLDHEDCDCLQCRLFGNVHQQGILRFEDAEITNK |
| | NVSDCCIDHVAIDRFTGGGVEKMKFNDYPLSASPKNCLNLKGSIWITSALKDSEKEAL |
| | SKALSELKYGYASLGGLSAIGYGRVKELTLEENDIIQLTEITESNLNSQSRLSLKPDVK |
| | KELSNNHFYYPHYFIKPAPKEVVRESRLISHVQGHDTEGEFLLTGKIKCRLQTLGPLFI |
| | ANNDKGDDYFELQHNNPGHLNYAFFRINDHIAIPGASIRGMISSVFETLTHSCFRVMD |
| | DKKYLTRRVIPESETTQKRKSGRYQVEESDPDLFPGRVQKKGNKYKIEKMDEIVRLPI |
| | YDNFSLVERIREYHYSEECASYVPSVKKAIDYNRMLAQAADSNREFLYNHPEAKSILQ |
| | GKKEVYYILHKQESKNRGKTKEINPNARYACLTDENTPGSRKGFIKFTGPDMVTVNK |
| | ELKSKIAPIYDPEWEKDIPDWERSNQESNHKYSFILHNEIEMRSSQKKKYPRPVFICKK |
| | NGVEYRMQKRCERIFDFTKEEEKDKEIVIPQKVVSQYNAILKDNKENTETIPGLENSK |
| | MVNKELEDGDLVYFKYKEGKVTELTPVAISRKTDNKPMGKRFPKISINGKMKPNDSL |
| | RSCSHTCTEDCDDCPNLCESVKDYFKPHPDGLCPACHLFGTTFYKSRLSFGLAWLEN |
| | NAKWYISNDFQQKDSKKEKGGKLTLPLLERPRPTWSMPNNNAEVPGRKFYVHHPWS |
| | VENIKNNQGNQKDISLKPDSDAIKIKENNRTIEPLGKDNVFNFEISFNNLRDWELGLLL |
| | YAIELEDHLAHKLGMAKAFGMGSVKIEIKNLLIKGSINDISKAELIKKGFKKLGIDSLE |
| | KDDLSEYLHIKQLREILWFSDKPVGTIEYPKLENKTNSRIPSYTDFVQEKDHETGFKNP |
| | KYQNLKSRLHILQNPWNAWWKNEE |
| SEQ ID NO: 5 | MTTTMKISIEFLEPFRMTKWQESTRRNKNNKEFVRGQAFARWHRNKKDNTKGRPYIT |
| WP_124327589 | GTLLRSAVIRSAENLLTLSDGKISEKTCCPGKFDTEDKDRLLQLRQRSTLRWTDKNPC |
| | PDNAETYCPFCELLGRSGNDGKKAEKKDWRFRIHFGNLSLPGKPDFDGPKAIGSQRV |
| | LNRVDFKSGKAHDFFKAYEVDHTRFPRFEGEITIDNKVSAEARKLLCDSLKFTDRLCG |
| | ALCVIRFDEYTPAADSGKQTENVQAEPNANLAEKTAEQIISILDDNKKTEYTRLLADAI |
| | RSLRRSSKLVAGLPKDHDGKDDHYLWDIGKKKKDENSVTIRQILTTSADTKELKNAG |
| | KWREFCEKLGEALYLKSKDMSGGLKITRRILGDAEFHGKPDRLEKSRSVSIGSVLKET |
| | VVCGELVAKTPFFFGAIDEDAKQTDLQVLLTPDNKYRLPRSAVRGILRRDLQTYFDSP |
| | CNAELGGRPCMCKTCRIMRGITVMDARSEYNAPPEIRHRTRINPFTGTVAEGALFNME |
| | VAPEGIVFPFQLRYRGSEDGLPDALKTVLKWWAEGQAFMSGAASTGKGRFRMENAK |
| | YETLDLSDENQRNDYLKNWGWRDEKGLEELKKRLNSGLPEPGNYRDPKWHEINVSI |
| | EMASPFINGDPIRAAVDKRGTDVVTFVKYKAEGEEAKPVCAYKAESFRGVIRSAVARI |
| | HMEDGVPLTELTHSDCECLLCQIFGSEYEAGKIRFEDLVFESDPEPVTFDHVAIDRFTG |
| | GAADKKKFDDSPLPGSPARPLMLKGSFWIRRDVLEDEEYCKALGKALADVNNGLYP |
| | LGGKSAIGYGQVKSLGIKGDDKRISRLMNPAFDETDVAVPEKPKTDAEVRIEAEKVY |
| | YPHYFVEPHKKVEREEKPCGHQKFHEGRLTGKIRCKLITKTPLIVPDTSNDDFFRPADK |
| | EARKEKDEYHKSYAFFRLHKQIMIPGSELRGMVSSVYETVTNSCFRIFDETKRLSWRM |
| | DADHQNVLQDFLPGRVTADGKHIQKFSETARVPFYDKTQKHFDILDEQEIAGEKPVR |
| | MWVKRFIKRLSLVDPAKHPQKKQDNKWKRRKEGIATFIEQKNGSYYFNVVTNNGCT |
| | SFHLWHKPDNFDQEKLEGIQNGEKLDCWVRDSRYQKAFQEIPENDPDGWECKEGYL |
| | HVVGPSKVEFSDKKGDVINNFQGTLPSVPNDWKTIRTNDFKNRKRKNEPVFCCEDDK |
| | GNYYTMAKYCETFFFDLKENEEYEIPEKARIKYKELLRVYNNNPQAVPESVFQSRVA |
| | RENVEKLKSGDLVYFKHNEKYVEDIVPVRISRTVDDRMIGKRMSADLRPCHGDWVE |
| | DGDLSALNAYPEKRLLLRHPKGLCPACRLFGTGSYKGRVRFGFASLENDPEWLIPGK |
| | NPGDPFHGGPVMLSLLERPRPTWSIPGSDNKFKVPGRKFYVHHHAWKTIKDGNHPTT |
| | GKAIEQSPNNRTVEALAGGNSFSFEIAFENLKEWELGLLIHSLQLEKGLAHKLGMAKS |
| | MGFGSVEIDVESVRLRKDWKQWRNGNSEIPNWLGKGFAKLKEWFRDELDFIENLKK |
| | LLWFPEGDQAPRVCYPMLRKKDDPNGNSGYEELKDGEFKKEDRQKKLTTPWTPWA |
| SEQ ID NO: 6 | MSKTDDKIDIKLTFLEPYRMVNWLENGLRMTDPRYLRGLSFARWHRNKNGKAGRPY |
| KKO18793 | ITGTLLRSAVIRAAEELLSLNLGKWGKQLCCPGQFETEREMRKNKTFLRRRPTPAWSA |
| | ETKKEICTTHGSACAFCLLLGRRLHGGKEDVNEDAPGSCRKPVGFGNLSLPFQPTKRQ |
| | IQDVCKERVLNRVDFRTGKAQDYFRVFEIDHEDWGVYTGEITITEPRVQEMLEASLKF |
| | VDTLCGALCRIEIVGSADETKRTTSSKEGCPASTTTRDCSSSENDDTSPEDPVREDLKK |
| | IAHVIANAFQNSGNREKVHALADAIRAMRLEESSIINTLPKGKSEKTTEQIEVNKHYL |
| | WDEIPVNDTSVRHILIEQWRRWQSKKDDPEWWKFCDFLGECLYKEYKKLTSGIQSRA |
| | RVMGETEYYGALGMPDKVIPLLKSDKTKEWILVGSLKAETPFFFGLETEQTEEVEHTS |
| | LRLVMDKKGRFRIPRSVLRGALRRDMRIAFDSGCDVKLGSPLPCDCSVCQVMRSITIK |
| | DSRSEAGKLPQIRHRIRLNPFSGTVDEGALFDIEVAPEGVIFPFVMRYRGEEFPPALLSV |
| | IRYWQDGKAWLGGEGATGKGRFALAKDLKMYEWKLEDKSLHAYIDTYGHRGNEH |
| | AIGTGQGIDGFRSGSLSDLLSDISKESFRDPLASYHNYLDKRWIKVGYQITIGAPLLSAD |
| | PIGALLDPNNVDAIVFEKMKLDGDQVKYLPAIKGETIRGIVRTALGKRNNLLAKNDH |
| | DDCTCSLCAIFGNENETGKIRFEDLEVYDKDIAKKIDHVAIDRFTGGARDQMKFDTLP |
| | LIGSPERPLRLKGLFWMRRDVSPDEKARILLAFLEIREGLYPIGGKTGSGYGWVSDLEF |
| | DGDAPEAFKEMNSKRGKQASFKEKISFRYPSGAPKHIQNLKATSFYYPHYFLEPGSKV |
| | IREQKMIGHEQYYESYPSGASGEKLLSGRIICSMTTHTPLIVPDTGVIKDPENKHATYD |
| | FFQMNNAIMIPGSEIRGMISAVYEAMTNSCFRIFHEKQYLTRRISPEDKELREFIPGIVRI |
| | INGDVYIEKAEREYRLPLYDDVHIITNYEELEYEKYIKKNPGREQKIKNAHRFNKNIAR |
| | IAESNRNYLCSLDRAVRREILSGRKKVNFRLVKVNDNKNPDKEAVELCKTGPLEGLV |
| | KFSGLNAVNISNLRPGTAEEGFDAKWDMWSLNIILNRMDVRNSQKKEYPRPALHFNH |
| | DGKEYTIPKRCERVFVRAEAGKRAETEGSYKVPRKVQEQYQNILRDYESNIGHIDNTF |
| | RTLIENCGLNNGSLVYFKPDNSRKEVVAITPVKISRKTDRLPQGDRFPHTSSDLRPCVR |
| | DCLDTEGDIRMLENSPFKRLFHIHPEGLCPACQLFGTTNYRGRVRFGFASLSDGPKWF |
| | RKDEGNETCHITLPLLERPRPTWSMPDDTSTIPGRKFYVHHMGYETVKKNQRTLVKT |
| | ENNRTVKALDKENEFTFEVFFENLREWELGLLLHCLELEPEMGHKLGMGKPLGFGSV |
| | KIRIDKLQKCVVNVKDGCVLWEPEEDKIQHYIAKGLGKLTTWFGKEWDRLEHIQGLR |
| | SLQRLLPL |
| SEQ ID NO: 7 | FESYARWCKSNSGLWKPYIPGTLLRSAVLESVEYLLALIGSKNKVEICPGLYTQSENN |
| RLC14096B | PDTKYLRRRPWYELHAQKEICKTRDTACPLCLLMRTKLDNDGDGETEKNVKFGNLY |
| | PTSPLEPLQKIRPRILNRMDPGTSKARDYFRVFEIENQLCSQFRGWIWLSGDLPNMELV |
| | KSLLAAGLSNVATLAGAVCRIRIVSTDNPSMKQDLTTQDLIDDFTNYYLKGDTPPANL |
| | AASGKGDAFPRFSPGSGDHPDTTGVSHADMASSHEGTALAKDIAEKCKDILSQISASE |
| | QLRRLADIMRDLRQDSNREIMYRQVAEENHEKASLLYKKTKKGDSIAALIAGKTEGM |
| | DAETWRRLCEFLGQTFYGEAKEAGLVETPVPRILGESERYSLQKKPTVRTDLAAELVP |
| | DIEFIIKGNLIAETPFFFGTDIATETHTDLPILLTSDRHFRIPRSVLRGILRRDLRLVTGSG |
| | CSVKLGRSEPCACDVCQIMRSLTMRDCVSSCKVPPEIRHRIRLNPVTETVEEGALFDM |
| | EIGPQGISFPFVLRSRGVNSSFSTRLKNVLTWWSEGKIFMGGDKGTGKGRFTLAELEA |
| | YYFRITTKRIGKNVWVIGNYLKSQGWRGAELETHFDSLKEWKSLSFSDSDVKVFTW |
| | HKITWKVSFEGPVLTNDPIAADIRNESDAVFYQKSVAGEKGPVYALKGEGLRGIVSSS |
| | LCKKKNLSSNLHEDCECLRCKIFGSKHQEGNIRFEDMTVSQESEVREKLFDHVSIDRFT |
| | GGAANKLKFDDKPLVGNPLVFQGVFWVHQSIGNNEKTQEALSDAFKDVRDGLYPVG |
| | AKGSIGYGWIKGIEVVEGPDWLKDALSAEKTVEAGIASEESEYKLPDLPWISLLPKGR |
| | AIYNPHYFLGIPKVTPEREREPVGHDRFQTDLHTGRIICTLKTITPLIIPDTENDKAFEVE |
| | NASADHERFKFMRMGSQAAIPGSAIRSMTSSVFEALTNSCFRVLDQKSHLSWRMEAD |
| | DAGDYKPGRFEKKDDKAVIRKFKKKARFPFYAGPDTREAFTSDQIMGKEKVTLWVK |
| | DFEASLTVPDEIGWKKKRGYLKVTGPNKVEIDTENISENNPSPPDSWQDVRINDDGTIP |
| | DKKNRKFICQYGTTTYTVDKWCEAFFCDEEKDPYELAPDVERKYRLLMDSYHNNPQ |
| | APPQIFRSLPLFSETGPKKTLEHGDLVYFRLSEVNKQSQSKKQVRERVTDIVPVSISRIA |
| | NNQPIGKHIAAAFRPCAYVCIEECEPCDAKTCPIPVYREGYPIKGLCRACHLFGTTGYK |
| | GRVRFSFAKLNGDAVWAKGAGGKDYFTLPLLEKPRPTWTMPNEGAKIPGRKFYVHH |
| | NEWKTVQEGKNPIDQKAIRPNPNNSSVEVLNLGNEFQFEVSFENLEEWELGLLLYCLE |
| | LEPGLAHKLGRGKAFGFGSIEAEVSKIEMRIKSGTWKNETSGKEKFIQSGLSQVPSFFK |
| | QDEKQWNKVEQVKNIRKLLQLSWNKGNAVEPEVRYPALREKDDENKRPGYVELKD |
| | NGYDAGKKLVSPWAPWHPIKK |
| SEQ ID NO: 8 | MTKKPGTEDKATLWGKESASKSVKTILEESIQGFTVEQKRSFFANLADQLVSRAGEQ |
| OGR07205 | GAKSVRSQGLIIGRKENYAKPSAQEPTRHHLYRQPSNASAFLATGWLIAETPFFIGSGT |
| | EGQKQTDDQAESLHLRTLRDGHGRFRIPFTTIRGVMDKELRDILQAGCAKGRSLRAPC |
| | PCQVCTLMRRIQVRDAIAADILPPDLRMRTRIDPSHGTVAHLFSLEMAPQGLKLPFFLK |
| | LKGVETIDPDKELLEILNDWSAGQCFLGGLWGTGKGRFRLDDLQWHRLELDNADYY |
| | TPLLQDRFFAGETISDLRQGLQSINIQPERIPAQTPSRNMPYCRVDCILEFKSPVLSGDP |
| | VAALFESDAPDNVAYKKPVVQYDETGRLRTTDPGPVEMLTCLKGEGVRGVVAYLAG |
| | KAYDQHDLSHDSCNCTFCQAFGNGQKAGSLRFDDFMPVQFESDQAGNFSWSPHTPH |
| | AMRSDRVALDVFGGAMPEAKFDDRPLAASPGKPLNFKSTIWYREDMGKEAGKALKR |
| | ALIDLQNNMAAIGSGGGIGRGWVSRVCFEGDIPDFLEDFPEPITVTEPEQDSQLLKNQA |
| | VADETAVSACDTADAPHPLAVTLEPGARYFPRVIIPRAPTVKRDECVTGQRYHTGRLS |
| | GKIFCELNTLGPLFVPDTDYSAGVPVPISDEQLAECQLQAVFENTSKFNEFFATYPEET |
| | VTKLKDLLCAADDKWILAVKDITADLRQEIGEDTFQRIIRKAGHKTQRFHQINDEIGLP |
| | GASLRGMVLSNYQILTNSCYRNLKATEEITRRMPADEAKYRKAGRVTVSGDGAQKK |
| | YSIQEMEVLRLPIYDNMNTPDNMPDVAKQATTAKRCNNLMNEAAKTSRVELKARW |
| | REGQSKIKYQIIDALNKVDPIIQVISSSKQINPNNGKTGWGYVKYTGANVFAKSLVAPI |
| | DCLRKKDAGHVCCQVNLNPAWEASNFDILINEKCPVERQSGPRPTLRCKGQDSAWY |
| | TLTKRSERIFTDKKPVPDPINIPPREVKRYNELRDSYKKNTAHVPKPLQTFFNQESLAN |
| | GDLVYFEVNQFGEASQLTPVSISRTTDLFPIGGRLPQGHKDLFPCTAMCLSECKNCVP |
| | ASFCEFHSRSHEKLCPACSLAGTTGNRGRIKFSEAWLSGLPKWHSVSQDNVGRGLGV |
| | TMPRLERSRRTWHLPTKDAYLLGQSIYLNHPVPAILPSDQVPSENNQTVEPLGPKNIFS |
| | FQLAFDNLSIEELGLLLYSLELESGMAHRLGRGRALGMGSVQISVKDIQIRDNKSFLFS |
| | SNISKKSEWIQCGKDEFAQEAWFGESWDNIDHIQRLRQALTIPVKGDVGCIRYPKLEA |
| | EGGMPDYIKLRKRLTPLCDREEPVRYRINPVQLARMILPFVPWHGACPALLNEQVMIE |
| | AKRLTELXXXDRANWPC |
| SEQ ID NO: 9 | ASEDDDTPTLRKVLKDEINGQEDMWRKFCEALGNSLYDLSKKAKERKRTEALPRLLG |
| RLC14096 | ETEIYGLPMRENKEDEPLPSSLTYKFKWLIAGELRAETPFFFGTEVQEGQTSATILLNR |
| | DGYFRLPRSVIRGALRRDLRLVMGNDGCNMPIGGQMCECGVCRVMRHIVIEDGLSDC |
| | KIPPEVRHRIRLNCHTGTVEEGALFDMETGYQGMTFPFRLYCETENSDLDSYLWEVL |
| | NNWQNGQSLFGGDTGTGFGRFELTEPKVFLWNFSKKEKHEAYLLNRGFKGQMPVQD |
| | VKTKSFKTKTWFQIHRELDISPKKLPWYSTDYRFNVTSPLISRDPIGAMLDPRNTDAIM |
| | VRKTVFCPDPNAKNRPAPATVYMIKGESIRGILRSIVVRNEELYDTDHEDCDCILCRLF |
| | GSIHQQGSLRFEDAEVQNSVSDKKMDHVAIDRFTGGGVDQMKFDDYPLPGCPAQPLI |
| | LEGKFWVKDDIDDESKSALEKAFADFRDGLVSLGGLGAIGYGQIGDFELIGGSADWL |
| | NLPKPEENRTDVPCGDRSAQGPEIKISLDADKIYHPHFFLKPSDKNVYRERELVSHAK |
| | KKGPDGKSLFTGKITCRLSTEGPVFIPDTDLGEDYFEMQASHKKHKNYGFFRINGNVA |
| | IPGSSIRGMISSVFEALTNSCFRVFDQERYLSRSEKPDPTELTKYYPGKVKRDGNKFFIL |
| | KMKDFFRLPLYDFDFEGEAESLRPNYDEDRNEEENKGKNKNTQKVKNAVEFNIKMA |
| | GFAKHNRDFLKKYKEQEIKDIFMGKKKVYFTAGKHKPNEAHDNDKIALLTKGSNKK |
| | AEKGYFKFTGPGMVNVKAGVEGEECDFHIDESDPDVYWNMSSILPHNQIKWRPSQK |
| | KEYPRPVLKCVKDGTEYVMLKRSEHVFAEASSEDSYPVPGKVRKQFNSISRDNVQNT |
| | DHLSSMFQSRRLHDELSHGDLVYFRHDEKRKVTDIAYVRVSRTVDDRPMGKRFKNE |
| | SLRPCNHVCVEGCDECPDRCKELEDYFSPHPEGLCPACHLFGTTDYKGRVSFGLGWH |
| | ESNTPKWYMPEDNSQKGSHLTLPLLERPRPTWSMPNKKSEIPGRKFYVHHPWSVDKI |
| | RNRQFDPAKEKQPDDVIKPNENNRTVEPLGKGNEFTFEVRFNNLREWELGLLLYSLEL |
| | EDNMAHKLGMGKALGMGSARIKAEAIELRCESAGQNAELKDKAAFVRKGFEFLEID |
| | KPGENDPMNFDHIRQLRELLWFLPENVSANVRYPMLEKEDDGTPGYTDFIKQEEPST |
| | GKRNPSYLSSEKRRNILQTPWKHWYLIPPFQASAQSETVFEGTVKWFDDKKGFGFIKI |
| | NDGGKDVFVHHSSIVGTGFKSLNEGDSVAFKMGVGPKGPCAEKVKKIGN |
| SEQ ID NO: 10 | MRRQRLLGDAEYYGGTGREQPASIVISTDSDPDHKVYEWIITGQLKAETGFFFGTKAG |
| OPY65763 | AGGHTDLSILLGKDGHYRVPRSVFRGALRRDLRVAFGAGCRVEVGRERPCECPVCKV |
| | MRQITVMDTISSYREAPEIRQRIRLNPYTGTVDKGALFDMEVGPEGIEFPFVLRFRGSK |
| | SFPSELAAVIGSWTKGTAWLGGAAATGKGRFSLLGLSIHKWNLSTAEGRKSYLAAYG |
| | LRDAADKTVKRLSIDKGGKGDVGLPAGLERDALPSSVREPLWKKLVCTVDFSSPLLL |
| | ADPIAALLGVEGDERIGFDNIAYEKRRYNGETNTTESIPAVKGETFRGIVRTALGKRHG |
| | NLTRDHEDCRCRLCAVFGKEQEAGKIRFEDLMPVGAWTRKHLDHVAIDRFHGGAEE |
| | NMKFDTYALAASPTNPLRMKGLIWVRSDLFETGHDGPTPPYVKDIIDALADVKRGLY |
| | PVGGKTGSGYGWIKDVTIDGLPQGLSLPPAEERVDGVNEVPPYNYSAPPDLPSAAEGE |
| | YFFPHVFIKPYDKVDRVSRLTGHDRFRQGRITGRITCTLKTLTPLIIPDSEGIQTDATGH |
| | KMCKFFSVAGKPMIPGSEIRGMISSVYEALTNSCFRVFDEEKYLTRRVQPKKGAKSSE |
| | LVPGIIVWGQNGGLAVQQVKNAYRVPLYDDPAVTSAIPTEAQKNKERWESVPSVNL |
| | QGALDWNLTTANIARDNRTFLNSRPEEKDAILSGTKPISFELEGTNPNDMLVRLVPDG |
| | VDGAHSGYLKFTGLNMVLKANKKTSRKLAPSEEDVRTLAILHNDFDSRRDWRRPPNS |
| | QRYFPRSVLRFSLERSTYTIPKRCERVFEGTCGEPYSVPSDVERQYNSIIDDISKNYGRIS |
| | ETYLTKTANRKLTVGDLVYFIADLDKNMATHILPVFISRISDEKPLGELLPFSGKLIPCE |
| | GEPPTILKKMAPSLLTEAWRTLISTHLEGFCPACRLFGTTSYKGRIRFGFAEHTGTPKW |
| | LREELDWARPFLTLPIQERPRPTWSVPDDKSEVPGRKFYLHHHGGNRIVESNLRNRPE |
| | VNQTKNNSSVEPISAGNTFTFDVCFENLEAWELGLLLYCLELSPKLAHKLGRAKAFGF |
| | GSVKIHVERIEERTTDGAYQDVTAVKKNGWITTGHDKLREWFHRDDWEDVDHIRNL |
| | RTVLRFPDADQEHDVRYPELKANNGVSGYVELRDKMTASERQESLRTPWYRWFPQN |
| | GTGGSGRHEQAATSQEQDTAKDESVLSATQRRQAVIDVSDPDERLSGTVESFDRQKG |
| | DGYIGCGVRQFYVRLEDIRSRTALCEGQVVTFRARKEWEGHEAYDVEIDQ |
| SEQ ID NO: 11 | MLEKALADFRDGVVSLGGLGAIGYGRIGDFEVAEESGTWLKIPEKKLPEDSVQCGER |
| RLC02083 | YRFSSDPATRFEKEKIYYPHYFLKPSDEDVRRETRLVSHVYQEDTDGKTRLLTGTIRC |
| | RLTTEGPIFIPDTDDPKEDYFQMEIEGHKSYGFFRINEQVAIPGSSIRGMVSSVFEALTN |
| | SCFRVFDQKRYLSRSTKPDPRELEKYLPGKVKRIDNKWVLLELEDIFRLPLYDLKDVG |
| | PKSLDSAYGLEKFKNEKRFRLKKIENAVAFNKKMAGYAKHTREFLKNNYTETELGKI |
| | LRGEMKVWFTIGHKPNSAHDNDKIALLTKKTNKRAKSGFIKFTGPSMVNIKADASNS |
| | DCEECRFDMKSEDKDGLIFHNAIECRPSQKKEYPRPVLKCVKEAVEYTMIKRCEQVFS |
| | EGKKPPRSYSIPDKSRRQYNGILKDNRDNTEHIPSFFRNRMKNKELSDEDLVYFRYKG |
| | KKVTNIAPVRVSR |
| SEQ ID NO: 12 | QYNLPLNPDAFPKFRWIVTGHLRAETPFFFGRGEIKDRTIEEETEQTSKTILLNKDNFFR |
| RLC02082 | LPRSVIRGALRRDLRLVIGNGCNTPVGGKFCECDVCRIMRHVVVEDTISSCRKPPEIRY |
| | NIRLNGHTRTVEEGALFDTETGYQGMRFPFRLCFETRASEFDPDTSEPIPKFDPYLSEV |
| | MKHWKAGQAVFGGDTGAGFGRFRLEGDIRFFSMDVAKKEEYDPYLLARGFKGMSS |
| | QEILEKIGSGRTYDWNSVPKIALNIPPNKLPWKEICYTIEVISPLISRDPIRAMMDPRNT |
| | DTIMIRKRVFVPDGKGGTLPEPESRYFIKSETLRGILRSLVGGNKTADGEYLCDLDHED |
| | CDCVQCRLFGSIHQQGCLRFEDAEVWNSVRDKKMDHVAIDRFTGGALDQMKFDDYP |
| | LLGCPEYPVILGGRFWIRDDISDKEKEIA |
| SEQ ID NO: 13 | MESIPVTLTFLEPYRVVEWYANEDRRSAERYLRGQSFARWHRKKNDKKGRPYITGTL |
| OPY65764 | LRSAAIRAAEELLSLSGGVWDGQHCCKGQFLSGGVKPEYMRKRPTYIWAEKEGACS |
| | APDYCPFCIFLGDRDQAEKKAESQNGYPDKSYHIRFGNLSLPDPPPLLDLKEVAVERT |
| | LNRVDFQTAKAHDYFKVWEISHEDLGVYTGQIVIHYTGPWQEKVKSLLEGSLRFVDR |
| | LCGALCKIEMAPKPARPLPKSLSVDMTEHAKIIVTAFDDAKKAEKVRGLADAMRSMG |
| | SKGPTILDKLPAGHDDRDHHTWDVTIVDKTPLRTYLKGVLRADDAASWPALCKALG |
| | NALYDVSQG |
| SEQ ID NO: 14 | EKQGFRDKGFNIVGSLKDAIGKEIGLREISLRPAKEETMPRWQCVEYTIIVNSPLHTAD |
| RME63343 | PIEALLHSGNYDSVVYKKTVVRNGNIKQIPVFKGETIRGIVRTAFARILRTENVEFDEE |
| | HEDCTCPLCQVFGNEHRAGRVRFEDLVIEGYTSEKKFDHVSIDRFTGGAAEKRKFDD |
| | LSLKGSPRRPIVLRGKVWIRNDMDSKGIEKLKQAFMDIRDGLYPLGSRGGIGYGWVT |
| | DLKIENTEVEEFRLDKVSTTEGSGPATEEFNFPSLPEIQLNKDAVYHPHYFIRPHEKVN |
| | REIRPVGHERFHDDLLTGRIKCTLKTLTPLIIPDTEDPDAFGLQAEHKGHQNFRFFR |
| SEQ ID NO: 15 | MKSIPITLTFLEPYRILPWAEKGKRDKKEYLRGANYVRLHKDKNGKFKPYITGTLIRS |
| Ga0190306_ | AVLSAIEMLLDITNGEWNGKECCLAKFHTEGEKPSFLRKKPIYIRAEKDEICTSRETAC |
| 10003932 | PLCLILGRFDKAEKKEKDKEKFDVHFSNLNLYSSKEFSTIEELAPKRALNRIEQYTGKA |
| | QDYFTVYEALNKEFWTFKGRIRIKEDIYDKVTDLLFSALRCVEKIAGALCRIEIDKEPS |
| | QQKGFVKRQLSKQAKEDIEKIFQVVKDAQKLRLLSDCFRELTRMANKDELALPLGPE |
| | DDGHYLWDKIKVEGKTLRIFLRNCFSQYKDNWLCFCDEASKKGYQKYREKRHKLTD |
| | RELPTATPKHFAEKKDPQISPIYIDKDDKVYEWIIVGRLIAQTPFHFGDEEKAEGAILLT |
| | PDNRFRLPRTALRGILRRDLKLAGASACEVEVGRSEPCPCDVCKIMRRVTLLDTVSED |
| | LRDFLPELRKRIRINPQSGTVAEGALFDTEVGPEGLSFPFVLRYKCEKLPDSLTTVLCW |
| | WQEGLAFLSGESATGKGRFRLEINGAFVWDLQKGLFNYIKNHGFRGEERLFLEGNEA |
| | ELEKMGIQINTELLQPEMIKKEKNFTDFPYDLIKYQLNISSPLLLNDPIRAIALYEGEGK |
| | APDAVFFKKYVFENGKIEEKPCFKAESIRGIFRTAVGRIKNVLTKNHEDCICVLCHLFG |
| | NVHETGRLKFEDLKIVSGQEEKFFDHVAIDRFLGGAKEKYKFDDKPIIGAPDTPIVLEG |
| | KIWVKKDINDEAKETLSQAFSDINTGIYYLGANGSIGYGWIEEVKALKAPSWLKIKEK |
| | PNFEKDTSLNISAIMNEFKKDIQTLNLDKTYLPYGFLKLLEKVKRTSSPITHERFYENH |
| | LTGFIECSLKVLSPLIIPDTETPEKEENGHKYYHFLKIDNKPIIPGAEIRGAVSSIYEALTN |
| | SCFRVFGEKKVLSWRMEGKDAKEFMPGRVSKKKGKLYMVKMQALRLPVYDNPALA |
| | NEIRSGSIYEKYKNSKVEIIFFQTVEGIRKFLRGNFNNVEWKKVLVTGIDPLAILPSQKIP |
| | GNDKWVKNLQSKISPVRGYFKFTGPNKIETKRREEEKDEKLRTKANKVSCLQKDKW |
| | YEAMHNHVEYKQDYTPPNSPKTEPLERPRNIPCFVCSDKEKIYRMTKRCERVFVSLGE |
| | NAPKYEIPISAIKRYEVILSAYRENWERNKTPELFRTRLPGDGRTLNEDDLVYFRADEN |
| | EKVKDIIPVCISRIVDEVPLIKRLSQELWPCVLAECPLLGFECKKCELEGLPEKIWFRIN |
| | KDGLCPACRLFGTQIYKSRVRFSFAYAKNWKFYDGYITLPRLESPRATWLILKEKDKH |
| | YIKYKVCGRKFYLHNSTYEDIINNSKKEKEKKTENNASFEVLKEGEFTFKVYFENLEN |
| | WELGLLLLSLTGLGEAIKIGHAKPLGFGSVKIEAKKIYFREEAGKFHPCEKADEYLKK |
| | GLNKLTSWFGKNEINEHMRNLLLFMTYYQNLPKVKYPDFDGYAKWRCSYVEQDKV |
| | EYFQNRWIVAS |
| SEQ ID NO: 16 | MIINITVKFLGPFRMLEWTDPDNRNRKNREFMRGQAFARWHNSNPQKGSQPYITGTL |
| Ga0193932_ | VRSAVIRSAENLLMLSEGKVGKEKCCPGEFRTENRKKRDAMLHLRQRSTLQWKTDK |
| 104825 | PLCNGKSLCPICELLGRRIGKTDEVKKKGDFRIHFGNLTPLNRYDDPSDIGTQRTLNRV |
| | DYATGKAHDFFKVWEIDHSLLSVFQGKISIADNIGDGATKLLEDSLRFTDRLCGAICVI |
| | SYDCIENSDGKENGKTGEAAHIMGESDAGKTDAENIANAIADMMGTAGEPEKLRILA |
| | DAVRALRIGKNTVSQLPLDHEGKENHHLWDIGEGKSIRELLLEKAESLPSDQWRKFCE |
| | DVGEILYLKSKDPTGGLTVSQRILGDEAFWSKADRQLNPSAVSIPVTTETLICGKLISET |
| | PFFFGTEIEDAKHTNLKVLLDRQNRYRLPRSAIRGVLRRDLRTAFGGKGCNVELGGRP |
| | CLCDVCRIMRGITIMDARSEYAEPPEIRHRIRLNPYTGTVAEGALFDMELGPQGLSFDF |
| | ILRYRGKGKSIPKALRNVLKWWTKGQAFLSGAASTGKGIFRLDDLKYISFDLSDKDK |
| | RKDYLDNYGWRNRIEALSLEKMPLDRMNDYAEPLWQKVSVEIEIGSPFLNGDPIRALI |
| | EKDGSDIVSFRKYADDSGKEVYAYKAESFRGVVRAALARQHFDKEGKPLDKEGKPL |
| | LTLIHQDCECLICRLFGSEHETGRLRFEDLLFDPQPEPMIFDHVAIDRFTGGAVDKKKF |
| | DDCSLPGTPGHPLTLKGCFWIRKELEKPDEDKSEREALSKALADIHNGLYPLGGKGAI |
| | GYGQVMNLKIKGAGDVIKAALQSESSRMSASEPEHKKPDSGLKLSFDDKKAVYYPH |
| | YFLKPAAEEVNRKPIPTGHETLNSGLLTGKIRCRLTTRTPLIVPDTSNDDFFQTGVEGH |
| | ESYAFFSVNGDIMLPGSEIRGMLSSVYEALTNSCFRVFDEGYRLSWRMEADRNVLMQ |
| | FKPGRVTDNGLRIEEMKEYRYPFYDRDCSDKKSQEAYFDEWERSITLTDDSLEKMAE |
| | RKGDISPKDLKVLKSLKGKNYKSTEGLLAAFKDKGGDTGGNILGLIFKYAERIGDVPR |
| | YEHPTDTDRMMLSLSEYNRNQKSDGKRAYKIIKPASKLGKGAYFMFAGTSVENKRIC |
| | NPACTDKANKSVKGYLKISGPNKLEKYNISEPELDGVPEDRNCQIIHNRIYLRKIFVAN |
| | AKKRKERDRLVGEFACYDPEKKVTYSMTKRCERIFIKDRGRTLPITHEASELFEILVQE |
| | YRENAKRQDTPEVFQTLLPDNGRLNPGDLVYFREEKGKTVEIIPVRISRKIDDSPIGKR |
| | LREDLRPCHGEWIEGDDLSQLSEYPEKKLFTRNTEGLCPACRLFGTGAYKGRLRFGFA |
| | KLENDPKWLMKNSDGPSHGGPLTLPLLERPRPTWSMPDDTLNRLKKDGKQEPKKQK |
| | GKKGPQVPGRKFYVHHDGWKEINCGCHPTTKENIVQNQNNRTVEPLDKGNTFSFEIC |
| | FENLEPYELGLLLYTLELEKGLAHKLGMAKPMGFGSIDIEVENVSLRTDSGQWKDAN |
| | EQISEWTDKGKKDAGKWFKTDWEAAEHIKNLKKLLFLPGEEQNPRVIYPALKQKDIP |
| | NSRLPGYEELKKNLNMEKRKEMLTTPWAPWHPIKK |
| SEQ ID NO: 17 | MTQITIQVTFFHPFRVVPWNHRDHRKTDRKYLRGGTFAKWHCTASEGKSGRPYITGT |
| Ga0190283_ | LLRSALFAEIEKLIAFHDPFKCCRGKDKTENGNAKPLFLRRRPRADCDPCGTCPLCLL |
| 10011062 | MGRSDTVRRDAKKQKKDWSVHFCNLREATERSFNWKETAIERIVNRVDPSSGKAKD |
| | YMRIWEIDPLVCSQFNGIITINLDTDNAGKVKLLMAAGLAQINILAGSICRADIISEDHD |
| | ALIKQFMAIDVREPEVSTSFPLQDDELNNAPAGCGDDEISTDQPVGHNLVDRVRISKIA |
| | ESIEDVESQEQKAQQLRRMADAIRDLRRSKPDETTLDALPKGKTDKDNSVWDKPLKK |
| | DILPSPRMPASEDDDTPTLRKVLKDEINGQEDMWRKFCEALGNSLYDLSKKAKERKR |
| | TEALPRLLGETEIYGLPMRENKEDEPLPSSLTYKFKWLIAGELRAETPFFFGTEVQEGQ |
| | TSATILLNRDGYFRLPRSVIRGALRRDLRLVMGNDGCNMPIGGQMCECGVCRVMRHI |
| | VIEDGLSDCKIPPEVRHRIRLNCHTGTVEEGALFDMETGYQGMTFPFRLYCETENSDL |
| | DSYLWEVLNNWQNGQSLFGGDTGTGFGRFELTEPKVFLWNFSKKEKHEAYLLNRGF |
| | KGQMPVQDVKTKSFKTKTWFQIHRELDISPKKLPWYSTDYRFNVTSPLISRDPIGAML |
| | DPRNTDAIMVRKTVFCPDPNAKNRPAPATVYMIKGESIRGILRSIVVRNEELYDTDHE |
| | DCDCILCRLFGSIHQQGSLRFEDAEVQNSVSDKKMDHVAIDRFTGGGVDQMKFDDYP |
| | LPGCPAQPLILEGKFWVKDDIDDESKSALEKAFADFRDGLVSLGGLGAIGYGQIGDFE |
| | LIGGSADWLNLPKPEENRTDVPCGDRSAQGPEIKISLDADKIYHPHFFLKPSDKNVYRE |
| | RELVSHAKKKGPDGKSLFTGKITCRLSTEGPV FIPDTDLGEDYFEMQASHKKHKNYGF |
| | FRINGNVAIPGSSIRGMISSVFEALTNSCFRVFDQERYLSRSEKPDPTELTKYYPGKVKR |
| | DGNKFFILKMKDFFRLPLYDFDFEGEAESLRPNYDEDRNEEENKGKNKNTQKVKNAV |
| | EFNIKMAGFAKHNRDFLKKYKEQEIKDIFMGKKKVYFTAGKHKPNEAHDNDKIALLT |
| | KGSNKKAEKGYFKFTGPGMVNVKAGVEGEECDFHIDESDPDVYWNMSSILPHNQIK |
| | WRPSQKKEYPRPVLKCVKDGTEYVMLKRSEHVFAEASSEDSYPVPGKVRKQFNSISR |
| | DNVQNTDHLSSMFQSRRLHDELSHGDLVYFRHDEKRKVTDIAYVRVSRTVDDRPMG |
| | KRFKNESLRPCNHVCVEGCDECPDRCKELEDYFSPHPEGLCPACHLFGTTDYKGRVSF |
| | GLGWHESNTPKWYMPEDNSQKGSHLTLPLLERPRPTWSMPNKKSEIPGRKFYVHHP |
| | WSVDKIRNRQFDPAKEKQPDDVIKPNENNRTVEPLGKGNEFTFEVRFNNLREWELGL |
| | LLYSLELEDNMAHKLGMGKALGMGSARIKAEAIELRCESAGQNAELKDKAAFVRKG |
| | FEFLEIDKPGENDPMNFDHIRQLRELLWFLPENVSANVRYPMLEKEDDGTPGYTDFIK |
| | QEEPSTGKRNPSYLSSEKRRNILQTPWKHWYLIPPFQASAQSETVFEGTVKWFDDKKG |
| | FGFIKINDGGKDVFVHHSSIVGTGFKSLNEGDSVAFKMGVGPKGPCAEKVKKIGN |
| SEQ ID NO: 18 | MTKIPISLTFLEPFRLVDWVSESERDKSEFLRGLSFARWHRIKNQREDENQGRPYITGT |
| Ga0073580_ | LLRSAVIKAAEELIFLNGGKWQSEECCNGQFKGSKAKYRKVECPRRRHRATLKWTD |
| 1036305 | NTCSDYHNACPFCLLLGCLKPNSKENSDIHFSNLSLPNKQIFKNPPEIGIRRILNRVDFT |
| | TGKAQDYFYVWEVEHSMCPKFQGTVKINEDMPKYNVVKDLLISSIQFVDKLCGALC |
| | VIEIGKTKNYICQSFSSNIPEEEIKKLAQEIRDILKGEDALDKMRVLADTVLQMRTKGP |
| | EIVNELPRGIEKKGGHWLWDKLRLRKKFKEIANNYKDSWQELCEKLGNELYISYKEL |
| | TGGIAVKKRIIGETEYRKIPEQEISFLPSKAGYSYEWIILGKLISENPFFFGKETKTEEQID |
| | MQILLTKDGRYRLPRSVLRGALRRDLRLVIGSGCDVELGSKRPCPCPVCRIMRRVTLK |
| | DARSDYCKPPEVRKRIRINPLTGTVQKGALFTMEVAPEGISFPFQLRFRGEDKFHDAL |
| | QNVLVWWKEGKLFLGGGASTGKGRFKLEIEHVLKWDLKNNFHSYLQYKGLRDKGD |
| | FNSIKEIEGLKVETEEFKVKKPFPWSCVEYTIFIESPFVSGDPVEAVLDSSNTDLVTFKK |
| | YKLEESKEVFAIKGESIRGVFRTAVGKNEGKLTTENEHEDCTCILCRLFGNEHETGKV |
| | RFEDLELINDSAPKRLDHVAIDRFTGGAKEQAKFDDSPLIGSPDSPLEFTGIVWVRDDI |
| | DEEEKKALKSAFLDIKSGYYPLGGKKGVGYGWVSNLKIESGPEWLRLEVQEKSSQEN |
| | VLSPVILSEVMDIEFNPPKIDENGVYFPYAFLRPLNEVKRTREPIGHNEWKKSLISGYLT |
| | CRLELLTPLIIPDTSEEVIKEKVNNGEHPVYKFFRLGGHLCIPAAEIRGMISSVYEALTN |
| | SCFRVFDEKRLISWRMTAEEAKRPDPKKSEEQNRMRFRPGRIIKKDKKFYAQEMLEL |
| | RIPVYDNKDKRNEISQNDPTRPSEYNHPTEPERIFFSNAEKIRNFLKRNSNYLHGSTPLL |
| | FRQWSISNRYDKIALIGNKSQGHLKFTGPNKIEVSEGTKCPKYETIPGRDEWDKAVHN |
| | YVEPGKFVTVISRKKGQKPKAVQRRRNVPAFCCYDYNTNRCFVMNKRCERVFKVSR |
| | DKPKYEIPPDAIRRYEHVLRKYRENWERYDIPEVFRTRLPGDGETLNEGDLVYFRLDE |
| | NNRVLDIIPVSISRISDTQYLGRRLPDHLRSCVRECLYEGWGDCKPCKLSLFPEKMWIR |
| | INPEGLCPACHLFGTQVYKGRVRFGFARAGSNWKFREEQLTLPRFETPRPTWVIPKRK |
| | DEYQIPGRKFYLHHNGWEEIYKKNKKNEIKKEKNNATFEVLKQGTFYFKVFFENLEL |
| | WELGLLIFSAELGGEEFAHKLGHGKALGFGSVKISVDKIILRRDPGQFEQRGQKFKRD |
| | AVDKGFCVLENRFGKTNFKIYLNNFLQLLYWPNNKKVKVRYPYLRQEDDPEKLPGY |
| | VELKKHQMLKDDNRYSLFARPRAVWLKWTEMVQRDKS |
| SEQ ID NO: 19 | MSVEEFYVRLTFLEPFRVVPWVRNGDERKGDRIYQRGGTYARWHKINDSHGQPYITG |
| Iso3TCLC_ | TMLRSAVLREIENTLTLHNTYGCCPGGTRTTEGKLEKPLYLRRRDGFEFENHAEKPCS |
| 1001005823 | EEDPCPLCLIQGRFDKLRRDEKKQFVRQGNISFCSVNFSNLNISSGIKSFSWEEIAVSRV |
| | VNRVDPNSGKAKDFFRVWEIDHKLCPNFLGKMSISLSEKLEDVKALLAVGLAQVNVL |
| | SGALCRVDIIDPETQKDTVHQHLIQQFVTRIQDKEKGDAADIPAFTLPPAGLSPSSNEW |
| | NDTIKSLAEKIRKIKELEQGQKLRQMADVIRELRRKTPAYLDQLPAGKPEGRESIWEK |
| | TPTGETLTLRQLLKSANVPGESWRAFCEELGEQLYRLEKNLYSHARPLPRLLGETEFY |
| | GQPARKSDDPPMIRASYRAFPSYVWVLDGILRAETPFYFGTETSEGQTSQAIILCPDGS |
| | YRLPRSLLRGVIRRDLRAILGTGCNVSLGKVRPCSCPVCEIMRRITVQQGVSSYREPAE |
| | VRQRIRSNPHTGTVEEGALFDLETGPQGMTFPFRLYFRTRSPYIDRALWLTINHWQEG |
| | KAIFGGDIGVGMGRFRLENLQIRSADLVSRRDFSLYLRARGLKGLSREEVTRIGLNEE |
| | QWEAVMADDPGTHYNPFPWEKISYTLLIHSPLISNDPIAAMLDHDNKDAVMVQKTVL |
| | FVDESGNYSQMPHHFLKGSGIRGACRFLLGRKDAPNENGLTYFEADHEECDCLLCSL |
| | FGSKHYQGKLRFEDAELQDEVEAIKCDHVAIDRFHGGTVHRMKYDDYPLPGSPNRPL |
| | RIKGNIWVKRDLSDTEKEAVKDVLTELRDGLIPLGANGGAGYGRIQRLMIDDGPGWL |
| | ALPERKEDERPQPSFSPVSLGPVHVNLKSGSDTADVYYYHPHYFLEPPSQTVSRELDII |
| | SHARTRDSGGEALLTGRILCRLITRGPIFIPDTNNDNAFGLEGGIGHKNYRFFRINDELA |
| | IPGSELRGMVSSVYEALTNSCFRIMEEGRYLSRRMGADEFKDFHPGIVVDGAKIREMK |
| | RYRLPLYDTPDKTSRTKEMTCPELFTRKDGRPERAKKFNEEIAKVAVQNRAYLLSLD |
| | EKERREVLLGNREVTFDECPDDEYSDDEYSELKYAQKYKDFIAVLKKNGQKRGYIKF |
| | TGPNTANKKNEDAPDKNYRSDWDPFKLNILLESDPECRVSNIHCYPRPLLVCIKDKAE |
| | YRIHKRCEAIFCSIGSPSDLYDIPQKVSNQYRTILQDYNDNTGKIVEIFRTQIKHDQLTT |
| | GDLVYFKPAANGQVNAVIPVSISRKTDENPLAKRFKNDSLRPCAGLCVEDCNECPAR |
| | CKKVADYFNPHPRGLCPACHLFGTTFYKGRVRFGFAWLTGEDGAPRWYKGPDPCDS |
| | GKGRPMTIPLLERPRPTWSIPDNSFDIPGRKFYVHHPYSVDGIDGETRTPNNRTIEPLAE |
| | GNEFVFDIDFENLRDWELGLLLYSLELEDSLAHKLGLGKPLGFGTVQINIRGISLKNGS |
| | KGWDTKTGDDKNQWIKKGFAHLGIDIKEANERPYIKQLRELLWVPTGDNLPHVRYPE |
| | LESKTKDVPGYTSLLKEKDLADRVSLLKAPWKPWKPWSGTAPHPDKGTNRLRASIVE |
| | RDRIQRKTDTAKPEKKEETKVGKSSSSDIEKRYVGTVKWFNDKKGYGFILYGTDEEIF |
| | VHRSGVADNSIPKEGQKVGFRIERGARGSHAVEVKAIE |
| SEQ ID NO: 20 | MPRFQLSLTFFDEPFRLIEWTDKSNRNSANTQWMRGQGFARWHKITLEKGFPFVTGT |
| SESD01000293.1 | AVRSKIIREVEALLSRNKGTWNGIPCCSGFFDTKGPSPTHLRYRPTLEWEYGKTVCTSE |
| | ADVCPLCLLLGRFDQAGKKSDTPCQSTDYHVHWENLSAGVAQYRLEDIAQKRTSNR |
| | VDFFSKKAHDHYGVWEVTAVKNLLGYIYISDAITESHQKTVISLLKAALSFTDTLCGA |
| | NCKLELSDEPVDSIHSNQSASNFNPHSGAAPSQCSQSMPPFNMDQETKELANTLCKAF |
| | TGNMRHLRTLADAVREMRRMSPGISSLPRGRLNKEGEITAHYLWDERIDEKTIRQVLE |
| | DTIELSPARSIIYKNWISFCNQLGQKLYERAKDNDPILERKRPLGEAAFSKVPTSSHAPR |
| | HDMNSRVKGGFTREWIIVGTLRALTPFYMGTGSQAGKQTSMPTLQDSNDHFRLPRTA |
| | LRGALRRDINQASDGMGCVVELGPHNLCSCPVCQVLRQIRLLDTKSKFSMPPAIRQKI |
| | CKNPVLSIVNEGSLFDVELGIEGETFPFVMRYRGGAKIPDTIITVLSWWKNERLFIGGE |
| | SGTGRGRFVLECPRIFCWDVEKGQNDYIQYHGFRNKEDELLSVYSTVSGLAEKNDVN |
| | LNNARDFSFDKICWEVQFDGPVLTGDPLAALFHGNTDSVFYKKPILKSGEKEPSYQW |
| | AIKSDTVRGLIRSAFGKRDALLIKSHEDCDCLLCEAFGSKHHEGKLRFEDLTPKSDEIK |
| | TYRMDHVAIDRISGGAVDQCKYDDEPLVGTSKHPLVFKGMFWINRDSSVEMQRALIA |
| | AFKEIRDGLYPLGSNGGTGYGWISHLAITNGPDWLNLEEVPLPQPTADIPVEECTAEPY |
| | PKFQKPDLDQNAVYYPHYFLQPGKPAERERHPVSHDHIDDKLLTGRLVCTLTTKTPLI |
| | IPDTQTNTMLPPNDAPEGHKSFRFFRIDDEVLIPGSEIRGMVSTVFEALTGSCFRVINQK |
| | AHLSWRINADMAKHYRPGRIIQNNEKMFIQPYKMFRLPFYAGFDPRNCLSEKQLLGIE |
| | PVKLWVKDFVASLVKPQTDIDIEWKEKIGFVRVTGPNKVEVDSSNTPDPSLPECESDW |
| | KDIHITEDGSTPSKNDRVYRCQLKGVTYTVAKWCEAFWVKDEGKKPITVNAEAINRY |
| | HLIMKSYQDNPQSPPIIFRSLPVLNYKQDQKIIGSMIFYRESAKSDKIVNEIIPVKISRTA |
| | DTELLAKHLPNNDFLPCAATCLNECDTCNAKTCKFLPLYREGYPVNGLCPSCHLFGTT |
| | GYQGRVRFGFAKMNGNAKFCQGGERPEDRAVTLPLQERPKLTWVMPNENSTIPGRK |
| | FFLHHQGWKKIVDEGKNPINGDVIEPDANNRTVEPLAAGNDFSFEVFFENLREWELGL |
| | LRYTLELESELAHKLGMGKAFGFGSVKIKIKSVDLRKQGEWEKATNTLVSEDKKSSW |
| | YNIHTVNNLRTALYYVEDDKIQVNYPKLKKDNESDNRPGYVEMKKTAFPVRDILTTP |
| | WWPWWPPTPPPMNQSGNQSYARSEEPARITESQPEVYKTGTVKFYKHDKKFGFITM |
| | DGRENIHFAGNQICRPETSLQSGDKVKFIEGENYKGPTALKVERLKG |
| SEQ ID NO: 21 | MRLKINIHFLEPFRLIEWHEQDRRNKGNSRWQRGQSFARWHRRKDNDQGRPYITGTL |
| OBJA01001127 | LRSVVIRAVEEELARPDTAWQSCGGLFITPDGQTKPQHLRHRATVRARQTAKDKCAD |
| | RQSACPFCLLLGRFDQVGKDGDKKGEGLRFDVRFSNLDLPKDFSPRDFDGPQEIGSRR |
| | TINRVDDETGKAHDFFSIWEVDAVREFQGEIVLAADLPSRDQVESLLHHALGFVDRLC |
| | GARCVISIADQKPAEREERTVAAGDEKATIADYDQVKGLPYTRLRPLADAVRNLRQL |
| | DLAELNKPDGKFLPPGRVNKDGRRVPHYVWDIPLGKGDTLRKRLEFLAASCEGDQA |
| | KWRNICESEGQALYEKSKKLKDSPAAPGRHLGAAEQVRPPQPPVSYSEESINSDLPLA |
| | EWIITGTLRAETPFAIGMDAPIDDDQTSSRTLVDRDGRYRLPRSTLRGILRRDLSLASG |
| | DQGCQVRLGPERPCTCPVCLILRQVVIADTVSETTVPADIRQRIRRNPITGTAADGGLF |
| | DTERGPKGAGFPFSLRYRGHAPMPKALRTVLQWWSAGKCFAGSDGGVGCGRFALD |
| | NLEVYRWDLGTFAFRQAYSENNGLRSPEEEFDLAVIHELAEGLAKEDGQKILKGTEPF |
| | TCWQERSWQFSFTGPLLQGDPLAALNSDTADIISFRRTVVDNGEVLREPVLRGEGLRG |
| | LLRTAVGRVAGDDLLTRSHQDCKCEICQLFGSEHRAGILRFEDLPPVSPTTVADKRLD |
| | HVAIDRFDQSVVEKYDDRPLVGSPKQPLVFKGCFWVQTSGMTHQLTELLAQAWRDI |
| | AAGHYPVGGKGGIGYGWINSLVVDGEKITCRPDGDSISLTTVTGDIPPRPALTPPAGAI |
| | YYPHYFLPPNPEHKPKRSDKIIGHHTFATDPDSFTGRITCKLEVVTPLIVPDTEGEQPKD |
| | QHKNFPFFKINDEIMLPGAPLWAAVSQVYEALTNSCFRVMKQKRFLSWRMEAEDYK |
| | DFYPGRVLDGGKQIKKMGDKAIRMPLYDDSTATGSIKDDQLISDCCPKSDEKLQKAL |
| | ATNQKIALAAKHNQEYLAQLSPDEREEALQGLKKVSFWTESLANNEAPPFLIAKLGEE |
| | RGKPKRAGYLKITGPNNANIANTNNPDDGGYIPSWKDQFDYSFRLLGPPRCLPNTKG |
| | NREYPRPGFTCVIDGKEYSLTKRCERIFEDISGGENQVVRAVTERVREQYREILASYRA |
| | NAAGIAEGFRTRMYDTEELRENDLVYFKTAKQADGKERVVAISPVCISREADDRPLG |
| | KRLPAGFQPCSHVCLEDCNTCSAKNCPVPLYREGWPVNGLCPACRLFGAQMYKGRV |
| | NFGFARLPDDKQPETKTLTLPLLERPRPTWVLPKSVKGSNTEDATIPGRKFYLRHDGW |
| | RIVMAGTNPITGESIEKTANNATVEAIMPGATFTFDIVCENLDQQELGLLLYSLELEEG |
| | MSHTLGRGKPLGFGNVRIKVEKIEKRLSDGSRREMIPPKGAGLFMTDKVQDALRGLT |
| | EGGDWHQRPHISGLRRLLTRYPEIKARYPKLSQGEDKEPGYIELKSQKDENGVPIYNP |
| | NRELRVSENGPLPWFLLAKK |
| SEQ ID NO: 22 | MSNQTRWIIEGTLELITPLHIGTGLDEKERDENKETRWLEAVALDHKGQPYIPGASLK |
| OBEQ011807420 | GALRALAKRHDFRNLFDNKEVDGDFVRQAEFLSAWCVPDTDKGRLIQPRVAIDRVT |
| | GTAQDKKLFQTRLITPGTRFAMKIVVQNAVENEIADLLGLLNLLPDDPQFSLGAYANQ |
| | GQGRVQWFGKIQTRCFGINEAKAWYEEIRKDESKCWTAFAKPKNVSTPPTPAKEAQL |
| | TLPLNLAFHTPFLVKQAGIKADDADAVPRRTHDDKIVLPASSLRGRLRTQSERILRTLG |
| | CETPQGHTAPAYRKGQPHDDLAVLLFGAAGWRGIVQTSDCIVEDKSIKTRRHEMLAI |
| | DRFTGGGKDGAKFNVDYVECPTLAGKLSLDLARLKNAKLKGGKDALLPALGLMTL |
| | MLRDLAEGDIPFGYGISKGYGQCRASSALGDWAELLKQHLGADSADTTVQALREYL |
| | GNPKGQELKLDPPSADATQAGVPAQQNAAKTQAQGAQEKFHNPYHFIPLSKPDISQW |
| | PEPQKLTEKGHSHDRYASLSGRIVCRLTTQTPLFIGSEQTTPTNPQAPKSLHPFKLNNG |
| | LAIPATSLRGMISSLFESVSNSNFRVLDEKTYSMRKTMQQSLSAMGRIVRHDQKLYLL |
| | PLTLPTLPQGPHGVYDLGEKWSAVFDWQPPPLRIYFDPPPRRTYQSQQPCYMKLSTV |
| | KYSESNPNQIIAGENLGALRFPRGNQNTQFLIGQSNQDECPITQAEYAQKSEDERNEYT |
| | SGWVRTLVKPGRDLPRSVKHHVFLPDVFIDAPPPVNDLYPIPDSVIQRFHDLADQVLA |
| | SMNLKPEEIVDSTNLLPYTPVGRRSDSDCRDTRLQAGDLIFFDIDPPLHPGEKSQITEISF |
| | SSIWRSGIGKDHLLTTPDLLTNFDVNLQPHGMPGRTQSLSPAELLFGLVGTQNDQATT |
| | AYAGKVRIGFGLPEEGHNPRLDARITLKELSSPKPPSPALYFRKKSGKDEYVSKANLA |
| | DKPEDYILRGRKMYLHAWRKNEQVVELSDTGHDGGVRPPWVSKFDESADEGNKRR |
| | VSIEPIAKDESFYFEVDFHNLSRTELAQLCATLYPNEKFEHRLGMGKPLGLGSIKITPLS |
| | LFLVNRSQRYATDGLDKPRYHAVWHTGTASEPRWPDHLQREQQGIAFEGVSTAPTV |
| | MSLAAEAKVSDDVKRALELLGNPDEITVPVHYPQLHNGLMESKQFDWFVQNDKSGR |
| | DQPANNRQHLSSFTKDTEKLEPLIRIMRR |
| SEQ ID NO: 23 | MTTPSAPKSSLPALHWLIRAELEVLTPLHLGTGTDQRITPDAPDADPYWQADIALDAD |
| OVOO01000106 | GRPYLPGASLKGALKALARRRQVDAPCLPLFGDLNRGGAPHPDCIPPRRTRAGLAEFR |
| | DALQSHATQADGPDTAQPRIAIDRITGSVVDKKLFHTQTVPVGTRFSVEIILRRADQNL |
| | AAQLVALLQHGPTDPDFRLGAHANLGFGRVGLYGNIDTRRFGPQQAQHWFAAAQTQ |
| | ADARWTDFAEAVTLTAPAPAPAQPAPHRLALPLSLTFHTPFLVKQPEHKHRKPQDNA |
| | PDGTPRQRGDRALLPGASLRGRLRSQAERILRTLGCKVAQGHAVPPVKNNTCPDPAT |
| | LLFGTAGWRGLLRTDDCTGTAPATLVDHDMLAIDRFTGGGKDGAKFKLRYAECPTL |
| | EGQLSLDLSRLRSARLDGANAADTPWIALGLLTLVLRDLAEGDIPFGHGSAKGYGRC |
| | RAQGLPDRWRQALEAHFGPNADARALAALRAWCRTHATAALDAPCSLAGSAPTPA |
| | AAAPSGQAAPADAFHNPYHFIPFSQPDIDRWLSPDAHRKTGGHSRYRGLSGRLVCAL |
| | TTVTPLFVGAAARTPASDQHPKPVAGFALQNQPAIPATSLRGLLSSLFESISGSNLRVL |
| | HPTPYSIRKTTKEALSAIGRIVERNGELKLYPLTLPTIHQNADNAYPVPARWRKVFYW |
| | ESPVPLRVYFGSRKQTYDSRQPHYLPIQELSYLPNDSDCIAPDQGDLRFPSRDRDRKFL |
| | IGQCPISRYDCPIPETDLPKLSPQERPRYTRGWVRSLWTSNREKELPHTVKHQLFIPDP |
| | VETPAADDLLPIPQGVLDTFHALADLALAGQHWGKDETPADDQLLPFTPAGRQRHD |
| | ADRPPRDADRQTRLQPGDLVCFDLGDDGAVSEISFSSIWREGLRLAGKPNLATTADLL |
| | AQVSPHLLPLGMPGRSARLSPVEQLFGVVEYRPPQTAKGTRKPTDAPAAYALAGKLQ |
| | VGFGRPARPFEREPAVTLKELSTPKPPSPALYFRPKAGDGYVSKAKLASQPQDYAPAG |
| | RKHYLHALRRQGQVARLDNSGHVPSDGSGRPPWQSRFDGQEDSGNKRRVRVEPIPA |
| | GETFHFEIDFDNLSPTELEQLCATLLPHPAFEHRLGMGKPIGLGSVKLAVEGLLLVDRP |
| | RRYAEDEPNAPRHHRGWRANADAGWPDHLQGDSPAAPLEATEQPAALAERAMARV |
| | PADVRRALQLLGNPGAVAAPVHYPQVKDAQIEEKHYLWFVANDDEKTAGGNRHLP |
| | RLHANSPGLPTLPRLVKREKDHSSNTGKPRRK |
| SEQ ID NO: 24 | MIPDLRSLVVHISFLTPYRQAPWFPPEKRRNNNRDWLRMQSYARWHKVAPEEGHPFI |
| PDWI01005922.1 | TGTLLRSRVIRAVEEELCLANGIWRGVACCPGEFNSQAKKKPKHLRRRTTLQWYPEG |
| | AKSCSKQDGRENACPFCLLLDRFGGEKSEEGRKKNNDYDVHFSNLNPFYPGSSPKVW |
| | SGPEEIGRLRTLNRIDRLTTKAQDFFRIYEVDQVRDFFGTITLAGDLPRKVDVEFLLRR |
| | GLGFVSTLCGAQCEIKVVDLKKKQNNKEDSILPVSEVPFFLEPEVLAKMCQDVFPSGK |
| | LRMLADVILRLREEGPDNLTLPMGSQGLGGRLPHHLWDVPLVSKDRETQTLRSCLEK |
| | IAAQCKSEQTQFRLFCQKLGSSLFRINKGVYLAPNSKISPEPCLDPSKTIRTKGPVPGKQ |
| | KHRFSLLPPFEWIITGTLKAQTPFFIPDEQGSHDHTSRKILLTRDFYYRLPRSLLRGIIRR |
| | DLHEATDKGGCRVELAPDVPCTCQVCRLLGRMLLADTTSTTKVAPDMRHRVGVDRS |
| | CGIVRDGALFDTEYGIEGVCFPLEIRYRGNKDLEGPIRQLLSWWQQGLLFLGGDFGIG |
| | KGRFRLENMKIHRWDLRDESARADYVQKCGLRRGVGDDTAINLEKDLSLNLPESGY |
| | PWKKHAWKLSFQVPLLTADPIMAQTRHEEDSVYFQKRIFTSDGRVVLVPALRGEGLR |
| | GLLRTAVSRAYGISLINDEHEDCDCPLCKIFGNEHHAGMLRFDDMVPVGTWNDKKID |
| | HVSCSRFDASVVNKFDDRSLVGSPDSPLHFEGTFWLHRDFQNDVEIKTALQDFADGL |
| | YSIGGKGGIGYGWLFDMEIPRSLRKLNSGFREASSIQDALLDSAKEIPLSAPLTFTPVKG |
| | AVYNPYYYLPFPAEKPERCLVPPSHARLQSDRYTGCLTCELETVSPLLLPDTCREKDG |
| | NYKEYPSFRLNNTPMIPGAGLRAAVSQVYEVLTNSCIRIMDQGQTLSWRMSTSEHKD |
| | YQPGKITDNGRKIQPMGKQAIRLPLYDEVIHHVSTPGDTDDLEKLKAIVLELTRPWKE |
| | LPEEQKKKRFEKCKNILDGRMLQQKELRALENSGFAYWRDKTSLTFDSFLKDAIEQE |
| | YPRYSGDYQRIKALVVNITLPWKLLKKEERHKRFDKCRRILKGQQPLTKDERKALEES |
| | GFANWHGRELLFDRFLKDENSCLIKAETTDRVIASVAKNNRDYLFEIKQQDFARYKRI |
| | IQGLERVPFSLRSLAKSKETSFQIACLGLRRGRFLRKGYLKISGPNNANVEISGGSHSNS |
| | GYSDIWDDPLDFSFRLSGKSELRPNTQKTREYPRPSFTCTVDGKQYTVNKRCERVFED |
| | SAAPAIELPRMVREGYKGILTDYEQNAKHIPQGFQTRFSSYRELNDGDLVYYKTDSQG |
| | RVTDLAPVCLSRLADDRPLGKRLPEEYRPCAHVCLEECDPCTGKDCPVPIYREGYPAR |
| | GFCPACQLFGTQMYKGRVRFSFGVPVNSTRSPQLKYVTLPSQERPRPTWVLPESCKG |
| | KEKDVPGRKFYLRHDGWREMWGDDDKPDSRPSSEECQDIIEGIGPGEKFHFRVAFEN |
| | LDKNELGRLLYSLELDAGMNHHLGRGKAFGFGQVKIRVTKLERRLEPGQWRSEKICT |
| | DLPVTSSELVISSLKKVEERRKLLRLVMTPYKGLTACYPGLERENGRPGYTDLKMLAT |
| | YDPYRELVVQIGSNQPLRPWYEPGKSFKPSPGNDCTGRGGSVSKSLISEPKVVPAIAPF |
| | CEGVVKWFNSVKGFGFIETKEQRDIFVHFSAIRGEGYKILEPGEKVRFEIGEGRKGPQA |
| | INVIRIR |
| SEQ ID NO: 25 | MKMNKTWPFREHWEISGYLRTVTPLHIGSGRTVTRPELTVADRDELVDINAVVTDYT |
| VAPF01001339.1 | GKPYLPGSTIKGALHAWLQKRLKEESRTCLIQLFGQEEEAEEKKKNNHGGKAEFFDA |
| | RVIFPHTGPGSLPYWDDCRQTYVAATVAIDNITRTARHRHLIHAEMVPPGVTFALTLA |
| | GPLDEEDIGLLLAALQGFNESPPALVIGAHTANGLGRFSWELSTVRRFGKEHLQGWLE |
| | AETRAMRTEAMQPLSREGVEDLLQDGIAQIDHNEDQVRLGLELCFDGPFLVNDPPTK |
| | KEQDDKKKRRSNTPNLRPLRDAIGRPCLPESSVRGALRAQAERIIRTMEGTCSEDNPA |
| | YKKEIHTDAEIEELSAVCRVFGAPGWKSLLEISDFEFVDGEDCDNIQEFVAIDRFTGGA |
| | KDKAKFNAEYIGSPRFTGTIALDKRRDLPDWGKGLLYLVLRDLAEGDITLGFGRSKG |
| | YGVCRAKIKNLDLLLPEKSVAALHKKFAISPADDKPATDQIAEDTTSGNLGISAGGPA |
| | QKTTESYEPPNPSGPGTFHNPYHFVPVVKPSAADRQHWLDKGILPSASENKTQHTHA |
| | CYLDTTNGKKIYHGRIVCRLQAETPMFIGGRHRENTEPTEILPFTLGGKPAIPATSLRG |
| | MLSSIAEAASNSSLRVLEDKTLSYRKSMRANRNEDKPLSALGMIKKIETGDKVEYRLL |
| | PLTLPTLVKRGQYYILPEEYQTMFPDGRAKLKVYLNSNYTASDGTNQDFLKGKKSWR |
| | LPHGEIYYMKLCQDFSLQNGQLTFDSQNQNMLHFPKNRNNFVVGQRSIDNTPPMTKA |
| | EWRTNHQTGVPGMLRIMQASGRNFPTGRYHEIFIPVPTKKDCKQLYPVDEKAVERFL |
| | DLAGEQTKSQQNEKNLKQYQILPYHPVGTKRNTDPETNDRYMDLNSGDIVYFRPDAT |
| | GTKVEEISFSSIWRDRVEDDNHNRAGVHAFFGNIDKELLPFNPKRAEISPAELLFGFVE |
| | ERERGKVDDGQAPAFAGKVRVSFGRLSSEKKPDTIFQDQVTLKALSSPKPPSPALYFT |
| | GNGNGSIAKPDLTLSRHSPQGRKFYLHAWQEENEIIKFLSNGKKTSPTVINGLYPWES |
| | KSNLSRQLPDKHAKLKSAITPIKKGTVFYFHVDFDNLSEWELGLLCYALQPGKEFRHK |
| | LGMGKPIGLGSIHIEPAGLYLVHRGNRYSLDGVPDNSRYNGGIWQSEDKRLQEWQEL |
| | YPRESTAAASSAAASPADFALKFSGTMTPSVQQALKRLGDPDNVVAPVHYPQVEGA |
| | DLEEETYKWFVANDVGSQTKVGNRTTCTEEAARKSMLPLAGRGPLPRLKRYKWCP |
| SEQ ID NO: 26 | MAAVQDRWTLMDQQGNELKRFRITAELETASSLHIGASETVEHDLIKNDDGTPVQIN |
| DRKI01000155.1 | ALITGAGGLPIIPGSTIKGRFLARLRERGVDSALLETLFGKGHDRETEDQGRGGRAEFH |
| | DAPLCHRLSGARHFPYWRPERQIWVKAQTAVDRHRGTALRRSLRYTEMVPPGVRFR |
| | LTITGCMTDAEADVLFALLEDLGDPRQACSFGGAGADGNGTMRLFGRPEVYCLDRS |
| | GILGWLASFEKGGNGGMAMTAAALLQADTVQRRADKVRQAWQPPDVGPRLHVELR |
| | FSGPFLVNDPSRNTPDITQAPDMVPLVDEDGNPMLPASSFRGALRAQAERIIRTLGGRC |
| | CDTSSPCRPLGSSDKVGELCLACQVFGAPGWGTTLHIQGFTCTSVFRREQEQTFVAID |
| | RFHGGCKEGALYTIRHAESPRFEGHLVIDPRMPAWGRGLLALVFRDLREGDITFGLGA |
| | GKGYGVVDAAVVQDMAELEPYVEAFREQCRQHQGMADCHSAPSPQPLRDHDLAEIP |
| | PAEEAPGETFLNPYHFVPIREPDTGSWLARDELDSSCCHSHGFYRQQVDDRPLYHGRL |
| | TCLLETETPLFIGATGDSSVPSRIENYRLGNRIAIPAASLRGMLSSLAEAASNSAMRVL |
| | HQGILSYRKKAKNALREIGMIVLRDGKRFILPLVPLMEVTKLRHAYTDPAMKHFLDD |
| | KNSWSPRCNRVYYLGRDGNQIPAETRGAGMRPGILRLLGREGRHDALQNKKHEYFIE |
| | IPERYVDQDHCFDYRMFIRDRARNGTLVPISPVAWERYHCLAEERTLSQKNDPELRED |
| | KACASLKWLPFHPKGRVRERDPENDVCHLSLRHGDLVFYAEQNRVVSEISFSAIWRS |
| | RVETSDSYQAVTVDCFVPKELRPFNRDRRAISPAELLFGFVELDESEHSTEKSRYEQM |
| | AFAGKVRLSAGLPVEDVEDSALLEPKPIVLKALSSPKPPSPPFYFVMRDGSGAYIAKK |
| | DLSPDRHRIKGRKHYLHGLRQRGNPDRVQSLDRYGHATETAANPPWETCHPEERPQI |
| | KVRVQPVRRKTKFFFHLDFSNLSRWELGLLCYVLRPTACFRHKLGMGKPLGLGSVRI |
| | DIASLQLIDRVRRYGTDDLTAGRYNMGGHFNASCLDLLPQQDSPAPDDSGAAPDPGT |
| | LRQDFVKTMDETVFRALDLLGNPAHVQRPVHYPQVREMDIEDQTFLWFVDNDKQW |
| | KDALQPLTSSSTQLPPLTRRNKR |
| SEQ ID NO: 27 | MTTVKEKSWAFTGLKRWKIITTLETQTPLHIGSGEVAEIEINDSQGDRRQVQANAIIRG |
| DRNY01000543.1 | KDDKPIIPGSTLKGKLRSHFESCLDHSKALERVFGKEYQSDEEQGRGGLAEFHDAVCS |
| | YVAPGNSYYPNWNEARNTYIEASTTIDCHTGTAADATLHYNECVPPGTRFLVTVTGA |
| | MSDKDAALIVAALQAFGDETNPIHLGAEEANGKGRMGLFGNVEVSCLDHDDIIAWIS |
| | QGSDARMATDKFKPLGKEKVNDLAKNITTPTATGGAQRQHFGIELKFDGPFLVNDPS |
| | KYSKGDGDQPAVHQPLTDRSNNPILPARSFRGAIRAQAERIIRTMGGACCDTQSPCQN |
| | SGQLCIACQMFGTTGWKTTLSISDFTYDGEYRPAKTQQFVAIDRFHGGGKDGALFSIK |
| | YFERPVLKGGISLKLRNQNADELSWRKGLLALLFRDLQEGDITFGFGANKGYGGVEE |
| | ACITNADVISTADIEAFRAKCHANHADSWCSPVSKPTNRDDKSSLPSINPATGAGHAF |
| | HNPYHFIPIKAPDTSTWLDKHKLATPGSPHSHAYYRSCSDDDKPLHHGRITCKLTAET |
| | PLFVGSGDAENQLTDSEAKLKEHYQLNNKLAIPATSLRGLISSLAEAASNSALRVLDN |
| | GVLSYRKPASRALRKIGILFKREEQWRLVQMEGNLANAIKLKSAYTNQKMMDFLAN |
| | KQSWSPEHNVVYYLSADFRPGDVPQETYLAGRICGILRILGGKDGDRKNELENKKHE |
| | LFIRVDEQYVDTEINRFDYEEYVRQGGIPVSPSAVERYTELADQRSLSQKNSRDLKGD |
| | NNCCSDKWLPFHLKGAARYKKEKACLLPLREYDLVYFDSDGTQVTEISFSAIWRDRV |
| | ADKVHAFFPEELRPFNQKRKWISPAELLFGFVELNDNKDERDHAQAFTGKVRVAAG |
| | VLSPDDSIRQGDLQEHEPIMLKALSSPKLPSPALYFKQKSGDHRYIAKPDLKKASHQA |
| | QGRKIYLHALRDQKDDVQKLNTKGQPANGNGAHLPWKTADEDERPQLKVRIRPLKP |
| | GTSFYFHLDYNNLTEWELGLLCYVLQPSETFRHKLGMGKPIGLGTVKIEIATLQTIDR |
| | QKRYREAGANEHRHNGSNWVNESLRDELERLPGTVELSPDRQPEAKLRPDELRQSFI |
| | ATMDNDIYRAIELLGDPHNIKYPVHYPQVRNKSIEQENFKWFVANDSGSGDQRKGTG |
| | IDAKEEPMRSIDQISTTIPTLNRYEWNGD |
| SEQ ID NO: 28 | MARNNKQYHFIPRWEIKVNLTTRSFLHIGCDEFTDRPGLEIEQKDGSKVKAEINAFIKD |
| DTXS01000070.1 | SNGKPYLPGSTIKGNIRKWLETNKKADEETCKLFNTLLGFTVKMQDEGCGGSAEFHN |
| | AVISSPLEDGNNFPYWDVDLQTSVETSTVIDRVTGTVVDGRLFSTEVVPPEVSFTLIITG |
| | AMTEQQVSLLTAVIKDGFAEDCPTPITIGADSGNGFGRFRFDSIHMKCLGTGEVLNWL |
| | EDGSQDMAATAMRSLSPDDIEQHIIKGRNYLKSPSVSDTVTIEFGFAGPFLVNDPSRKK |
| | RKEDIDHQPLRDSAGNARLPAKSIRGAMRSQAEKIIRTLGGWCCDPVNPCPSVFSVVEI |
| | NDRLCLACRVFGATGWKSRISIQKVEYKGTAESTRQETVQDFVAIDRFHGGGKETAK |
| | FDASFSWRPQYSILMHIPSDLEGWAKGLLALTFRDFKEGDIFLGYGRSKGYGRVDSDS |
| | VKPGIDTMLTESNLELFRRKCDDNPGEYPCKTRQPPNLVQPVERNNLTEAADEGSFH |
| | NPYHFIPTPKPMIESWLAKEDFDETMHDSHALYRDVDENEEPLYHGKISCTLTTETPV |
| | FVGGKHDPRNDTEPQQVDHYTENGEIAIPATTLRGLLSSLSEAASNSSMRVLDDGMM |
| | SYRQPVGSGSLSAIGMVVIRDGKKFIYPLALPIFGERDKLPQEYHIMFPYTQKAPLKVY |
| | LERAYLAGNMKSFLDKQNSWNLLNEKIFYLPVPEFSFSRVHTMGAENRDVLKISRRG |
| | NLILGARLPVNLCPRSKEKALPGDIPGILRILGKEGRDGEVPVGKKHELFIPVSDGFASN |
| | PRSFIDNLTSKELFKIPDEVVDRFEELADDRTTQQIKHPGNVKNNNQWLPFHLKGCTR |
| | NDGLTGKDEKRLRVQEGDLMYFRPSPQSPQVAEISFSAVWRGRVNKTVHNYFPPELV |
| | QFNKNREKISPAELLFGFVQQDKHEKSLSFAGKVVLSSGKQLRETESVSRENEVTLKIL |
| | ASPKLPSPSLYFKRENYIEGGNYIAKNEMNNSSNIKPRGRKQYLHALSNSEDPKGVQK |
| | ISRTGSVDDGGNYPWQSMNNDNIKQKVCIRPVSKDGCFTFEMEFENCTEWELGMLLY |
| | ALRPSQQYRHKIGMGKSIGLGTVRIDINNLQFIHRKNRYNAGIIDVPRYNYEAGHDMD |
| | YFHNKFADTIMPEIKNSIELLGDPRNVRFPVHYPQVHGADIEDKTYQWFVANDSGTN |
| | NGQNGAAYKKNKAEESSLTELDEISNTIPGLERHEWLGR |
| SEQ ID NO: 29 | MALKTWTLNGEERWHISVVLETVTPLHIGSGEFCYRPELTNADQKPVDINACIKGAN |
| JABFST010000317.1 | NLPIIPGSTVKGKFNAWLTARQVDTPLLEAIFGKGHNPDDDDQGSGGKVEFHDAWIST |
| | KIKDTSTWPYWQVATQTFIDAATAIDRHSRTALDASLHYTECVPPGVQFTLNITGVM |
| | QEHEAALIIAALDRFDQHDDQPYFGAGDANGQGQLILVGHLAVKVMGKTEITEWLA |
| | HFNNKASDMAMSHARSLGAEDIAGLIKLGQTLLKPVPPTVSLGIQLQFAGPFLVNDPY |
| | AVKKLEADPKTKIDHYPLLDNHKKPRLPSASIRGVLRSQAERIIRSLGVHCCDTRDPCP |
| | SLYKHQDLSQLCLACQIFGAAGWKSVINISDFTCVDANELKTQEFIAIDRFHGGGKDG |
| | AKFNAKHSERPYFQGRITLSPRMANHQLDWGKGLLALVIRDLQEGDLSFGFGANKGY |
| | GALESVLITGIDQLQTDAIEAFRRLCVTQAAPQAFITPTSAVVIGDKAPLVVTDKKLPD |
| | NSFHNPYHFIPINSPDTRHWLPTETDLAESHHSHAYYRQQPELFHGQLICRLYTETPTFI |
| | GASKKDDTLPAELDNYRLNGQLAIPATSLRGMISSLAEAASNSAMRVLDNGLLSYRK |
| | DASLALSKIGITFINRQGQWQLIPMEKIKLKNAYSAENMRLFVEQSHSWSPDYNTVYY |
| | FSEKAGAFDVPQRTPKPGWQPGILRLLGKEGRSQELENKKHEWFIPVPENYIDKQLNA |
| | FKYQEYLKDNSSKAIDIPAPVLNRYNELAYQRTLSQKKDTELVADGDSPAWLPFHLK |
| | GQQRQPQMVGKHLVYTLPMTEYSLVYYAATNKVATEISYSSIWRGRVQDDADQAAT |
| | VNHFIPDDLLPFNPKRTSLSPAELLFGFTELDPDKHSNDPTRSFAGKVRIGAATLAAYP |
| | SNDSDLLAPEHITLKALSSPKLPSPALYFRTLQGNNSNVYIPKHELNPNHHTAKGRKY |
| | YLHATRTPDQKRILKLSDQGHPPQNNAVKLPWLSHQETKNLQLKVKIKPIKPKQSFYF |
| | QVDFNNLTAWELGLLCYALRPTIDFRHRIGMGKPLGLGSVKIDILALQTLDRQKRYA |
| | QDSQDSARYNQHRWVNSSVTDMLAQAGYDVIEPTANPLVPKDLKTLFSQTMAANID |
| | RALTLLGEPQHVKQPVHYPQVRDTAIQVRDTAIEEESYQWFVANDNLSDNSSAAKQT |
| | LHDITETSEGLPTLIRHQKKKETQP |
| SEQ ID NO: 30 | MSDTQKQAIHENKWHFRGIKRWEISAYLKTLSPLHIGDGGTIPVTIKDTQGKNREVEV |
| PDPY01000001.1 | NSVITGKAALPIIPGSTIKGRLRHYFSKHFSDKALLNKVFGEESDATDDDQGRGGLAEF |
| | HDAKWNPEKNRNLQGRYPYWNNTRKTYIEVSTAINRHTGAAKDKSLHHTECVPPGT |
| | VFEIKITGSMDDRCAALVVAALEAIQTTGSRIFLGAEDANGNGRIGLTGKITVKQMDQ |
| | AHIIQWLQKDSTTCVASFSNVKAENETQVKQMVQRHIAPKLNSVVSAAGPSYDITLHF |
| | DGPFVVNDSDKCKAEDTPDIYPLEEKNGVPAFPVRSFRGAIRSQAERIIRTIGGQCCDG |
| | SINNTCKNPKNLCIACEMFGSTGWKTSIEMDPFLCVDRELKPFIIQEFVAIDRFHGGGK |
| | DEAKFNAAHYQAPVFKGKVRVSQRVGNDISWRKGLLALIFRDLKEGDIYFGFGTNKG |
| | YGAVKKAEINPDGNASDFSESDIEAFINKCREKKGLYNCNPIKKPGKTKVSKNLPPAIV |
| | PLDRTDSKFYNPYHFIPVKKPNTSSWAEKTAFGTADSPHSHGFYRKQTNEQQPLYSGR |
| | LICMLTSETPFFIGAQAESDPTENENQASLRHPYQLDGEPAIPSTSLRGLISTMTEAAAN |
| | CAMRVLDSEIISYRKPMNPSHILSALGMVTKRGEDFWLIPLAMPALSLNDEEHNYKLD |
| | KRYRTMFPDGLAKLKVYLEKAYSNNVMKTFLNNENTWTLAQSKIHYLPLTPIQMQN |
| | GGINSYYNNLRTPSRSNNFLIGQTVAHGNGIPASGPGAGMVPGILRILGKEHRQNDLP |
| | QNKKHELFIPVPDAFVADPKTFLDTATAFLIPRNVIDAFEKIAEKQTQSQKQDKLKHD |
| | EERLPFHLKGTRREQNHTLQIKTGDLVYFRPNAKGDEVEEIAFSSIWRGKTSGTTADFF |
| | PDKELLPFNRNRSRVSPAELLFGFTENNPKEMKIDRGLAFAGKIRISAGTLSDKFSDTT |
| | ESDLFEPETTLKALSSPKPPSPALYFKEKKSGTQYIKKQDLNPGKHEIQGRKIYLHALR |
| | NENNQNVQRITSQGKFDNAANRTQPWVSQNEERNHLKTKCKPLKSGLNFFFHIDENN |
| | LTQWELGLLCYALRPCETFRHKIGMGKPIGLGTVKIDIAVLQTIDRYARYTDTTQDSE |
| | RYNQGAWISQELQNEIPNQYKGKGISNKKGMLSPEDCRKVEMETMDADIQRAIELLG |
| | DPGNVTSPVHYPQLDRKNIETKNYEWFKQNEIEQQVLKPITKNTTHLTPFARWEQG |
| SEQ ID NO: 31 | MNLPTWKLNNEKRWHISIVLTTATPLHIGSGEFCEHDDVKNNDGEPVKINACIKGSKG |
| NZ_JMLA01000001.1 | RPIIPGSTIKGKLYEWLKTRNTEENLLEKLFGKGHNSVSQDQGRGGKAEFHDAEIIEPL |
| | TGSQPWPYWREEHQAFIAASTAIDRHKQVALQQSLHYMETVPAGIRFKFTFTGVMRD |
| | EEAALLIAALDSFDKNQNQPCFGVDRANAYGRMELHGHLHVKVMGATEISSWLNSF |
| | SENDKKMAMESARNLEQQEINTLIKQGNALFKASCDEVKLGLTLKFKGPFLVNDPYA |
| | VKILSSNENAKTDHYPLLDKNRNPYLPVSSFRGVLRSQAERIIRTLGGKCCSTDDPCKP |
| | IFDKGDLSKLCLACQIFGASGWKTVINIHDFKAINKSKKTKQDFVAIDRFHGGGKDGA |
| | KFDATHFERPEFEGAISFSPRMANNDLDWGKGLLALVLRDMQEGDMTFGYGANKGY |
| | GGLESASITGIEQITSDIQAFRDKCVASPQTWLCDEAVKPANQQDKIPPAGIQVANSGF |
| | HNPYQFIPSKDPDTGHWLPVLGLNADSHHSHAFYRDQTDNGEKLYHGRLICCLNTET |
| | PIFIGADKKKDTEPAEINNYRLNGELAIPATSLRGMISSLAEAASNSAMRVLDNGLLSY |
| | RKTADDALRKVGMVIYVDNKSFIIKLNDAIKLKQTYTPGNMKDFIEKSNSWSPEHNT |
| | VYYLDNNQIPQESYMNGMKPGILRILGKEGREQELENKLHELFIPVPLEYVDTENNKF |
| | DYQAYKKAFLYRAIEIPEPVLKRYSELADQRTMSQKSNKELKKDDTCQSVGWLPFHL |
| | KGTKRQLDDKHKVGKLQIDEYDLIYYEASGKEVTEVAFSSIWRGRVETNSSQANKVY |
| | SFIPGELLPFNESRKKVSPAELLFGFTQINKDGSKADDKAQAFAGKVRISAGTISEYPES |
| | EANLLEQEVTLKALSTPKLPSPALYFRTINGNGSAYISKQELEPSKHLAKGRKYYLHA |
| | LRTGDNKVQKLGSQGETANGGDSKLPWVTHNPDERPQLKVKIKPIKAEFIFSLDENNL |
| | TEWELGLLCYALRPTDSFRHRIGMGKPLGLGSVKIDIMALQTINRQQRYAQDGLEEN |
| | RFNRHNWVNPPHQPRLDKAGYSISLSSTPLNPEILRATFTKTMNADIYRTLELLGNPQ |
| | NVKRPVHYPQVENHNIEQENYKWFVANDQGSGKGRNKIDPAEKALKILTENSDCLPT |
| | LSRLDWRDE |
| SEQ ID NO: 32 | MNNKGSNMTDTVKSGRWIITGQFQLVTPMHIGTGLDEEMDKQSGESVDKKQNNSWI |
| NZ_FOGH01000010.1 | QAIALDLNKKPYIPGASIKGALKALARRYYCASNLNIFGDTIDTKDGDNKRKSVTVAG |
| | QAEFLNAWYAADQEDKPFDTITRVAIDRVTGTAEDRKLFNTRRVNPGVCFNYKIIIQN |
| | ACETEIQYLLDLLRKAAKDPSFSLGAGANQSQGKVRCLSSCVRYFGKQEMHDWFRAI |
| | QNGKQEHWQIFAKPSNIKYADLERPDIIANSLNLPLTLDFHTPFLVKASKKKDEAKNE |
| | ADAKPRTNHQGQVILPASSLRGRLRAQAEKILRTMGQDIPQGHAAPAYDGIAHRDLIS |
| | LLFGTAGWKGIVCASDLIHSIPEYALQFNGVRETISDLSDTVKSCIIVDLVKTSTAAEKT |
| | EEQLHIRIVDSAGSLIVHKSENSSWANDTFRDASVKDNFKARLKEIADPQDLSDALRA |
| | DIKKRAFQLATLTRHEMVAIDRFTGGGKEGAKFNVDYIECPTLTGAIYLDLHRLKQA |
| | QLKNDEDALKPALGLISLLLRDLAEGDIAFGFGANKGYGQCREHAVLDNWEERLKKI |
| | GAGLTIDGALQALRDTVALEPPAEFPPEIEKTTDDNQPEAPDFNLKPASNGFHNPYHFI |
| | PLNNPKIGDWPEAKAETLKANREGHDQYHTGKFSGRIVCSLTTQTPLFIGAETKPSTS |
| | DREPSEARPFKLNGKHAIPATSLRGMLSSLFESVSNSNFRVLHPEHYSVRKSLDDYVA |
| | LSAMGRIVDDQGELKLQPLTLPTLFGNRNNVPAKWEKIFGTPSEDDFLRIYFDDIPSKF |
| | SSNKRYFYNCKATELKDFIKSDKYFIGKRTPTVFPKSSTEKSHLESLEFIDVEKFKKAV |
| | ENLEITPGNNPYIHGWVRNLKDEFREDIPDNVKHHVFLPDTTKRVSPLEIPPHVKKRFH |
| | ELADLALAGLHLKQGETIASPYKILPYTPIGRNKLENHIHRVPNDLTCYMTRLKKGDL |
| | VFFDVDNDGQITEISFSSIWRAGIGTKNKLQTTADLLSQRDPNLVQLGMGVRTKNTDR |
| | FKLSPAERLFGVVEHRDDDNTTVENVNQPNDKAQAFAFAGKVRIGFGLPDKKTTVN |
| | GVSPVTLKELSSPKPPSPAFYLKRKNNDDFVSKKVAAECSETMTLRGRKCYLHAWRE |
| | QNGNVMKLDAIGVNSGGSTCKPPWKTHKPAANDQKEFEEDKNKFITSRQVKIAPISE |
| | NTPFYFEIDFNNLDATELAQLCATLQPAPKFEHRLGMGKPLGLGSVKIEPVGLFLINRH |
| | QRYTTDSTNCDRYHYAWLKGEHAAWDWPEYFRQNVVTADCTQTFNDTFDKLVQN |
| | GLAGTDADIKHALQLLGDPQYIGVPVHYPIAGNSTLENKHFEWFGNNDKASVLRQKA |
| | QANSKNHHYQPKQQATPEEPQYLHTITKDSKQISLLKKNKIEDIENRDQQKHRYSNHR |
| | R |
| SEQ ID NO: 632 | MFPKGRQMRRQRLLGDAEYYGGTGREQPASIVISTDSDPDHKVYEWIITGQLKAETG |
| MVRP01000104.1 | FFFGTKAGAGGHTDLSILLGKDGHYRVPRSVFRGALRRDLRVAFGAGCRVEVGRERP |
| | CECPVCKVMRQITVMDTISSYREAPEIRQRIRLNPYTGTVDKGALFDMEVGPEGIEFPF |
| | VLRFRGSKSFPSELAAVIGSWTKGTAWLGGAAATGKGRFSLLGLSIHKWNLSTAEGR |
| | KSYLAAYGLRDAADKTVKRLSIDKGGKGDVGLPAGLERDALPSSVREPLWKKLVCT |
| | VDFSSPLLLADPIAALLGVEGDERIGFDNIAYEKRRYNGETNTTESIPAVKGETFRGIVR |
| | TALGKRHGNLTRDHEDCRCRLCAVFGKEQEAGKIRFEDLMPVGAWTRKHLDHVAID |
| | RFHGGAEENMKFDTYALAASPTNPLRMKGLIWVRSDLFETGHDGPTPPYVKDIIDAL |
| | ADVKRGLYPVGGKTGSGYGWIKDVTIDGLPQGLSLPPAEERVDGVNEVPPYNYSAPP |
| | DLPSAAEGEYFFPHVFIKPYDKVDRVSRLTGHDRFRQGRITGRITCTLKTLTPLIIPDSE |
| | GIQTDATGHKMCKFFSVAGKPMIPGSEIRGMISSVYEALTNSCFRVFDEEKYLTRRVQ |
| | PKKGAKSSELVPGIIVWGQNGGLAVQQVKNAYRVPLYDDPAVTSAIPTEAQKNKER |
| | WESVPSVNLQGALDWNLTTANIARDNRTFLNSRPEEKDAILSGTKPISFELEGTNPND |
| | MLVRLVPDGVDGAHSGYLKFTGLNMVLKANKKTSRKLAPSEEDVRTLAILHNDFDS |
| | RRDWRRPPNSQRYFPRSVLRFSLERSTYTIPKRCERVFEGTCGEPYSVPSDVERQYNSII |
| | DDISKNYGRISETYLTKTANRKLTVGDLVYFIADLDKNMATHILPVFISRISDEKPLGE |
| | LLPFSGKLIPCEGEPPTILKKMAPSLLTEAWRTLISTHLEGFCPACRLFGTTSYKGRIRF |
| | GFAEHTGTPKWLREELDWARPFLTLPIQERPRPTWSVPDDKSEVPGRKFYLHHHGGN |
| | RIVESNLRNRPEVNQTKNNSSVEPISAGNTFTFDVCFENLEAWELGLLLYCLELSPKLA |
| | HKLGRAKAFGFGSVKIHVERIEERTTDGAYQDVTAVKKNGWITTGHDKLREWFHRD |
| | DWEDVDHIRNLRTVLRFPDADQEHDVRYPELKANNGVSGYVELRDKMTASERQESL |
| | RTPWYRWFPQNGTGGSGRHEQAATSQEQDTAKDESVLSATQRRQAVIDVSDPDERL |
| | SGTVESFDRQKGDGYIGCGVRQFYVRLEDIRSRTALCEGQVVTFRARKEWEGHEAYD |
| | VEIDQ |

### Guide Molecules

The system may comprise a guide molecule. The guide molecule may comprise a guide sequence. In certain cases, the guide sequence may be linked to a direct repeat sequence. In some cases, the system may comprise a nucleotide sequence encoding the guide molecule. The guide molecule may form a complex with the dead Cas7-11 protein and directs the complex to bind the target RNA sequence at one or more codons encoding an amino acid that is post-translationally modified. The guide sequence may be capable of hybridizing with a target RNA sequence comprising an Adenine or Cytidine encoding said amino acid to form an RNA duplex, wherein said guide sequence comprises a non-pairing nucleotide at a position corresponding to said Adenine or Cytidine resulting in a mismatch in the RNA duplex formed. The guide sequence may comprise one or more mismatch corresponding to different adenosine sites in the target sequence. In certain cases, guide sequence may comprise multiple mismatches corresponding to different adenosine sites in the target sequence. In cases where two guide molecules are used, the guide sequence of each of the guide molecules may comprise a mismatch corresponding to a different adenosine sites in the target sequence.

In general, a CRISPR system is characterized by elements that promote the formation of a CRISPR complex at the site of a target sequence. In the context of formation of a CRISPR complex, “target sequence” refers to a sequence to which a guide sequence is designed to have complementarity, where hybridization between a target DNA sequence and a guide sequence promotes the formation of a CRISPR complex.

In certain embodiments, the target sequence should be associated with a PAM (protospacer adjacent motif) or PFS (protospacer flanking sequence or site); that is, a short sequence recognized by the CRISPR complex. Depending on the nature of the CRISPR-Cas protein, the target sequence should be selected such that its complementary sequence in the DNA duplex (also referred to herein as the non-target sequence) is upstream or downstream of the PAM. The precise sequence and length requirements for the PAM differ depending on the Cas7-11 protein used, but PAMs are typically 2-8 base pair sequences adjacent the protospacer (that is, the target sequence). Examples of the natural PAM sequences for different Cas7-11 orthologues are provided herein below and the skilled person will be able to identify further PAM sequences for use with a given Cas7-11 protein. In certain embodiments, the Cas7-11 protein has been modified to recognize a non-natural PAM, such as recognizing a PAM having a sequence or comprising a sequence YCN, YCV, AYV, TYV, RYN, RCN, TGYV, NTTN, TTN, TRTN, TYTV, TYCT, TYCN, TRTN, NTTN, TACT, TYCC, TRTC, TATV, NTTV, TTV, TSTG, TVTS, TYYS, TCYS, TBYS, TCYS, TNYS, TYYS, TNTN, TSTG, TTCC, TCCC, TATC, TGTG, TCTG, TYCV, or TCTC.

The terms “guide molecule” and “guide RNA” are used interchangeably herein to refer to RNA-based molecules that are capable of forming a complex with a CRISPR-Cas protein and comprises a guide sequence having sufficient complementarity with a target nucleic acid sequence to hybridize with the target nucleic acid sequence and direct sequence-specific binding of the complex to the target nucleic acid sequence. The guide molecule or guide RNA specifically encompasses RNA-based molecules having one or more chemically modifications (e.g., by chemical linking two ribonucleotides or by replacement of one or more ribonucleotides with one or more deoxyribonucleotides), as described herein.

As used herein, the term “guide sequence” in the context of a CRISPR-Cas system, comprises any polynucleotide sequence having sufficient complementarity with a target nucleic acid sequence to hybridize with the target nucleic acid sequence and direct sequence-specific binding of a nucleic acid-targeting complex to the target nucleic acid sequence. In the context of the present invention the target nucleic acid sequence or target sequence is the sequence comprising the target adenosine to be deaminated also referred to herein as the “target adenosine”. In some embodiments, except for the intended dA-C mismatch, the degree of complementarity, when optimally aligned using a suitable alignment algorithm, is about or more than about 50%, 60%, 75%, 80%, 85%, 90%, 95%, 97.5%, 99%, or more.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, CA), SOAP (available at soap.genomics.org.cn), and Maq (available at maq.sourceforge.net). The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay as described herein. Similarly, cleavage of a target nucleic acid sequence (or a sequence in the vicinity thereof) may be evaluated in a test tube by providing the target nucleic acid sequence, components of a nucleic acid-targeting complex, including the guide sequence to be tested and a control guide sequence different from the test guide sequence, and comparing binding or rate of cleavage at or in the vicinity of the target sequence between the test and control guide sequence reactions. Other assays are possible, and will occur to those skilled in the art. A guide sequence, and hence a nucleic acid-targeting guide RNA may be selected to target any target nucleic acid sequence.

In some embodiments, the guide molecule comprises a guide sequence that is designed to have at least one mismatch with the target sequence, such that an RNA duplex formed between the guide sequence and the target sequence comprises a non-pairing C in the guide sequence opposite to the target A for deamination on the target sequence. In some embodiments, aside from this A-C mismatch, the degree of complementarity, when optimally aligned using a suitable alignment algorithm, is about or more than about 50%, 60%, 75%, 80%, 85%, 90%, 95%, 97.5%, 99%, or more. In some cases, the distance between the non-pairing C and the 5′ end of the guide sequence is from about 10 to about 50, e.g., from about 10 to about 20, from about 15 to about 25, from about 20 to about 30, from about 25 to about 35, from about 30 to about 40, from about 35 to about 45, or from about 40 to about 50 nucleotides (nt) in length. In certain example. In some cases, the distance between the non-pairing C and the 3′ end of the guide sequence is from about 10 to about 50, e.g., from about 10 to about 20, from about 15 to about 25, from about 20 to about 30, from about 25 to about 35, from about 30 to about 40, from about 35 to about 45, or from about 40 to about 50 nucleotides (nt) in length. In one example, the distance between the non-pairing C and the 5′ end of said guide sequence is from about 20 to about 30 nucleotides.

In certain embodiments, the guide sequence or spacer length of the guide molecules is from 15 to 50 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27-30 nt, e.g., 27, 28, 29, or 30 nt, from 30-35 nt, e.g., 30, 31, 32, 33, 34, or 35 nt, or 35 nt or longer. In certain example embodiment, the guide sequence is 15, 16, 17, 18, 19, 20, 21, 22, 23, 24, 25, 26, 27, 28, 29, 30, 31, 32, 33, 34, 35, 36, 37, 38, 39 40, 41, 42, 43, 44, 45, 46, 47 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nt.

In some embodiments, the guide sequence has a length from about 10 to about 100, e.g., from about 20 to about 60, from about 20 to about 55, from about 20 to about 53, from about 25 to about 53, from about 29 to about 53, from about 20 to about 30, from about 25 to about 35, from about 30 to about 40, from about 35 to about 45, from about 40 to about 50, from about 45 to about 55, from about 50 to about 60, from about 55 to about 65, from about 60 to about 70, from about 70 to about 80, from about 80 to about 90, or from about 90 to about 100 nucleotides (nt) long that is capable of forming an RNA duplex with a target sequence. In certain example, the guide sequence has a length from about 20 to about 53 nt capable of forming said RNA duplex with said target sequence. In certain example, the guide sequence has a length from about 25 to about 53 nt capable of forming said RNA duplex with said target sequence. In certain example, the guide sequence has a length from about 29 to about 53 nt capable of forming said RNA duplex with said target sequence. In certain example, the guide sequence has a length from about 40 to about 50 nt capable of forming said RNA duplex with said target sequence. In some examples, the guide sequence comprises a non-pairing Cytosine at a position corresponding to said Adenine resulting in an A-C mismatch in the RNA duplex formed. The guide sequence is selected so as to ensure that it hybridizes to the target sequence comprising the adenosine to be deaminated.

In some embodiments, the guide sequence is about 10 nt to about 100 nt long and hybridizes to the target DNA strand to form an almost perfectly matched duplex, except for having a dA-C mismatch at the target adenosine site. Particularly, in some embodiments, the dA-C mismatch is located close to the center of the target sequence (and thus the center of the duplex upon hybridization of the guide sequence to the target sequence), thereby restricting the nucleotide deaminase to a narrow editing window (e.g., about 4 bp wide). In some embodiments, the target sequence may comprise more than one target adenosine to be deaminated. In further embodiments, the target sequence may further comprise one or more dA-C mismatch 3′ to the target adenosine site. In some embodiments, to avoid off-target editing at an unintended Adenine site in the target sequence, the guide sequence can be designed to comprise a non-pairing Guanine at a position corresponding to said unintended Adenine to introduce a dA-G mismatch, which is catalytically unfavorable for certain nucleotide deaminases such as ADAR1 and ADAR2. Sec Wong et al., RNA 7:846-858 (2001), which is incorporated herein by reference in its entirety.

In some embodiments, the sequence of the guide molecule (direct repeat and/or spacer) is selected to reduce the degree secondary structure within the guide molecule. In some embodiments, about or less than about 75%, 50%, 40%, 30%, 25%, 20%, 15%, 10%, 5%), 1%), or fewer of the nucleotides of the nucleic acid-targeting guide RNA participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and P A Carr and G M Church, 2009, Nature Biotechnology 27(12): 1151-62).

In some embodiments, it is of interest to reduce the susceptibility of the guide molecule to RNA cleavage, such as to cleavage by Cas7-11. Accordingly, in particular embodiments, the guide molecule is adjusted to avoid cleavage by Cas7-11 or other RNA-cleaving enzymes.

In some embodiments, the guide molecule is modified, e.g., by one or more aptamer(s) designed to improve guide molecule delivery, including delivery across the cellular membrane, to intracellular compartments, or into the nucleus. Such a structure can include, either in addition to the one or more aptamer(s) or without such one or more aptamer(s), moiety(ies) so as to render the guide molecule deliverable, inducible or responsive to a selected effector. The invention accordingly comprehends a guide molecule that responds to normal or pathological physiological conditions, including without limitation pH, hypoxia, O₂ concentration, temperature, protein concentration, enzymatic concentration, lipid structure, light exposure, mechanical disruption (e.g. ultrasound waves), magnetic fields, electric fields, or electromagnetic radiation.

### Adenosine Deaminase

The system may further comprise an adenosine deaminase or catalytic domain thereof. The adenosine deaminase protein or catalytic domain thereof deaminates an Adenine or Cytidine at the one or more codons thereby changing the codon to encode for an amino acid that is not post-translationally modified. The term “adenosine deaminase” or “adenosine deaminase protein” as used herein refers to a protein, a polypeptide, or one or more functional domain(s) of a protein or a polypeptide that is capable of catalyzing a hydrolytic deamination reaction that converts an adenine (or an adenine moiety of a molecule) to a hypoxanthine (or a hypoxanthine moiety of a molecule), as shown below. In some embodiments, the adenine-containing molecule is an adenosine (A), and the hypoxanthine-containing molecule is an inosine (I). The adenine-containing molecule can be deoxyribonucleic acid (DNA) or ribonucleic acid (RNA).

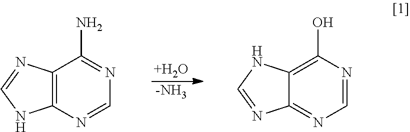

According to the present disclosure, adenosine deaminases that can be used in connection with the present disclosure include, but are not limited to, members of the enzyme family known as adenosine deaminases that act on RNA (ADARs), members of the enzyme family known as adenosine deaminases that act on tRNA (ADATs), and other adenosine deaminase domain-containing (AD AD) family members. According to the present disclosure, the adenosine deaminase is capable of targeting adenine in a RNA/DNA and RNA duplexes. Indeed, Zheng et al. (Nucleic Acids Res. 2017, 45(6): 3369-3377) demonstrate that ADARs can carry out adenosine to inosine editing reactions on RNA/DNA and RNA/RNA duplexes. The adenosine deaminase can be modified to increase its ability to edit DNA in a RNA/DNAn RNA duplex.

In some embodiments, the adenosine deaminase is derived from one or more metazoa species, including but not limited to, mammals, birds, frogs, squids, fish, flies and worms. In some embodiments, the adenosine deaminase is a human, cephalopod (e.g., squid) or Drosophila adenosine deaminase. In certain examples, the adenosine deaminase is a human adenosine deaminase. In certain examples, the adenosine deaminase is a cephalopod adenosine deaminase. In certain examples, the adenosine deaminase is a Drosophila adenosine deaminase.

### Cytidine Deaminase

The term “cytidine deaminase” or “cytidine deaminase protein” as used herein refers to a protein, a polypeptide, or one or more functional domain(s) of a protein or a polypeptide that is capable of catalyzing a hydrolytic deamination reaction that converts a cytosine (or a cytosine moiety of a molecule) to an uracil (or an uracil moiety of a molecule), as shown below. In some embodiments, the cytosine-containing molecule is a cytidine (C), and the uracil-containing molecule is an uridine (U). The cytosine-containing molecule can be deoxyribonucleic acid (DNA) or ribonucleic acid (RNA).

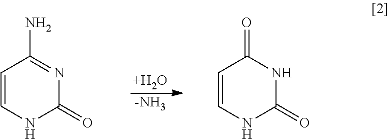

According to the present disclosure, cytidine deaminases that can be used in connection with the present disclosure include, but are not limited to, members of the enzyme family known as apolipoprotein B mRNA-editing complex (APOBEC) family deaminase, an activation-induced deaminase (AID), or a cytidine deaminase 1 (CDA1). In particular embodiments, the deaminase in an APOBEC 1 deaminase, an APOBEC2 deaminase, an APOBEC3A deaminase, an APOBEC3B deaminase, an APOBEC3C deaminase, and APOBEC3D deaminase, an APOBEC3E deaminase, an APOBEC3F deaminase an APOBEC3G deaminase, an APOBEC3H deaminase, or an APOBEC4 deaminase. The cytidine deaminase can be modified to increase its ability to edit DNA in a RNA/DNAn RNA duplex.

In some embodiments, the cytidine deaminase is derived from one or more metazoa species, including but not limited to, mammals, birds, frogs, squids, fish, flies and worms. In some embodiments, the cytidine deaminase is a human, primate, cow, dog, rat or mouse cytidine deaminase.

CD (cytidine deaminase)-functionalized CRISPR system for RNA editing can be used for C to U conversions. In some embodiments, the cytidine deaminase protein or catalytic domain thereof is a human, rat or lamprey cytidine deaminase protein or catalytic domain thereof. In some embodiments, the cytidine deaminase protein or catalytic domain thereof is an apolipoprotein B mRNA-editing complex (APOBEC) family deaminase, an activation-induced deaminase (AID), or a cytidine deaminase 1 (CDA1). In some embodiments, the cytidine deaminase protein or catalytic domain thereof is an APOBEC1 deaminase comprising one or more mutations corresponding to W90A, W90Y, R118A, H121R, H122R, R126A, R126E, or R132E in rat APOBEC1, or an APOBEC3G deaminase comprising one or more mutations corresponding to W285A, W285Y, R313A, D316R, D317R, R320A, R320E, or R326E in human APOBEC3G. In some embodiments, the cytidine deaminase protein or catalytic domain thereof is delivered together with an uracil glycosylase inhibitor (UGI), where said UGI is covalently linked to said cytidine deaminase protein or catalytic domain thereof and/or said catalytically inactive Cas7-11 protein.

Cas7-11-APOBEC fusions can perform C-to-U editing of RNA. APOBEC substrates are ssRNA and the Cas7-11-APOBEC can therefore target regions of the RNA around the guide/target duplex. Cas7-11-APOBEC fusions can perform C to U knockdown via stop codon introduction. In addition to correcting pathogenic U to C mutations that arise during the cellular life cycle, Cas7-11-APOBEC fusions can lead to the introduction of stop codons by converting a CAA, CGA, or CAG to TAA, TGA, or TAG, respectively. APOBEC orthologs in fusion with Cas7-11 can increase the efficiency of C-to-U editing, or can allow for additional types of base conversions. Mutating the APOBEC from the Cas7-11-APOBEC can lead to fusions with specific dsRNA activity, base flip activity and increased activity.

In certain example embodiments, the deaminase is selected from Table 2.

TABLE 2

| SEQ ID NO & | |
|---|---|
| Deaminase Name | Protein Sequence |
| SEQ ID NO: 33 | QLHLPQVLADAVSRLVLGKFGDLTDNFSSPHARRKVLAGVVMTTGTDVKDAKV |
| Homo | ISVSTGTKCINGEYMSDRGLALNDCHAEIISRRSLLRFLYTQLELYLNNKDDQKRS |
| sapiens_ADAR2_E_Q_ | IFQKSERGGFRLKENVQFHLYISTSPCGDARIFSPHEPILEEPADRHPNRKARGQLR |
| Mutant | TKIESGQGTIPVRSNASIQTWDGVLQGERLLTMSCSDKIARWNVVGIQGSLLSIFV |
| | EPIYFSSIILGSLYHGDHLSRAMYQRISNIEDLPPLYTLNKPLLSGISNAEARQPGKA |
| | PNFSVNWTVGDSAIEVINATTGKDELGRASRLCKHALYCRWMRVHGKVPSHLL |
| | RSKITKPNVYHESKLAAKEYQAAKARLFTAFIKAGLGAWVEKPTEQDQFSLT* |
| SEQ ID NO: 34 | SLGTGNRCVKGDSLSLKGETVNDCHAEIISRRGFIRFLYSELMKYNSQTAKDSIFE |
| Homo | PAKGGEKLQIKKTVSFHLYISTAPCGDGALFDKSCSDRAMESTESRHYPVFENPK |
| sapiens_ADARI_E_Q_ | QGKLRTKVENGQGTIPVESSDIVPTWDGIRLGERLRTMSCSDKILRWNVLGLQGA |
| Mutant | LLTHFLQPIYLKSVTLGYLFSQGHLTRAICCRVTRDGSAFEDGLRHPFIVNHPKVG |
| | RVSIYDSKRQSGKTKETSVNWCLADGYDLEILDGTRGTVDGPRNELSRVSKKNIF |
| | LLFKKLCSFRYRRDLLRLSYGEAKKAARDYETAKNYFKKGLKDMGYGNWISKP |
| | QEEKNF |
| SEQ ID NO: 35 | SVGTGNRCLTGDHLSLEGNSVNDSHAEMITRRGFLRYLYRHLLEYDAEVPNDLF |
| Octopus | EKGERSICRIKTNITFHLYISTAPCGDGALFSPRDTDSSNAKMEEENKHIHNPTFSS |
| vulgaris_ADARI_E_Q_ | SVQGLLRTKVEGGQGTIPIDADFTEQTWDGIQRGERLRTMSCSDKICRWNVVGL |
| Mutant | QGALLSHFIEPIYLDSLTLGYLYDHGHLARAVCCRIERGEASVNQLLPEGYRLNH |
| | PWLGRVTACDPPRETQKTKSLSINWCYDDEKSEVLDGTAGICYTAIEKNLFSRLT |
| | KHNLYEEFKRVCRKFDRNDLLTAPSYNKAKMMATPFQTAKNVMLKKLKENNC |
| | GTWVSKPIEEEMF |
| SEQ ID NO: 36 | SVGTGNRCLTGDRLSLEGNSVNDSHAEMVTRRGFLRYLYKHLLEYDPEKPHDLF |
| Sepia_ADARI_E_Q_ | EKGERSLCRIKTNITFHLYISTAPCGDGALFSPRDTDSSNVKVDEENKHVHNPTFS |
| Mutant | SSVQGLLRTKVEGGQGTIPIDADFTEQTWDGIQRGERLRTMSCSDKICRWNVVGL |
| | QGALLSHFVEPIYLESLTLGYLYDHGHLARAVCCRIERGEASVNQLLPEGYRLNH |
| | PWLGRVTACDPPRETQKTKSLSINWCYDDEKSEVLDGTAGICYTAIEKNLFSRLT |
| | KHSLYEEFKKVCQKFEREDLLNVTSYNKAKMMAIPFQTAKNVMLKKLKENNCG |
| | TWVSKPIEEEMF |
| SEQ ID NO: 37 | GIGTGTKCINGEHMSDRGFGVNDCHAEIIARRCFLRYIYDQLELHLSDNSDVRNSS |
| Octopus | IFELRDKGGYQLKENIQFHLYISTAPCGDARIFSPHGQDVETGDRHPNRKARGQL |
| vulgaris_ADAR2_E_Q_ | RTKIESGQGTIPVRTSGVIQTWDGVLEGERLLTMSCSDKIARWNVLGIQGSLLSHF |
| Mutant | MNPIYLESIILGSLYHSDHLSRAMYSRISIIENLPEPFHLNRPFLSGISSPESRQPGKA |
| | PNFGINWRKEDETFEVINAMTGRVEGGSVSRICKQALFGRFMSLYGKLSSLTGQS |
| | VTTRPTHYSDAKAAVMEYQLAKQCVFQAFQKAGLGNWVQKPIEQDQF |
| SEQ ID NO: 38 | GIGTGTKCINGEYMNDRGFAVNDCHAEIIARRCFLRFIYDQLEMHLSEDPEVRGQ |
| Sepia_ADAR2_E_Q_ | SVFELRDGGGYKLKPNIHFHLYISTAPCGDARIFSPHGQDVETGDRHPNRKARGQ |
| Mutant | LRTKIESGQGTIPVRSSGFIQTWDGVLEGERLLTMSCSDKIARWNVLGIQGALLCH |
| | FMHPIYLESIILGSLYHSDHLSRAVYCRIASIENLPDLFQLNRPFLSGISSPESRQPG |
| | KAPNFGINWRRNDDTFEVINAMTGRVEGGNMSRICKQALFDRFMNLYGRLSSLT |
| | GQSVTTRPTLYSEAKAAVMEYQLAKQCVFQAFQKAGLGNWVQKPIEQDQF |
| SEQ ID NO: 39 | GIGTGTKCINGEYMNDRGFAVNDCHAEIIARRCFLRFIYDQLELHLSDNAEVRGQ |
| Doryteusthis | SIFELRDAGGYKLKPNIQFHLYISTAPCGDARIFSPHGQDVETGDRHPNRKARGQL |
| opalescens_ADAR2_E_ | RTKIESGQGTIPVRSSGFIQTWDGVLEGERLLTMSCSDKIARWNVLGVQGALLCH |
| Q_Mutant | FMHPIYLESIILGSLYHSDHLSRAVYCRIAAIENLPDLFRLNRPFLSGISSPESRQPG |
| | KAPNFGINWRRNDDSFEVINAMTGRVEGGSMSRICKQALFDRFMNLYGKLSSLT |
| | GQSVTTRPALYSEAKATVMEYQLAKQCVFQAFQKAGLGNWVQKPIEQDQF |
| SEQ ID NO: 40 | MTSEKGPSTGDPTLRRRIEPWEFDVFYDPRELRKEACLLYEIKWGMSRKIWRSSG |
| APOBECI | KNTTNHVEVNFIKKFTSERDFHPSMSCSITWFLSWSPCWECSQAIREFLSRHPGVT |
| | LVIYVARLFWHMDQQNRQGLRDLVNSGVTIQIMRASEYYHCWRNFVNYPPGDE |
| | AHWPQYPPLWMMLYALELHCIILSLPPCLKISRRWQNHLTFFRLHLQNCHYQTIP |
| | PHILLATGLIHPSVAWR |
| SEQ ID NO: 41 | MAQKEEAAVATEAASQNGEDLENLDDPEKLKELIELPPFEIVTGERLPANFFKFQ |
| APOBEC2 | FRNVEYSSGRNKTFLCYVVEAQGKGGQVQASRGYLEDEHAAAHAEEAFFNTILP |
| | AFDPALRYNVTWYVSSSPCAACADRIIKTLSKTKNLRLLILVGRLFMWEEPEIQA |
| | ALKKLKEAGCKLRIMKPQDFEYVWQNFVEQEEGESKAFQPWEDIQENFLYYEEK |
| | LADILK |
| SEQ ID NO: 42 | MEASPASGPRHLMDPHIFTSNFNNGIGRHKTYLCYEVERLDNGTSVKMDQHRGF |
| APOBEC3A | LHNQAKNLLCGFYGRHAELRFLDLVPSLQLDPAQIYRVTWFISWSPCFSWGCAG |
| | EVRAFLQENTHVRLRIFAARIYDYDPLYKEALQMLRDAGAQVSIMTYDEFKHCW |
| | DTFVDHQGCPFQPWDGLDEHSQALSGRLRAILQNQGN |
| SEQ ID NO: 43 | MNPQIRNPMERMYRDTFYDNFENEPILYGRSYTWLCYEVKIKRGRSNLLWDTGV |
| APOBEC3B | FRGQVYFEPQYHAEMCFLSWFCGNQLPAYKCFQITWFVSWTPCPDCVAKLAEFL |
| | SEHPNVTLTISAARLYYYWERDYRRALCRLSQAGARVKIMDYEEFAYCWENFV |
| | YNEGQQFMPWYKFDENYAFLHRTLKEILRLRIFSVAFTAAMRSCASWTWFLLCS |
| | WTRPRSTGSLGSSPGAPASPGAVPGKCVRSFRRTHT |
| SEQ ID NO: 44 | MNPQIRNPMKAMYPGTFYFQFKNLWEANDRNETWLCFTVEGIKRRSVVSWKTG |
| APOBEC3C | VFRNQVDSETHCHAERCFLSWFCDDILSPNTKYQVTWYTSWSPCPDCAGEVAEF |
| | LARHSNVNLTIFTARLYYFQYPCYQEGLRSLSQEGVAVEIMDYEDFKYCWENFV |
| | YNDNEPFKPWKGLKTNFRLLKRRLRESLQ |
| SEQ ID NO: 45 | MNPQIRNPMERMYRDTFYDNFENEPILYGRSYTWLCYEVKIKRGRSNLLWDTGV |
| APOBEC3D | FRGPVLPKRQSNHRQEVYFRFENHAEMCFLSWFCGNRLPANRRFQITWFVSWNP |
| | CLPCVVKVTKFLAEHPNVTLTISAARLYYYRDRDWRWVLLRLHKAGARVKIMD |
| | YEGERCRGQGSMTGRNSLRDGWICNAMAGGVPGQPAGVGLALIATDSQETRPG |
| | RAGPGSGESLSASHLFISDFAYCWENFVCNEGQPFMPWYKFDDNYASLHRTLKEI |
| | LRNPMEAMYPHIFYFHFKNLLKACGRNESWLCFTMEVTKHHSAVFRKRGVFRN |
| | QVDPETHCHAERCFLSWFCDDILSPNTNYEVTWYTSWSPCPECAGEVAEFLARH |
| | SNVNLTIFTARLCYFWDTDYQEGLCSLSQEGASVKIMGYKDFVSCWKNFVYSDD |
| | EPFKPWKGLQTNFRLLKRRLREILQ |
| SEQ ID NO: 46 | MKPHFRNTVERMYRDTFSYNFYNRPILSRRNTVWLCYEVKTKGPSRPRLDAKIF |
| APOBEC3F | RGQVYSQPEHHAEMCFLSWFCGNQLPAYKCFQITWFVSWTPCPDCVAKLAEFL |
| | AEHPNVTLTISAARLYYYWERDYRRALCRLSQAGARVKIMDDEEFAYCWENFV |
| | YSEGQPFMPWYKFDDNYAFLHRTLKEILRNPMEAMYPHIFYFHFKNLRKAYGRN |
| | ESWLCFTMEVVKHHSPVSWKRGVFRNQVDPETHCHAERCFLSWFCDDILSPNTN |
| | YEVTWYTSWSPCPECAGEVAEFLARHSNVNLTIFTARLYYFWDTDYQEGLRSLS |
| | QEGASVEIMGYKDFKYCWENFVYNDDEPFKPWKGLKYNFLFLDSKLQEILE |
| SEQ ID NO: 47 | MELKYHPEMRFFHWFSKWRKLHRDQEYEVTWYISWSPCTKCTRDMATFLAEDP |
| APOBEC3G | KVTLTIFVARLYYFWDPDYQEALRSLCQKRDGPRATMKIMNYDEFQHCWSKFV |
| | YSQRELFEPWNNLPKYYILLHIMLGEILRHSMDPPTFTFNFNNEPWVRGRHETYL |
| | CYEVERMHNDTWVLLNQRRGFLCNQAPHKHGFLEGRHAELCFLDVIPFWKLDL |
| | DQDYRVTCFTSWSPCFSCAQEMAKFISKNKHVSLCIFTARIYDDQGRCQEGLRTL |
| | AEAGAKISIMTYSEFKHCWDTFVDHQGCPFQPWDGLDEHSQDLSGRLRAILQNQ |
| | EN |
| SEQ ID NO: 48 | MALLTAETFRLQFNNKRRLRRPYYPRKALLCYQLTPQNGSTPTRGYFENKKKCH |
| APOBEC3H | AEICFINEIKSMGLDETQCYQVTCYLTWSPCSSCAWELVDFIKAHDHLNLRIFASR |
| | LYYHWCKPQQDGLRLLCGSQVPVEVMGFPDSRGTCAGSLHGYIV |
| SEQ ID NO: 49 | MEPIYEEYLANHGTIVKPYYWLSFSLDCSNCPYHIRTGEEARVSLTEFCQIFGFPY |
| APOBEC4 | GTTFPQTKHLTFYELKTSSGSLVQKGHASSCTGNYIHPESMLFEMNGYLDSAIYN |
| | NDSIRHIILYSNNSPCNEANHCCISKMYNFLITYPGITLSIYFSQLYHTEMDFPASA |
| | WNREALRSLASLWPRVVLSPISGGIWHSVLHSFISGVSGSHVFQPILTGRALADRH |
| | NAYEINAITGVKPYFTDVLLQTKRNPNTKAQEALESYPLNNAFPGQFFQMPSGQL |
| | QPNLPPDLRAPVVFVLVPLRDLPPMHMGQNPNKPRNIVRHLNMPQMSFQETKDL |
| | GRLPTGRSVEIVEITEQFASSKEADEKKKKKGKK |
| SEQ ID NO: 50 | MDSLLMNRKFLYQFKNVRWAKGRRETYLCYVVERRDSATSFSLDFGYLRNKNG |
| Activation-induced | CHVELLFLRYISDWDLDPGRCYRVTWFTSWSPCYDCARHVADFLRGNPNLSLRI |
| cytidine deaminase | FTARLYFCEDRKAEPEGLRRLHRAGVQIAIMTFKDYFYCWNTFVENHERTFKAW |
| (AID) | BGLHENSVRLSRQLRRILL |

### EXAMPLES

While several experimental Examples are contemplated, these Examples are intended non-limiting.

### Materials

Table 3 below shows examples of bacterial plasmids.

TABLE 3

| Plasmid | | |
|---|---|---|
| number | Name | Benchling link |
| pDF0038 | DsiCas7-11a full locus with CRISPR array | https://benchling.com/s/seq- |
| | | 72T3bYt8Ff9QNtG7aE2X |
| pDF0039 | DsiCas7-11a only expression with | https://benchling.com/s/seq- |
| | CRISPR array | uiS8cQyZLi2NrqfhxWF2 |
| pDF0044 | CjcCas7-11b full locus with synthetic array | https://benchling.com/s/seq- |
| | | GXIXQfFqwbN2vu7g6NUV |
| pDF0118 | DsiCas7-11a bacterial expression with | https://benchling.com/s/seq- |
| | Twinstrep-SUMO-Codon | PSpbXxlK66yLBw0TbxnY |
| pDF0163 | GwCas7-11c bacterial expression with | https://benchling.com/s/seq- |
| | Twinstrep-SUMO-Codon | CsMaJRrLeJ15XtHvMFd1 |
| pDF0191 | CjcCas7-11b bacterial expression with | https://benchling.com/s/seq- |
| | Twinstrep-SUMO-Codon | lLOOF2Yl8T6v2jUuQ9Q3 |
| pDF0229 | DsiCas7-11a expression vcetor with | https://benchling.com/s/seq- |
| | golden gate site for guides | mPWeEpoNVZVKxJwlIhVj |

Table 4 below shows examples of bacterial spacers.

TABLE 4

| SEQ ID NO | Spacer sequence | Target |
|---|---|---|
| SEQ ID NO: 51 | CTTTGTTGTCTTCGACATGGGTAATCCTCAT | MS2 targeting 1 |
| SEQ ID NO: 52 | TAAAGAGTTGAACTTCTTTGTTGTCTTCGAC | MS2 targeting 3 |
| SEQ ID NO: 53 | GTACCTTCGTACGGACGACCTTCACCTTCAC | RFP targeting 1 |
| SEQ ID NO: 54 | CAGGATGTCCCAAGCGAACGGCAGCGGACC | RFP targeting 2 |
| | A | |
| SEQ ID NO: 55 | CAGGGAGGAGTCCTGGGTAACGGTAACAAC | RFP targeting 3 |
| | A | |
| SEQ ID NO: 56 | CATGAACTCTTTGATAACGTCTTCGCTACTC | RFP targeting 4 |
| SEQ ID NO: 57 | TCGGAGGTAGCCCGACTCGCTCCGGTTCGTA | non-targeting 1 |
| SEQ ID NO: 58 | TGAAAGTTGGCGCCTTTTTTCATACGTCGAG | non-targeting 2 |

Table 5 below shows examples of targets and pre-crRNA.

TABLE 5

| Target | Sequence |
|---|---|
| SEQ ID NO: 59 | GGGCGAAAUCGGUAUUUCCAAUGAAAUCACUCAGGUUGGAAAGCCGGUUUUC |
| DsiCas7-11 array | UUUGAUGUCACGGAACCGCCAAUAUAACAAUACAGGUAUUAUAGUUGGGUUG |
| (4DR-3S) | GAAAGCCGGUUUUCUUUGAUGUCACGGAACAUUGUAAACUGCAUAUCUGUGU |
| | UUGCCGUCUGGUUGGAAAGCCGGUUUUCUUUGAUGUCACGGAACUCACGUGA |
| | GUCAGUCUUAGUUCUAUACUUCUUUAGGGUUGGAAAGCCGGUUUUCUUUGAU |
| | GUCACGGAACAGGAACUUGAACAACAUCGUUACUAACGAG |
| SEQ ID NO: 60 | GGGGGCCAGUGAAUUCGAGCUCGGUACCCGGGGAUCCUCUAGAAAUAUGGAU |
| ssRNA 1 target | UACUUGGUAGAACAGCAAUCUACUCGACCUGCAGGCAUGCAAGCUUGGCGUA |
| | AUCAUGGUCAUAGCUGUUUCCUGUGUUUAUCCGCUCACAAUUCCACACAACA |
| | UACGAGCCGGAAGCAUAAAG |
| SEQ ID NO: 61 | GGGGCAAACUCCGGCAUCUACUAAUAGACGCCGGCCAUUCAAACAUGAGGAU |
| MS2 target for | UACCCAUGUCGAAGACAACAAAGAAGUUCAACUCUUUAUGUAUUGAUCUUCC |
| body labeling | UCGCGAUCUUUCUCUCGAAAUUUACCAAUCAAUUGCUUCUGUCGCUACU |
| SEQ ID NO: 62 | GGGGGCCAGUGAAUUCGAGCUCGGUACCCGGGGAUCCUCUAGAAAUAUGGAU |
| SSRNA 2 target | UACUUGGUAGAACAGCAAUCUACUCGACCUGCAGGCAUGCAAGCUUGGCGUA |
| | AUCAUGGUCAUAGCUGUUUCCUGUGUUUAUCCGCUCACAAUUCCACACA |
| SEQ ID NO: 63 | GGCCAGUGAAUUCGAGCUCGGUACCCGGGGAUCCUCUAGAAAUAUGGAUUAC |
| fixed crRNA | UUGGGUCGAAGACAACAAAGAAGUUCAACUCUUUAUAGAACAGCAAUCUACU |
| target 1 | CGACCUGCAGGCAUGCAAGCUUGGCGUAAUCAUGGUCAUAGCUGUU |
| SEQ ID NO: 64 | AUGCCCGCCAUGAAGAUCGAGUGCCGCAUCACCGGCACCCUGAACGGCGUGGA |
| fixed crRNA | GUUGUCGAAGACAACAAAGAAGUUCAACUCUUUACGAGCUGGUGGGCGGCGG |
| target 2 | AGAGGGCACCCCCGAGCAGGGCCGCAUGACCAACAAGAUGAAGAG |
| SEQ ID NO: 65 | AUUCUAUGUCUGGUUUUCGCUCAAAAACUUCCCGGAAAUGACAACAGCACGG |
| fixed crRNA | CAACGUCGAAGACAACAAAGAAGUUCAACUCUUUAGCUGUGCCUUGGGCACC |
| target 3 | AUGCAGUACCAAACGGAACGAUAGUGAAAACAAUCACGAAUGACCA |
| SEQ ID NO: 66 | AUUCUAUGUCUGGUUUUCGCUCAAAAACUUCCCGGAAAUGACAACAGCACGG |
| fixed crRNA | CAACGUCGAAGACAACAAAGAAGUUCAACUCUUUAGCUGUGCCUUGGGCACC |
| target 4 | AUGCAGUACCAAACGGAACGAUAGUGAAAACAAUCACGAAUGACCA |
| SEQ ID NO: 67 | GCUGUGCCUUGGGCACCAUGCAGUACCAAACGGAACGAUAGUGAAAACAAUC |
| fixed crRNA | ACGAAUGACCAGUCGAAGACAACAAAGAAGUUCAACUCUUUAAUUCUAUGUC |
| target 5 | UGGUUUUCGCUCAAAAACUUCCCGGAAAUGACAACAGCACGGCAAC |
| SEQ ID NO: 68 | GGGGUUUGGAAGCCGGGUCGAAGUCAGGCCCGUUAAGACCGAGUAGAUUGCU |
| GwCas7-11 | GUUCUACCAAGUAAUCCAGUUUGGAAGCCGGGUCGAAGUCAGGCCCGUUAAG |
| array (3DR-2S) | ACUAUGCUUCCGGCUCGUAUGUUGUGUGGAAUUGUUUGGAAGCCGGGUCGAA |
| | GUCAGGCCCGUUAAGAC |
| SEQ ID NO: 69 | GGGGCUAGCUCAGUCCUAGGUAUAAUGCUAGCGCUGAUUUAGGCAAAAACGG |
| CjcCas7-11 array | CUUGAAGACUAAAGGAAGGAAUUAAUGUCACGGUACGACUAAAUGGCACCAC |
| (3DR-2S) | UAAUUGUUGCCUUCUAAGUUCCUUCUUGAAGACUAAAGGAAGGAAUUAAUGU |
| | CACGGUACGUUUGUUUAACCACAUGAGGCACACCGCCCUCGUGAGCUUGAAG |
| | ACUAAAGGAAGGAAUUAAUGUCACGGUACAAUCUCCGCCCCGUUCGUAAGC |
| SEQ ID NO: 70 | AUGCCCGCCAUGAAGAUCGAGUGCCGCAUCACCGGCACCCUGAACGGCGUGGA |
| EGFP ssRNA | GUUCGAGCUGGUGGGCGGCGGAGAGGGCACCCCCGAGCAGGGCCGCAUGACC |
| 400 nt | AACAAGAUGAAGAGCACCAAAGGCGCCCUGACCUUCAGCCCCUACCUGCUGAG |
| | CCACGUGAUGGGCUACGGCUUCUACCACUUCGGCACCUACCCCAGCGGCUACG |
| | AGAACCCCUUCCUGCACGCCAUCAACAACGGCGGCUACACCAACACCCGCAUC |
| | GAGAAGUACGAGGACGGCGGCGUGCUGCACGUGAGCUUCAGCUACCGCUACG |
| | AGGCCGGCCGCGUGAUCGGCGACUUCAAGGUGGUGGGCACCGGCUUCCCCGAG |
| | GACAGCGUGAUCUUCACCGACAAGAUCAUCC |
| SEQ ID NO: 71 | GGGGUUGGAAAGCCGGUUUUCUUUGAUGUCACGGAACCUUUGUUGUCUUCGA |
| MS2 crRNA 1 | CAUGGGUAAUCCUCAUGGUUGGAAAGCCGGUUUUCUUUGAUGUCACGGAAC |
| (mini array) WT | |
| SEQ ID NO: 72 | GGCGUUGGAAAGCCGGUUUUCUUUGAUGUCACGGAACCUUUGUUGUCUUCGA |
| MS2 crRNA 1 | CAUGGGUAAUCCUCAUCGUUGGAAAGCCGGUUUUCUUUGAUGUCACGGAAC |
| (mini array) | |
| mutant pos 1 | |
| SEQ ID NO: 73 | GGGGAUGGAAAGCCGGUUUUCUUUGAUGUCACGGAACCUUUGUUGUCUUCGA |
| MS2 crRNA 1 | CAUGGGUAAUCCUCAUGGAUGGAAAGCCGGUUUUCUUUGAUGUCACGGAAC |
| (mini array) | |
| mutant pos 3 | |
| SEQ ID NO: 74 | GGGGUUCGAAAGCCGGUUUUCUUUGAUGUCACGGAACCUUUGUUGUCUUCGA |
| MS2 crRNA 1 | CAUGGGUAAUCCUCAUGGUUCGAAAGCCGGUUUUCUUUGAUGUCACGGAAC |
| (mini array) | |
| mutant pos 5 | |
| SEQ ID NO: 75 | GGGGUUGGUAAGCCGGUUUUCUUUGAUGUCACGGAACCUUUGUUGUCUUCGA |
| MS2 crRNA 1 | CAUGGGUAAUCCUCAUGGUUGGUAAGCCGGUUUUCUUUGAUGUCACGGAAC |
| (mini array) | |
| mutant pos 7 | |
| SEQ ID NO: 76 | GGGGUUGGAAUGCCGGUUUUCUUUGAUGUCACGGAACCUUUGUUGUCUUCGA |
| MS2 crRNA 1 | CAUGGGUAAUCCUCAUGGUUGGAAUGCCGGUUUUCUUUGAUGUCACGGAAC |
| (mini array) | |
| mutant pos 9 | |
| SEQ ID NO: 77 | GGGGUUGGAAAGGCGGUUUUCUUUGAUGUCACGGAACCUUUGUUGUCUUCGA |
| MS2 crRNA 1 | CAUGGGUAAUCCUCAUGGUUGGAAAGGCGGUUUUCUUUGAUGUCACGGAAC |
| (mini array) | |
| mutant pos 11 | |
| SEQ ID NO: 78 | GGGGUUGGAAAGCCCGUUUUCUUUGAUGUCACGGAACCUUUGUUGUCUUCGA |
| MS2 crRNA 1 | CAUGGGUAAUCCUCAUGGUUGGAAAGCCCGUUUUCUUUGAUGUCACGGAAC |
| (mini array) | |
| mutant pos 13 | |
| SEQ ID NO: 79 | GGGGUUGGAAAGCCGGAUUUCUUUGAUGUCACGGAACCUUUGUUGUCUUCGA |
| MS2 crRNA 1 | CAUGGGUAAUCCUCAUGGUUGGAAAGCCGGAUUUCUUUGAUGUCACGGAAC |
| (mini array) | |
| mutant pos 15 | |
| SEQ ID NO: 80 | GGGGUUGGAAAGCCGGUUAUCUUUGAUGUCACGGAACCUUUGUUGUCUUCGA |
| MS2 crRNA 1 | CAUGGGUAAUCCUCAUGGUUGGAAAGCCGGUUAUCUUUGAUGUCACGGAAC |
| (mini array) | |
| mutant pos 17 | |
| SEQ ID NO: 81 | GGGGUUGGAAAGCCGGUUUUGUUUGAUGUCACGGAACCUUUGUUGUCUUCGA |
| MS2 crRNA 1 | CAUGGGUAAUCCUCAUGGUUGGAAAGCCGGUUUUGUUUGAUGUCACGGAAC |
| (mini array) | |
| mutant pos 19 | |
| SEQ ID NO: 82 | GGGGUUGGAAAGCCGGUUUUCUAUGAUGUCACGGAACCUUUGUUGUCUUCGA |
| MS2 crRNA 1 | CAUGGGUAAUCCUCAUGGUUGGAAAGCCGGUUUUCUAUGAUGUCACGGAAC |
| (mini array) | |
| mutant pos 21 | |
| SEQ ID NO: 83 | GGGGUUGGAAAGCCGGUUUUCUUUCAUGUCACGGAACCUUUGUUGUCUUCGA |
| MS2 crRNA 1 | CAUGGGUAAUCCUCAUGGUUGGAAAGCCGGUUUUCUUUCAUGUCACGGAAC |
| (mini array) | |
| mutant pos 23 | |
| SEQ ID NO: 84 | GGGGUUGGAAAGCCGGUUUUCUUUGAAGUCACGGAACCUUUGUUGUCUUCGA |
| MS2 crRNA 1 | CAUGGGUAAUCCUCAUGGUUGGAAAGCCGGUUUUCUUUGAAGUCACGGAAC |
| (mini array) | |
| mutant pos 25 | |
| SEQ ID NO: 85 | GGGGUUGGAAAGCCGGUUUUCUUUGAUGACACGGAACCUUUGUUGUCUUCGA |
| MS2 crRNA 1 | CAUGGGUAAUCCUCAUGGUUGGAAAGCCGGUUUUCUUUGAUGACACGGAAC |
| (mini array) | |
| mutant pos 27 | |
| SEQ ID NO: 86 | GGGGUUGGAAAGCCGGUUUUCUUUGAUGUCUCGGAACCUUUGUUGUCUUCGA |
| MS2 crRNA 1 | CAUGGGUAAUCCUCAUGGUUGGAAAGCCGGUUUUCUUUGAUGUCUCGGAAC |
| (mini array) | |
| mutant pos 29 | |
| SEQ ID NO: 87 | GGGGUUGGAAAGCCGGUUUUCUUUGAUGUCACCGAACCUUUGUUGUCUUCGA |
| MS2 crRNA 1 | CAUGGGUAAUCCUCAUGGUUGGAAAGCCGGUUUUCUUUGAUGUCACCGAAC |
| (mini array) | |
| mutant pos 31 | |
| SEQ ID NO: 88 | GGGGUUGGAAAGCCGGUUUUCUUUGAUGUCACGGUACCUUUGUUGUCUUCGA |
| MS2 crRNA 1 | CAUGGGUAAUCCUCAUGGUUGGAAAGCCGGUUUUCUUUGAUGUCACGGUAC |
| (mini array) | |
| mutant pos 33 | |
| SEQ ID NO: 89 | GGGGUUGGAAAGCCGGUUUUCUUUGAUGUCACGGUAGCUUUGUUGUCUUCGA |
| MS2 crRNA 1 | CAUGGGUAAUCCUCAUGGUUGGAAAGCCGGUUUUCUUUGAUGUCACGGUAG |
| (mini array) | |
| mutant pos 35 | |
| SEQ ID NO: 90 | GGGGUUGGAAAGCCGGUUUUCUUUGAUGUCACGGAACCUUUGUUGUCUUCGA |
| MS2 crRNA 1 | CAUGGGUAAUCCUCAUGGUUGGAAAGCCGGUUUUCUUUGAUGUCACGGAAC |
| (mini array) WT | |
| SEQ ID NO: 91 | GGGGUUGGAAAGGGGGUUUUCUUUGAUGUCACGGAACCUUUGUUGUCUUCGA |
| MS2 crRNA 1 | CAUGGGUAAUCCUCAUGGUUGGAAAGGGGGUUUUCUUUGAUGUCACGGAAC |
| (mini array) | |
| double mutant | |
| pos 11 | |
| SEQ ID NO: 92 | GGGGUUGGAAAGCCGCAUUUCUUUGAUGUCACGGAACCUUUGUUGUCUUCGA |
| MS2 crRNA 1 | CAUGGGUAAUCCUCAUGGUUGGAAAGCCGCAUUUCUUUGAUGUCACGGAAC |
| (mini array) | |
| double mutant | |
| pos 14 | |
| SEQ ID NO: 93 | GGGGUUGGAAAGCCGGUUAACUUUGAUGUCACGGAACCUUUGUUGUCUUCGA |
| MS2 crRNA 1 | CAUGGGUAAUCCUCAUGGUUGGAAAGCCGGUUAACUUUGAUGUCACGGAAC |
| (mini array) | |
| double mutant | |
| pos 17 | |
| SEQ ID NO: 94 | GGGGUUGGAAAGCCGGUUUUGAUUGAUGUCACGGAACCUUUGUUGUCUUCGA |
| MS2 crRNA 1 | CAUGGGUAAUCCUCAUGGUUGGAAAGCCGGUUUUGAUUGAUGUCACGGAAC |
| (mini array) | |
| double mutant | |
| pos 19 | |
| SEQ ID NO: 95 | GGGGUUGGAAAGCCGGUUUUCUUACAUGUCACGGAACCUUUGUUGUCUUCGA |
| MS2 crRNA 1 | CAUGGGUAAUCCUCAUGGUUGGAAAGCCGGUUUUCUUACAUGUCACGGAAC |
| (mini array) | |
| double mutant | |
| pos 22 | |
| SEQ ID NO: 96 | GGGGUUGGAAAGCCGGUUUUCUUUGAACUCACGGAACCUUUGUUGUCUUCGA |
| MS2 crRNA 1 | CAUGGGUAAUCCUCAUGGUUGGAAAGCCGGUUUUCUUUGAACUCACGGAAC |
| (mini array) | |
| double mutant | |
| pos 25 | |
| SEQ ID NO: 97 | GGGGUUGGAAAGCCGGUUUUCUUUGAUGUGUCGGAACCUUUGUUGUCUUCGA |
| MS2 crRNA 1 | CAUGGGUAAUCCUCAUGGUUGGAAAGCCGGUUUUCUUUGAUGUGUCGGAAC |
| (mini array) | |
| double mutant | |
| pos 28 | |
| SEQ ID NO: 98 | GGGGUUGGAAAGCCGGUUUUCUUUGAUGUCACCCAACCUUUGUUGUCUUCGA |
| MS2 crRNA 1 | CAUGGGUAAUCCUCAUGGUUGGAAAGCCGGUUUUCUUUGAUGUCACCCAAC |
| (mini array) | |
| double mutant | |
| pos 31 | |
| SEQ ID NO: 99 | GGGGUUGGAAAGCCGGUUUUCUUUGAUGUCACGGAUGCUUUGUUGUCUUCGA |
| MS2 crRNA 1 | CAUGGGUAAUCCUCAUGGUUGGAAAGCCGGUUUUCUUUGAUGUCACGGAUG |
| (mini array) | |
| double mutant | |
| pos 34 | |
| SEQ ID NO: 100 | GGGAAAUCAGCGAAAUGCACCCCGCAUUACGUUUGGUGGACCCUCAGAUUCA |
| SARS-COV2 | ACUGGCAGUAACCAGAAUGGAGAACGCAGUGGGGCGCGAUCAAAACAACGUC |
| SSRNA | GGCCCCAAGGUUUACCCAAUAAUACUGCGUCUUGGUUCACCGCUCUCAC |
| SEQ ID NO: 101 | GGGAUUCUAUGUCUGGUUUUCGCUCAAAAACUUCCCGGAAAUGACAACAGCA |
| Influenza A | CGGCAACGCUGUGCCUUGGGCACCAUGCAGUACCAAACGGAACGAUAGUGAA |
| segment 4 HA | AACAAUCACGAAUGACCAAAUUGAAGUCACUAAUGCUACUGAACUGGUU |
| gene | |
| SEQ ID NO: 102 | GGGATGAGGATTACCCATGTCGAAGACAACAAAG |
| 34-nt MS2 | |
| ssRNA target | |
| SEQ ID NO: 103 | AUGCCCGCCAUGAAGAUCGAGUGCCGCAUCACCGGCACCCUGAACGGCGUGGA |
| EGFP ssRNA | GUUCGAGCUGGUGGGCGGCGGAGAGGGCACCCCCGAGCAGGGCCGCA |
| 100 nt | |
| SEQ ID NO: 104 | AUGCCCGCCAUGAAGAUCGAGUGCCGCAUCACCGGCACCCUGAACGGCGUGGA |
| EGFP ssRNA | GUUCGAGCUGGUGGGCGGCGGAGAGGGCACCCCCGAGCAGGGCCGCAUGACC |
| 200 nt | AACAAGAUGAAGAGCACCAAAGGCGCCCUGACCUUCAGCCCCUACCUGCUGAG |
| | CCACGUGAUGGGCUACGGCUUCUACCACUUCGGCACCUACCC |
| SEQ ID NO: 105 | AUGCCCGCCAUGAAGAUCGAGUGCCGCAUCACCGGCACCCUGAACGGCGUGGA |
| EGFP ssRNA | GUUCGAGCUGGUGGGCGGCGGAGAGGGCACCCCCGAGCAGGGCCGCAUGACC |
| 600 nt | AACAAGAUGAAGAGCACCAAAGGCGCCCUGACCUUCAGCCCCUACCUGCUGAG |
| | CCACGUGAUGGGCUACGGCUUCUACCACUUCGGCACCUACCCCAGCGGCUACG |
| | AGAACCCCUUCCUGCACGCCAUCAACAACGGCGGCUACACCAACACCCGCAUC |
| | GAGAAGUACGAGGACGGCGGCGUGCUGCACGUGAGCUUCAGCUACCGCUACG |
| | AGGCCGGCCGCGUGAUCGGCGACUUCAAGGUGGUGGGCACCGGCUUCCCCGAG |
| | GACAGCGUGAUCUUCACCGACAAGAUCAUCCGCAGCAACGCCACCGUGGAGCA |
| | CCUGCACCCCAUGGGCGAUAACGUGCUGGUGGGCAGCUUCGCCCGCACCUUCA |
| | GCCUGCGCGACGGCGGCUACUACAGCUUCGUGGUGGACAGCCACAUGCACUUC |
| | AAGAGCGCCAUCCACCCCAGCAUCCUGCAGAACGGGGGCCCCAUGUUCGCCUU |
| | CCGCCGCGUGGAGGAGCUG |
| SEQ ID NO: 106 | CCGGCCAUUCAAACAUGAGGAUUACCCAUGUCGAAGACAACAAAGAAGUUCA |
| 60 nt MS2 | ACUCUUUA |
| SSRNA | |
| SEQ ID NO: 107 | GGCCAGUGAAUUCGAGCUCGGUACCCGGGGAUCCUCUAGAAAUAUGGAUUAC |
| ssRNA 1 with | UUGgUAGAACAGCAAUCUACUCGNNNNNNACCUGCAGGCAUGCAAGCUUGGC |
| right PFS | GUAAUCAUGGUCAUAGCUGUUUCCUGUGUUUAUCCGCUCACAAUUCCACACA |
| | ACAUACGAG CCGGAAGCAU |
| SEQ ID NO: 108 | GGCCAGUGAAUUCGAGCUCGGUACCCGGGGAUCCUCUAGAAAUANNNNNNUG |
| ssRNA 1 with | GAUUACUUGgUAGAACAGCAAUCUACUCGACCUGCAGGCAUGCAAGCUUGGCG |
| left PFS | UAAUCAUGGUCAUAGCUGUUUCCUGUGUUUAUCCGCUCACAAUUCCACACAA |
| | CAUACGAGCCGGAAGCAU |
| SEQ ID NO: 109 | GGGAUGAGGAUUACCCAUGUCGAAGACAACAAAG |
| 31-nt MS2 | |
| ssRNA target | |
| SEQ ID NO: 110 | CCGGCATCTACTAATAGACGCCGGCCATTCAAACATGAGGATTACCCATGTCGA |
| MS2 ssDNA and | AGACAACAAAGAAGTTCAACTCTTTATGTATTGATCTTCCTCGCGA |
| dsDNA target | |

Table 6 below shows examples of crRNA for in vitro assays.

TABLE 6

| | SEQ ID | | SEQ ID | |
|---|---|---|---|---|
| Name | NO | DR sequence | NO | Spacer sequence |
| SSRNA | 111 | GGTTGGAAAGCCGGTTTTCTT | 112 | CGAGTAGATTGCTGTTCTacC |
| crRNA 5 | | TGATGTCACGGAAC | | AAGTAATCCA |
| SSRNA | 113 | GGTTGGAAAGCCGGTTTTCTT | 114 | TATGCTTCCGGCTCGTATGT |
| crRNA 1 | | TGATGTCACGGAAC | | TGTGTGGAATT |
| SSRNA | 115 | GGTTGGAAAGCCGGTTTTCTT | 116 | AACACAGGAAACAGCTATG |
| crRNA 2 | | TGATGTCACGGAAC | | ACCATGATTACG |
| SSRNA | 117 | GGTTGGAAAGCCGGTTTTCTT | 118 | AATTGTGAGCGGATAAACA |
| crRNA 3 | | TGATGTCACGGAAC | | CAGGAAACAGCT |
| SSRNA | 119 | GGTTGGAAAGCCGGTTTTCTT | 120 | TTGCTGTTCTacCAAGTAATC |
| crRNA 4 | | TGATGTCACGGAAC | | CATATTTCTA |
| SSRNA | 121 | GGTTGGAAAGCCGGTTTTCTT | 122 | TTTCTAGAGGATCCCCGGGT |
| crRNA 6 | | TGATGTCACGGAAC | | ACCGAGCTCGA |
| non targeting | 123 | GGTTGGAAAGCCGGTTTTCTT | 124 | CGCCAATATAACAATACAG |
| crRNA | | TGATGTCACGGAAC | | GTATTATAGTTG |
| MS2 guide 1 | 125 | GGTTGGAAAGCCGGTTTTCTT | 126 | CTTTGTTGTCTTCGACATGG |
| | | TGATGTCACGGAAC | | GTAATCCTCAT |
| MS2 guide 2 | 127 | GGTTGGAAAGCCGGTTTTCTT | 128 | CTTCTTTGTTGTCTTCGACA |
| | | TGATGTCACGGAAC | | TGGGTAATCCT |
| MS2 guide 3 | 129 | GGTTGGAAAGCCGGTTTTCTT | 130 | TAAAGAGTTGAACTTCTTTG |
| | | TGATGTCACGGAAC | | TTGTCTTCGAC |
| MS2 guide 4 | 131 | GGTTGGAAAGCCGGTTTTCTT | 132 | TCTTTGTTGTCTTCGACATG |
| | | TGATGTCACGGAAC | | GGTAATCCTCA |
| MS2 | 133 | GGTTGGAAAGCCGGTTTTCTT | 134 | TAATCCTCATGTTTGAATGG |
| additional | | TGATGTCACGGAAC | | CCGGCGTCTAT |
| spacer 1 | | | | |
| MS2 | 135 | GGTTGGAAAGCCGGTTTTCTT | 136 | TTCGACATGGGTAATCCTCA |
| additiona | | TGATGTCACGGAAC | | TGTTTGAATGG |
| spacer 2 | | | | |
| MS2 | 137 | GGTTGGAAAGCCGGTTTTCTT | 138 | CGAGGAAGATCAATACATA |
| additional | | TGATGTCACGGAAC | | AAGAGTTGAACT |
| spacer 3 | | | | |
| MS2 | 139 | GGTTGGAAAGCCGGTTTTCTT | 140 | TGGTAAATTTCGAGAGAAA |
| additional | | TGATGTCACGGAAC | | GATCGCGAGGAA |
| spacer 4 | | | | |
| MS2 | 141 | GGTTGGAAAGCCGGTTTTCTT | 142 | TTGAATGGCCGGCGTCTATT |
| additional | | TGATGTCACGGAAC | | AGTAGATGCCG |
| spacer 5 | | | | |
| fixed | 143 | GGTTGGAAAGCCGGTTTTCTT | 144 | AATCCATATTTCTAGAGGAT |
| position | | TGATGTCACGGAAC | | CCCCGGGTACC |
| SSRNA 1 | | | | |
| 150 nt guide | | | | |
| 1 | | | | |
| fixed | 145 | GGTTGGAAAGCCGGTTTTCTT | 146 | TGTTCTACCAAGTAATCCAT |
| position | | TGATGTCACGGAAC | | ATTTCTAGAGG |
| SSRNA 1 | | | | |
| 150 nt guide | | | | |
| 2 | | | | |
| fixed | 147 | GGTTGGAAAGCCGGTTTTCTT | 148 | TCGAGTAGATTGCTGTTCTA |
| position | | TGATGTCACGGAAC | | CCAAGTAATCC |
| SSRNA 1 | | | | |
| 150 nt guide | | | | |
| 3 | | | | |
| fixed | 149 | GGTTGGAAAGCCGGTTTTCTT | 150 | GCATGCCTGCAGGTCGAGT |
| position | | TGATGTCACGGAAC | | AGATTGCTGTTC |
| SSRNA 1 | | | | |
| 150nt guide | | | | |
| 4 | | | | |
| fixed | 151 | GGTTGGAAAGCCGGTTTTCTT | 152 | TTACGCCAAGCTTGCATGCC |
| position | | TGATGTCACGGAAC | | TGCAGGTCGAG |
| SSRNA 1 | | | | |
| 150 nt guide | | | | |
| 5 | | | | |
| fixed | 153 | GGTTGGAAAGCCGGTTTTCTT | 154 | GCTATGACCATGATTACGCC |
| position | | TGATGTCACGGAAC | | AAGCTTGCATG |
| SSRNA 1 | | | | |
| 150 nt guide | | | | |
| 6 | | | | |
| fixed | 155 | GGTTGGAAAGCCGGTTTTCTT | 156 | AATCCTCATGTTTGAATGGC |
| position MS2 | | TGATGTCACGGAAC | | CGGCGTCTATT |
| guide 1 | | | | |
| fixed | 157 | GGTTGGAAAGCCGGTTTTCTT | 158 | CTTCGACATGGGTAATCCTC |
| position MS2 | | TGATGTCACGGAAC | | ATGTTTGAATG |
| guide 2 | | | | |
| fixed | 159 | GGTTGGAAAGCCGGTTTTCTT | 160 | ACTTCTTTGTTGTCTTCGAC |
| position MS2 | | TGATGTCACGGAAC | | ATGGGTAATCC |
| guide 3 | | | | |
| fixed | 161 | GGTTGGAAAGCCGGTTTTCTT | 162 | CATAAAGAGTTGAACTTCTT |
| position MS2 | | TGATGTCACGGAAC | | TGTTGTCTTCG |
| guide 4 | | | | |
| fixed | 163 | GGTTGGAAAGCCGGTTTTCTT | 164 | AGGAAGATCAATACATAAA |
| position MS2 | | TGATGTCACGGAAC | | GAGTTGAACTTC |
| guide 5 | | | | |
| fixed | 165 | GGTTGGAAAGCCGGTTTTCTT | 166 | GAGAAAGATCGCGAGGAAG |
| position MS2 | | TGATGTCACGGAAC | | ATCAATACATAA |
| guide 6 | | | | |
| EGFP guide | 167 | GGTTGGAAAGCCGGTTTTCTT | 168 | AGGGTGCCGGTGATGCGGC |
| 1 | | TGATGTCACGGAAC | | ACTCGATCTTCA |
| EGFP guide | 169 | GGTTGGAAAGCCGGTTTTCTT | 170 | CCAGCTCGAACTCCACGCC |
| 2 | | TGATGTCACGGAAC | | GTTCAGGGTGCC |
| EGFP guide | 171 | GGTTGGAAAGCCGGTTTTCTT | 172 | CCCTCTCCGCCGCCCACCAG |
| 3 | | TGATGTCACGGAAC | | CTCGAACTCCA |
| EGFP guide | 173 | GGTTGGAAAGCCGGTTTTCTT | 174 | CGGCCCTGCTCGGGGGTGC |
| 4 | | TGATGTCACGGAAC | | CCTCTCCGCCGC |
| EGFP guide | 175 | GGTTGGAAAGCCGGTTTTCTT | 176 | ACGTGGCTCAGCAGGTAGG |
| 5 | | TGATGTCACGGAAC | | GGCTGAAGGTCA |
| EGFP guide | 177 | GGTTGGAAAGCCGGTTTTCTT | 178 | AGGTGCCGAAGTGGTAGAA |
| 6 | | TGATGTCACGGAAC | | GCCGTAGCCCAT |
| EGFP guide | 179 | GGTTGGAAAGCCGGTTTTCTT | 180 | TGGCGTGCAGGAAGGGGTT |
| 7 | | TGATGTCACGGAAC | | CTCGTAGCCGCT |
| EGFP guide | 181 | GGTTGGAAAGCCGGTTTTCTT | 182 | GTACTTCTCGATGCGGGTGT |
| 8 | | TGATGTCACGGAAC | | TGGTGTAGCCG |
| EGFP guide | 183 | GGTTGGAAAGCCGGTTTTCTT | 184 | GCGGCCGGCCTCGTAGCGG |
| 9 | | TGATGTCACGGAAC | | TAGCTGAAGCTC |
| EGFP guide | 185 | GGTTGGAAAGCCGGTTTTCTT | 186 | AAGCCGGTGCCCACCACCTT |
| 10 | | TGATGTCACGGAAC | | GAAGTCGCCGA |
| EGFP guide | 187 | GGTTGGAAAGCCGGTTTTCTT | 188 | AAGATCACGCTGTCCTCGG |
| 11 | | TGATGTCACGGAAC | | GGAAGCCGGTGC |
| GwCas7-11c | 189 | GTTTGGAAGCCGGGTCGAAG | 190 | CTTTGTTGTCTTCGACATGG |
| MS2 crRNA | | TCAGGCCCGTTAAGAC | | GTAATCCTCAT |
| CjcCas7-11b | 191 | cttgaagactaaaggaaggaa | 192 | CTTTGTTGTCTTCGACATGG |
| MS2 crRNA | | ttaatgtcacggtac | | GTAATCCTCAT |
| GwCas7-11c | 193 | GTTTGGAAGCCGGGTCGAAG | 194 | CGCCAATATAACAATACAG |
| non-targeting | | TCAGGCCCGTTAAGAC | | GTATTATAGTTG |
| crRNA | | | | |
| CjcCas7-11b | 195 | cttgaagactaaaggaaggaa | 196 | CGCCAATATAACAATACAG |
| non-targeting | | ttaatgtcacggtac | | GTATTATAGTTG |
| crRNA | | | | |
| MS2 crRNA | 197 | GTTGGAAAGCCGGTTTTCTTT | 198 | CTTTGTTGTCTTCGACATGG |
| DR length | | GATGTCACGGAAC | | GTAATCCTCAT |
| MS2 crRNA | 199 | GGAAAGCCGGTTTTCTTTGAT | 200 | CTTTGTTGTCTTCGACATGG |
| DR length | | GTCACGGAAC | | GTAATCCTCAT |
| MS2 crRNA | 201 | AAGCCGGTTTTCTTTGATGTC | 202 | CTTTGTTGTCTTCGACATGG |
| DR length | | ACGGAAC | | GTAATCCTCAT |
| MS2 crRNA | 203 | CCGGTTTTCTTTGATGTCACG | 204 | CTTTGTTGTCTTCGACATGG |
| DR length | | GAAC | | GTAATCCTCAT |
| MS2 crRNA | 205 | GTTTTCTTTGATGTCACGGAA | 206 | CTTTGTTGTCTTCGACATGG |
| DR length | | C | | GTAATCCTCAT |
| MS2 crRNA | 207 | TCTTTGATGTCACGGAAC | 208 | CTTTGTTGTCTTCGACATGG |
| DR length | | | | GTAATCCTCAT |
| MS2 crRNA | 209 | TTGATGTCACGGAAC | 210 | CTTTGTTGTCTTCGACATGG |
| DR length | | | | GTAATCCTCAT |
| MS2 crRNA | 211 | ATGTCACGGAAC | 212 | CTTTGTTGTCTTCGACATGG |
| DR length | | | | GTAATCCTCAT |
| MS2 crRNA | 213 | TCACGGAAC | 214 | CTTTGTTGTCTTCGACATGG |
| DR length | | | | GTAATCCTCAT |
| MS2 crRNA | 215 | CGGAAC | 216 | CTTTGTTGTCTTCGACATGG |
| DR length | | | | GTAATCCTCAT |
| MS2 crRNA | 217 | GGTTGGAAAGCCGGTTTTCTT | 218 | CTTTGTTGTCTTCGACATGG |
| spacer length | | TGATGTCACGGAAC | | GTAATCCTCA |
| MS2 crRNA | 219 | GGTTGGAAAGCCGGTTTTCTT | 220 | CTTTGTTGTCTTCGACATGG |
| spacer length | | TGATGTCACGGAAC | | GTAATCCT |
| MS2 crRNA | 221 | GGTTGGAAAGCCGGTTTTCTT | 222 | CTTTGTTGTCTTCGACATGG |
| spacer length | | TGATGTCACGGAAC | | GTAAT |
| MS2 crRNA | 223 | GGTTGGAAAGCCGGTTTTCTT | 224 | CTTTGTTGTCTTCGACATGG |
| spacer length | | TGATGTCACGGAAC | | GT |
| MS2 crRNA | 225 | GGTTGGAAAGCCGGTTTTCTT | 226 | CTTTGTTGTCTTCGACATG |
| spacer length | | TGATGTCACGGAAC | | |
| MS2 crRNA | 227 | GGTTGGAAAGCCGGTTTTCTT | 228 | CTTTGTTGTCTTCGAC |
| spacer length | | TGATGTCACGGAAC | | |
| MS2 crRNA | 229 | GGTTGGAAAGCCGGTTTTCTT | 230 | CTTTGTTGTCTTC |
| spacer length | | TGATGTCACGGAAC | | |
| MS2 crRNA | 231 | GGTTGGAAAGCCGGTTTTCTT | 232 | CTTTGTTGTC |
| spacer length | | TGATGTCACGGAAC | | |
| MS2 crRNA | 233 | TTGATGTCACGGAAC | 234 | CTTTGTTGTCTTCGACATGG |
| 1 DR WT | | | | GTAATCCTCAT |
| MS2 crRNA | 235 | ATGATGTCACGGAAC | 236 | CTTTGTTGTCTTCGACATGG |
| 1 DR mutant | | | | GTAATCCTCAT |
| 1 | | | | |
| MS2 crRNA | 237 | TAGATGTCACGGAAC | 238 | CTTTGTTGTCTTCGACATGG |
| 1 DR mutant | | | | GTAATCCTCAT |
| 2 | | | | |
| MS2 crRNA | 239 | TTCATGTCACGGAAC | 240 | CTTTGTTGTCTTCGACATGG |
| 1 DR mutant | | | | GTAATCCTCAT |
| 3 | | | | |
| MS2 crRNA | 241 | TTGTTGTCACGGAAC | 242 | CTTTGTTGTCTTCGACATGG |
| 1 DR mutant | | | | GTAATCCTCAT |
| 4 | | | | |
| MS2 crRNA | 243 | TTGAAGTCACGGAAC | 244 | CTTTGTTGTCTTCGACATGG |
| 1 DR mutant | | | | GTAATCCTCAT |
| 5 | | | | |
| MS2 crRNA | 245 | TTGATCTCACGGAAC | 246 | CTTTGTTGTCTTCGACATGG |
| 1 DR mutant | | | | GTAATCCTCAT |
| 6 | | | | |
| MS2 crRNA | 247 | TTGATGACACGGAAC | 248 | CTTTGTTGTCTTCGACATGG |
| 1 DR mutant | | | | GTAATCCTCAT |
| 7 | | | | |
| MS2 crRNA | 249 | TTGATGTGACGGAAC | 250 | CTTTGTTGTCTTCGACATGG |
| 1 DR mutant | | | | GTAATCCTCAT |
| 8 | | | | |
| MS2 crRNA | 251 | TTGATGTCTCGGAAC | 252 | CTTTGTTGTCTTCGACATGG |
| 1 DR mutant | | | | GTAATCCTCAT |
| 9 | | | | |
| MS2 crRNA | 253 | TTGATGTCAGGGAAC | 254 | CTTTGTTGTCTTCGACATGG |
| 1 DR mutant | | | | GTAATCCTCAT |
| 10 | | | | |
| MS2 crRNA | 255 | TTGATGTCACCGAAC | 256 | CTTTGTTGTCTTCGACATGG |
| 1 DR mutant | | | | GTAATCCTCAT |
| 11 | | | | |
| MS2 crRNA | 257 | TTGATGTCACGCAAC | 258 | CTTTGTTGTCTTCGACATGG |
| 1 DR mutant | | | | GTAATCCTCAT |
| 12 | | | | |
| MS2 crRNA | 259 | TTGATGTCACGGTAC | 260 | CTTTGTTGTCTTCGACATGG |
| 1 DR mutant | | | | GTAATCCTCAT |
| 13 | | | | |
| MS2 crRNA | 261 | TTGATGTCACGGATC | 262 | CTTTGTTGTCTTCGACATGG |
| 1 DR mutant | | | | GTAATCCTCAT |
| 14 | | | | |
| MS2 crRNA | 263 | TTGATGTCACGGAAG | 264 | CTTTGTTGTCTTCGACATGG |
| 1 DR mutant | | | | GTAATCCTCAT |
| 15 | | | | |
| MS2 crRNA | 265 | TTGATGTCACGGAAC | 266 | CTTTGTTGTCTTCGACATGG |
| 1 DR WT | | | | GTAATCCTCAT |
| MS2 crRNA | 267 | AAGATGTCACGGAAC | 268 | CTTTGTTGTCTTCGACATGG |
| 1 DR double | | | | GTAATCCTCAT |
| mutant 1 | | | | |
| MS2 crRNA | 269 | TACATGTCACGGAAC | 270 | CTTTGTTGTCTTCGACATGG |
| 1 DR double | | | | GTAATCCTCAT |
| mutant 2 | | | | |
| MS2 crRNA | 271 | TTCTTGTCACGGAAC | 272 | CTTTGTTGTCTTCGACATGG |
| 1 DR double | | | | GTAATCCTCAT |
| mutant 3 | | | | |
| MS2 crRNA | 273 | TTGTAGTCACGGAAC | 274 | CTTTGTTGTCTTCGACATGG |
| 1 DR double | | | | GTAATCCTCAT |
| mutant 4 | | | | |
| MS2 crRNA | 275 | TTGAACTCACGGAAC | 276 | CTTTGTTGTCTTCGACATGG |
| 1 DR double | | | | GTAATCCTCAT |
| mutant 5 | | | | |
| MS2 crRNA | 277 | TTGATCACACGGAAC | 278 | CTTTGTTGTCTTCGACATGG |
| 1 DR double | | | | GTAATCCTCAT |
| mutant 6 | | | | |
| MS2 crRNA | 279 | TTGATGAGACGGAAC | 280 | CTTTGTTGTCTTCGACATGG |
| 1 DR double | | | | GTAATCCTCAT |
| mutant 7 | | | | |
| MS2 crRNA | 281 | TTGATGTGTCGGAAC | 282 | CTTTGTTGTCTTCGACATGG |
| 1 DR double | | | | GTAATCCTCAT |
| mutant 8 | | | | |
| MS2 crRNA | 283 | TTGATGTCTGGGAAC | 284 | CTTTGTTGTCTTCGACATGG |
| 1 DR double | | | | GTAATCCTCAT |
| mutant 9 | | | | |
| MS2 crRNA | 285 | TTGATGTCAGCGAAC | 286 | CTTTGTTGTCTTCGACATGG |
| 1 DR double | | | | GTAATCCTCAT |
| mutant 10 | | | | |
| MS2 crRNA | 287 | TTGATGTCACCCAAC | 288 | CTTTGTTGTCTTCGACATGG |
| 1 DR double | | | | GTAATCCTCAT |
| mutant 11 | | | | |
| MS2 crRNA | 289 | TTGATGTCACGCTAC | 290 | CTTTGTTGTCTTCGACATGG |
| 1 DR double | | | | GTAATCCTCAT |
| mutant 12 | | | | |
| MS2 crRNA | 291 | TTGATGTCACGGTTC | 292 | CTTTGTTGTCTTCGACATGG |
| 1 DR double | | | | GTAATCCTCAT |
| mutant 13 | | | | |
| MS2 crRNA | 293 | TTGATGTCACGGATG | 294 | CTTTGTTGTCTTCGACATGG |
| 1 DR double | | | | GTAATCCTCAT |
| mutant 14 | | | | |
| COVID-19 N | 295 | GGTTGGAAAGCCGGTTTTCTT | 296 | ACCAAACGTAATGCGGGGT |
| gene crRNA | | TGATGTCACGGAAC | | GCATTTCGCTGA |
| 1 | | | | |
| COVID-19 N | 297 | GGTTGGAAAGCCGGTTTTCTT | 298 | GTTGAATCTGAGGGTCCACC |
| gene crRNA | | TGATGTCACGGAAC | | AAACGTAATGC |
| 2 | | | | |
| COVID-19 N | 299 | GGTTGGAAAGCCGGTTTTCTT | 300 | CATTCTGGTTACTGCCAGTT |
| gene crRNA | | TGATGTCACGGAAC | | GAATCTGAGGG |
| 3 | | | | |
| COVID-19 N | 301 | GGTTGGAAAGCCGGTTTTCTT | 302 | GCCCCACTGCGTTCTCCATT |
| gene crRNA | | TGATGTCACGGAAC | | CTGGTTACTGC |
| 4 | | | | |
| COVID-19 N | 303 | GGTTGGAAAGCCGGTTTTCTT | 304 | ACGTTGTTTTGATCGCGCCC |
| gene crRNA | | TGATGTCACGGAAC | | CACTGCGTTCT |
| 5 | | | | |
| COVID-19 N | 305 | GGTTGGAAAGCCGGTTTTCTT | 306 | GGTAAACCTTGGGGCCGAC |
| gene crRNA | | TGATGTCACGGAAC | | GTTGTTTTGATC |
| 6 | | | | |
| COVID-19 N | 307 | GGTTGGAAAGCCGGTTTTCTT | 308 | AGACGCAGTATTATTGGGT |
| gene crRNA | | TGATGTCACGGAAC | | AAACCTTGGGGC |
| 7 | | | | |
| COVID-19 N | 309 | GGTTGGAAAGCCGGTTTTCTT | 310 | TGAGAGCGGTGAACCAAGA |
| gene crRNA | | TGATGTCACGGAAC | | CGCAGTATTATT |
| 8 | | | | |
| Influenza A | 311 | GGTTGGAAAGCCGGTTTTCTT | 312 | CCGGGAAGTTTTTGAGCGA |
| segment 4 | | TGATGTCACGGAAC | | AAACCAGACATA |
| HA gene | | | | |
| crRNA 1 | | | | |
| Influenza A | 313 | GGTTGGAAAGCCGGTTTTCTT | 314 | CGTGCTGTTGTCATTTCCGG |
| segment 4 | | TGATGTCACGGAAC | | GAAGTTTTTGA |
| HA gene | | | | |
| crRNA 2 | | | | |
| Influenza A | 315 | GGTTGGAAAGCCGGTTTTCTT | 316 | CAAGGCACAGCGTTGCCGT |
| segment 4 | | TGATGTCACGGAAC | | GCTGTTGTCATT |
| HA gene | | | | |
| crRNA 3 | | | | |
| Influenza A | 317 | GGTTGGAAAGCCGGTTTTCTT | 318 | GGTACTGCATGGTGCCCAA |
| segment 4 | | TGATGTCACGGAAC | | GGCACAGCGTTG |
| HA gene | | | | |
| crRNA 4 | | | | |
| Influenza A | 319 | GGTTGGAAAGCCGGTTTTCTT | 320 | TCACTATCGTTCCGTTTGGT |
| segment 4 | | TGATGTCACGGAAC | | ACTGCATGGTG |
| HA gene | | | | |
| crRNA 5 | | | | |
| Influenza A | 321 | GGTTGGAAAGCCGGTTTTCTT | 322 | GTCATTCGTGATTGTTTTCA |
| segment 4 | | TGATGTCACGGAAC | | CTATCGTTCCG |
| HA gene | | | | |
| crRNA 6 | | | | |
| Influenza A | 323 | GGTTGGAAAGCCGGTTTTCTT | 324 | AGTGACTTCAATTTGGTCAT |
| segment 4 | | TGATGTCACGGAAC | | TCGTGATTGTT |
| HA gene | | | | |
| crRNA 7 | | | | |
| Influenza A | 325 | GGTTGGAAAGCCGGTTTTCTT | 326 | ACCAGTTCAGTAGCATTAGT |
| segment 4 | | TGATGTCACGGAAC | | GACTTCAATTT |
| HA gene | | | | |
| crRNA 8 | | | | |
| EGFP guide | 327 | GGTTGGAAAGCCGGTTTTCTT | 328 | TGGGGTGCAGGTGCTCCAC |
| 12 | | TGATGTCACGGAAC | | GGTGGCGTTGCT |
| EGFP guide | 329 | GGTTGGAAAGCCGGTTTTCTT | 330 | AAGGTGCGGGCGAAGCTGC |
| 13 | | TGATGTCACGGAAC | | CCACCAGCACGT |
| EGFP guide | 331 | GGTTGGAAAGCCGGTTTTCTT | 332 | GTCCACCACGAAGCTGTAG |
| 14 | | TGATGTCACGGAAC | | TAGCCGCCGTCG |
| EGFP guide | 333 | GGTTGGAAAGCCGGTTTTCTT | 334 | TGCTGGGGTGGATGGCGCT |
| 15 | | TGATGTCACGGAAC | | CTTGAAGTGCAT |
| EGFP guide | 335 | GGTTGGAAAGCCGGTTTTCTT | 336 | GGAAGGCGAACATGGGGCC |
| 16 | | TGATGTCACGGAAC | | CCCGTTCTGCAG |
| MS2 tiling | 337 | GGTTGGAAAGCCGGTTTTCTT | 338 | AATCCTCATGTTTGAATGGC |
| guide 1 | | TGATGTCACGGAAC | | CGGCGTCTATT |
| MS2 tiling | 339 | GGTTGGAAAGCCGGTTTTCTT | 340 | GGTAATCCTCATGTTTGAAT |
| guide 2 | | TGATGTCACGGAAC | | GGCCGGCGTCT |
| MS2 tiling | 341 | GGTTGGAAAGCCGGTTTTCTT | 342 | ATGGGTAATCCTCATGTTTG |
| guide 3 | | TGATGTCACGGAAC | | AATGGCCGGCG |
| MS2 tiling | 343 | GGTTGGAAAGCCGGTTTTCTT | 344 | GACATGGGTAATCCTCATGT |
| guide 4 | | TGATGTCACGGAAC | | TTGAATGGCCG |
| MS2 tiling | 345 | GGTTGGAAAGCCGGTTTTCTT | 346 | TTCGACATGGGTAATCCTCA |
| guide 5 | | TGATGTCACGGAAC | | TGTTTGAATGG |
| MS2 tiling | 347 | GGTTGGAAAGCCGGTTTTCTT | 348 | GTCTTCGACATGGGTAATCC |
| guide 6 | | TGATGTCACGGAAC | | TCATGTTTGAA |
| MS2 tiling | 349 | GGTTGGAAAGCCGGTTTTCTT | 350 | GTTGTCTTCGACATGGGTAA |
| guide 7 | | TGATGTCACGGAAC | | TCCTCATGTTT |
| MS2 tiling | 351 | GGTTGGAAAGCCGGTTTTCTT | 352 | TTTGTTGTCTTCGACATGGG |
| guide 8 | | TGATGTCACGGAAC | | TAATCCTCATG |
| MS2 tiling | 353 | GGTTGGAAAGCCGGTTTTCTT | 354 | TTCTTTGTTGTCTTCGACAT |
| guide 9 | | TGATGTCACGGAAC | | GGGTAATCCTC |
| MS2 tiling | 355 | GGTTGGAAAGCCGGTTTTCTT | 356 | AACTTCTTTGTTGTCTTCGA |
| guide 10 | | TGATGTCACGGAAC | | CATGGGTAATC |
| Cjc MS2 | 357 | CTTGAAGACTAAAGGAAGGA | 358 | AATCCTCATGTTTGAATGGC |
| 60 bp ssRNA | | ATTAATGTCACGGTAC | | CGGCGTCTATT |
| crRNA 1 | | | | |
| Cjc MS2 | 359 | CTTGAAGACTAAAGGAAGGA | 360 | GGTAATCCTCATGTTTGAAT |
| 60 bp ssRNA | | ATTAATGTCACGGTAC | | GGCCGGCGTCT |
| crRNA 2 | | | | |
| Cjc MS2 | 361 | CTTGAAGACTAAAGGAAGGA | 362 | ATGGGTAATCCTCATGTTTG |
| 60 bp ssRNA | | ATTAATGTCACGGTAC | | AATGGCCGGCG |
| crRNA 3 | | | | |
| Cjc MS2 | 363 | CTTGAAGACTAAAGGAAGGA | 364 | GACATGGGTAATCCTCATGT |
| 60 bp ssRNA | | ATTAATGTCACGGTAC | | TTGAATGGCCG |
| crRNA 4 | | | | |
| Cjc MS2 | 365 | CTTGAAGACTAAAGGAAGGA | 366 | TTCGACATGGGTAATCCTCA |
| 60 bp ssRNA | | ATTAATGTCACGGTAC | | TGTTTGAATGG |
| crRNA 5 | | | | |
| Cjc MS2 | 367 | CTTGAAGACTAAAGGAAGGA | 368 | GTCTTCGACATGGGTAATCC |
| 60 bp ssRNA | | ATTAATGTCACGGTAC | | TCATGTTTGAA |
| crRNA 6 | | | | |
| Cjc MS2 | 369 | CTTGAAGACTAAAGGAAGGA | 370 | GTTGTCTTCGACATGGGTAA |
| 60 bp ssRNA | | ATTAATGTCACGGTAC | | TCCTCATGTTT |
| crRNA 7 | | | | |
| Cjc MS2 | 371 | CTTGAAGACTAAAGGAAGGA | 372 | TTTGTTGTCTTCGACATGGG |
| 60 bp ssRNA | | ATTAATGTCACGGTAC | | TAATCCTCATG |
| crRNA 8 | | | | |
| Cjc MS2 | 373 | CTTGAAGACTAAAGGAAGGA | 374 | TTCTTTGTTGTCTTCGACAT |
| 60 bp ssRNA | | ATTAATGTCACGGTAC | | GGGTAATCCTC |
| crRNA 9 | | | | |
| Cjc MS2 | 375 | CTTGAAGACTAAAGGAAGGA | 376 | AACTTCTTTGTTGTCTTCGA |
| 60 bp ssRNA | | ATTAATGTCACGGTAC | | CATGGGTAATC |
| crRNA 10 | | | | |
| Cjc MS2 | 377 | CTTGAAGACTAAAGGAAGGA | 378 | TTGAACTTCTTTGTTGTCTTC |
| 60 bp ssRNA | | ATTAATGTCACGGTAC | | GACATGGGTA |
| crRNA 11 | | | | |
| Gw MS2 | 379 | GTTTGGAAGCCGGGTCGAAG | 380 | AAACGTAATGCGGGGTGCA |
| 60 bp ssRNA | | TCAGGCCCGTTAAGAC | | TTTCGCTGATTT |
| crRNA 1 | | | | |
| Gw MS2 | 381 | GTTTGGAAGCCGGGTCGAAG | 382 | ACCAAACGTAATGCGGGGT |
| 60 bp ssRNA | | TCAGGCCCGTTAAGAC | | GCATTTCGCTGA |
| crRNA 2 | | | | |
| Gw MS2 | 383 | GTTTGGAAGCCGGGTCGAAG | 384 | TCCACCAAACGTAATGCGG |
| 60 bp ssRNA | | TCAGGCCCGTTAAGAC | | GGTGCATTTCGC |
| crRNA 3 | | | | |
| Gw MS2 | 385 | GTTTGGAAGCCGGGTCGAAG | 386 | GGGTCCACCAAACGTAATG |
| 60 bp ssRNA | | TCAGGCCCGTTAAGAC | | CGGGGTGCATTT |
| crRNA 4 | | | | |
| Gw MS2 | 387 | GTTTGGAAGCCGGGTCGAAG | 388 | TGAGGGTCCACCAAACGTA |
| 60 bp ssRNA | | TCAGGCCCGTTAAGAC | | ATGCGGGGTGCA |
| crRNA 5 | | | | |
| Gw MS2 | 389 | GTTTGGAAGCCGGGTCGAAG | 390 | ATCTGAGGGTCCACCAAAC |
| 60 bp ssRNA | | TCAGGCCCGTTAAGAC | | GTAATGCGGGGT |
| crRNA 6 | | | | |
| Gw MS2 | 391 | GTTTGGAAGCCGGGTCGAAG | 392 | TGAATCTGAGGGTCCACCA |
| 60 bp ssRNA | | TCAGGCCCGTTAAGAC | | AACGTAATGCGG |
| crRNA 7 | | | | |
| Gw MS2 | 393 | GTTTGGAAGCCGGGTCGAAG | 394 | AGTTGAATCTGAGGGTCCA |
| 60 bp ssRNA | | TCAGGCCCGTTAAGAC | | CCAAACGTAATG |
| crRNA 8 | | | | |
| Gw MS2 | 395 | GTTTGGAAGCCGGGTCGAAG | 396 | GCCAGTTGAATCTGAGGGT |
| 60 bp ssRNA | | TCAGGCCCGTTAAGAC | | CCACCAAACGTA |
| crRNA 9 | | | | |
| Gw MS2 | 397 | GTTTGGAAGCCGGGTCGAAG | 398 | ACTGCCAGTTGAATCTGAG |
| 60 bp ssRNA | | TCAGGCCCGTTAAGAC | | GGTCCACCAAAC |
| crRNA 10 | | | | |
| Gw MS2 | 399 | GTTTGGAAGCCGGGTCGAAG | 400 | GTTACTGCCAGTTGAATCTG |
| 60 bp ssRNA | | TCAGGCCCGTTAAGAC | | AGGGTCCACCA |
| crRNA 11 | | | | |

Table 7 below shows examples of mammalian plasmids.

TABLE 7

| Plasmid | | |
|---|---|---|
| number | Name | Benchling link |
| pDF0158 | pCMV-huDsiGRAMP N term | https://benchling.com/s/seq- |
| | msfGFP mammalian expression | 16V8YUC36yaVMw4GYawz |
| pDF0159 | pCMV-huDsiGRAMP mammalian | https://benchling.com/s/seq- |
| | expression | V4NVwiioQSHLp6fkKY5I |
| pDF0160 | pCMV-huDsiGRAMP C term | https://benchling.com/s/seq- |
| | msfGFP mammalian expression | 6KYYZvPGjqOWNIPwLN2u |
| pDF0113 | pU6-Eco31i-Eco31i-DsiCas7-11a full DR | https://benchling.com/s/seq- |
| | guide scaffold with golden gate site | fl07Vm8NmTi2ydJnbZGS |
| pDF0114 | pU6-Eco31i-Eco31i-DsiCas7-11a mature DR | https://benchling.com/s/seq- |
| | guide scaffold with golden gate site | 22EWyWJAvvfpX2EsdJxs |
| pDF0111 | CMV-Cluciferase(STOP85)-polyA EF1a- | https://benchling.com/s/seq- |
| | G-luciferase-polyA (pAB0040) | 9fEQxFBZrIEQo8Ta407A |
| pDF0112 | CMV-Cluciferase-polyA | https://benchling.com/s/seq- |
| | EF1a-G-luciferase-polyA | QDT25JIo4luxluesN196 |
| pDF0234 | pCMV-huDsiGRAMP | https://benchling.com/s/seq- |
| | (5′ UTR from BE constructs) dead mutant | cNh21gwflmDV66VanySf |
| pDF0237 | pCMV-dead huDsiGRAMP-NES-huADAR2 | https://benchling.com/s/seq- |
| | | OKxBeGoAOWuk6GbgfAa2 |
| pDF0228 | pCMV-huDsiGRAMP-NES-huADAR2 | https://benchling.com/s/seq- |
| | | BnKsJ4pko0RcknikJMlr |

Table 8 below shows examples of Cas7-11 guides for mammalian.

TABLE 8

| SEQ | | | | Application |
|---|---|---|---|---|
| ID | | | | (Knockdown/ |
| NO | Name | Spacer sequence | Target | editing) |
| 401 | DsiGRAMP mammalian Gluc | GCAGATCAGGGCAAACAG | Gluc | Knockdown |
| | guide 1 F | AACTTTGACTCCC | | |
| 402 | DsiGRAMP mammalian Gluc | CGATGTTGAAGTCTTCGTT | Gluc | Knockdown |
| | guide 2 F | GTTCTCGGTGGG | | |
| 403 | DsiGRAMP mammalian Gluc | GCAACTTCCCGCGGTCAGC | Gluc | Knockdown |
| | guide 3 F | ATCGAGATCCGT | | |
| 404 | DsiGRAMP mammalian Gluc | TGCAGCCAGCTTTCCGGGC | Gluc | Knockdown |
| | guide 4 F | ATTGGCTTCCAT | | |
| 405 | DsiGRAMP mammalian Gluc | TGAACTTCTTCATCTTGGGC | Gluc | Knockdown |
| | guide 5 F | GTGCACTTGAT | | |
| 406 | DsiGRAMP mammalian Gluc | GAATGTCGACGATCGCCTC | Gluc | Knockdown |
| | guide 6 F | GCCTATGCCGCC | | |
| 407 | DsiGRAMP mammalian Gluc | ATGAACTGCTCCATGGGCT | Gluc | Knockdown |
| | guide 7 F | CCAAGTCCTTGA | | |
| 408 | DsiGRAMP mammalian Gluc | GCAGCCAGTTGTGCAGTCC | Gluc | Knockdown |
| | guide 8 F | ACACACAGATCG | | |
| 409 | DsiGRAMP mammalian Gluc | GCGTTGCGGCAGCCACTTC | Gluc | Knockdown |
| | guide 9 F | TTGAGCAGGTCA | | |
| 410 | DsiGRAMP mammalian Gluc | TGTCCACCTGGCCCTGGAT | Gluc | Knockdown |
| | guide 10 F | CTTGCTGGCAAA | | |
| 411 | DsiGRAMP mammalian Non- | GGTAATGCCTGGCTTGTCG | Gluc | Knockdown |
| | targeting 1 F | ACGCATAGTCTG | | |
| 412 | DsiGRAMP mammalian Non- | GGGAACCTTGGCCGTTATA | Gluc | Knockdown |
| | targeting 2 F | AAGTCTGACCAG | | |
| 413 | KRAS guide 1 | TATAATGGTGAATATCTTC | KRAS | Knockdown |
| | | AAATGATTTAGT | | |
| 414 | KRAS guide 2 | ATGTATAGAAGGCATCATC | KRAS | Knockdown |
| | | AACACCCTGTCT | | |
| 415 | KRAS guide 3 | GGTTAAAAATTTACAGATT | KRAS | Knockdown |
| | | GTGCTGAGCTTG | | |
| 416 | PPIB guide 1 | GTAGATGCTCTTTCCTCCTG | PPIB | Knockdown |
| | | TGCCATCTCCC | | |
| 417 | PPIB guide 2 | CAGTTTGAAGTTCTCATCG | PPIB | Knockdown |
| | | GGGAAGCGCTCA | | |
| 418 | PPIB guide 3 | CAGTGTTGGTAGGAGTTTG | PPIB | Knockdown |
| | | TTACAAAAGTGA | | |
| 419 | MALAT1 guide 1 | CTTGGCCAAGTCTGTTATGT | MALA | Knockdown |
| | | TCACCTGAAAA | T1 | |
| 420 | MALAT1 guide 2 | CAAAATGTACTCAGCTTCA | MALA | Knockdown |
| | | ATCACAAATACG | T1 | |
| 421 | MALAT1 guide 3 | GGTTATAGCTTGACAAGCA | MALA | Knockdown |
| | | ATTAACTTTAAA | T1 | |
| 422 | CXCR4 guide 1 | ATGATAATGCAATAGCAGG | CXCR4 | Knockdown |
| | | ACAGGATGACAA | | |
| 423 | Non-targeting 1 | GGTAATGCCTGGCTTGTCG | — | Knockdown |
| | | ACGCATAGTCTG | | |
| 424 | Non-targeting 2 | GGGAACCTTGGCCGTTATA | — | Knockdown |
| | | AAGTCTGACCAG | | |
| 425 | Cluc W85X guide MM1 | CATCCTGCGGCCTCTACTCT | Cluc | Editing |
| | | GCATTCAATTACATACTGA | | |
| | | CACATTCGGCA | | |
| 426 | Cluc W85X guide MM3 | ACCATCCTGCGGCCTCTAC | Cluc | Editing |
| | | TCTGCATTCAATTACATACT | | |
| | | GACACATTCGG | | |
| 427 | Cluc W85X guide MM5 | AAACCATCCTGCGGCCTCT | Cluc | Editing |
| | | ACTCTGCATTCAATTACAT | | |
| | | ACTGACACATTC | | |
| 428 | Cluc W85X guide MM7 | CTAAACCATCCTGCGGCCT | Cluc | Editing |
| | | CTACTCTGCATTCAATTACA | | |
| | | TACTGACACAT | | |
| 429 | Cluc W85X guide MM9 | TTCTAAACCATCCTGCGGC | Cluc | Editing |
| | | CTCTACTCTGCATTCAATTA | | |
| | | CATACTGACAC | | |
| 430 | Cluc W85X guide MM11 | TGTTCTAAACCATCCTGCG | Cluc | Editing |
| | | GCCTCTACTCTGCATTCAAT | | |
| | | TACATACTGAC | | |
| 431 | Cluc W85X guide MM13 | AATGTTCTAAACCATCCTG | Cluc | Editing |
| | | CGGCCTCTACTCTGCATTCA | | |
| | | ATTACATACTG | | |
| 432 | Cluc W85X guide MM15 | AGAATGTTCTAAACCATCC | Cluc | Editing |
| | | TGCGGCCTCTACTCTGCATT | | |
| | | CAATTACATAC | | |
| 433 | Cluc W85X guide MM17 | ATAGAATGTTCTAAACCAT | Cluc | Editing |
| | | CCTGCGGCCTCTACTCTGC | | |
| | | ATTCAATTACAT | | |
| 434 | Cluc W85X guide MM19 | CCATAGAATGTTCTAAACC | Cluc | Editing |
| | | ATCCTGCGGCCTCTACTCTG | | |
| | | CATTCAATTAC | | |
| 435 | Cluc W85X guide MM21 | TTCCATAGAATGTTCTAAA | Cluc | Editing |
| | | CCATCCTGCGGCCTCTACTC | | |
| | | TGCATTCAATT | | |
| 436 | Cluc W85X guide MM23 | CTTTCCATAGAATGTTCTAA | Cluc | Editing |
| | | ACCATCCTGCGGCCTCTAC | | |
| | | TCTGCATTCAA | | |
| 437 | Cluc W85X guide MM25 | CTCTTTCCATAGAATGTTCT | Cluc | Editing |
| | | AAACCATCCTGCGGCCTCT | | |
| | | ACTCTGCATTC | | |
| 438 | Cluc W85X guide MM27 | ATCTCTTTCCATAGAATGTT | Cluc | Editing |
| | | CTAAACCATCCTGCGGCCT | | |
| | | CTACTCTGCAT | | |
| 439 | Cluc W85X guide MM29 | GAATCTCTTTCCATAGAAT | Cluc | Editing |
| | | GTTCTAAACCATCCTGCGG | | |
| | | CCTCTACTCTGC | | |
| 440 | Cluc W85X guide MM31 | TGGAATCTCTTTCCATAGA | Cluc | Editing |
| | | ATGTTCTAAACCATCCTGC | | |
| | | GGCCTCTACTCT | | |
| 441 | Cluc W85X guide MM33 | ACTGGAATCTCTTTCCATA | Cluc | Editing |
| | | GAATGTTCTAAACCATCCT | | |
| | | GCGGCCTCTACT | | |
| 442 | Cluc W85X guide MM35 | GAACTGGAATCTCTTTCCA | Cluc | Editing |
| | | TAGAATGTTCTAAACCATC | | |
| | | CTGCGGCCTCTA | | |
| 443 | Cluc W85X guide MM37 | TGGAACTGGAATCTCTTTC | Cluc | Editing |
| | | CATAGAATGTTCTAAACCA | | |
| | | TCCTGCGGCCTC | | |
| 444 | Cluc W85X guide MM39 | CCTGGAACTGGAATCTCTT | Cluc | Editing |
| | | TCCATAGAATGTTCTAAAC | | |
| | | CATCCTGCGGCC | | |
| 445 | Cluc W85X guide MM41 | TTCCTGGAACTGGAATCTC | Cluc | Editing |
| | | TTTCCATAGAATGTTCTAA | | |
| | | ACCATCCTGCGG | | |
| 446 | Cluc W85X guide MM43 | GGTTCCTGGAACTGGAATC | Cluc | Editing |
| | | TCTTTCCATAGAATGTTCTA | | |
| | | AACCATCCTGC | | |
| 447 | LwaCas13a guide | ACCCAGGAATCTCAGGAAT | Gluc | Knockdown |
| | for comparison | GTCGACGAT | | |
| 448 | PspCas13b guide | GGGCATTGGCTTCCATCTCT | Gluc | Knockdown |
| | for comparison | TTGAGCACCT | | |
| 449 | RfxCas13d guide | ACCCAGGAATCTCAGGAAT | Gluc | Knockdown |
| | for comparison | GTCGACGAT | | |
| 450 | shRNA guide for | AAAGTTCTGTTTGCCCTGAT | Gluc | Knockdown |
| | comparison | CCTCGAGGATCAGGGCAAA | | |
| | | CAGAACTTT | | |

Table 9 shows the representative Cas7-11 orthologs of FIG. 1F.

TABLE 9

| SEQ ID NO/ | |
|---|---|
| NAME | SEQUENCE |
| SEQ ID NO: 451 | RDMATSAHTTIDKETKRTKENHLINLEVVRIEDN-RYLKGSVEMIN- |
| MBD3181016.1 | |
| SEQ ID NO: 452 | DQVRTSAHNKINRATGRVEKDLLFSYELG--ANT-VPLEGTIERVNP |
| MBC8521845.1 | |
| SEQ ID NO: 453 | DMVRTSTHNKISRATGRVEKDHLFSYELG--ANT-TPFEGMIEPVNH |
| RCV63412.1 | |
| SEQ ID NO: 454 | GTTRVSLHNRVDPTTGRVPEETFFSFEVG--SAE-K-FTGRIERSGP |
| MBC8461765.1 | |
| SEQ ID NO: 455 | EPIRISTHNRIDPATGRVPKDLLFTFEMG--SPA-V-FRGYIERIGI |
| HHR29383.1 | |
| SEQ ID NO: 456 | LQTFLYTQARLDRPTRTAVPGALYSSEFG--LRE-ITFKGSVTGWLE |
| WP_129889723.1 | |
| SEQ ID NO: 457 | NQKDVYTQVRLDRPTRTAVEGALYTSEFG--TRG-LTFHGDITGSLT |
| WP_174676620.1 | |
| SEQ ID NO: 458 | LFLNTRTGTKIDRRTGTVQEDHLFTTESG--IKG-ITLTSKIYGRGN |
| TFH44231.1 | |
| SEQ ID NO: 459 | YGQSIRSNNRIDPKTRTAANDHLFFTECV--DPD-TYFIGEIRSSSN |
| MAT45258.1 | |
| SEQ ID NO: 460 | YGRLLRTGIGIDRKLRVVVPDRLFTNECV--GTK-IPFTGRIIGEFP |
| WP_075063448.1 | |
| SEQ ID NO: 461 | EIAVSRVVNRVDPNSGKA-KDFFRVWEID--HKLCPNFLGKMSISLS |
| HreCas7-11 | |
| SEQ ID NO: 462 | DIVQKRILNRVDQTCGKA-KDFFKVCEVD--HIACPTLNGIIRINDE |
| CmaCas7-11 | |
| SEQ ID NO: 463 | ETAIERIVNRVDPSSGKA-KDYMRIWEID--PLVCSQFNGIITIN-- |
| HvmCas7-11 | |
| SEQ ID NO: 464 | EIGSRRTINRVDDETGKA-HDFFSIWEVD--A--VREFQGEIVLAAD |
| SmCas7-11 | |
| SEQ ID NO: 465 | EIGRLRTLNRIDRLTTKA-QDFFRIYEVD--Q--VRDFFGTITLAGD |
| OmCas7-11 | |
| SEQ ID NO: 466 | AIGSQRVLNRVDFKSGKA-HDFFKAYEVD--HTRFPRFEGEITIDNK |
| DisCas7-11 | |
| SEQ ID NO: 467 | EIAVRRVVNRVDYASGKA-HDFFRIFEVD--HIAFPCFHGEIAFGEN |
| DsbaCas7-11 | |
| SEQ ID NO: 468 | DIGTQRTLNRVDYATGKA-HDFFKVWEID--HSLLSVFQGKISIADN |
| SstCas7-11 | |
| SEQ ID NO: 469 | DIASGRILNRVDFDTGKA-KDYFRTWEAD--YETYGTYTGRITLRNE |
| CsbCas7-11 | |
| SEQ ID NO: 470 | DIASERILNRVDYTTGKA-HDYFKVWEVD--DDQWWQFTGTITMHDD |
| CjcCas7-11 | |
| SEQ ID NO: 471 | DVCKERVLNRVDFRTGKA-QDYFRVFEID--HEDWGVYTGEITITEP |
| CbfCas7-11 | |
| SEQ ID NO: 472 | ELAPKRALNRIEQYTGKA-QDYFTVYEAL--NKEFWTFKGRIRIKED |
| HvsCas7-11 | |
| SEQ ID NO: 473 | EIGIRRILNRVDFTTGKA-QDYFYVWEVE--HSMCPKFQGTVKINED |
| HsmCas7-11 | |
| SEQ ID NO: 474 | DIAQKRTSNRVDFFSKKA-HDHYGVWEVT--A--VKNLLGYIYISDA |
| FmCas7-11 | |
| SEQ ID NO: 475 | QLIRPRILNRMDPGTSKA-RDYFRVFEIE--NQLCSQFRGWIWLSGD |
| DpbaCas7-11 | |

Table 10 shows the representative Cas7-11 orthologs of FIG. 1G.

TABLE 10

| SEQ ID NO/ | |
|---|---|
| NAME | SEQUENCE |
| SEQ ID NO: 476 | QRI----------------TPDAPDADP-YWQADIALDADGRPYLPG |
| WmCas7x3 | |
| SEQ ID NO: 477 | EEMDKQSG-----------ESVDKKQNN-SWIQAIALDLNKKPYIPG |
| NisCas7x3 | |
| SEQ ID NO: 478 | E------------------KERDENKET-RWLEAVALDHKGQPYIPG |
| GwCas7x3 | |
| SEQ ID NO: 479 | VTRP---------------ELTVADRDELVDINAVVTDYTGKPYLPG |
| HvmCas7x3 | |
| SEQ ID NO: 480 | TDRPGLEI-----------EQKDGSKVK-AEINAFIKDSNGKPYLPG |
| DsbCas7x3 | |
| SEQ ID NO: 481 | IPVT---I-----------KDTQGKNRE-VEVNSVITGKAALPIIPG |
| Gam:Cas7x3 | |
| SEQ ID NO: 482 | VEHD---------------LIKNDDGTP-VQINALITGAGGLPIIPG |
| DesCas7x3 | |
| SEQ ID NO: 483 | AEIE---I-----------NDSQGDRRQ-VQANAIIRGKDDKPIIPG |
| GabCas7x3 | |
| SEQ ID NO: 484 | CEHD---------------DVKNNDGEP-VKINACIKGSKGRPIIPG |
| MetCas7x3 | |
| SEQ ID NO: 485 | CYRP---------------ELTNADQKP-VDINACIKGANNLPIIPG |
| MebCas7x3 | |
| SEQ ID NO: 486 | -----------------EGQKQTDDQAE-SLHLRTLRDGHGRFRIPF |
| GwCas7-11 | |
| SEQ ID NO: 487 | ----------------------SEGQTS----QAIILCPDGSYRLPR |
| HreCas7-11 | |
| SEQ ID NO: 488 | ----------------------KTSHTR----STIFLNMNGQFCIPR |
| CmaCas7-11 | |
| SEQ ID NO: 489 | ----------------------QEGQTS----ATILLNRDGYFRLPR |
| HvmCas7-11 | |
| SEQ ID NO: 490 | -------------------PIDDDQTS----SRTL VDRDGRYRLPR |
| SmCas7-11 | |
| SEQ ID NO: 491 | ---------------------GSHDHTS----RKILLTRDFYYRLPR |
| OmCas7-11 | |
| SEQ ID NO: 492 | ---------------------EDAKQTD----LQVLLTPDNKYRLPR |
| DisCas7-11 | |
| SEQ ID NO: 493 | ---------------------KEKKQTD----LMLLLDGQNHYRIPR |
| DsbaCas7-11 | |
| SEQ ID NO: 494 | ---------------------EDAKHTN----LKVLLDRQNRYRLPR |
| SstCas7-11 | |
| SEQ ID NO: 495 | ---------------------GAGGHTD----LSILLGKDGHYRVPR |
| SybCas7-11 | |
| SEQ ID NO: 496 | SDSIPGKEK------KSEDSLVINEHTS----FNILLDKENRYRIPR |
| CsbCas7-11 | |
| SEQ ID NO: 497 | FDSTQDDLDLVPDIVNTDEKLEANEQTS----FRILMDKKGRYRIPR |
| CjcCas7-11 | |
| SEQ ID NO: 498 | ------------------EQTEEVEHTS----LRLVMDKKGRFRIPR |
| CbfCas7-11 | |
| SEQ ID NO: 499 | ----------------------DEEKAE----GAILLTPDNRFRLPR |
| HvsCas7-11 | |
| SEQ ID NO: 500 | ---------------------KTEEQID----MQILLTKDGRYRLPR |
| HsmCas7-11 | |
| SEQ ID NO: 501 | ---------------------QAGKQTS----MPTLQDSNDHFRLPR |
| FmCas7-11 | |
| SEQ ID NO: 502 | ---------------------ATETHTD----LPILLTSDRHFRIPR |
| DpbaCas7-11 | |

Table 11 shows the representative Cas7-11 orthologs of FIG. 1H.

TABLE 11

| SEQ ID NO/ | |
|---|---|
| NAME | SEQUENCE |
| SEQ ID NO: 503 | HRKPQDNAPDGTPRQRGDRA---------------LLPGASLRGRLRS |
| WxxCas7x3 | |
| SEQ ID NO: 504 | KKDEAKNEADAKPRTNHQGQV--------------ILPASSLRGRLRA |
| NisCas7x3 | |
| SEQ ID NO: 505 | KADDADAV----PRRTHDDKI--------------VLPASSLRGRLRT |
| GwCas7x3 | |
| SEQ ID NO: 506 | KKKRRSNTPNLRPLRDAIGRP--------------CLPESSVRGALRA |
| HvmCas7x3 | |
| SEQ ID NO: 507 | KKRKED--IDHQPLRDSAGNA--------------RLPAKSIRGAMRS |
| DsbCas7x3 | |
| SEQ ID NO: 508 | DKCKAEDTPDIYPLEEKNGVP--------------AFPVRSFRGAIRS |
| CamCas7x3 | |
| SEQ ID NO: 509 | NTPDITQAPDMVPLVDEDGNP--------------MLPASSFRGALRA |
| DesCas7x3 | |
| SEQ ID NO: 510 | SKGDGDQPAVHQPLTDRSNNP--------------ILPARSFRGAIRA |
| GabCas7x3 | |
| SEQ ID NO: 511 | LSSNENAKTDHYPLLDKNRNP--------------YLPVSSFRGVLRS |
| MetCas7x3 | |
| SEQ ID NO: 512 | LEADPKTKIDHYPLLDNHKKP--------------RLPSASIRGVLRS |
| MebCas7x3 | |
| SEQ ID NO: 513 | ESDAPDNVAYKKPVVQYDETGRLRTTDPGPVEMLTCLKGEGVRGVVAY |
| GwCas7-11 | |
| SEQ ID NO: 514 | DHDNKDAVMVQKTVLFVDESG------NYSQMPHHFLKGSGIRGACRF |
| HreCas7-11 | |
| SEQ ID NO: 515 | DNRNPDAVMVKKTILVYEQDSSTHKNVPKEVPKY-FIKSETIRGLLRS |
| CmaCas7-11 | |
| SEQ ID NO: 516 | DPRNTDAIMVRKTVFCPDPNA---KNRPAPATVY-MIKGESIRGILRS |
| HvmCas7-11 | |
| SEQ ID NO: 517 | NSDTADIISFRRTVVDNGEVL------REP-----VLRGEGLRGLLRT |
| SmCas7-11 | |
| SEQ ID NO: 518 | -RHEEDSVYFQKRIFTSDGRV-----VLVP-----ALRGEGLRGLLRT |
| OmCas7-11 | |
| SEQ ID NO: 519 | DKRGTDVVTFVKYKAEGEEAK------PVC-----AYKAESFRGVIRS |
| DisCas7-11 | |
| SEQ ID NO: 520 | TEDVADIVSFKKYTQGGEKIIY-------------AYKSESFRGVVRT |
| DsbaCas7-11 | |
| SEQ ID NO: 521 | EKDGSDIVSFRKYADDSGKEVY-------------AYKAESFRGVVRA |
| SstCas7-11 | |
| SEQ ID NO: 522 | ERIGFDNIAYEKRRYNGETNT------TESIP---AVKGETFRGIVRT |
| SybCas7-11 | |
| SEQ ID NO: 523 | EPGNRDAIAYKKRVYNDGNNA------IEPEPRF-AVKSETHRGIFRT |
| CsbCas7-11 | |
| SEQ ID NO: 524 | NKDNIDCIAYEKRKWENGGIK------FVP-----TIKGETIRGIVRM |
| CjcCas7-11 | |
| SEQ ID NO: 525 | DPNNVDAIVFEKMKLDGDQVK------YLP-----AIKGETIRGIVRT |
| CbfCas7-11 | |
| SEQ ID NO: 526 | EGKAPDAVFFKKYVFENGKIE------EKP-----CFKAESIRGIFRT |
| HvsCas7-11 | |
| SEQ ID NO: 527 | DSSNTDLVTFKKYKLEESKEVF-------------AIKGESIRGVFRT |
| HsmCas7-11 | |
| SEQ ID NO: 528 | -HGNTDSVFYKKPILKSGEKE------PSYQW---AIKSDTVRGLIRS |
| FmCas7-11 | |
| SEQ ID NO: 529 | -RNESDAVFYQKSVAGEKGPVY-------------ALKGEGLRGIVSS |
| DpbaCas7-11 | |

Table 12 shows the representative Cas7-11 orthologs of FIG. 1I.

TABLE 12

| SEQ ID NO/ | |
|---|---|
| NAME | SEQUENCE |
| SEQ ID NO: 530 | PATLVDHDMLAIDRFTGGGKDGAKFK----LRYAECP-TLEGQLSLDL |
| WmCas7x3 | |
| SEQ ID NO: 531 | LATLTRHEMVAIDRFTGGGKEGAKFN----VDYIECP-TLTGAIYLDL |
| NisCas7x3 | |
| SEQ ID NO: 532 | SIKTRRHEMLAIDRFTGGGKDGAKFN----VDYVECP-TLAGKLSLDL |
| GwCas7x3 | |
| SEQ ID NO: 533 | EDCDNIQEFVAIDRFTGGAKDKAKFN----AEYIGSP-RFTGTIALDK |
| HvmCas7x3 | |
| SEQ ID NO: 534 | TRQETVQDFVAIDRFHGGGKETAKFD----ASFSWRP-QYSILMHIPS |
| DsbCas7x3 | |
| SEQ ID NO: 535 | LKPFIIQEFVAIDRFHGGGKDEAKFN----AAHYQAP-VFKGKVRVSQ |
| GamCas7x3 | |
| SEQ ID NO: 536 | FRREQEQTFVAIDRFHGGCKEGALYT----IRHAESP-RFEGHLVIDP |
| DesCas7x3 | |
| SEQ ID NO: 537 | YRPAKTQQFVAIDRFHGGGKDGALFS----IKYFERP-VLKGGISLKL |
| GabCas7x3 | |
| SEQ ID NO: 538 | KSKKTKQDFVAIDRFHGGGKDGAKFD----ATHFERP-EFEGAISFSP |
| MetCas7x3 | |
| SEQ ID NO: 539 | ANELKTQEFIAIDRFHGGGKDGAKFN----AKHSERP-YFQGRITLSP |
| MebCas7x3 | |
| SEQ ID NO: 540 | TPHAMRSDRVALDVF-GGAMPEAKFDDRPLAASPGKPLNFKSTIWYRE |
| GwCas7-11 | |
| SEQ ID NO: 541 | EVEAIKCDHVAIDRFHGGTVHRMKYDDYPLPGSPNRPLRIKGNIWVKR |
| HreCas7-11 | |
| SEQ ID NO: 542 | NVSDCCIDHVAIDRFTGGGVEKMKFNDYPLSASPKNCLNLKGSIWITS |
| CmaCas7-11 | |
| SEQ ID NO: 543 | SVSDKKMDHVAIDRFTGGGVDQMKFDDYPLPGCPAQPLILEGKFWVKD |
| HvmCas7-11 | |
| SEQ ID NO: 544 | TVADKRLDHVAIDRFDQSVVE--KYDDRPLVGSPKQPLVFKGCFWVQT |
| SmCas7-11 | |
| SEQ ID NO: 545 | TWNDKKIDHVSCSRFDASVVN--KFDDRSLVGSPDSPLHFEGTFWLHR |
| OmCas7-11 | |
| SEQ ID NO: 546 | DPEPVTFDHVAIDRFTGGAADKKKFDDSPLPGSPARPLMLKGSFWIRR |
| DisCas7-11 | |
| SEQ ID NO: 547 | EPEPRRFDHVAIDRFTGGAVNQKKFDDRSLVPGKEGFMTLIGCFWMRK |
| DsbaCas7-11 | |
| SEQ ID NO: 548 | QPEPMIFDHVAIDRFTGGAVDKKKFDDCSLPGTPGHPLTLKGCFWIRK |
| SstCas7-11 | |
| SEQ ID NO: 549 | AWTRKHLDHVAIDRFHGGAEENMKFDTYALAASPTNPLRMKGLIWVRS |
| SybCas7-11 | |
| SEQ ID NO: 550 | EKLEKHIDHVAIDRFTGGALDKAKFDTYPLAGSPKKPLKLKGRFWIKK |
| CsbCas7-11 | |
| SEQ ID NO: 551 | SYKKKLIDHVAIDRFHGGAEDKMKFNTLPLAGSFEKPIILKGRFWIKK |
| CjcCas7-11 | |
| SEQ ID NO: 552 | KDIAKKIDHVAIDRFTGGARDQMKFDTLPLIGSPERPLRLKGLFWMRR |
| CbfCas7-11 | |
| SEQ ID NO: 553 | GQEEKFFDHVAIDRFLGGAKEKYKFDDKPIIGAPDTPIVLEGKIWVKK |
| HvsCas7-11 | |
| SEQ ID NO: 554 | DSAPKRLDHVAIDRFTGGAKEQAKFDDSPLIGSPDSPLEFTGIVWVRD |
| HsmCas7-11 | |
| SEQ ID NO: 555 | EIKTYRMDHVAIDRISGGAVDQCKYDDEPLVGTSKHPLVFKGMFWINR |
| FmCas7-11 | |
| SEQ ID NO: 556 | EVREKLFDHVSIDRFTGGAANKLKFDDKPLVG---NPLVFQGVFWVHQ |
| DpbaCas7-11 | |

Table 13 shows the representative Cas7-11 orthologs of FIG. 1J.

TABLE 13

| SEQ ID NO\ | |
|---|---|
| NAME | SEQUENCE |
| SEQ ID NO: 557 | PVAGFALQNQPAIPATSLRGLLSSLFESISGSNLRVLHPTPYSIRKTT |
| WmCas7x3 | |
| SEQ ID NO: 558 | EARPFKLNGKHAIPATSLRGMLSSLFESVSNSNFRVLHPEHYSVRKSL |
| NisCas7x3 | |
| SEQ ID NO: 559 | SLHPFKLNNGLAIPATSLRGMISSLFESVSNSNFRVLDEKTYSMRKTM |
| GwCas7x3 | |
| SEQ ID NO: 560 | EILPFTLGGKPAIPATSLRGMLSSIAEAASNSSLRVLEDKTLSYRKSM |
| HvmCas7x3 | |
| SEQ ID NO: 561 | QVDHYTENGEIAIPATTLRGLLSSLSEAASNSSMRVLDDGMMSYRQPV |
| DsbCas7x3 | |
| SEQ ID NO: 562 | LRHPYQLDGEPAIPSTSLRGLISTMTEAAANCAMRVLDSEIISYRKPM |
| GamCas7x3 | |
| SEQ ID NO: 563 | RIENYRLGNRIAIPAASLRGMLSSLAEAASNSAMRVLHQGILSYRKKA |
| DesCas7x3 | |
| SEQ ID NO: 564 | LKEHYQLNNKLAIPATSLRGLISSLAEAASNSALRVLDNGVLSYRKPA |
| GabCas7x3 | |
| SEQ ID NO: 565 | EINNYRLNGELAIPATSLRGMISSLAEAASNSAMRVLDNGLLSYRKTA |
| MetCas7x3 | |
| SEQ ID NO: 566 | ELDNYRLNGQLAIPATSLRGMISSLAEAASNSAMRVLDNGLLSYRKDA |
| MebCas7x3 | |
| SEQ ID NO: 567 | TQRFHQINDEIGLPGASLRGMVLSNYQILTNSCYRNLKATEEITRRMP |
| GwCas7-11 | |
| SEQ ID NO: 568 | NYRFFRINDELAIPGSELRGMVSSVYEALTNSCFRIMEEGRYLSRRMG |
| HreCas7-11 | |
| SEQ ID NO: 569 | NYAFFRINDHIAIPGASIRGMISSVFETLTHSCFRVMDDKKYLTRRVI |
| CmaCas7-11 | |
| SEQ ID NO: 570 | NYGFFRINGNVAIPGSSIRGMISSVFEALTNSCFRVFDQERYLSRSEK |
| HvmCas7-11 | |
| SEQ ID NO: 571 | NFPFFKINDEIMLPGAPLWAAVSQVYEALTNSCFRVMKQKRFLSWRME |
| SmCas7-11 | |
| SEQ ID NO: 572 | EYPSFRLNNTPMIPGAGLRAAVSQVYEVLTNSCIRIMDQGQTLSWRMS |
| OmCas7-11 | |
| SEQ ID NO: 573 | SYAFFRLHKQIMIPGSELRGMVSSVYETVTNSCFRIFDETKRLSWRMD |
| DisCas7-11 | |
| SEQ ID NO: 574 | SYRFFTLNRVPMIPGSEIRGMISSVFEALSNSCFRIFDEKYRLSWRMD |
| DsbaCas7-11 | |
| SEQ ID NO: 575 | SYAFFSVNGDIMLPGSEIRGMLSSVYEALTNSCFRVFDEGYRLSWRME |
| SstCas7-11 | |
| SEQ ID NO: 576 | MCKFFSVAGKPMIPGSEIRGMISSVYEALTNSCFRVFDEEKYLTRRVQ |
| SybCas7-11 | |
| SEQ ID NO: 577 | NYKFFNINGELMIPGSELRGMLRTHFEALTKSCFAIFGEDSTLSWRMN |
| CsbCas7-11 | |
| SEQ ID NO: 578 | NYQFFHINDEIMVPGSEIRGMISSVYEAITNSCFRVYDETKYITRRLS |
| CjcCas7-11 | |
| SEQ ID NO: 579 | TYDFFQMNNAIMIPGSEIRGMISAVYEAMTNSCFRIFHEKQYLTRRIS |
| CbfCas7-11 | |
| SEQ ID NO: 580 | YYHFLKIDNKPIIPGAEIRGAVSSIYEALTNSCFRVFGEKKVLSWRME |
| HvsCas7-11 | |
| SEQ ID NO: 581 | VYKFFRLGGHLCIPAAEIRGMISSVYEALTNSCFRVFDEKRLISWRMT |
| HsmCas7-11 | |
| SEQ ID NO: 582 | SFRFFRIDDEVLIPGSEIRGMVSTVFEALTGSCFRVINQKAHLSWRIN |
| FmCas7-11 | |
| SEQ ID NO: 583 | RFKFMRMGSQAAIPGSAIRSMTSSVFEALTNSCFRVLDQKSHLSWRME |
| DpbaCas7-11 | |

Table 14 shows the representative Cas7-11 orthologs of FIG. 1K.

TABLE 14

| SEQ ID NO\ | |
|---|---|
| NAME | SEQUENCE |
| SEQ ID NO: 584 | E--------------------ALSA--IGRI-VE----RNGELKLYPL |
| WmCas7x3 | |
| SEQ ID NO: 585 | DYV------------------ALSA--MGRI-VD----DQGELKLQPL |
| NisCas7x3 | |
| SEQ ID NO: 586 | Q--------------------SLSA--MGRI-VR----HDQKLYLLPL |
| GwCas7x3 | |
| SEQ ID NO: 587 | ANRN--------------EDKPLSA--LGMI-KKIETGDKVEYRLLPL |
| HvmCas7x3 | |
| SEQ ID NO: 588 | SG-------------------SLSA--IGMV-VI----RDGKKFIYPL |
| DsbCas7x3 | |
| SEQ ID NO: 589 | PSH------------------ILSA--LGMV-TK----RGEDFWLIPL |
| GamCas7x3 | |
| SEQ ID NO: 590 | N--------------------ALRE--IGMI-VL----RDGKRFILPL |
| DesCas7x3 | |
| SEQ ID NO: 591 | R--------------------ALRK--IGIL-FK----REEQWRLVQM |
| GabCas7x3 | |
| SEQ ID NO: 592 | D--------------------ALRK--VGMV-IY----VDNKSFIIKL |
| MetCas7x3 | |
| SEQ ID NO: 593 | L--------------------ALSK--IGITFIN----RQGQWQLIPM |
| MebCas7x3 | |
| SEQ ID NO: 594 | DEA------------------KYRK--AGRV-TVSGDGAQKKYSIQEM |
| GwCas7-11 | |
| SEQ ID NO: 595 | D--------------------EFKDFHPGIV--------VDGAKIREM |
| HreCas7-11 | |
| SEQ ID NO: 596 | ESETTQ-KRKSGRYQVEESDPDLF---PGRV-QK----KGNKYKIEKM |
| CmaCas7-11 | |
| SEQ ID NO: 597 | DPT------------------ELTKYYPGKV-KR----DGNKFFILKM |
| HvmCas7-11 | |
| SEQ ID NO: 598 | E--------------------DYKDFYPGRV-------LDGGKQIKKM |
| SmCas7-11 | |
| SEQ ID NO: 599 | SEHK-----------------DYQ---PGKI-------TDNGRKIQPM |
| OmCas7-11 | |
| SEQ ID NO: 600 | DHQN-----------------VLQDFLPGRV-------TADGKHIQKF |
| DisCas7-11 | |
| SEQ ID NO: 601 | DVK------------------ELEQFKPGRV-------ADDGKRIEEM |
| DsbaCas7-11 | |
| SEQ ID NO: 602 | DRN------------------VLMQFKPGRV-------TDNGLRIEEM |
| SstCas7-11 | |
| SEQ ID NO: 603 | KKGA-----------------KSSELVPGII-VWG---QNGGLAVQQV |
| SybCas7-11 | |
| SEQ ID NO: 604 | DEKDYKIDSNSIRKMESQRNPKYRI--PDEL-QKELRNSGNGLFNRLY |
| CsbCas7-11 | |
| SEQ ID NO: 605 | EKKD---ESNDKNKSQDDASQKIR---KGLV-KK----TDEGFSIIEV |
| CjcCas7-11 | |
| SEQ ID NO: 606 | EDK------------------ELREFIPGIV-RI----INGDVYIEKA |
| CbfCas7-11 | |
| SEQ ID NO: 607 | KDAK-----------------EFM---PGRV-SK----KKGKLYMVKM |
| HvsCas7-11 | |
| SEQ ID NO: 608 | EEAK----RPDPKKSEEQNRMRFR---PGRI-IK----KDKKFYAQEM |
| HsmCas7-11 | |
| SEQ ID NO: 609 | DMAK-----------------HYR---PGRI-IQ----NNEKMFIQPY |
| FmCas7-11 | |
| SEQ ID NO: 610 | DDAG-----------------DYK---PGRF-EK----KDDKAVIRKF |
| DpbaCas7-11 | |

Table 15 shows sequence alignments of cas nucleases.

DOMAINS
Mutated residues; catalytic residues
Domain 1 - Csm3-like (group 7 RAMP) with abnormal “G-rich” loop
Domain 2 - putative small subunit (no sequence similarity but there is a conserved “W” and
four large alpha
helices predicted by Jpred in this region)
Domain 3 - Csm3-like (group 7 RAMP), could be catalytic
Domain 4 - Csm3-like (group 7 RAMP), * - catalytic aspartates based on Zhu X, Ye K.
Nucleic Acids Res. 2015 Jan;43(2):1257-67; https://doi.org/10.1093/nar/gku1355)
Domain 5 - Csm3-like with large insertion or subdomain (group 7 RAMP)
WmCas7x3, GwCas7x3, SER16298.1, PID64649.1, WP_031436019.1, HEB50754.1 - III-D systems
SECTION 1
SEQ ID NO: 611
WmCas7x3 ----------------------------------------------------------------------------------
-------------------------------------------------------------------------------------------
--------------------------
SEQ ID NO: 612
SER16298.1 --------------------------------------------------------------------------------
-------------------------------------------------------------------------------------------
----------------------------
SEQ ID NO: 613
GwCas7x3 ----------------------------------------------------------------------------------
-------------------------------------------------------------------------------------------
--------------------------
SEQ ID NO: 614
PID64649.1 --------------------------------------------------------------------------------
-------------------------------------------------------------------------------------------
----------------------------
SEQ ID NO: 615
WP_031436019.1 ----------------------------------------------------------------------------
-------------------------------------------------------------------------------------------
--------------------------------
SEQ ID NO: 616
HEB50754.1 --------------------------------------------------------------------------------
-------------------------------------------------------------------------------------------
----------------------------
SEQ ID NO: 617
HreCas7-11 --MSVEEFYVRLTFL-EPFRVVPWVRNGDERKGDRIYQRGGTYARWHKI-----
NDSHGQPYITGTMLRSAVLREIENTLTLHNTY----GCCPGGT--RTTEGKLEKPL-YLRRRD--
GFEFENH-AEKPC-----SEEDPCPLCLIQGRFDKLRRDEKKQFVRQGNI-SFCSVNFSNLNISS---
GIKSFSWE---EI
SEQ ID NO: 618
CmaCas7-11 ----MLKLKVKITYF-QPFRVIPWIKEDDRNSDRN-YLRGGTFARWHKDKK---
DDIHGKPYITGTLLRSALFTEIEKIKIHHSDFIH---CCNAID----RTEGKHQPS-FLRKRP----
VYTENKNIQAC--------NKCPLCLIMGRGDDRGEDLKKKKHYNGKHYQNWTVHFSNFD------
TQATFYWK---DI
SEQ ID NO: 619
HvmCas7-11 ----MTQITIQVTFF-HPFRVVPWNHRDHRKTDRK-YLRGGTFAKWHCTAS---
EGKSGRPYITGTLLRSALFAEIEKLIAFHDPF----KCCRGKD---KTENGNAKPL-FLRRRP-----------
RADC-----DPCGTCPLCLLMGRSDTVRRDAKKQKK-------DWSVHFCNLR-EA---TERSFNWK---ET
SEQ ID NO: 620
SmCas7x3 -----MRLKINIHFL-EPFRLIEWHEQDRRNKGNSRWQRGQSFARWHRRKD---NDQ-
GRPYITGTLLRSVVIRAVEEELARPDTAWQ---SCGGLF---ITPDGQTKPQ-HLRHRA--TVRARQT-
AKDKC---A-DRQSACPFCLLLGRFDQVGKDGDKKGEGL-----RFDVRFSNLD-LPKDFSPRDFDGPQ--
EI
SEQ ID NO: 621
oral_meta MIPDLRSLVVHISFL-TPYRQAPWFPPEKRRNNNRDWLRMQSYARWHKVAP---EE--
GHPFITGTLLRSRVIRAVEEELCLANGIWRGVACCPGEF----NSQAKKKPK-HLRRRT--TLQWYPE-
GAKSCSKQD-GRENACPFCLLLDRFGGEKSEEGRKKNN------DYDVHFSNLNPFYPGSSPKVWSGPE-
-EI
SEQ ID NO: 622
DisCas7-11 ---MTTTMKISIEFL-EPFRMTKWQESTRRNKNNKEFVRGQAFARWHRNKK---
DNTKGRPYITGTLLRSAVIRSAENLLTLSDGKISEKTCCPGKF----DTEDKDRLL-QLRQRS--TLRWT---
DKNPC--PD-NAETYCPFCELLGRSGNDGKKAEKKDW-------RFRIHFGNLS-LP---GKPDFDGPK--AI
SEQ ID NO: 623
DsbaCas7-11 -------MKITLRFL-EPFRMLDWIRPEERISGNKAFQRGLTFARWHKSKA---DDK-
GKPFITGTLLRSAVIRAAEHLLVLSKGKVGEKACCPGKFLTETDTETNKAPTMFLRKRP--TLKWT---
DRKGC-----DPDFPCPLCELLGPGAVGKKEGEAGI--------NSYVNFGNLS-FP---GDTGYSNAR--EI
SEQ ID NO: 624
SstCas7-11 -----MIINITVKFL-GPFRMLEWTDPDNRNRKNREFMRGQAFARWHNSNP---QKG-
SQPYITGTLVRSAVIRSAENLLMLSEGKVGKEKCCPGEF--RTENRKKRDAMLHLRQRS--TLQWKT--
DKPLC-----NGKSLCPICELLGRRIGKTDEVKKKG--------DFRIHFGNLT------PLNRYDDPS--DI
SEQ ID NO: 625
CsbCas7-11 -----MNITVELTFF-EPYRLVEWFDWDARKKSHS-AMRGQAFAQWTWKGK---
GRTAGKSFITGTLVRSAVIKAVEELLSLNNGKWEGVPCCNGSF--QTDESKGKKPS-FLRKRH--
TLQWQAN-NKNIC-----DKEEACPFCILLGRFDNAGKVHERNK--------DYDIHFSNFD-LD---
HKQEKNDLRLVDI
SEQ ID NO: 626
CjcCas7-11 ---MHTILPIHLTFL-EPYRLAEWHAKADRKKNKR-YLRGMSFAQWHKDKD---GI--
GKPYITGTLLRSAVLNAAEELISLNQGMWAKEPCCNGKF-----ETEKDKPA-VLRKRP--TIQWKTG-
RPAICDPEKQEKKDACPLCMLLGRFDKAGKRHRDNKYDKH----DYDIHFDNLN-LI---TDKKFSHPD--
DI
SEQ ID NO: 627
CbfCas7-11 MSKTDDKIDIKLTFL-EPYRMVNWLENGLRMTDPR-YLRGLSFARWHRNKN---
GKA-GRPYITGTLLRSAVIRAAEELLSLNLGKWGKQLCCPGQF--ETEREMRKNKT-FLRRRP--
TPAWSAETKKEIC---T-THGSACAFCLLLGRRLHGGKEDVNEDAPGS---CRKPVGFGNLS-LP---
FQPTKRQIQ--DV
SEQ ID NO: 628
HvsCas7-11 ----MKSIPITLTFL-EPYRILPWAEKGKRDKKE--YLRGANYVRLHKDKN---GK--
FKPYITGTLIRSAVLSAIEMLLDITNGEWNGKECCLAKF-----HTEGEKPS-FLRKKP----IYIRAEKDEIC-
--T-SRETACPLCLILGRFDKAEKKEKDKE--------KFDVHFSNLN-LY---SSKEFSTIE--EL
SEQ ID NO: 629
HsmCas7-11 ----MTKIPISLTFL-EPFRLVDWVSESERDKSE--
FLRGLSFARWHRIKNQREDENQGRPYITGTLLRSAVIKAAEELIFLNGGKWQSEECCNGQF--
KGSKAKYRKVE-CPRRRHRATLKW----TDNTC---S-DYHNACPFCLLLGCLKPNSKE-------------
NSDIHFSNLS-LP---NKQIFKNPP--EI
SEQ ID NO: 630
FmCas7-11 ----MPRFQLSLTFFDEPFRLIEWTDKSNRNSANTQWMRGQGFARWHKITL---EK--
GFPFVTGTAVRSKIIREVEALLSRNKGTWNGIPCCSGFF-----DTKGPSPT-HLRYRP--TLEWEY--
GKTVC---T-SEADVCPLCLLLGRFDQAGKKSDTPCQST-----DYHVHWENLS--A---GVAQYR-LE--DI
SEQ ID NO: 631
DpbaCas7-11 --------------F-E------------------------SYARWCKSNS---GL--
WKPYIPGTLLRSAVLESVEYLLAL-IGSKNKVEICPGLY---TQSENNPDTK-YLRRRP--WYELHA--
QKEIC---K-TRDTACPLCLLMRTKLDNDGDGETEK----------NVKFGNL-------YPTSPLEPL--QK
Domains
.........................................................................
.........................................................................
........................................................
SECTION 2 (SEQ ID NOS 611-631, continued from section above)
D177 atypical G-rich loop
WmCas7x3 ---------------------------------------------------------------------------------
------------------------------------------------------------------------------------------
----------------------------
SER16298.1 -------------------------------------------------------------------------------
------------------------------------------------------------------------------------------
------------------------------
GwCas7x3 ---------------------------------------------------------------------------------
------------------------------------------------------------------------------------------
----------------------------
PID64649.1 -------------------------------------------------------------------------------
------------------------------------------------------------------------------------------
------------------------------
WP_031436019.1 ---------------------------------------------------------------------------
------------------------------------------------------------------------------------------
----------------------------------
HEB50754.1 -------------------------------------------------------------------------------
------------------------------------------------------------------------------------------
------------------------------
HreCas7-11 AVSRVVNRVDPNSGKAKDFFRVWEIDHKLCPNFLGKMSISLS----
EKLEDVKALLAVGLAQVNVLSGALCRVDIID-------------------------
PETQKDTVHQHLIQQFVTRIQDKEKGDAADIPAFTLPPAGLSPSSNEWNDTIKSLAEKIR--
KIKELEQGQKLRQMADVIREL-RRKTPAYLDQ----
CmaCas7-11 VQKRILNRVDQTCGKAKDFFKVCEVDHIACPTLNGIIRINDEKLSQEEISKIKQLIAVGLAQIESLAGGI
CRIDITN--------------------------------------QNHDDLIKSFFETKPSKILQPNL---
KESGEERFELAKLELLAEYLT-QSFDANQKEQQLRRLADAIRDL-RKYSPDYLKD----
HvmCas7-11 AIERIVNRVDPSSGKAKDYMRIWEIDPL VCSQFNGIITINLD---
TDNAGKVKLLMAAGLAQINILAGSICRADIIS------------------
EDHDALIKQFMAIDVREPEVSTSFPLQDDELNNAPAGCGDDEISTDQPVGHNLVDRVRISKIAESIE-
DVFSQEQKAQQLRRMADAIRDL-RRSKPDETTLDA--
SmCas7x3 GSRRTINRVDDETGKAHDFFSIWEVDA--VREFQGEIVLAAD-L--
PSRDQVESLLHHALGFVDRLCGARCVISIAD--------------------------------------------------
QKPAEREERTV----AAGDEKATIADYDQVKGLPY----------TRLRPLADAVRNL-RQLDLAELNKPDGK
oral_meta GRLRTLNRIDRLTTKAQDFFRIYEVDQ--VRDFFGTITLAGD-L--
PRKVDVEFLLRRGLGFVSTLCGAQCEIKVVD--------------------------------------------------
LKKKQNNKEDSI----LPVSEVPFFLEPEVLAKMCQ-DVFPS----GKLRMLADVILRL-REEGPDNLT-----
DisCas7-11 GSQRVLNRVDFKSGKAHDFFKAYEVDHTRFPRFEGEITIDNK-V--S--
AEARKLLCDSLKFTDRLCGALCVIRFDE-------------------------------------------------YTPAADSGKQT-
---ENVQAEPNANLAEKTAEQII-SILDDNKKTEYTRLLADAIRSL-RRSSKLVAG-----
DsbaCas7-11 AVRRVVNRVDY ASGKAHDFFRIFEVDHIAFPCFHGEIAFGEN-V--S--
SQARNLLQDSLRFTDRLCGALCVIRYDG--------------------------------------------DIPKCGKTAPLPETESI
----QNAAEETARAIVRVFHGGRKDPEQAQIDKAEQIQLLSAAVREL-GRDKKKVSA-----
SstCas7-11 GTQRTLNRVDYATGKAHDFFKVWEIDHSLLSVFQGKISIADN-I----
GDGATKLLEDSLRFTDRLCGAICVISYDC--------------------------------------------
IENSDGKENGKTGEAAH----IMGESDAGKTDAENIANAIA-DMMGTAGEPEKLRILADAVRAL-
RIGKNTVSQ-----
CsbCas7-11 ASGRILNRVDFDTGKAKDYFRTWEADYETYGTYTGRITLRN--------
EHAKKLLLASLGFVDKLCGALCRIEVIK----------------------------------------
KSESPLPSDTKEQSYTKDDTV-----EVLSEDHNDELRKQAEVIV-EAFKQNDKLEKIRILADAIRTL-
RLHGEGVIEKDE--
CjcCas7-11 ASERILNRVDYTTGKAHDYFKVWEVDDDQWWQFTGTITMHDD------
CSKAKGLLLASLCFVDKLCGALCRIEVTG----------
NNSQDENKEYAHPDTGIITSLNLKYQNNSTIHQDAVPLSGSAHDNDEPPVH--
DNDSSLDNDTITLLSMKAKEIV-GAFRESGKIEKARTLADVIRAM-RLQKPDIWEK----
CbfCas7-11 CKERVLNRVDFRTGKAQDYFR VFEIDHEDWGVYTGEITITE--------
PRVQEMLEASLKFVDTLCGALCRIEIVG------------------------------
SADETKRTTSSKEGCPASTTTRDCSSSEND----DTSPEDPVREDLKKIAHVIA-
NAFQNSGNREKVHALADAIRAM-RLEESSIINT----
HvsCas7-11 APKRALNRIEQYTGKAQDYFTVYEALNKEFWTFKGRIRIKED-I----
YDKVTDLLFSALRCVEKIAGALCRIEIDK---------------------------------------------------EPSQQKGFV
K-------------RQLSKQAKEDIE-KIFQVVKDAQKLRLLSDCFRELTRMANKDELA-----
HsmCas7-11 GIRRILNRVDFTTGKAQDYFYVWEVEHSMCPKFQGTVKINED-M--
PKYNVVKDLLISSIQFVDKLCGALCVIEI-----------------------------------------------------GKTKNYI
C----QSFSSNIPEEEIKKLAQEIR-DILKGEDALDKMRVLADTVLQM-RTKGPEIVNE----
FmCas7-11 AQKRTSNRVDFFSKKAHDHYGVWEVTA--VKNLLGYIYISDA-
ITESHQKTVISLLKAALSFTDTLCGANCKLELSD----------------------------------------
EPVDSIHSNQSASNFNPHSGAAPSQCSQSMP----PFNMDQETKELANTLCKAFT-------
GNMRHLRTLADAVREM-RRMSPGISS-----
DpbaCas7-11 IRPRILNRMDPGTSKARDYFRVFEIENQLCSQFRGWIWLSGD-L--
PNMELVKSLLAAGLSNVATLAGAVCRIRIVSTDNPSMKQDLTTQDLIDDFTNYYLKGDTPPANLAAS
GKGDAFPRFSPGSGDHPDTTGVSHA----DMASSHEGTALAKDIAEKCK-
DIL SQISASEQLRRLADIMRDL-RQDSNREIMYRQ--
Domains
.........................................................................
.........................................................................
........................................................
SECTION 3 (SEQ ID NOS 611-631, continued from section above)
D429
WmCas7x3 ----------------------------------------------------------------------------------
---------------------------------MTTPSAPKSSL------PA----LHWLIRAELEVLTPLHLG-----------------
----TGTDQRITPDAPDA-----DPY
SER16298.1 --------------------------------------------------------------------------------
----------------------------------------MTDTV------KS----GRWIITGQFQLVTPMHIG----------------
------TGLDEEMDKQSGESVDKKQNNS
GwCas7x3 ----------------------------------------------------------------------------------
-----------------------------------------------MSNQ----TRWIIEGTLELITPLHIG------------------
----TGLDE-------KERDENKETR
PID64649.1 --------------------------------------------------------------------------------
-----------------------------MSDTQKQAIHENKWHF-----RGI----KRWEISAYLKTLSPLHIG----------------
------DG---GTIPVTIKDTQGKNREV
WP_031436019.1 ----------------------------------------------------------------------------
---------------------------------------------MNLPTWKL-----NNE----KRWHISIVLTTATPLHIG--------
--------------SG--EFCEHDDVKNNDG--EPV
HEB50754.1 --------------------------------------------------------------------------------
------------------------------------MAAVQDRWTLMDQQGNEL----KRFRITAELETASSLHIG---------------
-------AS--ETVEHDLIKNDDG--TPV
HreCas7-11 -LPAGKPEG--------RESIWEK------------TPTGE--TLTLRQLLKSANVPGE-------
SWRAFCEELGEQLYRLEKNLYS--HARPLPRLLGETEFYGQPARKS---DDPPMIRASYR----AFPS----
YVWVLDGILRAETPFYFG-----------------------TET-----------SEGQTS-
CmaCas7-11 -LPKGKKGG--------RTSIWNK------------KVADD---FTLRDCLKNQKIPNE-------
LWRQFCEGLGREVYKISKNISN--RSDAKPRLLGETEYAGLPLRKE---DEKEYSPTYQN----QESLPK--
TKWIISGELQAITPFYIG-----------------------HVN-----------KTSHTR-
HvmCas7-11 -LPKGKTDK--------
DNSVWDKPLKKDILPSPRMPASEDDDTPTLRKVLKDEINGQED------
MWRKFCEALGNSLYDLSKKAKERKRTEALPRLLGETEIYGLPMREN---KEDEPLPSSLT----YK------
FKWLIAGELRAETPFFFG-----------------------TEV-----------QEGQTS-
SmCas7x3 FLPPGRVNK---DGRRVPHYVWDIP-----------LGKGD----TLRKRLEFLAASCEGDQA--
-KWRNICESEGQALYEKSKKLKD--SPAAPGRHLGAAEQVRPPQPPV---SYSEESINSDL----PL------
AEWIITGTLRAETPFAIGM---------------------DAPI-----------DDDQTS-
oral_meta -LPMGSQGL---GGRL-PHHLWDVP-----------LVSKDRETQTLRSCLEKIAAQCKSEQT---
QFRLFCQKLGSSLFRINKGVYL--APNSKISP----EPCLDPSKTI---RTKGPVPGKQK----
HRFSLLPPFEWIITGTLKAQTPFFIP----------------------DEQG-----------SHDHTS-
DisCas7-11 -LPKDHDGK---D----DHYLWDIG-----------KKKKDENSVTIRQILTTSADTKELKNAG--
KWREFCEKLGEALYLKSKDMSG--GLKITRRILGDAEFHGKPDRLE---KSRSVSIGSVL------------
KETVVCGELVAKTPFFFG----------------------AIDE-----------DAKQTD-
DsbaCas7-11 -LPLNHEGK---E----DHYLWDK------------KAGGE----TIRTILKAAAEKEAVAN----
QWRQFCIELSEELYKEAKKAHG--GLEPARRIMGDAEFSDKSVPDT---VSHSIGISVE-------------
KETIIMGTLKAETPFFFG----------------------IESK-----------EKKQTD-
SstCas7-11 -LPLDHEGK---E----NHHLWD-------------IGEGK----SIRELLLEKAESLPSD-----
QWRKFCEDVGEILYLKSKDPTG--GLTVSQRILGDEAFWSKADRQL---NPSAVSIPVT-------------
TETLICGKLISETPFFFG----------------------TEIE-----------DAKHTN-
CsbCas7-11 -LPDGKEER---DK---GHHLWDI------------KVQGT----ALRTKLKELWQSNKDI-----
GWRKFTEMLGSNLYLIYKKETG--GVSTRFRILGDTEYYSKAHDSE---GSDLFIPVTPP----EGIET---
KEWIIVGRLKAATPFYFGVQQPSDSIPGKEK------KSEDSLV-----------INEHTS-
CjcCas7-11 -LPKGINDK---------HHLWDR------------EVNGK----KLRNILEELWRLMNKRN----
AWRTFCEVLGNELYRCYKEKTG--GIVLRFRTLGETEYYPEPEKTEPCLISDNSIPITPL----GGV-----
KEWIIIGRLKAETPFYFGVQSSFDSTQDDLDLVPDIVNTDEKLE-----------ANEQTS-
CbfCas7-11 -LPKGKSEKTTEQIEVNKHYLWDEI-----------PVNDT----
SVRHILIEQWRRWQSKKDD-PEWWKFCDFLGECLYKEYKKLTS--
GIQSRARVMGETEYYGALGMPD---KVIPLLKSDKT------------KEWILVGSLKAETPFFFGLET-----------
--------EQTE-----------EVEHTS-
HvsCas7-11 -LPLGPEDD---------GHYLWDKI----------KVEGK----TLRIFLRNCFSQYKD------
NWLCFCDEASKKGYQKYREKRH--KLTDRELPTATPKHFAEKKDPQ---ISPIYIDKDDK----V-------
YEWIIVGRLIAQTPFHFG-------------------------------------DEEKAE-
HsmCas7-11 -LPRGIEKK---G----GHWLWDKL-----------R---------LRKKFKEIANNYKD------
SWQELCEKLGNELYISYKELTG--GIAVKKRIIGETEYRKIPEQEI---SFLPSKAGYS-------------
YEWIILGKLISENPFFFG----------------------KETK-----------TEEQID-
FmCas7-11 -LPRGRLNK---EGEITAHYLWDE-------------RIDEK----
TIRQVLEDTIELSPARSIIYKNWISFCNQLGQKLYERAKD-ND--PILERKRPLGEAAFSKVPTSSH---
APRHDMNSRVK----GGFT----REWIIVGTLRALTPFYMG----------------------TGSQ-----------AGKQTS-
DpbaCas7-11 -VAEENHEK-------ASLLYKK------------TKKGD----SIAALIAGKTEGMDA-----
ETWRRLCEFLGQTFYGEAKEAGL--VETPVPRILGESERYSLQKKPT---VRTDLAAELVP----D-------
IEFIIKGNLIAETPFFFG----------------------TDIA-----------TETHTD-
Domains
.........................................................................
.........................................................................
........................................................
SECTION 4 (SEQ ID NOS 611-631, continued from section above)
*
G-rich loop
WmCas7x3 WQADIALDADGRPYLPGASLKGALKALARRRQVDAPCL-
PLFGDLNRGGAPHPDCIPPRRTRAGLAEFRDALQSHAT--QAD--GPDT---------
AQPRIAIDRITGSVVDKKLFHTQTVPVGTRFSVEI--ILRRADQ--NLAAQLVALLQHGPT-----
DPDFRLGAHANLGFGRVGL-YGNIDTRRFGPQQAQ-H
SER16298.1 WIQAIALDLNKKPYIPGASIKGALKALARRYYCASNL--NIFGDTI--
DTKDGDNKRKSVTVAGQAEFLNA-WYAAD--QED--KPFD---------
TITRVAIDRVTGTAEDRKLFNTRRVNPGVCFNYKI--IIQNACE--TEIQYLLDLLRKAAK-----
DPSFSLGAGANQSQGKVRC-LSSC-VRYFGKQEMH-D
GwCas7x3 WLEAVALDHKGQPYIPGASLKGALRALAKRHD---------FRNLF--DNKEVDGD-----
FVRQAEFLSA-WCVPD--TDK--GRL----------IQPRVAIDRVTGTAQDKKLFQTRLITPGTRFAMKI--
VVQNAVE--NEIADLLGLLNLLPD-----DPQFSLGAYANQGQGRVQW-FGKIQTRCFGINEAK-A
PID64649.1 EVNSVITGKAALPIIPGSTIKGRLRHYFSKHFSDKALLNKVFGEES--DATDDDQG-----
RGGLAEFHDAKWNPEK--
NRNLQGRYPYWNNTRKTYIEVSTAINRHTGAAKDKSLHHTECVPPGTVFEIKITGSMDDRCA--
ALVVAALEAIQTTGS-----RIFL--GAEDANGNGRIGL-TGKITVKQMDQAHII-Q
WP_031436019.1 KINACIKGSKGRPIIPGSTIKGKLYEWLKTRNTEENLLEKLFGKGH--
NSVSQDQG-----RGGKAEFHDAEIIEPL--TGS--
QPWPYWREEHQAFIAASTAIDRHKQVALQQSLHYMETVPAGIRFKFTFTGVMRDEEA--
ALLIAALDSFDKNON-----QPCF--GVDRANAYGRMEL-HGHLHVKVMGATEIS-S
HEB50754.1 QINALITGAGGLPIIPGSTIKGRFLARLRERGVDSALLETLFGKGH--DRETEDQG-----
RGGRAEFHDAPLCHRL--SGA--
RHFPYWRPERQIWVKAQTAVDRHRGTALRRSLRYTEMVPPGVRFRLTITGCMTDAEA--
DVLFALLEDLGDPRQ-----ACSF--GGAGADGNGTMRL-FGRPEVYCLDRSGIL-G
HreCas7-11 --QAIILCPDGSYRLPRSLLRGVIRRDLRAIL-GTGCNVSLGKVRP--CSCPVCE------
IMRRITVQQG---VSS--YRE--PAE----------VRQRIRSNPHTGTVEEGALFDLETGPQGMTFPFRL----
YFRTRSPYIDRALWLTINHWQE-----GKAI-FGGDIGVGMGRFRL-E-NLQIRSADLVSRR-D
CmaCas7-11 --STIFLNMNGQFCIPRSTLRGALRRDLRLVF-GDSCNTPVGS-RV--CYCQVCQ------
IMRCIKFEDA---LSD--VDS--PPE----------VRHRIRLNCHTGVVEEGALFDMETGFQGMIFPFRL----
YYESKNEIMSQHLYEVLNNWTN-----GQAF-FGGEAGTGFGRFKL-L-NNEVFLWEIDGEEED
HvmCas7-11 --ATILLNRDGYFRLPRSVIRGALRRDLRLVMGNDGCNMPIGG-QM--CECGVCR----
--VMRHIVIEDG---LSD--CKI--PPE----------VRHRIRLNCHTGTVEEGALFDMETGYQGMTFPFRL----
YCETENSDLDSYLWEVLNNWQN-----GQSL-FGGDTGTGFGRFEL-T-EPKVFLWNFSKKE-K
SmCas7x3 --SRTLVDRDGRYRLPRSTLRGILRRDLSLASGDQGCQVRLGPERP--CTCPVCL------
ILRQVVIADT---VSE--TTV--PAD----------IRQRIRRNPITGTAADGGLFDTERGPKGAGFPFSL----
RYRGH-APMPKALRTVLQWWSA-----GKCF-AGSDGGVGCGRFAL-D-NLEVYRWDLGTFA-F
oral_meta --RKILLTRDFYYRLPRSLLRGIIRRDLHEATDKGGCRVELAPDVP--CTCQVCR------
LLGRMLLADT---TST--TKV--APD----------MRHRVGVDRSCGIVRDGALFDTEYGIEGVCFPLEI----
RYRGN-KDLEGPIRQLLSWWQQ-----GLLF-LGGDFGIGKGRFRL-E-NMKIHRWDLRDES-A
DisCas7-11 --LQVLLTPDNKYRLPRSAVRGILRRDLQTYF-DSPCNAELGG-RP--CMCKTCR------
IMRGITVMDA---RSE--YNA--PPE----------IRHRTRINPFTGTVAEGALFNMEVAPEGIVFPFQL----
RYRGSEDGLPDALKTVLKWWAE-----GQAF-MSGAASTGKGRFRM-E-NAKYETLDLSDEN-Q
DsbaCas7-11 --LMLLLDGQNHYRIPRSALRGILRRDIRSVL-GTGCNAEVGG-RP--CLCPVCR------
IMKNITVMDT---RSS--TDT--LPE----------VRPRIRLNPFTGSVQEKALFNMEMGTEGIEFPFVL----
SYRGK-KTLPKELRNVLNWWTE-----GKAF-LGGAASTGKSIFQL-S-DIHAFSSDLSDET-A
SstCas7-11 --LKVLLDRQNRYRLPRSAIRGVLRRDLRTAFGGKGCNVELGG-RP--CLCDVCR------
IMRGITIMDA---RSE--YAE--PPE----------IRHRIRLNPYTGTVAEGALFDMELGPQGLSFDFIL----
RYRGKGKSIPKALRNVLKWWTK-----GQAF-LSGAASTGKGIFRL-D-DLKYISFDLSDKD-K
CsbCas7-11 --FNILLDKENRYRIPRSALRGALRRDLRTAF-GSGCNVSLGGQIL--CNCKVCI------
EMRRITLKDS---VSD--FSE--PPE----------IRYRIAKNPGTATVEDGSLFDIEVGPEGLTFPFVL----
RYRGH--KFPEQLSSVIRYWEENDGKNGMAW-LGGLDSTGKGRFAL-K-DIKIFEWDLNQ---K
CjcCas7-11 --FRILMDKKGRYRIPRSLIRGVLRRDLRTAFGGSGCIVELGRMIP--CDCKVCA------
IMRKITVMDS---RSE--NIE--LPD----------IRYRIRLNPYTATVDEGALFDMEIGPEGITFPFVF----
RYRGE-DALPRELWSVIRYWMD-----GMAW-LGGSGSTGKGRFAL-I-DIKVFEWDLCNEE-G
CbfCas7-11 --LRLVMDKKGRFRIPRSVLRGALRRDMRIAF-DSGCDVKLGSPLP--CDCSVCQ------
VMRSITIKDS---RSE--AGK--LPQ----------IRHRIRLNPFSGTVDEGALFDIEVAPEGVIFPFVM----
RYRGE--EFPPALLSVIRYWQD-----GKAW-LGGEGATGKGRFALAK-DLKMYEWKLEDK--S
HvsCas7-11 --GAILLTPDNRFRLPRTALRGILRRDLKLAG-ASACEVEVGRSEP--CPCDVCK------
IMRRVTLLDT---VSEDLRDF--LPE----------LRKRIRINPQSGTVAEGALFDTEVGPEGLSFPFVL----
RYKCE--KLPDSLTTVLCWWQE-----GLAF-LSGESATGKGRFRL-E-INGAFVWDLQK---G
HsmCas7-11 --MQILLTKDGRYRLPRSVLRGALRRDLRLVI-GSGCDVELGSKRP--CPCPVCR------
IMRRVTLKDA---RSD--YCK--PPE----------VRKRIRINPLTGTVQKGALFTMEVAPEGISFPFQL----
RFRGE-DKFHDALQNVLVWWKE-----GKLF-LGGGASTGKGRFKL-E-IEHVLKWDLKN---N
FmCas7-11 --MPTLQDSNDHFRLPRTALRGALRRDINQASDGMGCVVELGPHNL--CSCPVCQ----
--VLRQIRLLDT---KSK--FSM--PPA----------IRQKICKNPVLSIVNEGSLFDVELGIEGETFPFVM----
RYRGG-AKIPDTIITVLSWWKN-----ERLF-IGGESGTGRGRFVL-E-CPRIFCWDVEK---G
DpbaCas7-11 --LPILLTSDRHFRIPRSVLRGILRRDLRLVT-GSGCSVKLGRSEP--CACDVCQ------
IMRSLTMRDC---VSS--CKV--PPE----------IRHRIRLNPVTETVEEGALFDMEIGPQGISFPFVL----
RSRGVNSSFSTRLKNVLTWWSE-----GKIF-MGGDKGTGKGRFTL-A-ELEAYYFRLTTKRIG
Domains
.........................................................................
.........................................................................
........................................................
SECTION 5 (SEQ ID NOS 611-631, continued from section above)
D654
WmCas7x3 WF-----AAAQTQADARW-TDFAEA---VT-----------------LTAPAPAPAQP----------------
APHRLALP--------------------------------LSLTFHTPFLVKQPEHKH--R----KPQDNAPDGTPR----QRGD-----
-----RA----LLPGASLRGRLRSQAERILRTLGCK
SER16298.1 WF-----RAIQNGKQEHW-QIFAKP---SN-----------------IKYADLERPDI----------------
IANSLNLP--------------------------------LTLDFHTPFLVKASKKK--------DEAKNEADAKPR---TNHQG------
----QV----ILPASSLRGRLRAQAEKILRTMGQD
GwCas7x3 WY-----EEIRKDESKCW-TAFAKP---KN-----------------VSTP---PTPA----------------
KEAQLTLP--------------------------------LNLAFHTPFLVKQAGIK-----------ADDADAVPR---RTHDD--------
--KI----VLPASSLRGRLRTQSERILRTLGCE
PID64649.1 WL-----Q-----KDST--TCVASF---SN-----------------VKAENETQVKQ--------------
MVQRHIAPKLNSVVSAAG------------------PSYDITLHFDGPFVVNDSDKC-------KAEDTPDIYPL---
EEKNG----------VP----AFPVRSFRGAIRSQAERIIRTIGGQ
WP_031436019.1 WL-----NSFSE-NDKK--MAMESA---RN-----------------LEQQEINTLIE----------------
QGNALFKA------SCDE------------------VKLGLTLKFKGPFLVNDPYAVKILS----SNENAKTDHYPL---
LDKNR----------NP----YLPVSSFRGVLRSQAERIIRTLGGK
HEB50754.1 WL-----ASFEKGGNGG--MAMTAA---AL-----------------LQADTVQRRAD----------------
KVRQAWQP------PDVG------------------PRLHVELRFSGPFLVNDPSRN---T----PDITQAPDMVPL---
VDEDG----------NP----MLPASSFRGALRAQAERIIRTLGGR
HreCas7-11 FS-----LYLRA---RGL-KGLSR----EE-----------------VTRIGLNEEQWEAVMA----DD-----
PGTHYNPF------P--W------------------EKISYTLLIHSPLISNDPIAAM--L----D--
HDNKDAVMVQKTVLFVDESGN-----YSQMPHHFLKGSGIRGACRFLLGRK-------
CmaCas7-11 YL-----QYLFS---RGY-KGIE-----TD-----------------EIKKVADPIKWKTLF-----TK-----
LEIPPEKI------P--L------------------TQLNYTLTIDSPLISRDPIAAM--L----D--
NRNPDAVMVKKTILVYEQDSSTHKNVPKEVPKYFIKSETIRGLLRSIISRT-------
HvmCas7-11 HE-----AYLLN---RGF-KGQMPV---QD-----------------VKTKSFKTKTWFQIH-----RE----
-LDISPKKL------P--W------------------YSTDYRFNVTSPLISRDPIGAM--L----D--
PRNTDAIMVRKTVFCPDPNA---KNRPAPATVYMIKGESIRGILRSIVVR--------
SmCas7x3 RQ-----AYSEN---NGL-RSPEEEFDLAV-----------------IHELAEGLAKEDGQK------------
ILKGTEPF------TC-W------------------QERSWQFSFTGPLLQGDPLAAL--N------
SDTADIISFRRTVVDNGEVL------REP----VLRGEGLRGLLRTAVGRV-------
oral_meta RA-----DYVQK---CGLRRGVGDDTAINL-----------------EKDLSLNLPE----------------------
SGY------P--W------------------KKHAWKLSFQVPLLTADPIMAQ--T-------
RHEEDSVYFQKRIFTSDGRVV-----LVP----ALRGEGLRGLLRTAVSRA-------
DisCas7-11 RN-----DYLKN---WGW-RDEKGL---EE----------------LKKRLNSGLP-----------------
EPGNYRDP------K--W------------------HEINVSIEMASPFINGDPIRAA--V----D--
KRGTDVVTFVKYKAEGEEAK------PVC----AYKAESFRGVIRSAVARI-------
DsbaCas7-11 RE-----SYLSN---HGW-RGIMEN---SI-----------------VHESPLEGGAGGCSF------------
GLSDLPKL------G--WHAEDLKLSDIEKYKPFHRQKISVKITLNSPFLNGDPVRAL--T-------
EDVADIVSFKKYTQGGEK--------IIY----AYKSESFRGVVRTALGLR-------
SstCas7-11 RK-----DYLDN---YGW-RNRIEA---LS-----------------LEKMPLD--------------------
RMNDYAEP------L--W------------------QKVSVEIEIGSPFLNGDPIRAL--I----E--
KDGSDIVSFRKYADDSGK--------EVY----AYKAESFRGVVRAALARQ-------
CsbCas7-11 IN-----EYIKE---RGM-RGKEKELLEMG----------------ESSLPDGLIPYKFFEEREC-LF---
--PYKENLKP------Q--W------------------SEVQYTIEVGSPLLTADTISAL--T----E--
PGNRDAIAYKKRVYNDGNNA------IEPEPRFAVKSETHRGIFRTAVGRR-------
CjcCas7-11 LK-----AYICS---RGL-
RGIEKEVLLENKTIAEITNLFKTEEVKFFESYSKHIKQLCHECIINQISFLWGLRSYYEYLGP------L--W-
-----------------TEVKYEIKIASPLLSSDTISAL--L----N--KDNIDCIAYEKRKWENGGIK------FVP----
TIKGETIRGIVRMAVGKR-------
CbfCas7-11 LH-----AYIDT---YGH-RGNEHAIGTGQ-----------------
GIDGFRSGSLSDLLSDISKESFRDPLASYHNYLDK------R--W------------------
IKVGYQITIGAPLLSADPIGAL--L----D--PNNVDAIVFEKMKLDGDQVK------YLP----
AIKGETIRGIVRTALGKR-
HvsCas7-11 LF-----NYIKN---HGF-RGEERLFLEGN-----------------EAELEKMGIQINTELLQPEMIK--
---KEKNFTDF------P--Y------------------DLIKYQLNISSPLLLNDPIRAI--ALYEGE--
GKAPDAVFFKKYVFENGKIE------EKP----CFKAESIRGIFRTAVGRI-------
HsmCas7-11 FH-----SYLQY---KGL-RDKGDFNSIKE-----------------IEGLKVETEEFKVK---------------
---KPF------P--W------------------SCVEYTIFIESPFVSGDPVEAV--L----D--SSNTDLVTFKKYKLEESK-----
---EVF----AIKGESIRGVFRTAVGKN-------
FmCas7-11 QN-----DYIQY---HGF-RNKEDEL--LS-----------------VYSTVSGLAEKNDV-------------
NLNNARDF------S--F------------------DKICWEVQFDGPVLTGDPLAAL--F-------
HGNTDSVFYKKPILKSGEKE------PSY--QWAIKSDTVRGLIRSAFGKR-------
DpbaCas7-11 KNVWVIGNYLKS---QGW-RG-------AE-----------------LETHFDSLKEWKSL------SF----
-SDSDVKVF------T--W------------------HKITWKVSFEGPVLTNDPIAAD--I-------RNESDAVFYQKSV--
AGEKG------PVY----ALKGEGLRGIVSSSLCKK-------
Domains
.........................................................................
.........................................................................
........................................................
SECTION 6 (SEQ ID NOS 611-631, continued from section above)
*
WmCas7x3 VAQG--------HAVPPVKNNTCP-DPATL-----LFGTAGWRGLLRTDD---------------------
-------CTGTA---------------------------------------------------------------------------
--PAT----------------------------------LVD
SER16298.1 IPQG-------HAAPAYDGIAHR-DLISL-----
LFGTAGWKGIVCASDLIHSIPEYALQFNGVRETISDLSDTVKSCIIVDLVKTSTAAEKTEEQLHIRIVDS
AGSLIVHKSENSSWANDTFRDASVKDNFKARLKEIADPQDLSDALRADIKKRAFQLAT-----------------
-----------------LTR
GwCas7x3 TPQG--------HTAPAYRKGQPHDDLAVL-----LFGAAGWRGIVQTSD----------------------
------CIVED---------------------------------------------------------------------------
-KSIK----------------------------------TRR
PID64649.1 CCDG--------SINNTCK------NPKNLCIACEMFGSTGWKTSIEMDPFL----------------------
----CVDRE-----------------------------------------------------------------------------
LKP----------------------------------FII
WP_031436019.1 CCS-----------TDDPCKPIFDKGDLSKLCLACQIFGASGWKTVINIHDFK----------------
----------AINKS------------------------------------------------------------------------
-------K----------------------------------KTK
HEB50754.1 CCD----------TSSPCRPLGSSDKVGELCLACQVFGAPGWGTTLHIQGFT-----------------
--------CTSV---------------------------------------------------------------------------
----FRR.---------------------------------EQE
HreCas7-11 -DAPNE------NGLTYFE--ADHEE--CDCLLCSLFGSKHYQGKLRFEDAE------------------
--------LQDEV--------------------------------------------------------------------------
---EA-----------------------------------IK
CmaCas7-11 -EIKLE----DGKKERIFN--LDHED--CDCLQCRLFGNVHQQGILRFEDAE------------------
---------ITNKN-------------------------------------------------------------------------
----VS----------------------------------DCC
HvmCas7-11 -------------NEELYD--TDHED--CDCILCRLFGSIHQQGSLRFEDAE-------------------------
--VQNSV-----------------------------------------------------------------------------SD--
---------------------------------KK
SmCas7x3 ------------AGDDLLT--RSHQD--CKCEICQLFGSEHRAGILRFEDLP-------------------------
-PVSPT-----------------------------------------------------------------------------TV---
------------------------------ADKR
oral_meta ------------YGISLIN--DEHED--CDCPLCKIFGNEHHAGMLRFDDMV-------------------------
-PVGTW------------------------------------------------------------------------------ND-
---------------------------------KK
DisCas7-11 -HME-------DGVPLTE--LTHSD--CECLLCQIFGSEYEAGKIRFEDLV----------------------
----FESDP-----------------------------------------------------------------------------
EP-----------------------------------VT
DsbaCas7-11 -NQGNDDIT-GKKNVPLIA--LTHQD--CECMLCRFFGSEYEAGRLYFEDLT------------
-------------FESEP---------------------------------------------------------------------
---------EP---------------------------------RR
SstCas7-11 -HFDKEGKPLDKEGKPLLT--LIHQD--CECLICRLFGSEHETGRLRFEDLL---------------
---------FDPQP--------------------------------------------------------------------------
----EP----------------------------------MI
CsbCas7-11 ------------TGDLG-K--EDHED--CTCDMCIIFGNEHESSKIRFEDLE-------------------------
LINGN------------------------------------------------------------------------------EF---
---------------------------EKLEKH
CjcCas7-11 -------------SGDLG-M--DDHED--CSCTLCTIFGNEHEAGKLRFEDLE-----------------------
-VVEEK------------------------------------------------------------------------------
LPSEQNSDSNKIPFGPVQDGDGNREKECVTAVKSYKKKL
CbfCas7-11 ------------NNLLA-K--NDHDD--CTCSLCAIFGNENETGKIRFEDLE-----------------------
-VYDKD------------------------------------------------------------------------------IA--
------------------------------KK
HvsCas7-11 ------------KNVLT----KNHED--CICVLCHLFGNVHETGRLKFEDLK-----------------------
--IVSGQ-----------------------------------------------------------------------------EE--
--------------------------------KF
HsmCas7-11 ------------EGKLTTE--NEHED--CTCILCRLFGNEHETGKVRFEDLE-----------------------
---LINDS----------------------------------------------------------------------------AP--
---------------------------------KR
FmCas7-11 ------------DALLI----KSHED--CDCLLCEAFGSKHHEGKLRFEDLT-------------------------
PKSDE----------------------------------------------------------------------------IK----
-----------------------------TYR
DpbaCas7-11 ------------KNLSS----NLHED--CECLRCKIFGSKHQEGNIRFEDMT------------------------
-VSQES----------------------------------------------------------------------------EV----
----------------------------REKL
Domains
.........................................................................
.........................................................................
........................................................
SECTION 7 (SEQ ID NOS 611-631, continued from section above)
D745 D758 G-rich loop
WmCas7x3 HDMLAIDRFTGGGKDGAKFKLRYA-----ECPTLEGQLSLDLSRLRSARLD------
GANAADTPWIALGLLTLVLRDLAEGDIPFGHGSAKGYGRCRAQGLPDRWRQALEAHFGPNADARA
LAALRAWCRTHATAAL--DAPCSLAGSAPTPAAAAPS--GQAAPAD---AFHN--PYHFI-P-F-
SQPDIDRWLSPDA
SER16298.1 HEMVAIDRFTGGGKEGAKFNVDYI-----ECPTLTGAIYLDLHRLKQAQLK------
NDEDALKP--
ALGLISLLLRDLAEGDIAFGFGANKGYGQCREHAVLDNWEERLKKIGAGLTIDGALQALRDTVALEP
PAEF---PPEIEKTTDDNQPEAPDF--NLKPASN---GFHN--PYHFI-P-L-NNPKIGDWPEAKA
GwCas7x3 HEMLAIDRFTGGGKDGAKFNVDYV-----ECPTLAGKLSLDLARLKNAKLK------
GGKDALLP--
ALGLMTLMLRDLAEGDIPFGYGISKGYGQCRASSALGDWAELLKQHLGADSADTTVQALREYLGN
PKGQELKLDPPSADATQAGVPAQQNAAKTQAQGAQE---KFHN--PYHFI-P-L-SKPDISQWPEPQK
PID64649.1 QEFVAIDRFHGGGKDEAKFNAAHY-----QAPVFKGKVRVS-QRVGN-DIS------WRK--
-------GLLALIFRDLKEGDIYFGFGTNKGYGAVKKAEINPDGN-------
ASDFSESDIEAFINKCREKKGLYNC--NPIKKPGKTKVSKNLPPAIVPLDRTDS---KFYN--PYHFI-P-V-
KKPNTSSWAEKTA
WP_031436019.1 QDFVAIDRFHGGGKDGAKFDATHF-----ERPEFEGAISFS-PRMANNDLD------
WGK---------GLLALVLRDMQEGDMTFGYGANKGYGGLESASITGIEQ-----------
ITSDIQAFRDKCVASPQTWLCDEAVKPANQQD----KIPPA--GIQVANS---GFHN--PYQFI-P-S-
KEPDTGHWLPVLG
HEB50754.1 QTFVAIDRFHGGCKEGALYTIRHA-----ESPRFEGHLVID-PRMPA----------WGR-------
--GLLALVFRDLREGDITFGLGAGKGYGVVDAAVVQDMAE-----------
LEPYVEAFREQCRQHQGMADCHSAPSPQPLRDHDLAEIPPA---EEAPGE---TFLN--PYHFV-P-I-
REPDTGSWLARDE
HreCas7-11 CDHVAIDRFHGGTVHRMKYDDYPLPGSPNRPLRIKGNIWVK------RDLS-----DTEK--
-------EAVKDVLTELRDGLIPLGANGGAGYGRIQRLMI-DDGPGW------------------
LALPERKEDERPQPSFSPVS--------LGP-VHVNLKSGSDTADVYYYHPHYFL-E-PPSQTV---------
CmaCas7-11 IDHVAIDRFTGGGVEKMKFNDYPLSASPKNCLNLKGSIWIT------SALK-----DSEK---
------EALSKALSELKYGYASLGGLSAIGYGRVKELTLEEN----------------------
DIIQLTEITESNLNSQSRLS--------LKP-DVKKELSNN---HFYY--PHYFI-K-PAPKEV--------
HvmCas7-11 MDHVAIDRFTGGGVDQMKFDDYPLPGCPAQPLILEGKFWVK------DDID-----
DESK---------SALEKAFADFRDGLVSLGGLGAIGYGQIGDFELIGGSADW------------------
LNLPKPEENRTDVPCGDRSA--------QGP-EIKISLDAD---KIYH--PHFFL-K-PSDKNV---------
SmCas7x3 LDHVAIDRFDQSVVE--KYDDRPLVGSPKQPLVFKGCFWVQ------TSGMT----HQLT--
-------ELLAQAWRDIAAGHYPVGGKGGIGYGWINSLVVDGEK---------------------
ITCRPDGDSISLTTVTGDIP--------PRP-ALTPPAG-----AIYY--PHYFLPPNP-EHKP---------
oral_meta IDHVSCSRFDASVVN--KFDDRSLVGSPDSPLHFEGTFWLH------RDFQ-------ND---------
VEIKTALQDFADGLYSIGGKGGIGYGWLFDMEIPRSLRKLNSGFREA----------
SSIQDALLDSAKEIPLSAPLT--------FTP-VKG---------AVYN--PYYYL-PFP-AEKP---------
DisCas7-11 FDHVAIDRFTGGAADKKKFDDSPLPGSPARPLMLKGSFWIR------RDVLED---EEYC-
--------KALGKALADVNNGLYPLGGKSAIGYGQVKSLGIKGDDKRI------------------
SRLMNPAFDETDVAVPEKPK--------TDA-EVRIEAE-----KVYY--PHYFV-E-P-HKKV---------
DsbaCas7-11 FDHVAIDRFTGGAVNQKKFDDRSLVPGKEGFMTLIGCFWMR------KDKELS---
RNEI---------EELGKAFADIRDGLYPLGAKGSMGYGQVAELSIVDDEDSDDENNPAK----------
LLAESMKNASPSLGTPTSLKK--------KDA-GLSLRFDEN---ADYY--PYYFL-E-P-EKSV---------
SstCas7-11 FDHVAIDRFTGGAVDKKKFDDCSLPGTPGHPLTLKGCFWIR------
KELEKPDEDKSER---------EALSKALADIHNGLYPLGGKGAIGYGQVMNLKIKGAGD--------------------
VIKAALQSESSRMSASEPEH--------KKP-DSGLKLSFDDKKAVYY--PHYFL-K-PAAEEV---------
CsbCas7-11 IDHVAIDRFTGGALDKAKFDTYPLAGSPKKPLKLKGRFWIK------KGFS-----GDHK---
------LLITTALSDIRDGLYPLGSKGGVGYGWVAGISIDDNVPDDFKEMINK----------
TEMPLPEEVEESNNGPINNDY--------VHP-GHQSPKQDHKNKNIYY--PHYFL-D-S-GSKV---------
CjcCas7-11 IDHVAIDRFHGGAEDKMKFNTLPLAGSFEKPIILKGRFWIK------KDIV-----KDYK-----
----KKIEDAMVDIRDGLYPIGGKTGIGYGWVTDLTILNPQSGFQ-----------------
IPVKKDISPEPGTYSTYPSH--------STP-SLNKG-------HIYY--PHYFL-A-P-ANTV---------
CbfCas7-11 IDHVAIDRFTGGARDQMKFDTLPLIGSPERPLRLKGLFWMR------RDVS-----PDEK---
------ARILLAFLEIREGLYPIGGKTGSGYGWVSDLEFDGDAPEAFKEMNSK----------
RGKQASFKEKISFRYPSGAPK--------HIQ-NLKAT-------SFYY--PHYFL-E-P-GSKV---------
HvsCas7-11 FDHVAIDRFLGGAKEKYKFDDKPIIGAPDTPIVLEGKIWVK------KDIN-----DEAK----
-----ETLSQAFSDINTGIYYLGANGSIGYGWIEEVKA-LKAPSWLKIK-------------
EKPNFEKDTSLNISAIMNEF--------KKD-IQTLNLD-----KTYL--PYGFL-K-L-LEKV---------
HsmCas7-11 LDHVAIDRFTGGAKEQAKFDDSPLIGSPDSPLEFTGIVWVR------DDID-----EEEK----
-----KALKSAFLDIKSGYYPLGGKKGVGYGWVSNLKI-ESGPEWLRLEVQE----------
KSSQENVLSPVILSEVMDIEF--------NPP-KIDEN-------GVYF--PYAFL-R-P-LNEV---------
FmCas7-11 MDHVAIDRISGGAVDQCKYDDEPLVGTSKHPLVFKGMFWIN------RDSS-----
VEMQ---------RALIAAFKEIRDGLYPLGSNGGTGYGWISHLAI-TNGPDWLNLEEVP----------
LPQPTADIPVEECTAEPYPKF--------QKP-DLDQN-------AVYY--PHYFL-Q-P-GKPA---------
DpbaCas7-11 FDHVSIDRFTGGAANKLKFDDKPLVG---NPLVFQGVFWVH------QSIGNN---EKTQ-
--------EALSDAFKDVRDGLYPVGAKGSIGYGWIKGIEV-VEGPDWLK----------------
DALSAEKTVEAGIASEESEY--------KLP-DLPWISLLPKGRAIYN--PHYFL-GIP-KVTP---------
Domains
.........................................................................
.........................................................................
........................................................
SECTION 8 (SEQ ID NOS 611-631, continued from section above)
E959
WmCas7x3 H---RKTGGH-SRYRG--------------LSGRLVCALTTVTPLFVGAAA-RTP-ASDQHPK---
-P----VAGFALQNQPAIPATSLRGLLSSLFESISGSNLRVLHPT-PYSIRKTTKE---------------ALSA--I-
----------G--------------------------------
SER16298.1 ETLKANREGH-DQYHTGK------------FSGRIVCSLTTQTPLFIGAET-KPS-TSDREPS---
-------E----ARPFKLNGKHAIPATSLRGMLSSLFESVSNSNFRVLHPE-HYSVRKSLDDYV-------------
ALSA--M-----------------G--------------------------------
GwCas7x3 L--TEKGHSH-DRYAS--------------LSGRIVCRLTTQTPLFIGSEQ-TTP-TNPQAPK-------
---S----LHPFKLNNGLAIPATSLRGMISSLFESVSNSNFRVLDEK-TYSMRKTMQQ---------------SLSA--
M-----------------G--------------------------------
PID64649.1 FGTADSPHSH-GFYRKQTNE------QQPLYSGRLICMLTSETPFFIGAQA-ESD---
PTENE----------NQASLRHPYQLDGEPAIPSTSLRGLISTMTEAAANCAMRVLDSE-IISYRKPMNPSH----
---------ILSA--L-----------------G--------------------------------
WP_031436019.1 L-NADSHHSH-AFYRDQTDN------GEKL YHGRLICCLNTETPIFIGADK-KKD-
TEPAEIN----------N-------YRLNGELAIPATSLRGMISSLAEAASNSAMRVLDNG-LLSYRKTADD--------
-------ALRK--V-----------------G--------------------------------
HEB50754.1 L-DSSCCHSH-GFYRQQVDD-------RPLYHGRLTCLLETETPLFIGATG-DSS-V-
PSRIE----------N-------YRLGNRIAIPAASLRGMLSSLAEAASNSAMRVLHQG-ILSYRKKAKN-------------
--ALRE--I-----------------G--------------------------------
HreCas7-11 S-RELDIISHARTRDS---G------GEALLTGRILCRLITRGPIFIPDTN-NDNAFGLEGGI-G-
-----HKN----YRFFRINDELAIPGSELRGMVSSVYEALTNSCFRIMEEGRYLSRRMGADEFK--------------
-DFHP-----------------G--------------------------------
CmaCas7-11 V-RESRLISHVQGHDT---E------
GEFLLTGKIKCRLQTLGPLFIANNDKGDDYFELQHNNPG------HLN----
YAFFRINDHIAIPGASIRGMISSVFETLTHSCFRVMDDKKYLTRRVIPESETTQKRKSGRYQVEESDPD
LFP-----------------G--------------------------------
HvmCas7-11 Y-RERELVSHAKKKGP---D------
GKSLFTGKITCRLSTEGPVFIPDTDLGEDYFEMQASHKK------HKN----
YGFFRINGNVAIPGSSIRGMISSVFEALTNSCFRVFDQERYLSRSEKPDPTE--------------LTKYYP--------
---------G-------------------------------
SmCas7x3 K-RSDKIIGH-HTFAT---D-------PDSFTGRITCKLEVVTPLIVPDTE-G------EQPKDQ----
--HKN----FPFFKINDEIMLPGAPLWAAVSQVYEALTNSCFRVMKQKRFLSWRMEAEDYK----------------
-DFYP-----------------G--------------------------------
oral_meta E-RCLVPPSH-ARLQS---D---------RYTGCLTCELETVSPLLLPDTC--------REKDGN------
YKE----YPSFRLNNTPMIPGAGLRAAVSQVYEVLTNSCIRIMDQGQTLSWRMSTSEHK----------------
DYQP-----------------G--------------------------------
DisCas7-11 E-REEKPCGH-QKFHE---------G-RLTGKIRCKLITKTPLIVPDTS-
NDDFFRPADKEARKEKDEYHKS----
YAFFRLHKQIMIPGSELRGMVSSVYETVTNSCFRIFDETKRLSWRMDADHQN-------------VLQDFLP---
--------------G--------------------------------
DsbaCas7-11 H-RDPVPPGHEEAFRG---G---------LLTGRITCRLTVRTPLIVPNTE-
TDDAFNMKEKAGK-KKDAYHKS----
YRFFTLNRVPMIPGSEIRGMISSVFEALSNSCFRIFDEKYRLSWRMDADVKE---------------LEQFKP------
-----------G--------------------------------
SstCas7-11 N-RKPIPTGH-ETLNS---G---------LLTGKIRCRLTTRTPLIVPDTS-NDDFF--QTGVEG---
---HES----YAFFSVNGDIMLPGSEIRGMLSSVYEALTNSCFRVFDEGYRLSWRMEADRNV--------------
LMQFKP-----------------G--------------------------------
CsbCas7-11 Y-REKDIITH-EEFTE---E---------LLSGKINCKLETLTPLIIPDTS-DENGLKLQGNKPG--
----HKN----YKFFNINGELMIPGSELRGMLRTHFEALTKSCFAIFGEDSTLSWRMNADEKD---
YKIDSNSIRKMESQRNPKYRIPDELQKELRNSGNGLFNRLYTSERRFWSDVSNKFENSIDYKREILR
CjcCas7-11 H-REQEMIGH-EQFHK---E----QKGELLVSGKIVCTLKTVTPLIIPDTE-
NEDAFGLQNTYSG------HKN----
YQFFHINDEIMVPGSEIRGMISSVYEAITNSCFRVYDETKYITRRLSPEKKD--
ESNDKNKSQDDASQKIRK-----------------G--------------------------------
CbfCas7-11 I-REQKMIGH-EQYYE---SYPSGASGEKLLSGRIICSMTTHTPLIVPDTG------
VIKDPENK------HAT----YDFFQMNNAIMIPGSEIRGMISAVYEAMTNSCFRIFHEKQYLTRRISPEDKE-
-------------LREFIP-----------------G-------------------------------
HvsCas7-11 K-RTSSPITH-ERFYE---N---------HLTGFIECSLKVLSPLIIPDTE------TPEKEENG------
HKY----YHFLKIDNKPIIPGAEIRGAVSSIYEALTNSCFRVFGEKKVLSWRMEGKDAK----------------
EFMP-----------------G--------------------------------
HsmCas7-11 K-RTREPIGH-NEWKK---S---------LISGYLTCRLELLTPLIIPDTS--EEVIKEKVNNGE-
-----HPV----YKFFRLGGHLCIPAAEIRGMISSVYEALTNSCFRVFDEKRLISWRMTAEEAK---
RPDPKKSEEQNRMRFRP-----------------G--------------------------------
FmCas7-11 E-RERHPVSH-DHIDD---K---------LLTGRLVCTLTTKTPLIIPDTQ-
TNTMLPPNDAPEG------HKS----
FRFFRIDDEVLIPGSEIRGMVSTVFEALTGSCFRVINQKAHLSWRINADMAK----------------HYRP--------
---------G--------------------------------
DpbaCas7-11 E-REREPVGH-DRFQT---D---------LHTGRIICTLKTITPLIIPDTE-
NDKAFEVENASAD------HER----
FKFMRMGSQAAIPGSAIRSMTSSVFEALTNSCFRVLDQKSHLSWRMEADDAG----------------DYKP----
-------------G--------------------------------
Domains
.........................................................................
.........................................................................
........................................................
SECTION 9 (SEQ ID NOS 611-631, continued from section above)
D998
WmCas7x3 -----------RIVERNGELKLYPL---------------TLPTIHQNA-DNAY-----------PVPA----------
RWRKVF-YWESPV---------------------------------------------------------------------------
---------------------------------
SER16298.1 ----------RIVDDQGELKLQPL---------------TLPTLFGNR----N-----------NVPA----------
KWEKIFGTPSEDD----------------------------------------------------------------------------
--------------------------------
GwCas7x3 -----------RIVRHDQKLYLLPL---------------TLPTLPQGP-HGVY-----------DLGE----------
KWSAVF-DWQP-P---------------------------------------------------------------------------
---------------------------------
PID64649.1 -----------MVTKRGEDFWLIPL---------------AMPALSLNDEEHNY-----------KLDK---------
-RYRTMF--PDGLA---------------------------------------------------------------------------
---------------------------------
WP_031436019.1 -----------MVIYVDNKSFIIKL-------------------------------------------------
--------------NDAI-----------------------------------------------------------------------
-------------------------------------
HEB50754.1 -----------MIVLRDGKRFILPL----------------VPLM---------------------------------
--------------------EVT------------------------------------------------------------------
--------------------------------------------
HreCas7-11 -----------IV---VDGAKIREM---------------KRYRLPLYD-T--P-----------DKTS----------
RTKEMT-CPELFTRKDG-------------------------------------------------------------------------
-------------------------------
CmaCas7-11 -----------RVQKKGNKYKIEKM--------------DEIVRLPIYD-N--F-----------SLVE----------
RIREYH-YSEECASYVP-------------------------------------------------------------------------
-------------------------------
HvmCas7-11 -----------KVKRDGNKFFILKM--------------KDFFRLPLYD-F--------------DFEG----------
EAESLR-PNYDEDRNEEENKGKNKNTQ---------------------------------------------------------------
-------------------------------
SmCas7x3 -----------RV--LDGGKQIKKMG-------------DKAIRMPLYD-D--S-----------TATG----------
SIKDDQ-LISDCCPKSDE------------------------------------------------------------------------
-------------------------------
oral_meta -----------KI--TDNGRKIQPMG-------------KQAIRLPLYD-E--
VIHHVSTPGDTDDLEKLKAIVLELTRPWKELP-
EEQKKKRFEKCKNILDGRMLQQKELRALENSGFAYWRDKTSLTFDSFLKDAIEQEYPRYSGDYQRI
KALVVNITLPWKLLKKEERHKRFDKCRRILKGQQPLTKDERKALEESG
DisCas7-11 -----------RV--TADGKHIQKF--------------SETARVPFYD-K--TQKHFDILDEQEIAGE-----
-----KPVRMW-VKRFIKRLSLVDPAKHPQKKQDNKWKRRKEGI----------------------------------------------
------------------------------------
DsbaCas7-11 -----------RV--ADDGKRIEEM---------------KEIRYPFYD-R--T--YPER-----------
NAQNGY-F-RWDARISLTDNSMRKM-EKDG-VPRNVIYKLNTLKNKAYKSEKSFLFDL----------
-------------------------------------KNKAGGV
SstCas7-11 -----------RV--TDNGLRIEEM---------------KEYRYPFYD-R--D-----------CSDK----------
KSQEAY-FDEWERSITLTDDSLEKMAERKGDISPKDLKVLKSLKGKNYKSTEGLLAAF------------------
--------------------------------------KDKGGDT
CsbCas7-11 CAGRPKNYKGGIIRQRKDSLMAEEL---------------KVHRLPLYD-N--F-----------DIPD---
---------SAYK-ANDHCRKSATCSTSRGCRERFT------------------------------------------------------
--------------------------------------
CjcCas7-11 -----------LVKKTDEGFSIIEVERYSMKTKGGTKLVDKVYRLPLYD-S--E-----------
AVIA-------------SIQ-FEQYGEKNEKRNA--------------------------------------------------------
---------------------------------------------
CbfCas7-11 -----------IVRIINGDVYIEKA--------------EREYRLPLYD-D--V-----------HIIT----------
NYEELE-YEKYIKKNPGREQ----------------------------------------------------------------------
-------------------------------
HvsCas7-11 -----------RVSKKKGKLYMVKM---------------QALRLPVYD-N--P-----------ALAN-----------
-EIRSGS-IYE--------------------------------------------------------------------------------
-------------------------------
HsmCas7-11 -----------RIIKKDKKFYAQEM---------------LELRIPVYD-N--K-----------D-------------
KRNEIS-QNDPTRPS---------------------------------------------------------------------------
--------------------------------
FmCas7-11 -----------RIIQNNEKMFIQPY---------------KMFRLPFYA-G--F--------------------------
------------------------------------------------------------------------------------------
-----------------------------
DpbaCas7-11 -----------RFEKKDDKAVIRKF---------------KKKARFPFYA-G--P-----------------------
------------------------------------------------------------------------------------------
--------------------------------
Domains
.........................................................................
.........................................................................
........................................................
SECTION 10 (SEQ ID NOS 611-631, continued from section above)
WmCas7x3 --------------------PLRVY----FGS-R--------KQTYDS-R---QP-----HYLP-IQELS-
Y---------------LPND---SDCI------APDQGDLRFPSRDRDRKFLIGQCPISRYDCPIPETDLP------KLSPQERPRY----
-TR--GWVR--SLWTS-----NREK----ELPHTVK-H
SER16298.1 --------------------FLRIY----FDDIP--------SKFSSN-K---RY-----FYNCKATELKDF--------
-------IKSD---KYFIG---KRTPTV----FPKSSTEKSHLESLEFID--VEKFKKAVEN------LEITPGNNPY-----IH-
-GWVR--NLKDE-----FRE-----DIPDNVK-H
GwCas7x3 ---------------------PLRIY----FDPPP--------RRTYQS-Q---QP-----CYMK-LSTVK-Y---------
------SESN---PNQI----IAGENLGALRFPRGNQNTQFLIGQSNQD--ECPITQAEYA------QKSEDERNEY-----
TS--GWVR--TLVKP-----GR------DLPRSVK-H
PID64649.1 --------------------KLKVYLEKAYSNNV--------MKTFLN-N---EN-----TWTLAQSKIH-Y---
------------LPLT---PIQMQ---NGGINSYYNNLRTPSRSNNFLIGQTVAH--GNGIPASGPG------AGMVP-----
---------GILR--ILGKE-----HRQN----DLPQNKK-H
WP_031436019.1 ---------------------KLKQT----YTPGN--------MKDFIE-K---SN-----SWSPEHNTVY-Y---
------------LD-------------------------------------------NNQIPQESYM------NGMKP-------------
-GILR--ILGKE-----GREQ----EL-ENKL-H
HEB50754.1 --------------------KLRHA----YTDPA--------MKHFLD-D---KN-----SWSPRCNRVY-Y-----
----------LGRD-----------------------------------------GNQIPAETRG------AGMRP--------------
GILR--LLGRE-----GRHD----AL-QNKK-H
HreCas7-11 ---------------------RPERA----KKFNEEIAKVAVQNRAYLL-SLDEKERR---
EVLLGNREVT-FDECPDDEYSDDEYSELKYAQKYKDFIAVLKKNGQKRGYIKFTGPNTAN-------------
------KKN--------EDAPDKNY-----RS--DWDP--FKLNI------LLESDPECRVSN--IH
CmaCas7-11 --------------------SVKKA----IDYNRMLAQAADSNREFLY-NHPEAK-----
SILQGKKEVY-Y-ILHKQESKNRGKTKEINPNARYACLTDENTPGSRKGFIKFTGPDMVT-----------------
--VNK------ELKSKIAPIYDPEWEKDIPDWERSNQESNHKYSFILHNEIEMRSSQKK----
HvmCas7-11 -------------------KVKNA----VEFNIKMAGFAKHNRDFLK-KYKEQEIK---
DIFMGKKKVY-F-------TAGKHKPNEAHDNDKIALLTKGSNKKAEKGYFKFTGPGMVN------------------
-VKA------GVEGEECDFHIDESDPDV-YWNMS--------SILPHNQIKWRPSQKK----
SmCas7x3 --------------------KLQKA----LATNOKIALAAKHNQEYLA-QLSPDERE---
EALQGLKKVS-F--------WTESLANNEAPPFLIAKLGEERGKPKRAGYLKITGPNNAN-------------------
IAN-------TNNPDDGGY-----IP--SWKD--QFDYS-----FRLLGPPRCLPNTKGNR
oral_meta FANWHGRELLFDRFLKDENSCLIKA----ETTDRVIASVAKNNRDYLF-
EIKQQDFARYKRIIQGLERVP-F--------SLRSLAKSKETSFQIACLGLRRGRFLRKGYLKISGPNNAN-----
--------------VEI------SGGSHSNSGY-----SD--IWDD--PLDFS-----FRLSGKSELRPNTQKTR
DisCas7-11 --------------------ATFIE----QKNGSYYFNVVTNNGCTSF-
HLWHKPDNFDQEKLEGIQNGE-K-----LDCWVRDSRYQKAFQEIPENDP--
DGWECKEGYLHVVGPSKVE-------------------FSDKKGDVINNFQGTLPSV-----PN--DWKT--IRTND-----
------FKNRKRKNE--
DsbaCas7-11 -GRYKKLVLKHAEVRGGEIPYYSHP----TPTDCKLLSLVGPNRQLCR-
QDTLVQYRIIKHRRGAKPEED-F------MFVGTPSENQK-----GHKEN----NDHGGGYLKISGPNKIE--------
-----------KEN-------VLTSGVPSV-----PE--NMGA--VVHNCPP---RLVEVTVRCGRKQEEEC
SstCas7-11 GGNILGLIFKYAE-RIGDVPRYEHP----TDTDRMMLSLSEYNRNQ-K-
SDGKRAYKIIKPASKLGKGAY-F------MFAGTSVENKRICNPACTDKA----NKSVKGYLKISGPNKLE---
----------------KYN-------ISEPELDGV-----PE--DRNCQIIHNRI-----YLRKIFVANAKKR---K
CsbCas7-11 --------------------CGIKV----RDKNRVFLNAANNNRQYLN-
NIKKSNHDLYLQYLKGEKKIR-F--------NSKVITGSERSPIDVIAEL--NERGRQTGFIKLSGLNNSN-------
------------KSQ---------GNTGTTF-----NS--GWDR--FELNI-----LLDDLETRPSKSD----
CjcCas7-11 --------------------KIRAA----IKRNEVIAEVARKNLIFLR-SLTPEELK---KVLQGEILVK-F-
----------SLKSGKNPNDYLAELH---ENGTERGLIKFTGLNMVN-------------------IKN------V--NEEDKDF---
--ND--TWDW--EKLNI-----FHNAHEKRNSLKQ----
CbfCas7-11 --------------------KIKNA----HRFNKNIARIAESNRNYLC-SLDRAVRR---EILSGRKKVN-
F--------RLVKVNDNKNPDKEAVELC---KTGPLEGLVKFSGLNAVN-------------------ISN------
LRPGTAEEGF-----DA--KWDM--WSLNI-----ILNRMDVRNSQKK----
HvsCas7-11 --------------------KYKN-----SKVEIIFFQTVEGIRKFLRGNFNNVEWKK--
VLVTGIDPLA-I----------LPSQKIPGNDKWVKNLQ--SKISPVRGYFKFTGPNKIE-------------------
TKRREEEKDEKLRTKANKVSCLQ-KD--KWYE--AMHNHVE---YKQDYTPPNSPKTEP-L
HsmCas7-11 -------------------EYNHP----TEPERIFFSNAEKIRNFLK-RNS--------NYLHGSTPLL-F----
--ROWSISNRYDKIA----------LIGNKSQGHLKFTGPNKIE-------------------VSE---------
GTKCPKYETIPGRD--EWDK--AVHNYVEPGKFVTVISRKKGQKPKA-V
FmCas7-11 ----------------------------------------DPRNCLSE------------KQLLGIEPVK-L--------
WVKDFVASLVKPQTDIDIE----WKEKIGFVRVTGPNKVE-------------------VDS-----SNTPDPSLPEC-----
ES--DWKD--IHITE--------DGSTPSKNDR----
DpbaCas7-11 ----------------------------------------DTREAFTS------------DQIMGKEKVT-L--------
WVKDFEASLTVPDE-------IGWKKKRGYLKVTGPNKVE-------------------IDT------ENISENNPSP-----PD-
-SWQD--VRIND--------DGTIPDKKNR----
Domains
.........................................................................
.........................................................................
........................................................
SECTION 11 (SEQ ID NOS 611-631, continued from section above)
WmCas7x3 QLFI--PDPVE--TPAA--DDL------------------------------LPIP-QGV-
LDTFHALADLALAGQH----W-GKDE-TPADD-QLLPFTPAGRQR----HDA---
DRPPRDADRQTRLQPGDLVCFD--LG-------D-------DGAVSEISFSSIWREGLRLAG-KPN-
LATTADLLAQ----------V
SER16298.1 HVFL--PDTTKRVSP--------------------------------LEIP-PHV-KKRFHELADLALAGLH---
-L-KQGE-TIASPYKILPYTPIGRNK----LENHIHRVPNDLTCYMTRLKKGDLVFFD--VD-------N-------
DGQITEISFSSIWRAGI---GTKNK-LQTTADLLSQ----------R
GwCas7x3 HVFL--PDVFIDAPPPV--NDL--------------------------YPIP-DSV-
IQRFHDLADQVLASMN----L-KPEE-IVDST-NLLPYTPVGRRS----DSD----------CRDTRLQAGDLIFFD--
IDPPLHPG-E-------KSQITEISFSSIWRSGI---G-KDH-LLTTPDLLTN----------F
PID64649.1 ELFI--PVPDAFVADPK--TFLD---------------------TATAFLIP-RNV-
IDAFEKIAEKQTQSQKQDK-L-KHDE-------ERLPFHLKGTRR----EQN-----------HTLQIKTGDLVYFR--
PN-----A-K-------GDEVEEIAFSSIWRGKT---------SGTTADFFPD----------K
WP_031436019.1 ELFI--PVPLEYVDTEN--NKFDYQAYKK-------A------FLYRAIEIP-EPV-
LKRYSELADQRTMSQKSNKEL-KKDD-TCQSV-GWLPFHLKGTKR----QLD------
DKHNVGKLQIDEYDLIYYE--AS---------------GKEVTEVAFSSIWRGRV---ETNSS-QANKVYSFIP--------
--G
HEB50754.1 EYFI--EIPERYVD-QD--HCFDYRMFIR-------D------
RARNGTLVPISPVAWERYHCLAEERTLSQKNDPEL-REDK-ACASL-KWLPFHPKGRVR----ERD------
PENDVCHLSLRHGDLVFYA--EQ---------------NRVVSEISFSAIWRSRV---ETSDSYQAVTVDCFVP------
----K
HreCas7-11 CYPR--PLLVC----IK--DKAEYRIHKRCEAIFCSI-----GSPSDLYDIP-QKV-SNQYRTIL--
--QDYNDNTGK--IVE-IF------------RTQ----IKH-----------DQ--LTTGDLVYFK--PA-------A-------
NGQVNAVIPVSISRKTD--------ENPLAKRFKN----------D
CmaCas7-11 KYPR--PVFIC----KK--NGVEYRMQKRCERIFDFT---KEEEKDKEIVIP-QKV-
VSQYNAIL----KDNKENTET--IPG-LF-------------NSK----MVN-----------KE--LEDGDLVYFK--YK---------
------EGKVTELTPVAISRKTD---------NKPMGKRFPKISINGKMKPND
HvmCas7-11 EYPR--PVLKC----VK--DGTEYVMLKRSEHVFAEA------SSEDSYPVP-GKV-
RKQFNSIS----RDNVQNTDH--LSS-MF-------------QSR----RLH----------DE--LSHGDLVYFR--HD-------E-
------KRKVTDIAYVRVSRTVD----------DRPMGKRFKN---------E
SmCas7x3 EYPR--PGFTC----VI--DGKEYSLTKRCERIFEDI---SGGENQVVRAVT-ERV-
REQYREIL----ASYRANAAG--IAE-GF-------------RTR----MYD-----------TE-ELRENDLVYFKTAKQ-----
ADG-------KERVVAISPVCISREAD---------DRPLGKRLP-----------A
oral_meta EYPR--PSFTC----TV--DGKQYTVNKRCERVFEDS-------AAPAIELP-RMV-REGYKGIL-
---TDYEQNAKH--IPQ-GF-------------QTR----FSS-----------YR-ELNDGDLVYYK--TD-------S-------
QGRVTDLAPVCLSRLAD---------DRPLGKRLP-----------E
DisCas7-11 -----PVFCC----ED-DKGNYYTMAKYCETFFFDL------KENEEYEIP-EKA-RIKYKELL--
--RVYNNNPQA--VPESVF------------QSR---VARE-----------NVEKLKSGDLVYFK--HN---------------
EKYVEDIVPVRISRTVD--------DRMIGKRMS-----------A
DsbaCas7-11 KRKRLVPEYVC----ADPEKKVTYTMTKRCERIFLEK-------SRRIIPFT-NDA-
VDKFEILV----KEYRRNAEQQDTPE-AF------------QTI----LPE-----------NG-TVNPGDLLYFR--EE----------
------KGKAAEIVPVRISRKVD---------DRHIGKRID----------P
SstCas7-11 ERDRLVGEFAC----YDPEKKVTYSMTKRCERIFIKD-------RGRTLPIT-HEA-
SELFEILV----QEYRENAKRQDTPE-VF-------------QTL----LPD-----------NG-RLNPGDLVYFR--EE---------
-------KGKTVEIIPVRISRKID---------DSPIGKRLR-----------E
CsbCas7-11 -YPR--PRLLF----TK--DQYEYNITKRCERVFEID-----KGNKTGYPVD-DQI-
KKNYEDIL----DSYDGIKDQ-EVAE-RF-------------DTF----TRG-----------SK--LKVGDLVYFH--ID-------
G-------DNKIDSLIPVRISRKCA---------SKTLGGKLD-----------K
CjcCas7-11 GYPR--PVLKF----IK--DRVEYTIPKRCERIFCIP-----VKNTIEYKVS-SKV-CKQYKDVL-
---SDYEKNFGH--INK-IF-------------TTK----IQK-----------RE--LTDGDLVYFI--PN-----EGA-------
DKTVQAIMPVPLSRITD---------SRTLGERLPH----------K
CbfCas7-11 EYPR--PALHF----NH--DGKEYTIPKRCERVFVRAEAGKRAETEGSYKVP-RKV-
QEQYQNIL----RDYESNIGH--IDN-TF-------------RTL----IEN-------CG--LNNGSLVYFK--PD-----N-S--
-----RKEVVAITPVKISRKTD---------RLPQGDRFPHTS--------S
HvsCas7-11 ERPRNIPCFVC----SD--KEKIYRMTKRCERVFVSL-----GENAPKYEIP-ISA-
IKRYEVIL----SAYRENWERNKTPE-LF-------------RTR----LPG-----------DGRTLNEDDLVYFR--AD-------
E-------NEKVKDIIPVCISRIVD---------EVPLIKRLS-----------Q
HsmCas7-11 QRRRNVPAFCC----YDYNTNRCFVMNKRCERVFKVS------RDKPKYEIP-PDA-
IRRYEHVL----RKYRENWERYDIPE-VF-------------RTR----LPG-----------DGETLNEGDLVYFR--LD------
-E-------NNRVLDIIPVSISRISD---------TQYLGRRLP-----------D
FmCas7-11 -------VYRC----QL--KGVTYTVAKWCEAFWVKD------EGKKPITVN-AEA-
INRYHLIM----KSYQDNPQS--PPI-IF-------------RSLPVLNYKQ-----------DQKII--GSMIFYR--ES-----
AKS-------DKIVNEIIPVKISRTAD---------TELLAKHLPN----------N
DpbaCas7-11 -------KFIC----QY--GTTTYTVDKWCEAFFCDE-------EKDPYELA-PDV-ERKYRLLM-
---DSYHNNPQA--PPQ-IF-------------RSLPLFSETG---------PKKTLEHGDLVYFRLSEV-----
NKQSQSKKQVRERVTDIVPVSISRIAN---------NQPIGKHIA---------A
Domains
.........................................................................
.........................................................................
........................................................
SECTION 12 (SEQ ID NOS 611-631, continued from section above)
WmCas7x3 SPHLLPLG--------------------------MPGRS-A-----RLSPVEQLFGVVEYRPPQTAK--
GTRKPTDAPAAYALAGKLQVGFGRP---ARPF-----------ER-------EP-AVTLKELSTPKPPSPALYFRPKA-
-GDGYVSKAKLASQ--------------PQ----DYAPAGRKHYLH--ALRR
SER16298.1 DPNLVQLG--------------------------MGVRT-
KNTDRFKLSPAERLFGVVEHRDDDNTTVENVNQPNDKAQAFAFAGKVRIGFGLP---DKKT-----------
TV-----NGVS-PVTLKELSSPKPPSPAFYLKRKN--NDDFVSKKVAAEC--------------SE----
TMTLRGRKCYLH--AWRE
GwCas7x3 DVNLQPHG--------------------------MPGRT-Q-----SLSPAELLFGLVGTQNDQATT--------
------AYAGKVRIGFGLP---EEGH-----------NP-----RLDA-RITLKELSSPKPPSPALYFRKKSG-
KDEYVSKANLADK--------------PE----DYILRGRKMYLH--AWRK
PID64649.1 E--LLPFN--------------------------RNRS--------RVSPAELLFGFTENNPKEMKI------D----
RGLAFAGKIRISAGTL---SDKF-----------SDTTESDLFEP-ETTLKALSSPKPPSPALYFKEKKS-
GTQYIKKQDL--N--------------PG----KHEIQGRKIYLH--ALRN
WP_031436019.1 E--LLPFN--------------------------ESRK--------KVSPAELLFGFTQINKDGSKA------DD-
--KAQAFAGKVRISAGTI---SEYP-----------EN--EANLLEQ-
EVTLKALSTPKLPSPALYFRTINGNGSAYISKQEL--E--------------PS----KHLAKGRKYYLH--ALRT
HEB50754.1 E--LRPFN--------------------------RDRR--------AISPAELLFGFVELDESEHST------
EKSRYEQMAFAGKVRLSAGLP---VEDV-----------ED---SALLEPKPIVLKALSSPKPPSPPFYFVMRDG-
SGAYIAKKDL--S--PD----------------RHRIKGRKHYLH--GLRQ
HreCas7-11 S--LRPCA-GLCVE----DCNECPARCKK-VADYFNPHP-R-----GLCPACHLFG---------
TT--------------FYKGRVRFGFAWLTGEDGAPRWYKGPD----PC--DSG-KGR-PMTIPLLERPRPT-----
WSIPD--------------N--------------------SFDIPGRKFYVHHP-YSV
CmaCas7-11 S--LRSCS-HTCTE----DCDDCPNLCES-VKDYFKPHP-D-----GLCPACHLFG---------
TT--------------FYKSRLSFGLAWL---ENNAKWYISNDFQQKDS--KKE-KGG-KLTLPLLERPRPT-----
WSMPN--------------N--------------------NAEVPGRKFYVHHP-WSV
HvmCas7-11 S--LRPCN-HVCVE----GCDECPDRCKE-LEDYFSPHP-E-----GLCPACHLFG---------
TT--------------DYKGRVSFGLGWH--ESNTPKWYMP------ED--NSQ-KGS-HLTLPLLERPRPT-----
WSMPN--------------K--------------------KSEIPGRKFYVHHP-WSV
SmCas7x3 G--FQPCS-HVCLE----DCNTCSAKNCP-VPLYREGWPVN-----GLCPACRLFG---------
AQ--------------MYKGRVNFGFARL------P-----------DD--KQP-ETK-TLTLPLLERPRPT-----WVLPK--------
------SV-------------KGSNTEDATIPGRKFYLRHDGWRI
oral_meta E--YRPCA-HVCLE----ECDPCTGKDCP-VPIYREGYPAR-----GFCPACQLFG---------TQ--
------------MYKGRVRFSFGVP---VN-------------ST--RSP-QLK-YVTLPSQERPRPT-----WVLPE--------------
S-----------------CKGKEKDVPGRKFYLRHDGWRE
DisCas7-11 D--LRPCH-GDWVE----DGDLSALNAYP-EKRLLLRHP-K-----GLCPACRLFG---------
TG---------------SYKGRVRFGFASL---ENDPEWLIPGK---NPG--DPF-HGG-PVMLSLLERPRPT-----
WSIPG---------------S--------------DN----KFKVPGRKFYVHHHAWKT
DsbaCas7-11 E--LRPCH-GEWIE----DGDLSKLDAYPAEKKLLTRHP-K-----GLCPACRVFG---------
TG---------------SYKSRVRFGFAAL---KGTPKWLKED-----PA--EPS-QGK-GITLPLLERPRPT-----
WAVLH---------------ND-------------KE----NSEIPGRKFYVHHNGWKG
SstCas7-11 D--LRPCH-GEWIE----GDDLSQLSEYP-EKKLFTRNT-E-----GLCPACRLFG---------TG--
------------AYKGRLRFGFAKL---ENDPKWLMKN-----SD--GPS-HGG-PLTLPLLERPRPT-----WSMPD----
-----------DTLNRLKKDGKQEPKKQKGKKGPQVPGRKFYVHHDGWKE
CsbCas7-11 A--LHPCT-GLS-----------------------------D-----GLCPGCHLFG---------TT--------------
DYKGRVKFGFAKY---ENGPEWLI-------TR--GNN-PER-SLTLGVLESPRPA-----FSIPD---------------D------
--------------ESEIPGRKFYLHHNGWRI
CjcCas7-11 N--LLPCV-HEVN-----EGLLSGILDSL-DKKLLSIHP-E-----GLCPTCRLFG----------TT-----
---------YYKGRVRFGFANL---MNKPKWLT-------ER--ENG-CGG-YVTLPLLERPRLT-----WSVPS---------
-----D--------------------KCDVPGRKFYIHHNGWQE
CbfCas7-11 D--LRPCV-RDCLDT---EGDIRMLENSP-FKRLFHIHP-E-----GLCPACQLFG---------TT--
------------NYRGRVRFGFASL---SDGPKWFR-------KD--EGN-ETC-HITLPLLERPRPT-----WSMPD--------
------D--------------------TSTIPGRKFYVHHMGYET
HvsCas7-11 E--LWPCVLAECPLLGF-ECKKCELEGLP-EKIWFRINK-D-----GLCPACRLFG---------
TQ--------------IYKSRVRFSFAYA------KNW--------------KF-YDG-YITLPRLESPRAT-----WLILK-----------
---E--------------KDKHYIKYKVCGRKFYLHNSTYED
HsmCas7-11 H--LRSCV-RECLYEGWGDCKPCKLSLFP-EKMWIRINP-E-----GLCPACHLFG---------
TQ--------------VYKGRVRFGFARA-----GSNW--------------KF-REE-QLTLPRFETPRPT-----WVIPK---------
-----R--------------KD----EYQIPGRKFYLHHNGWEE
FmCas7-11 D--FLPCA-ATCLN----ECDTCNAKTCKFLPLYREGYPVN-----GLCPSCHLFG---------
TT--------------GYQGRVRFGFAKM---NGNAKFCQ-------GG--ERP-EDR-AVTLPLQERPKLT-----
WVMPN--------------E--------------------NSTIPGRKFFLHHQGWKK
DpbaCas7-11 A--FRPCA-YVCIE----ECEPCDAKTCP-IPVYREGYPIK-----GLCRACHLFG---------TT-
-------------GYKGRVRFSFAKL---NGDAVW---------AK--GAG-GKD-YFTLPLLEKPRPT-----WTMPN-----
---------E--------------------GAKIPGRKFYVHHNEWKT
Domains
.........................................................................
.........................................................................
........................................................
SECTION 13 (SEQ ID NOS 611-631, continued from section above)
WmCas7x3 QG---QVARLDNSGHVPSDGSGRPPW------QSRFDG--------QEDSGNK----
RRVRVEPIPAGETFHFEIDFDNLSPTELEQLCATLLP-HPAFEHRLGMGKPIGLGSVKLAVEGL---
LLVDRPRRYAEDEPNAPRHHRGW--RAN--ADAG--WPDHLQGDSPAAPLEAT-EQP-AALAERAMA---
------
SER16298.1 QNG--NVMKLDAIGVNSGGSTCKPPWKTHKPAANDQKE---------
FEEDKNKFITSRQVKIAPISENTPFYFEIDFNNLDATELAQLCATLQP-
APKFEHRLGMGKPLGLGSVKIEPVGL---FLINRHQRYTTDSTNCDRYHYAW--LKG--
EHAAWDWPEYFRQNVVTADCTQTFNDTFDKLVQNGLA---------
GwCas7x3 NE---QVVELSDTGH---DGGVRPPW------VSKFDE--------SADEGNK----
RRVSIEPIAKDESFYFEVDFHNLSRTELAQLCATLYP-NEKFEHRLGMGKPLGLGSIKITPLSL---
FLVNRSQRYATDGLDKPRYHAVW--HTG--TASEPRWPDHLQREQQGIAFEGVSTAP-TVMSLAAEA--
PID64649.1 ENN-QNVQRITSQGKFDNAANRTQPW----------VS--------QNEERNH----
LKTKCKPLKSGLNFFFHIDFNNLTQWELGLLCYALRP-CETFRHKIGMGKPIGLGTVKIDIAVL---
QTIDRYARYTDTTQDSERYNQGA--WIS--QELQNEIPN--QYKGKGISNKKGMLSP-EDCRKVEME------
---
WP_031436019.1 GDN--KVQKLGSQGETANGGDSKLPW---------VTH--------NPDERPQ----
LKVKIKPIKAE--FIFSLDFNNLTEWELGLLCYALRP-TDSFRHRIGMGKPLGLGSVKIDIMAL---
QTINRQQRYAQDGLEENRFNRHN--WVN--PPHQPRL----EKAGYSISLSSTPLNP-EILRATFTK---------
HEB50754.1 RGNPDRVQSLDRYGH-ATETAANPPW---------ETC--------HPEERPQ----
IKVRVQPVRRKTKFFFHLDFSNLSRWELGLLCYVLRP-TACFRHKLGMGKPLGLGSVRIDIASL---
QLIDRVRRYGTDDLTAGRYNMGG--HFN--ASCLDLLP---QQDSPAPDDSGAAPDP-GTLRQDFVK-----
----
HreCas7-11 ----D--------GI-------------------------------DGETRTP----
NNRTIEPLAEGNEFVFDIDFENLRDWELGLLLYSLEL-EDSLAHKLGLGKPLGFGTVQINIRGI---
SLKN---------------GSKG--WDTKTGDDKNQWIK----KGF------------AHLGIDIKE---------
CmaCas7-11 ENI-K--------NNQGN------------------QKDISLKPDSDAIKIKE----
NNRTIEPLGKDNVFNFEISFNNLRDWELGLLLYAIEL-EDHLAHKLGMAKAFGMGSVKIEIKNL---
LIKG---------------SIND--------ISKAELIK----KGF------------KKLGIDSLEKD-------
HvmCas7-11 DKI-R--------NRQFD------------------PAK-EKQPD-DVIKPNE----
NNRTVEPLGKGNEFTFEVRFNNLREWELGLLLYSLEL-EDNMAHKLGMGKALGMGSARIKAEAI---
ELRC---------------ESA---GQNAELKDKAAFVR----KGF------------EFLEIDKPGEN-------
SmCas7x3 -VM-A--------GTNPI------------------TG--------ESIEKTA----
NNATVEAIMPGATFTFDIVCENLDQQELGLLLYSLEL-EEGMSHTLGRGKPLGFGNVRIKVEKI---
EKRL---------------SDG---------SRREMIPP----KGA------------GLFMTDKVQDALRGLTEG
oral_meta MWG-D--------DDKPD----------------------------SRPSSEE----
CQDIIEGIGPGEKFHFRVAFENLDKNELGRLLYSLEL-DAGMNHHLGRGKAFGFGQVKIRVTKL---
ERRL---------------EPGQ--WRS--EKICTDLPV----TSS------------ELVISSLKK---------
DisCas7-11 -IK-D--------GNHPT------------------TG--------KAIEQSP----
NNRTVEALAGGNSFSFEIAFENLKEWELGLLIHSLQL-EKGLAHKLGMAKSMGFGSVEIDVESV---
RLRK---------------DWKQ--WRN-GNSEIPNWLG----KGF------------AKLKEWFRD---------
DsbaCas7-11 -IS-E--------GIHPI------------------SG--------ENIEPDE----
NNRTVEVLDKGNRFVFELSFENLEPRELGLLIHSLQL-EKGLAHKLGMAKSMGFGSVEIDVESV---
RVKH---------------RSGE--WDYKDGETVDGWIE----EGK------------RGVAAKGKA---------
SstCas7-11 -IN-C--------GCHPT------------------TK--------ENIVQNQ----
NNRTVEPLDKGNTFSFEICFENLEPYELGLLLYTLEL-EKGLAHKLGMAKPMGFGSIDIEVENV---
SLRT---------------DSGQ--WKD-ANEQISEWTD----KGK------------KDAGKWFKT---------
CsbCas7-11 -IR-Q--------KQLEI----------------------------RETVQPE----
RNVTTEVMDKGNVFSFDVRFENLREWELGLLLQSLDP-
GKNIAHKLGKGKPYGFGSVKIKIDSLHTFKINS---------------NNDK--IKRVPQSDIREYIN----KGY-------
-----QKLIEWSGNNSIQKGNVL
CjcCas7-11 -VL-R--------NN------------------------------DITPKTE----
NNRTVEPLAADNRFTFDVYFENLREWELGLLCYCLEL-
EPGMGHKLGMGKPMGFGSVKIAIERLQTFTVHQ--------------DGIN--WKP-SENEIGVYVQ----KGR----
--------EKLVEWFTPSAPHKNM--
CbfCas7-11 -VK-K--------NQ------------------------------RTLVKTE----
NNRTVKALDKENEFTFEVFFENLREWELGLLLHCLEL-
EPEMGHKLGMGKPLGFGSVKIRIDKLQKCVVNV--------------KDGCVLWEP-EEDKIQHYIA----KGL--
----------GKLTTWFGK---------
HvsCas7-11 IIN-N--------SKK-----------------------------EKEKKTE----NNASFEVLKEG-
EFTFKVYFENLENWELGLLLLSLT--GLGEAIKIGHAKPLGFGSVKIEAKKI---YFRE--------------
EAGK--FHP--CEKADEYLK----KGL------------NKLTSWFGK---------
HsmCas7-11 IYK-K--------NKK------------------------------NEIKKEK----NNATFEVLKQG-
TFYFKVFFENLELWELGLLIFSAELGGEEFAHKLGHGKALGFGSVKISVDKI---ILRR---------------
DPGQ--FEQRGQKFKRDAVD----KGF------------CVLENRFGK---------
FmCas7-11 IVD-E--------GKNPI------------------NG--------DVIEPDA----
NNRTVEPLAAGNDFSFEVFFENLREWELGLLRYTLEL-ESELAHKLGMGKAFGFGSVKIKIKSV---
DLRK---------------QGE--------------WEK----ATN------------TLVSEDKKS---------
DpbaCas7-11 -VQ-E--------GKNPI------------------DQ--------KAIRPNP----
NNSSVEVLNLGNEFQFEVSFENLEEWELGLLLYCLEL-EPGLAHKLGRGKAFGFGSIEAEVSKI---
EMRI---------------KSGT--WKNE-TSGKEKFIQ----SGL------------SQVPSFFKQDEK------
Domains
.........................................................................
.........................................................................
........................................................
SECTION 14 (SEQ ID NOS 611-631, continued from section above)
WmCas7x3 ---RVPADVRRALQLLGNP----GAVAAPVHYPQVKDAQIE--EKH---YLWFVANDD--
--------------EKTAGGNRH---------------LPRL-----HANSPGLPTLPRLV-----KREKDHSSNTGKPRRK--------
--------------------------------------------------
SER16298.1 ---GTDADIKHALQLLGDP----QYIGVPVHYPIAGNSTLE--NKH---FEWFGNNDK------
----ASVLRQKAQANSKNHHYQPKQQATPEEPQYLHTI-----
TKDSKQISLLKKNKIEDIENRDQQKHRYSNHRR---------------------------------------------------------
---
GwCas7x3 ---KVSDDVKRALELLGNP----DEITVPVHYPQLHNGLME--SKQ---FDWFVQNDK-----
----------SGRDQPANNRQH-------------LSSF-----TKDTEKLEPLIRIM-----------------RR-------------
-------------------------------------------------
PID64649.1 ---TMDADIQRAIELLGDP----GNVTSPVHYPQLDRKNIE--TKN---YEWFKQNEI--------
-------------EQQV---------------LKPI-----TKNTTHLTPFAR-------------------WEQG--------------
--------------------------------------------
WP_031436019.1 ---TMNADIYRTLELLGNP----QNVKRPVHYPQVENHNIE--QEN---YKWFVANDQ-
---------GSGKGRNKIDPAEKA---------------LKIL-----TENSDCLPTLSRLD------------------WRDE------
---------------------------------------------------
HEB50754.1 ---TMDETVFRALDLLGNP----AHVQRPVHYPQVREMDIE--DQT---FLWFVDNDK----
----------------QWKDA---------------LQPL-----TSSSTQLPPLTRRN-----------------KR------------
------------------------------------------------
HreCas7-11 --ANERPYIKQLRELLWVP---TGDNLPHVRYPELESKTKD-----VPGYTSLLKE--------
--KDL---------ADR---------------VSLL-----KA---PWKPWKPWS-----
GTAPHPDKGTNRLRASIVERDRIQRKTDTAKPEKKEETKVGKSSSSDIEKRYVGTVKWENDKKGYG
FI-LYGTD
CmaCas7-11 -DLSEYLHIKQLREILWFS----DKPVGTIEYPKLENKTNS----
RIPSYTDFVQEKDHETGFKNPKYQNL----------KSR---------------LHIL-----QN---PWNAWWKNE-----E----
HvmCas7-11 -DPMNFDHIRQLRELLWFL---PENVSANVRYPMLEKEDDG-----
TPGYTDFIKQEEPSTGKRNPSYLSS---------EKR---------------RNIL-----QT---PWKHWYLIP-----
PFQASAQSETVFE----------------------------------------GTVKWFDDKKGFGFIKINDGG
SmCas7x3 GDWHQRPHISGLRRLLTRY------PEIKARYPKLSQGEDK-----
EPGYIELKSQKDENGVPI----YNP----------NRE---------------LRVS-----EN---GPLPWFLLA-----KK-------
------------------------------------------------------------------
oral_meta -----VEERRKLLRLVMTP-----YKGLTACYPGLERENGR------PGYTDLKML------------
ATY----------DPY---------------RELVVQIGSNQ---PLRPWYEPG-----
KSFKPSPGNDCTGRGGSVSKSLISEPKVV-----------------PAIAPFCEGVVKWFNSVKGFGFI-ETKEQ
DisCas7-11 ----ELDFIENLKKLLWFP---EGDQAPRVCYPMLRKKDDP--NGN-SGYEELKDGE-------
----FKK---------EDR---------------QKKL-----TT---PWTPWA------------------------------------
----------------------------------------------
DsbaCas7-11 ---------NDLRKLLYLP---GEKQNPHVHYPTLKKEKKG----DPPGYEDLKKSFR----------
EKK---------LNR---------------RKML-----TT---LWEPWHK---------------------------------------
------------------------------------------
SstCas7-11 -DWEAAEHIKNLKKLLFLP---GEEQNPRVIYPALKQKDIP--NSRLPGYEELKKN--------
----LNM---------EKR---------------KEML-----TT---PWAPWHPIK-----K---------------------------
----------------------------------------------
CsbCas7-11
PQWHVIPHIDKLYKLLWVPFLNDSKLEPDVRYPVLNEESKGYIEGSDYTYKKLGDKDN----------LPY-
--------KTR---------------VKGL-----TT---PWSPWNPFQ-----
VIAEHEEQEVNVTGSRPSVTDKIERDGKMV--------------------------------------------
CjcCas7-11 -EWNGVKHIKDLRSLLSIP-----GDKPTVKYPTLNKDAEG--AISDYTYERLSDTKL-----
-----LPH---------DKR---------------VEYL-----RT---PWSPWNAFV-----KEAEYSPSEKSDEKGRETIRTKPK-
--------------------SLPSVKSIGKVKWFDEGKGFGIL-IMDDG
CbfCas7-11 -EWDRLEHIQGLRSLQRLL-------------PL-----------------------------------------------
--------------------------------------------------------------------------------------------
---------------------------
HvsCas7-11 --NEINEHMRNLLLFMTYY-----QNLPKVKYPDF------------DGYAKWRCS------------
YVE---------QDK---------------VEYF-----QN---RWIVAS------------------------------------------
----------------------------------------
HsmCas7-11 -TNFKI-YLNNFLQLLYWP----NNKKVKVRYPYLRQEDDP--EKL-PGYVELKKH------
------QML---------KDD---------------NRYSLFARPRA---VWLKWTEMV-----QRDKS-----------------------
---------------------------------------------
FmCas7-11 -SWYNIHTVNNLRTALYYV----EDDKIQVNYPKLKKDNES---DNRPGYVEMKKTA--
---------FPV---------RDI---------------L--------TT---PWWPWWPPT-----PPPMNQSGNQSYARSEEPARITE--
--------------------SQPEVYKTGTVKFYKHDKKFGFITMDGRE
DpbaCas7-11 -QWNKVEQVKNIRKLLQLSWNKGNAVEPEVRYPALREKDDE--NKR-
PGYVELKDNG-----------Y------------DA---------------GKKL-----VS---PWAPWHPIK-----K-------------
-------------------------------------------------------------
Domains
.........................................................................
.........................................................................
........................................................
SECTION 15 (SEQ ID NOS 611-631, continued from section above)
WmCas7x3 ---------------------------------------------
SER16298.1 ---------------------------------------------
GwCas7x3 ---------------------------------------------
WP_031436019.1 ---------------------------------------------
PID64649.1 ---------------------------------------------
HEB50754.1 ---------------------------------------------
HreCas7-11 EEIFVHRSGVADNSIPKEGQKVGFRIERGARGSHAVEVKAIE---
CmaCas7-11 ---------------------------------------------
HvmCas7-11 KDVFVHHSSIVGTGFKSLNEGDSVAFKMGVGPKGPCAEKVKKIGN
SmCas7x3 ---------------------------------------------
oral_meta RDIFVHFSAIRGEGYKILEPGEKVRFEIGEGRKGPQAINVIRIR-
DisCas7-11 ---------------------------------------------
DsbaCas7-11
SstCas7-11 ---------------------------------------------
CsbCas7-11 KEVSISKNSIRGNILLKKGQKVTFHIVQGLIPKAEDIEIAK----
CjcCas7-11 ---------------------------------------------
CbfCas7-11 ---------------------------------------------
HvsCas7-11 ---------------------------------------------
HsmCas7-11 ---------------------------------------------
FmCas7-11 NIHFAGNQICRPET--SLQSGDKVKFIEGENYKGPTALKVERLKG
DpbaCas7-11 ---------------------------------------------
Domains .............................................

### EXAMPLES

While several experimental Examples are contemplated, these Examples are intended non-limiting.

### Example 1

### Cas7-11 Family

New Cas7-11 family members were mined from bacterial and metagenomic data downloaded from NCBI, JGI, and ENA repositories. CDS were extracted using MetaGeneMark (Besemer and Borodovsky 1999; Zhu et al. 2010), and used to build BLAST databases (Camacho et al. 2009). Existing Cas7-11 family members (Makarova et al. 2019) were used as seeds to find putative orthologs. In parallel CRISPR arrays were identified using both CRISPRCasFinder (Couvin et al. 2018) and PILER-CR (Edgar 2007), and putative orthologs were filtered for proximity to CRISPR arrays.

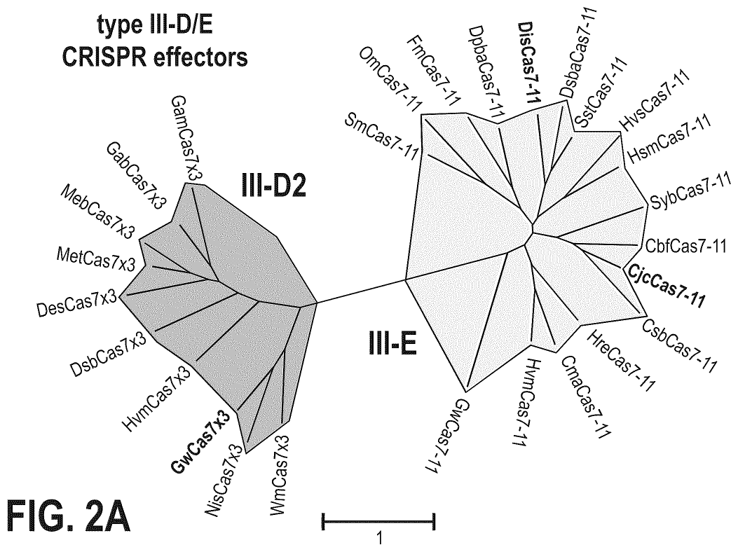

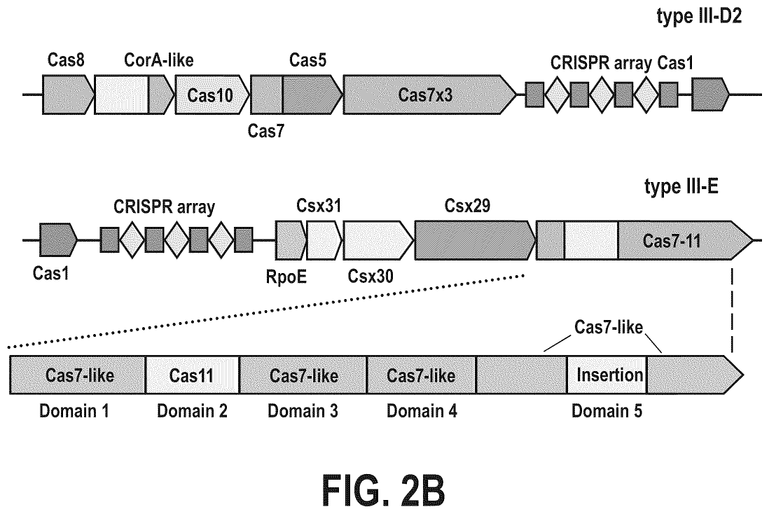

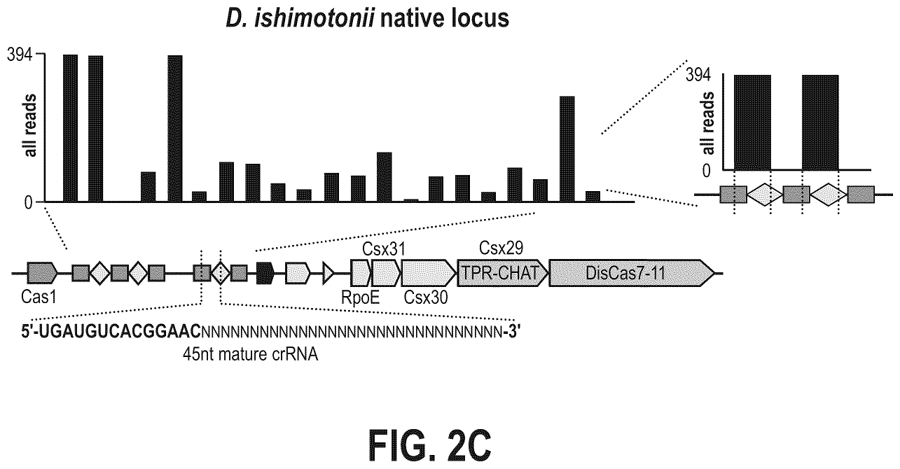

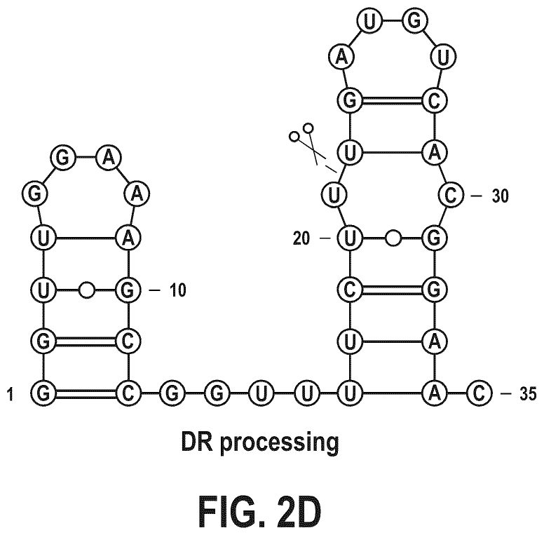

Cas7-11 family members were computationally identified and expressed. The Cas7-11 family is summarized in the trees presented in FIG. 2A. The type III-E Cas7-11 locus from Desulfonema ishimotonii (DisCas7-11) comprises multiple Cas7-like gRAMP domains, including a small subunit domain and an unknown insertion in the Csm3-like domain 5 (FIG. 2B). The CRISPR array was expressed in Desulfonema ishimotonii and processed into mature crRNAs containing 14-nt direct repeats (DRs) and ˜31 nt spacers (FIG. 2C). The cleavage site for processing and maturation is illustrated in FIG. 2D.

By screening a database of bacterial genomic and metagenomic sequences from diverse sources containing more than ˜11.6 billion protein sequences, the set of subtype III-E systems was expended to 17 loci from various bacteria and metagenomes (FIG. 2A). Apart from the identification of many additional type III-E loci, a novel subset of III-D systems (subtype III-D2) that was characterized by an the fusion of 3 Cas7 domains into a single protein, referred to as Cas7x3 was identified (FIG. 2A and FIG. 2B and Table 1). This similarity of the domain architectures, together with the phylogenetic analysis of the Cas7 domain (FIG. 2A), suggests an evolutionary path from subtype III D1 to III-D2 via the fusion of 3 Cas7 proteins, and then, from III-D2 to III-E via the fusion with an additional Cas7 protein and the Cas11 protein.

Examination of the extended set of type III-E systems confirmed that almost all III-E loci (15 of the 17) contained the TPR-CHAT protein (Csx29), and a majority encompassed uncharacterized proteins denoted Csx30 (10/17) and Csx31 (9/17) as well as the alternative sigma factor RpoE (8/17) (FIG. 1E). The frequent presence of these genes in the III-E loci suggests that the respective proteins play accessory roles in the CRISPR response. More than half of the III-E loci (9/17) also contain the adaptation gene Cas1 which, in 8 of these 9 loci, is fused to a reverse transcriptase (RT), in a common arrangement among the type III systems, as well as a CRISPR array (12/17). Searching the spacer sequences against the NCBI NT (nucleotide sequences) database did not identify any matches, in agreement with the previous observations that the majority of the CRISPR spacerome consists of “dark matter” that apparently reflects vast, still unexplored viromes. Furthermore, alignments of the Cas7 domains of the Cas7x3 and Cas7-11 proteins show conservation of acidic residues implicated in crRNA guided target cleavage (FIGS. 1F-1K). The activities of Cas7-11 and Cas7x3 proteins were experimentally characterized to assess the path of evolution that resulted in the unique Class 1 single protein effector that is predicted to function as a programmable RNase without collateral activity.

### Example 2

### Cas7-11 Defense

The RNA-guided RNA-targeting Cas7-11 was demonstrated to be capable of defense against ssRNA MS2 phage and RNA knockdown in bacteria.

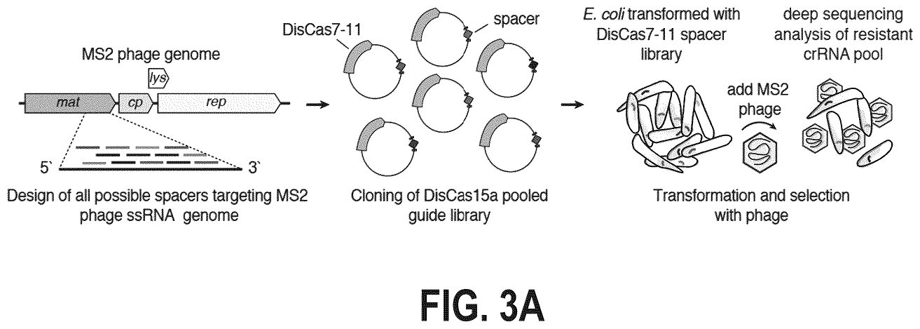

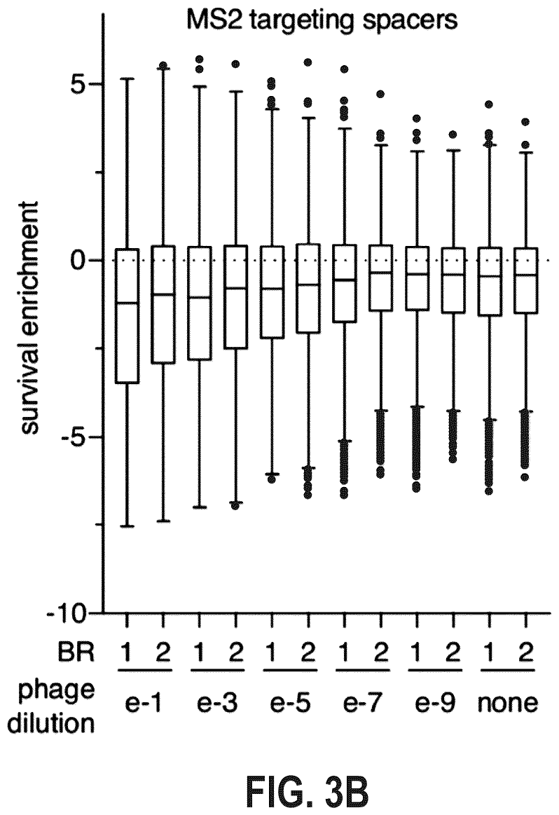

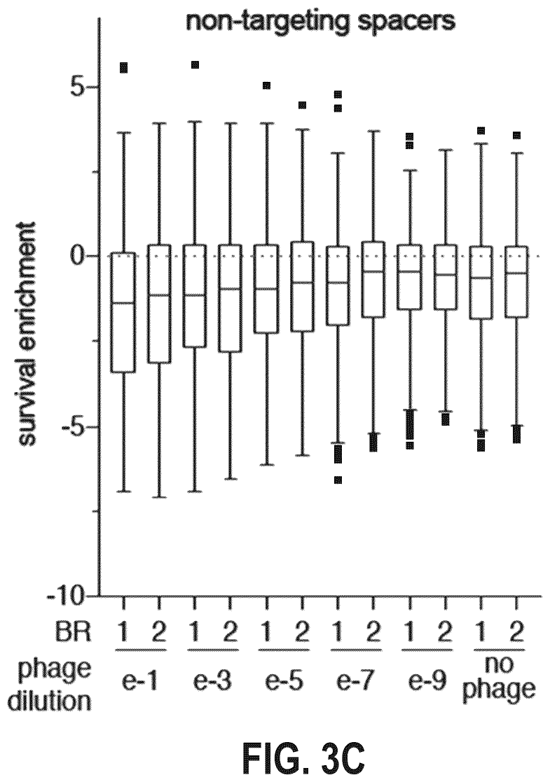

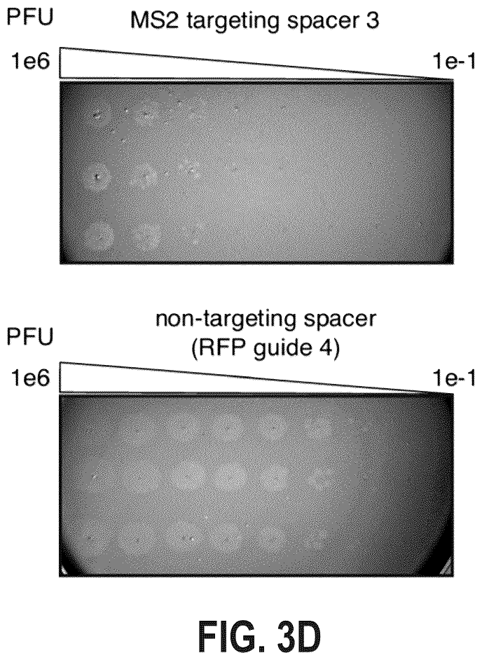

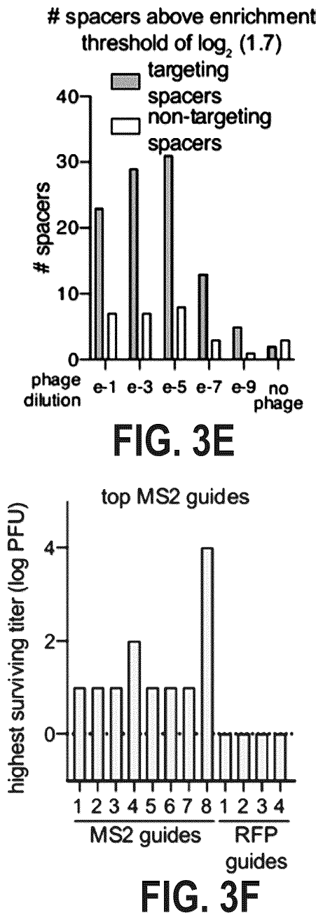

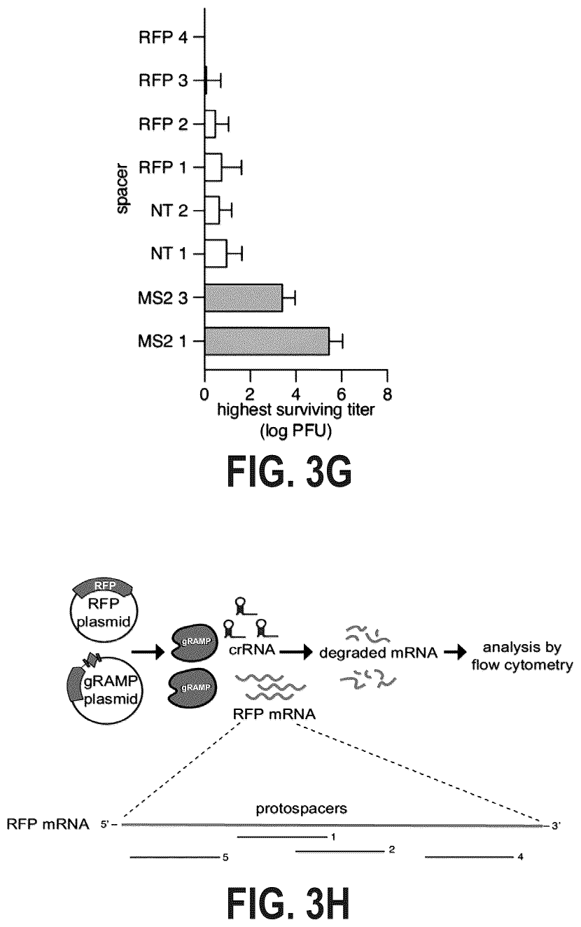

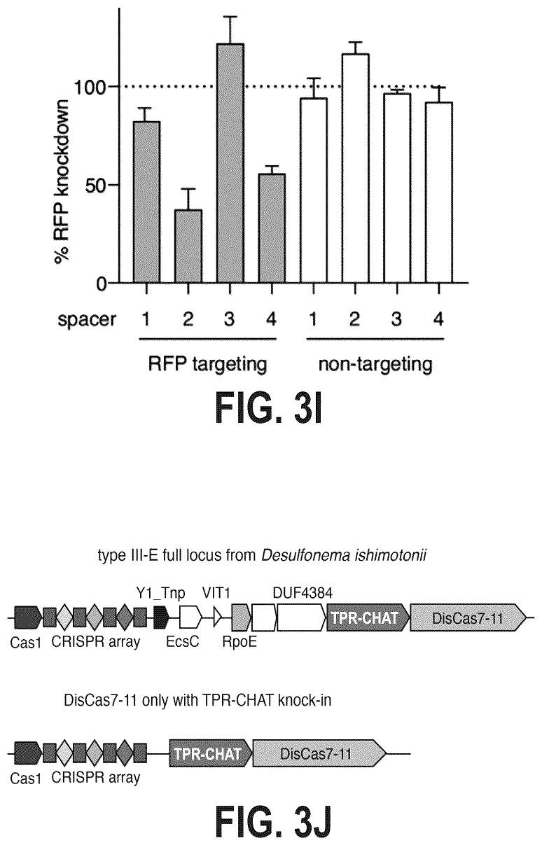

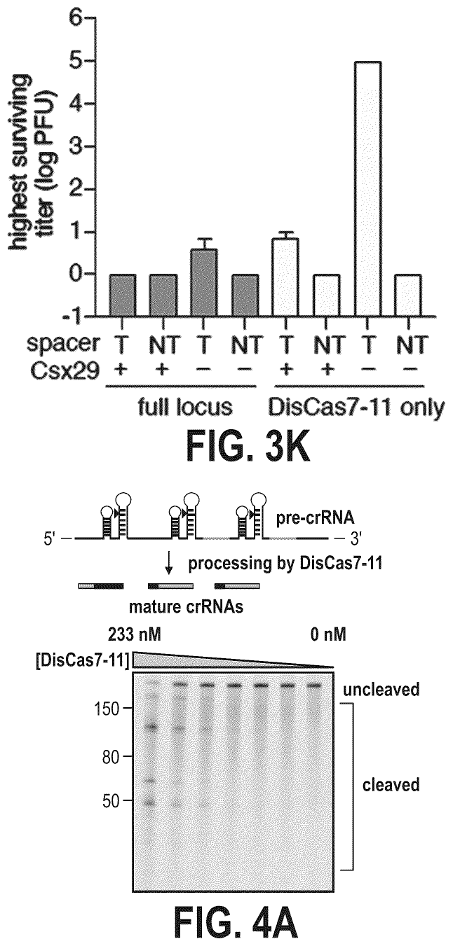

The CRISPR array screen of all crRNAs targeting the MS2 genome was performed (FIG. 3A). The results of the MS2 interference screen is shown in FIG. 3B (for MS2 targeting spacers) and FIG. 3C (for non-targeting spacers). Regarding FIG. 3B, the results of the MS2 interference screen are shown as box plots. Enrichment of DisCas7-11 spacers in the phage targeting condition denote survival of bacteria and enhanced representation of specific active spacers. Boxes denotes 25th and 75th percentiles with the median marked by the middle line. The whiskers are calculated via the Tukey method (1.5 times the inter-quartile range). Outliers are denoted by square symbols. FIG. 3C shows the results of the MS2 interference screen showing enrichment of DisCas7-11a non-targeting spacers across varying phage dilution amounts as box plots. Boxes denotes 25th and 75th percentiles with the median marked by the middle line. The whiskers are calculated via the Tukey method (1.5 times the inter-quartile range). Outliers are denoted by square symbols.

The enrichment of DisCas7-11 crRNAs in the phage targeting condition denotes the survival of bacteria and enhanced representation of specific active crRNAs. The results of the MS plaque dilution assay are shown in FIG. 3D. The assay shows significant survival of E. coli containing the top crRNA identified in the MS2 screen. In the non-targeting spacer, dilutions of phage up to 1e9 are capable of lysing bacteria. For the top targeting spacer, only dilutions up to 1e4 are capable of lysis because of DisCas7-11 interference.

The number of DisCas7-11a spacers that display survival enrichment over a threshold of 1.7 across different phage dilution conditions are shown in FIG. 3E. The quantification of resistance conferred by top MS2-targeting DisCas7-11 spacers compared against a panel of 4 non-targeting spacers is shown in FIG. 3F. The resistance is quantified as the highest surviving titer of MS2 phage that generates plaques in the dilution assay.

The quantification of resistance conferred by two MS2-targeting DisCas7-11 spacers compared against a panel of 6 non-targeting spacers is shown in FIG. 3G. Resistance is quantified as the final dilution of MS2 phage that generates plaques in the dilution assay.

The RFP knockdown assay containing a DisCas7-11 and spacers expression vector co-transformed with an RFP-expressing vector is illustrated in FIG. 3H, and the results from the knockdown assay are shown in FIG. 3I. The RFP knockdown assay by multiple targeting spacers is normalized to a non-targeting condition as determined by flow cytometry. DisCas7-11-mediate RFP knockdown was found to exceed 50%.

The DisCas7-11a full locus with TPR-CHAT nearby and DisCas7-11a effector expressed alone with TPR-CHAT are illustrated in FIG. 3H, and the MS2 interference activity of DisCas7-11a in the full locus and as a single effector with and without TPR-CHAT expression is shown in FIG. 3I.

### Example 3

### Cas7-11 Cleavage Activity

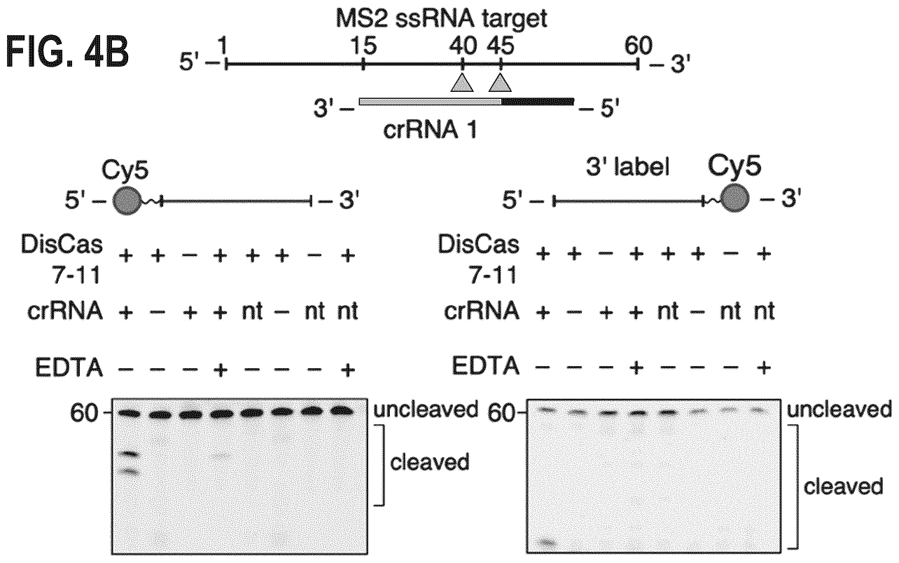

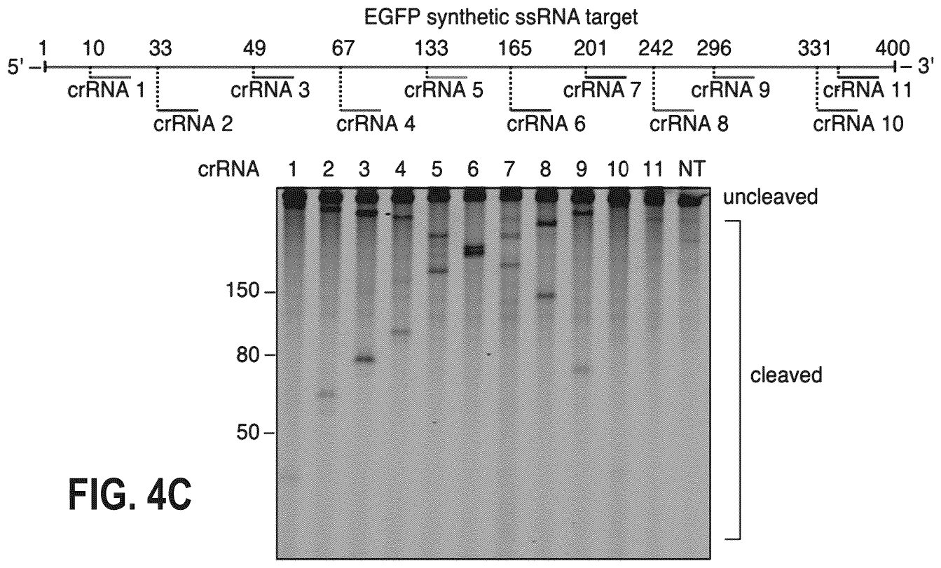

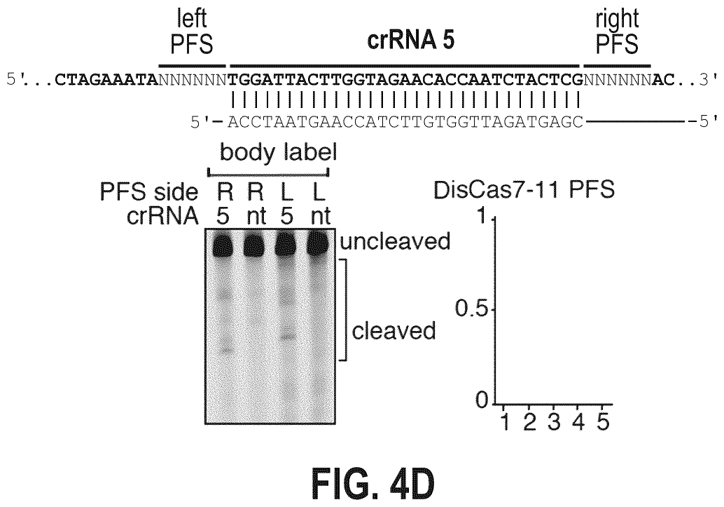

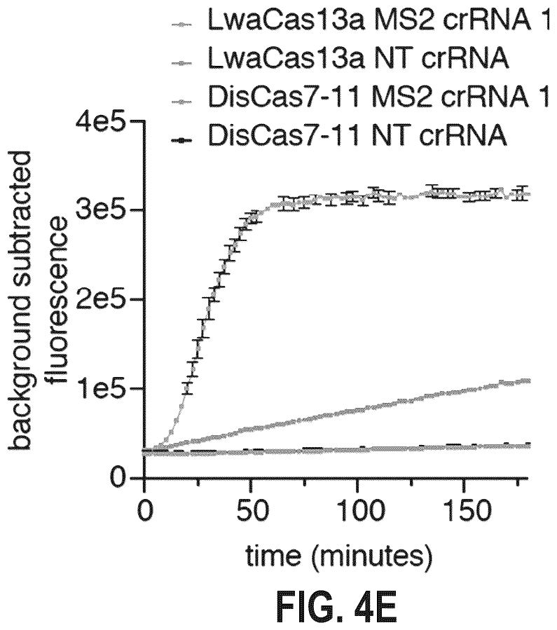

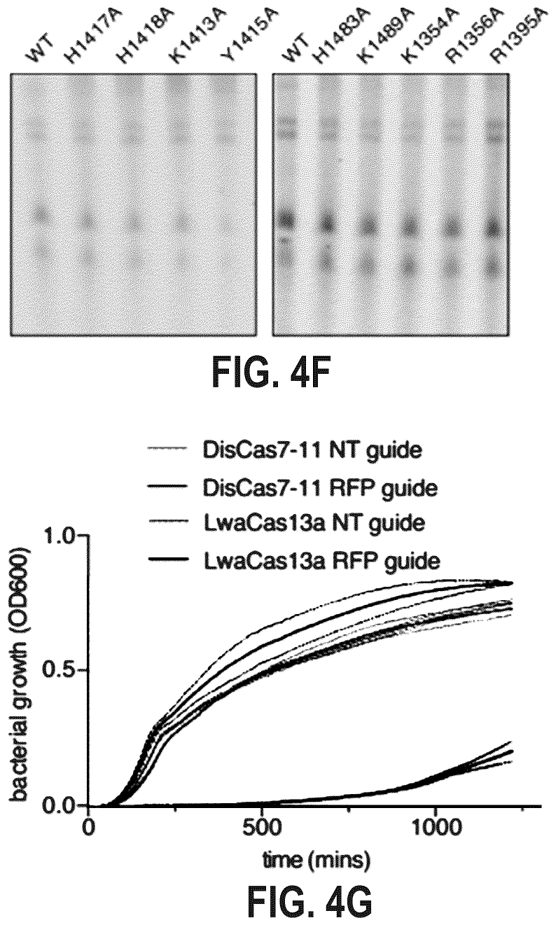

The DisCas7-11 programmable RNA cleavage activity was biochemically characterized. The CRISPR array processing assay for DisCas7-11 is illustrated in FIG. 4A and shows robust processing activity by DisCas7-11. The DisCas7-11 was assayed at concentrations ranging between 0 nM and 233 nM. The in vitori cleavage of ssRNA 1 target is illustrated in FIG. 4B and demonstrates that programmable DisCas7-11 RNA targeting is Mg+2 and crRNA dependent. Note that the targets are either 5′ or 3′ fluorescently labeled. The results from the cleavage of EGFP ssRNA at multiple sites with tiled crRNAs are shown in FIG. 4C. A varying cleavage pattern based on target position is observed. The protospacer flanking site (PFS) sequence screen is illustrated in FIG. 4D and shows a varying cleavage pattern based on target position and a lack of sequence preference flanking the target site for DisCas7-11 cleavage. The kinetics of fluorescence due to RnaseAlert collateral cleavage by DisCas7-11 or LwaCas13a targeting and non-targeting crRNA against the MS2 RNA target are illustrated in FIG. 4E. The processing activity of a synthetic DisCas7-11 CRISPR array by DisCas7-11 protein with predicted catalytic processing mutants in the protein insert region with a characteristic KxYxH catalytic triad motif is illustrated in FIG. 4F. The measurement of bacterial growth (OD600) during target interference by DisCas7-11 and LwaCas13 is shown in FIG. 4G.

### Example 4

### Cas7-11 Catalytic Residues

The catalytic residues in DisCas7-11 were identified.

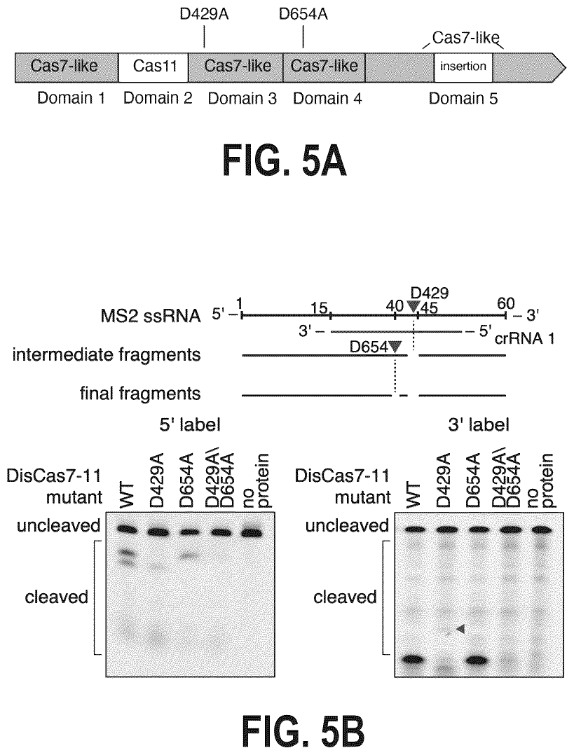

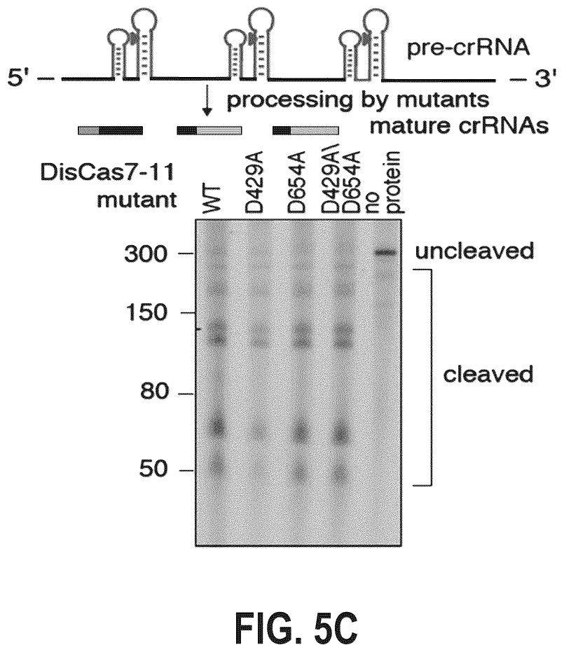

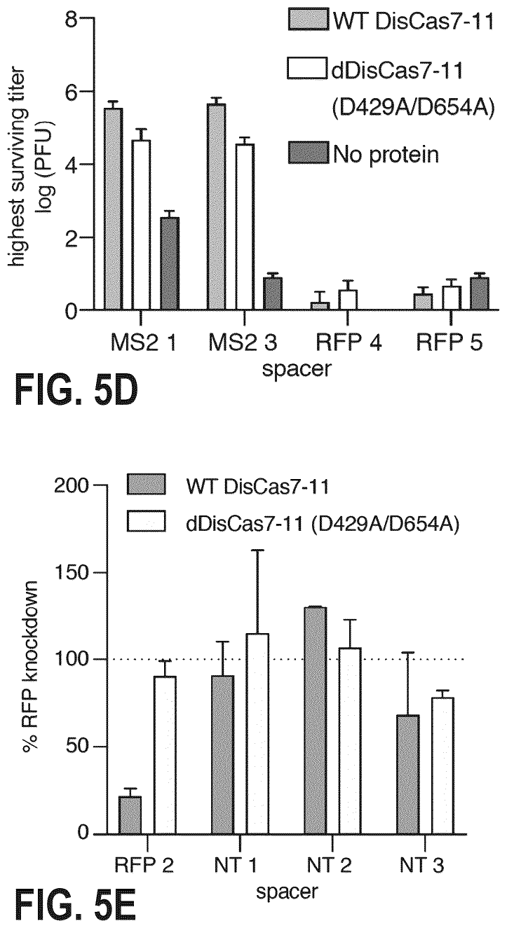

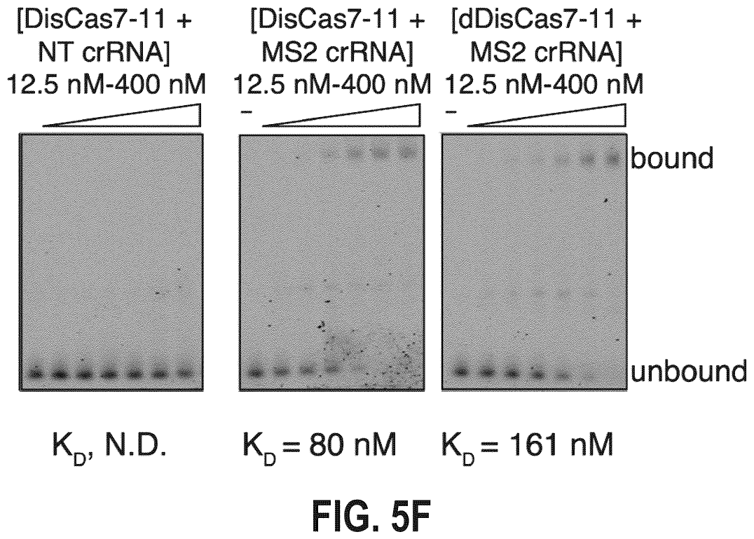

The protein domains of DisCas7-11 with putative catalytic residues highlighted in Cas7-like domains 3 and 4 are illustrated in FIG. 5A. The results from the cleavage of synthetic ssRNA target with two cRNAs and wild type, D429A, D654A, and D429A/D654A DisCas7-11 proteins are shown in FIG. 5B. The cleavage is affected by the single mutants and completely inactivated by the double mutant D429A/D654A. The processing of a synthetic DisCas7-11 CRISPR array by catalytic DisCas7-11 mutants is not affected by D429A and D654A catalytic mutations (FIG. 5C). The MS2 phage interference in bacteria is affected by D429A/D654A mutations, although there is remaining residual interference above the lack of DisCas7-11 altogether (FIG. 5D). The RFP knockdown in E. coli is prevented by the catalytic mutation D429A/D654A in DisCas7-11 (FIG. 5E).

The DisCas7-11:crRNA complex binding to a complementary MS2 ssRNA target is determined by electrophoretic mobility shift assay (EMSA) and illustrated in FIG. 5F. An EMSA is performed for both the WT DisCas7-11 and dead DisCas7-11 complexes and is compared to WT DisCas7-11 in complex with a non-targeting crRNA. The Kd value for the WT and dead DisCas7-11 complexes are shown below corresponding gels.

### Example 5

### Cas7-11 Cleavage Sites

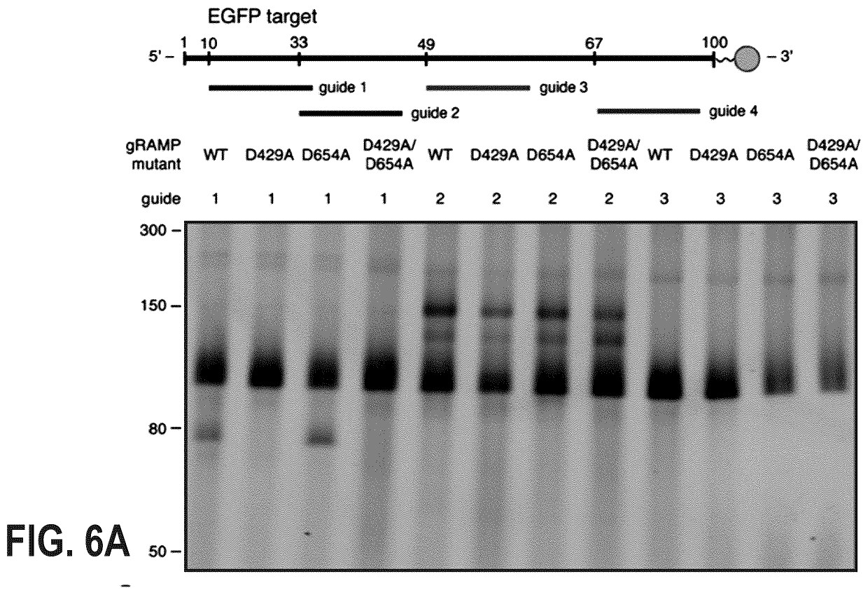

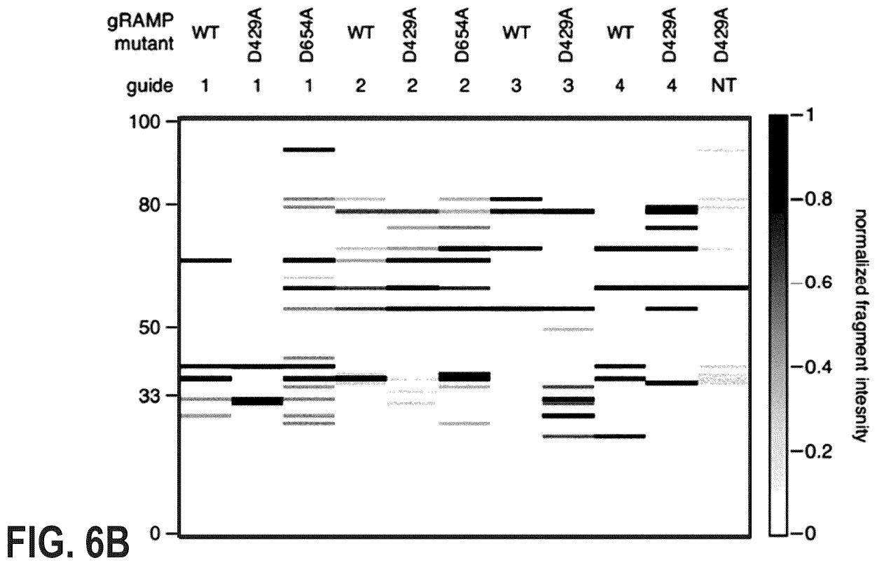

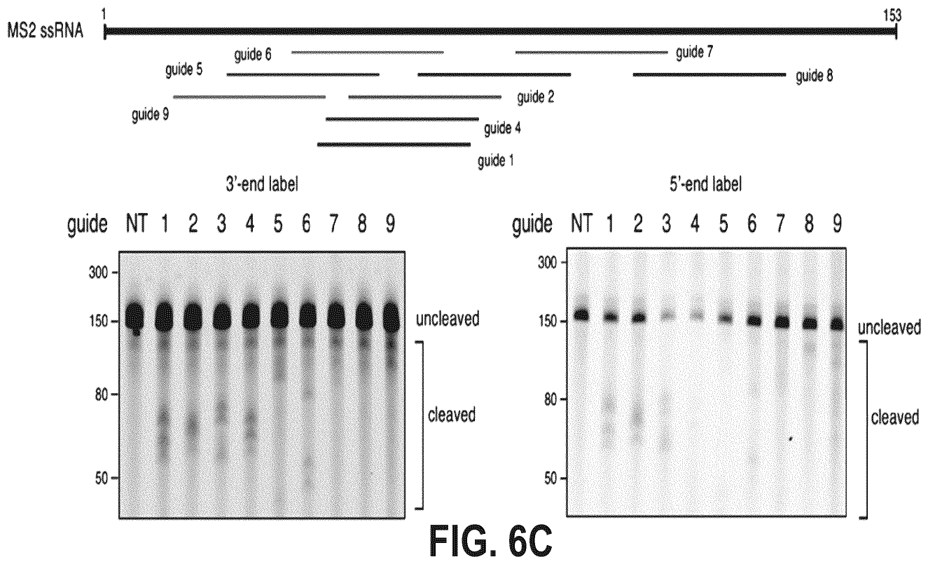

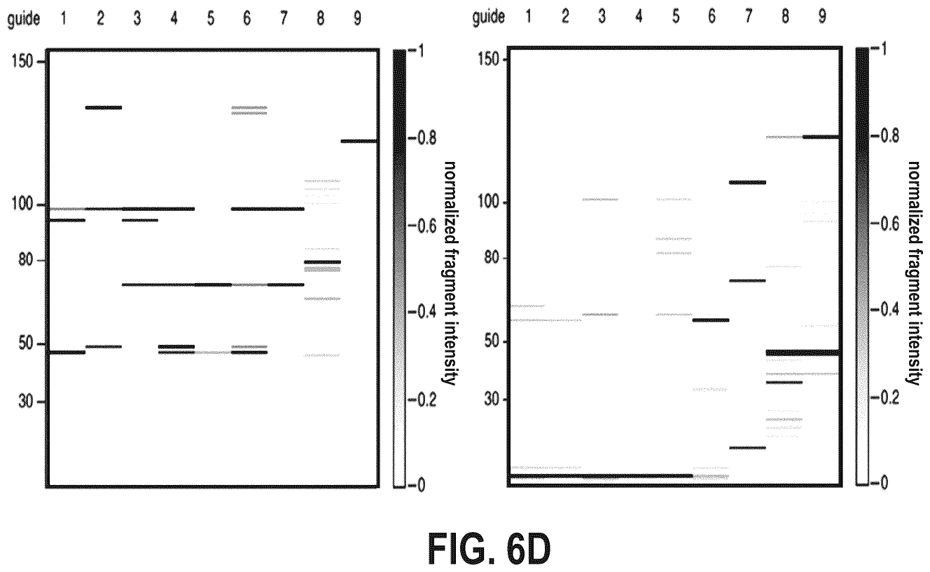

DisCas7-11 cleavage sites were characterized. The DisCas7-11 guides targeting multiple sites on a synthetic 100 nt EGFP ssRNA target are illustrated in FIG. 6A and the cleavage patterns of each EGFP-targeting guide incubated with DisCas7-11 on a 3′ labeled EGFP ssRNA are shown in FIG. 6B. The DisCas7-11 guides targeting of multiple sites on either MS2 ssRNA or ssRNA target 2, two targets of equivalent length (153 nt), are illustrated in in FIG. 6C. The guides are designed to target sites at the same position on both targets in order to better understand whether sequence specific cleavage preferences exist and whether cleavage cut sites are position dependent. The cleavage patterns of each guide incubated with MS2 ssRNA or ssRNA target 2 are shown in FIG. 6D.

### Example 6

### Cas7-11 Orthologs Activity

The CRISPR processing and RNA cleavage activity were demonstrated for different DisCas7-11 orthologs.

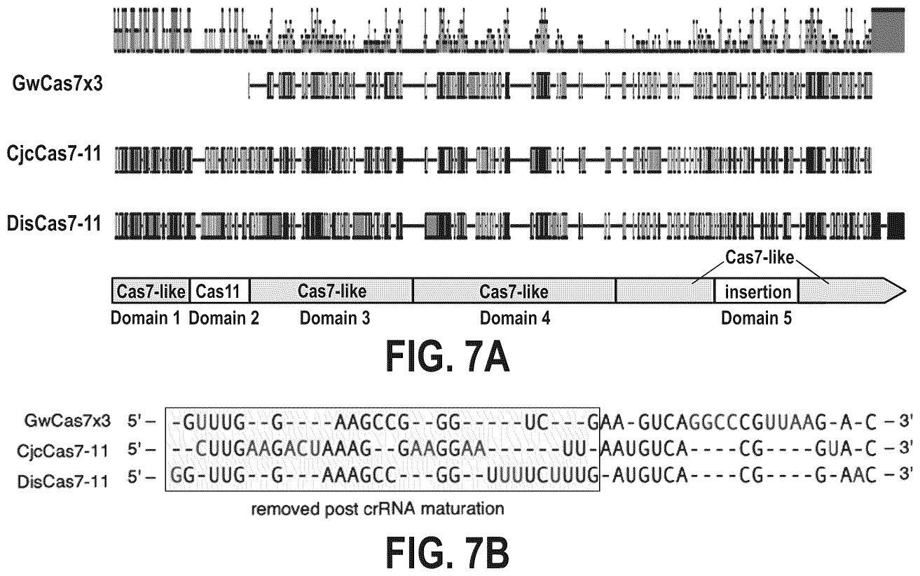

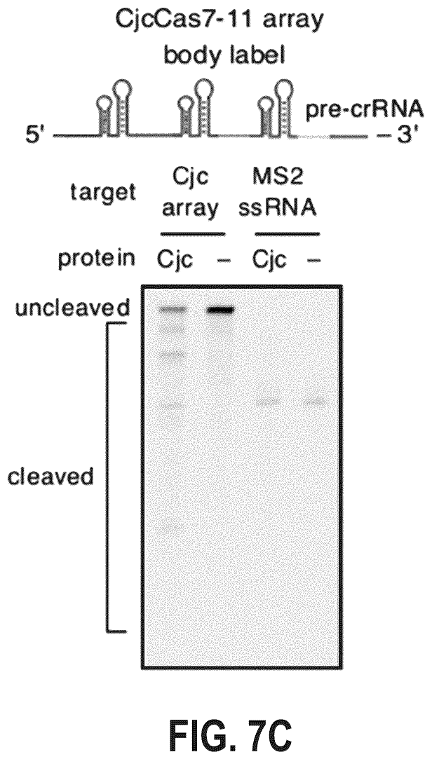

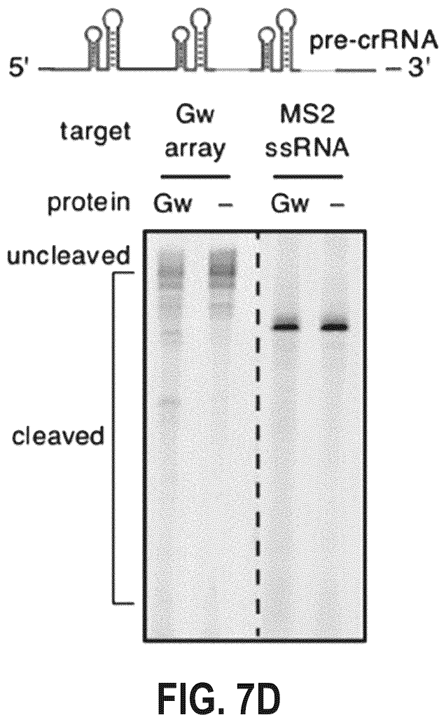

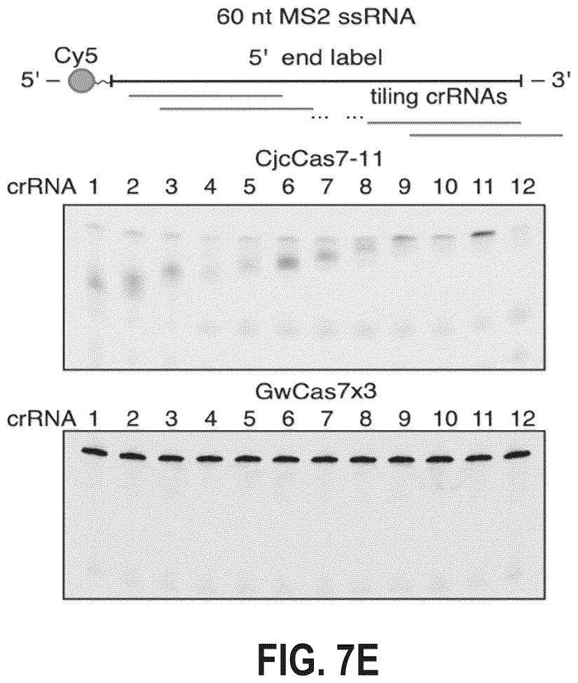

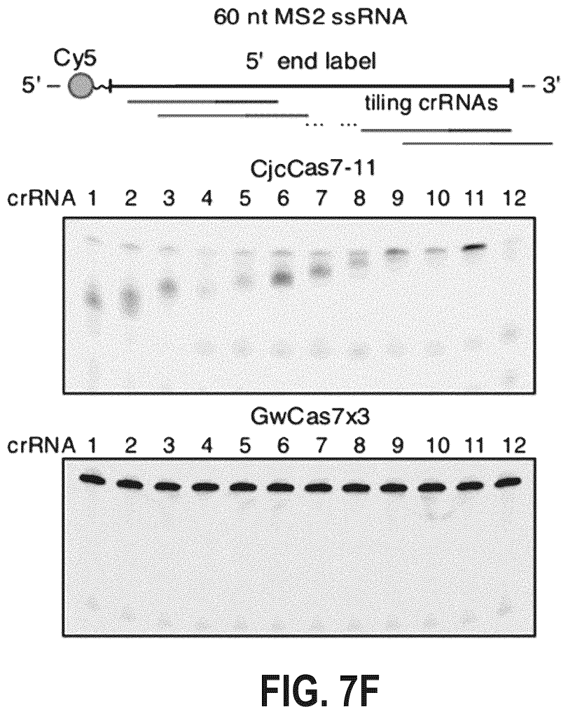

The schematics of three Cas7-11 orthologs (GwCas7-11, CjcCas7-11, and DisCas7-11) chosen for additional characterization are shown in FIG. 7A, wherein a multiple sequence alignment of the three orthologs compared against the domain organization of a typical Cas7-11 protein is shown. The multiple sequence alignments of the DR sequences for GwCas7-11, CjcCas7-11, and DisCas7-11 are shown in FIG. 7B. Mismatches against the consensus are highlighted in grey and the grey region shows the sections of the DRs that are cleaved from the pre-crRNAs to generate mature crRNAs.

The activity of the GwCas7-11 orthologs at concentrations between 2 uM to 125 nM was assessed. The activity of Cas7-11 from Candidatus Jettenia caeni (CjcCas7-11) shows robust processing of CRISPR arrays specific to the CjcCas7-11 locus (FIG. 7C). The activity of Cas7-11 from ground water metagenomes (GwCas7-11) shows robust processing of CRISPR arrays specific to the GwCas7-11 locus (FIG. 7D). MS2 ssRNA target cleavage by GwCas7-11 and CjcCas7-11 incubated with their cognate crRNAs using guides complementary to the MS2 ssRNA target is illustrated in FIG. 7E. The incubation of GwCas7x3 and CjcCas7-11 proteins with their cognate crRNAs complementary to the MS2 ssRNA target at varying positions along the target is illustrated in FIG. 7F.

### Example 7

### Cas7-11 RNA Knockdown in Mammalian Cells

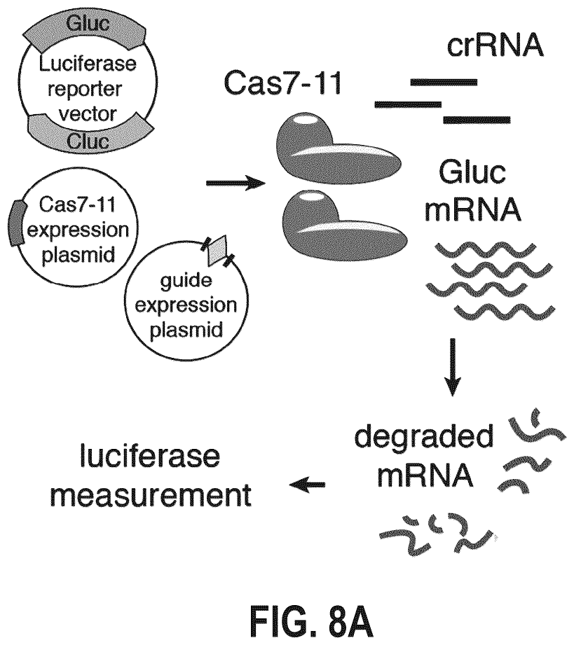

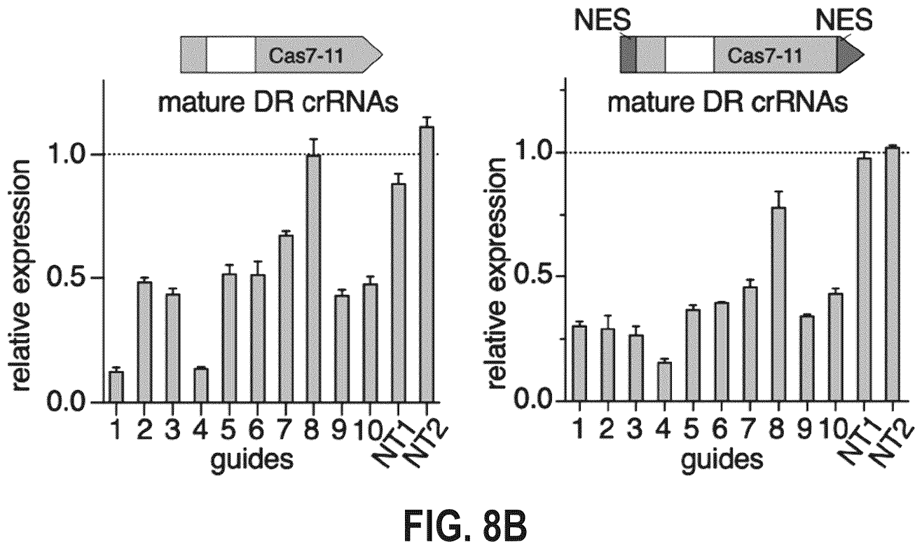

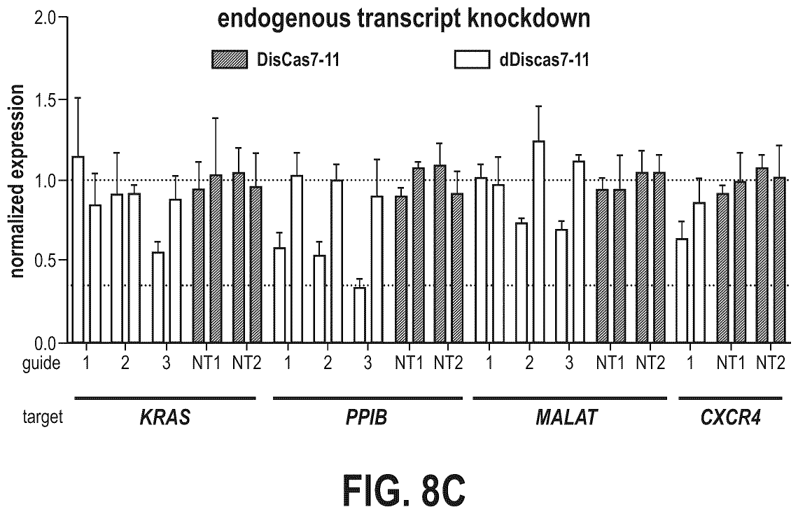

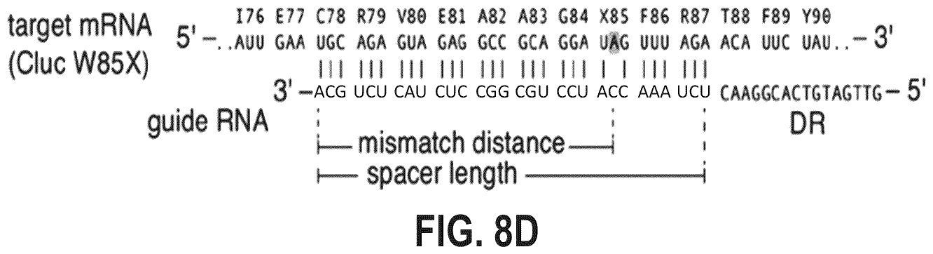

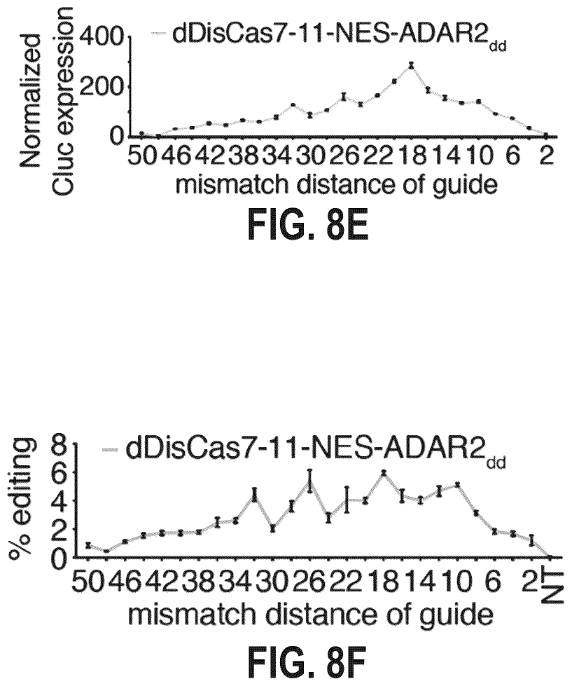

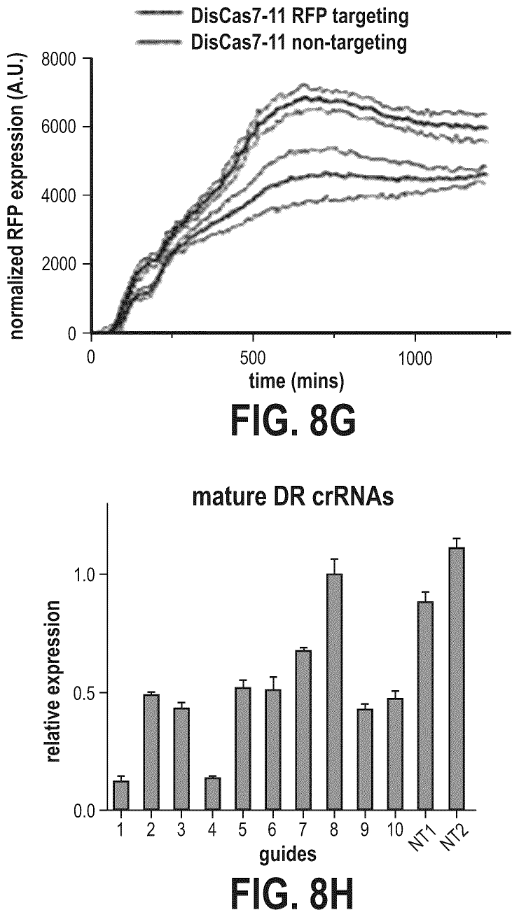

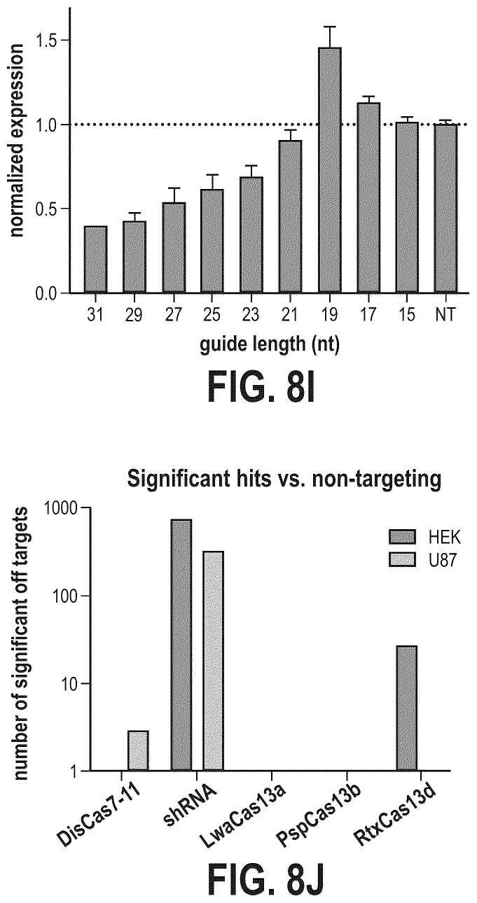

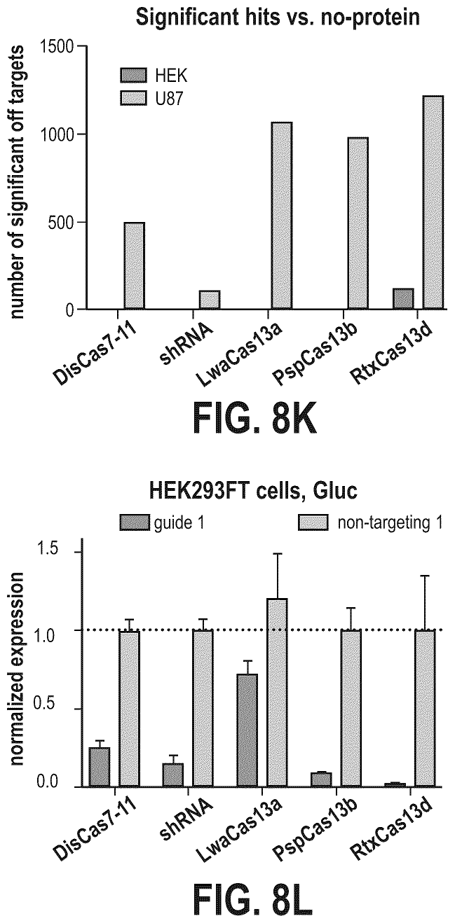

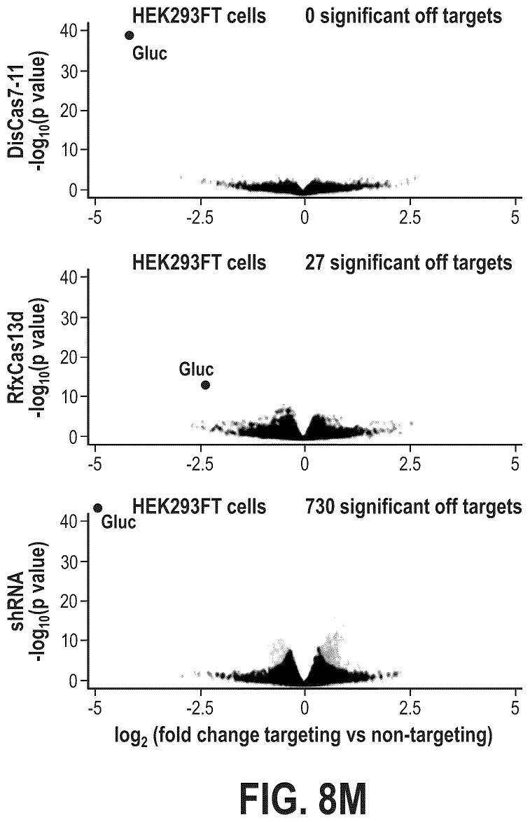

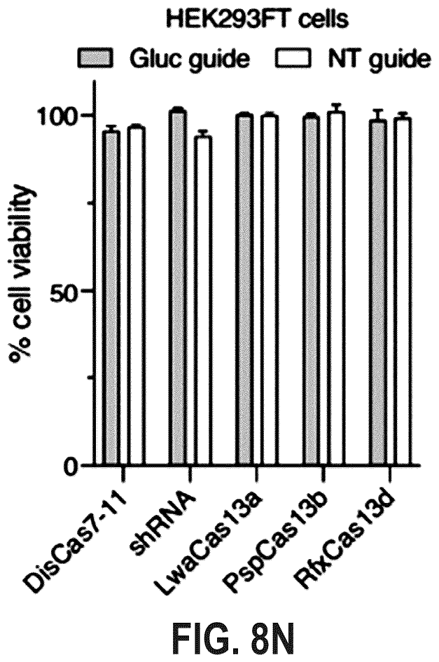

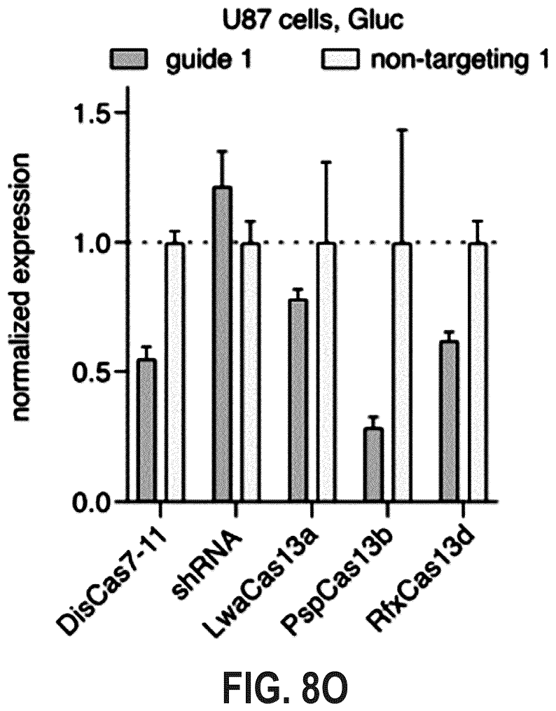

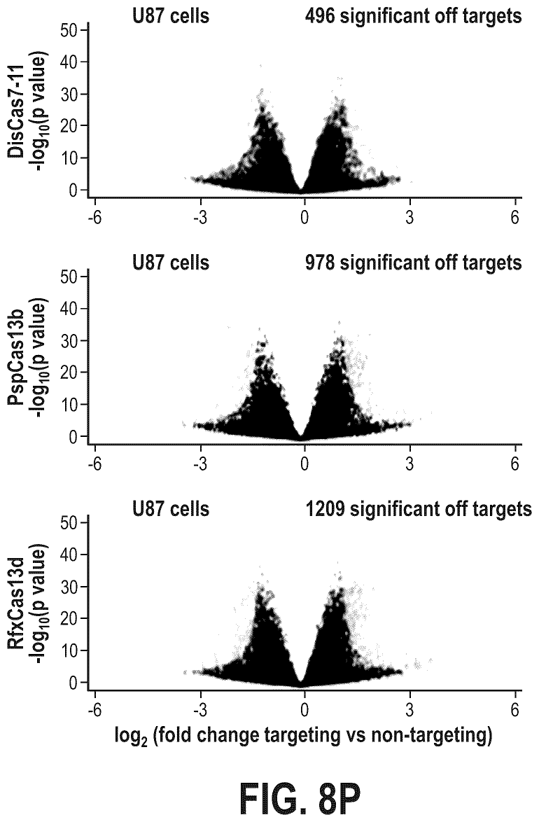

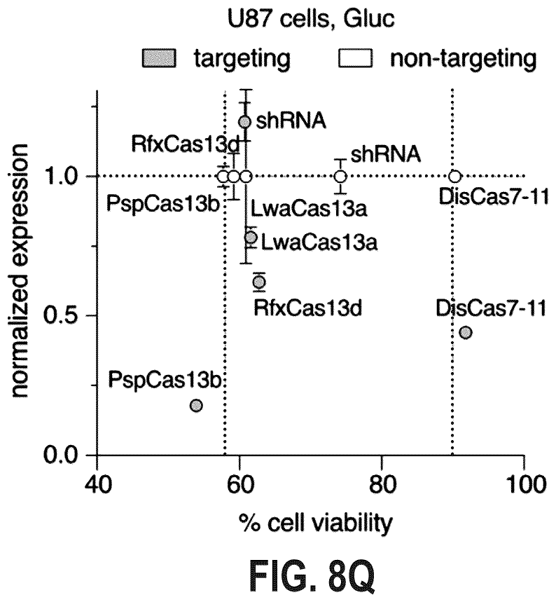

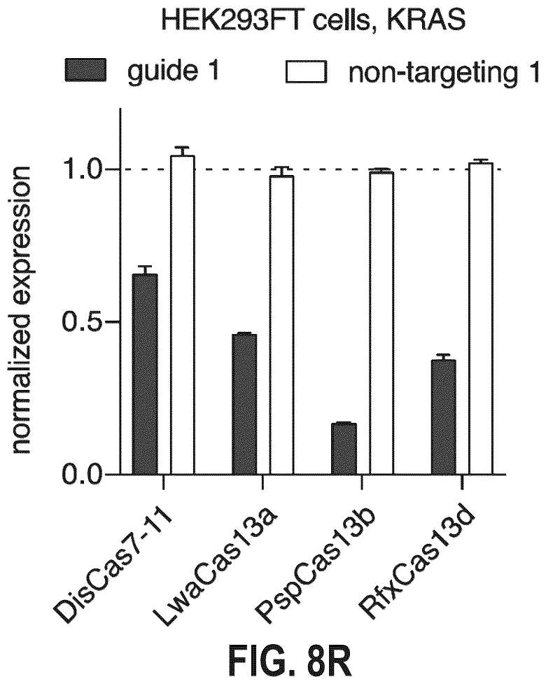

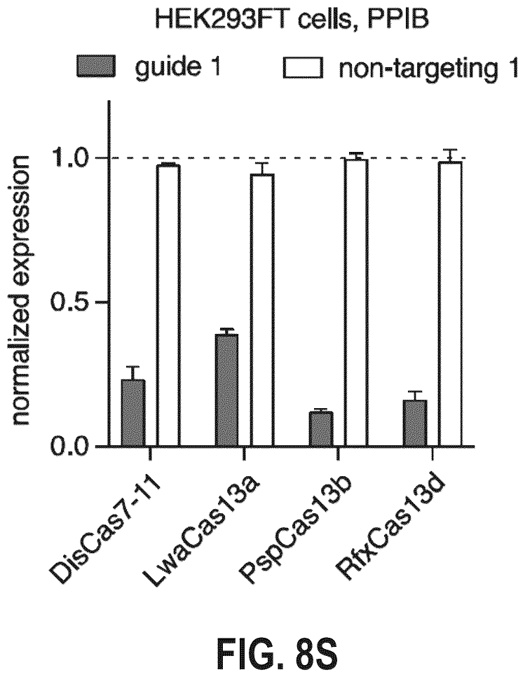

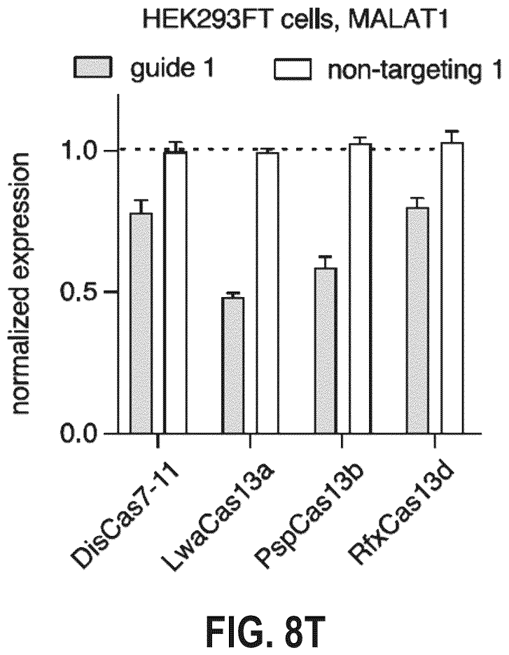

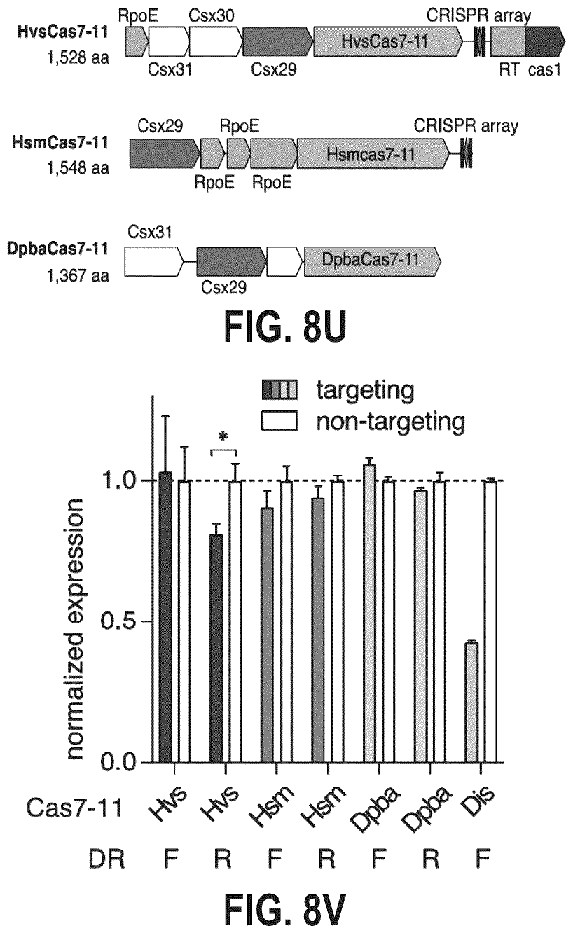

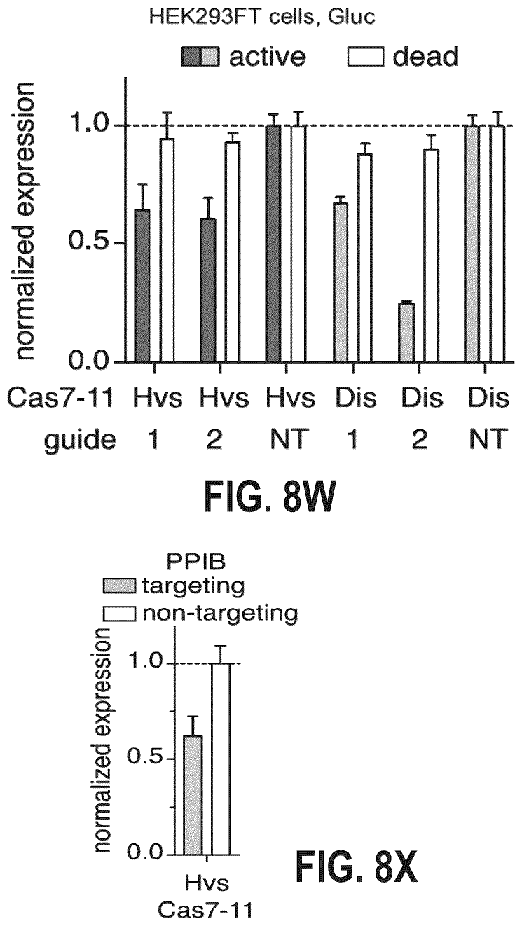

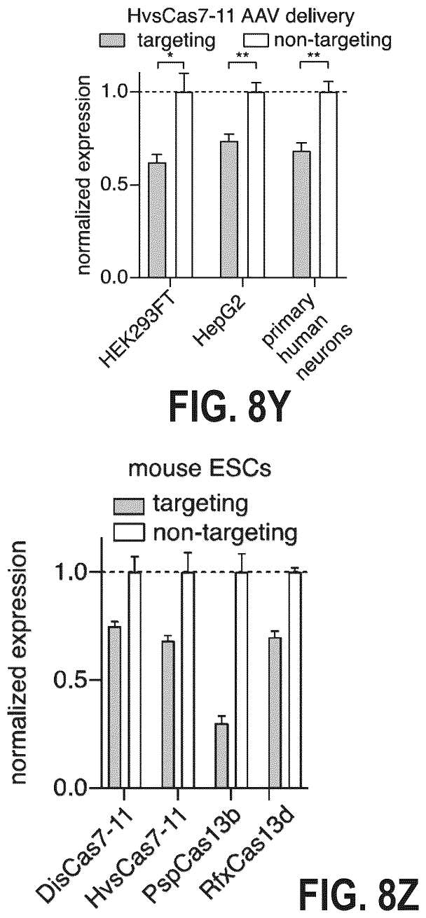

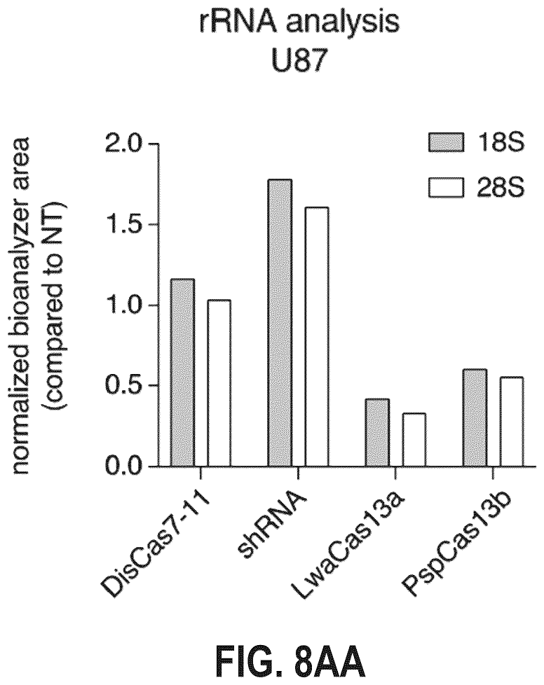

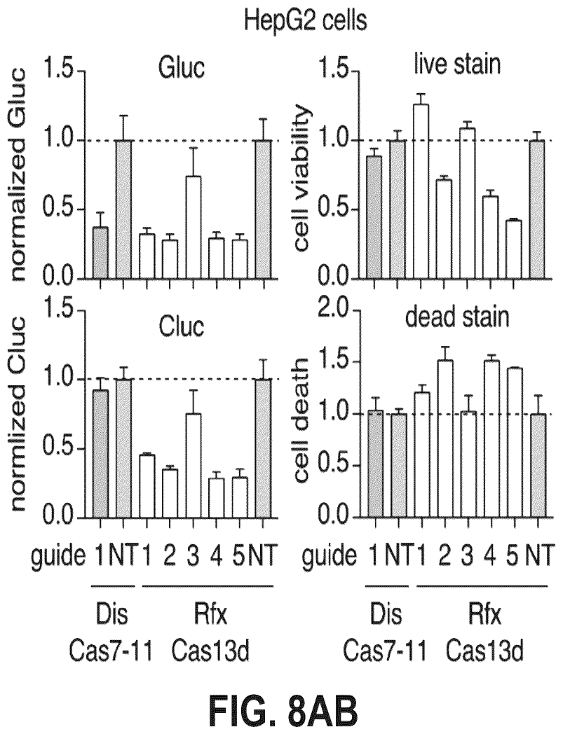

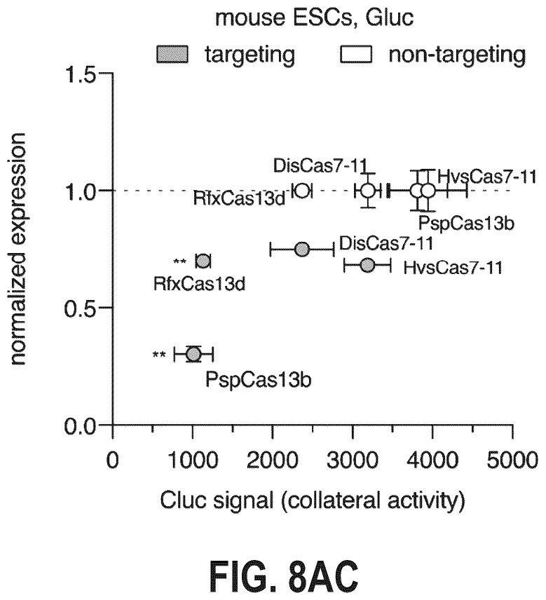

The knockdown of RNA in mammalian cells by DisCas7-11 was evaluated. The schematic of a luciferase reporter assay for RNA knockdown using a vector expressing human codon-optimized DisCas7-11 and guide RNA is shown in FIG. 8A. The knockdown activity of DisCas7-11 against the Gaussia luciferase (Gluc) transcript normalized to two non-targeting conditions is illustrated in FIG. 8B. Note that the dotted line in this figure represents 85% knockdown. The knockdown activity of DisCas7-11 against the multiple endogenous transcripts normalized to two non-targeting conditions is illustrated in FIG. 8C. Note that the dotted lines in this figure represent background expression and 65% knockdown by DisCas7-11.

RNA A-to-I editing of Cypridina luciferase (Cluc) mRNA W85X mutation in mammalian cells by dead DisCas7-11a-NES-ADAR2 was investigated. Guides were designed with mismatch distances between 2-50 nt. Editing was measured by two methods: 1) restoration of Cluc luciferase activity that is normalized to the non-targeting guide condition (FIG. 8E) and 2) amplicon sequencing of the targeted adenosine to measure percent conversion of the adenosine to inosine (FIG. 8F).

The RFP knockdown by DisCas7-11 or LwaCas13a in E. coli bacteria was assessed (FIG. 8G). The knockdown of Gluc mRNA in mammalian cells by DisCas7-11 with a panel of guides containing the mature DR sequence was assessed (FIG. 8H). The guides were designed to be tiled across the Gluc transcript. The knockdown of Gluc mRNA in mammalian cells by DisCas7-11 with a panel of guides of different lengths was assessed (FIG. 8I). Comparison of the number of significant off-targets when targeting the Gluc transcript in HEK293 FT and U87 cells by DisCas7-11, shRNA, LwaCas13a, PspCas13b, and RfxCas13d using transcriptome-wide RNA-sequencing data is shown in FIG. 8G. Off-targets were determined by significance testing of differentially expressed transcripts between targeting and non-targeting guide conditions. Comparison of the number of significant off-targets when targeting the Gluc transcript in HEK293 FT and U87 cells by DisCas7-11, shRNA, LwaCas13a, PspCas13b, and RfxCas13d using transcriptome-wide RNA1217 sequencing data is shown in FIG. 8K. Off-targets were determined by significance testing of differentially expressed transcripts between the targeting guide condition and cells only expressing EGFP. The data were mean (n=3)±s.e.m.

Given that DisCas7-11 lacked collateral activity in vitro or in vivo, the efficiency and effects of targeting with DisCas7-11, multiple Cas13 family members, and RNA interference through small hairpin RNA (shRNA) was compared. The activity in HEK293 FT cells was assessed, for which no detectable growth impact from Cas13a targeting has been reported. Comparing Gluc knockdown between DisCas7-11, LwaCas13a, PspCas13b, RfxCas13d, and shRNA, comparable knockdown efficiencies was found for the different systems, with the exception of LwaCas13a, which could only reach ˜35% knockdown (FIG. 8L). By assessing the specificities of the different systems by whole transcriptome profiling in HEK293 FT cells and comparing between targeting and non-targeting guide conditions, it was found that there were more differentially expressed genes for shRNA and RfxCas13d than for DisCas7-11 (FIG. 8M). LwaCas13a and PspCas13b had minimal off-target effects on the transcriptome when comparing targeting versus non-targeting guide conditions, and none of these knockdown tools had any effect on HEK293 FT cell viability (FIGS. 8M-8N). In addition, when comparing targeting conditions of the different tools to a no-protein, GFP condition, only RfxCas13d expression was found to result in detectable differential gene expression. Because cell toxicity of Cas13 has been reported in certain cell types, such as the U87 glioblastoma cell line7, the degree of toxicity due to the collateral activity of Cas13 may be cell-type specific. Thus, the comparison of shRNA, Cas13a/b/d, and Cas7-11 knockdown activities was repeated in U87 cells, finding efficient knockdown by DisCas7-11, PspCas13b, and RfxCas13d, less efficient knockdown with LwaCas13a, and no activity from shRNA (FIG. 8O). Whereas DisCas7-11 and the Cas13 variants showed minimal differential gene expression off-targets when comparing between targeting and non-targeting conditions, evaluation of targeting conditions versus the no protein, GFP-only control showed increased numbers of differentially expressed genes in RfxCas13d, LwaCas13a, and PspCas13b compared to DisCas7-11 (FIG. 8P). Moreover, Cas13 enzymes and shRNA caused cell toxicity resulting in ˜30-50% cell death, whereas DisCas7-11 expression had minimal impact on cell viability (FIG. 8Q), consistent with the specific knockdown demonstrated by transcriptome sequencing. The observation that certain cell lines can be more susceptible to non-specific RNA degradation than others is consistent with other reports on substantial RNAi toxicity in diverse cell lines. Moreover, the ineffectiveness of the RNAi constructs in U87 cells herein, in contrast to their functionality in HEK293 FT cells, is consistent with the low Dicer and Argonaute 2 expression in U87 cells, causing poor on-target knockdown, with off-target induced toxicity. The highly specific and low toxicity knockdown activity of DisCas7-11 compared to other RNA knockdown tools, RNAi and Cas13, could make Cas7-11 proteins more generally applicable and appropriate tools for RNA knockdown across diverse cells and tissues.

### Example 8

### Heterologous Expressions of Type-III-E CRISPR Cas System

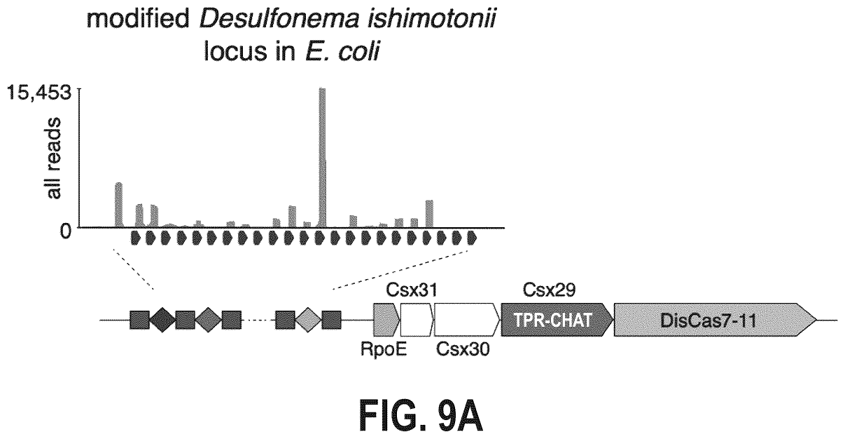

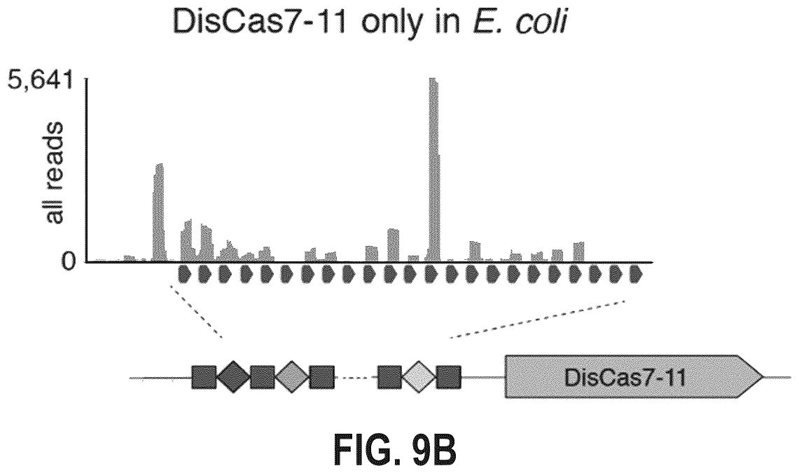

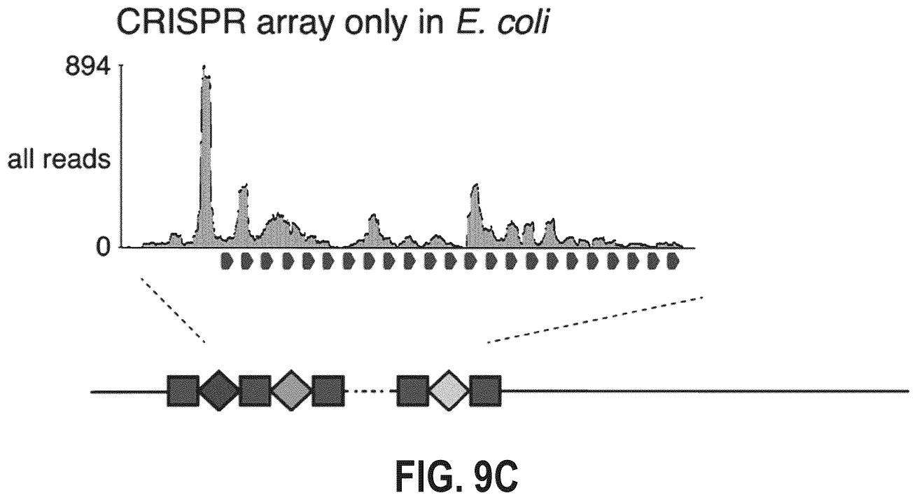

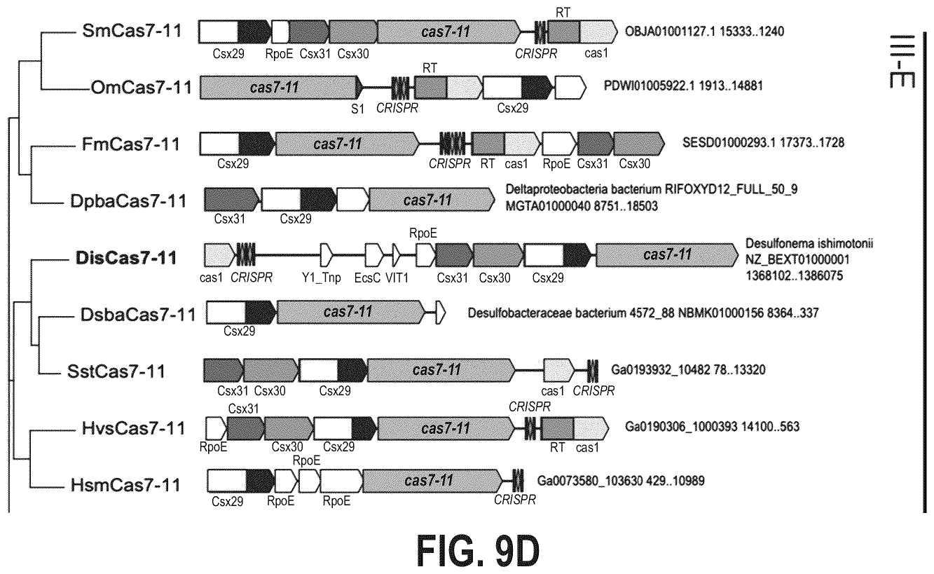

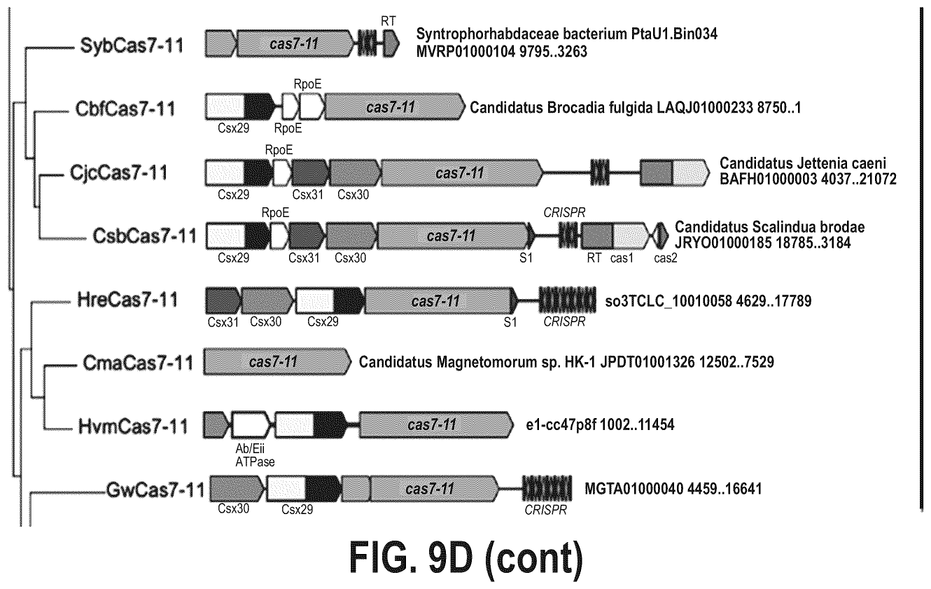

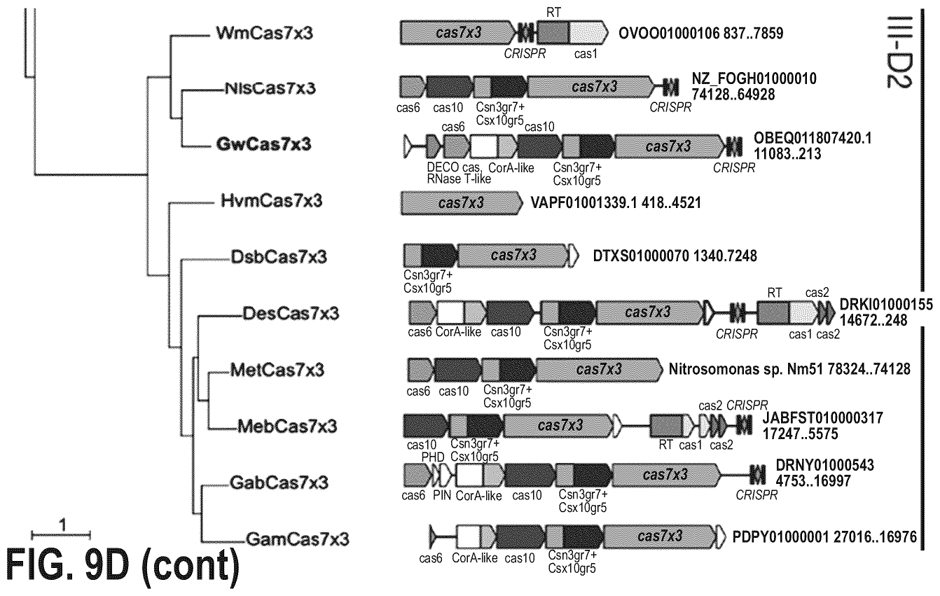

The heterologous expression of the Desulfonema ishimotonii Type III-E CRISPR-Cas system and associated CRISPR array were performed. The Desulfonema ishimotonii Type III-E full locus matures crRNAs (FIG. 9A), single effector protein DisCas7-11 matures crRNAs in E. coli. (FIG. 9B) and DisCas7-11 CRISPR array without any effector or accessory proteins (FIG. 9C) were expressed. Additional type III-E family members and loci architecture are illustrated in FIG. 9D.

### Example 9

### Cas7-11 Dilution Assays

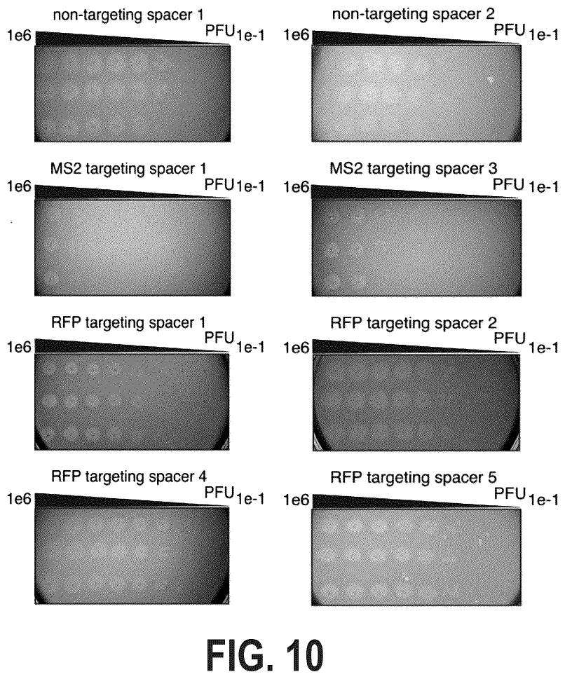

Dilution assays with DisCas7-11 and different spacers were performed. The DisCas7-11 assays for MS2 targeting spacers, non-targeting spacers and RFP targeting spacers were performed with dilution of phage up to 1e-1 (FIG. 10). The assay shows that bacteria with DisCas7-11a and MS2 targeting guides can survive at higher viral loads

### Example 10

### Effects Genes on MS2 Interference

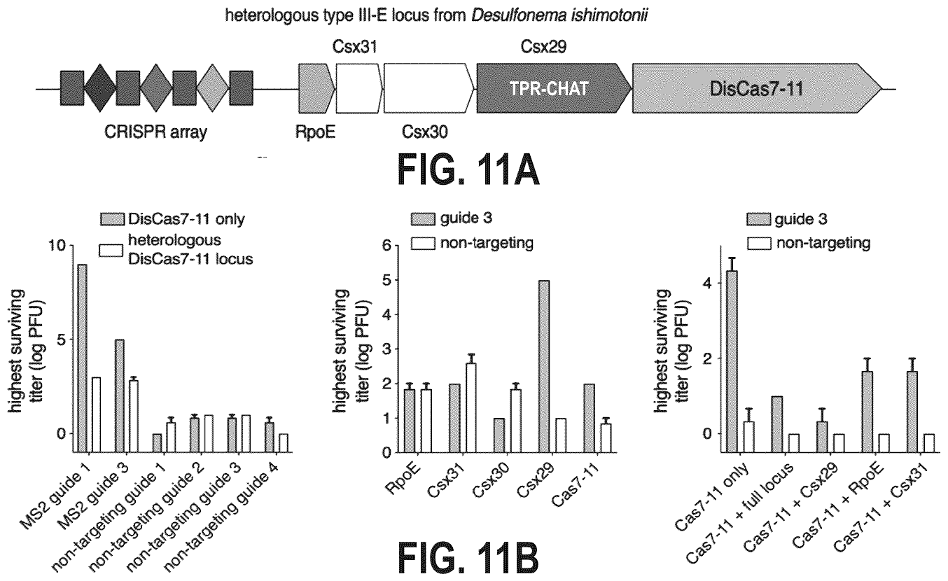

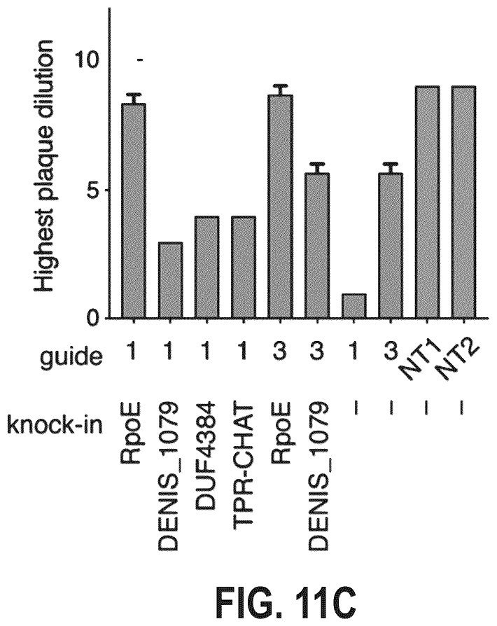

The effects of genes in the Desulfonema ishimotonii type III-E CRISPR-Cas locus on MS2 interference were analyzed. The Type III-E locus of the Desulfonema ishimotonii is illustrated in FIG. 11A. Results of the phage plaque assay of the incomplete Type III-E DisCas7-11 CRISPR-Cas locus are shown in FIG. 11B. The knock-out of TPR-CHAT shows the highest positive effect on the interference by DisCas7-11. Results from the phage plaque assay of the individual gene knock-in together with of the Type III-E DisCas7-11 CRISPR-Cas system are shown in FIG. 11C. Knock-in data shows that RpoE has an inhibitory effect on DisCas7-11.

### Example 11

### Cas7-11 Pre-crRNA Processing to DR Mutations

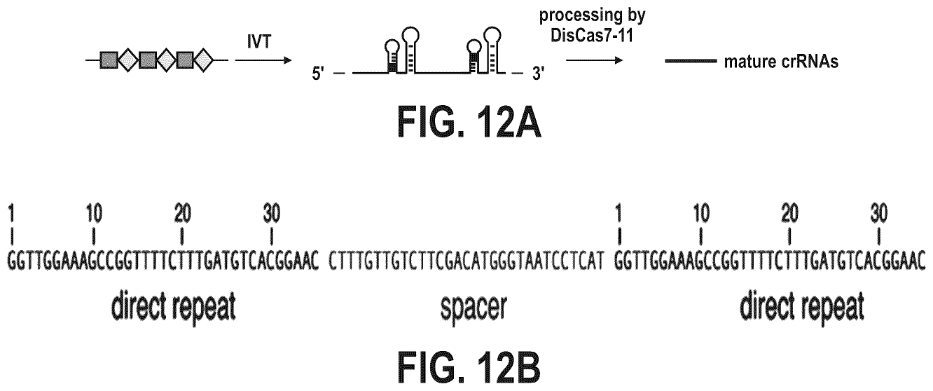

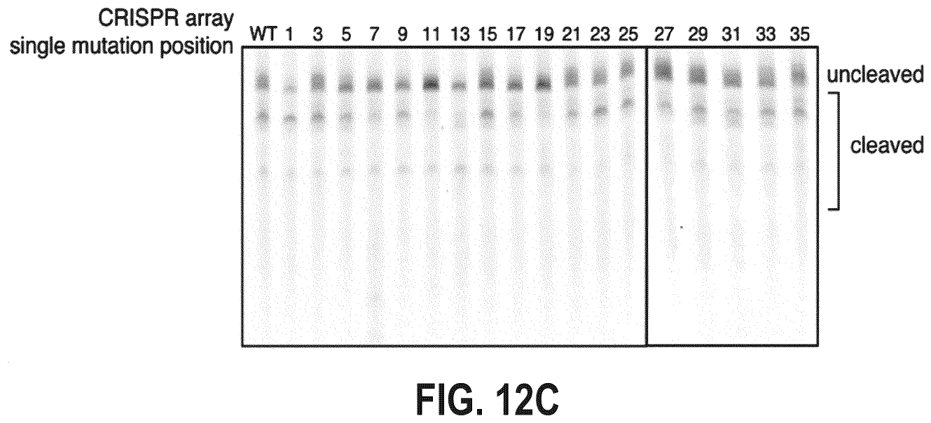

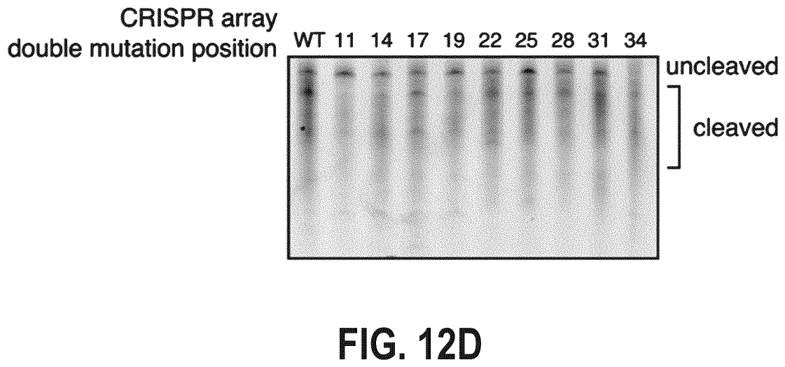

The robustness of DisCas7-11 pre-crRNA processing to DR mutations was evaluated. The in vitro transcription of pre-crRNA and processing by DisCas7-11 is illustrated in FIG. 12A. The sequence of a single spacer CRISPR array showing locations of mutated bases is shown in FIG. 12B. The transcribed pre-crRNA was processed and the results showed the effect of consecutive double mutations on processing (FIG. 12C). Transcribed pre-crRNA was processed and the results showed the effect of consecutive double mutations on processing (FIG. 12D).

### Example 12

### Effect of Ion on Cas7-11 Activity

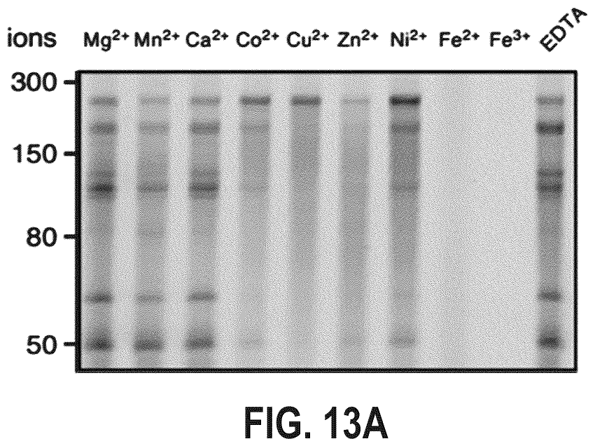

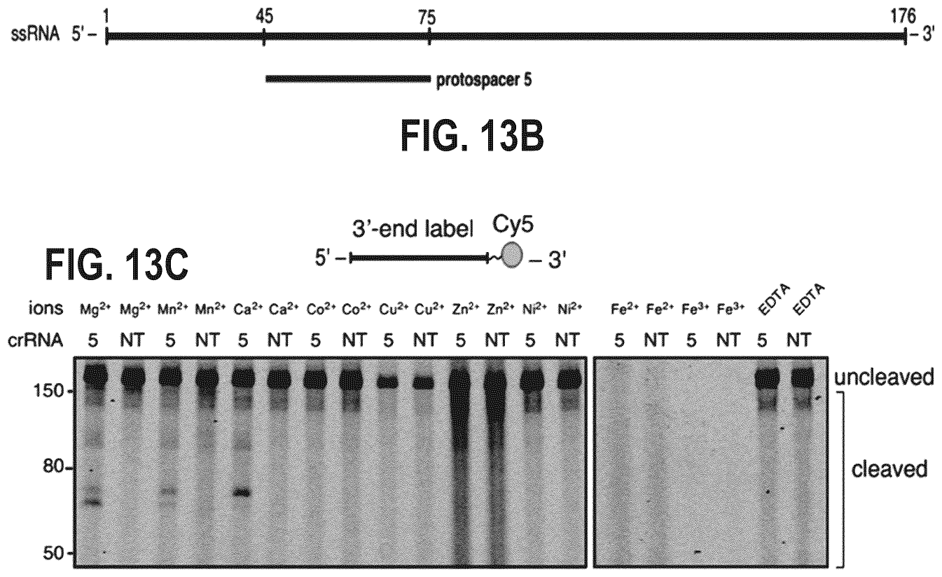

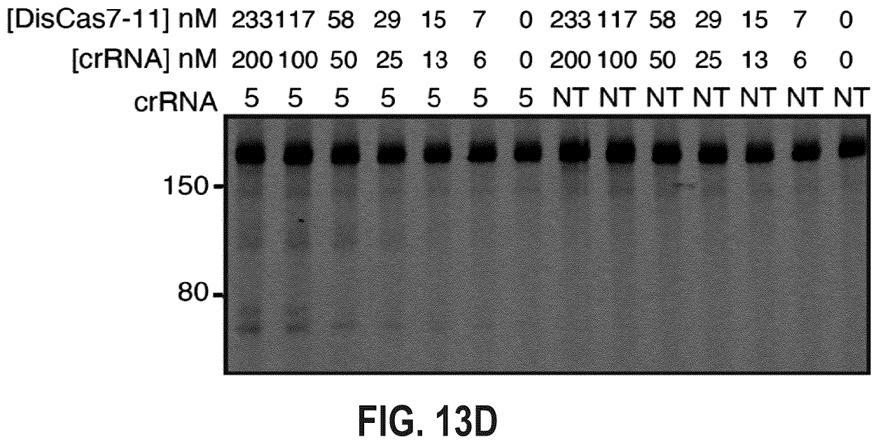

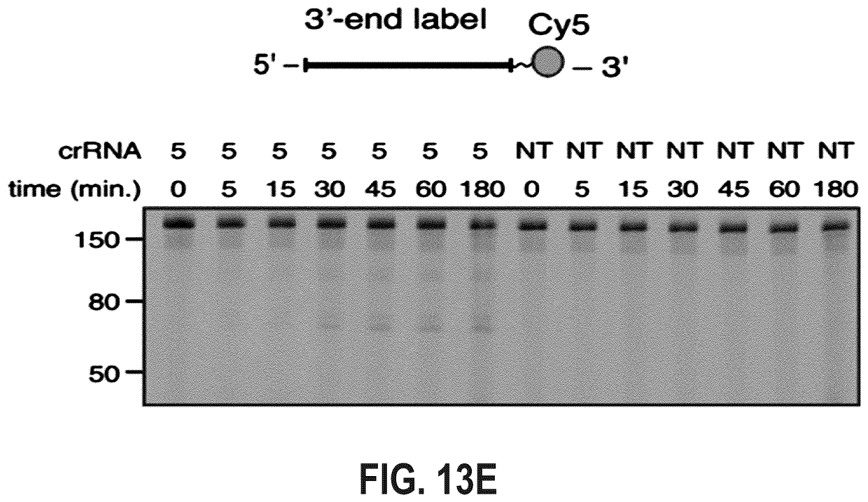

The effects of ions on pre-crRNA processing and ions, complex concentrations, and incubation time on target cleavage was analyzed. The in vitro transcription and 5′ end labeling of pre-crRNA and processing by DisCas7-11 were assessed. Pre-crRNA was processed by DisCas7-11 in the presence of different ions or chelating agents (FIG. 13A). CRISPR array processing by DisCas7-11 is shown to be ion independent. A schematic of in vitro cleavage of ssRNA target with DisCas7-11 and crRNA 5 is shown in FIG. 13B. The ssRNA target was incubated with DisCas7-11 and targeting or non-targeting crRNA in the presence of different ions or chelating agents (FIG. 13C). The results show the dependence on magnesium, manganese, and calcium. The cleavage of ssRNA target with increasing amounts of DisCas7-11-crRNA complex from 0 nM to 233 nM was performed (FIG. 13D). The cleavage of ssRNA target at increasing incubation periods from 0 minutes to 180 minutes was also performed (FIG. 13E).

### Example 13

### Effect of DR Length, Spacer Length and DR Mutations on Cas7-11 Activity

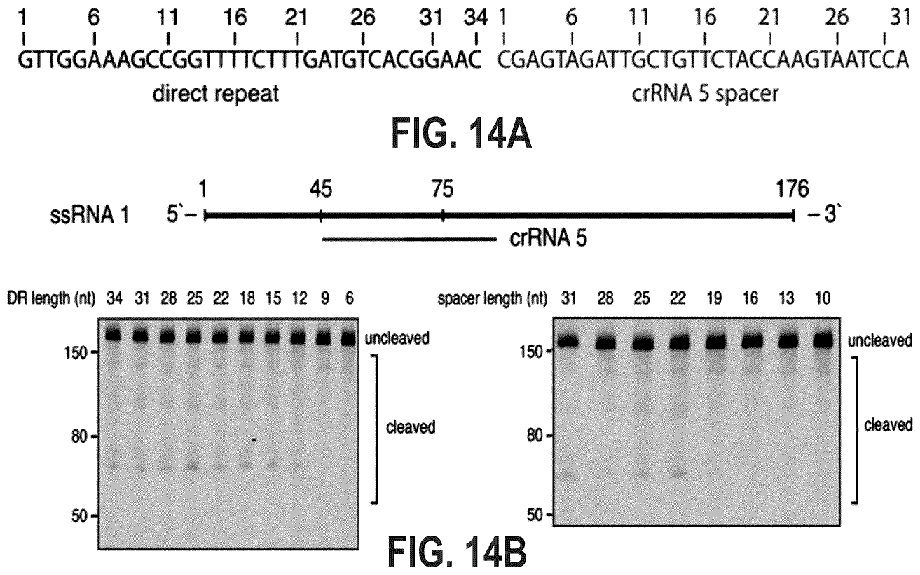

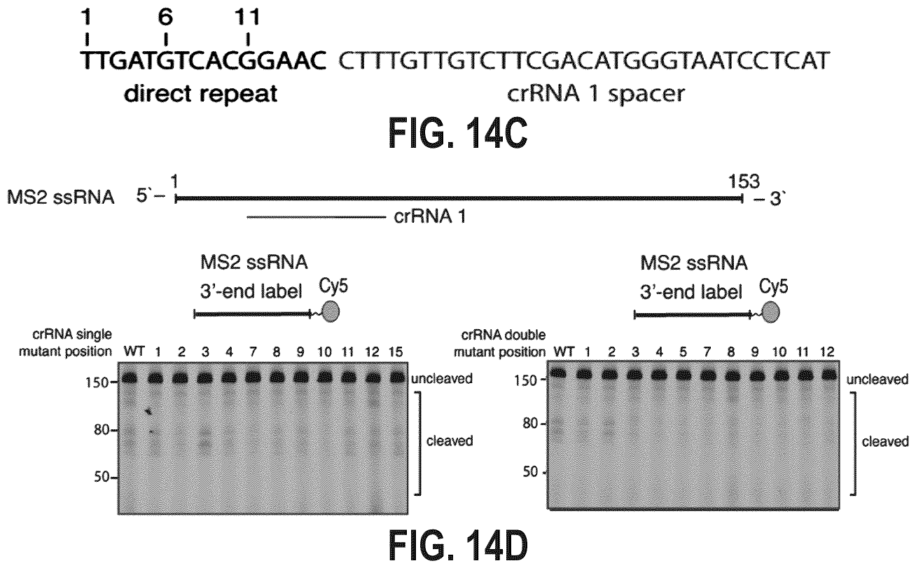

The effects of DR length, spacer length, and DR mutations on in vitro target cleavage by DisCas7-11 were evaluated. A schematic of the sequence of DisCas7-11 crRNA 5 targeting the ssRNA target is shown in FIG. 14A. The ssRNA target was cleaved with crRNA of varying DR and spacer lengths (FIG. 14B). A schematic of the sequence of DisCas7-11 crRNA 1 targeting the MS2 target is shown in FIG. 14C. The MS2 ssRNA target was cleaved (FIG. 14D). The results show the effect of single and double mutations at various positions in the DR, and several single and double mutations cause inhibition of DisCas7-11 target cleavage.

### Example 14

### DisCas7-11 In Vivo Collateral Activity

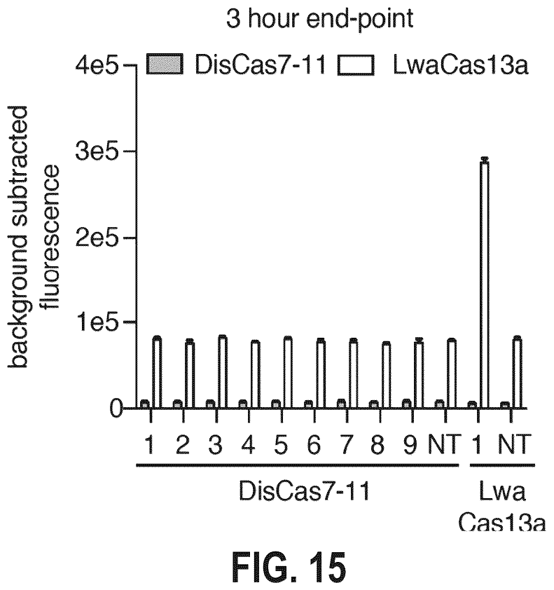

The in vivo collateral activity of DisCas7-11 was evaluated. The results from the three-hour end point fluorescence of RnaseAlert reporter incubated with either DisCas7-11 or LwaCas13a targeting and non-targeting guides against MS2 ssRNA target are shown in FIG. 15. The LwaCas13a demonstrates robust collateral activity whereas DisCas7-11 has no collateral cleavage of the RNase alert reporter.

### Example 15

### Activity of DisCas7-11 Catalytic Mutants

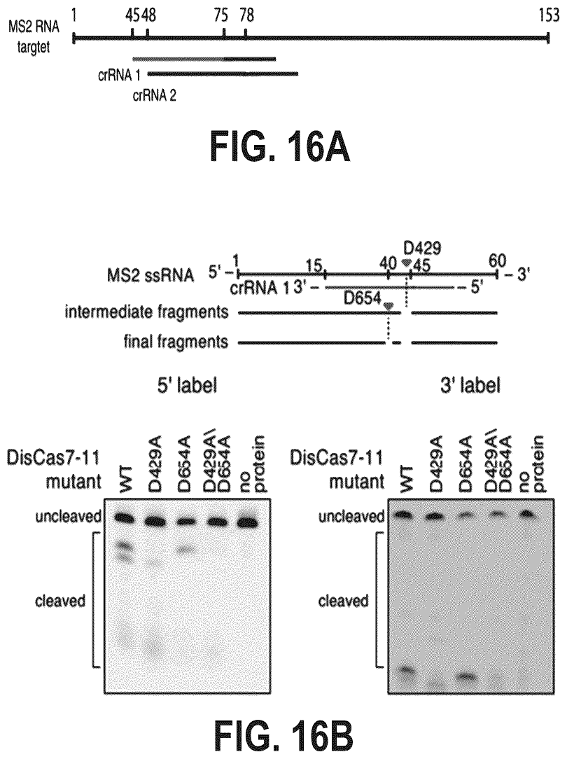

The cleavage activity of DisCas7-11 catalytic mutants on MS2 RNA target was evaluated. A schematic showing the positions of DisCas7-11 crRNA 1 and crRNA 2 targeting the MS2 target is shown in FIG. 16A. The cleavage of a synthetic MS2 ssRNA target with two cRNAs and wild type, D429A, D654A, and D429A/D654A DisCas7-11 proteins was performed (FIG. 16B). The D429A/D654A mutant demonstrates reduced cleavage activity on the target.

The DisCas7-11 catalytic mutants that were assessed are presented in Table 16 below.

TABLE 16

| Mutation | In vitro status | Domain |
|---|---|---|
| E70 | Purified, no effect | 1 |
| D77 | Purified, no effect | 1 |
| E93 | Purified, no effect | 1 |
| D111 | Purified, no effect | 1 |
| D143 | Purified, no effect | 1 |
| D160 | Purified, no effect | 1 |
| D162 | Purified, no effect | 1 |
| D177 | Purified, cut inactivated, | 1 |
| | bottom | |
| D185 | Purified, no effect | 1 |
| D193 | Purified, no effect | 1 |
| D226 | Purified, no effect | 1 |
| D238 | Purified, no effect | 1 |
| E280 | Purified, no effect | 2 |
| D287 | Purified, no effect | 2 |
| W315 | Purified, no effect | 2 |
| D316 | Purified, no effect | 2 |
| D379 | Purified, no effect | 2 |
| E381 | Purified, no effect | 2 |
| E390 | Purified, no effect | 2 |
| D422 | Purified, no effect | 3 |
| D429 | Purified, cut inactivated, | 3 |
| | middle (top shifts up) | |
| D437 | Purified, no effect | 3 |
| D454 | Purified, no effect | 3 |
| D487 | Purified, no effect | 3 |
| E497 | Purified, no effect | 3 |
| E520 | Purified, no effect | 3 |
| D543 | Purified, no effect | 3 |
| D581 | Purified, no effect | 3 |
| D642 | Purified, no effect | 4 |
| D654 | Purified, cut inactivated, | 4 |
| | bottom | |
| D727 | Purified, no effect | 4 |
| D733 | Purified, no effect | 4 |
| D740 | Purified, no effect | 4 |
| D745 | Purified, cut inactivated, | 4 |
| | bottom | |
| D753 | Purified, no effect | 4 |
| D758 | Purified, cut inactivated, | 4 |
| | bottom | |
| D781 | Purified, no effect | 4 |
| D798 | Purified, no effect | 4 |
| D825 | Purified, no effect | 4 |
| D909 | Purified, no effect | 5 |
| D914 | Purified, no effect | 5 |
| E959 | Purified, cut inactivated, | 5 |
| | bottom | |
| D970 | Purified, no effect | 5 |
| D982 | Purified, no effect | 5 |
| D998 | Purified, cut inactivated, | 5 |
| | bottom | |
| D1015 | Purified, no effect | 5 |
| D1025 | Purified, no effect | 5 |
| D1118 | Purified, no effect | 5 |
| D1179 | Purified, no effect | 5 |
| D1273 | Purified, no effect | 5 |
| D1297 | Purified, no effect | 5 |
| D1308 | Purified, no effect | 5 |
| D1367 | Purified, no effect | 5 |
| D1404 | Purified, no effect | 5 |
| D1539 | Purified, no effect | 5 |
| D1569 | Purified, no effect | 5 |
| D1588 | Purified, no effect | 5 |

### Example 16

### Cas7-11 EMSA Binding Assays

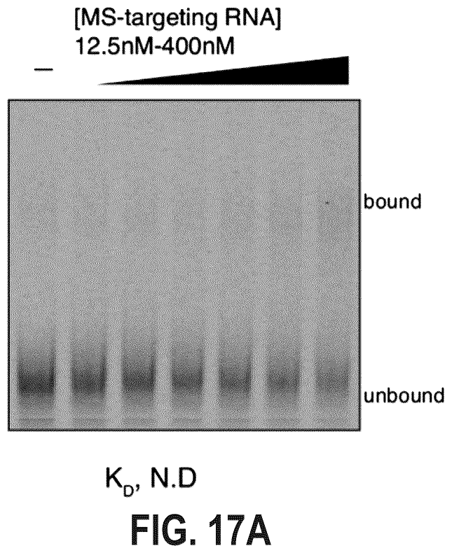

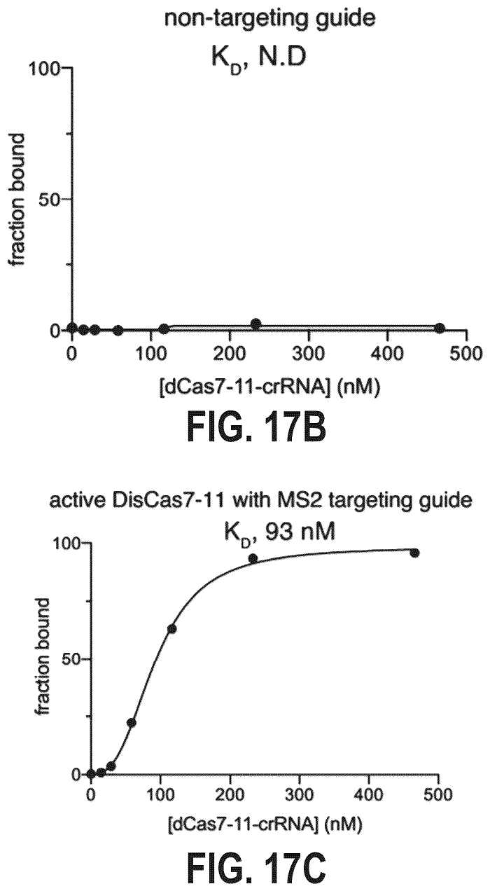

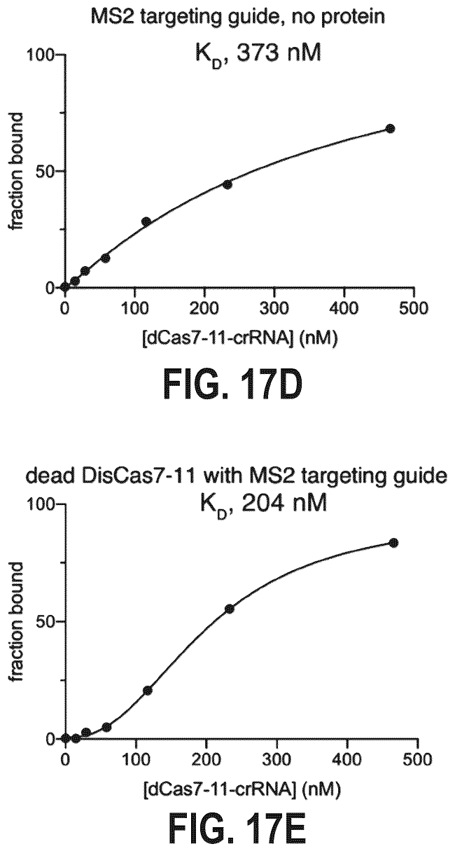

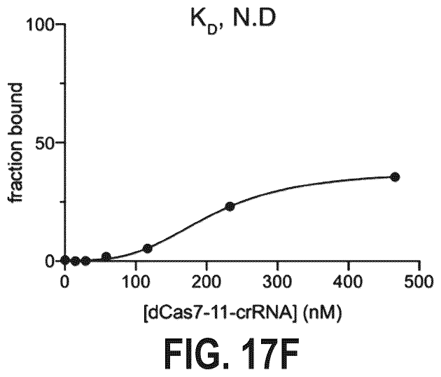

Electrophoretic mobility shift assays (EMSA) for crRNA-target interactions and quantification of EMSA intensities were performed (FIGS. 17A-F). The EMSA assays for targeting crRNA in the absence of DisCas7-11 show minimal binding of the cRNA and shift of the labeled target). Target binding was found to be crRNA-dependent, as expected, and the dead mutant to have a similar affinity to ssRNA as the active DisCas7-11 protein. Furthermore, agreeing with the lack of ssDNA cleavage, DisCas7-11 showed weak ssDNA binding, and efficient binding required the full protein complexed with a.

### Example 17

### DisCas7-11 Cleavage Sites on ssRNA Targets

The characterization of DisCas7-11 cleavage sites on ssRNA targets was performed.

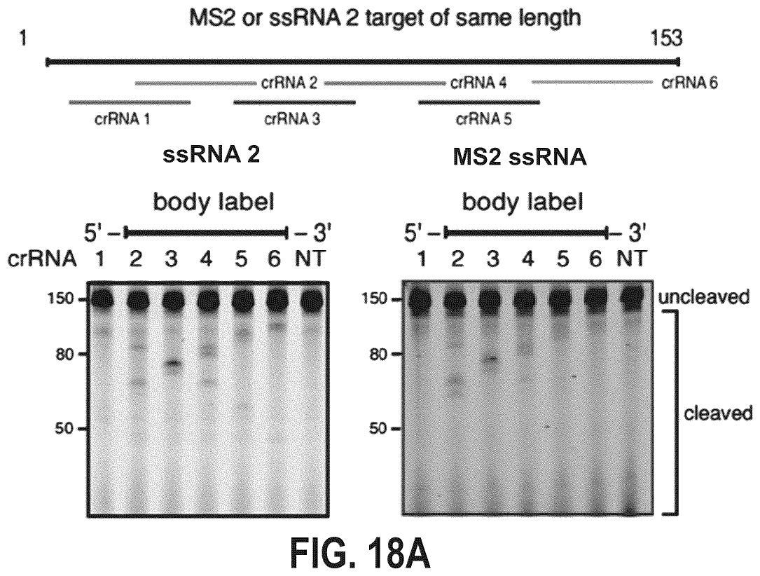

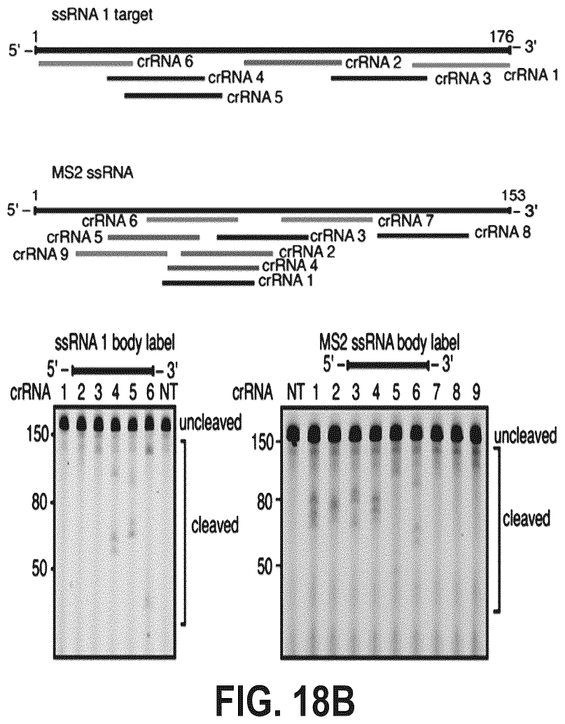

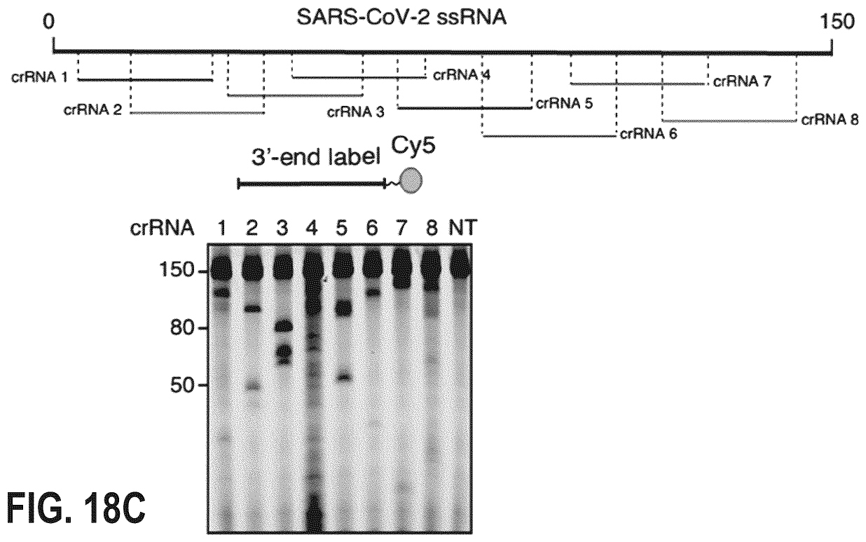

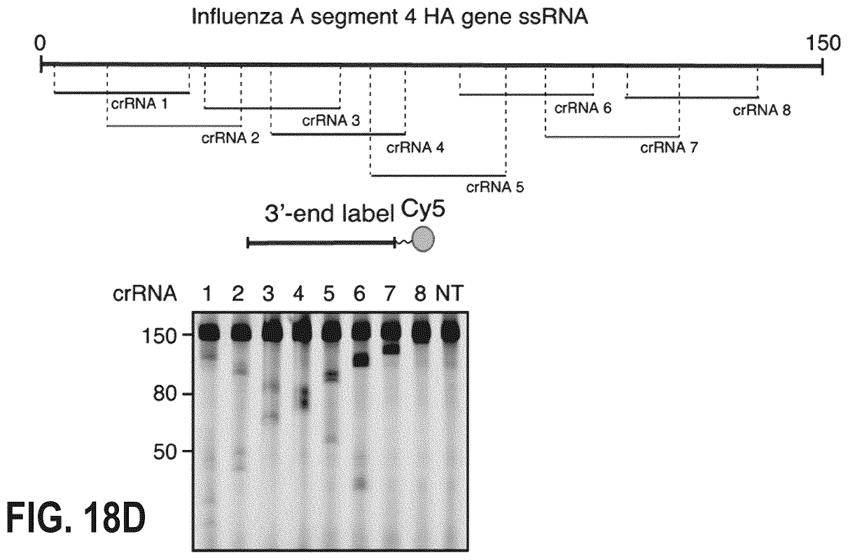

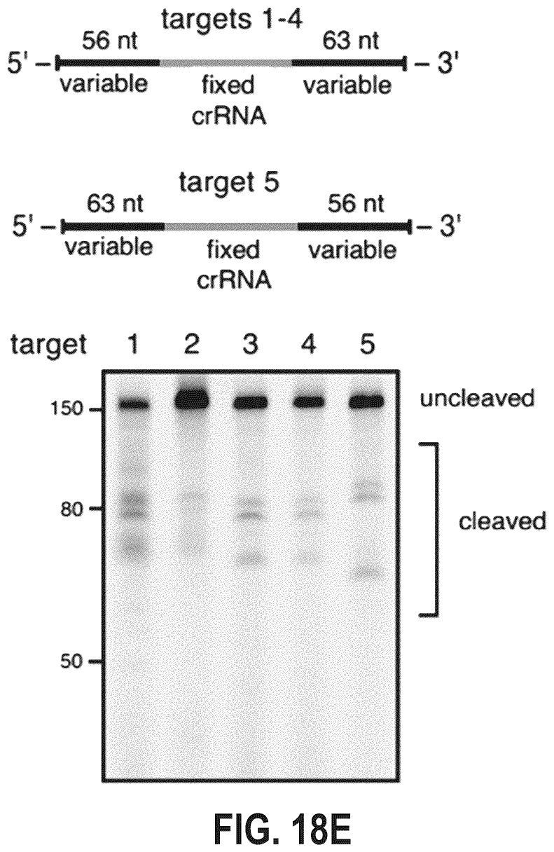

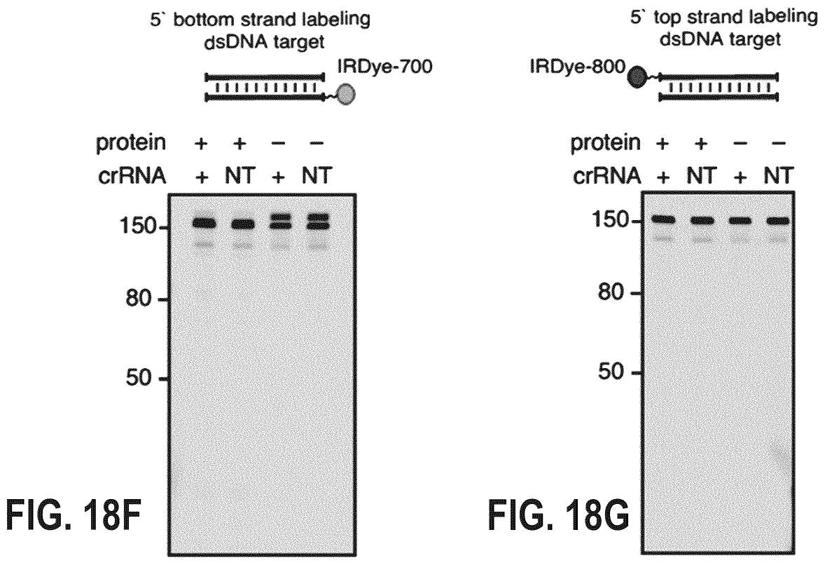

DisCas7-11 was incubated with a panel of crRNAs targeting either MS2 ssRNA or ssRNA target 2, two targets of equivalent length (153 nt) (FIG. 18A). The crRNAs are designed to target sites at the same position on both targets in order to better understand whether sequence-specific cleavage preferences exist and whether cleavage cut sites are position dependent. The cleavage of ssRNA 1 and MS2 ssRNA at multiple sites with tiled crRNAs were also performed (FIG. 18B). A varying cleavage pattern based on target position was observed.

A schematic of the position of the tested crRNAs along the SARS-CoV-2 ssRNA is shown in FIG. 18C. The results from the cleavage of synthetic SARS-CoV-2 ssRNA at multiple sites with tiled crRNAs showing a varying cleavage pattern based on target position are also shown in the same figure. A schematic of the position of the tested crRNAs along the Influenza A ssRNA target is shown in FIG. 18D. The results from the cleavage of synthetic influenza A segment 4 HA gene ssRNA at multiple sites with tiled crRNAs showing a varying cleavage pattern based on target position are also shown in the same figure.

DisCas7-11a was incubated with a crRNA targeting a crRNA site flanked by different sequences (FIG. 18E). While the spacer:target complementarity region is fixed, the sequences around the site are completely variable, allowing determination of any sequence preferences for cleavage. DisCas7-11a was also incubated with a crRNA targeting a double-stranded (dsDNA) target with 5′ labeling of the bottom strand (FIG. 18F) or top strand (FIG. 18G).

### Example 18

### Cleavage Activity on Various Length EGFP ssRNA Substrates

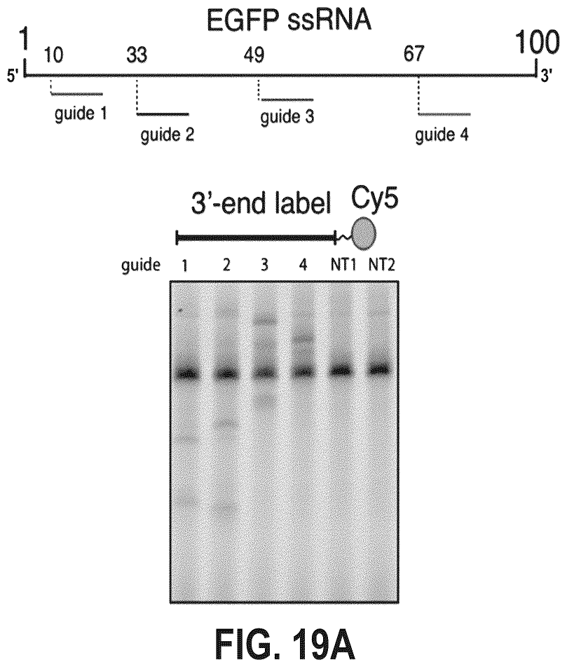

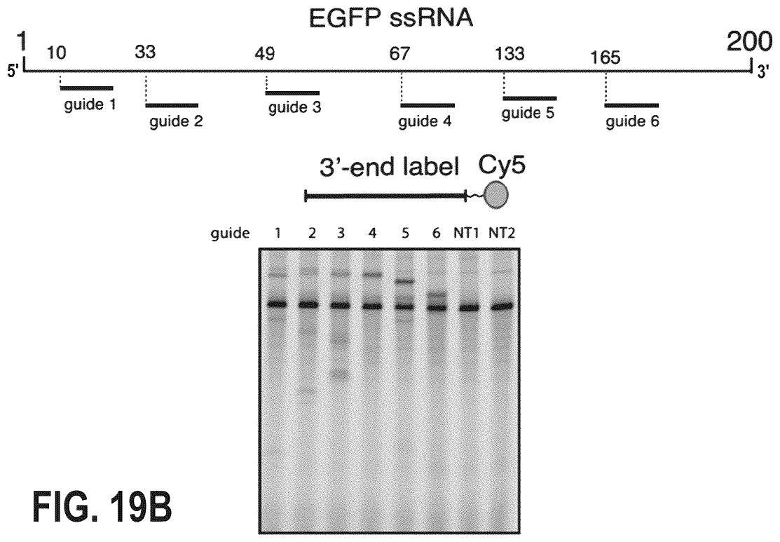

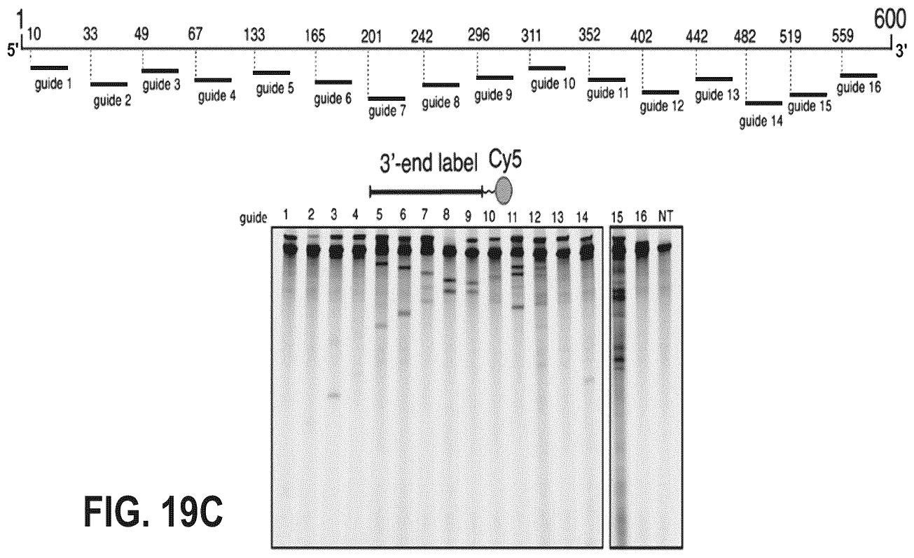

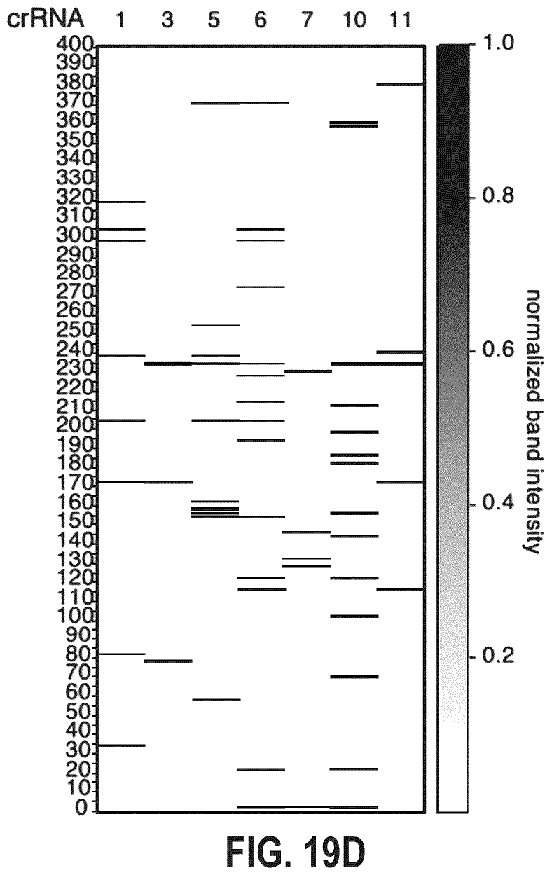

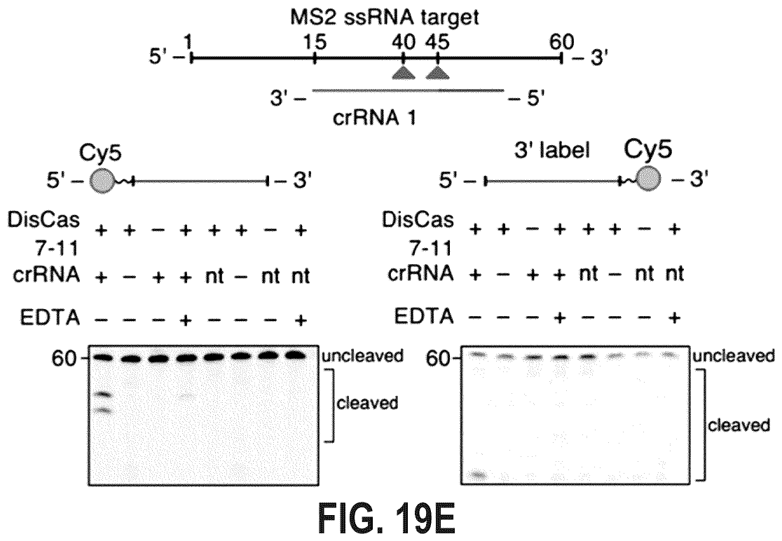

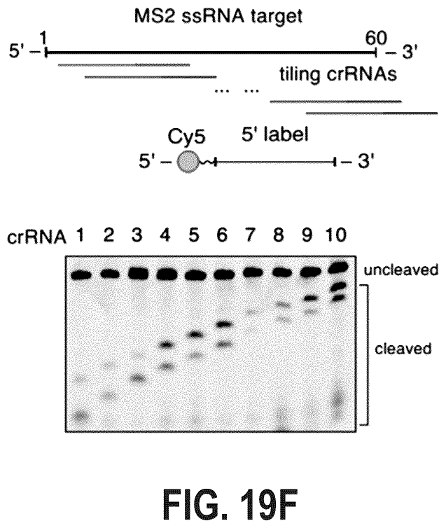

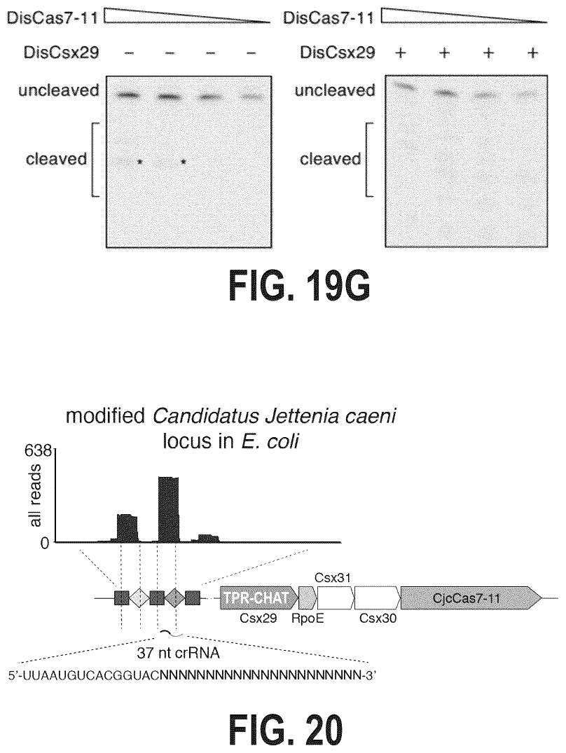

Biochemical characterization of DisCas7-11 programmable RNA cleavage activity on various length EGFP ssRNA substrates was performed. A schematic of the position of the tested crRNAs along the 100 nt EGFP ssRNA target is shown in FIG. 19A. The biochemical characterization of programmable DisCas7-11 RNA cleavage of 100 nt long and 3′ fluorescently labeled EGFP ssRNA target with tiling crRNAs is also shown in this figure. A schematic of the position of the tested crRNAs along the 200 nt EGFP ssRNA target is shown in FIG. 19B. The biochemical characterization of the programmable DisCas7-11 RNA cleavage of 200 nt long and 3′ fluorescently labeled EGFP ssRNA target with tiling crRNAs is also shown in this figure. A schematic of the position of the tested crRNAs along the 600 nt EGFP ssRNA target is shown in FIG. 19C. The biochemical characterization of the programmable DisCas7-11 RNA cleavage of 600 nt long and 3′ fluorescently labeled EGFP ssRNA target with tiling crRNAs is also shown in this figure. The DNA sequencing analysis of selected EGFP targeting crRNA conditions on an unlabeled 400 nt EGFP ssRNA target was performed (FIG. 19D). The DisCas7-11a cleavage was found to occur at both sides directly flanking the crRNA binding site, with cleavage occurring approximately 20 bases downstream of the 5′ position of the spacer. Other biochemical characterizations of DisCas7-11 programmable RNA cleavage activity are shown in FIGS. 19E-19G

### Example 19

### Candidatus Jettenia caeni Type III-E CRISPR-Cas system

The heterologous expression of the Candidatus Jettenia caeni Type III-E CRISPR-Cas system and associated CRISPR array in E. coli was performed. The schematic of Candidatus Jettenia caeni locus (minus Cas1) in E. coli is shown in FIG. 20. The processing of crRNAs occurs when the CjcCas7-11 locus is heterologously expressed in E. coli, and mature 37 nt crRNAs are generated containing a 15-nt DR.

### Example 20

### Processing of Cas7-11 orthologs

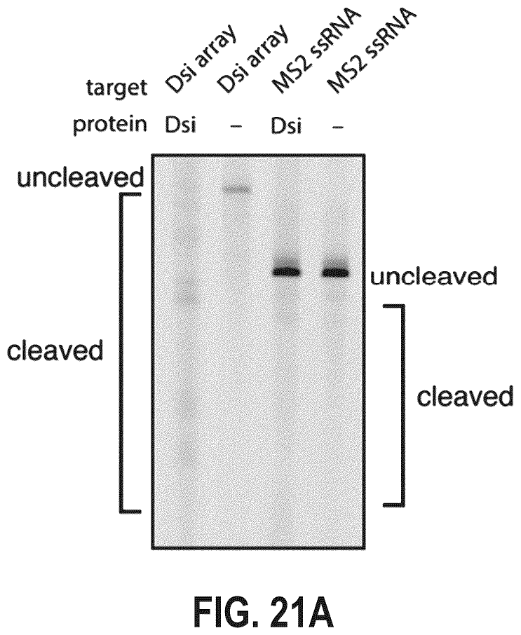

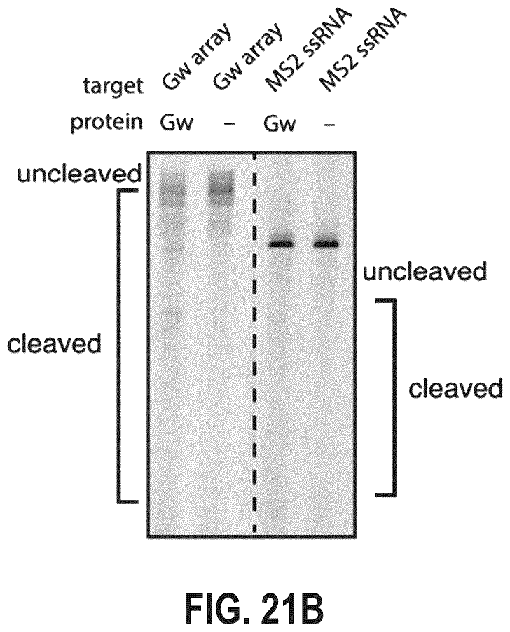

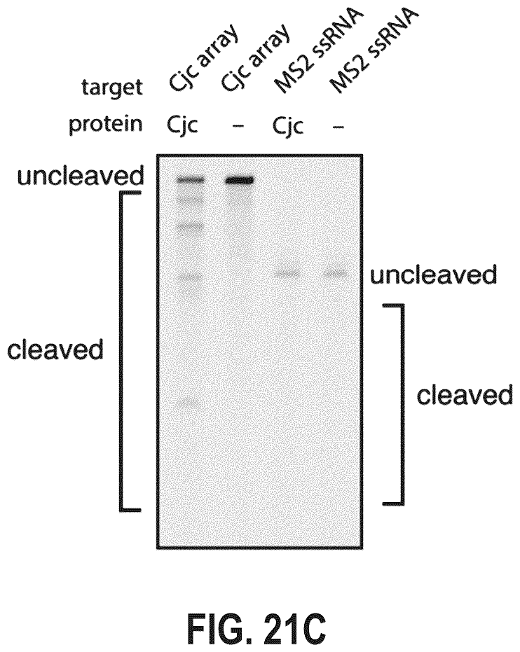

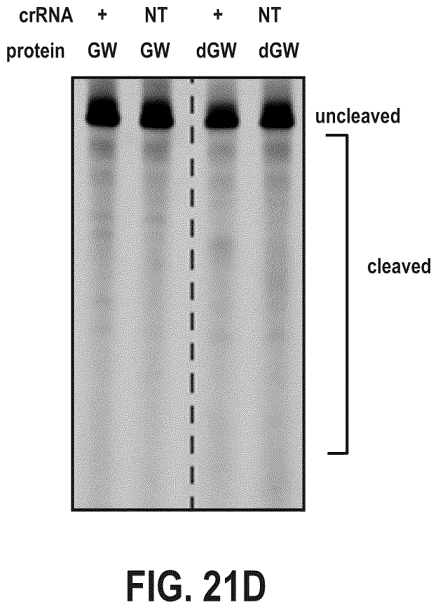

The processing of Cas7-11 orthologs was found to be specific to the cognate pre-cRNAs and to do not occur on other synthetic RNAs. The DisCas7-11 pre-cRNA processing is specific to the DisCas7-11 array and does not occur on the MS2 ssRNA target as illustrated in FIG. 21A. The GwCas7-11 pre-cRNA processing is specific to the GwCas7-11 array and does not occur on the MS2 ssRNA target as illustrated in FIG. 21B. The CjcCas7-11 pre-CRNA processing is specific to the CjcCas7-11 array and does not occur on the MS2 ssRNA target as illustrated in FIG. 21C. FIG. 21D shows the comparison of target cleavage between active CjcCas7-11 and dead CjcCas7-11.

### Example 21

### Cas7-11 Correction of Cluc W85X

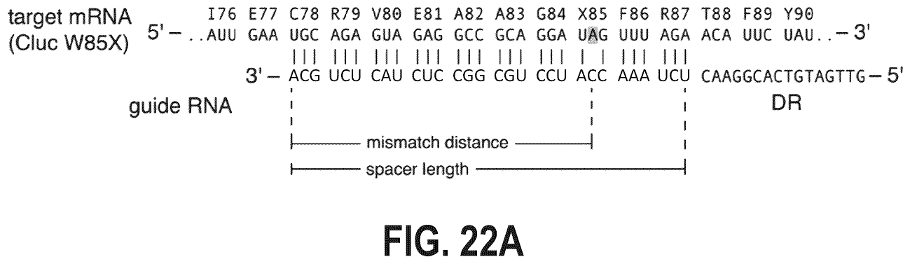

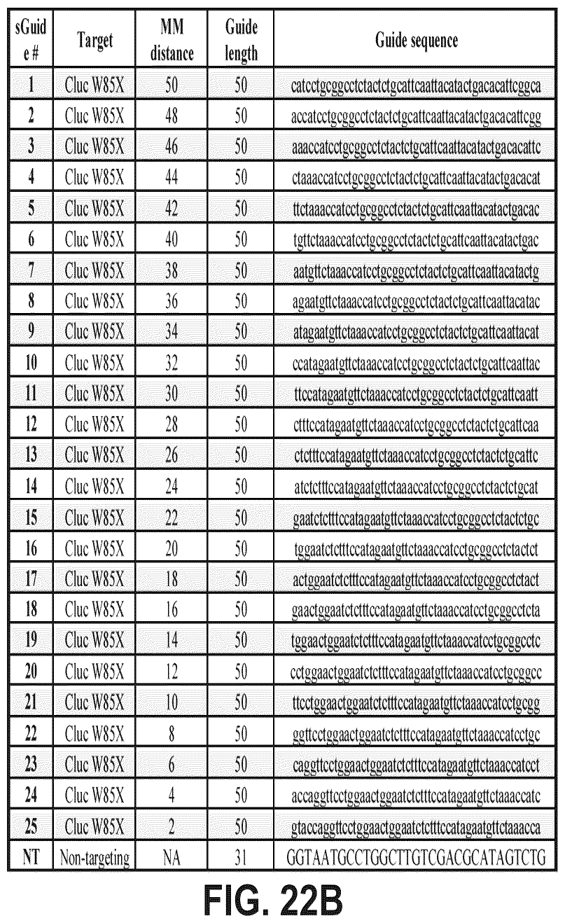

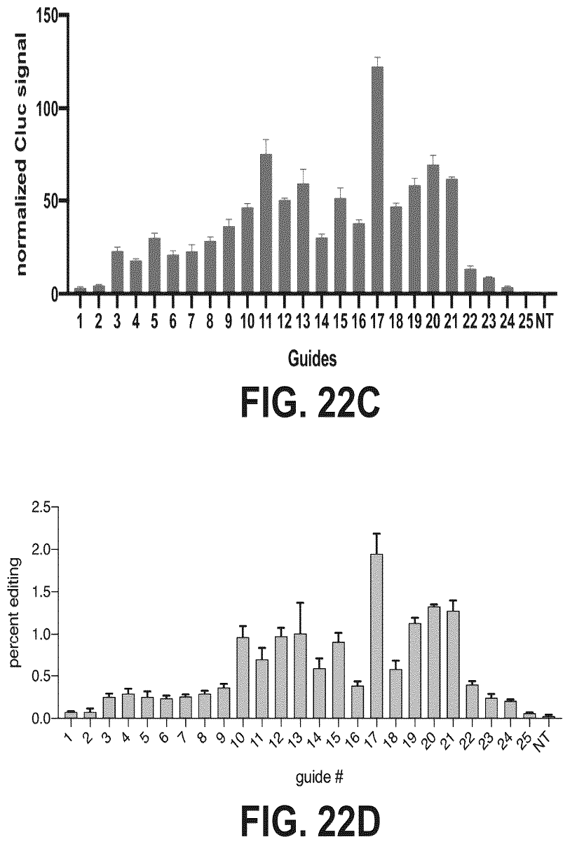

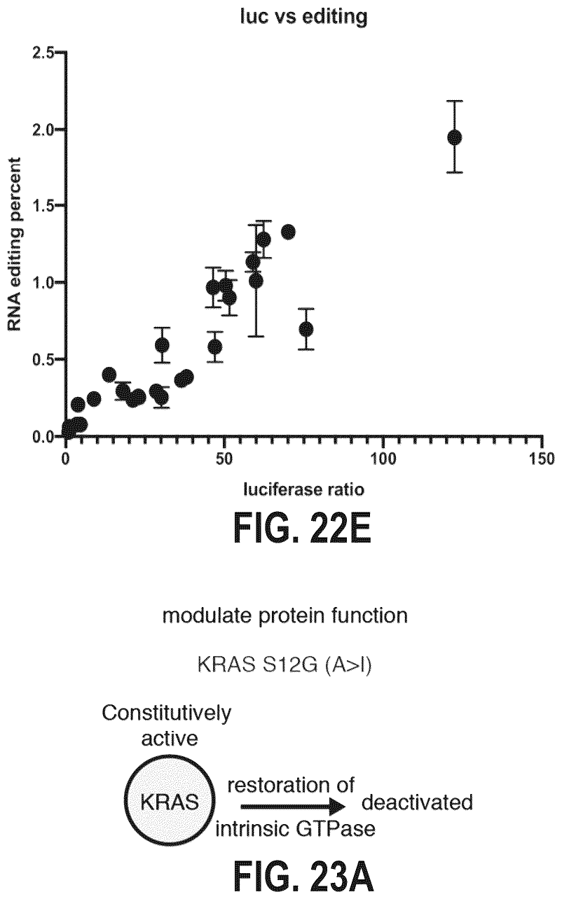

The correction of Cluc W85X mRNA by dDisCas7-11 was measured. A schematic of the DisCas7-11 guide design for RNA editing of Cluc W85A target mRNA is shown in FIG. 22A. A table disclosing the different guides used for the correction of the mRNA is shown in FIG. 22B. The dDisCas7-11 correction of Cluc W85X RNA by the different guides is shown in FIG. 22C, and the percentage editing for the correction is shown in FIG. 22D. The luciferase correction that correlates to the correction percent is shown in FIG. 22E.

### Example 22

### Harvesting and Next-generation Sequencing of crRNA and pre-crRNA From Desulfonema ishimotonii and E. coli

To extract total RNA, freeze-dried Desulfonema ishimotonii cultures (DSMZ 9680) were resuspended in TRIzol (R2051, Zymo), homogenized by bead beating with zirconia/silica beads, and processed with the Direct-Zol RNA miniprep protocol (R2051, Zymo). Purified RNA samples were treated with T4 Polynucleotide Kinase (M0201S, New England Biolabs), both with and without ATP to allow for the enrichment of 3′-P and 5′-OH ends. Samples has ribosomal RNA depleted using the Ribo Minus Kit (K155004, Thermo Fisher Scientific) before being processed for next-generation sequencing with the NEBNext Small RNA Library Prep Set for Illumina sequencing (E7330S, New England Biolabs). Notably, the PCR extension step was increased to 1 minute to allow for longer templates to be included in the library. Libraries were sequenced on an MiSeq (Illumina) to sufficient depth and analyzed using the alignment tool BWA (Li and Durbin 2009). Paired-end alignments were used to extract entire transcript sequences using Galaxy tools (https://usegalaxy.org/), and these sequences were analyzed using Geneious 8.1.5 (Biomatters, Auckland, New Zealand) and custom scripts (https://github.com/abugoot-lab).

### Example 23

### Design and Cloning of Bacterial Constructs

To clone DisCas7-11 constructs, genomic DNA from D. ishimotonii cultures (DSMZ 9680) was extracted using the Blood & Cell Culture DNA Mini Kit (13323, Qiagen). The Type III-E D. ishimotonii CRISPR locus was PCR amplified and cloned into a pACYC184 backbone with chloramphenicol resistance using Gibson cloning. For the Cas7-11 only construct, DisCas7-11a was cloned with a J23119 promoter and B0015 terminator. Removal or addition of other genes in the CRISPR locus was cloned using Gibson cloning; when adding genes to the Cas7-11-only construct, expression of these genes was driven by a pLac promoter. In order to facilitate simple cloning of spacers, a minimal spacer construct with two flanking direct repeats (DR) surrounding a Golden Gate acceptor site was used as a backbone for Golden Gate cloning (Engler and Marillonnet 2014). To clone the CjcCas7-11b locus, DNA corresponding to the locus was synthesized by GeneArt (Thermo Scientific). Unless otherwise noted, all clonings were transformed into Stbl3 cells (C737303, Thermo Fisher Scientific) made competent with the Mix and Go kit (T3001, Zymo Research), all colonies were picked into Terrific Broth (TB) (24 g/L Yeast Extract, 12 g/L Tryptone, 9.4 g/L K2HPO4, 2.2 g/L KH2PO4, pH 7.2) supplemented with the appropriate antibiotic (chloramphenicol, 25 μg/mL; ampicillin, 100 μg/mL) and all plasmids were isolated using the QiaPrep Spin Miniprep kit (27104, Qiagen) protocol and verified by next generation sequencing on a MiSeq (Illumina). All bacterial plasmids used are listed in Table 3.

### Example 24

### Cloning of Screening Libraries for MS2 Interference Activity Screen

To design the MS2 library for screening, all possible spacers targeting the MS2 genome were computationally extracted, and synthesized as a library by Twist Biosciences. This library was Golden Gate cloned into the minimal CRISPR array containing the Golden Gate acceptor site on the DisCas7-11a-only backbone and transformed into Endura Duo electro-competent cells (60240-1, Lucigen) and plated on chloramphenicol-supplemented Bioassay agar plates. 16 hours post transformation, libraries were harvested by scraping and purified using a NucleoBond Xtra MaxiPrep EF (740424.10, Takara Bio).

### Example 25

### Bacterial Phage Interference PFS Screen Assay

To transform the MS2 spacer library, 400 uL NovaBlue(DE3) Gigasingle competent cells (71227-3, EMD Millipore) were transformed with a total of 2 ug of the spacer library. Cells were recovered in SOC while shaking at 37° C. for 1 hour. After recovery, cells were split into two biological replicates and used to inoculate 8 mL of Luria Broth (LB) (10 g/L tryptone, 5 g/L yeast extract, 5 g/L NaCl) supplemented with chloramphenicol. Cells were further incubated in LB-chlor while shaking at 37° C. for 4 hours, after which the volume was brought to a total of 12 mL for each biological replicate. Replicates were split into 6 2 mL tubes, and phages of different dilutions were added to each tube. Cells were incubated with phage while shaking at 37° C. for 3 hours, after which they were plated on Bioassay plates with LB-agar supplemented chloramphenicol. After 16 hours of growth, all colonies were harvested by scraping and scraped and plasmid DNA was extracted with the NucleoBond Xtra MaxiPrep EF (740424.10, Takara Bio). Spacer sequences were PCR amplified from extracted plasmids and sequenced using a MiSeq (Illumina) with a paired-end 150 cycle kit.

### Example 26

### Cloning of Libraries and Screening for Beta-Lactamase and Transcribed/Non-Transcribed PFS Screens

Plasmid libraries for PFS screens were cloned from synthesized oligonucleotides (IDT) consisting of 7 randomized nucleotides either upstream or downstream of the spacer 1 target. To generate dsDNA fragments for cloning, the ssDNA oligonucleotides were annealed to a short primer for second strand synthesis by large Klenow fragment (M0210S, New England Biolabs). dsDNA fragments were Gibson cloned into digested pUC19, either at the 5′-end of the beta-lactamase (Ampicillin resistance) transcript (RNA targeting library), or in a non-transcribed region of pUC19 (DNA targeting library). Gibson clonings were electroporated into Endura Duo electro-competent cells (60240-1, Lucigen) and plated on Bioassay plates with LB-agar supplemented with ampicillin. After 16 hours of growth, all colonies were harvested by scraping and scraped and plasmid DNA was extracted with the NucleoBond Xtra MaxiPrep EF (740424.10, Takara Bio).

To screen libraries, we co-transformed 50 ng of the pooled ampicillin library and an equimolar amount of the Cas7-11 locus plasmid or pACYC184 plasmid control intoNovaBluc(DE3) Gigasingle competent cells (71227-3, EMD Millipore). After transformation, cells were plated on ampicillin and chloramphenicol to select for both plasmids. After 16 hours of growth, all colonies were harvested by scraping and scraped and plasmid DNA was extracted with the NucleoBond Xtra MaxiPrep EF (740424.10, Takara Bio). The target PFS region was PCR amplified and sequenced using a MiSeq (Illumina) with a single-end 150 cycle kit.

### Example 27

### Computational Analysis of in Vivo Screens

To determine enriched spacers from the bacteriophage interference screens, sequenced spacer regions were counted and normalized to total reads for each sample. For enriched spacers, enrichment was measured as the log 2 ratio compared to no phage dilution controls, with a pseudocount adjustment. PFS regions (both at 5′ and 3) from spacers above a 1.7 log 2 enrichment threshold in both biological replicates were used to generate sequence logos for the phage dilution samples.

For transcribed/non-transcribed (beta-lactamase) pUC19 PFS screens, PFS regions were extracted and computationally collapsed to 5 nt to have broader coverage. Collapsed PFS were counted and normalized to total reads for each sample. For a given PFS region, enrichment was measured as the log ratio compared to control (pACYC184 control), with a 0.01 pseudocount adjustment. PFSs above a 6 depletion threshold that were enriched in both biological replicates were collected and used to generate sequence logos (Crooks et al. 2004).

### Example 28

### Bacterial Phage Interference Assay for Individual Spacers

To test individual spacers for MS2 interference with drop plaque assays, complementary oligonucleotides encoding the spacer sequences (see Table 4) flanked by overhangs corresponding to the Cas7-11a Golden Gate acceptor sites were ordered from IDT. Oligonucleotides (final concentration 10M) were annealed in T4 ligase buffer (B0202S, New England Biolabs) supplemented with 5 units of T4 Polynucleotide Kinase (M0201S, New England Biolabs). The oligonucleotides were phosphorylated at 37° C. for 30 minutes and annealed by heating to 95° C. for 5 minutes followed by a 5° C./minute cool to 25° C. Annealed oligos were then cloned into the locus backbone by Golden Gate cloning. After verification by sequencing, clonal plasmids were transformed into NovaBlue(DE3) GigaSingle competent cells (71227-3, EMD Millipore) made competent with the Mix and Go kit (T3001, Zymo Research). Transformed GigaSingle cells were seeded from an overnight culture grown to OD600 of ˜2, at which point they were diluted 1:6 in Top Agar (10 g/L tryptone, 5 g/L yeast extract, 10 g/L NaCl, 7 g/L agar) and poured onto warm LB-chloramphenicol plates. Dilutions of MS2 phage in phosphate buffered saline were then spotted on the plates with a multichannel pipette, and plaque formation was recorded after overnight incubation.

### Example 29

### RFP Targeting and Growth Assay

Spacers (see Table 4) targeting RFP mRNA were Golden Gate cloned into the DisCas7-11a-only construct (as described above). After verification by sequencing, clonal plasmids were co-transformed with a tetracycline-inducible RFP plasmid on the pBR322 backbone (Addgene 79157). Colonies were picked into TB supplemented with ampicillin, chloramphenicol, and 50 ng/mL anhydrotetracycline final (631310, Takara Bio) and grown to saturation by shaking at 37° C. overnight. Cells were separated by centrifugation through a 40 μm filter plate (MANMN4010, EMD Millipore) and analyzed by flow cytometry on a FACSCanto II Cell Analyzer (BD Biosciences). Knockdown efficiency was quantified as the percent of RFP positive cells compared to a non-targeting spacer control.

### Example 30

### Cas7-11 Protein Purification

The E. coli codon-optimized genes for DisCas7-11a and GwCas7-11c, and the human codon-optimized gene for CjcCas7-11b were synthesized by Twist Biosciences, PCR amplified, and Gibson into a Twin-Strep-tag and SUMO tag bacterial expression vector (Addgene 90097). Sequence verified clonal Cas7-11 expression constructs were transformed into either T7 Express lysY Competent E. coli cells (C3010I, New England Biolabs), for DisCas7-11a and GwCas7-11c, or Rosetta(DE3)pLysS Competent Cells (70956-3, EMD Millipore), for CjcCas7-11b. Colonies were picked and grown overnight in TB supplemented with ampicillin. 10 ml of overnight culture was used to inoculate 1 L of TB supplemented with ampicillin, and the culture was grown by shaking at 37° C. until the OD600 reached 1. At this point, cultures were cooled at 4° C. for 30 minutes, and induced with IPTG (16758, Sigma) to a final concentration of 500 uM. Induced cultures were grown overnight (16-24 hours) by shaking at 18° C. After growth, Cells were harvested by centrifuged at 17,700×g for 15 min at 4° C., and the cell pellet was frozen at −80° C. for later purification.

Frozen cell paste was crushed and resuspended to a final concentration of 250 mg/mL in lysis Buffer (20 mM Tris-HCl, 500 mM NaCl, 1 mM DTT, pH 8.0) supplemented with Complete Ultra EDTA-free protease inhibitor tablets (5892953001, Sigma), 1 mg/mL lysozyme (L6876, Sigma) and 1U/mL Benzonase nuclease (E1014, Sigma). Resuspension was performed by spinning for 30 minutes at 4° C. After resuspension, the solution was sonicated on ice with a 600W Ultrasonic Homogenizer (U.S. Solid) for 15 minutes with a 50% duty cycle. Sonicated lysate was cleared by centrifugation at 38,400×g for 1 hour at 4° C. The supernatant was supplemented with 1 mL of Strep-Tactin XT superflow high capacity resin (2-4030-010, IBA Life Sciences) and incubated with rotation for 1 hour at 4° C. Bound resin was loaded onto a glass Econo-Column (7371507, Bio-Rad) and washed with three column volumes of lysis buffer supplemented with Complete Ultra EDTA-free protease inhibitor tablets. To cleave protein off of bead-bound Twin-Strep-tag and SUMO tag, the resin was resuspended in 3 mL of SUMO digest buffer (30 mM Tris-HCl, 500 mM NaCl, 1 mM DTT, 0.15% NP-40, pH 8.0) supplemented with 100 ug of SUMO protease (purified in-house). Cleavage occurred on-column overnight at 4° C. with rotation, after which supernatant was drained from the column by gravity filtration and concentrated to a volume of 1 mL with a 100 kDa centrifugal filter (UFC810024, Sigma). Concentrated protein was loaded onto a gel filtration column (Superdex 200 Increase 10/300 GL, GE Healthcare Life Sciences) via an ÄKTA pure FPLC machine. The resulting fractions from gel filtration were analyzed by SDS-PAGE Stain-Free gels (4568096, Bio-Rad), and fractions containing protein were pooled, buffer exchanged into Storage Buffer (600 mM NaCl, 50 mM Tris-HCl pH 7.5, 5% glycerol, 2 mM DTT), quantified via comparison SDS-PAGE with a Bovine Serum Albumin (BSA) ladder, aliquoted, and frozen at −80° C. for storage.

### Example 31

### Nucleic Acid Target and Cas7-11 Pre-crRNA Preparation

RNA targets for testing cleavage and pre-crRNA targets were synthesized by in vitro transcription. Single stranded DNA oligonucleotide templates with a T7 RNA polymerase promoter appended at the 5′ end were synthesized by IDT, and converted to dsDNA by PCR and gel extraction. Gel extracted dsDNA products (˜1 μg/reaction) were used as input for the HiScribe T7 Quick High Yield RNA Synthesis kit (E2050S, New England Biolabs), with the 30 uL reaction size recommended for transcripts <300 nt. Transcription occurred overnight at 30° C., after which targets were purified with RNA Clean and Concentrator columns (R1017, Zymo Research). Labeling was performed with the 3′ EndTag End Labeling System (MB-9002, Vector Laboratories) and 5′ EndTag End Labeling System (MB-9001, Vector Laboratories), per the manufacturer's instructions, which was followed by incubation with 100× molar ratio of Cyanine5.5 maleimide (17080, Lumiprobe) at 65° C. for 30 minutes. We confirmed that this labeling was not 5′ or 3′ specific, but rather body labeling, via Rnase H gel assays (fig. S21), likely due to the intercalating or groove-binding properties of Cyanine5.5 (Biver et al. 2005). After labeling, RNA was purified with RNA Clean and Concentrator columns (R1017, Zymo Research). All targets used for in vitro cleavage are listed in supplemental Table 5.

### Example 32

### Cas7-11 crRNA Preparation

crRNA designs were ordered from IDT as reverse complement DNA oligonucleotide templates with a T7 RNA polymerase promoter appended at the 5′ end. These templates were annealed with a T7 promoter oligonucleotide, generating a dsDNA promoter region. Annealing occurred in Taq Buffer (B9014S, New England Biolabs) with both oligonucleotides at 10 μM. 1 uL of the anneal reaction was used as input for the HiScribe T7 Quick High Yield RNA Synthesis kit (E2050S, New England Biolabs), with the 30 uL reaction size recommended for transcripts <300 nt. Transcription occurred overnight at 37° C., after which targets were purified with RNAClean XP beads (A63987, Beckman Coulter); beads were used at 3.3× volume ratio and supplemented with 3× volume of isopropanol. All crRNA used for in vitro cleavage are listed in supplemental Table 6.

### Example 33

### In vitro Nuclease Assays

Unless otherwise indicated, in vitro nuclease assays were performed with 233 nM purified Cas7-11, 30 nM of labeled ssRNA target and 200 nM crRNA in nuclease assay buffer (40 mM Tris-HCl, 60 mM NaCl, 6 mM MgCl2, pH 7.5) supplemented with 4U of RNase Inhibitor, Murine (M0314S, New England Biolabs). For pre-crRNA processing reactions, crRNA was omitted and pre-crRNA was used in place of labeled ssRNA target. Reactions were incubated for 1 hour at 37° C. (unless otherwise indicated) and then quenched with addition of proteinase K, EDTA, and urea (final concentrations 1 mg/mL proteinase K, 6 mM EDTA, and 400 uM Urea) for 15 minutes at 50° C. To prepare for gel electrophoresis, reactions were denatured with 4.5M urea denaturing buffer at 95° C. for 5 minutes and loaded onto a 10% Novex PAGE TBE-Urea gel (EC6885BOX, Invitrogen), which was run at 235V for 25 minutes at 60° C. Gels were imaged using an Odyssey scanner (LI-COR Biosciences).

### Example 34

### Experimental and Computational Analysis of in vitro PFS Screens

In vitro PFS screens were reverse transcribed with a target specific primer using the qScript cDNA SuperMix (95048-025, Quanta Bio) and, the target PFS region was PCR amplified and sequenced using a MiSeq (Illumina) with a single-end 150 cycle kit. PFS regions were computationally extracted and collapsed to 5 nt to have broader coverage, and collapsed PFS were counted and normalized to total reads for each sample. For a given PFS region, enrichment was measured as the log ratio compared to non-targeting control, with a 0.01 pseudocount adjustment. PFSs above a 5 depletion threshold that were enriched in both experimental replicates were collected and used to generate sequence logos

### Example 35

### In Vitro Collateral Activity Assays

In vitro collateral activity assays were performed with 233 nM purified Cas7-11 or LwaCas13a, 10 nM of unlabeled ssRNA target and 200 nM crRNA in nuclease assay buffer (40 mM Tris-HCl, 60 mM NaCl, 6 mM MgCl2, pH 7.5) supplemented with 4U of RNase Inhibitor, Murine (M0314S, New England Biolabs) and 250 nM final concentration RnaseAlert V2 (4479768, Thermo Fisher Scientific). Reactions were incubated at 37° C. and activity was read continuously in the FAM channel of a Synergy Neo2 plate reader (BioTek).

### Example 36

### Electrophoretic Mobility Shift Assay (EMSA)

EMSA reactions were performed with dilutions of Cas7-11-crRNA complex with 10 nM of labeled ssRNA target in EMSA buffer (5 mM EDTA, 2.5% glycerol, 20 mM Tris, 5 μg/mL heparin, pH 7.5) supplemented with 4U of RNase Inhibitor, Murine (M0314S, New England Biolabs). Binding occurred by incubation at 37° C. for 20 minutes, after which reactions were mixed with 2× loading buffer (0.5× TBE buffer, 10% Ficoll, 18 mM EDTA) and run on a 6% Novex PAGE TBE gel (EC6265BOX, Thermo Fisher Scientific) at 180V for 30 min at 4° C. Gels were imaged using an Odyssey scanner (LI-COR Biosciences).

### Example 37

### Next-Generation Sequencing of in Vitro Cleaved RNA

In vitro nuclease assays were performed and samples were quenched as described above using unlabeled ssRNA targets. After quenching, samples were treated with alkaline phosphatase (EF0651, Thermo Fisher Scientific) and purified with RNA Clean and Concentrator columns (R1017, Zymo Research). Libraries were then treated with the same small RNA sequencing pipeline applied to in vivo crRNA species, with the RiboMinus depletion step omitted.

### Example 38

### Design and Cloning of Mammalian Constructs

To generate vectors for testing DisCas7-11a in mammalian cells, a mammalian codon optimized DisCas7-11a sequence was ordered from Twist Biosciences, PCR amplified, and cloned into a mammalian expression vector containing combinations of modifications, including with and without NLS tags or N- or C-terminal msfGFP fusions, all under the control of a CMV promoter.

The dual luciferase reporter was cloned by PCR amplifying Gaussia and Cypridinia luciferase coding DNA, the EF1alpha and CMV promoters and assembled using the NEB Gibson Assembly (E2611S, New England Biolabs).

For mammalian DisCas7-11a guide expression, we synthesized both the full-length and mature DR sequences and cloned with golden-gate acceptor sites under U6 expression via restriction digest cloning. Individual guides were then cloned into the corresponding expression backbones for DisCas7-11a by golden-gate cloning. All mammalian DisCas7-11 plasmids are listed in Table 7. All DisCas7-11a guide sequences for knockdown or RNA editing experiments are listed in Table 8.

### Example 39

### Mammalian Cell Culture

All mammalian experiments were performed using the HEK293 FT cell line (American Type Culture Collection (ATCC)). HEK293 FT cells were grown in Dulbecco's Modified Eagle Medium with high glucose, sodium pyruvate, and GlutaMAX (Thermo Fisher Scientific), additionally supplemented with 1× penicillin-streptomycin (Thermo Fisher Scientific) and 10% fetal bovine serum (VWR Seradigm). We maintained cells below a confluency of 80%.

Transfections were performed using Lipofectamine 2000 (Thermo Fisher Scientific) in 96-well plates coated with poly-D-lysine (BD Biocoat). For transfections, cells were plated 16 hours prior to transfection at seeding densities of ˜20,000-30,000 cells per well, allowing cells to reach 90% confluency by transfection. For each well on the plate, transfection plasmids were combined with Opti-MEM I Reduced Serum Medium (Thermo Fisher) to a total of 25 μl. Separately, 24.5 μl of Opti-MEM was combined with 0.5 μl of Lipofectamine 2000. Plasmid and Lipofectamine solutions were then combined and incubated for 5-10 minutes, after which they were pipetted onto cells.

### Example 40

### Mammalian Cell RNA Knockdown Assays

To assess RNA knockdown in mammalian cells with reporter constructs, 100 ng of DisCas7-11a expression vector was co-transfected with 150 ng of guide expression plasmid and 40 ng of the knockdown reporter construct. After 48 hours, we harvested media containing secreted luciferase and measured luciferase activity using the Gaussia Luciferase Assay reagent (GAR-2B) (Targeting Systems) and Cypridina (Vargula) luciferase assay reagent (VLAR-2) (Targeting Systems) kits. Assays were performed in white 96 well plates on a plate reader (Biotek Synergy Neo 2) with an injection protocol. All replicates performed are biological replicates. Luciferase measurements were normalized by dividing the Gluc values by the Cluc values, thus normalizing for variation from well to well.

For targeting of endogenous genes, 100 ng of DisCas7-11a expression vector was co-transfected with 150 ng of guide expression plasmid. After 48 hours, we lysed cells and harvested RNA using a method previously described (Joung et al. 2017) with a gene-specific reverse transcription primers. Using Fast Advanced Master Mix (Thermo Fisher Scientific), we measured gene expression using the cDNA via qPCR and TaqMan qPCR probes for the KRAS, PPIB, CXCR4, and MALAT1 transcripts (Thermo Fisher Scientific) as well as the GAPDH control probe (Thermo Fisher Scientific). qPCR reactions were read out on a Bio-Rad CFX384 Touch Real-Time PCR Detection System, with four 5 μl technical replicates in 384-well format.

### Example 41

### RNA Editing in Mammalian Cells

To assess dDisCas7-11a-ADAR2dd editing activity in mammalian cells, we transfected 100 ng of dDisCas7-11a-ADAR2dd expression vector, 150 ng of guide expression plasmid, and 25 ng of the RNA editing reporter. After 48 hours, we harvested media from the cells, which should contain the secreted Gluc (normalization control) and Cluc (protein targeted for correction of W85X) proteins. If planning to sequence the RNA for the precise RNA editing levels, we also harvested RNA using a method previously described (Joung et al. 2017) with a gene-specific reverse transcription primer.

For luciferase measurements, we used undiluted media and the Gaussia Luciferase Assay reagent (GAR-2B) (Targeting Systems) and Cypridina (Vargula) luciferase assay reagent (VLAR-2) (Targeting Systems) kits. Assays were performed in white 96 well plates on a plate reader (Biotek Synergy Neo 2) with an injection protocol. All replicates performed are biological replicates. Luciferase measurements were normalized by dividing the Cluc values by the Gluc values, thus normalizing for variation from well to well. This ratio measurement could further be normalized by dividing by the non-targeting guide ratios.

For sequencing of RNA editing rates, we performed two rounds of PCR (NEBNext High-Fidelity 2×PCR Master Mix from New England Biolabs) using the extracted cDNA to add Illumina adaptors and sample barcodes. Illumina next generating sequencing was used to sequence editing rates in our prepared libraries on a MiSeq instrument. RNA editing rates were evaluated at the targeted adenosine in the W85X pre-termination codon.

### Example 42

### DisCas7-11-Mediated RNA-Guided RNA Interference Against MS2 Phage and RNA Knockdown in E. coli

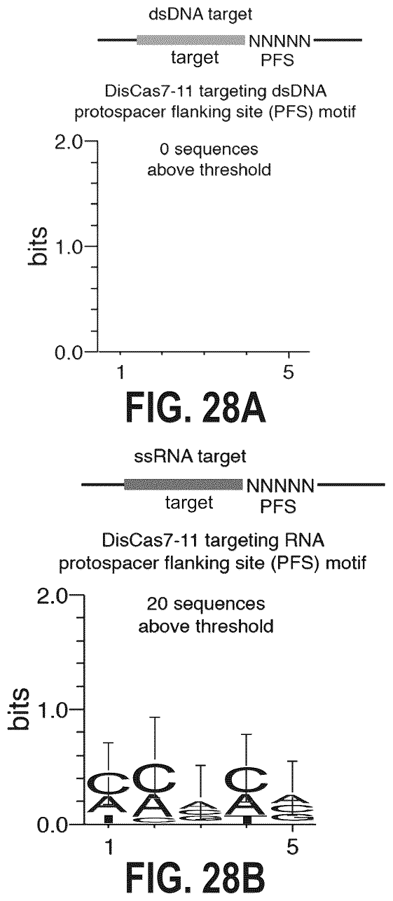

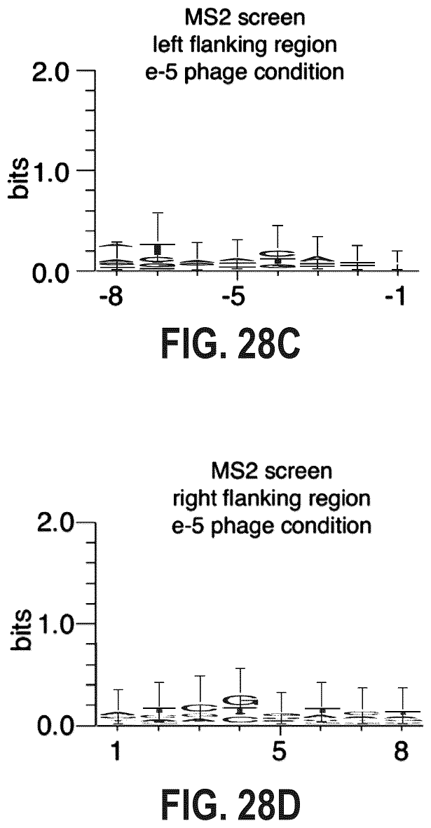

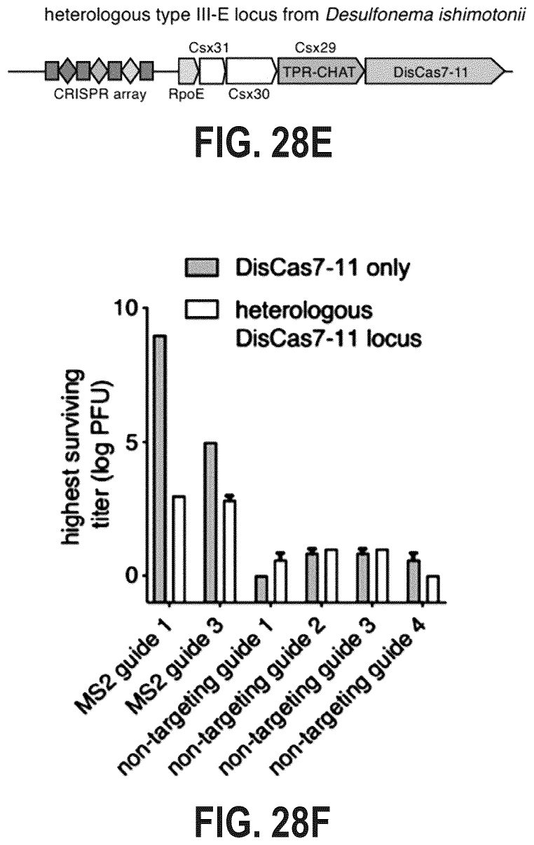

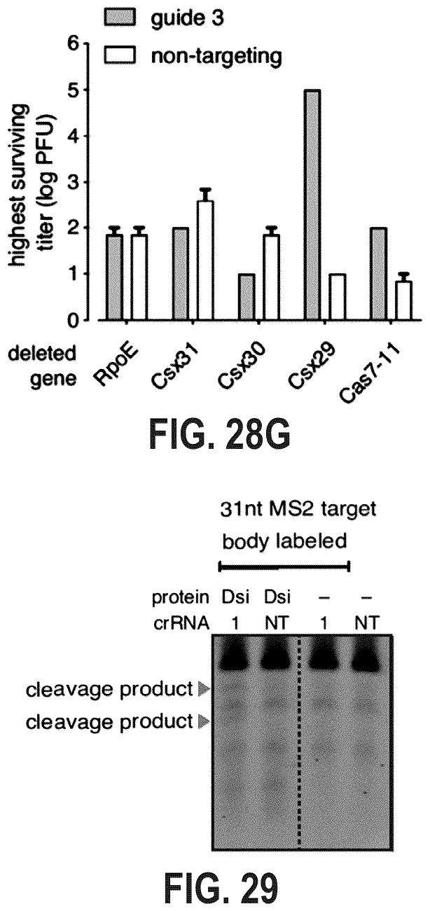

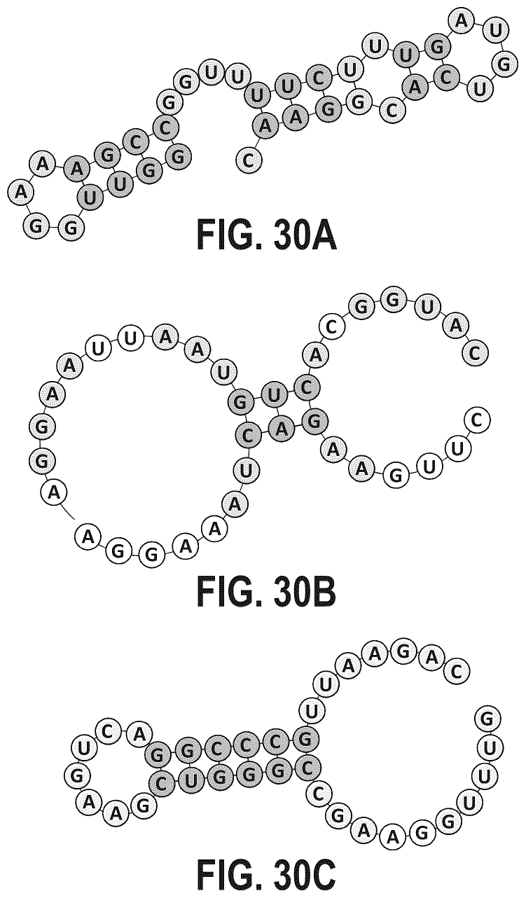

DisCas7-11-mediated RNA-guided RNA interference against MS2 phage and RNA knockdown in E. coli was investigated. The potential role of PFS in the function of the III-E systems was assessed by performing randomized PFS screens on both a DNA-only target and an expressed target at the 5′ end of an ampicillin gene. It was found that there was no signal on the DNA-only target on either side of the protospacer (FIG. 28A) and only a weak sequence preference when cleaving the expressed target (FIG. 28B). Furthermore, it was found that there were no observable trends in the flanking sequences of enriched MS2 spacers (FIGS. 28C and 28D), suggesting a lack of targeting constraints.

### Example 43

### Regulation of DisCas7-11 Interference Activity by Accessory Proteins

The regulation of DisCas7-11 interference activity by accessory proteins was assessed. Whether accessory proteins in the D. ishimotonii subtype III-E CRISPR locus could modulate RNA interference activity was investigated (FIG. 28E). Expression of the full D. ishimotonii CRISPR locus resulted in reduced MS2 interference compared to DisCas7-11 alone (FIG. 28F), prompting us to test the deletion of each putative accessory gene. Deletion of Csx29 was found to restore the interference activity. (FIG. 28G).

### Example 44

### Mapping DisCas7-11a Cleavage Mechanism

The mapping of the DisCas7-11a cleavage mechanism via in vitro cleavage of ssRNA of a 31 nt target was investigated. The DisCas7-11a cleavage of synthetic 31 nt MS2 ssRNA with a 31 nt crRNA completely duplexed to the target shows two cleavage fragments that are generated in the targeting condition when protein is generated (indicated by triangles) (FIG. 29). Other bands present in all conditions indicate incomplete products generated when in vitro transcribing the 31 nt target.

### Example 45

### DisCas7-11a-Mediated Knockdown of mRNA in Mammalian Cells

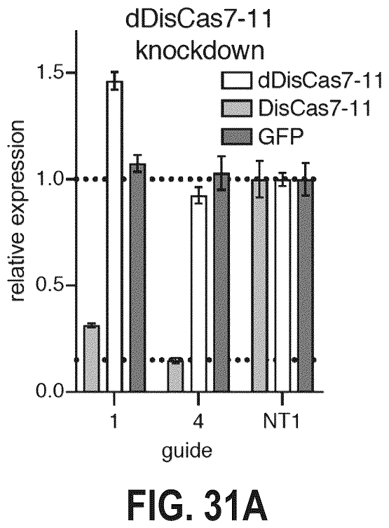

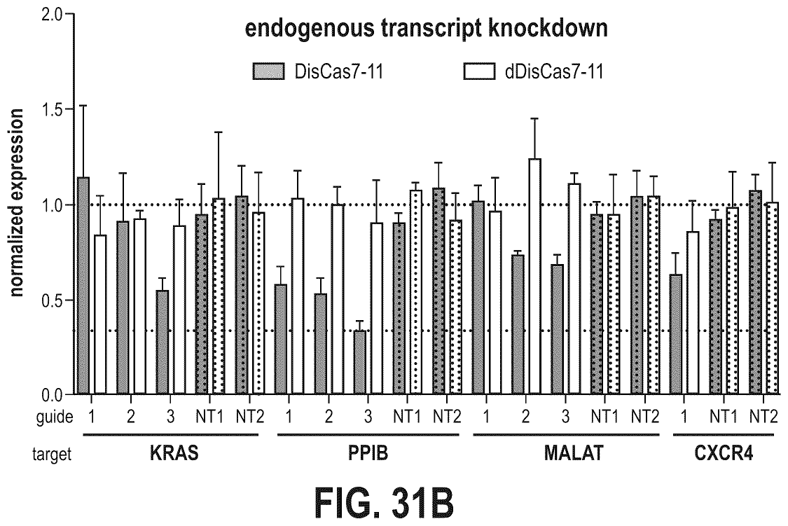

The effects of DisCas7-11a-mediated knockdown of mRNA in mammalian cells by guide variants or inactivating mutations was measured. The knockdown of Gaussia luciferase (Gluc) mRNA in mammalian cells by DisCas7-11a unmodified or with N-/C-terminal msfGFP fusions, with guides containing the full DR sequence was assessed. The guides are designed to be tiled across the Gluc transcript. The knockdown of Gluc mRNA in mammalian cells by DisCas7-11a unmodified or with N-/C-terminal msfGFP fusion, with guides containing the mature DR sequence was assessed. Guides are designed to be tiled across the Gluc transcript. The comparison of knockdown activity of Gluc mRNA in mammalian cells between active DisCas7-11a, catalytically inactive D429A/D654A DisCas7-11a, and GFP is illustrated in FIG. 31A. Guides are the same as in a and b above and have the full DR sequence. The comparison of knockdown activity of endogenous mRNA in mammalian cells between active DisCas7-11a, catalytically inactive D429A/D654A DisCas7-11a, and GFP is illustrated in FIG. 31B. Here the guides have the full DR sequence.

### Example 46

### RNA Editing Efficiency with DisCas7-11a-NES-ADAR2 Constructs

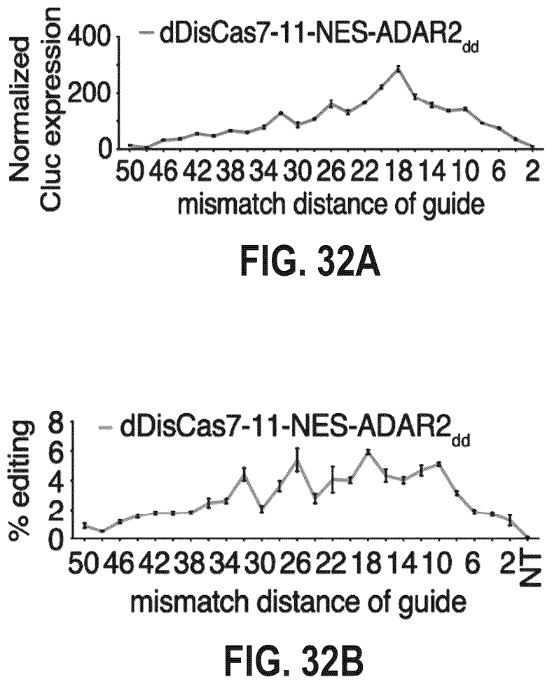

The RNA editing efficiency with active and inactive DisCas7-11a-NES-ADAR2 constructs was measured. The RNA A-to-I editing of Cypridinia luciferase (cluc) mRNA W85X mutation in mammalian cells by active DisCas7-11a-NES-ADAR2 or dead DisCas7-11a-NES-ADAR2 is shown in FIG. 32A. The guides are designed with mismatch distances between 2-50 nt. Editing is measured by restoration of Cluc luciferase activity and is normalized to the non-targeting guide condition. The RNA A-to-I editing of Cypridinia luciferase (cluc) mRNA W85X mutation in mammalian cells by active DisCas7-11a-NES-ADAR2 or dead DisCas7-11a-NES-ADAR2 is shown in FIG. 32B. The guides are designed with mismatch distances between 2-50 nt, and the editing is measured by amplicon sequencing and measuring the percent correction of the pre-termination stop codon.

### Example 47

### RNase H Cleavage of Target Showing Body Labeling

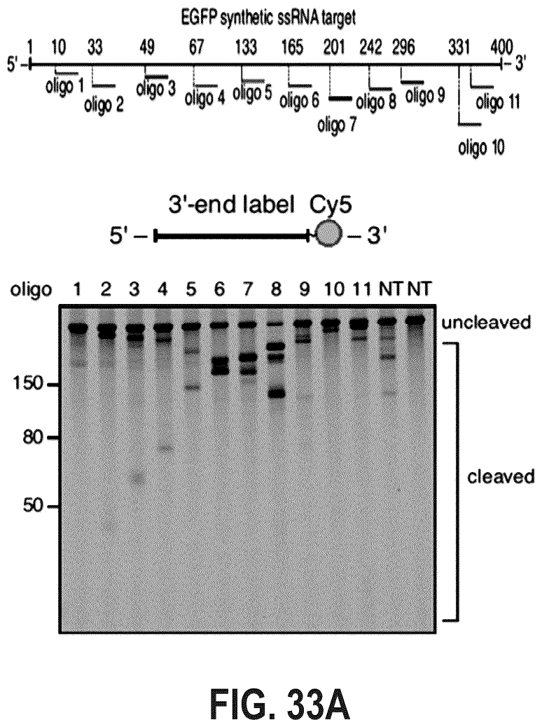

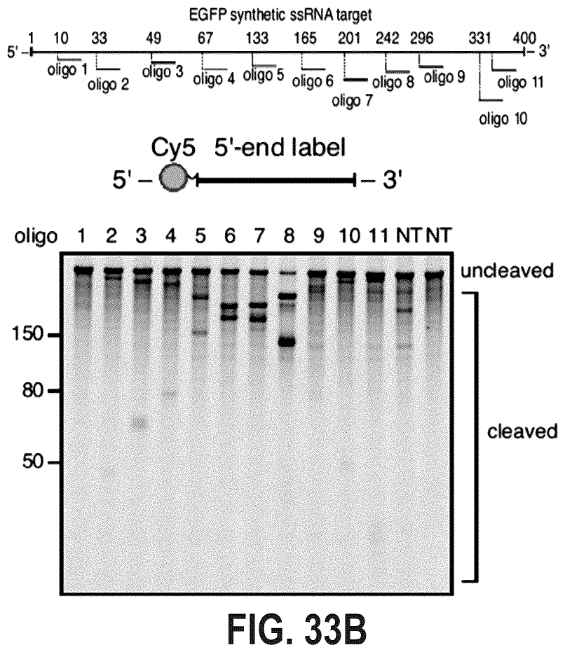

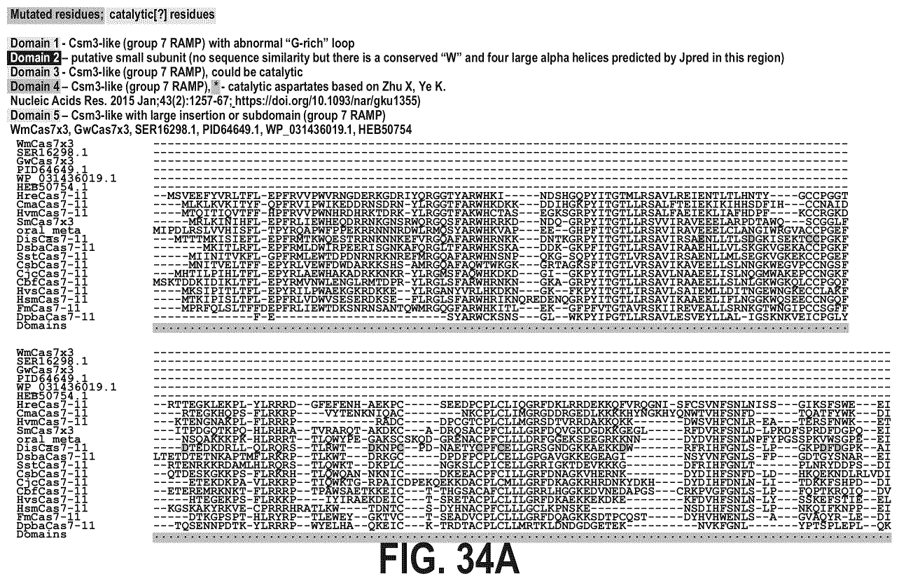

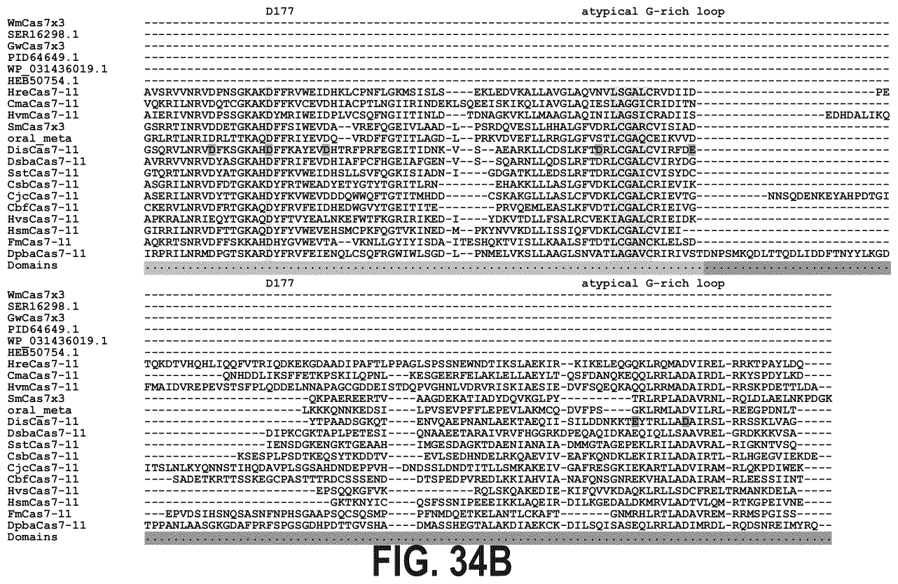

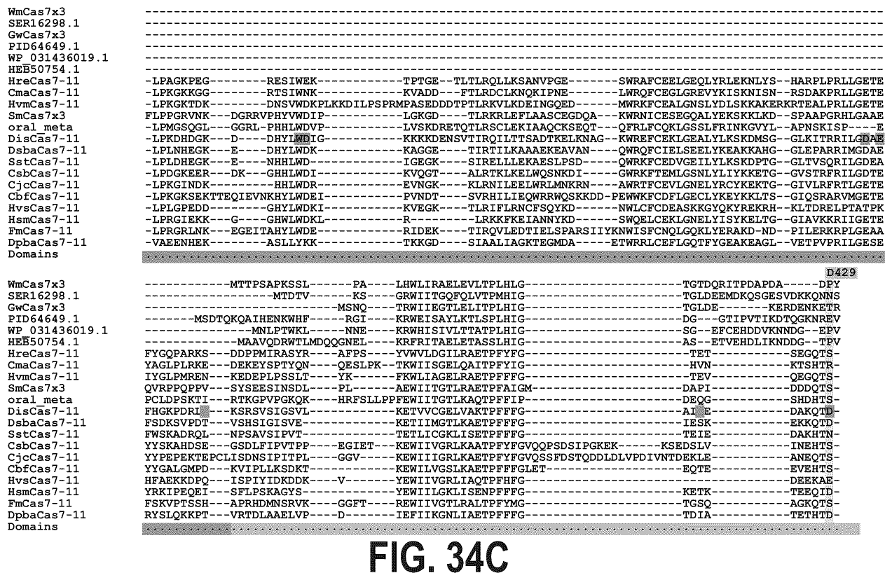

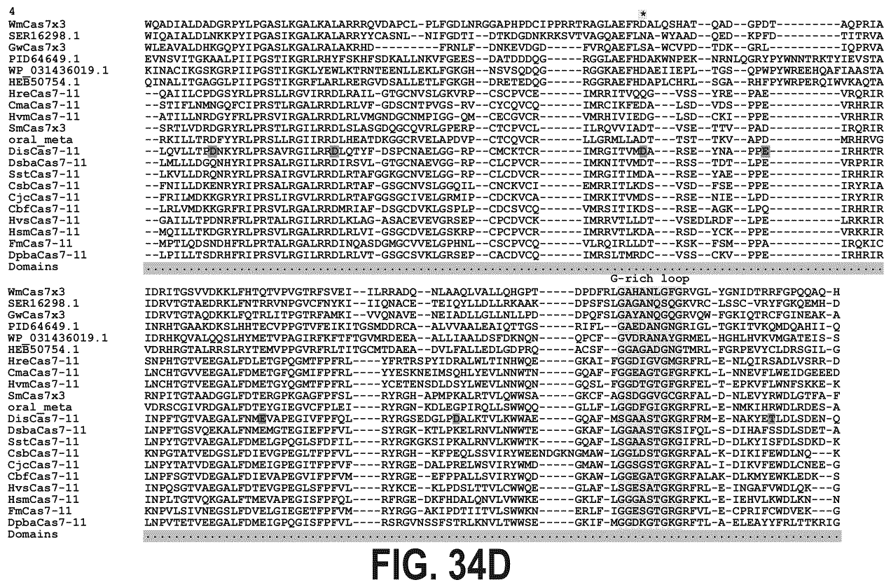

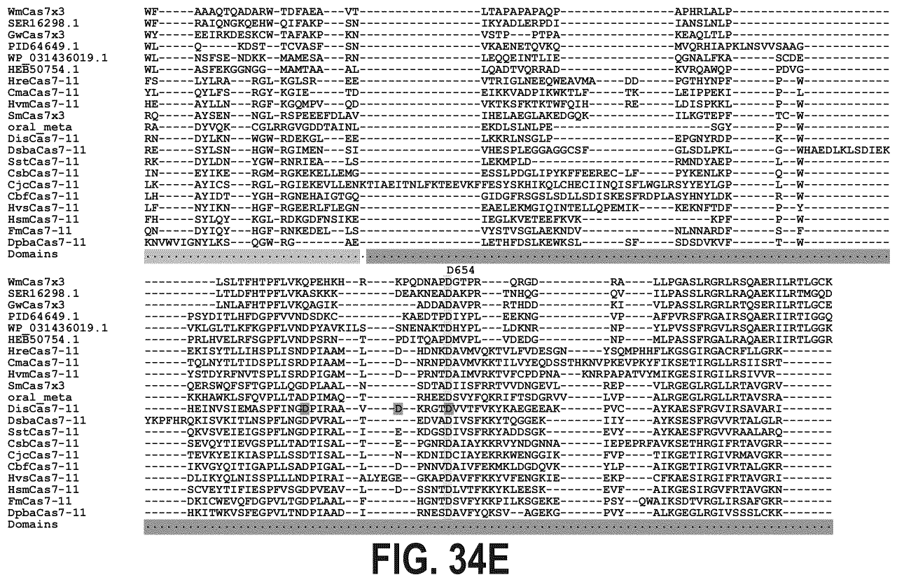

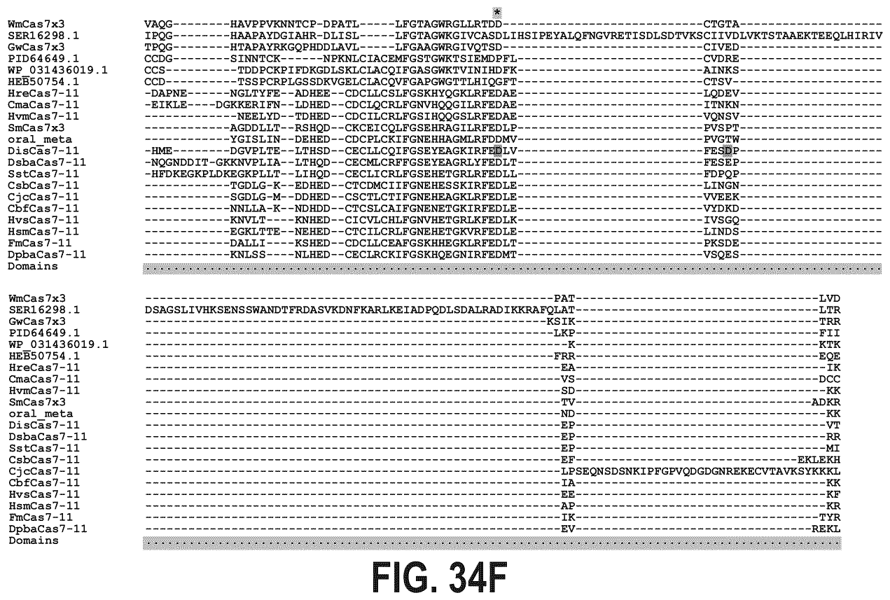

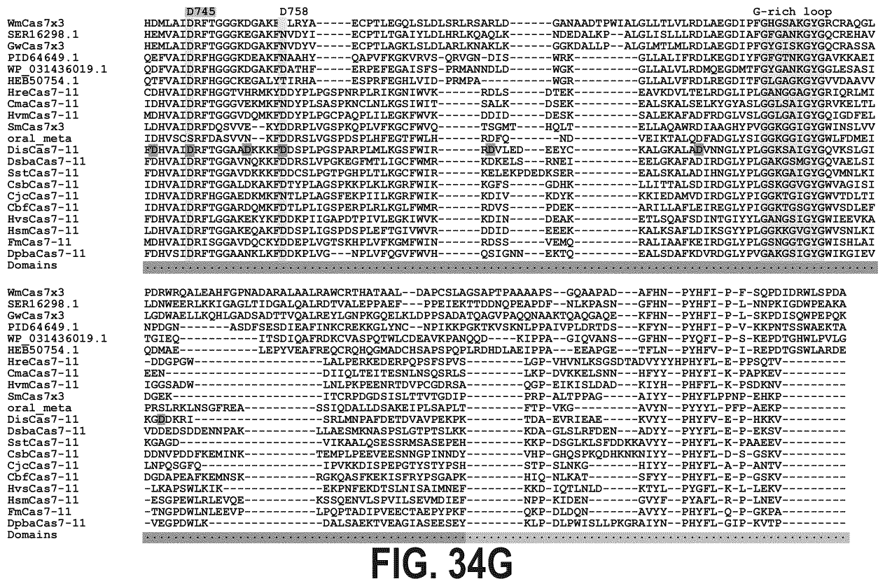

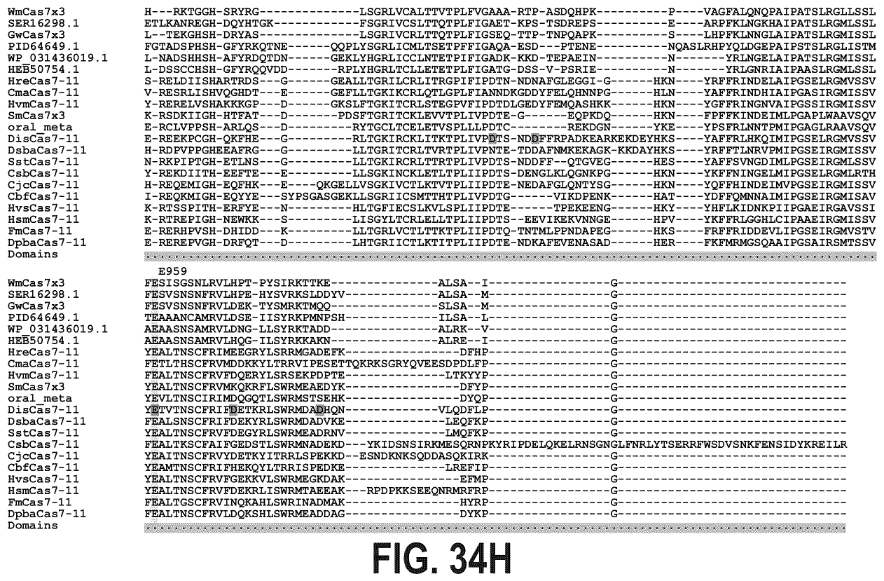

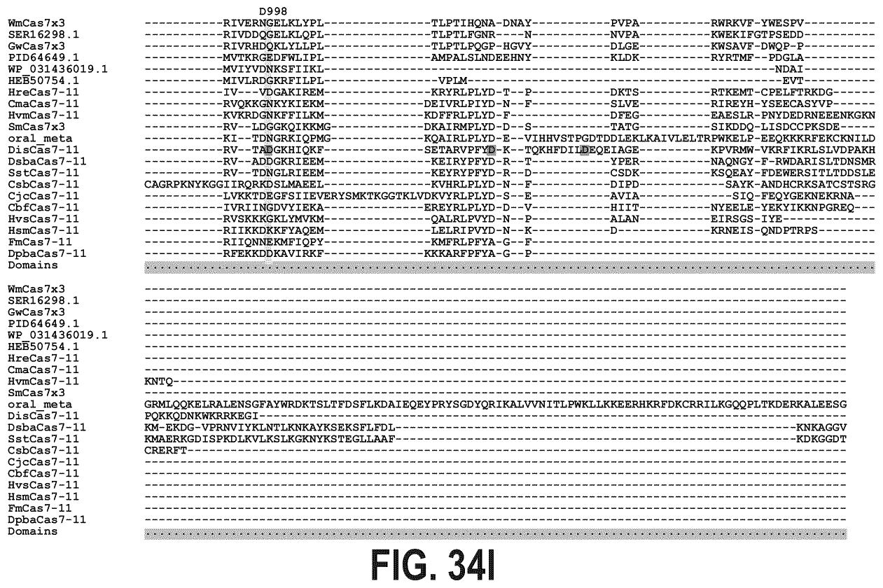

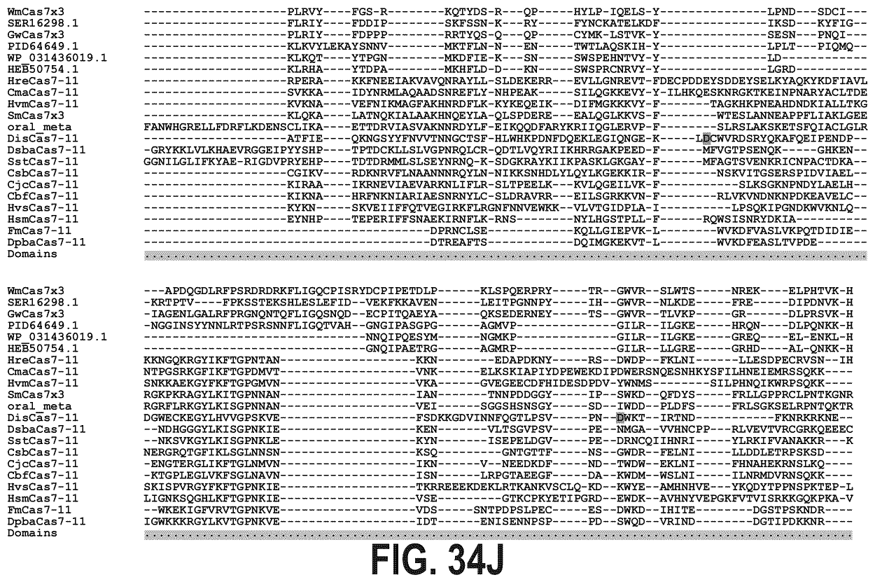

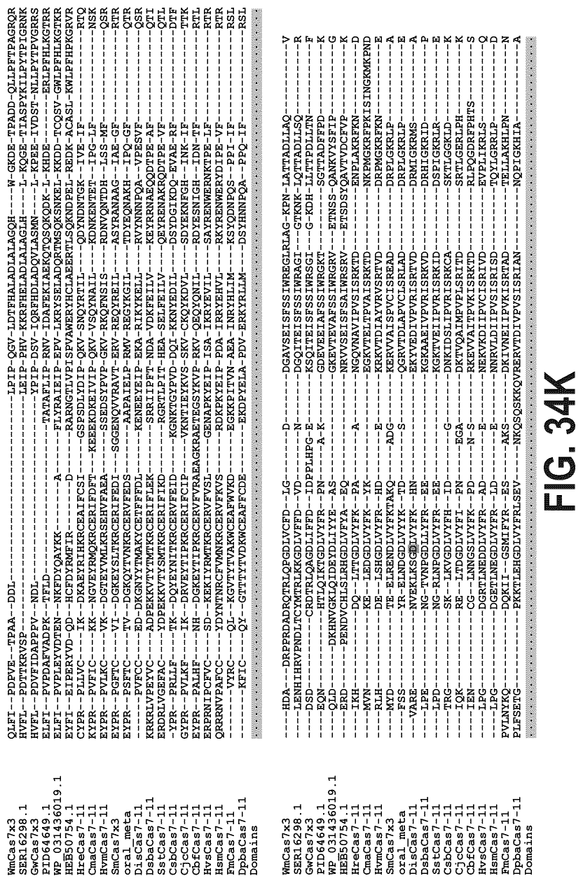

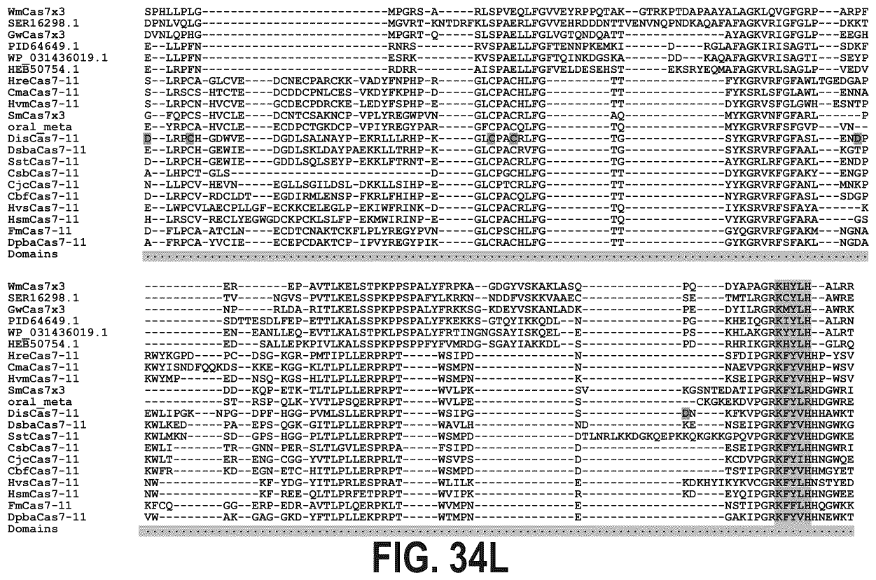

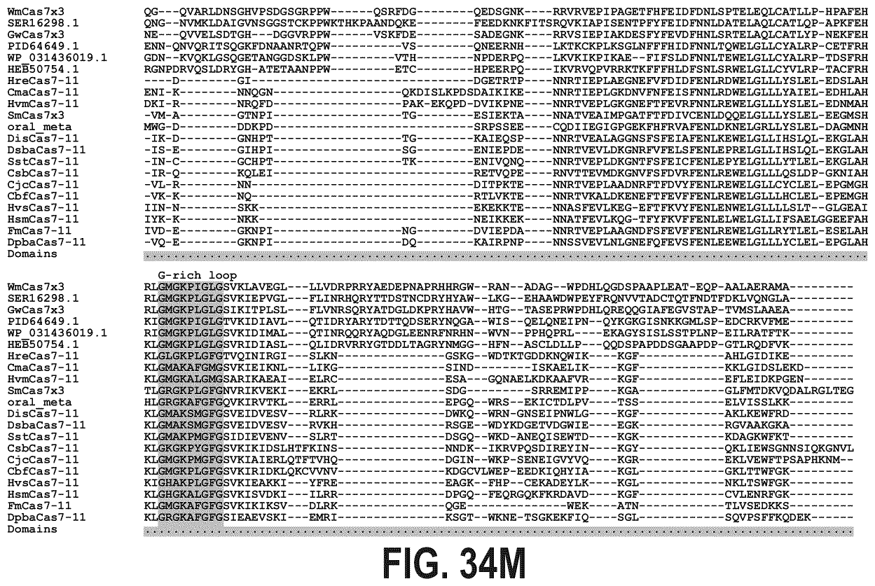

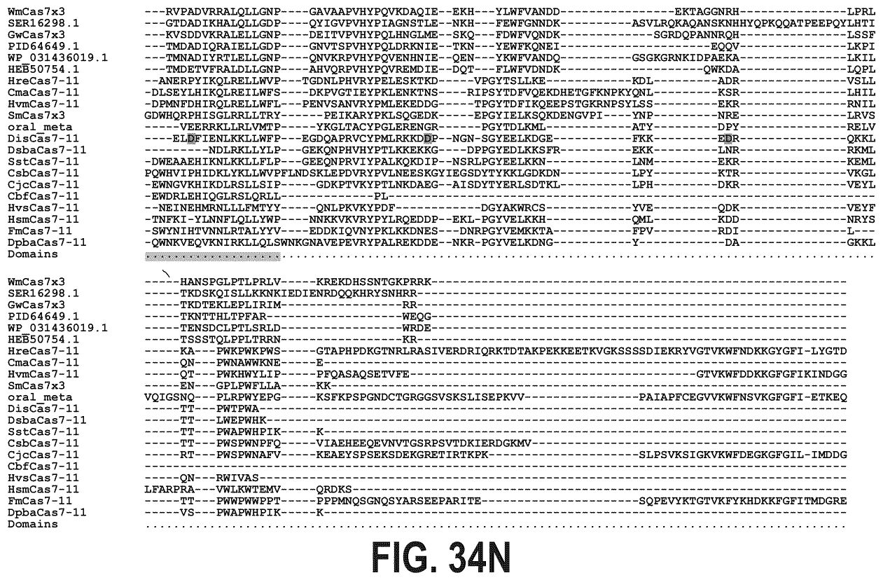

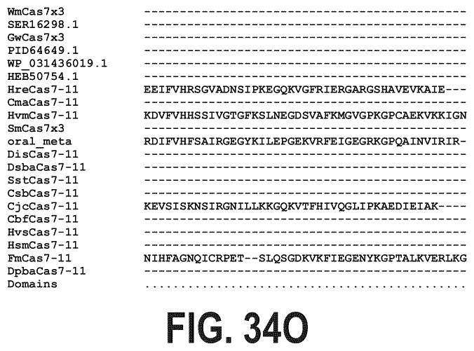

The RNase H cleavage of target showing body labeling was investigated. Cleavage of 3′ (FIG. 33A) and 5′ (FIG. 33B) labeled targeted by incubation with RNase H and indicated oligonucleotides show similar band patterns and double bands indicative of body labeling.

### Example 48

### Mechanism of the Programmable RNA Cleavage Activity of DisCas7-11

The mechanism of the programmable RNA cleavage activity of DisCas7-11 was assessed. The type III-E system from D. ishimotonii and corresponding effector DisCas7-11 were chosen, as a representative of type III-E systems to characterize in detail. The small RNA from D. ishimotonii were harvested and sequenced. Mature crRNA species indicative of pre-crRNA processing were identified (FIG. 2C), a hallmark of an active CRISPR-Cas locus. Heterologous reconstitution of the D. ishimotonii type III-E locus in E. coli demonstrated similar processing (FIG. 9A), and expression of the DisCas7-11 protein and pre-crRNA were both necessary and sufficient for mature crRNA expression (FIG. 9B and FIG. 9C), suggesting that Cas7-11 possesses pre-crRNA processing activity. Processing occurred though cleavage in the second hairpin of the direct repeat (DR) at residue 21, generating a mature rRNA with spacers of ˜18-24 nt and 14 nt DRs lacking any predicted secondary structure, similar to the mature DR tags of other type III systems (FIG. 2D). To generalize the observation of the processing activity of Cas7-11 beyond D. ishimotonii, the Candidatus Jettenia caeni type III-E locus through heterologous expression in E. coli was assessed. Robust processing of the C. Jettenia caeni pre-crRNA that resulted in mature crRNAs 37 nt in length was observed (FIG. 20), suggesting that pre-crRNA processing activity is common among type III-E systems.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

1-32. (canceled)

33. A method of cleaving an RNA target in a cell comprising providing to the cell a composition that cleaves an RNA target comprising a guide RNA that specifically hybridizes to the RNA target, and a polypeptide comprising an amino acid sequence 85% identical to an amino acid sequence selected from the group consisting of SEQ ID NOs: 1-32 and 359.

34.-35. (canceled)

36. The method of claim 33, wherein the RNA target is an ssRNA.

37. A method of treating a genetically inherited disease in a subject in need thereof comprising administering to the subject an effective amount of a composition that cleaves an RNA target comprising a guide RNA that specifically hybridizes to the RNA target, and a polypeptide comprising an amino acid sequence 85% identical to an amino acid sequence selected from the group consisting of SEQ ID NOs: 1-32 and 359.

38. The method of claim 37, wherein the genetically inherited disease is selected from the group consisting of Meier-Gorlin syndrome; Seckel syndrome 4; Joubert syndrome 5; Leber congenital amaurosis 10; Charcot-Marie-Tooth disease, type 2; leukoencephalopathy; Usher syndrome, type 2C; spinocerebellar ataxia 28; glycogen storage disease type III; primary hyperoxaluria, type I; long QT syndrome 2; Sjögren-Larsson syndrome; hereditary fructosuria; neuroblastoma; amyotrophic lateral sclerosis type 9; Kallmann syndrome 1; limb-girdle muscular dystrophy, type 2L; familial adenomatous polyposis 1; familial type 3 hyperlipoproteinemia; Alzheimer's disease, type 1; metachromatic leukodystrophy; and cancer.

39.-42. (canceled)

43. A method of modulating translation in a cell comprising administering to the cell an effective amount of a composition that cleaves an RNA target comprising a guide RNA that specifically hybridizes to the RNA target, and a polypeptide comprising an amino acid sequence 85% identical to an amino acid sequence selected from the group consisting of SEQ ID NOs: 1-32 and 359.

44.-45. (canceled)

46. The composition of claim 33, wherein the amino acid sequence is SEQ ID NO: 1.

47. The composition of claim 33, wherein the polypeptide comprises a glutamate at a position corresponding to position 70 of SEQ ID NO:1; aspartate at a position corresponding to position 429 of SEQ ID NO:1; aspartate at a position corresponding to position 487 of SEQ ID NO:1; and/or an aspartate at a position corresponding to position 654 of SEQ ID NO:1.

48. The composition of claim 33, wherein the guide RNA has a sequence with a length of from about 20 to about 53 nucleotides (nt),

49. The composition of claim 33, wherein the guide RNA is a pre-crRNA or a mature crRNA.

50. The composition of claim 33, wherein the RNA target is in a prokaryotic cell or a eukaryotic cell.

51. The composition of claim 33, wherein the polypeptide further comprises a deaminase domain for programmable RNA base editing.

52. The composition of claim 51, wherein the deaminase domain comprises an adenosine deaminase, a cytidine deaminase, or a catalytic domain thereof.

53. The composition of claim 51, wherein the deaminase domain comprises an amino acid sequence 85% identical to an amino acid sequence selected from the group consisting of SEQ ID NOs: 25-42.

54. The composition of claim 33, further comprising a vector.

55. The composition of claim 33, further comprising a cell.


SEQUENCE LISTING (647 sequences)

SEQ ID NO: 1 - protein, 1812 aa, unidentified
  REGION 1..1812: note = source = /note="Description of Unknown: Candidatus Jettenia caeni sequence"
MHTILPIHLTFLEPYRLAEWHAKADRKKNKRYLRGMSFAQWHKDKDGIGKPYITGTLLRSAVLNAAEELISLNQGMWAKEPCCNGKFETEKDKPAVLRKRPTIQWKTGRPAICDPEKQEKKDACPLCMLLGRFDKAGKRHRDNKYDKHDYDIHFDNLNLITDKKFSHPDDIASERILNRVDYTTGKAHDYFKVWEVDDDQWWQFTGTITMHDDCSKAKGLLLASLCFVDKLCGALCRIEVTGNNSQDENKEYAHPDTGIITSLNLKYQNNSTIHQDAVPLSGSAHDNDEPPVHDNDSSLDNDTITLLSMKAKEIVGAFRESGKIEKARTLADVIRAMRLQKPDIWEKLPKGINDKHHLWDREVNGKKLRNILEELWRLMNKRNAWRTFCEVLGNELYRCYKEKTGGIVLRFRTLGETEYYPEPEKTEPCLISDNSIPITPLGGVKEWIIIGRLKAETPFYFGVQSSFDSTQDDLDLVPDIVNTDEKLEANEQTSFRILMDKKGRYRIPRSLIRGVLRRDLRTAFGGSGCIVELGRMIPCDCKVCAIMRKITVMDSRSENIELPDIRYRIRLNPYTATVDEGALFDMEIGPEGITFPFVFRYRGEDALPRELWSVIRYWMDGMAWLGGSGSTGKGRFALIDIKVFEWDLCNEEGLKAYICSRGLRGIEKEVLLENKTIAEITNLFKTEEVKFFESYSKHIKQLCHECIINQISFLWGLRSYYEYLGPLWTEVKYEIKIASPLLSSDTISALLNKDNIDCIAYEKRKWENGGIKFVPTIKGETIRGIVRMAVGKRSGDLGMDDHEDCSCTLCTIFGNEHEAGKLRFEDLEVVEEKLPSEQNSDSNKIPFGPVQDGDGNREKECVTAVKSYKKKLIDHVAIDRFHGGAEDKMKFNTLPLAGSFEKPIILKGRFWIKKDIVKDYKKKIEDAMVDIRDGLYPIGGKTGIGYGWVTDLTILNPQSGFQIPVKKDISPEPGTYSTYPSHSTPSLNKGHIYYPHYFLAPANTVHREQEMIGHEQFHKEQKGELLVSGKIVCTLKTVTPLIIPDTENEDAFGLQNTYSGHKNYQFFHINDEIMVPGSEIRGMISSVYEAITNSCFRVYDETKYITRRLSPEKKDESNDKNKSQDDASQKIRKGLVKKTDEGFSIIEVERYSMKTKGGTKLVDKVYRLPLYDSEAVIASIQFEQYGEKNEKRNAKIRAAIKRNEVIAEVARKNLIFLRSLTPEELKKVLQGEILVKFSLKSGKNPNDYLAELHENGTERGLIKFTGLNMVNIKNVNEEDKDFNDTWDWEKLNIFHNAHEKRNSLKQGYPRPVLKFIKDRVEYTIPKRCERIFCIPVKNTIEYKVSSKVCKQYKDVLSDYEKNFGHINKIFTTKIQKRELTDGDLVYFIPNEGADKTVQAIMPVPLSRITDSRTLGERLPHKNLLPCVHEVNEGLLSGILDSLDKKLLSIHPEGLCPTCRLFGTTYYKGRVRFGFANLMNKPKWLTERENGCGGYVTLPLLERPRLTWSVPSDKCDVPGRKFYIHHNGWQEVLRNNDITPKTENNRTVEPLAADNRFTFDVYFENLREWELGLLCYCLELEPGMGHKLGMGKPMGFGSVKIAIERLQTFTVHQDGINWKPSENEIGVYVQKGREKLVEWFTPSAPHKNMEWNGVKHIKDLRSLLSIPGDKPTVKYPTLNKDAEGAISDYTYERLSDTKLLPHDKRVEYLRTPWSPWNAFVKEAEYSPSEKSDEKGRETIRTKPKSLPSVKSIGKVKWFDEGKGFGILIMDDGKEVSISKNSIRGNILLKKGQKVTFHIVQGLIPKAEDIEIAK

SEQ ID NO: 2 - protein, 1717 aa, unidentified
  REGION 1..1717: note = source = /note="Description of Unknown: Candidatus Scalindua brodae sequence"
MNITVELTFFEPYRLVEWFDWDARKKSHSAMRGQAFAQWTWKGKGRTAGKSFITGTLVRSAVIKAVEELLSLNNGKWEGVPCCNGSFQTDESKGKKPSFLRKRHTLQWQANNKNICDKEEACPFCILLGRFDNAGKVHERNKDYDIHFSNFDLDHKQEKNDLRLVDIASGRILNRVDFDTGKAKDYFRTWEADYETYGTYTGRITLRNEHAKKLLLASLGFVDKLCGALCRIEVIKKSESPLPSDTKEQSYTKDDTVEVLSEDHNDELRKQAEVIVEAFKQNDKLEKIRILADAIRTLRLHGEGVIEKDELPDGKEERDKGHHLWDIKVQGTALRTKLKELWQSNKDIGWRKFTEMLGSNLYLIYKKETGGVSTRFRILGDTEYYSKAHDSEGSDLFIPVTPPEGIETKEWIIVGRLKAATPFYFGVQQPSDSIPGKEKKSEDSLVINEHTSFNILLDKENRYRIPRSALRGALRRDLRTAFGSGCNVSLGGQILCNCKVCIEMRRITLKDSVSDFSEPPEIRYRIAKNPGTATVEDGSLFDIEVGPEGLTFPFVLRYRGHKFPEQLSSVIRYWEENDGKNGMAWLGGLDSTGKGRFALKDIKIFEWDLNQKINEYIKERGMRGKEKELLEMGESSLPDGLIPYKFFEERECLFPYKENLKPQWSEVQYTIEVGSPLLTADTISALTEPGNRDAIAYKKRVYNDGNNAIEPEPRFAVKSETHRGIFRTAVGRRTGDLGKEDHEDCTCDMCIIFGNEHESSKIRFEDLELINGNEFEKLEKHIDHVAIDRFTGGALDKAKFDTYPLAGSPKKPLKLKGRFWIKKGFSGDHKLLITTALSDIRDGLYPLGSKGGVGYGWVAGISIDDNVPDDFKEMINKTEMPLPEEVEESNNGPINNDYVHPGHQSPKQDHKNKNIYYPHYFLDSGSKVYREKDIITHEEFTEELLSGKINCKLETLTPLIIPDTSDENGLKLQGNKPGHKNYKFFNINGELMIPGSELRGMLRTHFEALTKSCFAIFGEDSTLSWRMNADEKDYKIDSNSIRKMESQRNPKYRIPDELQKELRNSGNGLFNRLYTSERRFWSDVSNKFENSIDYKREILRCAGRPKNYKGGIIRQRKDSLMAEELKVHRLPLYDNFDIPDSAYKANDHCRKSATCSTSRGCRERFTCGIKVRDKNRVFLNAANNNRQYLNNIKKSNHDLYLQYLKGEKKIRFNSKVITGSERSPIDVIAELNERGRQTGFIKLSGLNNSNKSQGNTGTTFNSGWDRFELNILLDDLETRPSKSDYPRPRLLFTKDQYEYNITKRCERVFEIDKGNKTGYPVDDQIKKNYEDILDSYDGIKDQEVAERFDTFTRGSKLKVGDLVYFHIDGDNKIDSLIPVRISRKCASKTLGGKLDKALHPCTGLSDGLCPGCHLFGTTDYKGRVKFGFAKYENGPEWLITRGNNPERSLTLGVLESPRPAFSIPDDESEIPGRKFYLHHNGWRIIRQKQLEIRETVQPERNVTTEVMDKGNVFSFDVRFENLREWELGLLLQSLDPGKNIAHKLGKGKPYGFGSVKIKIDSLHTFKINSNNDKIKRVPQSDIREYINKGYQKLIEWSGNNSIQKGNVLPQWHVIPHIDKLYKLLWVPFLNDSKLEPDVRYPVLNEESKGYIEGSDYTYKKLGDKDNLPYKTRVKGLTTPWSPWNPFQVIAEHEEQEVNVTGSRPSVTDKIERDGKMV

SEQ ID NO: 3 - protein, 1659 aa, unidentified
  REGION 1..1659: note = source = /note="Description of Unknown: Desulfobacteraceae bacterium 4572_88 sequence"
MKITLRFLEPFRMLDWIRPEERISGNKAFQRGLTFARWHKSKADDKGKPFITGTLLRSAVIRAAEHLLVLSKGKVGEKACCPGKFLTETDTETNKAPTMFLRKRPTLKWTDRKGCDPDFPCPLCELLGPGAVGKKEGEAGINSYVNFGNLSFPGDTGYSNAREIAVRRVVNRVDYASGKAHDFFRIFEVDHIAFPCFHGEIAFGENVSSQARNLLQDSLRFTDRLCGALCVIRYDGDIPKCGKTAPLPETESIQNAAEETARAIVRVFHGGRKDPEQAQIDKAEQIQLLSAAVRELGRDKKKVSALPLNHEGKEDHYLWDKKAGGETIRTILKAAAEKEAVANQWRQFCIELSEELYKEAKKAHGGLEPARRIMGDAEFSDKSVPDTVSHSIGISVEKETIIMGTLKAETPFFFGIESKEKKQTDLMLLLDGQNHYRIPRSALRGILRRDIRSVLGTGCNAEVGGRPCLCPVCRIMKNITVMDTRSSTDTLPEVRPRIRLNPFTGSVQEKALFNMEMGTEGIEFPFVLSYRGKKTLPKELRNVLNWWTEGKAFLGGAASTGKSIFQLSDIHAFSSDLSDETARESYLSNHGWRGIMENSIVHESPLEGGAGGCSFGLSDLPKLGWHAEDLKLSDIEKYKPFHRQKISVKITLNSPFLNGDPVRALTEDVADIVSFKKYTQGGEKIIYAYKSESFRGVVRTALGLRNQGNDDITGKKNVPLIALTHQDCECMLCRFFGSEYEAGRLYFEDLTFESEPEPRRFDHVAIDRFTGGAVNQKKFDDRSLVPGKEGFMTLIGCFWMRKDKELSRNEIEELGKAFADIRDGLYPLGAKGSMGYGQVAELSIVDDEDSDDENNPAKLLAESMKNASPSLGTPTSLKKKDAGLSLRFDENADYYPYYFLEPEKSVHRDPVPPGHEEAFRGGLLTGRITCRLTVRTPLIVPNTETDDAFNMKEKAGKKKDAYHKSYRFFTLNRVPMIPGSEIRGMISSVFEALSNSCFRIFDEKYRLSWRMDADVKELEQFKPGRVADDGKRIEEMKEIRYPFYDRTYPERNAQNGYFRWDARISLTDNSMRKMEKDGVPRNVIYKLNTLKNKAYKSEKSFLFDLKNKAGGVGRYKKLVLKHAEVRGGEIPYYSHPTPTDCKLLSLVGPNRQLCRQDTLVQYRIIKHRRGAKPEEDFMFVGTPSENQKGHKENNDHGGGYLKISGPNKIEKENVLTSGVPSVPENMGAVVHNCPPRLVEVTVRCGRKQEEECKRKRLVPEYVCADPEKKVTYTMTKRCERIFLEKSRRIIPFTNDAVDKFEILVKEYRRNAEQQDTPEAFQTILPENGTVNPGDLLYFREEKGKAAEIVPVRISRKVDDRHIGKRIDPELRPCHGEWIEDGDLSKLDAYPAEKKLLTRHPKGLCPACRVFGTGSYKSRVRFGFAALKGTPKWLKEDPAEPSQGKGITLPLLERPRPTWAVLHNDKENSEIPGRKFYVHHNGWKGISEGIHPISGENIEPDENNRTVEVLDKGNRFVFELSFENLEPRELGLLIHSLQLEKGLAHKLGMAKSMGFGSVEIDVESVRVKHRSGEWDYKDGETVDGWIEEGKRGVAAKGKANDLRKLLYLPGEKQNPHVHYPTLKKEKKGDPPGYEDLKKSFREKKLNRRKMLTTLWEPWHK

SEQ ID NO: 4 - protein, 1657 aa, unidentified
  REGION 1..1657: note = source = /note="Description of Unknown: Candidatus Magnetomorum sp. HK-1 sequence"
MLKLKVKITYFQPFRVIPWIKEDDRNSDRNYLRGGTFARWHKDKKDDIHGKPYITGTLLRSALFTEIEKIKIHHSDFIHCCNAIDRTEGKHQPSFLRKRPVYTENKNIQACNKCPLCLIMGRGDDRGEDLKKKKHYNGKHYQNWTVHFSNFDTQATFYWKDIVQKRILNRVDQTCGKAKDFFKVCEVDHIACPTLNGIIRINDEKLSQEEISKIKQLIAVGLAQIESLAGGICRIDITNQNHDDLIKSFFETKPSKILQPNLKESGEERFELAKLELLAEYLTQSFDANQKEQQLRRLADAIRDLRKYSPDYLKDLPKGKKGGRTSIWNKKVADDFTLRDCLKNQKIPNELWRQFCEGLGREVYKISKNISNRSDAKPRLLGETEYAGLPLRKEDEKEYSPTYQNQESLPKTKWIISGELQAITPFYIGHVNKTSHTRSTIFLNMNGQFCIPRSTLRGALRRDLRLVFGDSCNTPVGSRVCYCQVCQIMRCIKFEDALSDVDSPPEVRHRIRLNCHTGVVEEGALFDMETGFQGMIFPFRLYYESKNEIMSQHLYEVLNNWTNGQAFFGGEAGTGFGRFKLLNNEVFLWEIDGEEEDYLQYLFSRGYKGIETDEIKKVADPIKWKTLFTKLEIPPEKIPLTQLNYTLTIDSPLISRDPIAAMLDNRNPDAVMVKKTILVYEQDSSTHKNVPKEVPKYFIKSETIRGLLRSIISRTEIKLEDGKKERIFNLDHEDCDCLQCRLFGNVHQQGILRFEDAEITNKNVSDCCIDHVAIDRFTGGGVEKMKFNDYPLSASPKNCLNLKGSIWITSALKDSEKEALSKALSELKYGYASLGGLSAIGYGRVKELTLEENDIIQLTEITESNLNSQSRLSLKPDVKKELSNNHFYYPHYFIKPAPKEVVRESRLISHVQGHDTEGEFLLTGKIKCRLQTLGPLFIANNDKGDDYFELQHNNPGHLNYAFFRINDHIAIPGASIRGMISSVFETLTHSCFRVMDDKKYLTRRVIPESETTQKRKSGRYQVEESDPDLFPGRVQKKGNKYKIEKMDEIVRLPIYDNFSLVERIREYHYSEECASYVPSVKKAIDYNRMLAQAADSNREFLYNHPEAKSILQGKKEVYYILHKQESKNRGKTKEINPNARYACLTDENTPGSRKGFIKFTGPDMVTVNKELKSKIAPIYDPEWEKDIPDWERSNQESNHKYSFILHNEIEMRSSQKKKYPRPVFICKKNGVEYRMQKRCERIFDFTKEEEKDKEIVIPQKVVSQYNAILKDNKENTETIPGLFNSKMVNKELEDGDLVYFKYKEGKVTELTPVAISRKTDNKPMGKRFPKISINGKMKPNDSLRSCSHTCTEDCDDCPNLCESVKDYFKPHPDGLCPACHLFGTTFYKSRLSFGLAWLENNAKWYISNDFQQKDSKKEKGGKLTLPLLERPRPTWSMPNNNAEVPGRKFYVHHPWSVENIKNNQGNQKDISLKPDSDAIKIKENNRTIEPLGKDNVFNFEISFNNLRDWELGLLLYAIELEDHLAHKLGMAKAFGMGSVKIEIKNLLIKGSINDISKAELIKKGFKKLGIDSLEKDDLSEYLHIKQLREILWFSDKPVGTIEYPKLENKTNSRIPSYTDFVQEKDHETGFKNPKYQNLKSRLHILQNPWNAWWKNEE

SEQ ID NO: 5 - protein, 1601 aa, Desulfonema ishimotonii
MTTTMKISIEFLEPFRMTKWQESTRRNKNNKEFVRGQAFARWHRNKKDNTKGRPYITGTLLRSAVIRSAENLLTLSDGKISEKTCCPGKFDTEDKDRLLQLRQRSTLRWTDKNPCPDNAETYCPFCELLGRSGNDGKKAEKKDWRFRIHFGNLSLPGKPDFDGPKAIGSQRVLNRVDFKSGKAHDFFKAYEVDHTRFPRFEGEITIDNKVSAEARKLLCDSLKFTDRLCGALCVIRFDEYTPAADSGKQTENVQAEPNANLAEKTAEQIISILDDNKKTEYTRLLADAIRSLRRSSKLVAGLPKDHDGKDDHYLWDIGKKKKDENSVTIRQILTTSADTKELKNAGKWREFCEKLGEALYLKSKDMSGGLKITRRILGDAEFHGKPDRLEKSRSVSIGSVLKETVVCGELVAKTPFFFGAIDEDAKQTDLQVLLTPDNKYRLPRSAVRGILRRDLQTYFDSPCNAELGGRPCMCKTCRIMRGITVMDARSEYNAPPEIRHRTRINPFTGTVAEGALFNMEVAPEGIVFPFQLRYRGSEDGLPDALKTVLKWWAEGQAFMSGAASTGKGRFRMENAKYETLDLSDENQRNDYLKNWGWRDEKGLEELKKRLNSGLPEPGNYRDPKWHEINVSIEMASPFINGDPIRAAVDKRGTDVVTFVKYKAEGEEAKPVCAYKAESFRGVIRSAVARIHMEDGVPLTELTHSDCECLLCQIFGSEYEAGKIRFEDLVFESDPEPVTFDHVAIDRFTGGAADKKKFDDSPLPGSPARPLMLKGSFWIRRDVLEDEEYCKALGKALADVNNGLYPLGGKSAIGYGQVKSLGIKGDDKRISRLMNPAFDETDVAVPEKPKTDAEVRIEAEKVYYPHYFVEPHKKVEREEKPCGHQKFHEGRLTGKIRCKLITKTPLIVPDTSNDDFFRPADKEARKEKDEYHKSYAFFRLHKQIMIPGSELRGMVSSVYETVTNSCFRIFDETKRLSWRMDADHQNVLQDFLPGRVTADGKHIQKFSETARVPFYDKTQKHFDILDEQEIAGEKPVRMWVKRFIKRLSLVDPAKHPQKKQDNKWKRRKEGIATFIEQKNGSYYFNVVTNNGCTSFHLWHKPDNFDQEKLEGIQNGEKLDCWVRDSRYQKAFQEIPENDPDGWECKEGYLHVVGPSKVEFSDKKGDVINNFQGTLPSVPNDWKTIRTNDFKNRKRKNEPVFCCEDDKGNYYTMAKYCETFFFDLKENEEYEIPEKARIKYKELLRVYNNNPQAVPESVFQSRVARENVEKLKSGDLVYFKHNEKYVEDIVPVRISRTVDDRMIGKRMSADLRPCHGDWVEDGDLSALNAYPEKRLLLRHPKGLCPACRLFGTGSYKGRVRFGFASLENDPEWLIPGKNPGDPFHGGPVMLSLLERPRPTWSIPGSDNKFKVPGRKFYVHHHAWKTIKDGNHPTTGKAIEQSPNNRTVEALAGGNSFSFEIAFENLKEWELGLLIHSLQLEKGLAHKLGMAKSMGFGSVEIDVESVRLRKDWKQWRNGNSEIPNWLGKGFAKLKEWFRDELDFIENLKKLLWFPEGDQAPRVCYPMLRKKDDPNGNSGYEELKDGEFKKEDRQKKLTTPWTPWA

SEQ ID NO: 6 - protein, 1573 aa, unidentified
  REGION 1..1573: note = source = /note="Description of Unknown: Candidatus Brocadia fulgida sequence"
MSKTDDKIDIKLTFLEPYRMVNWLENGLRMTDPRYLRGLSFARWHRNKNGKAGRPYITGTLLRSAVIRAAEELLSLNLGKWGKQLCCPGQFETEREMRKNKTFLRRRPTPAWSAETKKEICTTHGSACAFCLLLGRRLHGGKEDVNEDAPGSCRKPVGFGNLSLPFQPTKRQIQDVCKERVLNRVDFRTGKAQDYFRVFEIDHEDWGVYTGEITITEPRVQEMLEASLKFVDTLCGALCRIEIVGSADETKRTTSSKEGCPASTTTRDCSSSENDDTSPEDPVREDLKKIAHVIANAFQNSGNREKVHALADAIRAMRLEESSIINTLPKGKSEKTTEQIEVNKHYLWDEIPVNDTSVRHILIEQWRRWQSKKDDPEWWKFCDFLGECLYKEYKKLTSGIQSRARVMGETEYYGALGMPDKVIPLLKSDKTKEWILVGSLKAETPFFFGLETEQTEEVEHTSLRLVMDKKGRFRIPRSVLRGALRRDMRIAFDSGCDVKLGSPLPCDCSVCQVMRSITIKDSRSEAGKLPQIRHRIRLNPFSGTVDEGALFDIEVAPEGVIFPFVMRYRGEEFPPALLSVIRYWQDGKAWLGGEGATGKGRFALAKDLKMYEWKLEDKSLHAYIDTYGHRGNEHAIGTGQGIDGFRSGSLSDLLSDISKESFRDPLASYHNYLDKRWIKVGYQITIGAPLLSADPIGALLDPNNVDAIVFEKMKLDGDQVKYLPAIKGETIRGIVRTALGKRNNLLAKNDHDDCTCSLCAIFGNENETGKIRFEDLEVYDKDIAKKIDHVAIDRFTGGARDQMKFDTLPLIGSPERPLRLKGLFWMRRDVSPDEKARILLAFLEIREGLYPIGGKTGSGYGWVSDLEFDGDAPEAFKEMNSKRGKQASFKEKISFRYPSGAPKHIQNLKATSFYYPHYFLEPGSKVIREQKMIGHEQYYESYPSGASGEKLLSGRIICSMTTHTPLIVPDTGVIKDPENKHATYDFFQMNNAIMIPGSEIRGMISAVYEAMTNSCFRIFHEKQYLTRRISPEDKELREFIPGIVRIINGDVYIEKAEREYRLPLYDDVHIITNYEELEYEKYIKKNPGREQKIKNAHRFNKNIARIAESNRNYLCSLDRAVRREILSGRKKVNFRLVKVNDNKNPDKEAVELCKTGPLEGLVKFSGLNAVNISNLRPGTAEEGFDAKWDMWSLNIILNRMDVRNSQKKEYPRPALHFNHDGKEYTIPKRCERVFVRAEAGKRAETEGSYKVPRKVQEQYQNILRDYESNIGHIDNTFRTLIENCGLNNGSLVYFKPDNSRKEVVAITPVKISRKTDRLPQGDRFPHTSSDLRPCVRDCLDTEGDIRMLENSPFKRLFHIHPEGLCPACQLFGTTNYRGRVRFGFASLSDGPKWFRKDEGNETCHITLPLLERPRPTWSMPDDTSTIPGRKFYVHHMGYETVKKNQRTLVKTENNRTVKALDKENEFTFEVFFENLREWELGLLLHCLELEPEMGHKLGMGKPLGFGSVKIRIDKLQKCVVNVKDGCVLWEPEEDKIQHYIAKGLGKLTTWFGKEWDRLEHIQGLRSLQRLLPL

SEQ ID NO: 7 - protein, 1526 aa, unidentified
  REGION 1..1526: note = source = /note="Description of Unknown: Deltaproteobacteria bacterium sequence"
FESYARWCKSNSGLWKPYIPGTLLRSAVLESVEYLLALIGSKNKVEICPGLYTQSENNPDTKYLRRRPWYELHAQKEICKTRDTACPLCLLMRTKLDNDGDGETEKNVKFGNLYPTSPLEPLQKIRPRILNRMDPGTSKARDYFRVFEIENQLCSQFRGWIWLSGDLPNMELVKSLLAAGLSNVATLAGAVCRIRIVSTDNPSMKQDLTTQDLIDDFTNYYLKGDTPPANLAASGKGDAFPRFSPGSGDHPDTTGVSHADMASSHEGTALAKDIAEKCKDILSQISASEQLRRLADIMRDLRQDSNREIMYRQVAEENHEKASLLYKKTKKGDSIAALIAGKTEGMDAETWRRLCEFLGQTFYGEAKEAGLVETPVPRILGESERYSLQKKPTVRTDLAAELVPDIEFIIKGNLIAETPFFFGTDIATETHTDLPILLTSDRHFRIPRSVLRGILRRDLRLVTGSGCSVKLGRSEPCACDVCQIMRSLTMRDCVSSCKVPPEIRHRIRLNPVTETVEEGALFDMEIGPQGISFPFVLRSRGVNSSFSTRLKNVLTWWSEGKIFMGGDKGTGKGRFTLAELEAYYFRLTTKRIGKNVWVIGNYLKSQGWRGAELETHFDSLKEWKSLSFSDSDVKVFTWHKITWKVSFEGPVLTNDPIAADIRNESDAVFYQKSVAGEKGPVYALKGEGLRGIVSSSLCKKKNLSSNLHEDCECLRCKIFGSKHQEGNIRFEDMTVSQESEVREKLFDHVSIDRFTGGAANKLKFDDKPLVGNPLVFQGVFWVHQSIGNNEKTQEALSDAFKDVRDGLYPVGAKGSIGYGWIKGIEVVEGPDWLKDALSAEKTVEAGIASEESEYKLPDLPWISLLPKGRAIYNPHYFLGIPKVTPEREREPVGHDRFQTDLHTGRIICTLKTITPLIIPDTENDKAFEVENASADHERFKFMRMGSQAAIPGSAIRSMTSSVFEALTNSCFRVLDQKSHLSWRMEADDAGDYKPGRFEKKDDKAVIRKFKKKARFPFYAGPDTREAFTSDQIMGKEKVTLWVKDFEASLTVPDEIGWKKKRGYLKVTGPNKVEIDTENISENNPSPPDSWQDVRINDDGTIPDKKNRKFICQYGTTTYTVDKWCEAFFCDEEKDPYELAPDVERKYRLLMDSYHNNPQAPPQIFRSLPLFSETGPKKTLEHGDLVYFRLSEVNKQSQSKKQVRERVTDIVPVSISRIANNQPIGKHIAAAFRPCAYVCIEECEPCDAKTCPIPVYREGYPIKGLCRACHLFGTTGYKGRVRFSFAKLNGDAVWAKGAGGKDYFTLPLLEKPRPTWTMPNEGAKIPGRKFYVHHNEWKTVQEGKNPIDQKAIRPNPNNSSVEVLNLGNEFQFEVSFENLEEWELGLLLYCLELEPGLAHKLGRGKAFGFGSIEAEVSKIEMRIKSGTWKNETSGKEKFIQSGLSQVPSFFKQDEKQWNKVEQVKNIRKLLQLSWNKGNAVEPEVRYPALREKDDENKRPGYVELKDNGYDAGKKLVSPWAPWHPIKK

SEQ ID NO: 8 - protein, 1403 aa, unidentified
  MOD_RES 1394..1396: note = Any amino acid
  REGION 1..1403: note = source = /note="Description of Unknown: Deltaproteobacteria bacterium RIFOXYD12_FULL_50_9 sequence"
MTKKPGTEDKATLWGKESASKSVKTILEESIQGFTVEQKRSFFANLADQLVSRAGEQGAKSVRSQGLIIGRKENYAKPSAQEPTRHHLYRQPSNASAFLATGWLIAETPFFIGSGTEGQKQTDDQAESLHLRTLRDGHGRFRIPFTTIRGVMDKELRDILQAGCAKGRSLRAPCPCQVCTLMRRIQVRDAIAADILPPDLRMRTRIDPSHGTVAHLFSLEMAPQGLKLPFFLKLKGVETIDPDKELLEILNDWSAGQCFLGGLWGTGKGRFRLDDLQWHRLELDNADYYTPLLQDRFFAGETISDLRQGLQSINIQPERIPAQTPSRNMPYCRVDCILEFKSPVLSGDPVAALFESDAPDNVAYKKPVVQYDETGRLRTTDPGPVEMLTCLKGEGVRGVVAYLAGKAYDQHDLSHDSCNCTFCQAFGNGQKAGSLRFDDFMPVQFESDQAGNFSWSPHTPHAMRSDRVALDVFGGAMPEAKFDDRPLAASPGKPLNFKSTIWYREDMGKEAGKALKRALIDLQNNMAAIGSGGGIGRGWVSRVCFEGDIPDFLEDFPEPITVTEPEQDSQLLKNQAVADETAVSACDTADAPHPLAVTLEPGARYFPRVIIPRAPTVKRDECVTGQRYHTGRLSGKIFCELNTLGPLFVPDTDYSAGVPVPISDEQLAECQLQAVFENTSKFNEFFATYPEETVTKLKDLLCAADDKWILAVKDITADLRQEIGEDTFQRIIRKAGHKTQRFHQINDEIGLPGASLRGMVLSNYQILTNSCYRNLKATEEITRRMPADEAKYRKAGRVTVSGDGAQKKYSIQEMEVLRLPIYDNMNTPDNMPDVAKQATTAKRCNNLMNEAAKTSRVELKARWREGQSKIKYQIIDALNKVDPIIQVISSSKQINPNNGKTGWGYVKYTGANVFAKSLVAPIDCLRKKDAGHVCCQVNLNPAWEASNFDILINEKCPVERQSGPRPTLRCKGQDSAWYTLTKRSERIFTDKKPVPDPINIPPREVKRYNELRDSYKKNTAHVPKPLQTFFNQESLANGDLVYFEVNQFGEASQLTPVSISRTTDLFPIGGRLPQGHKDLFPCTAMCLSECKNCVPASFCEFHSRSHEKLCPACSLAGTTGNRGRIKFSEAWLSGLPKWHSVSQDNVGRGLGVTMPRLERSRRTWHLPTKDAYLLGQSIYLNHPVPAILPSDQVPSENNQTVEPLGPKNIFSFQLAFDNLSIEELGLLLYSLELESGMAHRLGRGRALGMGSVQISVKDIQIRDNKSFLFSSNISKKSEWIQCGKDEFAQEAWFGESWDNIDHIQRLRQALTIPVKGDVGCIRYPKLEAEGGMPDYIKLRKRLTPLCDREEPVRYRINPVQLARMILPFVPWHGACPALLNEQVMIEAKRLTELXXXDRANWPC

SEQ ID NO: 9 - protein, 1367 aa, unidentified
  REGION 1..1367: note = source = /note="Description of Unknown: Deltaproteobacteria bacterium sequence"
ASEDDDTPTLRKVLKDEINGQEDMWRKFCEALGNSLYDLSKKAKERKRTEALPRLLGETEIYGLPMRENKEDEPLPSSLTYKFKWLIAGELRAETPFFFGTEVQEGQTSATILLNRDGYFRLPRSVIRGALRRDLRLVMGNDGCNMPIGGQMCECGVCRVMRHIVIEDGLSDCKIPPEVRHRIRLNCHTGTVEEGALFDMETGYQGMTFPFRLYCETENSDLDSYLWEVLNNWQNGQSLFGGDTGTGFGRFELTEPKVFLWNFSKKEKHEAYLLNRGFKGQMPVQDVKTKSFKTKTWFQIHRELDISPKKLPWYSTDYRFNVTSPLISRDPIGAMLDPRNTDAIMVRKTVFCPDPNAKNRPAPATVYMIKGESIRGILRSIVVRNEELYDTDHEDCDCILCRLFGSIHQQGSLRFEDAEVQNSVSDKKMDHVAIDRFTGGGVDQMKFDDYPLPGCPAQPLILEGKFWVKDDIDDESKSALEKAFADFRDGLVSLGGLGAIGYGQIGDFELIGGSADWLNLPKPEENRTDVPCGDRSAQGPEIKISLDADKIYHPHFFLKPSDKNVYRERELVSHAKKKGPDGKSLFTGKITCRLSTEGPVFIPDTDLGEDYFEMQASHKKHKNYGFFRINGNVAIPGSSIRGMISSVFEALTNSCFRVFDQERYLSRSEKPDPTELTKYYPGKVKRDGNKFFILKMKDFFRLPLYDFDFEGEAESLRPNYDEDRNEEENKGKNKNTQKVKNAVEFNIKMAGFAKHNRDFLKKYKEQEIKDIFMGKKKVYFTAGKHKPNEAHDNDKIALLTKGSNKKAEKGYFKFTGPGMVNVKAGVEGEECDFHIDESDPDVYWNMSSILPHNQIKWRPSQKKEYPRPVLKCVKDGTEYVMLKRSEHVFAEASSEDSYPVPGKVRKQFNSISRDNVQNTDHLSSMFQSRRLHDELSHGDLVYFRHDEKRKVTDIAYVRVSRTVDDRPMGKRFKNESLRPCNHVCVEGCDECPDRCKELEDYFSPHPEGLCPACHLFGTTDYKGRVSFGLGWHESNTPKWYMPEDNSQKGSHLTLPLLERPRPTWSMPNKKSEIPGRKFYVHHPWSVDKIRNRQFDPAKEKQPDDVIKPNENNRTVEPLGKGNEFTFEVRFNNLREWELGLLLYSLELEDNMAHKLGMGKALGMGSARIKAEAIELRCESAGQNAELKDKAAFVRKGFEFLEIDKPGENDPMNFDHIRQLRELLWFLPENVSANVRYPMLEKEDDGTPGYTDFIKQEEPSTGKRNPSYLSSEKRRNILQTPWKHWYLIPPFQASAQSETVFEGTVKWFDDKKGFGFIKINDGGKDVFVHHSSIVGTGFKSLNEGDSVAFKMGVGPKGPCAEKVKKIGN

SEQ ID NO: 10 - protein, 1322 aa, unidentified
  REGION 1..1322: note = source = /note="Description of Unknown: Syntrophorhabdaceae bacterium PtaU1.Bin034 sequence"
MRRQRLLGDAEYYGGTGREQPASIVISTDSDPDHKVYEWIITGQLKAETGFFFGTKAGAGGHTDLSILLGKDGHYRVPRSVFRGALRRDLRVAFGAGCRVEVGRERPCECPVCKVMRQITVMDTISSYREAPEIRQRIRLNPYTGTVDKGALFDMEVGPEGIEFPFVLRFRGSKSFPSELAAVIGSWTKGTAWLGGAAATGKGRFSLLGLSIHKWNLSTAEGRKSYLAAYGLRDAADKTVKRLSIDKGGKGDVGLPAGLERDALPSSVREPLWKKLVCTVDFSSPLLLADPIAALLGVEGDERIGFDNIAYEKRRYNGETNTTESIPAVKGETFRGIVRTALGKRHGNLTRDHEDCRCRLCAVFGKEQEAGKIRFEDLMPVGAWTRKHLDHVAIDRFHGGAEENMKFDTYALAASPTNPLRMKGLIWVRSDLFETGHDGPTPPYVKDIIDALADVKRGLYPVGGKTGSGYGWIKDVTIDGLPQGLSLPPAEERVDGVNEVPPYNYSAPPDLPSAAEGEYFFPHVFIKPYDKVDRVSRLTGHDRFRQGRITGRITCTLKTLTPLIIPDSEGIQTDATGHKMCKFFSVAGKPMIPGSEIRGMISSVYEALTNSCFRVFDEEKYLTRRVQPKKGAKSSELVPGIIVWGQNGGLAVQQVKNAYRVPLYDDPAVTSAIPTEAQKNKERWESVPSVNLQGALDWNLTTANIARDNRTFLNSRPEEKDAILSGTKPISFELEGTNPNDMLVRLVPDGVDGAHSGYLKFTGLNMVLKANKKTSRKLAPSEEDVRTLAILHNDFDSRRDWRRPPNSQRYFPRSVLRFSLERSTYTIPKRCERVFEGTCGEPYSVPSDVERQYNSIIDDISKNYGRISETYLTKTANRKLTVGDLVYFIADLDKNMATHILPVFISRISDEKPLGELLPFSGKLIPCEGEPPTILKKMAPSLLTEAWRTLISTHLEGFCPACRLFGTTSYKGRIRFGFAEHTGTPKWLREELDWARPFLTLPIQERPRPTWSVPDDKSEVPGRKFYLHHHGGNRIVESNLRNRPEVNQTKNNSSVEPISAGNTFTFDVCFENLEAWELGLLLYCLELSPKLAHKLGRAKAFGFGSVKIHVERIEERTTDGAYQDVTAVKKNGWITTGHDKLREWFHRDDWEDVDHIRNLRTVLRFPDADQEHDVRYPELKANNGVSGYVELRDKMTASERQESLRTPWYRWFPQNGTGGSGRHEQAATSQEQDTAKDESVLSATQRRQAVIDVSDPDERLSGTVESFDRQKGDGYIGCGVRQFYVRLEDIRSRTALCEGQVVTFRARKEWEGHEAYDVEIDQ

SEQ ID NO: 11 - protein, 476 aa, unidentified
  REGION 1..476: note = source = /note="Description of Unknown: Deltaproteobacteria bacterium sequence"
MLEKALADFRDGVVSLGGLGAIGYGRIGDFEVAEESGTWLKIPEKKLPEDSVQCGERYRFSSDPATRFEKEKIYYPHYFLKPSDEDVRRETRLVSHVYQEDTDGKTRLLTGTIRCRLTTEGPIFIPDTDDPKEDYFQMEIEGHKSYGFFRINEQVAIPGSSIRGMVSSVFEALTNSCFRVFDQKRYLSRSTKPDPRELEKYLPGKVKRIDNKWVLLELEDIFRLPLYDLKDVGPKSLDSAYGLEKFKNEKRFRLKKIENAVAFNKKMAGYAKHTREFLKNNYTETELGKILRGEMKVWFTIGHKPNSAHDNDKIALLTKKTNKRAKSGFIKFTGPSMVNIKADASNSDCEECRFDMKSEDKDGLIFHNAIECRPSQKKEYPRPVLKCVKEAVEYTMIKRCEQVFSEGKKPPRSYSIPDKSRRQYNGILKDNRDNTEHIPSFFRNRMKNKELSDEDLVYFRYKGKKVTNIAPVRVSR

SEQ ID NO: 12 - protein, 437 aa, unidentified
  REGION 1..437: note = source = /note="Description of Unknown: Deltaproteobacteria bacterium sequence"
QYNLPLNPDAFPKFRWIVTGHLRAETPFFFGRGEIKDRTIEEETEQTSKTILLNKDNFFRLPRSVIRGALRRDLRLVIGNGCNTPVGGKFCECDVCRIMRHVVVEDTISSCRKPPEIRYNIRLNGHTRTVEEGALFDTETGYQGMRFPFRLCFETRASEFDPDTSEPIPKFDPYLSEVMKHWKAGQAVFGGDTGAGFGRFRLEGDIRFFSMDVAKKEEYDPYLLARGFKGMSSQEILEKIGSGRTYDWNSVPKIALNIPPNKLPWKEICYTIEVISPLISRDPIRAMMDPRNTDTIMIRKRVFVPDGKGGTLPEPESRYFIKSETLRGILRSLVGGNKTADGEYLCDLDHEDCDCVQCRLFGSIHQQGCLRFEDAEVWNSVRDKKMDHVAIDRFTGGALDQMKFDDYPLLGCPEYPVILGGRFWIRDDISDKEKEIA

SEQ ID NO: 13 - protein, 350 aa, unidentified
  REGION 1..350: note = source = /note="Description of Unknown: Syntrophorhabdaceae bacterium PtaU1.Bin034 sequence"
MESIPVTLTFLEPYRVVEWYANEDRRSAERYLRGQSFARWHRKKNDKKGRPYITGTLLRSAAIRAAEELLSLSGGVWDGQHCCKGQFLSGGVKPEYMRKRPTYIWAEKEGACSAPDYCPFCIFLGDRDQAEKKAESQNGYPDKSYHIRFGNLSLPDPPPLLDLKEVAVERTLNRVDFQTAKAHDYFKVWEISHEDLGVYTGQIVIHYTGPWQEKVKSLLEGSLRFVDRLCGALCKIEMAPKPARPLPKSLSVDMTEHAKIIVTAFDDAKKAEKVRGLADAMRSMGSKGPTILDKLPAGHDDRDHHTWDVTIVDKTPLRTYLKGVLRADDAASWPALCKALGNALYDVSQG

SEQ ID NO: 14 - protein, 348 aa, unidentified
  REGION 1..348: note = source = /note="Description of Unknown: Nitrospirae bacterium sequence"
EKQGFRDKGFNIVGSLKDAIGKEIGLREISLRPAKEETMPRWQCVEYTIIVNSPLHTADPIEALLHSGNYDSVVYKKTVVRNGNIKQIPVFKGETIRGIVRTAFARILRTENVEFDEEHEDCTCPLCQVFGNEHRAGRVRFEDLVIEGYTSEKKFDHVSIDRFTGGAAEKRKFDDLSLKGSPRRPIVLRGKVWIRNDMDSKGIEKLKQAFMDIRDGLYPLGSRGGIGYGWVTDLKIENTEVEEFRLDKVSTTEGSGPATEEFNFPSLPEIQLNKDAVYHPHYFIRPHEKVNREIRPVGHERFHDDLLTGRIKCTLKTLTPLIIPDTEDPDAFGLQAEHKGHQNFRFFR

SEQ ID NO: 15 - protein, 1528 aa, unidentified
  REGION 1..1528: note = source = /note="Description of Unknown: Hydrothermal vent sediment bacterial communities from Southern Trench, Guaymas Basin, Mexico - 4870-07-3-4_MG sequence"
MKSIPITLTFLEPYRILPWAEKGKRDKKEYLRGANYVRLHKDKNGKFKPYITGTLIRSAVLSAIEMLLDITNGEWNGKECCLAKFHTEGEKPSFLRKKPIYIRAEKDEICTSRETACPLCLILGRFDKAEKKEKDKEKFDVHFSNLNLYSSKEFSTIEELAPKRALNRIEQYTGKAQDYFTVYEALNKEFWTFKGRIRIKEDIYDKVTDLLFSALRCVEKIAGALCRIEIDKEPSQQKGFVKRQLSKQAKEDIEKIFQVVKDAQKLRLLSDCFRELTRMANKDELALPLGPEDDGHYLWDKIKVEGKTLRIFLRNCFSQYKDNWLCFCDEASKKGYQKYREKRHKLTDRELPTATPKHFAEKKDPQISPIYIDKDDKVYEWIIVGRLIAQTPFHFGDEEKAEGAILLTPDNRFRLPRTALRGILRRDLKLAGASACEVEVGRSEPCPCDVCKIMRRVTLLDTVSEDLRDFLPELRKRIRINPQSGTVAEGALFDTEVGPEGLSFPFVLRYKCEKLPDSLTTVLCWWQEGLAFLSGESATGKGRFRLEINGAFVWDLQKGLFNYIKNHGFRGEERLFLEGNEAELEKMGIQINTELLQPEMIKKEKNFTDFPYDLIKYQLNISSPLLLNDPIRAIALYEGEGKAPDAVFFKKYVFENGKIEEKPCFKAESIRGIFRTAVGRIKNVLTKNHEDCICVLCHLFGNVHETGRLKFEDLKIVSGQEEKFFDHVAIDRFLGGAKEKYKFDDKPIIGAPDTPIVLEGKIWVKKDINDEAKETLSQAFSDINTGIYYLGANGSIGYGWIEEVKALKAPSWLKIKEKPNFEKDTSLNISAIMNEFKKDIQTLNLDKTYLPYGFLKLLEKVKRTSSPITHERFYENHLTGFIECSLKVLSPLIIPDTETPEKEENGHKYYHFLKIDNKPIIPGAEIRGAVSSIYEALTNSCFRVFGEKKVLSWRMEGKDAKEFMPGRVSKKKGKLYMVKMQALRLPVYDNPALANEIRSGSIYEKYKNSKVEIIFFQTVEGIRKFLRGNFNNVEWKKVLVTGIDPLAILPSQKIPGNDKWVKNLQSKISPVRGYFKFTGPNKIETKRREEEKDEKLRTKANKVSCLQKDKWYEAMHNHVEYKQDYTPPNSPKTEPLERPRNIPCFVCSDKEKIYRMTKRCERVFVSLGENAPKYEIPISAIKRYEVILSAYRENWERNKTPELFRTRLPGDGRTLNEDDLVYFRADENEKVKDIIPVCISRIVDEVPLIKRLSQELWPCVLAECPLLGFECKKCELEGLPEKIWFRINKDGLCPACRLFGTQIYKSRVRFSFAYAKNWKFYDGYITLPRLESPRATWLILKEKDKHYIKYKVCGRKFYLHNSTYEDIINNSKKEKEKKTENNASFEVLKEGEFTFKVYFENLENWELGLLLLSLTGLGEAIKIGHAKPLGFGSVKIEAKKIYFREEAGKFHPCEKADEYLKKGLNKLTSWFGKNEINEHMRNLLLFMTYYQNLPKVKYPDFDGYAKWRCSYVEQDKVEYFQNRWIVAS

SEQ ID NO: 16 - protein, 1652 aa, unidentified
  REGION 1..1652: note = source = /note="Description of Unknown: Sorted cell/s from Southern Trench hydrothermal vent microbial mat, Guaymas Basin, Mexico - 6X_4868_18_01 sequence"
MIINITVKFLGPFRMLEWTDPDNRNRKNREFMRGQAFARWHNSNPQKGSQPYITGTLVRSAVIRSAENLLMLSEGKVGKEKCCPGEFRTENRKKRDAMLHLRQRSTLQWKTDKPLCNGKSLCPICELLGRRIGKTDEVKKKGDFRIHFGNLTPLNRYDDPSDIGTQRTLNRVDYATGKAHDFFKVWEIDHSLLSVFQGKISIADNIGDGATKLLEDSLRFTDRLCGAICVISYDCIENSDGKENGKTGEAAHIMGESDAGKTDAENIANAIADMMGTAGEPEKLRILADAVRALRIGKNTVSQLPLDHEGKENHHLWDIGEGKSIRELLLEKAESLPSDQWRKFCEDVGEILYLKSKDPTGGLTVSQRILGDEAFWSKADRQLNPSAVSIPVTTETLICGKLISETPFFFGTEIEDAKHTNLKVLLDRQNRYRLPRSAIRGVLRRDLRTAFGGKGCNVELGGRPCLCDVCRIMRGITIMDARSEYAEPPEIRHRIRLNPYTGTVAEGALFDMELGPQGLSFDFILRYRGKGKSIPKALRNVLKWWTKGQAFLSGAASTGKGIFRLDDLKYISFDLSDKDKRKDYLDNYGWRNRIEALSLEKMPLDRMNDYAEPLWQKVSVEIEIGSPFLNGDPIRALIEKDGSDIVSFRKYADDSGKEVYAYKAESFRGVVRAALARQHFDKEGKPLDKEGKPLLTLIHQDCECLICRLFGSEHETGRLRFEDLLFDPQPEPMIFDHVAIDRFTGGAVDKKKFDDCSLPGTPGHPLTLKGCFWIRKELEKPDEDKSEREALSKALADIHNGLYPLGGKGAIGYGQVMNLKIKGAGDVIKAALQSESSRMSASEPEHKKPDSGLKLSFDDKKAVYYPHYFLKPAAEEVNRKPIPTGHETLNSGLLTGKIRCRLTTRTPLIVPDTSNDDFFQTGVEGHESYAFFSVNGDIMLPGSEIRGMLSSVYEALTNSCFRVFDEGYRLSWRMEADRNVLMQFKPGRVTDNGLRIEEMKEYRYPFYDRDCSDKKSQEAYFDEWERSITLTDDSLEKMAERKGDISPKDLKVLKSLKGKNYKSTEGLLAAFKDKGGDTGGNILGLIFKYAERIGDVPRYEHPTDTDRMMLSLSEYNRNQKSDGKRAYKIIKPASKLGKGAYFMFAGTSVENKRICNPACTDKANKSVKGYLKISGPNKLEKYNISEPELDGVPEDRNCQIIHNRIYLRKIFVANAKKRKERDRLVGEFACYDPEKKVTYSMTKRCERIFIKDRGRTLPITHEASELFEILVQEYRENAKRQDTPEVFQTLLPDNGRLNPGDLVYFREEKGKTVEIIPVRISRKIDDSPIGKRLREDLRPCHGEWIEGDDLSQLSEYPEKKLFTRNTEGLCPACRLFGTGAYKGRLRFGFAKLENDPKWLMKNSDGPSHGGPLTLPLLERPRPTWSMPDDTLNRLKKDGKQEPKKQKGKKGPQVPGRKFYVHHDGWKEINCGCHPTTKENIVQNQNNRTVEPLDKGNTFSFEICFENLEPYELGLLLYTLELEKGLAHKLGMAKPMGFGSIDIEVENVSLRTDSGQWKDANEQISEWTDKGKKDAGKWFKTDWEAAEHIKNLKKLLFLPGEEQNPRVIYPALKQKDIPNSRLPGYEELKKNLNMEKRKEMLTTPWAPWHPIKK

SEQ ID NO: 17 - protein, 1723 aa, unidentified
  REGION 1..1723: note = source = /note="Description of Unknown: Hydrothermal vent microbial mat bacterial communities from Southern Trench, Guaymas Basin, Mexico - 4869-18-0-1_MG sequence"
MTQITIQVTFFHPFRVVPWNHRDHRKTDRKYLRGGTFAKWHCTASEGKSGRPYITGTLLRSALFAEIEKLIAFHDPFKCCRGKDKTENGNAKPLFLRRRPRADCDPCGTCPLCLLMGRSDTVRRDAKKQKKDWSVHFCNLREATERSFNWKETAIERIVNRVDPSSGKAKDYMRIWEIDPLVCSQFNGIITINLDTDNAGKVKLLMAAGLAQINILAGSICRADIISEDHDALIKQFMAIDVREPEVSTSFPLQDDELNNAPAGCGDDEISTDQPVGHNLVDRVRISKIAESIEDVFSQEQKAQQLRRMADAIRDLRRSKPDETTLDALPKGKTDKDNSVWDKPLKKDILPSPRMPASEDDDTPTLRKVLKDEINGQEDMWRKFCEALGNSLYDLSKKAKERKRTEALPRLLGETEIYGLPMRENKEDEPLPSSLTYKFKWLIAGELRAETPFFFGTEVQEGQTSATILLNRDGYFRLPRSVIRGALRRDLRLVMGNDGCNMPIGGQMCECGVCRVMRHIVIEDGLSDCKIPPEVRHRIRLNCHTGTVEEGALFDMETGYQGMTFPFRLYCETENSDLDSYLWEVLNNWQNGQSLFGGDTGTGFGRFELTEPKVFLWNFSKKEKHEAYLLNRGFKGQMPVQDVKTKSFKTKTWFQIHRELDISPKKLPWYSTDYRFNVTSPLISRDPIGAMLDPRNTDAIMVRKTVFCPDPNAKNRPAPATVYMIKGESIRGILRSIVVRNEELYDTDHEDCDCILCRLFGSIHQQGSLRFEDAEVQNSVSDKKMDHVAIDRFTGGGVDQMKFDDYPLPGCPAQPLILEGKFWVKDDIDDESKSALEKAFADFRDGLVSLGGLGAIGYGQIGDFELIGGSADWLNLPKPEENRTDVPCGDRSAQGPEIKISLDADKIYHPHFFLKPSDKNVYRERELVSHAKKKGPDGKSLFTGKITCRLSTEGPVFIPDTDLGEDYFEMQASHKKHKNYGFFRINGNVAIPGSSIRGMISSVFEALTNSCFRVFDQERYLSRSEKPDPTELTKYYPGKVKRDGNKFFILKMKDFFRLPLYDFDFEGEAESLRPNYDEDRNEEENKGKNKNTQKVKNAVEFNIKMAGFAKHNRDFLKKYKEQEIKDIFMGKKKVYFTAGKHKPNEAHDNDKIALLTKGSNKKAEKGYFKFTGPGMVNVKAGVEGEECDFHIDESDPDVYWNMSSILPHNQIKWRPSQKKEYPRPVLKCVKDGTEYVMLKRSEHVFAEASSEDSYPVPGKVRKQFNSISRDNVQNTDHLSSMFQSRRLHDELSHGDLVYFRHDEKRKVTDIAYVRVSRTVDDRPMGKRFKNESLRPCNHVCVEGCDECPDRCKELEDYFSPHPEGLCPACHLFGTTDYKGRVSFGLGWHESNTPKWYMPEDNSQKGSHLTLPLLERPRPTWSMPNKKSEIPGRKFYVHHPWSVDKIRNRQFDPAKEKQPDDVIKPNENNRTVEPLGKGNEFTFEVRFNNLREWELGLLLYSLELEDNMAHKLGMGKALGMGSARIKAEAIELRCESAGQNAELKDKAAFVRKGFEFLEIDKPGENDPMNFDHIRQLRELLWFLPENVSANVRYPMLEKEDDGTPGYTDFIKQEEPSTGKRNPSYLSSEKRRNILQTPWKHWYLIPPFQASAQSETVFEGTVKWFDDKKGFGFIKINDGGKDVFVHHSSIVGTGFKSLNEGDSVAFKMGVGPKGPCAEKVKKIGN

SEQ ID NO: 18 - protein, 1548 aa, unidentified
  REGION 1..1548: note = source = /note="Description of Unknown: Hydrothermal sediment microbial communities from Guaymas Basin, California, USA 4484. Combined assembly of Gp0115313 and Gp0146561 sequence"
MTKIPISLTFLEPFRLVDWVSESERDKSEFLRGLSFARWHRIKNQREDENQGRPYITGTLLRSAVIKAAEELIFLNGGKWQSEECCNGQFKGSKAKYRKVECPRRRHRATLKWTDNTCSDYHNACPFCLLLGCLKPNSKENSDIHFSNLSLPNKQIFKNPPEIGIRRILNRVDFTTGKAQDYFYVWEVEHSMCPKFQGTVKINEDMPKYNVVKDLLISSIQFVDKLCGALCVIEIGKTKNYICQSFSSNIPEEEIKKLAQEIRDILKGEDALDKMRVLADTVLQMRTKGPEIVNELPRGIEKKGGHWLWDKLRLRKKFKEIANNYKDSWQELCEKLGNELYISYKELTGGIAVKKRIIGETEYRKIPEQEISFLPSKAGYSYEWIILGKLISENPFFFGKETKTEEQIDMQILLTKDGRYRLPRSVLRGALRRDLRLVIGSGCDVELGSKRPCPCPVCRIMRRVTLKDARSDYCKPPEVRKRIRINPLTGTVQKGALFTMEVAPEGISFPFQLRFRGEDKFHDALQNVLVWWKEGKLFLGGGASTGKGRFKLEIEHVLKWDLKNNFHSYLQYKGLRDKGDFNSIKEIEGLKVETEEFKVKKPFPWSCVEYTIFIESPFVSGDPVEAVLDSSNTDLVTFKKYKLEESKEVFAIKGESIRGVFRTAVGKNEGKLTTENEHEDCTCILCRLFGNEHETGKVRFEDLELINDSAPKRLDHVAIDRFTGGAKEQAKFDDSPLIGSPDSPLEFTGIVWVRDDIDEEEKKALKSAFLDIKSGYYPLGGKKGVGYGWVSNLKIESGPEWLRLEVQEKSSQENVLSPVILSEVMDIEFNPPKIDENGVYFPYAFLRPLNEVKRTREPIGHNEWKKSLISGYLTCRLELLTPLIIPDTSEEVIKEKVNNGEHPVYKFFRLGGHLCIPAAEIRGMISSVYEALTNSCFRVFDEKRLISWRMTAEEAKRPDPKKSEEQNRMRFRPGRIIKKDKKFYAQEMLELRIPVYDNKDKRNEISQNDPTRPSEYNHPTEPERIFFSNAEKIRNFLKRNSNYLHGSTPLLFRQWSISNRYDKIALIGNKSQGHLKFTGPNKIEVSEGTKCPKYETIPGRDEWDKAVHNYVEPGKFVTVISRKKGQKPKAVQRRRNVPAFCCYDYNTNRCFVMNKRCERVFKVSRDKPKYEIPPDAIRRYEHVLRKYRENWERYDIPEVFRTRLPGDGETLNEGDLVYFRLDENNRVLDIIPVSISRISDTQYLGRRLPDHLRSCVRECLYEGWGDCKPCKLSLFPEKMWIRINPEGLCPACHLFGTQVYKGRVRFGFARAGSNWKFREEQLTLPRFETPRPTWVIPKRKDEYQIPGRKFYLHHNGWEEIYKKNKKNEIKKEKNNATFEVLKQGTFYFKVFFENLELWELGLLIFSAELGGEEFAHKLGHGKALGFGSVKISVDKIILRRDPGQFEQRGQKFKRDAVDKGFCVLENRFGKTNFKIYLNNFLQLLYWPNNKKVKVRYPYLRQEDDPEKLPGYVELKKHQMLKDDNRYSLFARPRAVWLKWTEMVQRDKS

SEQ ID NO: 19 - protein, 1720 aa, unidentified
  REGION 1..1720: note = source = /note="Description of Unknown: Hydrocarbon resource environments microbial communities from Canada and USA sequence"
MSVEEFYVRLTFLEPFRVVPWVRNGDERKGDRIYQRGGTYARWHKINDSHGQPYITGTMLRSAVLREIENTLTLHNTYGCCPGGTRTTEGKLEKPLYLRRRDGFEFENHAEKPCSEEDPCPLCLIQGRFDKLRRDEKKQFVRQGNISFCSVNFSNLNISSGIKSFSWEEIAVSRVVNRVDPNSGKAKDFFRVWEIDHKLCPNFLGKMSISLSEKLEDVKALLAVGLAQVNVLSGALCRVDIIDPETQKDTVHQHLIQQFVTRIQDKEKGDAADIPAFTLPPAGLSPSSNEWNDTIKSLAEKIRKIKELEQGQKLRQMADVIRELRRKTPAYLDQLPAGKPEGRESIWEKTPTGETLTLRQLLKSANVPGESWRAFCEELGEQLYRLEKNLYSHARPLPRLLGETEFYGQPARKSDDPPMIRASYRAFPSYVWVLDGILRAETPFYFGTETSEGQTSQAIILCPDGSYRLPRSLLRGVIRRDLRAILGTGCNVSLGKVRPCSCPVCEIMRRITVQQGVSSYREPAEVRQRIRSNPHTGTVEEGALFDLETGPQGMTFPFRLYFRTRSPYIDRALWLTINHWQEGKAIFGGDIGVGMGRFRLENLQIRSADLVSRRDFSLYLRARGLKGLSREEVTRIGLNEEQWEAVMADDPGTHYNPFPWEKISYTLLIHSPLISNDPIAAMLDHDNKDAVMVQKTVLFVDESGNYSQMPHHFLKGSGIRGACRFLLGRKDAPNENGLTYFEADHEECDCLLCSLFGSKHYQGKLRFEDAELQDEVEAIKCDHVAIDRFHGGTVHRMKYDDYPLPGSPNRPLRIKGNIWVKRDLSDTEKEAVKDVLTELRDGLIPLGANGGAGYGRIQRLMIDDGPGWLALPERKEDERPQPSFSPVSLGPVHVNLKSGSDTADVYYYHPHYFLEPPSQTVSRELDIISHARTRDSGGEALLTGRILCRLITRGPIFIPDTNNDNAFGLEGGIGHKNYRFFRINDELAIPGSELRGMVSSVYEALTNSCFRIMEEGRYLSRRMGADEFKDFHPGIVVDGAKIREMKRYRLPLYDTPDKTSRTKEMTCPELFTRKDGRPERAKKFNEEIAKVAVQNRAYLLSLDEKERREVLLGNREVTFDECPDDEYSDDEYSELKYAQKYKDFIAVLKKNGQKRGYIKFTGPNTANKKNEDAPDKNYRSDWDPFKLNILLESDPECRVSNIHCYPRPLLVCIKDKAEYRIHKRCEAIFCSIGSPSDLYDIPQKVSNQYRTILQDYNDNTGKIVEIFRTQIKHDQLTTGDLVYFKPAANGQVNAVIPVSISRKTDENPLAKRFKNDSLRPCAGLCVEDCNECPARCKKVADYFNPHPRGLCPACHLFGTTFYKGRVRFGFAWLTGEDGAPRWYKGPDPCDSGKGRPMTIPLLERPRPTWSIPDNSFDIPGRKFYVHHPYSVDGIDGETRTPNNRTIEPLAEGNEFVFDIDFENLRDWELGLLLYSLELEDSLAHKLGLGKPLGFGTVQINIRGISLKNGSKGWDTKTGDDKNQWIKKGFAHLGIDIKEANERPYIKQLRELLWVPTGDNLPHVRYPELESKTKDVPGYTSLLKEKDLADRVSLLKAPWKPWKPWSGTAPHPDKGTNRLRASIVERDRIQRKTDTAKPEKKEETKVGKSSSSDIEKRYVGTVKWFNDKKGYGFILYGTDEEIFVHRSGVADNSIPKEGQKVGFRIERGARGSHAVEVKAIE

SEQ ID NO: 20 - protein, 1610 aa, unidentified
  REGION 1..1610: note = source = /note="Description of Unknown: Freshwater metagenome sequence"
MPRFQLSLTFFDEPFRLIEWTDKSNRNSANTQWMRGQGFARWHKITLEKGFPFVTGTAVRSKIIREVEALLSRNKGTWNGIPCCSGFFDTKGPSPTHLRYRPTLEWEYGKTVCTSEADVCPLCLLLGRFDQAGKKSDTPCQSTDYHVHWENLSAGVAQYRLEDIAQKRTSNRVDFFSKKAHDHYGVWEVTAVKNLLGYIYISDAITESHQKTVISLLKAALSFTDTLCGANCKLELSDEPVDSIHSNQSASNFNPHSGAAPSQCSQSMPPFNMDQETKELANTLCKAFTGNMRHLRTLADAVREMRRMSPGISSLPRGRLNKEGEITAHYLWDERIDEKTIRQVLEDTIELSPARSIIYKNWISFCNQLGQKLYERAKDNDPILERKRPLGEAAFSKVPTSSHAPRHDMNSRVKGGFTREWIIVGTLRALTPFYMGTGSQAGKQTSMPTLQDSNDHFRLPRTALRGALRRDINQASDGMGCVVELGPHNLCSCPVCQVLRQIRLLDTKSKFSMPPAIRQKICKNPVLSIVNEGSLFDVELGIEGETFPFVMRYRGGAKIPDTIITVLSWWKNERLFIGGESGTGRGRFVLECPRIFCWDVEKGQNDYIQYHGFRNKEDELLSVYSTVSGLAEKNDVNLNNARDFSFDKICWEVQFDGPVLTGDPLAALFHGNTDSVFYKKPILKSGEKEPSYQWAIKSDTVRGLIRSAFGKRDALLIKSHEDCDCLLCEAFGSKHHEGKLRFEDLTPKSDEIKTYRMDHVAIDRISGGAVDQCKYDDEPLVGTSKHPLVFKGMFWINRDSSVEMQRALIAAFKEIRDGLYPLGSNGGTGYGWISHLAITNGPDWLNLEEVPLPQPTADIPVEECTAEPYPKFQKPDLDQNAVYYPHYFLQPGKPAERERHPVSHDHIDDKLLTGRLVCTLTTKTPLIIPDTQTNTMLPPNDAPEGHKSFRFFRIDDEVLIPGSEIRGMVSTVFEALTGSCFRVINQKAHLSWRINADMAKHYRPGRIIQNNEKMFIQPYKMFRLPFYAGFDPRNCLSEKQLLGIEPVKLWVKDFVASLVKPQTDIDIEWKEKIGFVRVTGPNKVEVDSSNTPDPSLPECESDWKDIHITEDGSTPSKNDRVYRCQLKGVTYTVAKWCEAFWVKDEGKKPITVNAEAINRYHLIMKSYQDNPQSPPIIFRSLPVLNYKQDQKIIGSMIFYRESAKSDKIVNEIIPVKISRTADTELLAKHLPNNDFLPCAATCLNECDTCNAKTCKFLPLYREGYPVNGLCPSCHLFGTTGYQGRVRFGFAKMNGNAKFCQGGERPEDRAVTLPLQERPKLTWVMPNENSTIPGRKFFLHHQGWKKIVDEGKNPINGDVIEPDANNRTVEPLAAGNDFSFEVFFENLREWELGLLRYTLELESELAHKLGMGKAFGFGSVKIKIKSVDLRKQGEWEKATNTLVSEDKKSSWYNIHTVNNLRTALYYVEDDKIQVNYPKLKKDNESDNRPGYVEMKKTAFPVRDILTTPWWPWWPPTPPPMNQSGNQSYARSEEPARITESQPEVYKTGTVKFYKHDKKFGFITMDGRENIHFAGNQICRPETSLQSGDKVKFIEGENYKGPTALKVERLKG

SEQ ID NO: 21 - protein, 1575 aa, unidentified
  REGION 1..1575: note = source = /note="Description of Unknown: soil metagenome sequence"
MRLKINIHFLEPFRLIEWHEQDRRNKGNSRWQRGQSFARWHRRKDNDQGRPYITGTLLRSVVIRAVEEELARPDTAWQSCGGLFITPDGQTKPQHLRHRATVRARQTAKDKCADRQSACPFCLLLGRFDQVGKDGDKKGEGLRFDVRFSNLDLPKDFSPRDFDGPQEIGSRRTINRVDDETGKAHDFFSIWEVDAVREFQGEIVLAADLPSRDQVESLLHHALGFVDRLCGARCVISIADQKPAEREERTVAAGDEKATIADYDQVKGLPYTRLRPLADAVRNLRQLDLAELNKPDGKFLPPGRVNKDGRRVPHYVWDIPLGKGDTLRKRLEFLAASCEGDQAKWRNICESEGQALYEKSKKLKDSPAAPGRHLGAAEQVRPPQPPVSYSEESINSDLPLAEWIITGTLRAETPFAIGMDAPIDDDQTSSRTLVDRDGRYRLPRSTLRGILRRDLSLASGDQGCQVRLGPERPCTCPVCLILRQVVIADTVSETTVPADIRQRIRRNPITGTAADGGLFDTERGPKGAGFPFSLRYRGHAPMPKALRTVLQWWSAGKCFAGSDGGVGCGRFALDNLEVYRWDLGTFAFRQAYSENNGLRSPEEEFDLAVIHELAEGLAKEDGQKILKGTEPFTCWQERSWQFSFTGPLLQGDPLAALNSDTADIISFRRTVVDNGEVLREPVLRGEGLRGLLRTAVGRVAGDDLLTRSHQDCKCEICQLFGSEHRAGILRFEDLPPVSPTTVADKRLDHVAIDRFDQSVVEKYDDRPLVGSPKQPLVFKGCFWVQTSGMTHQLTELLAQAWRDIAAGHYPVGGKGGIGYGWINSLVVDGEKITCRPDGDSISLTTVTGDIPPRPALTPPAGAIYYPHYFLPPNPEHKPKRSDKIIGHHTFATDPDSFTGRITCKLEVVTPLIVPDTEGEQPKDQHKNFPFFKINDEIMLPGAPLWAAVSQVYEALTNSCFRVMKQKRFLSWRMEAEDYKDFYPGRVLDGGKQIKKMGDKAIRMPLYDDSTATGSIKDDQLISDCCPKSDEKLQKALATNQKIALAAKHNQEYLAQLSPDEREEALQGLKKVSFWTESLANNEAPPFLIAKLGEERGKPKRAGYLKITGPNNANIANTNNPDDGGYIPSWKDQFDYSFRLLGPPRCLPNTKGNREYPRPGFTCVIDGKEYSLTKRCERIFEDISGGENQVVRAVTERVREQYREILASYRANAAGIAEGFRTRMYDTEELRENDLVYFKTAKQADGKERVVAISPVCISREADDRPLGKRLPAGFQPCSHVCLEDCNTCSAKNCPVPLYREGWPVNGLCPACRLFGAQMYKGRVNFGFARLPDDKQPETKTLTLPLLERPRPTWVLPKSVKGSNTEDATIPGRKFYLRHDGWRIVMAGTNPITGESIEKTANNATVEAIMPGATFTFDIVCENLDQQELGLLLYSLELEEGMSHTLGRGKPLGFGNVRIKVEKIEKRLSDGSRREMIPPKGAGLFMTDKVQDALRGLTEGGDWHQRPHISGLRRLLTRYPEIKARYPKLSQGEDKEPGYIELKSQKDENGVPIYNPNRELRVSENGPLPWFLLAKK

SEQ ID NO: 22 - protein, 1237 aa, unidentified
  REGION 1..1237: note = source = /note="Description of Unknown: groundwater metagenome sequence"
MSNQTRWIIEGTLELITPLHIGTGLDEKERDENKETRWLEAVALDHKGQPYIPGASLKGALRALAKRHDFRNLFDNKEVDGDFVRQAEFLSAWCVPDTDKGRLIQPRVAIDRVTGTAQDKKLFQTRLITPGTRFAMKIVVQNAVENEIADLLGLLNLLPDDPQFSLGAYANQGQGRVQWFGKIQTRCFGINEAKAWYEEIRKDESKCWTAFAKPKNVSTPPTPAKEAQLTLPLNLAFHTPFLVKQAGIKADDADAVPRRTHDDKIVLPASSLRGRLRTQSERILRTLGCETPQGHTAPAYRKGQPHDDLAVLLFGAAGWRGIVQTSDCIVEDKSIKTRRHEMLAIDRFTGGGKDGAKFNVDYVECPTLAGKLSLDLARLKNAKLKGGKDALLPALGLMTLMLRDLAEGDIPFGYGISKGYGQCRASSALGDWAELLKQHLGADSADTTVQALREYLGNPKGQELKLDPPSADATQAGVPAQQNAAKTQAQGAQEKFHNPYHFIPLSKPDISQWPEPQKLTEKGHSHDRYASLSGRIVCRLTTQTPLFIGSEQTTPTNPQAPKSLHPFKLNNGLAIPATSLRGMISSLFESVSNSNFRVLDEKTYSMRKTMQQSLSAMGRIVRHDQKLYLLPLTLPTLPQGPHGVYDLGEKWSAVFDWQPPPLRIYFDPPPRRTYQSQQPCYMKLSTVKYSESNPNQIIAGENLGALRFPRGNQNTQFLIGQSNQDECPITQAEYAQKSEDERNEYTSGWVRTLVKPGRDLPRSVKHHVFLPDVFIDAPPPVNDLYPIPDSVIQRFHDLADQVLASMNLKPEEIVDSTNLLPYTPVGRRSDSDCRDTRLQAGDLIFFDIDPPLHPGEKSQITEISFSSIWRSGIGKDHLLTTPDLLTNFDVNLQPHGMPGRTQSLSPAELLFGLVGTQNDQATTAYAGKVRIGFGLPEEGHNPRLDARITLKELSSPKPPSPALYFRKKSGKDEYVSKANLADKPEDYILRGRKMYLHAWRKNEQVVELSDTGHDGGVRPPWVSKFDESADEGNKRRVSIEPIAKDESFYFEVDFHNLSRTELAQLCATLYPNEKFEHRLGMGKPLGLGSIKITPLSLFLVNRSQRYATDGLDKPRYHAVWHTGTASEPRWPDHLQREQQGIAFEGVSTAPTVMSLAAEAKVSDDVKRALELLGNPDEITVPVHYPQLHNGLMESKQFDWFVQNDKSGRDQPANNRQHLSSFTKDTEKLEPLIRIMRR

SEQ ID NO: 23 - protein, 1291 aa, unidentified
  REGION 1..1291: note = source = /note="Description of Unknown: wastewater metagenome sequence"
MTTPSAPKSSLPALHWLIRAELEVLTPLHLGTGTDQRITPDAPDADPYWQADIALDADGRPYLPGASLKGALKALARRRQVDAPCLPLFGDLNRGGAPHPDCIPPRRTRAGLAEFRDALQSHATQADGPDTAQPRIAIDRITGSVVDKKLFHTQTVPVGTRFSVEIILRRADQNLAAQLVALLQHGPTDPDFRLGAHANLGFGRVGLYGNIDTRRFGPQQAQHWFAAAQTQADARWTDFAEAVTLTAPAPAPAQPAPHRLALPLSLTFHTPFLVKQPEHKHRKPQDNAPDGTPRQRGDRALLPGASLRGRLRSQAERILRTLGCKVAQGHAVPPVKNNTCPDPATLLFGTAGWRGLLRTDDCTGTAPATLVDHDMLAIDRFTGGGKDGAKFKLRYAECPTLEGQLSLDLSRLRSARLDGANAADTPWIALGLLTLVLRDLAEGDIPFGHGSAKGYGRCRAQGLPDRWRQALEAHFGPNADARALAALRAWCRTHATAALDAPCSLAGSAPTPAAAAPSGQAAPADAFHNPYHFIPFSQPDIDRWLSPDAHRKTGGHSRYRGLSGRLVCALTTVTPLFVGAAARTPASDQHPKPVAGFALQNQPAIPATSLRGLLSSLFESISGSNLRVLHPTPYSIRKTTKEALSAIGRIVERNGELKLYPLTLPTIHQNADNAYPVPARWRKVFYWESPVPLRVYFGSRKQTYDSRQPHYLPIQELSYLPNDSDCIAPDQGDLRFPSRDRDRKFLIGQCPISRYDCPIPETDLPKLSPQERPRYTRGWVRSLWTSNREKELPHTVKHQLFIPDPVETPAADDLLPIPQGVLDTFHALADLALAGQHWGKDETPADDQLLPFTPAGRQRHDADRPPRDADRQTRLQPGDLVCFDLGDDGAVSEISFSSIWREGLRLAGKPNLATTADLLAQVSPHLLPLGMPGRSARLSPVEQLFGVVEYRPPQTAKGTRKPTDAPAAYALAGKLQVGFGRPARPFEREPAVTLKELSTPKPPSPALYFRPKAGDGYVSKAKLASQPQDYAPAGRKHYLHALRRQGQVARLDNSGHVPSDGSGRPPWQSRFDGQEDSGNKRRVRVEPIPAGETFHFEIDFDNLSPTELEQLCATLLPHPAFEHRLGMGKPIGLGSVKLAVEGLLLVDRPRRYAEDEPNAPRHHRGWRANADAGWPDHLQGDSPAAPLEATEQPAALAERAMARVPADVRRALQLLGNPGAVAAPVHYPQVKDAQIEEKHYLWFVANDDEKTAGGNRHLPRLHANSPGLPTLPRLVKREKDHSSNTGKPRRK

SEQ ID NO: 24 - protein, 1801 aa, unidentified
  REGION 1..1801: note = source = /note="Description of Unknown: oral metagenome sequence"
MIPDLRSLVVHISFLTPYRQAPWFPPEKRRNNNRDWLRMQSYARWHKVAPEEGHPFITGTLLRSRVIRAVEEELCLANGIWRGVACCPGEFNSQAKKKPKHLRRRTTLQWYPEGAKSCSKQDGRENACPFCLLLDRFGGEKSEEGRKKNNDYDVHFSNLNPFYPGSSPKVWSGPEEIGRLRTLNRIDRLTTKAQDFFRIYEVDQVRDFFGTITLAGDLPRKVDVEFLLRRGLGFVSTLCGAQCEIKVVDLKKKQNNKEDSILPVSEVPFFLEPEVLAKMCQDVFPSGKLRMLADVILRLREEGPDNLTLPMGSQGLGGRLPHHLWDVPLVSKDRETQTLRSCLEKIAAQCKSEQTQFRLFCQKLGSSLFRINKGVYLAPNSKISPEPCLDPSKTIRTKGPVPGKQKHRFSLLPPFEWIITGTLKAQTPFFIPDEQGSHDHTSRKILLTRDFYYRLPRSLLRGIIRRDLHEATDKGGCRVELAPDVPCTCQVCRLLGRMLLADTTSTTKVAPDMRHRVGVDRSCGIVRDGALFDTEYGIEGVCFPLEIRYRGNKDLEGPIRQLLSWWQQGLLFLGGDFGIGKGRFRLENMKIHRWDLRDESARADYVQKCGLRRGVGDDTAINLEKDLSLNLPESGYPWKKHAWKLSFQVPLLTADPIMAQTRHEEDSVYFQKRIFTSDGRVVLVPALRGEGLRGLLRTAVSRAYGISLINDEHEDCDCPLCKIFGNEHHAGMLRFDDMVPVGTWNDKKIDHVSCSRFDASVVNKFDDRSLVGSPDSPLHFEGTFWLHRDFQNDVEIKTALQDFADGLYSIGGKGGIGYGWLFDMEIPRSLRKLNSGFREASSIQDALLDSAKEIPLSAPLTFTPVKGAVYNPYYYLPFPAEKPERCLVPPSHARLQSDRYTGCLTCELETVSPLLLPDTCREKDGNYKEYPSFRLNNTPMIPGAGLRAAVSQVYEVLTNSCIRIMDQGQTLSWRMSTSEHKDYQPGKITDNGRKIQPMGKQAIRLPLYDEVIHHVSTPGDTDDLEKLKAIVLELTRPWKELPEEQKKKRFEKCKNILDGRMLQQKELRALENSGFAYWRDKTSLTFDSFLKDAIEQEYPRYSGDYQRIKALVVNITLPWKLLKKEERHKRFDKCRRILKGQQPLTKDERKALEESGFANWHGRELLFDRFLKDENSCLIKAETTDRVIASVAKNNRDYLFEIKQQDFARYKRIIQGLERVPFSLRSLAKSKETSFQIACLGLRRGRFLRKGYLKISGPNNANVEISGGSHSNSGYSDIWDDPLDFSFRLSGKSELRPNTQKTREYPRPSFTCTVDGKQYTVNKRCERVFEDSAAPAIELPRMVREGYKGILTDYEQNAKHIPQGFQTRFSSYRELNDGDLVYYKTDSQGRVTDLAPVCLSRLADDRPLGKRLPEEYRPCAHVCLEECDPCTGKDCPVPIYREGYPARGFCPACQLFGTQMYKGRVRFSFGVPVNSTRSPQLKYVTLPSQERPRPTWVLPESCKGKEKDVPGRKFYLRHDGWREMWGDDDKPDSRPSSEECQDIIEGIGPGEKFHFRVAFENLDKNELGRLLYSLELDAGMNHHLGRGKAFGFGQVKIRVTKLERRLEPGQWRSEKICTDLPVTSSELVISSLKKVEERRKLLRLVMTPYKGLTACYPGLERENGRPGYTDLKMLATYDPYRELVVQIGSNQPLRPWYEPGKSFKPSPGNDCTGRGGSVSKSLISEPKVVPAIAPFCEGVVKWFNSVKGFGFIETKEQRDIFVHFSAIRGEGYKILEPGEKVRFEIGEGRKGPQAINVIRIR

SEQ ID NO: 25 - protein, 1322 aa, unidentified
  REGION 1..1322: note = source = /note="Description of Unknown: hydrothermal vent metagenome sequence"
MKMNKTWPFREHWEISGYLRTVTPLHIGSGRTVTRPELTVADRDELVDINAVVTDYTGKPYLPGSTIKGALHAWLQKRLKEESRTCLIQLFGQEEEAEEKKKNNHGGKAEFFDARVIFPHTGPGSLPYWDDCRQTYVAATVAIDNITRTARHRHLIHAEMVPPGVTFALTLAGPLDEEDIGLLLAALQGFNESPPALVIGAHTANGLGRFSWELSTVRRFGKEHLQGWLEAETRAMRTEAMQPLSREGVEDLLQDGIAQIDHNEDQVRLGLELCFDGPFLVNDPPTKKEQDDKKKRRSNTPNLRPLRDAIGRPCLPESSVRGALRAQAERIIRTMEGTCSEDNPAYKKEIHTDAEIEELSAVCRVFGAPGWKSLLEISDFEFVDGEDCDNIQEFVAIDRFTGGAKDKAKFNAEYIGSPRFTGTIALDKRRDLPDWGKGLLYLVLRDLAEGDITLGFGRSKGYGVCRAKIKNLDLLLPEKSVAALHKKFAISPADDKPATDQIAEDTTSGNLGISAGGPAQKTTESYEPPNPSGPGTFHNPYHFVPVVKPSAADRQHWLDKGILPSASENKTQHTHACYLDTTNGKKIYHGRIVCRLQAETPMFIGGRHRENTEPTEILPFTLGGKPAIPATSLRGMLSSIAEAASNSSLRVLEDKTLSYRKSMRANRNEDKPLSALGMIKKIETGDKVEYRLLPLTLPTLVKRGQYYILPEEYQTMFPDGRAKLKVYLNSNYTASDGTNQDFLKGKKSWRLPHGEIYYMKLCQDFSLQNGQLTFDSQNQNMLHFPKNRNNFVVGQRSIDNTPPMTKAEWRTNHQTGVPGMLRIMQASGRNFPTGRYHEIFIPVPTKKDCKQLYPVDEKAVERFLDLAGEQTKSQQNEKNLKQYQILPYHPVGTKRNTDPETNDRYMDLNSGDIVYFRPDATGTKVEEISFSSIWRDRVEDDNHNRAGVHAFFGNIDKELLPFNPKRAEISPAELLFGFVEERERGKVDDGQAPAFAGKVRVSFGRLSSEKKPDTIFQDQVTLKALSSPKPPSPALYFTGNGNGSIAKPDLTLSRHSPQGRKFYLHAWQEENEIIKFLSNGKKTSPTVINGLYPWESKSNLSRQLPDKHAKLKSAITPIKKGTVFYFHVDFDNLSEWELGLLCYALQPGKEFRHKLGMGKPIGLGSIHIEPAGLYLVHRGNRYSLDGVPDNSRYNGGIWQSEDKRLQEWQELYPRESTAAASSAAASPADFALKFSGTMTPSVQQALKRLGDPDNVVAPVHYPQVEGADLEEETYKWFVANDVGSQTKVGNRTTCTEEAARKSMLPLAGRGPLPRLKRYKWCP

SEQ ID NO: 26 - protein, 1225 aa, Desulfobulbus sp.
MAAVQDRWTLMDQQGNELKRFRITAELETASSLHIGASETVEHDLIKNDDGTPVQINALITGAGGLPIIPGSTIKGRFLARLRERGVDSALLETLFGKGHDRETEDQGRGGRAEFHDAPLCHRLSGARHFPYWRPERQIWVKAQTAVDRHRGTALRRSLRYTEMVPPGVRFRLTITGCMTDAEADVLFALLEDLGDPRQACSFGGAGADGNGTMRLFGRPEVYCLDRSGILGWLASFEKGGNGGMAMTAAALLQADTVQRRADKVRQAWQPPDVGPRLHVELRFSGPFLVNDPSRNTPDITQAPDMVPLVDEDGNPMLPASSFRGALRAQAERIIRTLGGRCCDTSSPCRPLGSSDKVGELCLACQVFGAPGWGTTLHIQGFTCTSVFRREQEQTFVAIDRFHGGCKEGALYTIRHAESPRFEGHLVIDPRMPAWGRGLLALVFRDLREGDITFGLGAGKGYGVVDAAVVQDMAELEPYVEAFREQCRQHQGMADCHSAPSPQPLRDHDLAEIPPAEEAPGETFLNPYHFVPIREPDTGSWLARDELDSSCCHSHGFYRQQVDDRPLYHGRLTCLLETETPLFIGATGDSSVPSRIENYRLGNRIAIPAASLRGMLSSLAEAASNSAMRVLHQGILSYRKKAKNALREIGMIVLRDGKRFILPLVPLMEVTKLRHAYTDPAMKHFLDDKNSWSPRCNRVYYLGRDGNQIPAETRGAGMRPGILRLLGREGRHDALQNKKHEYFIEIPERYVDQDHCFDYRMFIRDRARNGTLVPISPVAWERYHCLAEERTLSQKNDPELREDKACASLKWLPFHPKGRVRERDPENDVCHLSLRHGDLVFYAEQNRVVSEISFSAIWRSRVETSDSYQAVTVDCFVPKELRPFNRDRRAISPAELLFGFVELDESEHSTEKSRYEQMAFAGKVRLSAGLPVEDVEDSALLEPKPIVLKALSSPKPPSPPFYFVMRDGSGAYIAKKDLSPDRHRIKGRKHYLHGLRQRGNPDRVQSLDRYGHATETAANPPWETCHPEERPQIKVRVQPVRRKTKFFFHLDFSNLSRWELGLLCYVLRPTACFRHKLGMGKPLGLGSVRIDIASLQLIDRVRRYGTDDLTAGRYNMGGHFNASCLDLLPQQDSPAPDDSGAAPDPGTLRQDFVKTMDETVFRALDLLGNPAHVQRPVHYPQVREMDIEDQTFLWFVDNDKQWKDALQPLTSSSTQLPPLTRRNKR

SEQ ID NO: 27 - protein, 1234 aa, unidentified
  REGION 1..1234: note = source = /note="Description of Unknown: Gammaproteobacteria bacterium sequence"
MTTVKEKSWAFTGLKRWKIITTLETQTPLHIGSGEVAEIEINDSQGDRRQVQANAIIRGKDDKPIIPGSTLKGKLRSHFESCLDHSKALERVFGKEYQSDEEQGRGGLAEFHDAVCSYVAPGNSYYPNWNEARNTYIEASTTIDCHTGTAADATLHYNECVPPGTRFLVTVTGAMSDKDAALIVAALQAFGDETNPIHLGAEEANGKGRMGLFGNVEVSCLDHDDIIAWISQGSDARMATDKFKPLGKEKVNDLAKNITTPTATGGAQRQHFGIELKFDGPFLVNDPSKYSKGDGDQPAVHQPLTDRSNNPILPARSFRGAIRAQAERIIRTMGGACCDTQSPCQNSGQLCIACQMFGTTGWKTTLSISDFTYDGEYRPAKTQQFVAIDRFHGGGKDGALFSIKYFERPVLKGGISLKLRNQNADELSWRKGLLALLFRDLQEGDITFGFGANKGYGGVEEACITNADVISTADIEAFRAKCHANHADSWCSPVSKPTNRDDKSSLPSINPATGAGHAFHNPYHFIPIKAPDTSTWLDKHKLATPGSPHSHAYYRSCSDDDKPLHHGRITCKLTAETPLFVGSGDAENQLTDSEAKLKEHYQLNNKLAIPATSLRGLISSLAEAASNSALRVLDNGVLSYRKPASRALRKIGILFKREEQWRLVQMEGNLANAIKLKSAYTNQKMMDFLANKQSWSPEHNVVYYLSADFRPGDVPQETYLAGRICGILRILGGKDGDRKNELENKKHELFIRVDEQYVDTEINRFDYEEYVRQGGIPVSPSAVERYTELADQRSLSQKNSRDLKGDNNCCSDKWLPFHLKGAARYKKEKACLLPLREYDLVYFDSDGTQVTEISFSAIWRDRVADKVHAFFPEELRPFNQKRKWISPAELLFGFVELNDNKDERDHAQAFTGKVRVAAGVLSPDDSIRQGDLQEHEPIMLKALSSPKLPSPALYFKQKSGDHRYIAKPDLKKASHQAQGRKIYLHALRDQKDDVQKLNTKGQPANGNGAHLPWKTADEDERPQLKVRIRPLKPGTSFYFHLDYNNLTEWELGLLCYVLQPSETFRHKLGMGKPIGLGTVKIEIATLQTIDRQKRYREAGANEHRHNGSNWVNESLRDELERLPGTVELSPDRQPEAKLRPDELRQSFIATMDNDIYRAIELLGDPHNIKYPVHYPQVRNKSIEQENFKWFVANDSGSGDQRKGTGIDAKEEPMRSIDQISTTIPTLNRYEWNGD

SEQ ID NO: 28 - protein, 1258 aa, Desulfobulbus sp.
MARNNKQYHFIPRWEIKVNLTTRSFLHIGCDEFTDRPGLEIEQKDGSKVKAEINAFIKDSNGKPYLPGSTIKGNIRKWLETNKKADEETCKLFNTLLGFTVKMQDEGCGGSAEFHNAVISSPLEDGNNFPYWDVDLQTSVETSTVIDRVTGTVVDGRLFSTEVVPPEVSFTLIITGAMTEQQVSLLTAVIKDGFAEDCPTPITIGADSGNGFGRFRFDSIHMKCLGTGEVLNWLEDGSQDMAATAMRSLSPDDIEQHIIKGRNYLKSPSVSDTVTIEFGFAGPFLVNDPSRKKRKEDIDHQPLRDSAGNARLPAKSIRGAMRSQAEKIIRTLGGWCCDPVNPCPSVFSVVEINDRLCLACRVFGATGWKSRISIQKVEYKGTAESTRQETVQDFVAIDRFHGGGKETAKFDASFSWRPQYSILMHIPSDLEGWAKGLLALTFRDFKEGDIFLGYGRSKGYGRVDSDSVKPGIDTMLTESNLELFRRKCDDNPGEYPCKTRQPPNLVQPVERNNLTEAADEGSFHNPYHFIPTPKPMIESWLAKEDFDETMHDSHALYRDVDENEEPLYHGKISCTLTTETPVFVGGKHDPRNDTEPQQVDHYTENGEIAIPATTLRGLLSSLSEAASNSSMRVLDDGMMSYRQPVGSGSLSAIGMVVIRDGKKFIYPLALPIFGERDKLPQEYHIMFPYTQKAPLKVYLERAYLAGNMKSFLDKQNSWNLLNEKIFYLPVPEFSFSRVHTMGAENRDVLKISRRGNLILGARLPVNLCPRSKEKALPGDIPGILRILGKEGRDGEVPVGKKHELFIPVSDGFASNPRSFIDNLTSKELFKIPDEVVDRFEELADDRTTQQIKHPGNVKNNNQWLPFHLKGCTRNDGLTGKDEKRLRVQEGDLMYFRPSPQSPQVAEISFSAVWRGRVNKTVHNYFPPELVQFNKNREKISPAELLFGFVQQDKHEKSLSFAGKVVLSSGKQLRETESVSRENEVTLKILASPKLPSPSLYFKRENYIEGGNYIAKNEMNNSSNIKPRGRKQYLHALSNSEDPKGVQKISRTGSVDDGGNYPWQSMNNDNIKQKVCIRPVSKDGCFTFEMEFENCTEWELGMLLYALRPSQQYRHKIGMGKSIGLGTVRIDINNLQFIHRKNRYNAGIIDVPRYNYEAGHDMDYFHNKFADTIMPEIKNSIELLGDPRNVRFPVHYPQVHGADIEDKTYQWFVANDSGTNNGQNGAAYKKNKAEESSLTELDEISNTIPGLERHEWLGR

SEQ ID NO: 29 - protein, 1235 aa, unidentified
  REGION 1..1235: note = source = /note="Description of Unknown: Methylococcaceae bacterium sequence"
MALKTWTLNGEERWHISVVLETVTPLHIGSGEFCYRPELTNADQKPVDINACIKGANNLPIIPGSTVKGKFNAWLTARQVDTPLLEAIFGKGHNPDDDDQGSGGKVEFHDAWISTKIKDTSTWPYWQVATQTFIDAATAIDRHSRTALDASLHYTECVPPGVQFTLNITGVMQEHEAALIIAALDRFDQHDDQPYFGAGDANGQGQLILVGHLAVKVMGKTEITEWLAHFNNKASDMAMSHARSLGAEDIAGLIKLGQTLLKPVPPTVSLGIQLQFAGPFLVNDPYAVKKLEADPKTKIDHYPLLDNHKKPRLPSASIRGVLRSQAERIIRSLGVHCCDTRDPCPSLYKHQDLSQLCLACQIFGAAGWKSVINISDFTCVDANELKTQEFIAIDRFHGGGKDGAKFNAKHSERPYFQGRITLSPRMANHQLDWGKGLLALVIRDLQEGDLSFGFGANKGYGALESVLITGIDQLQTDAIEAFRRLCVTQAAPQAFITPTSAVVIGDKAPLVVTDKKLPDNSFHNPYHFIPINSPDTRHWLPTETDLAESHHSHAYYRQQPELFHGQLICRLYTETPTFIGASKKDDTLPAELDNYRLNGQLAIPATSLRGMISSLAEAASNSAMRVLDNGLLSYRKDASLALSKIGITFINRQGQWQLIPMEKIKLKNAYSAENMRLFVEQSHSWSPDYNTVYYFSEKAGAFDVPQRTPKPGWQPGILRLLGKEGRSQELENKKHEWFIPVPENYIDKQLNAFKYQEYLKDNSSKAIDIPAPVLNRYNELAYQRTLSQKKDTELVADGDSPAWLPFHLKGQQRQPQMVGKHLVYTLPMTEYSLVYYAATNKVATEISYSSIWRGRVQDDADQAATVNHFIPDDLLPFNPKRTSLSPAELLFGFTELDPDKHSNDPTRSFAGKVRIGAATLAAYPSNDSDLLAPEHITLKALSSPKLPSPALYFRTLQGNNSNVYIPKHELNPNHHTAKGRKYYLHATRTPDQKRILKLSDQGHPPQNNAVKLPWLSHQETKNLQLKVKIKPIKPKQSFYFQVDFNNLTAWELGLLCYALRPTIDFRHRIGMGKPLGLGSVKIDILALQTLDRQKRYAQDSQDSARYNQHRWVNSSVTDMLAQAGYDVIEPTANPLVPKDLKTLFSQTMAANIDRALTLLGEPQHVKQPVHYPQVRDTAIQVRDTAIEEESYQWFVANDNLSDNSSAAKQTLHDITETSEGLPTLIRHQKKKETQP

SEQ ID NO: 30 - protein, 1276 aa, unidentified
  REGION 1..1276: note = source = /note="Description of Unknown: Gammaproteobacteria bacterium sequence"
MSDTQKQAIHENKWHFRGIKRWEISAYLKTLSPLHIGDGGTIPVTIKDTQGKNREVEVNSVITGKAALPIIPGSTIKGRLRHYFSKHFSDKALLNKVFGEESDATDDDQGRGGLAEFHDAKWNPEKNRNLQGRYPYWNNTRKTYIEVSTAINRHTGAAKDKSLHHTECVPPGTVFEIKITGSMDDRCAALVVAALEAIQTTGSRIFLGAEDANGNGRIGLTGKITVKQMDQAHIIQWLQKDSTTCVASFSNVKAENETQVKQMVQRHIAPKLNSVVSAAGPSYDITLHFDGPFVVNDSDKCKAEDTPDIYPLEEKNGVPAFPVRSFRGAIRSQAERIIRTIGGQCCDGSINNTCKNPKNLCIACEMFGSTGWKTSIEMDPFLCVDRELKPFIIQEFVAIDRFHGGGKDEAKFNAAHYQAPVFKGKVRVSQRVGNDISWRKGLLALIFRDLKEGDIYFGFGTNKGYGAVKKAEINPDGNASDFSESDIEAFINKCREKKGLYNCNPIKKPGKTKVSKNLPPAIVPLDRTDSKFYNPYHFIPVKKPNTSSWAEKTAFGTADSPHSHGFYRKQTNEQQPLYSGRLICMLTSETPFFIGAQAESDPTENENQASLRHPYQLDGEPAIPSTSLRGLISTMTEAAANCAMRVLDSEIISYRKPMNPSHILSALGMVTKRGEDFWLIPLAMPALSLNDEEHNYKLDKRYRTMFPDGLAKLKVYLEKAYSNNVMKTFLNNENTWTLAQSKIHYLPLTPIQMQNGGINSYYNNLRTPSRSNNFLIGQTVAHGNGIPASGPGAGMVPGILRILGKEHRQNDLPQNKKHELFIPVPDAFVADPKTFLDTATAFLIPRNVIDAFEKIAEKQTQSQKQDKLKHDEERLPFHLKGTRREQNHTLQIKTGDLVYFRPNAKGDEVEEIAFSSIWRGKTSGTTADFFPDKELLPFNRNRSRVSPAELLFGFTENNPKEMKIDRGLAFAGKIRISAGTLSDKFSDTTESDLFEPETTLKALSSPKPPSPALYFKEKKSGTQYIKKQDLNPGKHEIQGRKIYLHALRNENNQNVQRITSQGKFDNAANRTQPWVSQNEERNHLKTKCKPLKSGLNFFFHIDFNNLTQWELGLLCYALRPCETFRHKIGMGKPIGLGTVKIDIAVLQTIDRYARYTDTTQDSERYNQGAWISQELQNEIPNQYKGKGISNKKGMLSPEDCRKVFMETMDADIQRAIELLGDPGNVTSPVHYPQLDRKNIETKNYEWFKQNEIEQQVLKPITKNTTHLTPFARWEQG

SEQ ID NO: 31 - protein, 1221 aa, unidentified
  REGION 1..1221: note = source = /note="Description of Unknown: Methylobacter tundripaludum sequence"
MNLPTWKLNNEKRWHISIVLTTATPLHIGSGEFCEHDDVKNNDGEPVKINACIKGSKGRPIIPGSTIKGKLYEWLKTRNTEENLLEKLFGKGHNSVSQDQGRGGKAEFHDAEIIEPLTGSQPWPYWREEHQAFIAASTAIDRHKQVALQQSLHYMETVPAGIRFKFTFTGVMRDEEAALLIAALDSFDKNQNQPCFGVDRANAYGRMELHGHLHVKVMGATEISSWLNSFSENDKKMAMESARNLEQQEINTLIKQGNALFKASCDEVKLGLTLKFKGPFLVNDPYAVKILSSNENAKTDHYPLLDKNRNPYLPVSSFRGVLRSQAERIIRTLGGKCCSTDDPCKPIFDKGDLSKLCLACQIFGASGWKTVINIHDFKAINKSKKTKQDFVAIDRFHGGGKDGAKFDATHFERPEFEGAISFSPRMANNDLDWGKGLLALVLRDMQEGDMTFGYGANKGYGGLESASITGIEQITSDIQAFRDKCVASPQTWLCDEAVKPANQQDKIPPAGIQVANSGFHNPYQFIPSKDPDTGHWLPVLGLNADSHHSHAFYRDQTDNGEKLYHGRLICCLNTETPIFIGADKKKDTEPAEINNYRLNGELAIPATSLRGMISSLAEAASNSAMRVLDNGLLSYRKTADDALRKVGMVIYVDNKSFIIKLNDAIKLKQTYTPGNMKDFIEKSNSWSPEHNTVYYLDNNQIPQESYMNGMKPGILRILGKEGREQELENKLHELFIPVPLEYVDTENNKFDYQAYKKAFLYRAIEIPEPVLKRYSELADQRTMSQKSNKELKKDDTCQSVGWLPFHLKGTKRQLDDKHKVGKLQIDEYDLIYYEASGKEVTEVAFSSIWRGRVETNSSQANKVYSFIPGELLPFNESRKKVSPAELLFGFTQINKDGSKADDKAQAFAGKVRISAGTISEYPESEANLLEQEVTLKALSTPKLPSPALYFRTINGNGSAYISKQELEPSKHLAKGRKYYLHALRTGDNKVQKLGSQGETANGGDSKLPWVTHNPDERPQLKVKIKPIKAEFIFSLDFNNLTEWELGLLCYALRPTDSFRHRIGMGKPLGLGSVKIDIMALQTINRQQRYAQDGLEENRFNRHNWVNPPHQPRLDKAGYSISLSSTPLNPEILRATFTKTMNADIYRTLELLGNPQNVKRPVHYPQVENHNIEQENYKWFVANDQGSGKGRNKIDPAEKALKILTENSDCLPTLSRLDWRDE

SEQ ID NO: 32 - protein, 1444 aa, Nitrosomonas sp.
MNNKGSNMTDTVKSGRWIITGQFQLVTPMHIGTGLDEEMDKQSGESVDKKQNNSWIQAIALDLNKKPYIPGASIKGALKALARRYYCASNLNIFGDTIDTKDGDNKRKSVTVAGQAEFLNAWYAADQEDKPFDTITRVAIDRVTGTAEDRKLFNTRRVNPGVCFNYKIIIQNACETEIQYLLDLLRKAAKDPSFSLGAGANQSQGKVRCLSSCVRYFGKQEMHDWFRAIQNGKQEHWQIFAKPSNIKYADLERPDIIANSLNLPLTLDFHTPFLVKASKKKDEAKNEADAKPRTNHQGQVILPASSLRGRLRAQAEKILRTMGQDIPQGHAAPAYDGIAHRDLISLLFGTAGWKGIVCASDLIHSIPEYALQFNGVRETISDLSDTVKSCIIVDLVKTSTAAEKTEEQLHIRIVDSAGSLIVHKSENSSWANDTFRDASVKDNFKARLKEIADPQDLSDALRADIKKRAFQLATLTRHEMVAIDRFTGGGKEGAKFNVDYIECPTLTGAIYLDLHRLKQAQLKNDEDALKPALGLISLLLRDLAEGDIAFGFGANKGYGQCREHAVLDNWEERLKKIGAGLTIDGALQALRDTVALEPPAEFPPEIEKTTDDNQPEAPDFNLKPASNGFHNPYHFIPLNNPKIGDWPEAKAETLKANREGHDQYHTGKFSGRIVCSLTTQTPLFIGAETKPSTSDREPSEARPFKLNGKHAIPATSLRGMLSSLFESVSNSNFRVLHPEHYSVRKSLDDYVALSAMGRIVDDQGELKLQPLTLPTLFGNRNNVPAKWEKIFGTPSEDDFLRIYFDDIPSKFSSNKRYFYNCKATELKDFIKSDKYFIGKRTPTVFPKSSTEKSHLESLEFIDVEKFKKAVENLEITPGNNPYIHGWVRNLKDEFREDIPDNVKHHVFLPDTTKRVSPLEIPPHVKKRFHELADLALAGLHLKQGETIASPYKILPYTPIGRNKLENHIHRVPNDLTCYMTRLKKGDLVFFDVDNDGQITEISFSSIWRAGIGTKNKLQTTADLLSQRDPNLVQLGMGVRTKNTDRFKLSPAERLFGVVEHRDDDNTTVENVNQPNDKAQAFAFAGKVRIGFGLPDKKTTVNGVSPVTLKELSSPKPPSPAFYLKRKNNDDFVSKKVAAECSETMTLRGRKCYLHAWREQNGNVMKLDAIGVNSGGSTCKPPWKTHKPAANDQKEFEEDKNKFITSRQVKIAPISENTPFYFEIDFNNLDATELAQLCATLQPAPKFEHRLGMGKPLGLGSVKIEPVGLFLINRHQRYTTDSTNCDRYHYAWLKGEHAAWDWPEYFRQNVVTADCTQTFNDTFDKLVQNGLAGTDADIKHALQLLGDPQYIGVPVHYPIAGNSTLENKHFEWFGNNDKASVLRQKAQANSKNHHYQPKQQATPEEPQYLHTITKDSKQISLLKKNKIEDIENRDQQKHRYSNHRR

SEQ ID NO: 33 - protein, 385 aa, synthetic construct
  REGION 1..385: note = source = /note="Description of Artificial Sequence: Synthetic polypeptide"
QLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLT

SEQ ID NO: 34 - protein, 336 aa, synthetic construct
  REGION 1..336: note = source = /note="Description of Artificial Sequence: Synthetic polypeptide"
SLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGQGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWISKPQEEKNF

SEQ ID NO: 35 - protein, 340 aa, synthetic construct
  REGION 1..340: note = source = /note="Description of Artificial Sequence: Synthetic polypeptide"
SVGTGNRCLTGDHLSLEGNSVNDSHAEMITRRGFLRYLYRHLLEYDAEVPNDLFEKGERSICRIKTNITFHLYISTAPCGDGALFSPRDTDSSNAKMEEENKHIHNPTFSSSVQGLLRTKVEGGQGTIPIDADFTEQTWDGIQRGERLRTMSCSDKICRWNVVGLQGALLSHFIEPIYLDSLTLGYLYDHGHLARAVCCRIERGEASVNQLLPEGYRLNHPWLGRVTACDPPRETQKTKSLSINWCYDDEKSEVLDGTAGICYTAIEKNLFSRLTKHNLYEEFKRVCRKFDRNDLLTAPSYNKAKMMATPFQTAKNVMLKKLKENNCGTWVSKPIEEEMF

SEQ ID NO: 36 - protein, 340 aa, synthetic construct
  REGION 1..340: note = source = /note="Description of Artificial Sequence: Synthetic polypeptide"
SVGTGNRCLTGDRLSLEGNSVNDSHAEMVTRRGFLRYLYKHLLEYDPEKPHDLFEKGERSLCRIKTNITFHLYISTAPCGDGALFSPRDTDSSNVKVDEENKHVHNPTFSSSVQGLLRTKVEGGQGTIPIDADFTEQTWDGIQRGERLRTMSCSDKICRWNVVGLQGALLSHFVEPIYLESLTLGYLYDHGHLARAVCCRIERGEASVNQLLPEGYRLNHPWLGRVTACDPPRETQKTKSLSINWCYDDEKSEVLDGTAGICYTAIEKNLFSRLTKHSLYEEFKKVCQKFEREDLLNVTSYNKAKMMAIPFQTAKNVMLKKLKENNCGTWVSKPIEEEMF

SEQ ID NO: 37 - protein, 328 aa, synthetic construct
  REGION 1..328: note = source = /note="Description of Artificial Sequence: Synthetic polypeptide"
GIGTGTKCINGEHMSDRGFGVNDCHAEIIARRCFLRYIYDQLELHLSDNSDVRNSSIFELRDKGGYQLKENIQFHLYISTAPCGDARIFSPHGQDVETGDRHPNRKARGQLRTKIESGQGTIPVRTSGVIQTWDGVLEGERLLTMSCSDKIARWNVLGIQGSLLSHFMNPIYLESIILGSLYHSDHLSRAMYSRISIIENLPEPFHLNRPFLSGISSPESRQPGKAPNFGINWRKEDETFEVINAMTGRVEGGSVSRICKQALFGRFMSLYGKLSSLTGQSVTTRPTHYSDAKAAVMEYQLAKQCVFQAFQKAGLGNWVQKPIEQDQF

SEQ ID NO: 38 - protein, 328 aa, synthetic construct
  REGION 1..328: note = source = /note="Description of Artificial Sequence: Synthetic polypeptide"
GIGTGTKCINGEYMNDRGFAVNDCHAEIIARRCFLRFIYDQLEMHLSEDPEVRGQSVFELRDGGGYKLKPNIHFHLYISTAPCGDARIFSPHGQDVETGDRHPNRKARGQLRTKIESGQGTIPVRSSGFIQTWDGVLEGERLLTMSCSDKIARWNVLGIQGALLCHFMHPIYLESIILGSLYHSDHLSRAVYCRIASIENLPDLFQLNRPFLSGISSPESRQPGKAPNFGINWRRNDDTFEVINAMTGRVEGGNMSRICKQALFDRFMNLYGRLSSLTGQSVTTRPTLYSEAKAAVMEYQLAKQCVFQAFQKAGLGNWVQKPIEQDQF

SEQ ID NO: 39 - protein, 328 aa, synthetic construct
  REGION 1..328: note = source = /note="Description of Artificial Sequence: Synthetic polypeptide"
GIGTGTKCINGEYMNDRGFAVNDCHAEIIARRCFLRFIYDQLELHLSDNAEVRGQSIFELRDAGGYKLKPNIQFHLYISTAPCGDARIFSPHGQDVETGDRHPNRKARGQLRTKIESGQGTIPVRSSGFIQTWDGVLEGERLLTMSCSDKIARWNVLGVQGALLCHFMHPIYLESIILGSLYHSDHLSRAVYCRIAAIENLPDLFRLNRPFLSGISSPESRQPGKAPNFGINWRRNDDSFEVINAMTGRVEGGSMSRICKQALFDRFMNLYGKLSSLTGQSVTTRPALYSEAKATVMEYQLAKQCVFQAFQKAGLGNWVQKPIEQDQF

SEQ ID NO: 40 - protein, 236 aa, unidentified
  REGION 1..236: note = source = /note="Description of Unknown: APOBEC1 sequence"
MTSEKGPSTGDPTLRRRIEPWEFDVFYDPRELRKEACLLYEIKWGMSRKIWRSSGKNTTNHVEVNFIKKFTSERDFHPSMSCSITWFLSWSPCWECSQAIREFLSRHPGVTLVIYVARLFWHMDQQNRQGLRDLVNSGVTIQIMRASEYYHCWRNFVNYPPGDEAHWPQYPPLWMMLYALELHCIILSLPPCLKISRRWQNHLTFFRLHLQNCHYQTIPPHILLATGLIHPSVAWR

SEQ ID NO: 41 - protein, 224 aa, unidentified
  REGION 1..224: note = source = /note="Description of Unknown: APOBEC2 sequence"
MAQKEEAAVATEAASQNGEDLENLDDPEKLKELIELPPFEIVTGERLPANFFKFQFRNVEYSSGRNKTFLCYVVEAQGKGGQVQASRGYLEDEHAAAHAEEAFFNTILPAFDPALRYNVTWYVSSSPCAACADRIIKTLSKTKNLRLLILVGRLFMWEEPEIQAALKKLKEAGCKLRIMKPQDFEYVWQNFVEQEEGESKAFQPWEDIQENFLYYEEKLADILK

SEQ ID NO: 42 - protein, 199 aa, unidentified
  REGION 1..199: note = source = /note="Description of Unknown: APOBEC3A sequence"
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 43 - protein, 251 aa, unidentified [sequence omitted]

SEQ ID NO: 44 - protein, 190 aa, unidentified
  REGION 1..190: note = source = /note="Description of Unknown: APOBEC3C sequence"
MNPQIRNPMKAMYPGTFYFQFKNLWEANDRNETWLCFTVEGIKRRSVVSWKTGVFRNQVDSETHCHAERCFLSWFCDDILSPNTKYQVTWYTSWSPCPDCAGEVAEFLARHSNVNLTIFTARLYYFQYPCYQEGLRSLSQEGVAVEIMDYEDFKYCWENFVYNDNEPFKPWKGLKTNFRLLKRRLRESLQ

SEQ ID NO: 45 - protein, 455 aa, unidentified
  REGION 1..455: note = source = /note="Description of Unknown: APOBEC3D sequence"
MNPQIRNPMERMYRDTFYDNFENEPILYGRSYTWLCYEVKIKRGRSNLLWDTGVFRGPVLPKRQSNHRQEVYFRFENHAEMCFLSWFCGNRLPANRRFQITWFVSWNPCLPCVVKVTKFLAEHPNVTLTISAARLYYYRDRDWRWVLLRLHKAGARVKIMDYEGERCRGQGSMTGRNSLRDGWICNAMAGGVPGQPAGVGLALIATDSQETRPGRAGPGSGESLSASHLFISDFAYCWENFVCNEGQPFMPWYKFDDNYASLHRTLKEILRNPMEAMYPHIFYFHFKNLLKACGRNESWLCFTMEVTKHHSAVFRKRGVFRNQVDPETHCHAERCFLSWFCDDILSPNTNYEVTWYTSWSPCPECAGEVAEFLARHSNVNLTIFTARLCYFWDTDYQEGLCSLSQEGASVKIMGYKDFVSCWKNFVYSDDEPFKPWKGLQTNFRLLKRRLREILQ

SEQ ID NO: 46 - protein, 373 aa, unidentified
  REGION 1..373: note = source = /note="Description of Unknown: APOBEC3F sequence"
MKPHFRNTVERMYRDTFSYNFYNRPILSRRNTVWLCYEVKTKGPSRPRLDAKIFRGQVYSQPEHHAEMCFLSWFCGNQLPAYKCFQITWFVSWTPCPDCVAKLAEFLAEHPNVTLTISAARLYYYWERDYRRALCRLSQAGARVKIMDDEEFAYCWENFVYSEGQPFMPWYKFDDNYAFLHRTLKEILRNPMEAMYPHIFYFHFKNLRKAYGRNESWLCFTMEVVKHHSPVSWKRGVFRNQVDPETHCHAERCFLSWFCDDILSPNTNYEVTWYTSWSPCPECAGEVAEFLARHSNVNLTIFTARLYYFWDTDYQEGLRSLSQEGASVEIMGYKDFKYCWENFVYNDDEPFKPWKGLKYNFLFLDSKLQEILE

SEQ ID NO: 47 - protein, 325 aa, unidentified
  REGION 1..325: note = source = /note="Description of Unknown: APOBEC3G sequence"
MELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQEN

SEQ ID NO: 48 - protein, 154 aa, unidentified [sequence omitted]

SEQ ID NO: 49 - protein, 367 aa, unidentified
  REGION 1..367: note = source = /note="Description of Unknown: APOBEC4 sequence"
MEPIYEEYLANHGTIVKPYYWLSFSLDCSNCPYHIRTGEEARVSLTEFCQIFGFPYGTTFPQTKHLTFYELKTSSGSLVQKGHASSCTGNYIHPESMLFEMNGYLDSAIYNNDSIRHIILYSNNSPCNEANHCCISKMYNFLITYPGITLSIYFSQLYHTEMDFPASAWNREALRSLASLWPRVVLSPISGGIWHSVLHSFISGVSGSHVFQPILTGRALADRHNAYEINAITGVKPYFTDVLLQTKRNPNTKAQEALESYPLNNAFPGQFFQMPSGQLQPNLPPDLRAPVVFVLVPLRDLPPMHMGQNPNKPRNIVRHLNMPQMSFQETKDLGRLPTGRSVEIVEITEQFASSKEADEKKKKKGKK

SEQ ID NO: 50 - protein, 180 aa, unidentified
  REGION 1..180: note = source = /note="Description of Unknown: Activation-induced cytidine deaminase (AID) sequence"
MDSLLMNRKFLYQFKNVRWAKGRRETYLCYVVKRRDSATSFSLDFGYLRNKNGCHVELLFLRYISDWDLDPGRCYRVTWFTSWSPCYDCARHVADFLRGNPNLSLRIFTARLYFCEDRKAEPEGLRRLHRAGVQIAIMTFKDYFYCWNTFVENHERTFKAWEGLHENSVRLSRQLRRILL

SEQ ID NO: 51 - DNA, 31 bp, synthetic construct
  misc_feature 1..31: note = source = /note="Description of Artificial Sequence: Synthetic oligonucleotide"
  source 1..31: mol_type = other DNA
ctttgttgtcttcgacatgggtaatcctcat

SEQ ID NO: 52 - DNA, 31 bp, synthetic construct
  misc_feature 1..31: note = source = /note="Description of Artificial Sequence: Synthetic oligonucleotide"
  source 1..31: mol_type = other DNA
taaagagttgaacttctttgttgtcttcgac

SEQ ID NO: 53 - DNA, 31 bp, synthetic construct
  misc_feature 1..31: note = source = /note="Description of Artificial Sequence: Synthetic oligonucleotide"
  source 1..31: mol_type = other DNA
gtaccttcgtacggacgaccttcaccttcac

SEQ ID NO: 54 - DNA, 31 bp, synthetic construct
  misc_feature 1..31: note = source = /note="Description of Artificial Sequence: Synthetic oligonucleotide"
  source 1..31: mol_type = other DNA
caggatgtcccaagcgaacggcagcggacca

SEQ ID NO: 55 - DNA, 31 bp, synthetic construct
  misc_feature 1..31: note = source = /note="Description of Artificial Sequence: Synthetic oligonucleotide"
  source 1..31: mol_type = other DNA
cagggaggagtcctgggtaacggtaacaaca

SEQ ID NO: 56 - DNA, 31 bp, synthetic construct
  misc_feature 1..31: note = source = /note="Description of Artificial Sequence: Synthetic oligonucleotide"
  source 1..31: mol_type = other DNA
catgaactctttgataacgtcttcgctactc

SEQ ID NO: 57 - DNA, 31 bp, synthetic construct
  misc_feature 1..31: note = source = /note="Description of Artificial Sequence: Synthetic oligonucleotide"
  source 1..31: mol_type = other DNA
tcggaggtagcccgactcgctccggttcgta

SEQ ID NO: 58 - DNA, 31 bp, synthetic construct
  misc_feature 1..31: note = source = /note="Description of Artificial Sequence: Synthetic oligonucleotide"
  source 1..31: mol_type = other DNA
tgaaagttggcgccttttttcatacgtcgag

SEQ ID NO: 59 - RNA, 300 nt, synthetic construct
  misc_feature 1..300: note = source = /note="Description of Artificial Sequence: Synthetic polynucleotide"
  source 1..300: mol_type = other RNA
gggcgaaatcggtatttccaatgaaatcactcaggttggaaagccggttttctttgatgtcacggaaccgccaatataacaatacaggtattatagttgggttggaaagccggttttctttgatgtcacggaacattgtaaactgcatatctgtgtttgccgtctggttggaaagccggttttctttgatgtcacggaactcacgtgagtcagtcttagttctatacttctttagggttggaaagccggttttctttgatgtcacggaacaggaacttgaacaacatcgttactaacgag

SEQ ID NO: 60 - RNA, 176 nt, synthetic construct
  misc_feature 1..176: note = source = /note="Description of Artificial Sequence: Synthetic polynucleotide"
  source 1..176: mol_type = other RNA
gggggccagtgaattcgagctcggtacccggggatcctctagaaatatggattacttggtagaacagcaatctactcgacctgcaggcatgcaagcttggcgtaatcatggtcatagctgtttcctgtgtttatccgctcacaattccacacaacatacgagccggaagcataaag

SEQ ID NO: 61 - RNA, 153 nt, synthetic construct
  misc_feature 1..153: note = source = /note="Description of Artificial Sequence: Synthetic polynucleotide"
  source 1..153: mol_type = other RNA
ggggcaaactccggcatctactaatagacgccggccattcaaacatgaggattacccatgtcgaagacaacaaagaagttcaactctttatgtattgatcttcctcgcgatctttctctcgaaatttaccaatcaattgcttctgtcgctact

SEQ ID NO: 62 - RNA, 153 nt, synthetic construct
  misc_feature 1..153: note = source = /note="Description of Artificial Sequence: Synthetic polynucleotide"
  source 1..153: mol_type = other RNA
gggggccagtgaattcgagctcggtacccggggatcctctagaaatatggattacttggtagaacagcaatctactcgacctgcaggcatgcaagcttggcgtaatcatggtcatagctgtttcctgtgtttatccgctcacaattccacaca

SEQ ID NO: 63 - RNA, 150 nt, synthetic construct
  misc_feature 1..150: note = source = /note="Description of Artificial Sequence: Synthetic polynucleotide"
  source 1..150: mol_type = other RNA
ggccagtgaattcgagctcggtacccggggatcctctagaaatatggattacttgggtcgaagacaacaaagaagttcaactctttatagaacagcaatctactcgacctgcaggcatgcaagcttggcgtaatcatggtcatagctgtt

SEQ ID NO: 64 - RNA, 150 nt, synthetic construct
  misc_feature 1..150: note = source = /note="Description of Artificial Sequence: Synthetic polynucleotide"
  source 1..150: mol_type = other RNA
atgcccgccatgaagatcgagtgccgcatcaccggcaccctgaacggcgtggagttgtcgaagacaacaaagaagttcaactctttacgagctggtgggcggcggagagggcacccccgagcagggccgcatgaccaacaagatgaagag

SEQ ID NO: 65 - RNA, 150 nt, synthetic construct
  misc_feature 1..150: note = source = /note="Description of Artificial Sequence: Synthetic polynucleotide"
  source 1..150: mol_type = other RNA
attctatgtctggttttcgctcaaaaacttcccggaaatgacaacagcacggcaacgtcgaagacaacaaagaagttcaactctttagctgtgccttgggcaccatgcagtaccaaacggaacgatagtgaaaacaatcacgaatgacca

SEQ ID NO: 66 - RNA, 150 nt, synthetic construct; identical to SEQ ID NO: 65
  misc_feature 1..150: note = source = /note="Description of Artificial Sequence: Synthetic polynucleotide"
  source 1..150: mol_type = other RNA

SEQ ID NO: 67 - RNA, 150 nt, synthetic construct
  misc_feature 1..150: note = source = /note="Description of Artificial Sequence: Synthetic polynucleotide"
  source 1..150: mol_type = other RNA
gctgtgccttgggcaccatgcagtaccaaacggaacgatagtgaaaacaatcacgaatgaccagtcgaagacaacaaagaagttcaactctttaattctatgtctggttttcgctcaaaaacttcccggaaatgacaacagcacggcaac

SEQ ID NO: 68 - RNA, 173 nt, synthetic construct
  misc_feature 1..173: note = source = /note="Description of Artificial Sequence: Synthetic polynucleotide"
  source 1..173: mol_type = other RNA
ggggtttggaagccgggtcgaagtcaggcccgttaagaccgagtagattgctgttctaccaagtaatccagtttggaagccgggtcgaagtcaggcccgttaagactatgcttccggctcgtatgttgtgtggaattgtttggaagccgggtcgaagtcaggcccgttaagac

SEQ ID NO: 69 - RNA, 259 nt, synthetic construct
  misc_feature 1..259: note = source = /note="Description of Artificial Sequence: Synthetic polynucleotide"
  source 1..259: mol_type = other RNA
ggggctagctcagtcctaggtataatgctagcgctgatttaggcaaaaacggcttgaagactaaaggaaggaattaatgtcacggtacgactaaatggcaccactaattgttgccttctaagttccttcttgaagactaaaggaaggaattaatgtcacggtacgtttgtttaaccacatgaggcacaccgccctcgtgagcttgaagactaaaggaaggaattaatgtcacggtacaatctccgccccgttcgtaagc

SEQ ID NO: 70 - RNA, 400 nt, synthetic construct
  misc_feature 1..400: note = source = /note="Description of Artificial Sequence: Synthetic polynucleotide"
  source 1..400: mol_type = other RNA
atgcccgccatgaagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatcc

SEQ ID NO: 71 - RNA, 103 nt, synthetic construct
  misc_feature 1..103: note = source = /note="Description of Artificial Sequence: Synthetic polynucleotide"
  source 1..103: mol_type = other RNA
ggggttggaaagccggttttctttgatgtcacggaacctttgttgtcttcgacatgggtaatcctcatggttggaaagccggttttctttgatgtcacggaac

SEQ ID NO: 72 - RNA, 103 nt, synthetic construct
  misc_feature 1..103: note = source = /note="Description of Artificial Sequence: Synthetic polynucleotide"
  source 1..103: mol_type = other RNA
ggcgttggaaagccggttttctttgatgtcacggaacctttgttgtcttcgacatgggtaatcctcatcgttggaaagccggttttctttgatgtcacggaac

SEQ ID NO: 73 - RNA, 103 nt, synthetic construct
  misc_feature 1..103: note = source = /note="Description of Artificial Sequence: Synthetic polynucleotide"
  source 1..103: mol_type = other RNA
ggggatggaaagccggttttctttgatgtcacggaacctttgttgtcttcgacatgggtaatcctcatggatggaaagccggttttctttgatgtcacggaac

SEQ ID NO: 74 - RNA, 103 nt, synthetic construct
  misc_feature 1..103: note = source = /note="Description of Artificial Sequence: Synthetic polynucleotide"
  source 1..103: mol_type = other RNA
ggggttcgaaagccggttttctttgatgtcacggaacctttgttgtcttcgacatgggtaatcctcatggttcgaaagccggttttctttgatgtcacggaac

SEQ ID NO: 75 - RNA, 103 nt, synthetic construct
  misc_feature 1..103: note = source = /note="Description of Artificial Sequence: Synthetic polynucleotide"
  source 1..103: mol_type = other RNA
ggggttggtaagccggttttctttgatgtcacggaacctttgttgtcttcgacatgggtaatcctcatggttggtaagccggttttctttgatgtcacggaac

SEQ ID NO: 76 - RNA, 103 nt, synthetic construct
  misc_feature 1..103: note = source = /note="Description of Artificial Sequence: Synthetic polynucleotide"
  source 1..103: mol_type = other RNA
ggggttggaatgccggttttctttgatgtcacggaacctttgttgtcttcgacatgggtaatcctcatggttggaatgccggttttctttgatgtcacggaac

SEQ ID NO: 77 - RNA, 103 nt, synthetic construct
  misc_feature 1..103: note = source = /note="Description of Artificial Sequence: Synthetic polynucleotide"
  source 1..103: mol_type = other RNA
ggggttggaaaggcggttttctttgatgtcacggaacctttgttgtcttcgacatgggtaatcctcatggttggaaaggcggttttctttgatgtcacggaac

SEQ ID NO: 78 - RNA, 103 nt, synthetic construct
  misc_feature 1..103: note = source = /note="Description of Artificial Sequence: Synthetic polynucleotide"
  source 1..103: mol_type = other RNA
ggggttggaaagcccgttttctttgatgtcacggaacctttgttgtcttcgacatgggtaatcctcatggttggaaagcccgttttctttgatgtcacggaac

SEQ ID NO: 79 - RNA, 103 nt, synthetic construct
  misc_feature 1..103: note = source = /note="Description of Artificial Sequence: Synthetic polynucleotide"
  source 1..103: mol_type = other RNA
ggggttggaaagccggatttctttgatgtcacggaacctttgttgtcttcgacatgggtaatcctcatggttggaaagccggatttctttgatgtcacggaac

SEQ ID NO: 80 - RNA, 103 nt, synthetic construct
  misc_feature 1..103: note = source = /note="Description of Artificial Sequence: Synthetic polynucleotide"
  source 1..103: mol_type = other RNA
ggggttggaaagccggttatctttgatgtcacggaacctttgttgtcttcgacatgggtaatcctcatggttggaaagccggttatctttgatgtcacggaac

SEQ ID NO: 81 - RNA, 103 nt, synthetic construct
  misc_feature 1..103: note = source = /note="Description of Artificial Sequence: Synthetic polynucleotide"
  source 1..103: mol_type = other RNA
ggggttggaaagccggttttgtttgatgtcacggaacctttgttgtcttcgacatgggtaatcctcatggttggaaagccggttttgtttgatgtcacggaac

SEQ ID NO: 82 - RNA, 103 nt, synthetic construct
  misc_feature 1..103: note = source = /note="Description of Artificial Sequence: Synthetic polynucleotide"
  source 1..103: mol_type = other RNA
ggggttggaaagccggttttctatgatgtcacggaacctttgttgtcttcgacatgggtaatcctcatggttggaaagccggttttctatgatgtcacggaac

SEQ ID NO: 83 - RNA, 103 nt, synthetic construct
  misc_feature 1..103: note = source = /note="Description of Artificial Sequence: Synthetic polynucleotide"
  source 1..103: mol_type = other RNA
ggggttggaaagccggttttctttcatgtcacggaacctttgttgtcttcgacatgggtaatcctcatggttggaaagccggttttctttcatgtcacggaac

SEQ ID NO: 84 - RNA, 103 nt, synthetic construct
  misc_feature 1..103: note = source = /note="Description of Artificial Sequence: Synthetic polynucleotide"
  source 1..103: mol_type = other RNA
ggggttggaaagccggttttctttgaagtcacggaacctttgttgtcttcgacatgggtaatcctcatggttggaaagccggttttctttgaagtcacggaac

SEQ ID NO: 85 - RNA, 103 nt, synthetic construct
  misc_feature 1..103: note = source = /note="Description of Artificial Sequence: Synthetic polynucleotide"
  source 1..103: mol_type = other RNA
ggggttggaaagccggttttctttgatgacacggaacctttgttgtcttcgacatgggtaatcctcatggttggaaagccggttttctttgatgacacggaac

SEQ ID NO: 86 - RNA, 103 nt, synthetic construct
  misc_feature 1..103: note = source = /note="Description of Artificial Sequence: Synthetic polynucleotide"
  source 1..103: mol_type = other RNA
ggggttggaaagccggttttctttgatgtctcggaacctttgttgtcttcgacatgggtaatcctcatggttggaaagccggttttctttgatgtctcggaac

SEQ ID NO: 87 - RNA, 103 nt, synthetic construct
  misc_feature 1..103: note = source = /note="Description of Artificial Sequence: Synthetic polynucleotide"
  source 1..103: mol_type = other RNA
ggggttggaaagccggttttctttgatgtcaccgaacctttgttgtcttcgacatgggtaatcctcatggttggaaagccggttttctttgatgtcaccgaac

SEQ ID NO: 88 - RNA, 103 nt, synthetic construct
  misc_feature 1..103: note = source = /note="Description of Artificial Sequence: Synthetic polynucleotide"
  source 1..103: mol_type = other RNA
ggggttggaaagccggttttctttgatgtcacggtacctttgttgtcttcgacatgggtaatcctcatggttggaaagccggttttctttgatgtcacggtac

SEQ ID NO: 89 - RNA, 103 nt, synthetic construct
  misc_feature 1..103: note = source = /note="Description of Artificial Sequence: Synthetic polynucleotide"
  source 1..103: mol_type = other RNA
ggggttggaaagccggttttctttgatgtcacggtagctttgttgtcttcgacatgggtaatcctcatggttggaaagccggttttctttgatgtcacggtag

SEQ ID NO: 90 - RNA, 103 nt, synthetic construct; identical to SEQ ID NO: 71
  misc_feature 1..103: note = source = /note="Description of Artificial Sequence: Synthetic polynucleotide"
  source 1..103: mol_type = other RNA

SEQ ID NO: 91 - RNA, 103 nt, synthetic construct
  misc_feature 1..103: note = source = /note="Description of Artificial Sequence: Synthetic polynucleotide"
  source 1..103: mol_type = other RNA
ggggttggaaagggggttttctttgatgtcacggaacctttgttgtcttcgacatgggtaatcctcatggttggaaagggggttttctttgatgtcacggaac

SEQ ID NO: 92 - RNA, 103 nt, synthetic construct
  misc_feature 1..103: note = source = /note="Description of Artificial Sequence: Synthetic polynucleotide"
  source 1..103: mol_type = other RNA
ggggttggaaagccgcatttctttgatgtcacggaacctttgttgtcttcgacatgggtaatcctcatggttggaaagccgcatttctttgatgtcacggaac

SEQ ID NO: 93 - RNA, 103 nt, synthetic construct
  misc_feature 1..103: note = source = /note="Description of Artificial Sequence: Synthetic polynucleotide"
  source 1..103: mol_type = other RNA
ggggttggaaagccggttaactttgatgtcacggaacctttgttgtcttcgacatgggtaatcctcatggttggaaagccggttaactttgatgtcacggaac

SEQ ID NO: 94 - RNA, 103 nt, synthetic construct
  misc_feature 1..103: note = source = /note="Description of Artificial Sequence: Synthetic polynucleotide"
  source 1..103: mol_type = other RNA
ggggttggaaagccggttttgattgatgtcacggaacctttgttgtcttcgacatgggtaatcctcatggttggaaagccggttttgattgatgtcacggaac

SEQ ID NO: 95 - RNA, 103 nt, synthetic construct
  misc_feature 1..103: note = source = /note="Description of Artificial Sequence: Synthetic polynucleotide"
  source 1..103: mol_type = other RNA
ggggttggaaagccggttttcttacatgtcacggaacctttgttgtcttcgacatgggtaatcctcatggttggaaagccggttttcttacatgtcacggaac

SEQ ID NO: 96 - RNA, 103 nt, synthetic construct
  misc_feature 1..103: note = source = /note="Description of Artificial Sequence: Synthetic polynucleotide"
  source 1..103: mol_type = other RNA
ggggttggaaagccggttttctttgaactcacggaacctttgttgtcttcgacatgggtaatcctcatggttggaaagccggttttctttgaactcacggaac

SEQ ID NO: 97 - RNA, 103 nt, synthetic construct
  misc_feature 1..103: note = source = /note="Description of Artificial Sequence: Synthetic polynucleotide"
  source 1..103: mol_type = other RNA
ggggttggaaagccggttttctttgatgtgtcggaacctttgttgtcttcgacatgggtaatcctcatggttggaaagccggttttctttgatgtgtcggaac

SEQ ID NO: 98 - RNA, 103 nt, synthetic construct
  misc_feature 1..103: note = source = /note="Description of Artificial Sequence: Synthetic polynucleotide"
  source 1..103: mol_type = other RNA
ggggttggaaagccggttttctttgatgtcacccaacctttgttgtcttcgacatgggtaatcctcatggttggaaagccggttttctttgatgtcacccaac

SEQ ID NO: 99 - RNA, 103 nt, synthetic construct
  misc_feature 1..103: note = source = /note="Description of Artificial Sequence: Synthetic polynucleotide"
  source 1..103: mol_type = other RNA
ggggttggaaagccggttttctttgatgtcacggatgctttgttgtcttcgacatgggtaatcctcatggttggaaagccggttttctttgatgtcacggatg

SEQ ID NO: 100 - RNA, 153 nt, synthetic construct
  misc_feature 1..153: note = source = /note="Description of Artificial Sequence: Synthetic polynucleotide"
  source 1..153: mol_type = other RNA
gggaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcac

SEQ ID NO: 101 - RNA, 153 nt, synthetic construct
  misc_feature 1..153: note = source = /note="Description of Artificial Sequence: Synthetic polynucleotide"
  source 1..153: mol_type = other RNA
gggattctatgtctggttttcgctcaaaaacttcccggaaatgacaacagcacggcaacgctgtgccttgggcaccatgcagtaccaaacggaacgatagtgaaaacaatcacgaatgaccaaattgaagtcactaatgctactgaactggtt

SEQ ID NO: 102 - DNA, 34 bp, synthetic construct
  misc_feature 1..34: note = source = /note="Description of Artificial Sequence: Synthetic oligonucleotide"
  source 1..34: mol_type = other DNA
gggatgaggattacccatgtcgaagacaacaaag

SEQ ID NO: 103 - RNA, 100 nt, synthetic construct
  misc_feature 1..100: note = source = /note="Description of Artificial Sequence: Synthetic polynucleotide"
  source 1..100: mol_type = other RNA
atgcccgccatgaagatcgagtgccgcatcaccggcaccctgaacggcgtggagttcgagctggtgggcggcggagagggcacccccgagcagggccgca

SEQ ID NO: 104 - RNA, 200 nt, synthetic construct
  misc_feature 1..200: note = source = /note="Description of Artificial Sequence: Synthetic polynucleotide"
  source 1..200: mol_type = other RNA
atgcccgccatgaagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccc

SEQ ID NO: 105 - RNA, 600 nt, synthetic construct
  misc_feature 1..600: note = source = /note="Description of Artificial Sequence: Synthetic polynucleotide"
  source 1..600: mol_type = other RNA
atgcccgccatgaagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctg

SEQ ID NO: 106 - RNA, 60 nt, synthetic construct
  misc_feature 1..60: note = source = /note="Description of Artificial Sequence: Synthetic oligonucleotide"
  source 1..60: mol_type = other RNA
ccggccattcaaacatgaggattacccatgtcgaagacaacaaagaagttcaactcttta

SEQ ID NO: 107 - RNA, 175 nt, synthetic construct
  misc_feature 1..175: note = source = /note="Description of Artificial Sequence: Synthetic polynucleotide"
  source 1..175: mol_type = other RNA
  variation 76..81: note = a, c, u, g, unknown or other
ggccagtgaattcgagctcggtacccggggatcctctagaaatatggattacttggtagaacagcaatctactcgnnnnnnacctgcaggcatgcaagcttggcgtaatcatggtcatagctgtttcctgtgtttatccgctcacaattccacacaacatacgagccggaagcat

SEQ ID NO: 108 - RNA, 175 nt, synthetic construct
  misc_feature 1..175: note = source = /note="Description of Artificial Sequence: Synthetic polynucleotide"
  source 1..175: mol_type = other RNA
  variation 45..50: note = a, c, u, g, unknown or other
ggccagtgaattcgagctcggtacccggggatcctctagaaatannnnnntggattacttggtagaacagcaatctactcgacctgcaggcatgcaagcttggcgtaatcatggtcatagctgtttcctgtgtttatccgctcacaattccacacaacatacgagccggaagcat

SEQ ID NO: 109 - RNA, 34 nt, synthetic construct
  misc_feature 1..34: note = source = /note="Description of Artificial Sequence: Synthetic oligonucleotide"
  source 1..34: mol_type = other RNA
gggatgaggattacccatgtcgaagacaacaaag

SEQ ID NO: 110 - DNA, 100 bp, synthetic construct
  misc_feature 1..100: note = source = /note="Description of Artificial Sequence: Synthetic polynucleotide"
  source 1..100: mol_type = other DNA
ccggcatctactaatagacgccggccattcaaacatgaggattacccatgtcgaagacaacaaagaagttcaactctttatgtattgatcttcctcgcga

SEQ ID NO: 111 - DNA, 35 bp, synthetic construct
  misc_feature 1..35: note = source = /note="Description of Artificial Sequence: Synthetic oligonucleotide"
  source 1..35: mol_type = other DNA
ggttggaaagccggttttctttgatgtcacggaac

SEQ ID NO: 112 - DNA, 31 bp, synthetic construct
  misc_feature 1..31: note = source = /note="Description of Artificial Sequence: Synthetic oligonucleotide"
  source 1..31: mol_type = other DNA
cgagtagattgctgttctaccaagtaatcca

SEQ ID NO: 113 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 114 - DNA, 31 bp, synthetic construct
  misc_feature 1..31: note = source = /note="Description of Artificial Sequence: Synthetic oligonucleotide"
  source 1..31: mol_type = other DNA
tatgcttccggctcgtatgttgtgtggaatt

SEQ ID NO: 115 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 116 - DNA, 31 bp, synthetic construct
  misc_feature 1..31: note = source = /note="Description of Artificial Sequence: Synthetic oligonucleotide"
  source 1..31: mol_type = other DNA
aacacaggaaacagctatgaccatgattacg

SEQ ID NO: 117 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 118 - DNA, 31 bp, synthetic construct
  misc_feature 1..31: note = source = /note="Description of Artificial Sequence: Synthetic oligonucleotide"
  source 1..31: mol_type = other DNA
aattgtgagcggataaacacaggaaacagct

SEQ ID NO: 119 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 120 - DNA, 31 bp, synthetic construct
  misc_feature 1..31: note = source = /note="Description of Artificial Sequence: Synthetic oligonucleotide"
  source 1..31: mol_type = other DNA
ttgctgttctaccaagtaatccatatttcta

SEQ ID NO: 121 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 122 - DNA, 31 bp, synthetic construct
  misc_feature 1..31: note = source = /note="Description of Artificial Sequence: Synthetic oligonucleotide"
  source 1..31: mol_type = other DNA
tttctagaggatccccgggtaccgagctcga

SEQ ID NO: 123 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 124 - DNA, 31 bp, synthetic construct
  misc_feature 1..31: note = source = /note="Description of Artificial Sequence: Synthetic oligonucleotide"
  source 1..31: mol_type = other DNA
cgccaatataacaatacaggtattatagttg

SEQ ID NO: 125 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 126 - DNA, 31 bp, synthetic construct; identical to SEQ ID NO: 51
  misc_feature 1..31: note = source = /note="Description of Artificial Sequence: Synthetic oligonucleotide"
  source 1..31: mol_type = other DNA

SEQ ID NO: 127 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 128 - DNA, 31 bp, synthetic construct
  misc_feature 1..31: note = source = /note="Description of Artificial Sequence: Synthetic oligonucleotide"
  source 1..31: mol_type = other DNA
cttctttgttgtcttcgacatgggtaatcct

SEQ ID NO: 129 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 130 - DNA, 31 bp, synthetic construct; identical to SEQ ID NO: 52
  misc_feature 1..31: note = source = /note="Description of Artificial Sequence: Synthetic oligonucleotide"
  source 1..31: mol_type = other DNA

SEQ ID NO: 131 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 132 - DNA, 31 bp, synthetic construct
  misc_feature 1..31: note = source = /note="Description of Artificial Sequence: Synthetic oligonucleotide"
  source 1..31: mol_type = other DNA
tctttgttgtcttcgacatgggtaatcctca

SEQ ID NO: 133 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 134 - DNA, 31 bp, synthetic construct
  misc_feature 1..31: note = source = /note="Description of Artificial Sequence: Synthetic oligonucleotide"
  source 1..31: mol_type = other DNA
taatcctcatgtttgaatggccggcgtctat

SEQ ID NO: 135 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 136 - DNA, 31 bp, synthetic construct
  misc_feature 1..31: note = source = /note="Description of Artificial Sequence: Synthetic oligonucleotide"
  source 1..31: mol_type = other DNA
ttcgacatgggtaatcctcatgtttgaatgg

SEQ ID NO: 137 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 138 - DNA, 31 bp, synthetic construct
  misc_feature 1..31: note = source = /note="Description of Artificial Sequence: Synthetic oligonucleotide"
  source 1..31: mol_type = other DNA
cgaggaagatcaatacataaagagttgaact

SEQ ID NO: 139 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 140 - DNA, 31 bp, synthetic construct
  misc_feature 1..31: note = source = /note="Description of Artificial Sequence: Synthetic oligonucleotide"
  source 1..31: mol_type = other DNA
tggtaaatttcgagagaaagatcgcgaggaa

SEQ ID NO: 141 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 142 - DNA, 31 bp, synthetic construct
  misc_feature 1..31: note = source = /note="Description of Artificial Sequence: Synthetic oligonucleotide"
  source 1..31: mol_type = other DNA
ttgaatggccggcgtctattagtagatgccg

SEQ ID NO: 143 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 144 - DNA, 31 bp, synthetic construct
  misc_feature 1..31: note = source = /note="Description of Artificial Sequence: Synthetic oligonucleotide"
  source 1..31: mol_type = other DNA
aatccatatttctagaggatccccgggtacc

SEQ ID NO: 145 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 146 - DNA, 31 bp, synthetic construct
  misc_feature 1..31: note = source = /note="Description of Artificial Sequence: Synthetic oligonucleotide"
  source 1..31: mol_type = other DNA
tgttctaccaagtaatccatatttctagagg

SEQ ID NO: 147 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 148 - DNA, 31 bp, synthetic construct
  misc_feature 1..31: note = source = /note="Description of Artificial Sequence: Synthetic oligonucleotide"
  source 1..31: mol_type = other DNA
tcgagtagattgctgttctaccaagtaatcc

SEQ ID NO: 149 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 150 - DNA, 31 bp, synthetic construct
  misc_feature 1..31: note = source = /note="Description of Artificial Sequence: Synthetic oligonucleotide"
  source 1..31: mol_type = other DNA
gcatgcctgcaggtcgagtagattgctgttc

SEQ ID NO: 151 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 152 - DNA, 31 bp, synthetic construct
  misc_feature 1..31: note = source = /note="Description of Artificial Sequence: Synthetic oligonucleotide"
  source 1..31: mol_type = other DNA
ttacgccaagcttgcatgcctgcaggtcgag

SEQ ID NO: 153 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 154 - DNA, 31 bp, synthetic construct
  misc_feature 1..31: note = source = /note="Description of Artificial Sequence: Synthetic oligonucleotide"
  source 1..31: mol_type = other DNA
gctatgaccatgattacgccaagcttgcatg

SEQ ID NO: 155 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 156 - DNA, 31 bp, synthetic construct
  misc_feature 1..31: note = source = /note="Description of Artificial Sequence: Synthetic oligonucleotide"
  source 1..31: mol_type = other DNA
aatcctcatgtttgaatggccggcgtctatt

SEQ ID NO: 157 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 158 - DNA, 31 bp, synthetic construct
  misc_feature 1..31: note = source = /note="Description of Artificial Sequence: Synthetic oligonucleotide"
  source 1..31: mol_type = other DNA
cttcgacatgggtaatcctcatgtttgaatg

SEQ ID NO: 159 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 160 - DNA, 31 bp, synthetic construct
  misc_feature 1..31: note = source = /note="Description of Artificial Sequence: Synthetic oligonucleotide"
  source 1..31: mol_type = other DNA
acttctttgttgtcttcgacatgggtaatcc

SEQ ID NO: 161 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 162 - DNA, 31 bp, synthetic construct
  misc_feature 1..31: note = source = /note="Description of Artificial Sequence: Synthetic oligonucleotide"
  source 1..31: mol_type = other DNA
cataaagagttgaacttctttgttgtcttcg

SEQ ID NO: 163 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 164 - DNA, 31 bp, synthetic construct
  misc_feature 1..31: note = source = /note="Description of Artificial Sequence: Synthetic oligonucleotide"
  source 1..31: mol_type = other DNA
aggaagatcaatacataaagagttgaacttc

SEQ ID NO: 165 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 166 - DNA, 31 bp, synthetic construct
  misc_feature 1..31: note = source = /note="Description of Artificial Sequence: Synthetic oligonucleotide"
  source 1..31: mol_type = other DNA
gagaaagatcgcgaggaagatcaatacataa

SEQ ID NO: 167 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 168 - DNA, 31 bp, synthetic construct
  misc_feature 1..31: note = source = /note="Description of Artificial Sequence: Synthetic oligonucleotide"
  source 1..31: mol_type = other DNA
agggtgccggtgatgcggcactcgatcttca

SEQ ID NO: 169 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 170 - DNA, 31 bp, synthetic construct
  misc_feature 1..31: note = source = /note="Description of Artificial Sequence: Synthetic oligonucleotide"
  source 1..31: mol_type = other DNA
ccagctcgaactccacgccgttcagggtgcc

SEQ ID NO: 171 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 172 - DNA, 31 bp, synthetic construct
  misc_feature 1..31: note = source = /note="Description of Artificial Sequence: Synthetic oligonucleotide"
  source 1..31: mol_type = other DNA
ccctctccgccgcccaccagctcgaactcca

SEQ ID NO: 173 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 174 - DNA, 31 bp, synthetic construct
  misc_feature 1..31: note = source = /note="Description of Artificial Sequence: Synthetic oligonucleotide"
  source 1..31: mol_type = other DNA
cggccctgctcgggggtgccctctccgccgc

SEQ ID NO: 175 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 176 - DNA, 31 bp, synthetic construct
  misc_feature 1..31: note = source = /note="Description of Artificial Sequence: Synthetic oligonucleotide"
  source 1..31: mol_type = other DNA
acgtggctcagcaggtaggggctgaaggtca

SEQ ID NO: 177 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 178 - DNA, 31 bp, synthetic construct
  misc_feature 1..31: note = source = /note="Description of Artificial Sequence: Synthetic oligonucleotide"
  source 1..31: mol_type = other DNA
aggtgccgaagtggtagaagccgtagcccat

SEQ ID NO: 179 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 180 - DNA, 31 bp, synthetic construct
  misc_feature 1..31: note = source = /note="Description of Artificial Sequence: Synthetic oligonucleotide"
  source 1..31: mol_type = other DNA
tggcgtgcaggaaggggttctcgtagccgct

SEQ ID NO: 181 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 182 - DNA, 31 bp, synthetic construct
  misc_feature 1..31: note = source = /note="Description of Artificial Sequence: Synthetic oligonucleotide"
  source 1..31: mol_type = other DNA
gtacttctcgatgcgggtgttggtgtagccg

SEQ ID NO: 183 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 184 - DNA, 31 bp, synthetic construct
  misc_feature 1..31: note = source = /note="Description of Artificial Sequence: Synthetic oligonucleotide"
  source 1..31: mol_type = other DNA
gcggccggcctcgtagcggtagctgaagctc

SEQ ID NO: 185 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 186 - DNA, 31 bp, synthetic construct
  misc_feature 1..31: note = source = /note="Description of Artificial Sequence: Synthetic oligonucleotide"
  source 1..31: mol_type = other DNA
aagccggtgcccaccaccttgaagtcgccga

SEQ ID NO: 187 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 188 - DNA, 31 bp, synthetic construct
  misc_feature 1..31: note = source = /note="Description of Artificial Sequence: Synthetic oligonucleotide"
  source 1..31: mol_type = other DNA
aagatcacgctgtcctcggggaagccggtgc

SEQ ID NO: 189 - DNA, 36 bp, synthetic construct
  misc_feature 1..36: note = source = /note="Description of Artificial Sequence: Synthetic oligonucleotide"
  source 1..36: mol_type = other DNA
gtttggaagccgggtcgaagtcaggcccgttaagac

SEQ ID NO: 190 - DNA, 31 bp, synthetic construct; identical to SEQ ID NO: 51
  misc_feature 1..31: note = source = /note="Description of Artificial Sequence: Synthetic oligonucleotide"
  source 1..31: mol_type = other DNA

SEQ ID NO: 191 - DNA, 36 bp, synthetic construct
  misc_feature 1..36: note = source = /note="Description of Artificial Sequence: Synthetic oligonucleotide"
  source 1..36: mol_type = other DNA
cttgaagactaaaggaaggaattaatgtcacggtac

SEQ ID NO: 192 - DNA, 31 bp, synthetic construct; identical to SEQ ID NO: 51
  misc_feature 1..31: note = source = /note="Description of Artificial Sequence: Synthetic oligonucleotide"
  source 1..31: mol_type = other DNA

SEQ ID NO: 193 - DNA, 36 bp, synthetic construct; identical to SEQ ID NO: 189
  misc_feature 1..36: note = source = /note="Description of Artificial Sequence: Synthetic oligonucleotide"
  source 1..36: mol_type = other DNA

SEQ ID NO: 194 - DNA, 31 bp, synthetic construct; identical to SEQ ID NO: 124
  misc_feature 1..31: note = source = /note="Description of Artificial Sequence: Synthetic oligonucleotide"
  source 1..31: mol_type = other DNA

SEQ ID NO: 195 - DNA, 36 bp, synthetic construct; identical to SEQ ID NO: 191
  misc_feature 1..36: note = source = /note="Description of Artificial Sequence: Synthetic oligonucleotide"
  source 1..36: mol_type = other DNA

SEQ ID NO: 196 - DNA, 31 bp, synthetic construct; identical to SEQ ID NO: 124
  misc_feature 1..31: note = source = /note="Description of Artificial Sequence: Synthetic oligonucleotide"
  source 1..31: mol_type = other DNA

SEQ ID NO: 197 - DNA, 34 bp, synthetic construct
  misc_feature 1..34: note = source = /note="Description of Artificial Sequence: Synthetic oligonucleotide"
  source 1..34: mol_type = other DNA
gttggaaagccggttttctttgatgtcacggaac

SEQ ID NO: 198 - DNA, 31 bp, synthetic construct; identical to SEQ ID NO: 51
  misc_feature 1..31: note = source = /note="Description of Artificial Sequence: Synthetic oligonucleotide"
  source 1..31: mol_type = other DNA

SEQ ID NO: 199 - DNA, 31 bp, synthetic construct
  misc_feature 1..31: note = source = /note="Description of Artificial Sequence: Synthetic oligonucleotide"
  source 1..31: mol_type = other DNA
ggaaagccggttttctttgatgtcacggaac

SEQ ID NO: 200 - DNA, 31 bp, synthetic construct; identical to SEQ ID NO: 51
  misc_feature 1..31: note = source = /note="Description of Artificial Sequence: Synthetic oligonucleotide"
  source 1..31: mol_type = other DNA

SEQ ID NO: 201 - DNA, 28 bp, synthetic construct
  misc_feature 1..28: note = source = /note="Description of Artificial Sequence: Synthetic oligonucleotide"
  source 1..28: mol_type = other DNA
aagccggttttctttgatgtcacggaac

SEQ ID NO: 202 - DNA, 31 bp, synthetic construct; identical to SEQ ID NO: 51
  misc_feature 1..31: note = source = /note="Description of Artificial Sequence: Synthetic oligonucleotide"
  source 1..31: mol_type = other DNA

SEQ ID NO: 203 - DNA, 25 bp, synthetic construct
  misc_feature 1..25: note = source = /note="Description of Artificial Sequence: Synthetic oligonucleotide"
  source 1..25: mol_type = other DNA
ccggttttctttgatgtcacggaac

SEQ ID NO: 204 - DNA, 31 bp, synthetic construct; identical to SEQ ID NO: 51
  misc_feature 1..31: note = source = /note="Description of Artificial Sequence: Synthetic oligonucleotide"
  source 1..31: mol_type = other DNA

SEQ ID NO: 205 - DNA, 22 bp, synthetic construct
  misc_feature 1..22: note = source = /note="Description of Artificial Sequence: Synthetic oligonucleotide"
  source 1..22: mol_type = other DNA
gttttctttgatgtcacggaac

SEQ ID NO: 206 - DNA, 31 bp, synthetic construct; identical to SEQ ID NO: 51
  misc_feature 1..31: note = source = /note="Description of Artificial Sequence: Synthetic oligonucleotide"
  source 1..31: mol_type = other DNA

SEQ ID NO: 207 - DNA, 18 bp, synthetic construct
  misc_feature 1..18: note = source = /note="Description of Artificial Sequence: Synthetic oligonucleotide"
  source 1..18: mol_type = other DNA
tctttgatgtcacggaac

SEQ ID NO: 208 - DNA, 31 bp, synthetic construct; identical to SEQ ID NO: 51
  misc_feature 1..31: note = source = /note="Description of Artificial Sequence: Synthetic oligonucleotide"
  source 1..31: mol_type = other DNA

SEQ ID NO: 209 - DNA, 15 bp, synthetic construct
  misc_feature 1..15: note = source = /note="Description of Artificial Sequence: Synthetic oligonucleotide"
  source 1..15: mol_type = other DNA
ttgatgtcacggaac

SEQ ID NO: 210 - DNA, 31 bp, synthetic construct; identical to SEQ ID NO: 51
  misc_feature 1..31: note = source = /note="Description of Artificial Sequence: Synthetic oligonucleotide"
  source 1..31: mol_type = other DNA

SEQ ID NO: 211 - DNA, 12 bp, synthetic construct
  misc_feature 1..12: note = source = /note="Description of Artificial Sequence: Synthetic oligonucleotide"
  source 1..12: mol_type = other DNA
atgtcacggaac

SEQ ID NO: 212 - DNA, 31 bp, synthetic construct; identical to SEQ ID NO: 51
  misc_feature 1..31: note = source = /note="Description of Artificial Sequence: Synthetic oligonucleotide"
  source 1..31: mol_type = other DNA

SEQ ID NO: 213 - skipped

SEQ ID NO: 214 - DNA, 31 bp, synthetic construct; identical to SEQ ID NO: 51
  misc_feature 1..31: note = source = /note="Description of Artificial Sequence: Synthetic oligonucleotide"
  source 1..31: mol_type = other DNA

SEQ ID NO: 215 - skipped

SEQ ID NO: 216 - DNA, 31 bp, synthetic construct; identical to SEQ ID NO: 51
  misc_feature 1..31: note = source = /note="Description of Artificial Sequence: Synthetic oligonucleotide"
  source 1..31: mol_type = other DNA

SEQ ID NO: 217 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 218 - DNA, 30 bp, synthetic construct
  misc_feature 1..30: note = source = /note="Description of Artificial Sequence: Synthetic oligonucleotide"
  source 1..30: mol_type = other DNA
ctttgttgtcttcgacatgggtaatcctca

SEQ ID NO: 219 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 220 - DNA, 28 bp, synthetic construct
  misc_feature 1..28: note = source = /note="Description of Artificial Sequence: Synthetic oligonucleotide"
  source 1..28: mol_type = other DNA
ctttgttgtcttcgacatgggtaatcct

SEQ ID NO: 221 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 222 - DNA, 25 bp, synthetic construct
  misc_feature 1..25: note = source = /note="Description of Artificial Sequence: Synthetic oligonucleotide"
  source 1..25: mol_type = other DNA
ctttgttgtcttcgacatgggtaat

SEQ ID NO: 223 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 224 - DNA, 22 bp, synthetic construct
  misc_feature 1..22: note = source = /note="Description of Artificial Sequence: Synthetic oligonucleotide"
  source 1..22: mol_type = other DNA
ctttgttgtcttcgacatgggt

SEQ ID NO: 225 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 226 - DNA, 19 bp, synthetic construct
  misc_feature 1..19: note = source = /note="Description of Artificial Sequence: Synthetic oligonucleotide"
  source 1..19: mol_type = other DNA
ctttgttgtcttcgacatg

SEQ ID NO: 227 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 228 - DNA, 16 bp, synthetic construct
  misc_feature 1..16: note = source = /note="Description of Artificial Sequence: Synthetic oligonucleotide"
  source 1..16: mol_type = other DNA
ctttgttgtcttcgac

SEQ ID NO: 229 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 230 - DNA, 13 bp, synthetic construct
  misc_feature 1..13: note = source = /note="Description of Artificial Sequence: Synthetic oligonucleotide"
  source 1..13: mol_type = other DNA
ctttgttgtcttc

SEQ ID NO: 231 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 232 - DNA, 10 bp, synthetic construct
  misc_feature 1..10: note = source = /note="Description of Artificial Sequence: Synthetic oligonucleotide"
  source 1..10: mol_type = other DNA
ctttgttgtc

SEQ ID NO: 233 - DNA, 15 bp, synthetic construct
  misc_feature 1..15: note = source = /note="Description of Artificial Sequence: Synthetic oligonucleotide"
  source 1..15: mol_type = other DNA
ttgatgtcacggaac

SEQ ID NO: 234 - DNA, 31 bp, synthetic construct; identical to SEQ ID NO: 51
  misc_feature 1..31: note = source = /note="Description of Artificial Sequence: Synthetic oligonucleotide"
  source 1..31: mol_type = other DNA

SEQ ID NO: 235 - DNA, 15 bp, synthetic construct
  misc_feature 1..15: note = source = /note="Description of Artificial Sequence: Synthetic oligonucleotide"
  source 1..15: mol_type = other DNA
atgatgtcacggaac

SEQ ID NO: 236 - DNA, 31 bp, synthetic construct; identical to SEQ ID NO: 51
  misc_feature 1..31: note = source = /note="Description of Artificial Sequence: Synthetic oligonucleotide"
  source 1..31: mol_type = other DNA

SEQ ID NO: 237 - DNA, 15 bp, synthetic construct
  misc_feature 1..15: note = source = /note="Description of Artificial Sequence: Synthetic oligonucleotide"
  source 1..15: mol_type = other DNA
tagatgtcacggaac

SEQ ID NO: 238 - DNA, 31 bp, synthetic construct; identical to SEQ ID NO: 51
  misc_feature 1..31: note = source = /note="Description of Artificial Sequence: Synthetic oligonucleotide"
  source 1..31: mol_type = other DNA

SEQ ID NO: 239 - DNA, 15 bp, synthetic construct
  misc_feature 1..15: note = source = /note="Description of Artificial Sequence: Synthetic oligonucleotide"
  source 1..15: mol_type = other DNA
ttcatgtcacggaac

SEQ ID NO: 240 - DNA, 31 bp, synthetic construct; identical to SEQ ID NO: 51
  misc_feature 1..31: note = source = /note="Description of Artificial Sequence: Synthetic oligonucleotide"
  source 1..31: mol_type = other DNA

SEQ ID NO: 241 - DNA, 15 bp, synthetic construct
  misc_feature 1..15: note = source = /note="Description of Artificial Sequence: Synthetic oligonucleotide"
  source 1..15: mol_type = other DNA
ttgttgtcacggaac

SEQ ID NO: 242 - DNA, 31 bp, synthetic construct; identical to SEQ ID NO: 51
  misc_feature 1..31: note = source = /note="Description of Artificial Sequence: Synthetic oligonucleotide"
  source 1..31: mol_type = other DNA

SEQ ID NO: 243 - DNA, 15 bp, synthetic construct
  misc_feature 1..15: note = source = /note="Description of Artificial Sequence: Synthetic oligonucleotide"
  source 1..15: mol_type = other DNA
ttgaagtcacggaac

SEQ ID NO: 244 - DNA, 31 bp, synthetic construct; identical to SEQ ID NO: 51
  misc_feature 1..31: note = source = /note="Description of Artificial Sequence: Synthetic oligonucleotide"
  source 1..31: mol_type = other DNA

SEQ ID NO: 245 - DNA, 15 bp, synthetic construct
  misc_feature 1..15: note = source = /note="Description of Artificial Sequence: Synthetic oligonucleotide"
  source 1..15: mol_type = other DNA
ttgatctcacggaac

SEQ ID NO: 246 - DNA, 31 bp, synthetic construct; identical to SEQ ID NO: 51
  misc_feature 1..31: note = source = /note="Description of Artificial Sequence: Synthetic oligonucleotide"
  source 1..31: mol_type = other DNA

SEQ ID NO: 247 - DNA, 15 bp, synthetic construct
  misc_feature 1..15: note = source = /note="Description of Artificial Sequence: Synthetic oligonucleotide"
  source 1..15: mol_type = other DNA
ttgatgacacggaac

SEQ ID NO: 248 - DNA, 31 bp, synthetic construct; identical to SEQ ID NO: 51
  misc_feature 1..31: note = source = /note="Description of Artificial Sequence: Synthetic oligonucleotide"
  source 1..31: mol_type = other DNA

SEQ ID NO: 249 - DNA, 15 bp, synthetic construct
  misc_feature 1..15: note = source = /note="Description of Artificial Sequence: Synthetic oligonucleotide"
  source 1..15: mol_type = other DNA
ttgatgtgacggaac

SEQ ID NO: 250 - DNA, 31 bp, synthetic construct; identical to SEQ ID NO: 51
  misc_feature 1..31: note = source = /note="Description of Artificial Sequence: Synthetic oligonucleotide"
  source 1..31: mol_type = other DNA

SEQ ID NO: 251 - DNA, 15 bp, synthetic construct
  misc_feature 1..15: note = source = /note="Description of Artificial Sequence: Synthetic oligonucleotide"
  source 1..15: mol_type = other DNA
ttgatgtctcggaac

SEQ ID NO: 252 - DNA, 31 bp, synthetic construct; identical to SEQ ID NO: 51
  misc_feature 1..31: note = source = /note="Description of Artificial Sequence: Synthetic oligonucleotide"
  source 1..31: mol_type = other DNA

SEQ ID NO: 253 - DNA, 15 bp, synthetic construct
  misc_feature 1..15: note = source = /note="Description of Artificial Sequence: Synthetic oligonucleotide"
  source 1..15: mol_type = other DNA
ttgatgtcagggaac

SEQ ID NO: 254 - DNA, 31 bp, synthetic construct; identical to SEQ ID NO: 51
  misc_feature 1..31: note = source = /note="Description of Artificial Sequence: Synthetic oligonucleotide"
  source 1..31: mol_type = other DNA

SEQ ID NO: 255 - DNA, 15 bp, synthetic construct
  misc_feature 1..15: note = source = /note="Description of Artificial Sequence: Synthetic oligonucleotide"
  source 1..15: mol_type = other DNA
ttgatgtcaccgaac

SEQ ID NO: 256 - DNA, 31 bp, synthetic construct; identical to SEQ ID NO: 51
  misc_feature 1..31: note = source = /note="Description of Artificial Sequence: Synthetic oligonucleotide"
  source 1..31: mol_type = other DNA

SEQ ID NO: 257 - DNA, 15 bp, synthetic construct
  misc_feature 1..15: note = source = /note="Description of Artificial Sequence: Synthetic oligonucleotide"
  source 1..15: mol_type = other DNA
ttgatgtcacgcaac

SEQ ID NO: 258 - DNA, 31 bp, synthetic construct; identical to SEQ ID NO: 51
  misc_feature 1..31: note = source = /note="Description of Artificial Sequence: Synthetic oligonucleotide"
  source 1..31: mol_type = other DNA

SEQ ID NO: 259 - DNA, 15 bp, synthetic construct
  misc_feature 1..15: note = source = /note="Description of Artificial Sequence: Synthetic oligonucleotide"
  source 1..15: mol_type = other DNA
ttgatgtcacggtac

SEQ ID NO: 260 - DNA, 31 bp, synthetic construct; identical to SEQ ID NO: 51
  misc_feature 1..31: note = source = /note="Description of Artificial Sequence: Synthetic oligonucleotide"
  source 1..31: mol_type = other DNA

SEQ ID NO: 261 - DNA, 15 bp, synthetic construct
  misc_feature 1..15: note = source = /note="Description of Artificial Sequence: Synthetic oligonucleotide"
  source 1..15: mol_type = other DNA
ttgatgtcacggatc

SEQ ID NO: 262 - DNA, 31 bp, synthetic construct; identical to SEQ ID NO: 51
  misc_feature 1..31: note = source = /note="Description of Artificial Sequence: Synthetic oligonucleotide"
  source 1..31: mol_type = other DNA

SEQ ID NO: 263 - DNA, 15 bp, synthetic construct
  misc_feature 1..15: note = source = /note="Description of Artificial Sequence: Synthetic oligonucleotide"
  source 1..15: mol_type = other DNA
ttgatgtcacggaag

SEQ ID NO: 264 - DNA, 31 bp, synthetic construct; identical to SEQ ID NO: 51
  misc_feature 1..31: note = source = /note="Description of Artificial Sequence: Synthetic oligonucleotide"
  source 1..31: mol_type = other DNA

SEQ ID NO: 265 - DNA, 15 bp, synthetic construct
  misc_feature 1..15: note = source = /note="Description of Artificial Sequence: Synthetic oligonucleotide"
  source 1..15: mol_type = other DNA
ttgatgtcacggaac

SEQ ID NO: 266 - DNA, 31 bp, synthetic construct; identical to SEQ ID NO: 51
  misc_feature 1..31: note = source = /note="Description of Artificial Sequence: Synthetic oligonucleotide"
  source 1..31: mol_type = other DNA

SEQ ID NO: 267 - DNA, 15 bp, synthetic construct
  misc_feature 1..15: note = source = /note="Description of Artificial Sequence: Synthetic oligonucleotide"
  source 1..15: mol_type = other DNA
aagatgtcacggaac

SEQ ID NO: 268 - DNA, 31 bp, synthetic construct; identical to SEQ ID NO: 51
  misc_feature 1..31: note = source = /note="Description of Artificial Sequence: Synthetic oligonucleotide"
  source 1..31: mol_type = other DNA

SEQ ID NO: 269 - DNA, 15 bp, synthetic construct
  misc_feature 1..15: note = source = /note="Description of Artificial Sequence: Synthetic oligonucleotide"
  source 1..15: mol_type = other DNA
tacatgtcacggaac

SEQ ID NO: 270 - DNA, 31 bp, synthetic construct; identical to SEQ ID NO: 51
  misc_feature 1..31: note = source = /note="Description of Artificial Sequence: Synthetic oligonucleotide"
  source 1..31: mol_type = other DNA

SEQ ID NO: 271 - DNA, 15 bp, synthetic construct
  misc_feature 1..15: note = source = /note="Description of Artificial Sequence: Synthetic oligonucleotide"
  source 1..15: mol_type = other DNA
ttcttgtcacggaac

SEQ ID NO: 272 - DNA, 31 bp, synthetic construct; identical to SEQ ID NO: 51
  misc_feature 1..31: note = source = /note="Description of Artificial Sequence: Synthetic oligonucleotide"
  source 1..31: mol_type = other DNA

SEQ ID NO: 273 - DNA, 15 bp, synthetic construct
  misc_feature 1..15: note = source = /note="Description of Artificial Sequence: Synthetic oligonucleotide"
  source 1..15: mol_type = other DNA
ttgtagtcacggaac

SEQ ID NO: 274 - DNA, 31 bp, synthetic construct; identical to SEQ ID NO: 51
  misc_feature 1..31: note = source = /note="Description of Artificial Sequence: Synthetic oligonucleotide"
  source 1..31: mol_type = other DNA

SEQ ID NO: 275 - DNA, 15 bp, synthetic construct
  misc_feature 1..15: note = source = /note="Description of Artificial Sequence: Synthetic oligonucleotide"
  source 1..15: mol_type = other DNA
ttgaactcacggaac

SEQ ID NO: 276 - DNA, 31 bp, synthetic construct; identical to SEQ ID NO: 51
  misc_feature 1..31: note = source = /note="Description of Artificial Sequence: Synthetic oligonucleotide"
  source 1..31: mol_type = other DNA

SEQ ID NO: 277 - DNA, 15 bp, synthetic construct
  misc_feature 1..15: note = source = /note="Description of Artificial Sequence: Synthetic oligonucleotide"
  source 1..15: mol_type = other DNA
ttgatcacacggaac

SEQ ID NO: 278 - DNA, 31 bp, synthetic construct; identical to SEQ ID NO: 51
  misc_feature 1..31: note = source = /note="Description of Artificial Sequence: Synthetic oligonucleotide"
  source 1..31: mol_type = other DNA

SEQ ID NO: 279 - DNA, 15 bp, synthetic construct
  misc_feature 1..15: note = source = /note="Description of Artificial Sequence: Synthetic oligonucleotide"
  source 1..15: mol_type = other DNA
ttgatgagacggaac

SEQ ID NO: 280 - DNA, 31 bp, synthetic construct; identical to SEQ ID NO: 51
  misc_feature 1..31: note = source = /note="Description of Artificial Sequence: Synthetic oligonucleotide"
  source 1..31: mol_type = other DNA

SEQ ID NO: 281 - DNA, 15 bp, synthetic construct
  misc_feature 1..15: note = source = /note="Description of Artificial Sequence: Synthetic oligonucleotide"
  source 1..15: mol_type = other DNA
ttgatgtgtcggaac

SEQ ID NO: 282 - DNA, 31 bp, synthetic construct; identical to SEQ ID NO: 51
  misc_feature 1..31: note = source = /note="Description of Artificial Sequence: Synthetic oligonucleotide"
  source 1..31: mol_type = other DNA

SEQ ID NO: 283 - DNA, 15 bp, synthetic construct
  misc_feature 1..15: note = source = /note="Description of Artificial Sequence: Synthetic oligonucleotide"
  source 1..15: mol_type = other DNA
ttgatgtctgggaac

SEQ ID NO: 284 - DNA, 31 bp, synthetic construct; identical to SEQ ID NO: 51
  misc_feature 1..31: note = source = /note="Description of Artificial Sequence: Synthetic oligonucleotide"
  source 1..31: mol_type = other DNA

SEQ ID NO: 285 - DNA, 15 bp, synthetic construct
  misc_feature 1..15: note = source = /note="Description of Artificial Sequence: Synthetic oligonucleotide"
  source 1..15: mol_type = other DNA
ttgatgtcagcgaac

SEQ ID NO: 286 - DNA, 31 bp, synthetic construct; identical to SEQ ID NO: 51
  misc_feature 1..31: note = source = /note="Description of Artificial Sequence: Synthetic oligonucleotide"
  source 1..31: mol_type = other DNA

SEQ ID NO: 287 - DNA, 15 bp, synthetic construct
  misc_feature 1..15: note = source = /note="Description of Artificial Sequence: Synthetic oligonucleotide"
  source 1..15: mol_type = other DNA
ttgatgtcacccaac

SEQ ID NO: 288 - DNA, 31 bp, synthetic construct; identical to SEQ ID NO: 51
  misc_feature 1..31: note = source = /note="Description of Artificial Sequence: Synthetic oligonucleotide"
  source 1..31: mol_type = other DNA

SEQ ID NO: 289 - DNA, 15 bp, synthetic construct
  misc_feature 1..15: note = source = /note="Description of Artificial Sequence: Synthetic oligonucleotide"
  source 1..15: mol_type = other DNA
ttgatgtcacgctac

SEQ ID NO: 290 - DNA, 31 bp, synthetic construct; identical to SEQ ID NO: 51
  misc_feature 1..31: note = source = /note="Description of Artificial Sequence: Synthetic oligonucleotide"
  source 1..31: mol_type = other DNA

SEQ ID NO: 291 - DNA, 15 bp, synthetic construct
  misc_feature 1..15: note = source = /note="Description of Artificial Sequence: Synthetic oligonucleotide"
  source 1..15: mol_type = other DNA
ttgatgtcacggttc

SEQ ID NO: 292 - DNA, 31 bp, synthetic construct; identical to SEQ ID NO: 51
  misc_feature 1..31: note = source = /note="Description of Artificial Sequence: Synthetic oligonucleotide"
  source 1..31: mol_type = other DNA

SEQ ID NO: 293 - DNA, 15 bp, synthetic construct
  misc_feature 1..15: note = source = /note="Description of Artificial Sequence: Synthetic oligonucleotide"
  source 1..15: mol_type = other DNA
ttgatgtcacggatg

SEQ ID NO: 294 - DNA, 31 bp, synthetic construct; identical to SEQ ID NO: 51
  misc_feature 1..31: note = source = /note="Description of Artificial Sequence: Synthetic oligonucleotide"
  source 1..31: mol_type = other DNA

SEQ ID NO: 295 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 296 - DNA, 31 bp, synthetic construct
  misc_feature 1..31: note = source = /note="Description of Artificial Sequence: Synthetic oligonucleotide"
  source 1..31: mol_type = other DNA
accaaacgtaatgcggggtgcatttcgctga

SEQ ID NO: 297 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 298 - DNA, 31 bp, synthetic construct
  misc_feature 1..31: note = source = /note="Description of Artificial Sequence: Synthetic oligonucleotide"
  source 1..31: mol_type = other DNA
gttgaatctgagggtccaccaaacgtaatgc

SEQ ID NO: 299 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 300 - DNA, 31 bp, synthetic construct
  misc_feature 1..31: note = source = /note="Description of Artificial Sequence: Synthetic oligonucleotide"
  source 1..31: mol_type = other DNA
cattctggttactgccagttgaatctgaggg

SEQ ID NO: 301 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 302 - DNA, 31 bp, synthetic construct
  misc_feature 1..31: note = source = /note="Description of Artificial Sequence: Synthetic oligonucleotide"
  source 1..31: mol_type = other DNA
gccccactgcgttctccattctggttactgc

SEQ ID NO: 303 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 304 - DNA, 31 bp, synthetic construct
  misc_feature 1..31: note = source = /note="Description of Artificial Sequence: Synthetic oligonucleotide"
  source 1..31: mol_type = other DNA
acgttgttttgatcgcgccccactgcgttct

SEQ ID NO: 305 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 306 - DNA, 31 bp, synthetic construct
  misc_feature 1..31: note = source = /note="Description of Artificial Sequence: Synthetic oligonucleotide"
  source 1..31: mol_type = other DNA
ggtaaaccttggggccgacgttgttttgatc

SEQ ID NO: 307 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 308 - DNA, 31 bp, synthetic construct
  misc_feature 1..31: note = source = /note="Description of Artificial Sequence: Synthetic oligonucleotide"
  source 1..31: mol_type = other DNA
agacgcagtattattgggtaaaccttggggc

SEQ ID NO: 309 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 310 - DNA, 31 bp, synthetic construct
  misc_feature 1..31: note = source = /note="Description of Artificial Sequence: Synthetic oligonucleotide"
  source 1..31: mol_type = other DNA
tgagagcggtgaaccaagacgcagtattatt

SEQ ID NO: 311 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 312 - DNA, 31 bp, synthetic construct
  misc_feature 1..31: note = source = /note="Description of Artificial Sequence: Synthetic oligonucleotide"
  source 1..31: mol_type = other DNA
ccgggaagtttttgagcgaaaaccagacata

SEQ ID NO: 313 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 314 - DNA, 31 bp, synthetic construct
  misc_feature 1..31: note = source = /note="Description of Artificial Sequence: Synthetic oligonucleotide"
  source 1..31: mol_type = other DNA
cgtgctgttgtcatttccgggaagtttttga

SEQ ID NO: 315 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 316 - DNA, 31 bp, synthetic construct
  misc_feature 1..31: note = source = /note="Description of Artificial Sequence: Synthetic oligonucleotide"
  source 1..31: mol_type = other DNA
caaggcacagcgttgccgtgctgttgtcatt

SEQ ID NO: 317 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 318 - DNA, 31 bp, synthetic construct
  misc_feature 1..31: note = source = /note="Description of Artificial Sequence: Synthetic oligonucleotide"
  source 1..31: mol_type = other DNA
ggtactgcatggtgcccaaggcacagcgttg

SEQ ID NO: 319 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 320 - DNA, 31 bp, synthetic construct
  misc_feature 1..31: note = source = /note="Description of Artificial Sequence: Synthetic oligonucleotide"
  source 1..31: mol_type = other DNA
tcactatcgttccgtttggtactgcatggtg

SEQ ID NO: 321 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 322 - DNA, 31 bp, synthetic construct
  misc_feature 1..31: note = source = /note="Description of Artificial Sequence: Synthetic oligonucleotide"
  source 1..31: mol_type = other DNA
gtcattcgtgattgttttcactatcgttccg

SEQ ID NO: 323 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 324 - DNA, 31 bp, synthetic construct
  misc_feature 1..31: note = source = /note="Description of Artificial Sequence: Synthetic oligonucleotide"
  source 1..31: mol_type = other DNA
agtgacttcaatttggtcattcgtgattgtt

SEQ ID NO: 325 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 326 - DNA, 31 bp, synthetic construct
  misc_feature 1..31: note = source = /note="Description of Artificial Sequence: Synthetic oligonucleotide"
  source 1..31: mol_type = other DNA
accagttcagtagcattagtgacttcaattt

SEQ ID NO: 327 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 328 - DNA, 31 bp, synthetic construct
  misc_feature 1..31: note = source = /note="Description of Artificial Sequence: Synthetic oligonucleotide"
  source 1..31: mol_type = other DNA
tggggtgcaggtgctccacggtggcgttgct

SEQ ID NO: 329 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 330 - DNA, 31 bp, synthetic construct
  misc_feature 1..31: note = source = /note="Description of Artificial Sequence: Synthetic oligonucleotide"
  source 1..31: mol_type = other DNA
aaggtgcgggcgaagctgcccaccagcacgt

SEQ ID NO: 331 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 332 - DNA, 31 bp, synthetic construct
  misc_feature 1..31: note = source = /note="Description of Artificial Sequence: Synthetic oligonucleotide"
  source 1..31: mol_type = other DNA
gtccaccacgaagctgtagtagccgccgtcg

SEQ ID NO: 333 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 334 - DNA, 31 bp, synthetic construct
  misc_feature 1..31: note = source = /note="Description of Artificial Sequence: Synthetic oligonucleotide"
  source 1..31: mol_type = other DNA
tgctggggtggatggcgctcttgaagtgcat

SEQ ID NO: 335 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 336 - DNA, 31 bp, synthetic construct
  misc_feature 1..31: note = source = /note="Description of Artificial Sequence: Synthetic oligonucleotide"
  source 1..31: mol_type = other DNA
ggaaggcgaacatggggcccccgttctgcag

SEQ ID NO: 337 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 338 - DNA, 31 bp, synthetic construct; identical to SEQ ID NO: 156
  misc_feature 1..31: note = source = /note="Description of Artificial Sequence: Synthetic oligonucleotide"
  source 1..31: mol_type = other DNA

SEQ ID NO: 339 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 340 - DNA, 31 bp, synthetic construct
  misc_feature 1..31: note = source = /note="Description of Artificial Sequence: Synthetic oligonucleotide"
  source 1..31: mol_type = other DNA
ggtaatcctcatgtttgaatggccggcgtct

SEQ ID NO: 341 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 342 - DNA, 31 bp, synthetic construct
  misc_feature 1..31: note = source = /note="Description of Artificial Sequence: Synthetic oligonucleotide"
  source 1..31: mol_type = other DNA
atgggtaatcctcatgtttgaatggccggcg

SEQ ID NO: 343 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 344 - DNA, 31 bp, synthetic construct
  misc_feature 1..31: note = source = /note="Description of Artificial Sequence: Synthetic oligonucleotide"
  source 1..31: mol_type = other DNA
gacatgggtaatcctcatgtttgaatggccg

SEQ ID NO: 345 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 346 - DNA, 31 bp, synthetic construct; identical to SEQ ID NO: 136
  misc_feature 1..31: note = source = /note="Description of Artificial Sequence: Synthetic oligonucleotide"
  source 1..31: mol_type = other DNA

SEQ ID NO: 347 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 348 - DNA, 31 bp, synthetic construct
  misc_feature 1..31: note = source = /note="Description of Artificial Sequence: Synthetic oligonucleotide"
  source 1..31: mol_type = other DNA
gtcttcgacatgggtaatcctcatgtttgaa

SEQ ID NO: 349 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 350 - DNA, 31 bp, synthetic construct
  misc_feature 1..31: note = source = /note="Description of Artificial Sequence: Synthetic oligonucleotide"
  source 1..31: mol_type = other DNA
gttgtcttcgacatgggtaatcctcatgttt

SEQ ID NO: 351 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 352 - DNA, 31 bp, synthetic construct
  misc_feature 1..31: note = source = /note="Description of Artificial Sequence: Synthetic oligonucleotide"
  source 1..31: mol_type = other DNA
tttgttgtcttcgacatgggtaatcctcatg

SEQ ID NO: 353 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 354 - DNA, 31 bp, synthetic construct
  misc_feature 1..31: note = source = /note="Description of Artificial Sequence: Synthetic oligonucleotide"
  source 1..31: mol_type = other DNA
ttctttgttgtcttcgacatgggtaatcctc

SEQ ID NO: 355 - DNA, 35 bp, synthetic construct; identical to SEQ ID NO: 111
  misc_feature 1..35: note = source = /note="Description of Artificial Sequence: Synthetic oligonucleotide"
  source 1..35: mol_type = other DNA

SEQ ID NO: 356 - DNA, 31 bp, synthetic construct
  misc_feature 1..31: note = source = /note="Description of Artificial Sequence: Synthetic oligonucleotide"
  source 1..31: mol_type = other DNA
aacttctttgttgtcttcgacatgggtaatc

SEQ ID NO: 357 - DNA, 36 bp, synthetic construct; identical to SEQ ID NO: 191
  misc_feature 1..36: note = source = /note="Description of Artificial Sequence: Synthetic oligonucleotide"
  source 1..36: mol_type = other DNA

SEQ ID NO: 358 - DNA, 31 bp, synthetic construct; identical to SEQ ID NO: 156
  misc_feature 1..31: note = source = /note="Description of Artificial Sequence: Synthetic oligonucleotide"
  source 1..31: mol_type = other DNA

SEQ ID NO: 359 - DNA, 36 bp, synthetic construct; identical to SEQ ID NO: 191
  misc_feature 1..36: note = source = /note="Description of Artificial Sequence: Synthetic oligonucleotide"
  source 1..36: mol_type = other DNA

SEQ ID NO: 360 - DNA, 31 bp, synthetic construct; identical to SEQ ID NO: 340
  misc_feature 1..31: note = source = /note="Description of Artificial Sequence: Synthetic oligonucleotide"
  source 1..31: mol_type = other DNA

SEQ ID NO: 361 - DNA, 36 bp, synthetic construct; identical to SEQ ID NO: 191
  misc_feature 1..36: note = source = /note="Description of Artificial Sequence: Synthetic oligonucleotide"
  source 1..36: mol_type = other DNA

SEQ ID NO: 362 - DNA, 31 bp, synthetic construct; identical to SEQ ID NO: 342
  misc_feature 1..31: note = source = /note="Description of Artificial Sequence: Synthetic oligonucleotide"
  source 1..31: mol_type = other DNA

SEQ ID NO: 363 - DNA, 36 bp, synthetic construct; identical to SEQ ID NO: 191
  misc_feature 1..36: note = source = /note="Description of Artificial Sequence: Synthetic oligonucleotide"
  source 1..36: mol_type = other DNA

SEQ ID NO: 364 - DNA, 31 bp, synthetic construct; identical to SEQ ID NO: 344
  misc_feature 1..31: note = source = /note="Description of Artificial Sequence: Synthetic oligonucleotide"
  source 1..31: mol_type = other DNA

SEQ ID NO: 365 - DNA, 36 bp, synthetic construct; identical to SEQ ID NO: 191
  misc_feature 1..36: note = source = /note="Description of Artificial Sequence: Synthetic oligonucleotide"
  source 1..36: mol_type = other DNA

SEQ ID NO: 366 - DNA, 31 bp, synthetic construct; identical to SEQ ID NO: 136
  misc_feature 1..31: note = source = /note="Description of Artificial Sequence: Synthetic oligonucleotide"
  source 1..31: mol_type = other DNA

SEQ ID NO: 367 - DNA, 36 bp, synthetic construct; identical to SEQ ID NO: 191
  misc_feature 1..36: note = source = /note="Description of Artificial Sequence: Synthetic oligonucleotide"
  source 1..36: mol_type = other DNA

SEQ ID NO: 368 - DNA, 31 bp, synthetic construct; identical to SEQ ID NO: 348
  misc_feature 1..31: note = source = /note="Description of Artificial Sequence: Synthetic oligonucleotide"
  source 1..31: mol_type = other DNA

SEQ ID NO: 369 - DNA, 36 bp, synthetic construct; identical to SEQ ID NO: 191
  misc_feature 1..36: note = source = /note="Description of Artificial Sequence: Synthetic oligonucleotide"
  source 1..36: mol_type = other DNA

SEQ ID NO: 370 - DNA, 31 bp, synthetic construct; identical to SEQ ID NO: 350
  misc_feature 1..31: note = source = /note="Description of Artificial Sequence: Synthetic oligonucleotide"
  source 1..31: mol_type = other DNA

SEQ ID NO: 371 - DNA, 36 bp, synthetic construct; identical to SEQ ID NO: 191
  misc_feature 1..36: note = source = /note="Description of Artificial Sequence: Synthetic oligonucleotide"
  source 1..36: mol_type = other DNA

SEQ ID NO: 372 - DNA, 31 bp, synthetic construct; identical to SEQ ID NO: 352
  misc_feature 1..31: note = source = /note="Description of Artificial Sequence: Synthetic oligonucleotide"
  source 1..31: mol_type = other DNA

SEQ ID NO: 373 - DNA, 36 bp, synthetic construct; identical to SEQ ID NO: 191
  misc_feature 1..36: note = source = /note="Description of Artificial Sequence: Synthetic oligonucleotide"
  source 1..36: mol_type = other DNA

SEQ ID NO: 374 - DNA, 31 bp, synthetic construct; identical to SEQ ID NO: 354
  misc_feature 1..31: note = source = /note="Description of Artificial Sequence: Synthetic oligonucleotide"
  source 1..31: mol_type = other DNA

SEQ ID NO: 375 - DNA, 36 bp, synthetic construct; identical to SEQ ID NO: 191
  misc_feature 1..36: note = source = /note="Description of Artificial Sequence: Synthetic oligonucleotide"
  source 1..36: mol_type = other DNA

SEQ ID NO: 376 - DNA, 31 bp, synthetic construct; identical to SEQ ID NO: 356
  misc_feature 1..31: note = source = /note="Description of Artificial Sequence: Synthetic oligonucleotide"
  source 1..31: mol_type = other DNA

SEQ ID NO: 377 - DNA, 36 bp, synthetic construct; identical to SEQ ID NO: 191
  misc_feature 1..36: note = source = /note="Description of Artificial Sequence: Synthetic oligonucleotide"
  source 1..36: mol_type = other DNA

SEQ ID NO: 378 - DNA, 31 bp, synthetic construct
  misc_feature 1..31: note = source = /note="Description of Artificial Sequence: Synthetic oligonucleotide"
  source 1..31: mol_type = other DNA
ttgaacttctttgttgtcttcgacatgggta

SEQ ID NO: 379 - DNA, 36 bp, synthetic construct; identical to SEQ ID NO: 189
  misc_feature 1..36: note = source = /note="Description of Artificial Sequence: Synthetic oligonucleotide"
  source 1..36: mol_type = other DNA

SEQ ID NO: 380 - DNA, 31 bp, synthetic construct
  misc_feature 1..31: note = source = /note="Description of Artificial Sequence: Synthetic oligonucleotide"
  source 1..31: mol_type = other DNA
aaacgtaatgcggggtgcatttcgctgattt

SEQ ID NO: 381 - DNA, 36 bp, synthetic construct; identical to SEQ ID NO: 189
  misc_feature 1..36: note = source = /note="Description of Artificial Sequence: Synthetic oligonucleotide"
  source 1..36: mol_type = other DNA

SEQ ID NO: 382 - DNA, 31 bp, synthetic construct; identical to SEQ ID NO: 296
  misc_feature 1..31: note = source = /note="Description of Artificial Sequence: Synthetic oligonucleotide"
  source 1..31: mol_type = other DNA

SEQ ID NO: 383 - DNA, 36 bp, synthetic construct; identical to SEQ ID NO: 189
  misc_feature 1..36: note = source = /note="Description of Artificial Sequence: Synthetic oligonucleotide"
  source 1..36: mol_type = other DNA

SEQ ID NO: 384 - DNA, 31 bp, synthetic construct
  misc_feature 1..31: note = source = /note="Description of Artificial Sequence: Synthetic oligonucleotide"
  source 1..31: mol_type = other DNA
tccaccaaacgtaatgcggggtgcatttcgc

SEQ ID NO: 385 - DNA, 36 bp, synthetic construct; identical to SEQ ID NO: 189
  misc_feature 1..36: note = source = /note="Description of Artificial Sequence: Synthetic oligonucleotide"
  source 1..36: mol_type = other DNA

SEQ ID NO: 386 - DNA, 31 bp, synthetic construct
  misc_feature 1..31: note = source = /note="Description of Artificial Sequence: Synthetic oligonucleotide"
  source 1..31: mol_type = other DNA
gggtccaccaaacgtaatgcggggtgcattt

SEQ ID NO: 387 - DNA, 36 bp, synthetic construct; identical to SEQ ID NO: 189
  misc_feature 1..36: note = source = /note="Description of Artificial Sequence: Synthetic oligonucleotide"
  source 1..36: mol_type = other DNA

SEQ ID NO: 388 - DNA, 31 bp, synthetic construct
  misc_feature 1..31: note = source = /note="Description of Artificial Sequence: Synthetic oligonucleotide"
  source 1..31: mol_type = other DNA
tgagggtccaccaaacgtaatgcggggtgca

SEQ ID NO: 389 - DNA, 36 bp, synthetic construct; identical to SEQ ID NO: 189
  misc_feature 1..36: note = source = /note="Description of Artificial Sequence: Synthetic oligonucleotide"
  source 1..36: mol_type = other DNA

SEQ ID NO: 390 - DNA, 31 bp, synthetic construct
  misc_feature 1..31: note = source = /note="Description of Artificial Sequence: Synthetic oligonucleotide"
  source 1..31: mol_type = other DNA
atctgagggtccaccaaacgtaatgcggggt

SEQ ID NO: 391 - DNA, 36 bp, synthetic construct; identical to SEQ ID NO: 189
  misc_feature 1..36: note = source = /note="Description of Artificial Sequence: Synthetic oligonucleotide"
  source 1..36: mol_type = other DNA

SEQ ID NO: 392 - DNA, 31 bp, synthetic construct
  misc_feature 1..31: note = source = /note="Description of Artificial Sequence: Synthetic oligonucleotide"
  source 1..31: mol_type = other DNA
tgaatctgagggtccaccaaacgtaatgcgg

SEQ ID NO: 393 - DNA, 36 bp, synthetic construct; identical to SEQ ID NO: 189
  misc_feature 1..36: note = source = /note="Description of Artificial Sequence: Synthetic oligonucleotide"
  source 1..36: mol_type = other DNA

SEQ ID NO: 394 - DNA, 31 bp, synthetic construct
  misc_feature 1..31: note = source = /note="Description of Artificial Sequence: Synthetic oligonucleotide"
  source 1..31: mol_type = other DNA
agttgaatctgagggtccaccaaacgtaatg

SEQ ID NO: 395 - DNA, 36 bp, synthetic construct; identical to SEQ ID NO: 189
  misc_feature 1..36: note = source = /note="Description of Artificial Sequence: Synthetic oligonucleotide"
  source 1..36: mol_type = other DNA

SEQ ID NO: 396 - DNA, 31 bp, synthetic construct
  misc_feature 1..31: note = source = /note="Description of Artificial Sequence: Synthetic oligonucleotide"
  source 1..31: mol_type = other DNA
gccagttgaatctgagggtccaccaaacgta

SEQ ID NO: 397 - DNA, 36 bp, synthetic construct; identical to SEQ ID NO: 189
  misc_feature 1..36: note = source = /note="Description of Artificial Sequence: Synthetic oligonucleotide"
  source 1..36: mol_type = other DNA

SEQ ID NO: 398 - DNA, 31 bp, synthetic construct
  misc_feature 1..31: note = source = /note="Description of Artificial Sequence: Synthetic oligonucleotide"
  source 1..31: mol_type = other DNA
actgccagttgaatctgagggtccaccaaac

SEQ ID NO: 399 - DNA, 36 bp, synthetic construct; identical to SEQ ID NO: 189
  misc_feature 1..36: note = source = /note="Description of Artificial Sequence: Synthetic oligonucleotide"
  source 1..36: mol_type = other DNA

SEQ ID NO: 400 - DNA, 31 bp, synthetic construct
  misc_feature 1..31: note = source = /note="Description of Artificial Sequence: Synthetic oligonucleotide"
  source 1..31: mol_type = other DNA
gttactgccagttgaatctgagggtccacca

SEQ ID NO: 401 - DNA, 31 bp, synthetic construct
  misc_feature 1..31: note = source = /note="Description of Artificial Sequence: Synthetic oligonucleotide"
  source 1..31: mol_type = other DNA
gcagatcagggcaaacagaactttgactccc

SEQ ID NO: 402 - DNA, 31 bp, synthetic construct
  misc_feature 1..31: note = source = /note="Description of Artificial Sequence: Synthetic oligonucleotide"
  source 1..31: mol_type = other DNA
cgatgttgaagtcttcgttgttctcggtggg

SEQ ID NO: 403 - DNA, 31 bp, synthetic construct
  misc_feature 1..31: note = source = /note="Description of Artificial Sequence: Synthetic oligonucleotide"
  source 1..31: mol_type = other DNA
gcaacttcccgcggtcagcatcgagatccgt

SEQ ID NO: 404 - DNA, 31 bp, synthetic construct
  misc_feature 1..31: note = source = /note="Description of Artificial Sequence: Synthetic oligonucleotide"
  source 1..31: mol_type = other DNA
tgcagccagctttccgggcattggcttccat

SEQ ID NO: 405 - DNA, 31 bp, synthetic construct
  misc_feature 1..31: note = source = /note="Description of Artificial Sequence: Synthetic oligonucleotide"
  source 1..31: mol_type = other DNA
tgaacttcttcatcttgggcgtgcacttgat

SEQ ID NO: 406 - DNA, 31 bp, synthetic construct
  misc_feature 1..31: note = source = /note="Description of Artificial Sequence: Synthetic oligonucleotide"
  source 1..31: mol_type = other DNA
gaatgtcgacgatcgcctcgcctatgccgcc

SEQ ID NO: 407 - DNA, 31 bp, synthetic construct
  misc_feature 1..31: note = source = /note="Description of Artificial Sequence: Synthetic oligonucleotide"
  source 1..31: mol_type = other DNA
atgaactgctccatgggctccaagtccttga

SEQ ID NO: 408 - DNA, 31 bp, synthetic construct
  misc_feature 1..31: note = source = /note="Description of Artificial Sequence: Synthetic oligonucleotide"
  source 1..31: mol_type = other DNA
gcagccagttgtgcagtccacacacagatcg

SEQ ID NO: 409 - DNA, 31 bp, synthetic construct
  misc_feature 1..31: note = source = /note="Description of Artificial Sequence: Synthetic oligonucleotide"
  source 1..31: mol_type = other DNA
gcgttgcggcagccacttcttgagcaggtca

SEQ ID NO: 410 - DNA, 31 bp, synthetic construct
  misc_feature 1..31: note = source = /note="Description of Artificial Sequence: Synthetic oligonucleotide"
  source 1..31: mol_type = other DNA
tgtccacctggccctggatcttgctggcaaa

SEQ ID NO: 411 - DNA, 31 bp, synthetic construct
  misc_feature 1..31: note = source = /note="Description of Artificial Sequence: Synthetic oligonucleotide"
  source 1..31: mol_type = other DNA
ggtaatgcctggcttgtcgacgcatagtctg

SEQ ID NO: 412 - DNA, 31 bp, synthetic construct
  misc_feature 1..31: note = source = /note="Description of Artificial Sequence: Synthetic oligonucleotide"
  source 1..31: mol_type = other DNA
gggaaccttggccgttataaagtctgaccag

SEQ ID NO: 413 - DNA, 31 bp, synthetic construct
  misc_feature 1..31: note = source = /note="Description of Artificial Sequence: Synthetic oligonucleotide"
  source 1..31: mol_type = other DNA
tataatggtgaatatcttcaaatgatttagt

SEQ ID NO: 414 - DNA, 31 bp, synthetic construct
  misc_feature 1..31: note = source = /note="Description of Artificial Sequence: Synthetic oligonucleotide"
  source 1..31: mol_type = other DNA
atgtatagaaggcatcatcaacaccctgtct

SEQ ID NO: 415 - DNA, 31 bp, synthetic construct
  misc_feature 1..31: note = source = /note="Description of Artificial Sequence: Synthetic oligonucleotide"
  source 1..31: mol_type = other DNA
ggttaaaaatttacagattgtgctgagcttg

SEQ ID NO: 416 - DNA, 31 bp, synthetic construct
  misc_feature 1..31: note = source = /note="Description of Artificial Sequence: Synthetic oligonucleotide"
  source 1..31: mol_type = other DNA
gtagatgctctttcctcctgtgccatctccc

SEQ ID NO: 417 - DNA, 31 bp, synthetic construct
  misc_feature 1..31: note = source = /note="Description of Artificial Sequence: Synthetic oligonucleotide"
  source 1..31: mol_type = other DNA
cagtttgaagttctcatcggggaagcgctca

SEQ ID NO: 418 - DNA, 31 bp, synthetic construct
  misc_feature 1..31: note = source = /note="Description of Artificial Sequence: Synthetic oligonucleotide"
  source 1..31: mol_type = other DNA
cagtgttggtaggagtttgttacaaaagtga

SEQ ID NO: 419 - DNA, 31 bp, synthetic construct
  misc_feature 1..31: note = source = /note="Description of Artificial Sequence: Synthetic oligonucleotide"
  source 1..31: mol_type = other DNA
cttggccaagtctgttatgttcacctgaaaa

SEQ ID NO: 420 - DNA, 31 bp, synthetic construct
  misc_feature 1..31: note = source = /note="Description of Artificial Sequence: Synthetic oligonucleotide"
  source 1..31: mol_type = other DNA
caaaatgtactcagcttcaatcacaaatacg

SEQ ID NO: 421 - DNA, 31 bp, synthetic construct
  misc_feature 1..31: note = source = /note="Description of Artificial Sequence: Synthetic oligonucleotide"
  source 1..31: mol_type = other DNA
ggttatagcttgacaagcaattaactttaaa

SEQ ID NO: 422 - DNA, 31 bp, synthetic construct
  misc_feature 1..31: note = source = /note="Description of Artificial Sequence: Synthetic oligonucleotide"
  source 1..31: mol_type = other DNA
atgataatgcaatagcaggacaggatgacaa

SEQ ID NO: 423 - DNA, 31 bp, synthetic construct; identical to SEQ ID NO: 411
  misc_feature 1..31: note = source = /note="Description of Artificial Sequence: Synthetic oligonucleotide"
  source 1..31: mol_type = other DNA

SEQ ID NO: 424 - DNA, 31 bp, synthetic construct; identical to SEQ ID NO: 412
  misc_feature 1..31: note = source = /note="Description of Artificial Sequence: Synthetic oligonucleotide"
  source 1..31: mol_type = other DNA

SEQ ID NO: 425 - DNA, 50 bp, synthetic construct
  misc_feature 1..50: note = source = /note="Description of Artificial Sequence: Synthetic oligonucleotide"
  source 1..50: mol_type = other DNA
catcctgcggcctctactctgcattcaattacatactgacacattcggca

SEQ ID NO: 426 - DNA, 50 bp, synthetic construct
  misc_feature 1..50: note = source = /note="Description of Artificial Sequence: Synthetic oligonucleotide"
  source 1..50: mol_type = other DNA
accatcctgcggcctctactctgcattcaattacatactgacacattcgg

SEQ ID NO: 427 - DNA, 50 bp, synthetic construct
  misc_feature 1..50: note = source = /note="Description of Artificial Sequence: Synthetic oligonucleotide"
  source 1..50: mol_type = other DNA
aaaccatcctgcggcctctactctgcattcaattacatactgacacattc

SEQ ID NO: 428 - DNA, 50 bp, synthetic construct
  misc_feature 1..50: note = source = /note="Description of Artificial Sequence: Synthetic oligonucleotide"
  source 1..50: mol_type = other DNA
ctaaaccatcctgcggcctctactctgcattcaattacatactgacacat

SEQ ID NO: 429 - DNA, 50 bp, synthetic construct
  misc_feature 1..50: note = source = /note="Description of Artificial Sequence: Synthetic oligonucleotide"
  source 1..50: mol_type = other DNA
ttctaaaccatcctgcggcctctactctgcattcaattacatactgacac

SEQ ID NO: 430 - DNA, 50 bp, synthetic construct
  misc_feature 1..50: note = source = /note="Description of Artificial Sequence: Synthetic oligonucleotide"
  source 1..50: mol_type = other DNA
tgttctaaaccatcctgcggcctctactctgcattcaattacatactgac

SEQ ID NO: 431 - DNA, 50 bp, synthetic construct
  misc_feature 1..50: note = source = /note="Description of Artificial Sequence: Synthetic oligonucleotide"
  source 1..50: mol_type = other DNA
aatgttctaaaccatcctgcggcctctactctgcattcaattacatactg

SEQ ID NO: 432 - DNA, 50 bp, synthetic construct
  misc_feature 1..50: note = source = /note="Description of Artificial Sequence: Synthetic oligonucleotide"
  source 1..50: mol_type = other DNA
agaatgttctaaaccatcctgcggcctctactctgcattcaattacatac

SEQ ID NO: 433 - DNA, 50 bp, synthetic construct
  misc_feature 1..50: note = source = /note="Description of Artificial Sequence: Synthetic oligonucleotide"
  source 1..50: mol_type = other DNA
atagaatgttctaaaccatcctgcggcctctactctgcattcaattacat

SEQ ID NO: 434 - DNA, 50 bp, synthetic construct
  misc_feature 1..50: note = source = /note="Description of Artificial Sequence: Synthetic oligonucleotide"
  source 1..50: mol_type = other DNA
ccatagaatgttctaaaccatcctgcggcctctactctgcattcaattac

SEQ ID NO: 435 - DNA, 50 bp, synthetic construct
  misc_feature 1..50: note = source = /note="Description of Artificial Sequence: Synthetic oligonucleotide"
  source 1..50: mol_type = other DNA
ttccatagaatgttctaaaccatcctgcggcctctactctgcattcaatt

SEQ ID NO: 436 - DNA, 50 bp, synthetic construct
  misc_feature 1..50: note = source = /note="Description of Artificial Sequence: Synthetic oligonucleotide"
  source 1..50: mol_type = other DNA
ctttccatagaatgttctaaaccatcctgcggcctctactctgcattcaa

SEQ ID NO: 437 - DNA, 50 bp, synthetic construct
  misc_feature 1..50: note = source = /note="Description of Artificial Sequence: Synthetic oligonucleotide"
  source 1..50: mol_type = other DNA
ctctttccatagaatgttctaaaccatcctgcggcctctactctgcattc

SEQ ID NO: 438 - DNA, 50 bp, synthetic construct
  misc_feature 1..50: note = source = /note="Description of Artificial Sequence: Synthetic oligonucleotide"
  source 1..50: mol_type = other DNA
atctctttccatagaatgttctaaaccatcctgcggcctctactctgcat

SEQ ID NO: 439 - DNA, 50 bp, synthetic construct
  misc_feature 1..50: note = source = /note="Description of Artificial Sequence: Synthetic oligonucleotide"
  source 1..50: mol_type = other DNA
gaatctctttccatagaatgttctaaaccatcctgcggcctctactctgc

SEQ ID NO: 440 - DNA, 50 bp, synthetic construct
  misc_feature 1..50: note = source = /note="Description of Artificial Sequence: Synthetic oligonucleotide"
  source 1..50: mol_type = other DNA
tggaatctctttccatagaatgttctaaaccatcctgcggcctctactct

SEQ ID NO: 441 - DNA, 50 bp, synthetic construct
  misc_feature 1..50: note = source = /note="Description of Artificial Sequence: Synthetic oligonucleotide"
  source 1..50: mol_type = other DNA
actggaatctctttccatagaatgttctaaaccatcctgcggcctctact

SEQ ID NO: 442 - DNA, 50 bp, synthetic construct
  misc_feature 1..50: note = source = /note="Description of Artificial Sequence: Synthetic oligonucleotide"
  source 1..50: mol_type = other DNA
gaactggaatctctttccatagaatgttctaaaccatcctgcggcctcta

SEQ ID NO: 443 - DNA, 50 bp, synthetic construct
  misc_feature 1..50: note = source = /note="Description of Artificial Sequence: Synthetic oligonucleotide"
  source 1..50: mol_type = other DNA
tggaactggaatctctttccatagaatgttctaaaccatcctgcggcctc

SEQ ID NO: 444 - DNA, 50 bp, synthetic construct
  misc_feature 1..50: note = source = /note="Description of Artificial Sequence: Synthetic oligonucleotide"
  source 1..50: mol_type = other DNA
cctggaactggaatctctttccatagaatgttctaaaccatcctgcggcc

SEQ ID NO: 445 - DNA, 50 bp, synthetic construct
  misc_feature 1..50: note = source = /note="Description of Artificial Sequence: Synthetic oligonucleotide"
  source 1..50: mol_type = other DNA
ttcctggaactggaatctctttccatagaatgttctaaaccatcctgcgg

SEQ ID NO: 446 - DNA, 50 bp, synthetic construct
  misc_feature 1..50: note = source = /note="Description of Artificial Sequence: Synthetic oligonucleotide"
  source 1..50: mol_type = other DNA
ggttcctggaactggaatctctttccatagaatgttctaaaccatcctgc

SEQ ID NO: 447 - DNA, 28 bp, synthetic construct
  misc_feature 1..28: note = source = /note="Description of Artificial Sequence: Synthetic oligonucleotide"
  source 1..28: mol_type = other DNA
acccaggaatctcaggaatgtcgacgat

SEQ ID NO: 448 - DNA, 30 bp, synthetic construct
  misc_feature 1..30: note = source = /note="Description of Artificial Sequence: Synthetic oligonucleotide"
  source 1..30: mol_type = other DNA
gggcattggcttccatctctttgagcacct

SEQ ID NO: 449 - DNA, 28 bp, synthetic construct
  misc_feature 1..28: note = source = /note="Description of Artificial Sequence: Synthetic oligonucleotide"
  source 1..28: mol_type = other DNA
acccaggaatctcaggaatgtcgacgat

SEQ ID NO: 450 - DNA, 48 bp, synthetic construct
  misc_feature 1..48: note = source = /note="Description of Artificial Sequence: Synthetic oligonucleotide"
  source 1..48: mol_type = other DNA
aaagttctgtttgccctgatcctcgaggatcagggcaaacagaacttt

SEQ ID NO: 451 - protein, 45 aa, unidentified
  REGION 1..45: note = source = /note="Description of Unknown: Candidatus Poribacteria bacterium sequence"
RDMATSAHTTIDKETKRTKENHLINLEVVRIEDNRYLKGSVEMIN

SEQ ID NO: 452 - protein, 44 aa, unidentified
  REGION 1..44: note = source = /note="Description of Unknown: Methanomicrobia archaeon sequence"
DQVRTSAHNKINRATGRVEKDLLFSYELGANTVPLEGTIERVNP

SEQ ID NO: 453 - protein, 44 aa, unidentified
  REGION 1..44: note = source = /note="Description of Unknown: Methanophagales archaeon sequence"
DMVRTSTHNKISRATGRVEKDHLFSYELGANTTPFEGMIEPVNH

SEQ ID NO: 454 - protein, 43 aa, unidentified
  REGION 1..43: note = source = /note="Description of Unknown: Deltaproteobacteria bacterium sequence"
GTTRVSLHNRVDPTTGRVPEETFFSFEVGSAEKFTGRIERSGP

SEQ ID NO: 455 - protein, 43 aa, unidentified
  REGION 1..43: note = source = /note="Description of Unknown: Firmicutes bacterium sequence"
EPIRISTHNRIDPATGRVPKDLLFTFEMGSPAVFRGYIERIGI

SEQ ID NO: 456 - protein, 44 aa, unidentified
  REGION 1..44: note = source = /note="Description of Unknown: Ktedonosporobacter rubrisoli sequence"
LQTFLYTQARLDRPTRTAVPGALYSSEFGLREITFKGSVTGWLE

SEQ ID NO: 457 - protein, 44 aa, unidentified
  REGION 1..44: note = source = /note="Description of Unknown: Chloroflexi bacterium sequence"
NQKDVYTQVRLDRPTRTAVEGALYTSEFGTRGLTFHGDITGSLT

SEQ ID NO: 458 - protein, 44 aa, unidentified
  REGION 1..44: note = source = /note="Description of Unknown: ANME-2 cluster archaeon sequence"
LFLNTRTGTKIDRRTGTVQEDHLFTTESGIKGITLTSKIYGRGN

SEQ ID NO: 459 - protein, 44 aa, unidentified
  REGION 1..44: note = source = /note="Description of Unknown: Anaerolineaceae bacterium sequence"
YGQSIRSNNRIDPKTRTAANDHLFFTECVDPDTYFIGEIRSSSN

SEQ ID NO: 460 - protein, 44 aa, unidentified
  REGION 1..44: note = source = /note="Description of Unknown: Ornatilinea apprima sequence"
YGRLLRTGIGIDRKLRVVVPDRLFTNECVGTKIPFTGRIIGEFP

SEQ ID NO: 461 - protein, 44 aa, unidentified
  REGION 1..44: note = source = /note="Description of Unknown: HreCas7-11 sequence"
EIAVSRVVNRVDPNSGKAKDFFRVWEIDHKLCPNFLGKMSISLS

SEQ ID NO: 462 - protein, 44 aa, unidentified
  REGION 1..44: note = source = /note="Description of Unknown: CmaCas7-11 sequence"
DIVQKRILNRVDQTCGKAKDFFKVCEVDHIACPTLNGIIRINDE

SEQ ID NO: 463 - protein, 42 aa, unidentified
  REGION 1..42: note = source = /note="Description of Unknown: HvmCas7-11 sequence"
ETAIERIVNRVDPSSGKAKDYMRIWEIDPLVCSQFNGIITIN

SEQ ID NO: 464 - protein, 42 aa, unidentified
  REGION 1..42: note = source = /note="Description of Unknown: SmCas7-11 sequence"
EIGSRRTINRVDDETGKAHDFFSIWEVDAVREFQGEIVLAAD

SEQ ID NO: 465 - protein, 42 aa, unidentified
  REGION 1..42: note = source = /note="Description of Unknown: OmCas7-11 sequence"
EIGRLRTLNRIDRLTTKAQDFFRIYEVDQVRDFFGTITLAGD

SEQ ID NO: 466 - protein, 44 aa, unidentified
  REGION 1..44: note = source = /note="Description of Unknown: DisCas7-11 sequence"
AIGSQRVLNRVDFKSGKAHDFFKAYEVDHTRFPRFEGEITIDNK

SEQ ID NO: 467 - protein, 44 aa, unidentified
  REGION 1..44: note = source = /note="Description of Unknown: DsbaCas7-11 sequence"
EIAVRRVVNRVDYASGKAHDFFRIFEVDHIAFPCFHGEIAFGEN

SEQ ID NO: 468 - protein, 44 aa, unidentified
  REGION 1..44: note = source = /note="Description of Unknown: SstCas7-11 sequence"
DIGTQRTLNRVDYATGKAHDFFKVWEIDHSLLSVFQGKISIADN

SEQ ID NO: 469 - protein, 44 aa, unidentified
  REGION 1..44: note = source = /note="Description of Unknown: CsbCas7-11 sequence"
DIASGRILNRVDFDTGKAKDYFRTWEADYETYGTYTGRITLRNE

SEQ ID NO: 470 - protein, 44 aa, unidentified
  REGION 1..44: note = source = /note="Description of Unknown: CjcCas7-11 sequence"
DIASERILNRVDYTTGKAHDYFKVWEVDDDQWWQFTGTITMHDD

SEQ ID NO: 471 - protein, 44 aa, unidentified
  REGION 1..44: note = source = /note="Description of Unknown: CbfCas7-11 sequence"
DVCKERVLNRVDFRTGKAQDYFRVFEIDHEDWGVYTGEITITEP

SEQ ID NO: 472 - protein, 44 aa, unidentified
  REGION 1..44: note = source = /note="Description of Unknown: HvsCas7-11 sequence"
ELAPKRALNRIEQYTGKAQDYFTVYEALNKEFWTFKGRIRIKED

SEQ ID NO: 473 - protein, 44 aa, unidentified
  REGION 1..44: note = source = /note="Description of Unknown: HsmCas7-11 sequence"
EIGIRRILNRVDFTTGKAQDYFYVWEVEHSMCPKFQGTVKINED

SEQ ID NO: 474 - protein, 42 aa, unidentified
  REGION 1..42: note = source = /note="Description of Unknown: FmCas7-11 sequence"
DIAQKRTSNRVDFFSKKAHDHYGVWEVTAVKNLLGYIYISDA

SEQ ID NO: 475 - protein, 44 aa, unidentified
  REGION 1..44: note = source = /note="Description of Unknown: DpbaCas7-11 sequence"
QLIRPRILNRMDPGTSKARDYFRVFEIENQLCSQFRGWIWLSGD

SEQ ID NO: 476 - protein, 30 aa, unidentified
  REGION 1..30: note = source = /note="Description of Unknown: WmCas7x3 sequence"
QRITPDAPDADPYWQADIALDADGRPYLPG

SEQ ID NO: 477 - protein, 35 aa, unidentified
  REGION 1..35: note = source = /note="Description of Unknown: NisCas7x3 sequence"
EEMDKQSGESVDKKQNNSWIQAIALDLNKKPYIPG

SEQ ID NO: 478 - protein, 28 aa, unidentified
  REGION 1..28: note = source = /note="Description of Unknown: GwCas7x3 sequence"
EKERDENKETRWLEAVALDHKGQPYIPG

SEQ ID NO: 479 - protein, 32 aa, unidentified
  REGION 1..32: note = source = /note="Description of Unknown: HvmCas7x3 sequence"
VTRPELTVADRDELVDINAVVTDYTGKPYLPG

SEQ ID NO: 480 - protein, 35 aa, unidentified
  REGION 1..35: note = source = /note="Description of Unknown: DsbCas7x3 sequence"
TDRPGLEIEQKDGSKVKAEINAFIKDSNGKPYLPG

SEQ ID NO: 481 - protein, 32 aa, unidentified
  REGION 1..32: note = source = /note="Description of Unknown: GamCas7x3 sequence"
IPVTIKDTQGKNREVEVNSVITGKAALPIIPG

SEQ ID NO: 482 - protein, 31 aa, unidentified
  REGION 1..31: note = source = /note="Description of Unknown: DesCas7x3 sequence"
VEHDLIKNDDGTPVQINALITGAGGLPIIPG

SEQ ID NO: 483 - protein, 32 aa, unidentified
  REGION 1..32: note = source = /note="Description of Unknown: GabCas7x3 sequence"
AEIEINDSQGDRRQVQANAIIRGKDDKPIIPG

SEQ ID NO: 484 - protein, 31 aa, unidentified
  REGION 1..31: note = source = /note="Description of Unknown: MetCas7x3 sequence"
CEHDDVKNNDGEPVKINACIKGSKGRPIIPG

SEQ ID NO: 485 - protein, 31 aa, unidentified
  REGION 1..31: note = source = /note="Description of Unknown: MebCas7x3 sequence"
CYRPELTNADQKPVDINACIKGANNLPIIPG

SEQ ID NO: 486 - protein, 29 aa, unidentified
  REGION 1..29: note = source = /note="Description of Unknown: GwCas7-11 sequence"
EGQKQTDDQAESLHLRTLRDGHGRFRIPF

SEQ ID NO: 487 - protein, 21 aa, unidentified
  REGION 1..21: note = source = /note="Description of Unknown: HreCas7-11 sequence"
SEGQTSQAIILCPDGSYRLPR

SEQ ID NO: 488 - protein, 21 aa, unidentified
  REGION 1..21: note = source = /note="Description of Unknown: CmaCas7-11 sequence"
KTSHTRSTIFLNMNGQFCIPR

SEQ ID NO: 489 - protein, 21 aa, unidentified
  REGION 1..21: note = source = /note="Description of Unknown: HvmCas7-11 sequence"
QEGQTSATILLNRDGYFRLPR

SEQ ID NO: 490 - protein, 23 aa, unidentified
  REGION 1..23: note = source = /note="Description of Unknown: SmCas7-11 sequence"
PIDDDQTSSRTLVDRDGRYRLPR

SEQ ID NO: 491 - protein, 22 aa, unidentified
  REGION 1..22: note = source = /note="Description of Unknown: OmCas7-11 sequence"
GSHDHTSRKILLTRDFYYRLPR

SEQ ID NO: 492 - protein, 22 aa, unidentified
  REGION 1..22: note = source = /note="Description of Unknown: DisCas7-11 sequence"
EDAKQTDLQVLLTPDNKYRLPR

SEQ ID NO: 493 - protein, 22 aa, unidentified
  REGION 1..22: note = source = /note="Description of Unknown: DsbaCas7-11 sequence"
KEKKQTDLMLLLDGQNHYRIPR

SEQ ID NO: 494 - protein, 22 aa, unidentified
  REGION 1..22: note = source = /note="Description of Unknown: SstCas7-11 sequence"
EDAKHTNLKVLLDRQNRYRLPR

SEQ ID NO: 495 - protein, 22 aa, unidentified
  REGION 1..22: note = source = /note="Description of Unknown: SybCas7-11 sequence"
GAGGHTDLSILLGKDGHYRVPR

SEQ ID NO: 496 - protein, 37 aa, unidentified
  REGION 1..37: note = source = /note="Description of Unknown: CsbCas7-11 sequence"
SDSIPGKEKKSEDSLVINEHTSFNILLDKENRYRIPR

SEQ ID NO: 497 - protein, 43 aa, unidentified
  REGION 1..43: note = source = /note="Description of Unknown: CjcCas7-11 sequence"
FDSTQDDLDLVPDIVNTDEKLEANEQTSFRILMDKKGRYRIPR

SEQ ID NO: 498 - protein, 25 aa, unidentified
  REGION 1..25: note = source = /note="Description of Unknown: CbfCas7-11 sequence"
EQTEEVEHTSLRLVMDKKGRFRIPR

SEQ ID NO: 499 - protein, 21 aa, unidentified
  REGION 1..21: note = source = /note="Description of Unknown: HvsCas7-11 sequence"
DEEKAEGAILLTPDNRFRLPR

SEQ ID NO: 500 - protein, 22 aa, unidentified
  REGION 1..22: note = source = /note="Description of Unknown: HsmCas7-11 sequence"
KTEEQIDMQILLTKDGRYRLPR

SEQ ID NO: 501 - protein, 22 aa, unidentified
  REGION 1..22: note = source = /note="Description of Unknown: FmCas7-11 sequence"
QAGKQTSMPTLQDSNDHFRLPR

SEQ ID NO: 502 - protein, 22 aa, unidentified
  REGION 1..22: note = source = /note="Description of Unknown: DpbaCas7-11 sequence"
ATETHTDLPILLTSDRHFRIPR

SEQ ID NO: 503 - protein, 33 aa, unidentified
  REGION 1..33: note = source = /note="Description of Unknown: WmCas7x3 sequence"
HRKPQDNAPDGTPRQRGDRALLPGASLRGRLRS

SEQ ID NO: 504 - protein, 34 aa, unidentified
  REGION 1..34: note = source = /note="Description of Unknown: NisCas7x3 sequence"
KKDEAKNEADAKPRTNHQGQVILPASSLRGRLRA

SEQ ID NO: 505 - protein, 30 aa, unidentified
  REGION 1..30: note = source = /note="Description of Unknown: GwCas7x3 sequence"
KADDADAVPRRTHDDKIVLPASSLRGRLRT

SEQ ID NO: 506 - protein, 34 aa, unidentified
  REGION 1..34: note = source = /note="Description of Unknown: HvmCas7x3 sequence"
KKKRRSNTPNLRPLRDAIGRPCLPESSVRGALRA

SEQ ID NO: 507 - protein, 32 aa, unidentified
  REGION 1..32: note = source = /note="Description of Unknown: DsbCas7x3 sequence"
KKRKEDIDHQPLRDSAGNARLPAKSIRGAMRS

SEQ ID NO: 508 - protein, 34 aa, unidentified
  REGION 1..34: note = source = /note="Description of Unknown: GamCas7x3 sequence"
DKCKAEDTPDIYPLEEKNGVPAFPVRSFRGAIRS

SEQ ID NO: 509 - protein, 34 aa, unidentified
  REGION 1..34: note = source = /note="Description of Unknown: DesCas7x3 sequence"
NTPDITQAPDMVPLVDEDGNPMLPASSFRGALRA

SEQ ID NO: 510 - protein, 34 aa, unidentified
  REGION 1..34: note = source = /note="Description of Unknown: GabCas7x3 sequence"
SKGDGDQPAVHQPLTDRSNNPILPARSFRGAIRA

SEQ ID NO: 511 - protein, 34 aa, unidentified
  REGION 1..34: note = source = /note="Description of Unknown: MetCas7x3 sequence"
LSSNENAKTDHYPLLDKNRNPYLPVSSFRGVLRS

SEQ ID NO: 512 - protein, 34 aa, unidentified
  REGION 1..34: note = source = /note="Description of Unknown: MebCas7x3 sequence"
LEADPKTKIDHYPLLDNHKKPRLPSASIRGVLRS

SEQ ID NO: 513 - protein, 48 aa, unidentified
  REGION 1..48: note = source = /note="Description of Unknown: GwCas7-11 sequence"
ESDAPDNVAYKKPVVQYDETGRLRTTDPGPVEMLTCLKGEGVRGVVAY

SEQ ID NO: 514 - protein, 42 aa, unidentified
  REGION 1..42: note = source = /note="Description of Unknown: HreCas7-11 sequence"
DHDNKDAVMVQKTVLFVDESGNYSQMPHHFLKGSGIRGACRF

SEQ ID NO: 515 - protein, 47 aa, unidentified
  REGION 1..47: note = source = /note="Description of Unknown: CmaCas7-11 sequence"
DNRNPDAVMVKKTILVYEQDSSTHKNVPKEVPKYFIKSETIRGLLRS

SEQ ID NO: 516 - protein, 44 aa, unidentified
  REGION 1..44: note = source = /note="Description of Unknown: HvmCas7-11 sequence"
DPRNTDAIMVRKTVFCPDPNAKNRPAPATVYMIKGESIRGILRS

SEQ ID NO: 517 - protein, 37 aa, unidentified
  REGION 1..37: note = source = /note="Description of Unknown: SmCas7-11 sequence"
NSDTADIISFRRTVVDNGEVLREPVLRGEGLRGLLRT

SEQ ID NO: 518 - protein, 37 aa, unidentified
  REGION 1..37: note = source = /note="Description of Unknown: OmCas7-11 sequence"
RHEEDSVYFQKRIFTSDGRVVLVPALRGEGLRGLLRT

SEQ ID NO: 519 - protein, 37 aa, unidentified
  REGION 1..37: note = source = /note="Description of Unknown: DisCas7-11 sequence"
DKRGTDVVTFVKYKAEGEEAKPVCAYKAESFRGVIRS

SEQ ID NO: 520 - protein, 35 aa, unidentified
  REGION 1..35: note = source = /note="Description of Unknown: DsbaCas7-11 sequence"
TEDVADIVSFKKYTQGGEKIIYAYKSESFRGVVRT

SEQ ID NO: 521 - protein, 35 aa, unidentified
  REGION 1..35: note = source = /note="Description of Unknown: SstCas7-11 sequence"
EKDGSDIVSFRKYADDSGKEVYAYKAESFRGVVRA

SEQ ID NO: 522 - protein, 39 aa, unidentified
  REGION 1..39: note = source = /note="Description of Unknown: SybCas7-11 sequence"
ERIGFDNIAYEKRRYNGETNTTESIPAVKGETFRGIVRT

SEQ ID NO: 523 - protein, 41 aa, unidentified
  REGION 1..41: note = source = /note="Description of Unknown: CsbCas7-11 sequence"
EPGNRDAIAYKKRVYNDGNNAIEPEPRFAVKSETHRGIFRT

SEQ ID NO: 524 - protein, 37 aa, unidentified
  REGION 1..37: note = source = /note="Description of Unknown: CjcCas7-11 sequence"
NKDNIDCIAYEKRKWENGGIKFVPTIKGETIRGIVRM

SEQ ID NO: 525 - protein, 37 aa, unidentified
  REGION 1..37: note = source = /note="Description of Unknown: CbfCas7-11 sequence"
DPNNVDAIVFEKMKLDGDQVKYLPAIKGETIRGIVRT

SEQ ID NO: 526 - protein, 37 aa, unidentified
  REGION 1..37: note = source = /note="Description of Unknown: HvsCas7-11 sequence"
EGKAPDAVFFKKYVFENGKIEEKPCFKAESIRGIFRT

SEQ ID NO: 527 - protein, 35 aa, unidentified
  REGION 1..35: note = source = /note="Description of Unknown: HsmCas7-11 sequence"
DSSNTDLVTFKKYKLEESKEVFAIKGESIRGVFRT

SEQ ID NO: 528 - protein, 38 aa, unidentified
  REGION 1..38: note = source = /note="Description of Unknown: FmCas7-11 sequence"
HGNTDSVFYKKPILKSGEKEPSYQWAIKSDTVRGLIRS

SEQ ID NO: 529 - protein, 34 aa, unidentified
  REGION 1..34: note = source = /note="Description of Unknown: DpbaCas7-11 sequence"
RNESDAVFYQKSVAGEKGPVYALKGEGLRGIVSS

SEQ ID NO: 530 - protein, 43 aa, unidentified
  REGION 1..43: note = source = /note="Description of Unknown: WmCas7x3 sequence"
PATLVDHDMLAIDRFTGGGKDGAKFKLRYAECPTLEGQLSLDL

SEQ ID NO: 531 - protein, 43 aa, unidentified
  REGION 1..43: note = source = /note="Description of Unknown: NisCas7x3 sequence"
LATLTRHEMVAIDRFTGGGKEGAKFNVDYIECPTLTGAIYLDL

SEQ ID NO: 532 - protein, 43 aa, unidentified
  REGION 1..43: note = source = /note="Description of Unknown: GwCas7x3 sequence"
SIKTRRHEMLAIDRFTGGGKDGAKFNVDYVECPTLAGKLSLDL

SEQ ID NO: 533 - protein, 43 aa, unidentified
  REGION 1..43: note = source = /note="Description of Unknown: HvmCas7x3 sequence"
EDCDNIQEFVAIDRFTGGAKDKAKFNAEYIGSPRFTGTIALDK

SEQ ID NO: 534 - protein, 43 aa, unidentified
  REGION 1..43: note = source = /note="Description of Unknown: DsbCas7x3 sequence"
TRQETVQDFVAIDRFHGGGKETAKFDASFSWRPQYSILMHIPS

SEQ ID NO: 535 - protein, 43 aa, unidentified
  REGION 1..43: note = source = /note="Description of Unknown: GamCas7x3 sequence"
LKPFIIQEFVAIDRFHGGGKDEAKFNAAHYQAPVFKGKVRVSQ

SEQ ID NO: 536 - protein, 43 aa, unidentified
  REGION 1..43: note = source = /note="Description of Unknown: DesCas7x3 sequence"
FRREQEQTFVAIDRFHGGCKEGALYTIRHAESPRFEGHLVIDP

SEQ ID NO: 537 - protein, 43 aa, unidentified
  REGION 1..43: note = source = /note="Description of Unknown: GabCas7x3 sequence"
YRPAKTQQFVAIDRFHGGGKDGALFSIKYFERPVLKGGISLKL

SEQ ID NO: 538 - protein, 43 aa, unidentified
  REGION 1..43: note = source = /note="Description of Unknown: MetCas7x3 sequence"
KSKKTKQDFVAIDRFHGGGKDGAKFDATHFERPEFEGAISFSP

SEQ ID NO: 539 - protein, 43 aa, unidentified
  REGION 1..43: note = source = /note="Description of Unknown: MebCas7x3 sequence"
ANELKTQEFIAIDRFHGGGKDGAKFNAKHSERPYFQGRITLSP

SEQ ID NO: 540 - protein, 47 aa, unidentified
  REGION 1..47: note = source = /note="Description of Unknown: GwCas7-11 sequence"
TPHAMRSDRVALDVFGGAMPEAKFDDRPLAASPGKPLNFKSTIWYRE

SEQ ID NO: 541 - protein, 48 aa, unidentified
  REGION 1..48: note = source = /note="Description of Unknown: HreCas7-11 sequence"
EVEAIKCDHVAIDRFHGGTVHRMKYDDYPLPGSPNRPLRIKGNIWVKR

SEQ ID NO: 542 - protein, 48 aa, unidentified
  REGION 1..48: note = source = /note="Description of Unknown: CmaCas7-11 sequence"
NVSDCCIDHVAIDRFTGGGVEKMKFNDYPLSASPKNCLNLKGSIWITS

SEQ ID NO: 543 - protein, 48 aa, unidentified
  REGION 1..48: note = source = /note="Description of Unknown: HvmCas7-11 sequence"
SVSDKKMDHVAIDRFTGGGVDQMKFDDYPLPGCPAQPLILEGKFWVKD

SEQ ID NO: 544 - protein, 46 aa, unidentified
  REGION 1..46: note = source = /note="Description of Unknown: SmCas7-11 sequence"
TVADKRLDHVAIDRFDQSVVEKYDDRPLVGSPKQPLVFKGCFWVQT

SEQ ID NO: 545 - protein, 46 aa, unidentified
  REGION 1..46: note = source = /note="Description of Unknown: OmCas7-11 sequence"
TWNDKKIDHVSCSRFDASVVNKFDDRSLVGSPDSPLHFEGTFWLHR

SEQ ID NO: 546 - protein, 48 aa, unidentified
  REGION 1..48: note = source = /note="Description of Unknown: DisCas7-11 sequence"
DPEPVTFDHVAIDRFTGGAADKKKFDDSPLPGSPARPLMLKGSFWIRR

SEQ ID NO: 547 - protein, 48 aa, unidentified
  REGION 1..48: note = source = /note="Description of Unknown: DsbaCas7-11 sequence"
EPEPRRFDHVAIDRFTGGAVNQKKFDDRSLVPGKEGFMTLIGCFWMRK

SEQ ID NO: 548 - protein, 48 aa, unidentified
  REGION 1..48: note = source = /note="Description of Unknown: SstCas7-11 sequence"
QPEPMIFDHVAIDRFTGGAVDKKKFDDCSLPGTPGHPLTLKGCFWIRK

SEQ ID NO: 549 - protein, 48 aa, unidentified
  REGION 1..48: note = source = /note="Description of Unknown: SybCas7-11 sequence"
AWTRKHLDHVAIDRFHGGAEENMKFDTYALAASPTNPLRMKGLIWVRS

SEQ ID NO: 550 - protein, 48 aa, unidentified
  REGION 1..48: note = source = /note="Description of Unknown: CsbCas7-11 sequence"
EKLEKHIDHVAIDRFTGGALDKAKFDTYPLAGSPKKPLKLKGRFWIKK

SEQ ID NO: 551 - protein, 48 aa, unidentified
  REGION 1..48: note = source = /note="Description of Unknown: CjcCas7-11 sequence"
SYKKKLIDHVAIDRFHGGAEDKMKFNTLPLAGSFEKPIILKGRFWIKK

SEQ ID NO: 552 - protein, 48 aa, unidentified
  REGION 1..48: note = source = /note="Description of Unknown: CbfCas7-11 sequence"
KDIAKKIDHVAIDRFTGGARDQMKFDTLPLIGSPERPLRLKGLFWMRR

SEQ ID NO: 553 - protein, 48 aa, unidentified
  REGION 1..48: note = source = /note="Description of Unknown: HvsCas7-11 sequence"
GQEEKFFDHVAIDRFLGGAKEKYKFDDKPIIGAPDTPIVLEGKIWVKK

SEQ ID NO: 554 - protein, 48 aa, unidentified
  REGION 1..48: note = source = /note="Description of Unknown: HsmCas7-11 sequence"
DSAPKRLDHVAIDRFTGGAKEQAKFDDSPLIGSPDSPLEFTGIVWVRD

SEQ ID NO: 555 - protein, 48 aa, unidentified
  REGION 1..48: note = source = /note="Description of Unknown: FmCas7-11 sequence"
EIKTYRMDHVAIDRISGGAVDQCKYDDEPLVGTSKHPLVFKGMFWINR

SEQ ID NO: 556 - protein, 45 aa, unidentified
  REGION 1..45: note = source = /note="Description of Unknown: DpbaCas7-11 sequence"
EVREKLFDHVSIDRFTGGAANKLKFDDKPLVGNPLVFQGVFWVHQ

SEQ ID NO: 557 - protein, 48 aa, unidentified
  REGION 1..48: note = source = /note="Description of Unknown: WmCas7x3 sequence"
PVAGFALQNQPAIPATSLRGLLSSLFESISGSNLRVLHPTPYSIRKTT

SEQ ID NO: 558 - protein, 48 aa, unidentified
  REGION 1..48: note = source = /note="Description of Unknown: NisCas7x3 sequence"
EARPFKLNGKHAIPATSLRGMLSSLFESVSNSNFRVLHPEHYSVRKSL

SEQ ID NO: 559 - protein, 48 aa, unidentified
  REGION 1..48: note = source = /note="Description of Unknown: GwCas7x3 sequence"
SLHPFKLNNGLAIPATSLRGMISSLFESVSNSNFRVLDEKTYSMRKTM

SEQ ID NO: 560 - protein, 48 aa, unidentified
  REGION 1..48: note = source = /note="Description of Unknown: HvmCas7x3 sequence"
EILPFTLGGKPAIPATSLRGMLSSIAEAASNSSLRVLEDKTLSYRKSM

SEQ ID NO: 561 - protein, 48 aa, unidentified
  REGION 1..48: note = source = /note="Description of Unknown: DsbCas7x3 sequence"
QVDHYTENGEIAIPATTLRGLLSSLSEAASNSSMRVLDDGMMSYRQPV

SEQ ID NO: 562 - protein, 48 aa, unidentified
  REGION 1..48: note = source = /note="Description of Unknown: GamCas7x3 sequence"
LRHPYQLDGEPAIPSTSLRGLISTMTEAAANCAMRVLDSEIISYRKPM

SEQ ID NO: 563 - protein, 48 aa, unidentified
  REGION 1..48: note = source = /note="Description of Unknown: DesCas7x3 sequence"
RIENYRLGNRIAIPAASLRGMLSSLAEAASNSAMRVLHQGILSYRKKA

SEQ ID NO: 564 - protein, 48 aa, unidentified
  REGION 1..48: note = source = /note="Description of Unknown: GabCas7x3 sequence"
LKEHYQLNNKLAIPATSLRGLISSLAEAASNSALRVLDNGVLSYRKPA

SEQ ID NO: 565 - protein, 48 aa, unidentified
  REGION 1..48: note = source = /note="Description of Unknown: MetCas7x3 sequence"
EINNYRLNGELAIPATSLRGMISSLAEAASNSAMRVLDNGLLSYRKTA

SEQ ID NO: 566 - protein, 48 aa, unidentified
  REGION 1..48: note = source = /note="Description of Unknown: MebCas7x3 sequence"
ELDNYRLNGQLAIPATSLRGMISSLAEAASNSAMRVLDNGLLSYRKDA

SEQ ID NO: 567 - protein, 48 aa, unidentified
  REGION 1..48: note = source = /note="Description of Unknown: GwCas7-11 sequence"
TQRFHQINDEIGLPGASLRGMVLSNYQILTNSCYRNLKATEEITRRMP

SEQ ID NO: 568 - protein, 48 aa, unidentified
  REGION 1..48: note = source = /note="Description of Unknown: HreCas7-11 sequence"
NYRFFRINDELAIPGSELRGMVSSVYEALTNSCFRIMEEGRYLSRRMG

SEQ ID NO: 569 - protein, 48 aa, unidentified
  REGION 1..48: note = source = /note="Description of Unknown: CmaCas7-11 sequence"
NYAFFRINDHIAIPGASIRGMISSVFETLTHSCFRVMDDKKYLTRRVI

SEQ ID NO: 570 - protein, 48 aa, unidentified
  REGION 1..48: note = source = /note="Description of Unknown: HvmCas7-11 sequence"
NYGFFRINGNVAIPGSSIRGMISSVFEALTNSCFRVFDQERYLSRSEK

SEQ ID NO: 571 - protein, 48 aa, unidentified
  REGION 1..48: note = source = /note="Description of Unknown: SmCas7-11 sequence"
NFPFFKINDEIMLPGAPLWAAVSQVYEALTNSCFRVMKQKRFLSWRME

SEQ ID NO: 572 - protein, 48 aa, unidentified
  REGION 1..48: note = source = /note="Description of Unknown: OmCas7-11 sequence"
EYPSFRLNNTPMIPGAGLRAAVSQVYEVLTNSCIRIMDQGQTLSWRMS

SEQ ID NO: 573 - protein, 48 aa, unidentified
  REGION 1..48: note = source = /note="Description of Unknown: DisCas7-11 sequence"
SYAFFRLHKQIMIPGSELRGMVSSVYETVTNSCFRIFDETKRLSWRMD

SEQ ID NO: 574 - protein, 48 aa, unidentified
  REGION 1..48: note = source = /note="Description of Unknown: DsbaCas7-11 sequence"
SYRFFTLNRVPMIPGSEIRGMISSVFEALSNSCFRIFDEKYRLSWRMD

SEQ ID NO: 575 - protein, 48 aa, unidentified
  REGION 1..48: note = source = /note="Description of Unknown: SstCas7-11 sequence"
SYAFFSVNGDIMLPGSEIRGMLSSVYEALTNSCFRVFDEGYRLSWRME

SEQ ID NO: 576 - protein, 48 aa, unidentified
  REGION 1..48: note = source = /note="Description of Unknown: SybCas7-11 sequence"
MCKFFSVAGKPMIPGSEIRGMISSVYEALTNSCFRVFDEEKYLTRRVQ

SEQ ID NO: 577 - protein, 48 aa, unidentified
  REGION 1..48: note = source = /note="Description of Unknown: CsbCas7-11 sequence"
NYKFFNINGELMIPGSELRGMLRTHFEALTKSCFAIFGEDSTLSWRMN

SEQ ID NO: 578 - protein, 48 aa, unidentified
  REGION 1..48: note = source = /note="Description of Unknown: CjcCas7-11 sequence"
NYQFFHINDEIMVPGSEIRGMISSVYEAITNSCFRVYDETKYITRRLS

SEQ ID NO: 579 - protein, 48 aa, unidentified
  REGION 1..48: note = source = /note="Description of Unknown: CbfCas7-11 sequence"
TYDFFQMNNAIMIPGSEIRGMISAVYEAMTNSCFRIFHEKQYLTRRIS

SEQ ID NO: 580 - protein, 48 aa, unidentified
  REGION 1..48: note = source = /note="Description of Unknown: HvsCas7-11 sequence"
YYHFLKIDNKPIIPGAEIRGAVSSIYEALTNSCFRVFGEKKVLSWRME

SEQ ID NO: 581 - protein, 48 aa, unidentified
  REGION 1..48: note = source = /note="Description of Unknown: HsmCas7-11 sequence"
VYKFFRLGGHLCIPAAEIRGMISSVYEALTNSCFRVFDEKRLISWRMT

SEQ ID NO: 582 - protein, 48 aa, unidentified
  REGION 1..48: note = source = /note="Description of Unknown: FmCas7-11 sequence"
SFRFFRIDDEVLIPGSEIRGMVSTVFEALTGSCFRVINQKAHLSWRIN

SEQ ID NO: 583 - protein, 48 aa, unidentified
  REGION 1..48: note = source = /note="Description of Unknown: DpbaCas7-11 sequence"
RFKFMRMGSQAAIPGSAIRSMTSSVFEALTNSCFRVLDQKSHLSWRME

SEQ ID NO: 584 - protein, 21 aa, unidentified
  REGION 1..21: note = source = /note="Description of Unknown: WmCas7x3 sequence"
EALSAIGRIVERNGELKLYPL

SEQ ID NO: 585 - protein, 23 aa, unidentified
  REGION 1..23: note = source = /note="Description of Unknown: NisCas7x3 sequence"
DYVALSAMGRIVDDQGELKLQPL

SEQ ID NO: 586 - protein, 21 aa, unidentified
  REGION 1..21: note = source = /note="Description of Unknown: GwCas7x3 sequence"
QSLSAMGRIVRHDQKLYLLPL

SEQ ID NO: 587 - protein, 31 aa, unidentified
  REGION 1..31: note = source = /note="Description of Unknown: HvmCas7x3 sequence"
ANRNEDKPLSALGMIKKIETGDKVEYRLLPL

SEQ ID NO: 588 - protein, 22 aa, unidentified
  REGION 1..22: note = source = /note="Description of Unknown: DsbCas7x3 sequence"
SGSLSAIGMVVIRDGKKFIYPL

SEQ ID NO: 589 - protein, 23 aa, unidentified
  REGION 1..23: note = source = /note="Description of Unknown: GamCas7x3 sequence"
PSHILSALGMVTKRGEDFWLIPL

SEQ ID NO: 590 - protein, 21 aa, unidentified
  REGION 1..21: note = source = /note="Description of Unknown: DesCas7x3 sequence"
NALREIGMIVLRDGKRFILPL

SEQ ID NO: 591 - protein, 21 aa, unidentified
  REGION 1..21: note = source = /note="Description of Unknown: GabCas7x3 sequence"
RALRKIGILFKREEQWRLVQM

SEQ ID NO: 592 - protein, 21 aa, unidentified
  REGION 1..21: note = source = /note="Description of Unknown: MetCas7x3 sequence"
DALRKVGMVIYVDNKSFIIKL

SEQ ID NO: 593 - protein, 22 aa, unidentified
  REGION 1..22: note = source = /note="Description of Unknown: MebCas7x3 sequence"
LALSKIGITFINRQGQWQLIPM

SEQ ID NO: 594 - protein, 27 aa, unidentified
  REGION 1..27: note = source = /note="Description of Unknown: GwCas7-11 sequence"
DEAKYRKAGRVTVSGDGAQKKYSIQEM

SEQ ID NO: 595 - protein, 20 aa, unidentified
  REGION 1..20: note = source = /note="Description of Unknown: HreCas7-11 sequence"
DEFKDFHPGIVVDGAKIREM

SEQ ID NO: 596 - protein, 39 aa, unidentified
  REGION 1..39: note = source = /note="Description of Unknown: CmaCas7-11 sequence"
ESETTQKRKSGRYQVEESDPDLFPGRVQKKGNKYKIEKM

SEQ ID NO: 597 - protein, 25 aa, unidentified
  REGION 1..25: note = source = /note="Description of Unknown: HvmCas7-11 sequence"
DPTELTKYYPGKVKRDGNKFFILKM

SEQ ID NO: 598 - protein, 21 aa, unidentified
  REGION 1..21: note = source = /note="Description of Unknown: SmCas7-11 sequence"
EDYKDFYPGRVLDGGKQIKKM

SEQ ID NO: 599 - protein, 21 aa, unidentified
  REGION 1..21: note = source = /note="Description of Unknown: OmCas7-11 sequence"
SEHKDYQPGKITDNGRKIQPM

SEQ ID NO: 600 - protein, 24 aa, unidentified
  REGION 1..24: note = source = /note="Description of Unknown: DisCas7-11 sequence"
DHQNVLQDFLPGRVTADGKHIQKF

SEQ ID NO: 601 - protein, 23 aa, unidentified
  REGION 1..23: note = source = /note="Description of Unknown: DsbaCas7-11 sequence"
DVKELEQFKPGRVADDGKRIEEM

SEQ ID NO: 602 - protein, 23 aa, unidentified
  REGION 1..23: note = source = /note="Description of Unknown: SstCas7-11 sequence"
DRNVLMQFKPGRVTDNGLRIEEM

SEQ ID NO: 603 - protein, 27 aa, unidentified
  REGION 1..27: note = source = /note="Description of Unknown: SybCas7-11 sequence"
KKGAKSSELVPGIIVWGQNGGLAVQQV

SEQ ID NO: 604 - protein, 45 aa, unidentified
  REGION 1..45: note = source = /note="Description of Unknown: CsbCas7-11 sequence"
DEKDYKIDSNSIRKMESQRNPKYRIPDELQKELRNSGNGLFNRLY

SEQ ID NO: 605 - protein, 37 aa, unidentified
  REGION 1..37: note = source = /note="Description of Unknown: CjcCas7-11 sequence"
EKKDESNDKNKSQDDASQKIRKGLVKKTDEGFSIIEV

SEQ ID NO: 606 - protein, 25 aa, unidentified
  REGION 1..25: note = source = /note="Description of Unknown: CbfCas7-11 sequence"
EDKELREFIPGIVRIINGDVYIEKA

SEQ ID NO: 607 - protein, 23 aa, unidentified
  REGION 1..23: note = source = /note="Description of Unknown: HvsCas7-11 sequence"
KDAKEFMPGRVSKKKGKLYMVKM

SEQ ID NO: 608 - protein, 36 aa, unidentified
  REGION 1..36: note = source = /note="Description of Unknown: HsmCas7-11 sequence"
EEAKRPDPKKSEEQNRMRFRPGRIIKKDKKFYAQEM

SEQ ID NO: 609 - protein, 23 aa, unidentified
  REGION 1..23: note = source = /note="Description of Unknown: FmCas7-11 sequence"
DMAKHYRPGRIIQNNEKMFIQPY

SEQ ID NO: 610 - protein, 23 aa, unidentified
  REGION 1..23: note = source = /note="Description of Unknown: DpbaCas7-11 sequence"
DDAGDYKPGRFEKKDDKAVIRKF

SEQ ID NO: 611 - protein, 1291 aa, unidentified; identical to SEQ ID NO: 23
  REGION 1..1291: note = source = /note="Description of Unknown: WmCas7x3 sequence"

SEQ ID NO: 612 - protein, 1437 aa, Nitrosomonas sp.
MTDTVKSGRWIITGQFQLVTPMHIGTGLDEEMDKQSGESVDKKQNNSWIQAIALDLNKKPYIPGASIKGALKALARRYYCASNLNIFGDTIDTKDGDNKRKSVTVAGQAEFLNAWYAADQEDKPFDTITRVAIDRVTGTAEDRKLFNTRRVNPGVCFNYKIIIQNACETEIQYLLDLLRKAAKDPSFSLGAGANQSQGKVRCLSSCVRYFGKQEMHDWFRAIQNGKQEHWQIFAKPSNIKYADLERPDIIANSLNLPLTLDFHTPFLVKASKKKDEAKNEADAKPRTNHQGQVILPASSLRGRLRAQAEKILRTMGQDIPQGHAAPAYDGIAHRDLISLLFGTAGWKGIVCASDLIHSIPEYALQFNGVRETISDLSDTVKSCIIVDLVKTSTAAEKTEEQLHIRIVDSAGSLIVHKSENSSWANDTFRDASVKDNFKARLKEIADPQDLSDALRADIKKRAFQLATLTRHEMVAIDRFTGGGKEGAKFNVDYIECPTLTGAIYLDLHRLKQAQLKNDEDALKPALGLISLLLRDLAEGDIAFGFGANKGYGQCREHAVLDNWEERLKKIGAGLTIDGALQALRDTVALEPPAEFPPEIEKTTDDNQPEAPDFNLKPASNGFHNPYHFIPLNNPKIGDWPEAKAETLKANREGHDQYHTGKFSGRIVCSLTTQTPLFIGAETKPSTSDREPSEARPFKLNGKHAIPATSLRGMLSSLFESVSNSNFRVLHPEHYSVRKSLDDYVALSAMGRIVDDQGELKLQPLTLPTLFGNRNNVPAKWEKIFGTPSEDDFLRIYFDDIPSKFSSNKRYFYNCKATELKDFIKSDKYFIGKRTPTVFPKSSTEKSHLESLEFIDVEKFKKAVENLEITPGNNPYIHGWVRNLKDEFREDIPDNVKHHVFLPDTTKRVSPLEIPPHVKKRFHELADLALAGLHLKQGETIASPYKILPYTPIGRNKLENHIHRVPNDLTCYMTRLKKGDLVFFDVDNDGQITEISFSSIWRAGIGTKNKLQTTADLLSQRDPNLVQLGMGVRTKNTDRFKLSPAERLFGVVEHRDDDNTTVENVNQPNDKAQAFAFAGKVRIGFGLPDKKTTVNGVSPVTLKELSSPKPPSPAFYLKRKNNDDFVSKKVAAECSETMTLRGRKCYLHAWREQNGNVMKLDAIGVNSGGSTCKPPWKTHKPAANDQKEFEEDKNKFITSRQVKIAPISENTPFYFEIDFNNLDATELAQLCATLQPAPKFEHRLGMGKPLGLGSVKIEPVGLFLINRHQRYTTDSTNCDRYHYAWLKGEHAAWDWPEYFRQNVVTADCTQTFNDTFDKLVQNGLAGTDADIKHALQLLGDPQYIGVPVHYPIAGNSTLENKHFEWFGNNDKASVLRQKAQANSKNHHYQPKQQATPEEPQYLHTITKDSKQISLLKKNKIEDIENRDQQKHRYSNHRR

SEQ ID NO: 613 - protein, 1237 aa, unidentified; identical to SEQ ID NO: 22
  REGION 1..1237: note = source = /note="Description of Unknown: GwCas7x3 sequence"

SEQ ID NO: 614 - protein, 1276 aa, unidentified; identical to SEQ ID NO: 30
  REGION 1..1276: note = source = /note="Description of Unknown: Gammaproteobacteria bacterium sequence"

SEQ ID NO: 615 - protein, 1221 aa, Methylobacter tundripaludum
MNLPTWKLNNEKRWHISIVLTTATPLHIGSGEFCEHDDVKNNDGEPVKINACIKGSKGRPIIPGSTIKGKLYEWLKTRNTEENLLEKLFGKGHNSVSQDQGRGGKAEFHDAEIIEPLTGSQPWPYWREEHQAFIAASTAIDRHKQVALQQSLHYMETVPAGIRFKFTFTGVMRDEEAALLIAALDSFDKNQNQPCFGVDRANAYGRMELHGHLHVKVMGATEISSWLNSFSENDKKMAMESARNLEQQEINTLIEQGNALFKASCDEVKLGLTLKFKGPFLVNDPYAVKILSSNENAKTDHYPLLDKNRNPYLPVSSFRGVLRSQAERIIRTLGGKCCSTDDPCKPIFDKGDLSKLCLACQIFGASGWKTVINIHDFKAINKSKKTKQDFVAIDRFHGGGKDGAKFDATHFERPEFEGAISFSPRMANNDLDWGKGLLALVLRDMQEGDMTFGYGANKGYGGLESASITGIEQITSDIQAFRDKCVASPQTWLCDEAVKPANQQDKIPPAGIQVANSGFHNPYQFIPSKEPDTGHWLPVLGLNADSHHSHAFYRDQTDNGEKLYHGRLICCLNTETPIFIGADKKKDTEPAEINNYRLNGELAIPATSLRGMISSLAEAASNSAMRVLDNGLLSYRKTADDALRKVGMVIYVDNKSFIIKLNDAIKLKQTYTPGNMKDFIEKSNSWSPEHNTVYYLDNNQIPQESYMNGMKPGILRILGKEGREQELENKLHELFIPVPLEYVDTENNKFDYQAYKKAFLYRAIEIPEPVLKRYSELADQRTMSQKSNKELKKDDTCQSVGWLPFHLKGTKRQLDDKHNVGKLQIDEYDLIYYEASGKEVTEVAFSSIWRGRVETNSSQANKVYSFIPGELLPFNESRKKVSPAELLFGFTQINKDGSKADDKAQAFAGKVRISAGTISEYPENEANLLEQEVTLKALSTPKLPSPALYFRTINGNGSAYISKQELEPSKHLAKGRKYYLHALRTGDNKVQKLGSQGETANGGDSKLPWVTHNPDERPQLKVKIKPIKAEFIFSLDFNNLTEWELGLLCYALRPTDSFRHRIGMGKPLGLGSVKIDIMALQTINRQQRYAQDGLEENRFNRHNWVNPPHQPRLEKAGYSISLSSTPLNPEILRATFTKTMNADIYRTLELLGNPQNVKRPVHYPQVENHNIEQENYKWFVANDQGSGKGRNKIDPAEKALKILTENSDCLPTLSRLDWRDE

SEQ ID NO: 616 - protein, 1225 aa, Desulfobulbus sp.; identical to SEQ ID NO: 26

SEQ ID NO: 617 - protein, 1720 aa, unidentified; identical to SEQ ID NO: 19
  REGION 1..1720: note = source = /note="Description of Unknown: HreCas7.11 sequence"

SEQ ID NO: 618 - protein, 1657 aa, unidentified; identical to SEQ ID NO: 4
  REGION 1..1657: note = source = /note="Description of Unknown: CmaCas7.11 sequence"

SEQ ID NO: 619 - protein, 1723 aa, unidentified; identical to SEQ ID NO: 17
  REGION 1..1723: note = source = /note="Description of Unknown: HvmCas7.11 sequence"

SEQ ID NO: 620 - protein, 1575 aa, unidentified; identical to SEQ ID NO: 21
  REGION 1..1575: note = source = /note="Description of Unknown: SmCas7x3 sequence"

SEQ ID NO: 621 - protein, 1801 aa, unidentified; identical to SEQ ID NO: 24
  REGION 1..1801: note = source = /note="Description of Unknown: oral_meta sequence"

SEQ ID NO: 622 - protein, 1601 aa, unidentified; identical to SEQ ID NO: 5
  REGION 1..1601: note = source = /note="Description of Unknown: DisCas7.11 sequence"

SEQ ID NO: 623 - protein, 1659 aa, unidentified; identical to SEQ ID NO: 3
  REGION 1..1659: note = source = /note="Description of Unknown: DsbaCas7.11 sequence"

SEQ ID NO: 624 - protein, 1652 aa, unidentified; identical to SEQ ID NO: 16
  REGION 1..1652: note = source = /note="Description of Unknown: SstCas7.11 sequence"

SEQ ID NO: 625 - protein, 1717 aa, unidentified; identical to SEQ ID NO: 2
  REGION 1..1717: note = source = /note="Description of Unknown: CsbCas7.11 sequence"

SEQ ID NO: 626 - protein, 1812 aa, unidentified; identical to SEQ ID NO: 1
  REGION 1..1812: note = source = /note="Description of Unknown: CjcCas7.11 sequence"

SEQ ID NO: 627 - protein, 1573 aa, unidentified; identical to SEQ ID NO: 6
  REGION 1..1573: note = source = /note="Description of Unknown: CbfCas7.11 sequence"

SEQ ID NO: 628 - protein, 1528 aa, unidentified; identical to SEQ ID NO: 15
  REGION 1..1528: note = source = /note="Description of Unknown: HvsCas7.11 sequence"

SEQ ID NO: 629 - protein, 1548 aa, unidentified; identical to SEQ ID NO: 18
  REGION 1..1548: note = source = /note="Description of Unknown: HsmCas7.11 sequence"

SEQ ID NO: 630 - protein, 1610 aa, unidentified; identical to SEQ ID NO: 20
  REGION 1..1610: note = source = /note="Description of Unknown: FmCas7.11 sequence"

SEQ ID NO: 631 - protein, 1526 aa, unidentified; identical to SEQ ID NO: 7
  REGION 1..1526: note = source = /note="Description of Unknown: DpbaCas7.11 sequence"

SEQ ID NO: 632 - protein, 1329 aa, unidentified
  REGION 1..1329: note = source = /note="Description of Unknown: Syntrophorhabdaceae bacterium sequence"
MFPKGRQMRRQRLLGDAEYYGGTGREQPASIVISTDSDPDHKVYEWIITGQLKAETGFFFGTKAGAGGHTDLSILLGKDGHYRVPRSVFRGALRRDLRVAFGAGCRVEVGRERPCECPVCKVMRQITVMDTISSYREAPEIRQRIRLNPYTGTVDKGALFDMEVGPEGIEFPFVLRFRGSKSFPSELAAVIGSWTKGTAWLGGAAATGKGRFSLLGLSIHKWNLSTAEGRKSYLAAYGLRDAADKTVKRLSIDKGGKGDVGLPAGLERDALPSSVREPLWKKLVCTVDFSSPLLLADPIAALLGVEGDERIGFDNIAYEKRRYNGETNTTESIPAVKGETFRGIVRTALGKRHGNLTRDHEDCRCRLCAVFGKEQEAGKIRFEDLMPVGAWTRKHLDHVAIDRFHGGAEENMKFDTYALAASPTNPLRMKGLIWVRSDLFETGHDGPTPPYVKDIIDALADVKRGLYPVGGKTGSGYGWIKDVTIDGLPQGLSLPPAEERVDGVNEVPPYNYSAPPDLPSAAEGEYFFPHVFIKPYDKVDRVSRLTGHDRFRQGRITGRITCTLKTLTPLIIPDSEGIQTDATGHKMCKFFSVAGKPMIPGSEIRGMISSVYEALTNSCFRVFDEEKYLTRRVQPKKGAKSSELVPGIIVWGQNGGLAVQQVKNAYRVPLYDDPAVTSAIPTEAQKNKERWESVPSVNLQGALDWNLTTANIARDNRTFLNSRPEEKDAILSGTKPISFELEGTNPNDMLVRLVPDGVDGAHSGYLKFTGLNMVLKANKKTSRKLAPSEEDVRTLAILHNDFDSRRDWRRPPNSQRYFPRSVLRFSLERSTYTIPKRCERVFEGTCGEPYSVPSDVERQYNSIIDDISKNYGRISETYLTKTANRKLTVGDLVYFIADLDKNMATHILPVFISRISDEKPLGELLPFSGKLIPCEGEPPTILKKMAPSLLTEAWRTLISTHLEGFCPACRLFGTTSYKGRIRFGFAEHTGTPKWLREELDWARPFLTLPIQERPRPTWSVPDDKSEVPGRKFYLHHHGGNRIVESNLRNRPEVNQTKNNSSVEPISAGNTFTFDVCFENLEAWELGLLLYCLELSPKLAHKLGRAKAFGFGSVKIHVERIEERTTDGAYQDVTAVKKNGWITTGHDKLREWFHRDDWEDVDHIRNLRTVLRFPDADQEHDVRYPELKANNGVSGYVELRDKMTASERQESLRTPWYRWFPQNGTGGSGRHEQAATSQEQDTAKDESVLSATQRRQAVIDVSDPDERLSGTVESFDRQKGDGYIGCGVRQFYVRLEDIRSRTALCEGQVVTFRARKEWEGHEAYDVEIDQ

SEQ ID NO: 633 - RNA, 45 nt, synthetic construct
  misc_feature 1..45: note = source = /note="Description of Artificial Sequence: Synthetic oligonucleotide"
  source 1..45: mol_type = other RNA
  variation 15..45: note = a, c, u, g, unknown or other
tgatgtcacggaacnnnnnnnnnnnnnnnnnnnnnnnnnnnnnnn

SEQ ID NO: 634 - RNA, 35 nt, synthetic construct
  misc_feature 1..35: note = source = /note="Description of Artificial Sequence: Synthetic oligonucleotide"
  source 1..35: mol_type = other RNA
ggttggaaagccggttttctttgatgtcacggaac

SEQ ID NO: 635 - DNA, 54 bp, synthetic construct
  misc_feature 1..54: note = source = /note="Description of Artificial Sequence: Synthetic oligonucleotide"
  source 1..54: mol_type = other DNA
  variation 10..15: note = a, c, t, g, unknown or other
  variation 47..52: note = a, c, t, g, unknown or other
ctagaaatannnnnntggattacttggtagaacaccaatctactcgnnnnnnac

SEQ ID NO: 636 - DNA, 31 bp, synthetic construct
  misc_feature 1..31: note = source = /note="Description of Artificial Sequence: Synthetic oligonucleotide"
  source 1..31: mol_type = other DNA
cgagtagattggtgttctaccaagtaatcca

SEQ ID NO: 637 - RNA, 36 nt, synthetic construct
  misc_feature 1..36: note = source = /note="Description of Artificial Sequence: Synthetic oligonucleotide"
  source 1..36: mol_type = other RNA
gtttggaagccgggtcgaagtcaggcccgttaagac

SEQ ID NO: 638 - RNA, 36 nt, synthetic construct
  misc_feature 1..36: note = source = /note="Description of Artificial Sequence: Synthetic oligonucleotide"
  source 1..36: mol_type = other RNA
cttgaagactaaaggaaggaattaatgtcacggtac

SEQ ID NO: 639 - RNA, 45 nt, synthetic construct
  misc_feature 1..45: note = source = /note="Description of Artificial Sequence: Synthetic oligonucleotide"
  source 1..45: mol_type = other RNA
attgaatgcagagtagaggccgcaggatagtttagaacattctat

SEQ ID NO: 640 - DNA, 46 bp, synthetic construct
  misc_feature 1..46: note = source = /note="Description of Artificial Sequence: Synthetic oligonucleotide"
  misc_feature 1..46: note = source = /note="Description of Combined DNA/RNA Molecule: Synthetic oligonucleotide"
  source 1..46: mol_type = other DNA
gttgatgtcacggaactctaaaccatcctgcggcctctactctgca

SEQ ID NO: 641 - DNA, 101 bp, synthetic construct
  misc_feature 1..101: note = source = /note="Description of Artificial Sequence: Synthetic polynucleotide"
  source 1..101: mol_type = other DNA
ggttggaaagccggttttctttgatgtcacggaacctttgttgtcttcgacatgggtaatcctcatggttggaaagccggttttctttgatgtcacggaac

SEQ ID NO: 642 - DNA, 65 bp, synthetic construct
  misc_feature 1..65: note = source = /note="Description of Artificial Sequence: Synthetic oligonucleotide"
  source 1..65: mol_type = other DNA
gttggaaagccggttttctttgatgtcacggaaccgagtagattgctgttctaccaagtaatcca

SEQ ID NO: 643 - DNA, 46 bp, synthetic construct
  misc_feature 1..46: note = source = /note="Description of Artificial Sequence: Synthetic oligonucleotide"
  source 1..46: mol_type = other DNA
ttgatgtcacggaacctttgttgtcttcgacatgggtaatcctcat

SEQ ID NO: 644 - RNA, 37 nt, synthetic construct
  misc_feature 1..37: note = source = /note="Description of Artificial Sequence: Synthetic oligonucleotide"
  source 1..37: mol_type = other RNA
  variation 16..37: note = a, c, u, g, unknown or other
ttaatgtcacggtacnnnnnnnnnnnnnnnnnnnnnn

SEQ ID NO: 645 - DNA, 50 bp, synthetic construct
  misc_feature 1..50: note = source = /note="Description of Artificial Sequence: Synthetic oligonucleotide"
  source 1..50: mol_type = other DNA
caggttcctggaactggaatctctttccatagaatgttctaaaccatcct

SEQ ID NO: 646 - DNA, 50 bp, synthetic construct
  misc_feature 1..50: note = source = /note="Description of Artificial Sequence: Synthetic oligonucleotide"
  source 1..50: mol_type = other DNA
accaggttcctggaactggaatctctttccatagaatgttctaaaccatc

SEQ ID NO: 647 - DNA, 50 bp, synthetic construct
  misc_feature 1..50: note = source = /note="Description of Artificial Sequence: Synthetic oligonucleotide"
  source 1..50: mol_type = other DNA
gtaccaggttcctggaactggaatctctttccatagaatgttctaaacca